(19) United States
(12) Patent Application Publication
(10) Pub. No.: US 2005/0196784 A1
(43) Pub. Date: Sep. 8, 2005

(54) Human Type II diabetes gene - Kv channel-interacting protein (KChIP1) located on chromosome 5

(75) Inventors: Inga Reynisdottir, Reykjavik (IS); Jeffrey R. Gulcher, Lake Barrington, IL (US); Struan F. Grant, Reykjavik (IS); Gudmar Thorleifsson, Reykjavik (IS)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: deCODE genetics ehf., Reykjavik (IS)

(21) Appl. No.: 11/029,984

(22) Filed: Jan. 5, 2005

Related U.S. Application Data
(63) Continuation-in-part of application No. 10/976,368, filed on Oct. 28, 2004.
Application No. 10/976,368 is a continuation-in-part of application No. 10/820,226, filed on Apr. 7, 2004.
Application No. 10/820,226 is a continuation-in-part of application No. PCT/US03/34681, filed on Oct. 31, 2003.
(60) Provisional application No. 60/477,111, filed on Jun. 9, 2003.
(60) Provisional application No. 60/449,945, filed on Feb. 25, 2003.
(60) Provisional application No. 60/423,545, filed on Nov. 1, 2002.

(51) Int. Cl. C12Q 1/68

(52) U.S. Cl. 435/6

(57) ABSTRACT

Association of Type II diabetes and a locus on chromosome 5 is disclosed. In particular, the gene KChIP1 within this locus is shown by linkage analysis to be a susceptibility gene for Type II diabetes. Pathway targeting for drug delivery and diagnosis applications in identifying those who have Type II diabetes or are at risk of developing Type II diabetes, in particular those that are non-obese are described.

### RELATED APPLICATIONS

This application is a continuation-in-part of U.S. application Ser. No. 10/976,368, filed Oct. 28, 2004, which is a continuation-in-part of U.S. application Ser. No. 10/820,226, filed Apr. 7, 2004, which is a continuation-in-part of International Application No. PCT/US03/34681, which designated the United States and was filed on Oct. 31, 2003, published in English, which claims priority to U.S. Provisional Application No. 60/477,111 filed Jun. 9, 2003, and to U.S. Provisional Application No. 60/449,945, filed on Feb. 25, 2003, and also to U.S. Provisional Application No. 60/423,545, filed on Nov. 1, 2002, the entire contents of all applications are incorporated herein by reference.

### BACKGROUND OF THE INVENTION

Diabetes mellitus, a metabolic disease wherein carbohydrate utilization is reduced and lipid and protein utilization is enhanced, is caused by an absolute or relative deficiency of insulin. In the more severe cases, diabetes is characterized by chronic hyperglycemia, glycosuria, water and electrolyte loss, ketoacidosis and coma. Long term complications include development of neuropathy, retinopathy, nephropathy, generalized degenerative changes in large and small blood vessels and increased susceptibility to infection. The most common form of diabetes is Type II, non-insulin-dependent diabetes that is characterized by hyperglycemia due to impaired insulin secretion and insulin resistance in target tissues. Both genetic and environmental factors contribute to the disease. For example, obesity plays a major role in the development of the disease. Type II diabetes is often a mild form of diabetes mellitus of gradual onset.

The health implications of Type II diabetes are enormous. In 1995, there were 135 million adults with diabetes worldwide. It is estimated that close to 300 million will have diabetes in the year 2025. (King H., et al., Diabetes Care, 21(9): 1414-1431 (1998)). The prevalence of Type II diabetes in the adult population in Iceland is 2.5% (Vilbergsson, S., et al., Diabet. Med., 14(6): 491-498 (1997)), which comprises approximately 5,000 people over the age of 34 who have the disease. The high prevalence of the disease and increasing population affected shows an unmet medical need to define the genetic factors involved in Type II diabetes to more precisely define the associated risk factors. Also needed are therapeutic agents for prevention of Type II diabetes.

### SUMMARY OF THE INVENTION

As described herein, a locus on chromosome 5q35 has been demonstrated to play a major role in Type II diabetes. The locus, referred to as the Type II diabetes locus, comprises a nucleic acid that encodes, KChIP1.

The present invention relates to genes located within the Type II diabetes-related locus, particularly nucleic acids comprising the KChIP1 gene, and the amino acids encoded by these nucleic acids. The invention further relates to pathway targeting for drug delivery and diagnosis of individuals with Type II diabetes and those at risk of developing Type II diabetes. Also described are haplotypes and SNPs that can be used to identify individuals with Type II diabetes or at risk of developing Type II diabetes, particularly in those that are non-obese. As a consequence, intervention, for example, dietary changes, exercise and/or medication, can be prescribed to these individuals before symptoms of the disease present. Identification of genes in the Type II diabetes locus can pave the way for a better understanding of the disease process, which in turn can lead to improved diagnostics and therapeutics.

The present invention pertains to methods of diagnosing a susceptibility to Type II diabetes in an individual, comprising detecting a polymorphism in a KChIP1 nucleic acid, wherein the presence of the polymorphism in the nucleic acid is indicative of a susceptibility to Type II diabetes. The invention additionally pertains to methods of diagnosing Type II diabetes in an individual, comprising detecting a polymorphism in a KChIP1 nucleic acid, wherein the presence of the polymorphism in the nucleic acid is indicative of Type II diabetes. In one embodiment, in diagnosing Type II diabetes or susceptibility to Type II diabetes by detecting the presence of a polymorphism in a KChIP1 nucleic acid, the presence of the polymorphism in the KChIP1 nucleic acid can be indicated, for example, by the presence of one or more of the polymorphisms indicated in Table 10.

In other embodiments, the invention relates to methods of diagnosing a susceptibility to Type II diabetes in an individual, comprising detecting an alteration in the expression or composition of a polypeptide encoded by a KChIP1 nucleic acid in a test sample, in comparison with the expression or composition of a polypeptide encoded by a KChIP1 nucleic acid in a control sample, wherein the presence of an alteration in expression or composition of the polypeptide in the test sample is indicative of a susceptibility to Type II diabetes. The invention additionally relates to a method of diagnosing Type II diabetes in an individual, comprising detecting an alteration in the expression or composition of a polypeptide encoded by a KChIP1 nucleic acid in a test sample, in comparison with the expression or composition of a polypeptide encoded by KChIP1 nucleic acid in a control sample, wherein the presence of an alteration in expression or composition of the polypeptide in the test sample is indicative of Type II diabetes.

The invention also relates to an isolated nucleic acid molecule comprising a KChIP1 nucleic acid (e.g., SEQ ID NO: 1 or the complement of SEQ ID NO:1). In certain embodiments, the KChIP1 nucleic acid comprises one or more nucleotide sequence(s) selected from the group of nucleic acid sequences as shown in Table 10, (e.g., SEQ ID NOs: 114-258), and the complements of the group of nucleic acid sequences as shown in Table 10. For example, in certain embodiments, the nucleotide sequence contains one or more polymorphism(s), such as those shown in Table 10. In another embodiment, the invention relates to an isolated nucleic acid molecule which hybridizes under high stringency conditions to a nucleotide sequence selected from the group of SEQ ID NO: 1 and the complement of SEQ ID NO: 1. In certain embodiments, the isolated nucleic acid molecule hybridizes under high stringency conditions to a nucleotide sequence comprising one or more nucleotide sequence(s) selected from the group of nucleic acid sequences as shown in Table 10 (e.g., SEQ ID NOs: 114-258) and the complements of the group of nucleic acid sequences as shown in Table 10. For example, in certain embodiments, the nucleotide sequence contains one or more polymorphism(s), such as those shown in Table 10.

Also contemplated by the invention is a method of assaying for the presence of a first nucleic acid molecule in a sample, comprising contacting said sample with a second nucleic acid molecule, where the second nucleic acid molecule comprises at least one (or more) nucleic acid sequence(s) selected from the group of SEQ ID NOs: 1 and 114-258, inclusive, wherein the nucleic acid sequence hybridizes to the first nucleic acid under high stringency conditions. In certain embodiments, the second nucleic acid molecule contains one or more polymorphism(s), such as those shown in Table 10.

The invention also relates to a vector comprising an isolated nucleic acid molecule of the invention (e.g., SEQ ID NOs: 1 and 114-258; optionally including one or more of the polymorphisms shown in Table 10) operably linked to a regulatory sequence, as well as to a recombinant host cell comprising the vector. The invention also provides a method for producing a polypeptide encoded by an isolated nucleic acid molecule having a polymorphism, comprising culturing the recombinant host cell under conditions suitable for expression of the nucleic acid molecule.

Also contemplated by the invention is a method of assaying for the presence of a polypeptide encoded by an isolated nucleic acid molecule of the invention in a sample, the method comprising contacting the sample with an antibody that specifically binds to the encoded polypeptide.

The invention further pertains to a method of identifying an agent that alters expression of a KChIP1 nucleic acid, comprising: contacting a solution containing a nucleic acid comprising the promoter region of the KChIP1 gene operably linked to a reporter gene, with an agent to be tested; assessing the level of expression of the reporter gene in the presence of the agent; and comparing the level of expression of the reporter gene in the presence of the agent with a level of expression of the reporter gene in the absence of the agent; wherein if the level of expression of the reporter gene in the presence of the agent differs, by an amount that is statistically significant, from the level of expression in the absence of the agent, then the agent is an agent that alters expression of the KChIP1 gene or nucleic acid. An agent identified by this method is also contemplated.

The invention additionally comprises a method of identifying an agent that alters expression of a KChIP1 nucleic acid, comprising contacting a solution containing a nucleic acid of the invention or a derivative or fragment thereof, with an agent to be tested; comparing expression of the nucleic acid, derivative or fragment in the presence of the agent with expression of the nucleic acid, derivative or fragment in the absence of the agent; wherein if expression of the nucleic acid, derivative or fragment in the presence of the agent differs, by an amount that is statistically significant, from the expression in the absence of the agent, then the agent is an agent that alters expression of the KChIP1 nucleic acid. In certain embodiments, the expression of the nucleic acid, derivative or fragment in the presence of the agent comprises expression of one or more splicing variants(s) that differ in kind or in quantity from the expression of one or more splicing variant(s) the absence of the agent. Agents identified by this method are also contemplated.

Representative agents that alter expression of a KChIP1 nucleic acid contemplated by the invention include, for example, antisense nucleic acids to a KChIP1 gene or nucleic acid; a KChIP1 gene or nucleic acid; a KChIP1 polypeptide; a KChIP1 gene or nucleic acid receptor, or other receptor; a KChIP1 binding agent; a peptidomimetic; a fusion protein; a prodrug thereof; an antibody; and a ribozyme. A method of altering expression of a KChIP1 nucleic acid, comprising contacting a cell containing a nucleic acid with such an agent is also contemplated.

The invention further pertains to a method of identifying a polypeptide which interacts with a KChIP1 polypeptide (e.g., a KChIP1 polypeptide encoded by a nucleic acid of the invention, such as a nucleic acid comprising one or more polymorphism(s) indicated in Table 10), comprising employing a yeast two-hybrid system using a first vector which comprises a nucleic acid encoding a DNA binding domain and a KChIP1 polypeptide, splicing variant, or a fragment or derivative thereof, and a second vector which comprises a nucleic acid encoding a transcription activation domain and a nucleic acid encoding a test polypeptide. If transcriptional activation occurs in the yeast two-hybrid system, the test polypeptide is a polypeptide, which interacts with a KChIP1 polypeptide.

In certain methods of the invention, a Type II diabetes therapeutic agent is used. The Type II diabetes therapeutic agent can be an agent that alters (e.g., enhances or inhibits) KChIP1 polypeptide activity and/or KChIP1 nucleic acid expression, as described herein (e.g., a nucleic acid agonist or antagonist).

Type II diabetes therapeutic agents can alter polypeptide activity or nucleic acid expression of a KChIP1 nucleic acid by a variety of means, such as, for example, by providing additional polypeptide or upregulating the transcription or translation of the nucleic acid encoding the KChIP1 polypeptide; by altering posttranslational processing of the KChIP1 polypeptide; by altering transcription of splicing variants; or by interfering with polypeptide activity (e.g., by binding to the KChIP1 polypeptide, or by binding to another polypeptide that interacts with KChIP1, such as a KChIP1 binding agent as described herein), by altering (e.g., downregulating) the expression, transcription or translation of a nucleic acid encoding KChIP1; or by altering interaction among KChIP1 and a KChIP1 binding agent.

In a further embodiment, the invention relates to Type II diabetes therapeutic agent, such as an agent selected from the group consisting of: a KChIP1 nucleic acid or fragment or derivative thereof; a polypeptide encoded by a KChIP1 nucleic acid (e.g., encoded by a KChIP1 nucleic acid having one or more polymorphism(s) such as those set forth in Table 10); a KChIP1 receptor; a KChIP1 binding agent; a peptidomimetic; a fusion protein; a prodrug; an antibody; an agent that alters KChIP1 gene or nucleic acid expression; an agent that alters activity of a polypeptide encoded by a KChIP1 gene or nucleic acid; an agent that alters posttranscriptional processing of a polypeptide encoded by a KChIP1 gene or nucleic acid; an agent that alters interaction of a KChIP1 polypeptide with a KChIP1 binding agent or receptor; an agent that alters transcription of splicing variants encoded by a KChIP1 gene or nucleic acid; and ribozymes. The invention also relates to pharmaceutical compositions comprising at least one Type II diabetes therapeutic agent as described herein.

The invention also pertains to a method of treating a disease or condition associated with a KChIP1 polypeptide (e.g., Type II diabetes) in an individual, comprising administering a Type II diabetes therapeutic agent to the individual, in a therapeutically effective amount. In certain embodiments, the Type II diabetes therapeutic agent is a KChIP1 agonist; in other embodiments, the Type II diabetes therapeutic agent is a KChIP1 antagonist. The invention additionally pertains to use of a Type II diabetes therapeutic agent as described herein, for the manufacture of a medicament for use in the treatment of Type II diabetes, such as by the methods described herein.

A transgenic animal comprising a nucleic acid selected from the group consisting of: an exogenous KChIP1 gene or nucleic acid and a nucleic acid encoding a KChIP1 polypeptide, is further contemplated by the invention.

In yet another embodiment, the invention relates to a method for assaying a sample for the presence of a KChIP1 nucleic acid, comprising contacting the sample with a nucleic acid comprising a contiguous nucleotide sequence which is at least partially complementary to a part of the sequence of said KChIP1 nucleic acid under conditions appropriate for hybridization, and assessing whether hybridization has occurred between a KChIP1 nucleic acid and said nucleic acid comprising a contiguous nucleotide sequence which is at least partially complementary to a part of the sequence of said KChIP1 nucleic acid; wherein if hybridization has occurred, a KChIP1 nucleic acid is present in sample. In certain embodiments, the contiguous nucleotide sequence is completely complementary to a part of the sequence of said KChIP1 nucleic acid. If desired, amplification of at least part of said KChIP1 nucleic acid can be performed.

In certain other embodiments, the contiguous nucleotide sequence is 100 or fewer nucleotides in length and is either at least 80% identical to a contiguous sequence of nucleotides of one or more of SEQ ID NOs: 1 and 114-258; at least 80% identical to the complement of a contiguous sequence of nucleotides of one or more of SEQ ID NOs: 1 and 114-258; or capable of selectively hybridizing to said KChIP1 nucleic acid.

In other embodiments, the invention relates to a reagent for assaying a sample for the presence of a KChIP1 gene or nucleic acid, the reagent comprising a contiguous nucleotide sequence which is at least partially complementary to a part of the nucleic acid sequence of said KChIP1 gene or nucleic acid; or comprising a contiguous nucleotide sequence which is completely complementary to a part of the nucleic acid sequence of said KChIP1 gene or nucleic acid. Also contemplated by the invention is a reagent kit, e.g., for assaying a sample for the presence of a KChIP1 nucleic acid, comprising (e.g., in separate containers) one or more labeled nucleic acids comprising a contiguous nucleotide sequence which is at least partially complementary to a part of the nucleic acid sequence of the KChIP1 nucleic acid, and reagents for detection of said label. In certain embodiments, the labeled nucleic acid comprises a contiguous nucleotide sequence that is completely complementary to a part of the nucleotide sequence of said KChIP1 gene or nucleic acid. In other embodiments, the labeled nucleic acid can comprise a contiguous nucleotide sequence which is at least partially complementary to a part of the nucleotide sequence of said KChIP1 gene or nucleic acid, and which is capable of acting as a primer for said KChIP1 nucleic acid when maintained under conditions for primer extension.

The invention also provides for the use of a nucleic acid which is 100 or fewer nucleotides in length and which is either: a) at least 80% identical to a contiguous sequence of nucleotides of one or more of SEQ ID NOs: 1 and 114-258; b) at least 80% identical to the complement of a contiguous sequence of nucleotides of one or more of SEQ ID NOs: 1 and 114-258; or c) capable of selectively hybridizing to said KChIP1 nucleic acid, for assaying a sample for the presence of a KChIP1 nucleic acid.

In yet another embodiment, the use of a first nucleic acid which is 100 or fewer nucleotides in length and which is either: a) at least 80% identical to a contiguous sequence of nucleotides of one or more of SEQ ID NOs: 1 and 114-258; b) at least 80% identical to the complement of a contiguous sequence of nucleotides of one or more of SEQ ID NOs: 1 and 114-258; or c) capable of selectively hybridizing to said KChIP1 nucleic acid; for assaying a sample for the presence of a KChIP1 gene or nucleic acid that has at least one nucleotide difference from the first nucleic acid (e.g., a SNP as set forth in Table 10), such as for diagnosing a susceptibility to a disease or condition associated with a KChIP1.

The invention also relates to a method of diagnosing Type II diabetes or a susceptibility to Type II diabetes in an individual, comprising determining the presence or absence in the individual of certain “haplotypes” (combinations of genetic markers). In one aspect of the invention of diagnosising a susceptibility of the disease, methods are described comprising screening for one of the at-risk haplotypes in the KChIP1 gene that is more frequently present in an individual susceptible to Type II diabetes, compared to the frequency of its presence in the general population, wherein the presence of an at-risk haplotype is indicative of a susceptibility to Type II diabetes. An “at-risk haplotype” is intended to embrace one or a combination of haplotypes described herein over the KChIP1 gene that show high correlation to Type II diabetes. In one embodiment, the at-risk haplotype is characterized by the presence of at least one single nucleotide polymorphisms as described in Table 13. In one embodiment, a haplotype associated with Type II diabetes or a susceptibility to Type II diabetes comprises one or more haplotypes identified in Table 2 (haplotypes identified as A1, A2, A3, A4, A5, A6, B1, B2, B3, B4 and B5), Table 4 (haplotypes identified as D1 and D2), Table 5 (haplotypes identified as D2, D3, D4, D5 and D6) Table 14 (haplotypes identified as Hap S7 (formerly E) and Hap S7′ formerly Hap. E′), Table 16 (haplotypes F1, F2 G1 and G2), Table 17 and Table 18 (haplotye S3) or S7. In certain embodiments, a haplotype associated with Type II diabetes or a susceptibility to Type II diabetes comprises markers DG5S879, DG5S881, D5S2075, DG5S883 and DG5S38 at the 5q35 locus; or DG5S1058 and DG5S37 at the 5q35 locus; or DG5S1058, DG5S37 and DG5S101 at the 5q35 locus; or DG5S881, DG5S1058, D5S2075, DG5S883 and DG5S38 at the 5q35 locus; or DG5S879, DG5S1058 and DG5S37; or DG5S881, D5S2075, DG5S883 and DG5S38 at the 5q35 locus; DG5S953, DG5S955, DG5S13 and DG5S959 at the 5q35 locus; or DG5S888 and DG5S953 at the 5q35 locus; or DG5S953, DG5S955 and DG5S124 at the 5q35 locus; or DG5S888, DG5S44 and DG5S953 at the 5q35 locus; DG5S953, DG5S955, DG5S13, DG5S123, and DG5S959 or at SG05S96, DG00AAJIB and DG00AAJHF at the 5q35 locus. The presence of the haplotype is diagnostic of Type II diabetes or of a susceptibility to Type II diabetes. Also described herein is a haplotype associated with Type II diabetes or a susceptibility to Type II diabetes comprising markers DG5S13, KCP_{—}1152, and D5S625 at the 5q35 locus; the presence of the haplotype is diagnostic of Type II diabetes or of a susceptibility to Type II diabetes. In one particular embodiment, the presence of the −4, 1, 0 haplotype at DG5S13, KCP_{—}1152, and D5S625 is diagnostic of Type II diabetes or of a susceptibility to Type II diabetes. In another embodiment, a haplotype associated with Type II diabetes or a susceptibility to Type II diabetes in an individual, comprises markers DG5S124, KCP_{—}1152, KCP_{—}2649, KPC_{—}4976 and KPC-16152 at the 5q35 locus. In one particular embodiment, the presence of the 0, 1, 1, 3 and 0 haplotype at DG5S 124, KCP_{—}1152, KCP_{—}2649, KPC_{—}4976 and KPC-16152 is diagnostic of Type II diabetes or of a susceptibility to Type II diabetes. In another embodiment, a haplotype associated with Type II diabetes or a susceptibility to Type II diabetes in an individual, comprises markers KCP_{—}173982, KCP_{—}15400, and KCP_{—}18069. In one particular embodiment, the presence of the 0, 1, 1 haplotype at KCP_{—}173982, KCP_{—}15400, and KCP_{—}18069 is diagnostic of Type II diabetes or of a susceptibility to Type II diabetes.

In additional embodiments, a haplotype associated with Type II diabetes or a susceptibility to Type II diabetes comprises markers DG5S124, KCP_{—}1152, KCP_{—}2649, KCP_{—}4976, and KCP_{—}16152 at the 5q35 locus, as well as one of the following 3 markers: KCP_{—}197678, KCP_{—}197775, and KCP_{—}202795 at the 5q35 locus; the presence of the haplotype is diagnostic of Type II diabetes or of a susceptibility to Type II diabetes. In particular embodiments, the presence of the 0, 3, 1, 1, 3, 0 haplotype at DG5S124, KCP_{—}197678, KCP_{—}1152, KCP_{—}2649, KCP_{—}4976, and KCP_{—}16152; the presence of the 0, 3, 1, 1, 3, 0 haplotype at DG5S 124, KCP_{—}197775, KCP_{—}1152, KCP_{—}2649, KCP_{—}4976, and KCP_{—}16152; the presence of the 0, 1, 1, 1, 3, 0 haplotype at DG5S124, KCP_{—}202795, KCP_{—}1152, KCP_{—}2649, KCP_{—}4976, and KCP_{—}16152 or the presence of 2,1,1 haplotype at SG05S96, DG00AAJIB and DG00AAJHF; is diagnostic of Type II diabetes or of a susceptibility to Type II diabetes.

In additional embodiments, a haplotype associated with Type II diabetes or a susceptibility to Type II diabetes comprises markers rs1032856, KCP_RS888934, KCP_{—}93545, KCP_{—}102882, 169234, KCP_{—}186048 and KCP_{—}16152, as well as markers rs1032856, KCP_RS888934, KCP_{—}93545, KCP_{—}102882, 169234, KCP_{—}186048, KCP_{—}197775 and KCP_{—}16152 at the 5q35 locus; the presence of the haplotype is diagnostic of Type II diabetes or of a susceptibility to Type II diabetes. In particular embodiments, the presence of the G, G, T, C, G, G, A haplotype at rs1032856, KCP_RS888934, KCP_{—}93545, KCP_{—}102882, 169234, KCP_{—}186048 and KCP_{—}16152, or the presence of the G, G, T, C, G, G, C, A haplotype at rs1032856, KCP_RS888934, KCP_{—}93545, KCP_{—}102882, 169234, KCP_{—}186048, KCP_{—}197775 and KCP_{—}16152 is diagnostic of Type II diabetes or of a susceptibility to Type II diabetes.

In one particular embodiment, the presence of 2,1,1 haplotype at SG05S96, DG00AAJIB and DG00AAJHF is diagnostic of Type II diabetes or of a susceptibility to Type II diabetes. In another embodiment, a haplotype associated with Type II diabetes or a susceptibility to Type II diabetes in an individual, comprises SG05S96, DG00AAJIB and DG00AAJHF.

In another embodiment, a haplotype associated with Type II diabetes or a susceptibility to Type II diabetes in an individual, comprises the presence of G G T C G G A at SG05S96, SG05S93, SG05S64, DG00AAJIB, SG05S156, SG05S213, and SG05S948. In addition embodiments, the additional resequencing exercises can identify significant SNPs, for example, Table 26. These are of the same surrogate class, i.e., they are in perfect linkage disequalibrium with each other.

The presence or absence of the haplotype can be determined by various methods, including, for example, using enzymatic amplification of nucleic acid from the individual, electrophoretic analysis, restriction fragment length polymorphism analysis and/or sequence analysis.

Also described herein is a method of diagnosing Type II diabetes in an individual, comprising determining the presence or absence in the individual of a haplotype comprising one or more markers and/or single nucleotide polymorphisms as shown in Table 10, Table 2, Table 4, Table 5, Tables 13, Table 14, Table 16, Table 17, Table 18, Tables 20-24, and/or Table 26 in the locus on chromosome 5q35, wherein the presence of the haplotype is diagnostic of Type II diabetes. Also contemplated is a method of diagnosing a susceptibility to Type II diabetes in an individual, comprising determining the presence or absence in the individual of a haplotype comprising one or more markers and/or single nucleotide polymorphisms as shown in Table 10, Table 13, Table 14 and/or Table 26 in the locus on chromosome 5q35, wherein the presence of the haplotype is diagnostic of a susceptibility to Type II diabetes.

A method for the diagnosis and identification of a susceptibility to Type II diabetes in an individual is also described, comprising: screening for an at-risk haplotype in the KChIP1 nucleic acid that is more frequently present in an individual susceptible to Type II diabetes compared to an individual who is not susceptible to Type II diabetes, wherein the at-risk haplotype increases the risk significantly. In certain embodiments, the significant increase is at least about 20% or the significant increase is identified as an odds ratio of at least about 1.2.

In another embodiment, the invention features a method of diagnosing a predisposition or susceptibility to Type II diabetes in a subject, comprising detecting the presence or absence of a genetic marker associated with the KChIP1 gene, the marker having a p-value of 1×1-5 or less, wherein the presence of the marker associated with the KChIP1 gene is indicative of a predisposition or susceptibility to Type II diabetes.

In another embodiment, the invention features a method of diagnosing a predisposition or susceptibility to an Type II diabetes associated condition in a subject, comprising detecting the presence or absence of a genetic marker associated with the KChIP1 gene, the marker having a p-value of 1×10⁻⁵ or less, wherein the presence of the marker associated with the KChIP1 gene is indicative of a predisposition or susceptibility to an Type II diabetes associated condition.

In other embodiments, the at-risk haplotype has a relative risk of at least 1.5, at least 2.5 or at least 3.0. In other embodiments, the at-risk haplotype associated with the KChIP1 gene has a p-value of 1×10⁻⁵ or less, 1×10⁻⁶ or less, 1×10⁻⁷ or less or 1×10⁻⁸ or less.

A major application of the current invention involves prediction of those at higher risk of developing a Type II diabetes. Diagnostic tests that define genetic factors contributing to Type II diabetes could be used together with or independent of the known clinical risk factors to define an individual's risk relative to the general population. Better means for identifying those individuals at risk for Type II diabetes should lead to better prophylactic and treatment regimens, including more aggressive management of the current clinical risk factors.

Another application of the current invention is the specific identification of a rate-limiting pathway involved in Type II diabetes. A disease gene with genetic variation that is significantly more common in diabetic patients as compared to controls represents a specifically validated causative step in the pathogenesis of Type II diabetes. That is, the uncertainty about whether a gene is causative or simply reactive to the disease process is eliminated. The protein encoded by the disease gene defines a rate-limiting molecular pathway involved in the biological process of Type II diabetes predisposition. The proteins encoded by such Type II genes or its interacting proteins in its molecular pathway can represent drug targets that may be selectively modulated by small molecule, protein, antibody, or nucleic acid therapies. Such specific information is greatly needed since the population affected with Type II diabetes is growing.

A third application of the current invention is its use to predict an individual's response to a particular drug, even drugs that do not act on KChIP1 or its pathway. It is a well-known phenomenon that in general, patients do not respond equally to the same drug. Much of the differences in drug response to a given drug is thought to be based on genetic and protein differences among individuals in certain genes and their corresponding pathways. The present invention defines the association of KChIP1 with Type II diabetes. Some current or future therapeutic agents may be able to affect this gene directly or indirectly and therefore, be effective in those patients whose Type II diabetes risk is in part determined by the KChIP1 genetic variation. On the other hand, those same drugs may be less effective or ineffective in those patients who do not have at risk variation in the KChIP1 gene. Therefore, KChIP1 variation or haplotypes can be used as a pharmacogenomic diagnostic to predict drug response and guide choice of therapeutic agent in a given individual.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing and other objects, features and advantages of the invention will be apparent from the following more particular description of preferred embodiments of the invention, as illustrated in the accompanying drawings.

FIG. 1A through 1R6 show the KChIP1 genomic DNA (SEQ ID NO: 1). This sequence is taken from NCBI Build 33. The numbering in FIG. 1, as well as the “start” and “end” numbers in all Tables refer to the location in Chromosome 5 in NCBI Build 33 or Build 34. The numbering in FIG. 1 refers to the last base in the line immediately preceding the number; the numbers are in decreasing order because of the “reverse orientation” of the gene.

FIG. 2 shows the amino acid sequence of KChIP1 as published by An et al. Nature, 403(6768): 553-6 (2000) (SEQ ID NO: 2).

FIG. 3 shows the nucleic acid sequence (SEQ ID NO: 3) encoding the amino acid sequence of KChIP1 as published by An et al, Nature, 403(6768): 553-6 (2000) (SEQ ID NO: 2).

FIG. 4 is a series of graphs showing the results of a genome-wide scan using 906 microsatellite markers. Results are shown for three phenotypes: all Type II diabetics (solid lines), obese Type II diabetics (dotted lines) and non-obese Type II diabetics (dashed lines). The multipoint allele-sharing LOD-score is on the vertical axis, and the centimorgan distance from the P-terminus of the chromosome is on the horizontal axis.

FIG. 5 graphically depicts the multipoint allele-sharing LOD-score of the locus on chromosome 5 after 38 microsatellite markers have been added to the framework set in a 40-cM interval, from 160 cM to 200 cM. Results are shown for the same three phenotypes as in FIG. 4; all Type II diabetics (solid line), non-obese Type II diabetics (dashed line) and obese Type II diabetics (dotted line) the results of a genome-wide scan using 906 microsatellite markers.

FIG. 6 graphically depicts the single-marker and haplotype association within the 1-LOD-drop for 590 non-obese diabetics vs 477 unrelated population controls. The location of the markers and haplotypes is on the horizontal axis and the corresponding two-sided P-value on the vertical axis. All haplotypes with a P-value less than 0.01 are shown. The horizontal bars indicate the span of the corresponding haplotypes and the marker density is shown at the bottom of the figure. All locations refer to NCBI Build 33 and the 1-LOD-drop spans from 167.64 to 171.28 Mb.

FIG. 7 schematically shows the location of genes and markers in region B. The microsatellites used in the locus-wide association study are shown as filled circles at the top. The filled boxes indicate the locations of exons, or clusters of exons, for KCHIP1. The shaded boxes indicated the location and size of the neighboring genes, LCP2, KCNMB1, GABRP and RANBP17, and the grey horizontal lines indicate the span of the five most significant microsatellite haplotypes in the region.

FIG. 8A is a plot of the single-marker and haplotype association within the 1-LOD-drop for the genotyped 475 unrelated non-obese Icelandic T2D patients and 477 unrelated population controls. The location of the markers/haplotypes is on the horizontal axis and the corresponding two-sided p-value on the vertical axis. All haplotypes with a p-value less than 0.01 are shown. The horizontal bars indicate the span of the corresponding haplotypes and the marker density is shown at the bottom of the figure. All locations refer to NCBI Build 34. The 3.64 Mb 1-LOD-drop spans between the boundary markers of DG5S5 and D5S429 i.e. 167.68-171.32 Mb.

FIG. 8B schematically shows the location of genes and markers in region B (Build 34). The microsatellites used in locus-wide association study are shown as filled blue circles at the top. The filled red boxes indicate the locations of exons, or clusters of exons, for KChIP1. The shaded green boxes indicated the location and size of the neighboring genes, LCP2, KCNMB1, GABRP and RANBP17, and the grey horizontal lines indicate the span of the haplotypes observed (G1(B1), G2(B2), D1, D2, S3) in the region.

FIG. 8C graphically depicts the KChIP1 region of interest with respect to linkage disequilibrium (LD) of the SNPs identified across the gene (allelic frequency>5%; n=456). The markers are plotted equidistantly rather than according to their physical positions; the relative locations of the KChIP1 exons, haplotypes D1, D2 and S3 are indicated.

FIG. 9A is a multi-tissue Northern blot showing that KChIP1 is expressed in pancreatic β-cells, the blot was hybridized with a human KChIP1 specific probe spanning exons 1 and 2.

FIG. 9B is a Western blot produced with 50 μg of pancreatic tissue lysate or INS-1 cell lysate applied to 4-20% gradient SDS-PAGE and probed with an anti-KChIP1 antiserum (0.5 μg/ml). An anti-GAPDH antibody (1:50 000) was used as control for equal loading. A band of the corresponding size was in brain lysate but not in kidney or pancreas (brain and kidney not shown).

FIG. 10A is a western blot of INS1-shLuc and INS1-shKChIP1#432 cell lysates. Increasing amounts of protein 5, 10 or 25 μg were subjected to western blotting and probed with rabbit anti-KChIP1 (0.5 μg/ml) and mouse anti-GAPDH (1:50000), respectively. The intensity of the bands was quantified with Kodak 1D software and the amount in INS-1 shLuc cells (10 μg) was set to 100%, resulting in a calculated 82% knock down of endogenous KChIP1 expression FIG. 10B graphically depicts the results from an experiment of INS-1 cells either expressing siRNAs for KChIP1 (#sh432) or luciferase when incubated in KRB containing 0.5 mM glucose or induced with 11.2 mM for 2 h. Secreted insulin levels were quantified using an ELISA. The results are expressed as the mean±SEM of the insulin levels measured in 12 independent experiments. A two-sided student's t-test assuming unequal variance was performed. P-values were 1.84×10⁻⁷ (for basal) and p=1.89×10⁻⁶ (for 11.2 mM glucose).

FIG. 11A is a western blot analysis of whole cell lysates from INS-1 cells transduced with an empty retrovirus (control) or a retrovirus expressing the human KChIP1 cDNA. Blots were probed with anti-KChIP1 antiserum (0.5 ug/ml) and subsequently with GAPDH mouse monoclonal antibody (1:50,000).

FIG. 11B graphically depicts the results of INS-1 cells transduced with the control retrovirus (control) or the KChIP1 expressing retroviruses (KChIP1) when incubated in KRB containing 0.5 mM glucose or 11.2 mM glucose for 2 h. The results are expressed as the mean±SEM of the insulin levels measured in 12 independent experiments. The reduction in insulin release between control and KChIP1 cells was significant (two-sided p-values=5.2×10−5).

FIG. 12 graphically depicts the KChIP1 region of interest with respect to linkage disequilibrium (LD) of the 66 SNPs identified across the gene.

FIG. 13A-13E4 show Table 6 that comprises the DNA sequence of the microsatellites employed for the Co5 locus wide association (including Build 33 locations) and Table 7 that comprises the DNA Sequence of microsatellites employed for the association studies across KChIP1 (including Build 33 locations) and Table 10 that comprises DNA sequence of the SNPs identified across KChIP1.

FIG. 14A-14E7 show the SNP amplimer sequences for the study.

### DETAILED DESCRIPTION OF THE INVENTION

Extensive genealogical information for a population with population-based lists of patients with Type II diabetes have been combined with powerful gene sharing methods to map a locus on chromosome 5q35. Diabetics and their relatives were genotyped with a genome-wide marker set including 906 microsatellite markers, with an average marker density of 4 cM. Due to the role obesity plays in the development of diabetes, the material was fractionated according to body mass index (BMI). Presented herein are results of a genome wide search of genes that cause Type II diabetes in Iceland.

Loci Associated with Diabetes

Type 2 diabetes is characterized by hyperglycemia which can occur through one or more physiologic mechanisms including impaired insulin secretion, insulin resistance in peripheral tissues, and increased glucose output by the liver. Most patients suffer the serious complications of chronic hyperglycemia including nephropathy, neuropathy, retinopathy and the accelerated development of cardiovascular disease.

There is strong evidence for a genetic component to the risk of Type II diabetes, including prevalence differences among various racial groups. Mutations in six genes have been discovered that cause MODY, or maturity onset diabetes of the young. MODY1-MODY6 are due to mutations in HNF4a, glucokinase, HNF1a, IPF1, HNF1b and NEUROD1 (MODY1: Yamagata K, et al., Nature 384:458-460 (1996); MODY2: Froguel P, F et al., Nature 356: 162-164 (1992); MODY3: Yamagata, K., et al., Nature 384: 455-458 (1996); MODY4: Yoshioka M., et al., Diabetes, May; 46(5):887-94 (1997) MODY5: Horikawa, Y., et al., Nat. Genet. 17: 384-385 (1997) MODY6: Kristinsson S. Y., et al., Diabetologia, November: 44(11):2098-103 (2001)).

One gene has been identified as a disease gene that contributes to the late-onset form of diabetes, the calpain 10 gene (CAPN10). CAPN10, was identified though a genome-wide screen of Mexican American sibpairs with diabetes (Horikawa, Y., et al., Nat. Genet. 26(2) 163-175 (2000)). The risk allele has been shown to be associated with impaired regulation of glucose-induced secretion and decreased rate of insulin-stimulated glucose disposal (Lynn, S., et al., Diabetes, 51(1): 247-250 (2002); Sreenan, S. K., et al., Diabetes 50(9) 2013-2020 (2001) and Baier, L. J., et al., J. Clin. Invest. 106(7) R69-73 (2000)).

Many genome-wide screens in a variety of populations have been performed that have resulted in major loci for Diabetes. Loci are reported on chromosome 2q37 (Hanis, C. L., et al., Nat. Genet., 13(2):161-166 (1996)), chromosome 15q21 (Cox, et al., Nat. Genet. 21(2):213-215 (1999)), chromosome 10q26 (Duggirala, R., et al., Am. J. Hum. Genet., 68(5):1149-1164 (2001)), chromosome 3p (Ehm, M. G., et al., Am. J. Hum. Genet., 66(6):1871-1881 (2000)) in Mexican Americans, and chromosomes 1q21-23 and 11 q23-q25 (Hanson R. L. et al., Am J. Hum Genet., 63(4):1130-1138 (1998)) in PIMA Indians. In the Caucasian population, linkages have been observed to chromosome 12q24 in Finns (Mahtani, et al., Nat. Genet., 14(1):90-4 (1994)), chromosome 1q21-q23 in Americans in Utah (Elbein, S. C., et al., Diabetes, 48(5): 1175-1182 (1999)), chromosome 3q27-pter in French families (Vionnet, N., et al., Am. J. Hum. Genet. 67(6): 1470-80 (2000) and chromosome 18p11 in Scandinavians (Parker, A., et al., Diabetes, 50(3) 675-680 (2001)). A recent study reported a major locus in indigenous Australians on chromosome 2q24.3 (Busfield, F., et al., Am. J. Hum. Genet., 70(2): 349-357 (2002)). Many other studies have resulted in suggestive loci or have replicated these loci.

Association studies have been reported for Type II diabetes. Most of these studies show modest association to the disease in a group of people but do not account for the disease. Altshuler et al. reviewed the association work that has been done and concluded that association to only one of 16 genes revealed held up to scrutiny. Altshuler et al. confirmed that the Pro12Ala polymorphism in PPARg is associated with Type II diabetes. Until now, there have been no linkage studies in Type II diabetes linking the disease to chromosome 5q35.

Assessment for At-Risk Haplotypes

Populations of individuals exhibiting genetic diversity do not have identical genomes; in other words, there are many polymorphic sites in a population. In some instances, reference is made to different alleles at a polymorphic site without choosing a reference allele. Alternatively, a reference sequence can be referred to for a particular polymorphic site. The reference allele is sometimes referred to as the “wild-type” allele and it usually is chosen as either the first sequenced allele or as the allele from a “non-affected” individual (e.g., an individual that does not display a disease or abnormal phenotype). Alleles that differ from the reference are referred to as “variant” alleles.

A “haplotype,” as described herein, refers to a combination of genetic markers (“alleles”), such as those set forth in Table 2, Table 4, Table 5, Table 14, and Tables 16-18. In a certain embodiment, the haplotype can comprise one or more alleles, two or more alleles, three or more alleles, four or more alleles, or five or more alleles. The genetic markers are particular “alleles” at “polymorphic sites” associated with KChIP1. A nucleotide position at which more than one sequence is possible in a population (either a natural population or a synthetic population, e.g., a library of synthetic molecules) is referred to herein as a “polymorphic site”. Where a polymorphic site is a single nucleotide in length, the site is referred to as a single nucleotide polymorphism (“SNP”). For example, if at a particular chromosomal location, one member of a population has an adenine and another member of the population has a thymine at the same position, then this position is a polymorphic site, and, more specifically, the polymorphic site is a SNP. Polymorphic sites can allow for differences in sequences based on substitutions, insertions or deletions. Each version of the sequence with respect to the polymorphic site is referred to herein as an “allele” of the polymorphic site. Thus, in the previous example, the SNP allows for both an adenine allele and a thymine allele.

Typically, a reference sequence is referred to for a particular sequence. Alleles that differ from the reference are referred to as “variant” alleles. For example, the reference KChIP1 sequence is described herein by SEQ ID NO: 1. The term, “variant KChIP1”, as used herein, refers to a sequence that differs from SEQ ID NO: 1 but is otherwise substantially similar. The genetic markers that make up the haplotypes described herein are KChIP1 variants. Additional variants can include changes that affect a polypeptide, e.g., the KChIP1 polypeptide. These sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence, as described in detail above. Such sequence changes alter the polypeptide encoded by a KChIP1 nucleic acid. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism associated with Type II diabetes or a susceptibility to Type II diabetes can be a synonymous change in one or more nucleotides (i.e., a change that does not result in a change in the amino acid sequence). Such a polymorphism can, for example, alter splice sites, affect the stability or transport of mRNA, or otherwise affect the transcription or translation of the polypeptide. The polypeptide encoded by the reference nucleotide sequence is the “reference” polypeptide with a particular reference amino acid sequence, and polypeptides encoded by variant alleles are referred to as “variant” polypeptides with variant amino acid sequences.

The haplotypes described herein are a combination of various genetic markers, e.g., SNPs and microsatellites. The haplotypes can comprise a combination of various genetic markers, therefore, detecting haplotypes can be accomplished by methods known in the art for detecting sequences at polymorphic sites. For example, standard techniques for genotyping for the presence of SNPs and/or microsatellite markers can be used, such as fluorescent based techniques (Chen, et al., Genome Res. 9, 492 (1999)), PCR, LCR, Nested PCR and other techniques for nucleic acid amplification. These markers and SNPs can be identified in at-risk haplotypes. Certain methods of identifying relevant markers and SNPs include the use of linkage disequilibium (LD) and/or LOD scores.

Linkage Disequilibrium

Linkage Disequilibrium (LD) refers to a non-random assortment of two genetic elements. For example, if a particular genetic element (e.g., “alleles” at a polymorphic site) occurs in a population at a frequency of 0.25 and another occurs at a frequency of 0.25, then the predicted occurrance of a person's having both elements is 0.125, assuming a random distribution of the elements. However, if it is discovered that the two elements occur together at a frequency higher than 0.125, then the elements are said to be in linkage disequilibrium since they tend to be inherited together at a higher rate than what their independent allele frequencies would predict. Roughly speaking, LD is generally correlated with the frequency of recombination events between the two elements. Allele frequencies can be determined in a population by genotyping individuals in a population and determining the occurence of each allele in the population. For populations of diploids, e.g., human populations, individuals will typically have two alleles for each genetic element (e.g., a marker or gene).

Many different measures have been proposed for assessing the strength of linkage disequilibrium (LD). Most capture the strength of association between pairs of biallelic sites. Two important pairwise measures of LD are r² (sometimes denoted Δ²) and |\acute{D}|. Both measures range from 0 (no disequilibrium) to 1 (‘complete’ disequilibrium), but their interpretation is slightly different. |\acute{D}| is defined in such a way that it is equal to 1 if just two or three of the possible haplotypes are present, and it is <1 if all four possible haplotypes are present. So, a value of |\acute{D}| that is <1 indicates that historical recombination has occurred between two sites (recurrent mutation can also cause |\acute{D}| to be <1, but for single nucleotide polymorphisms (SNPs) this is usually regarded as being less likely than recombination). The measure r² represents the statistical correlation between two sites, and takes the value of 1 if only two haplotypes are present. It is arguably the most relevant measure for association mapping, because there is a simple inverse relationship between r² and the sample size required to detect association between susceptibility loci and SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model). Measuring LD across a region is not straightforward, but one approach is to use the measure r, which was developed in population genetics. Roughly speaking, r measures how much recombination would be required under a particular population model to generate the LD that is seen in the data. This type of method can potentially also provide a statistically rigorous approach to the problem of determining whether LD data provide evidence for the presence of recombination hotspots. For the methods described herein, a significant r² value can be 0.3, 0.4, 0.5, 0.6, 0.7, 0.8, 0.9 or 1.0.

Haplotypes and LOD Score Definition of a Susceptibility Locus

In certain embodiments, haplotype analysis involves defining a candidate susceptibility locus using LOD scores. The defined regions are then ultra-fine mapped with microsatellite markers with an average spacing between markers of less than 100 kb. All usable microsatellite markers that are found in public databases and mapped within that region can be used. In addition, microsatellite markers identified within the deCODE genetics sequence assembly of the human genome can be used. The frequencies of haplotypes in the patient and the control groups can be estimated using an expectation-maximization algorithm (Dempster A. et al., 1977. J. R. Stat. Soc. B, 39:1-389). An implementation of this algorithm that can handle missing genotypes and uncertainty with the phase can be used. Under the null hypothesis, the patients and the controls are assumed to have identical frequencies. Using a likelihood approach, an alternative hypothesis is tested, where a candidate at-risk-haplotype, which can include the markers described herein, is allowed to have a higher frequency in patients than controls, while the ratios of the frequencies of other haplotypes are assumed to be the same in both groups. Likelihoods are maximized separately under both hypotheses and a corresponding 1-df likelihood ratio statistic is used to evaluate the statistic significance.

To look for at-risk-haplotypes in the 1-lod drop, for example, association of all possible combinations of genotyped markers is studied, provided those markers span a practical region. The combined patient and control groups can be randomly divided into two sets, equal in size to the original group of patients and controls. The haplotype analysis is then repeated and the most significant p-value registered is determined. This randomization scheme can be repeated, for example, over 100 times to construct an empirical distribution of p-values. In a preferred embodiment, a p-value of <0.05 is indicative of an at-risk haplotype.

A detailed discussion of haplotype analysis follows.

Haplotype Analysis

One general approach to haplotype analysis involves using likelihood-based inference applied to NEsted MOdels. The method is implemented in our program NEMO, which allows for many polymorphic markers, SNPs and microsatellites. The method and software are specifically designed for case-control studies where the purpose is to identify haplotype groups that confer different risks. It is also a tool for studying LD structures.

When investigating haplotypes constructed from many markers, apart from looking at each haplotype individually, meaningful summaries often require putting haplotypes into groups. A particular partition of the haplotype space is a model that assumes haplotypes within a group have the same risk, while haplotypes in different groups can have different risks. Two models/partitions are nested when one, the alternative model, is a finer partition compared to the other, the null model, i.e, the alternative model allows some haplotypes assumed to have the same risk in the null model to have different risks. The models are nested in the classical sense that the null model is a special case of the alternative model. Hence traditional generalized likelihood ratio tests can be used to test the null model against the alternative model. Note that, with a multiplicative model, if haplotypes hᵢ and hₑ are assumed to have the same risk, it corresponds to assuming that fᵢ/pᵢ=fⱼ/pⱼ where f and p denote haplotype frequencies in the affected population and the control population respectively.

One common way to handle uncertainty in phase and missing genotypes is a two-step method of first estimating haplotype counts and then treating the estimated counts as the exact counts, a method that can sometimes be problematic (e.g., see the information measure section below) and may require randomization to properly evaluate statistical significance. In NEMO, maximum likelihood estimates, likelihood ratios and p-values are calculated directly, with the aid of the EM algorithm, for the observed data treating it as a missing-data problem.

NEMO allows complete flexibility for partitions. For example, the first haplotype problem described in the Methods section on Statistical analysis considers testing whether h₁ has the same risk as the other haplotypes h₂, . . . , hₖ. Here the alternative grouping is [h₁], [h₂, . . . , hₖ] and the null grouping is [h₁, . . . , hₖ]. The second haplotype problem in the same section involves three haplotypes h₁=G0, h₂=GX and h₃=AX, and the focus is on comparing h₁ and h₂. The alternative grouping is [h₁], [h₂], [h₃] and the null grouping is [h₁, h₂], [h₃]. If composite alleles exist, one could collapse these alleles into one at the data processing stage, and performed the test as described. This is a perfectly valid approach, and indeed, whether we collapse or not makes no difference if there were no missing information regarding phase. But, with the actual data, if each of the alleles making up a composite correlates differently with the SNP alleles, this will provide some partial information on phase. Collapsing at the data processing stage will unnecessarily increase the amount of missing information. A nested-models/partition framework can be used in this scenario. Let h₂ be split into h₂ₐ, h_{2b}, . . . , h₂ₑ, and h₃ be split into h₃ₐ, h_{3b}, . . . , h₃ₑ. Then the alternative grouping is [h₁], [h₂ₐ, h_{2b}, . . . , h₂ₑ], [h₃ₐ, h_{3b}, . . . , h₃ₑ] and the null grouping is [h₁, h₂ₐ, h_{2b}, . . . , h₂ₑ], [h₃ₐ, h_{3b}, . . . , h₃ₑ]. The same method can be used to handle composite where collapsing at the data processing stage is not even an option since L_{C} represents multiple haplotypes constructed from multiple SNPs. Alternatively, a 3-way test with the alternative grouping of [h₁], [h₂ₐ, h_{2b}, . . . , h₂ₑ], [h₃ₐ, h_{3b}, . . . , h₃ₑ] versus the null grouping of [h₁, h₂ₐ, h_{2b}, . . . , h₂ₑ, h₃ₐ, h_{3b}, . . . , h₃ₑ] could also be performed. Note that the generalized likelihood ratio test-statistic would have two degrees of freedom instead of one.

Measuring Information

Even though likelihood ratio tests based on likelihoods computed directly for the observed data, which have captured the information loss due to uncertainty in phase and missing genotypes, can be relied on to give valid p-values, it would still be of interest to know how much information had been lost due to the information being incomplete. Interestingly, one can measure information loss by considering a two-step procedure to evaluating statistical significance that appears natural but happens to be systematically anti-conservative. Suppose we calculate the maximum likelihood estimates for the population haplotype frequencies calculated under the alternative hypothesis that there are differences between the affected population and control population, and use these frequency estimates as estimates of the observed frequencies of haplotype counts in the affected sample and in the control sample. Suppose we then perform a likelihood ratio test treating these estimated haplotype counts as though they are the actual counts. We could also perform a Fisher's exact test, but we would then need to round off these estimated counts since they are in general non-integers. This test will in general be anti-conservative because treating the estimated counts as if they were exact counts ignores the uncertainty with the counts, overestimates the effective sample size and underestimates the sampling variation. It means that the chi-square likelihood-ratio test statistic calculated this way, denoted by Λ*, will in general be bigger than A, the likelihood-ratio test-statistic calculated directly from the observed data as described in methods. But Λ* is useful because the ratio Λ/Λ* happens to be a good measure of information, or 1−(Λ/Λ*) is a measure of the fraction of information lost due to missing information. This information measure for haplotype analysis is described in Nicolae and Kong, Technical Report 537, Department of Statistics, University of Statistics, University of Chicago, Revised for Biometrics (2003) as a natural extension of information measures defined for linkage analysis, and is implemented in NEMO.

Statistical Analysis.

For single marker association to the disease, the Fisher exact test can be used to calculate two-sided p-values for each individual allele. All p-values are presented unadjusted for multiple comparisons unless specifically indicated. The presented frequencies (for microsatellites, SNPs and haplotypes) are allelic frequencies as opposed to carrier frequencies. To minimize any bias due the relatedness of the patients who were recruited as families for the linkage analysis, first and second-degree relatives can be eliminated from the patient list. Furthermore, the test can be repeated for association correcting for any remaining relatedness among the patients, by extending a variance adjustment procedure described in Risch, N. & Teng, J. (Genome Res., 8:1278-1288 (1998)). The relative power of family-based and case-control designs for linkage disequilibrium studies of complex human diseases I. DNA pooling (ibid) for sibships so that it can be applied to general familial relationships, and present both adjusted and unadjusted p-values for comparison. The differences are in general very small as expected. To assess the significance of single-marker association corrected for multiple testing we carried out a randomisation test using the same genotype data. Cohorts of patients and controls can be randomized and the association analysis redone multiple times (e.g., up to 500,000 times) and the p-value is the fraction of replications that produced a p-value for some marker allele that is lower than or equal to the p-value we observed using the original patient and control cohorts.

For both single-marker and haplotype analyses, relative risk (RR) and the population attributable risk (PAR) can be calculated assuming a multiplicative model (haplotype relative risk model), (Terwilliger, J. D. & Ott, J., Hum Hered, 42, 337-46 (1992) and Falk, C. T. & Rubinstein, P, Ann Hum Genet 51 (Pt 3), 227-33 (1987)), i.e., that the risks of the two alleles/haplotypes a person carries multiply. For example, if RR is the risk of A relative to a, then the risk of a person homozygote AA will be RR times that of a heterozygote Aa and RR² times that of a homozygote aa. The multiplicative model has a nice property that simplifies analysis and computations—haplotypes are independent, i.e., in Hardy-Weinberg equilibrium, within the affected population as well as within the control population. As a consequence, haplotype counts of the affecteds and controls each have multinomial distributions, but with different haplotype frequencies under the alternative hypothesis. Specifically, for two haplotypes hᵢ and hⱼ, risk(hᵢ)/risk(hⱼ)=(fᵢ/pᵢ)/(fⱼ/pⱼ), where f and p denote respectively frequencies in the affected population and in the control population. While there is some power loss if the true model is not multiplicative, the loss tends to be mild except for extreme cases. Most importantly, p-values are always valid since they are computed with respect to null hypothesis.

In general, haplotype frequencies are estimated by maximum likelihood and tests of differences between cases and controls are performed using a generalized likelihood ratio test (Rice, J. A. Mathematical Statistics and Data Analysis, 602 (International Thomson Publishing, (1995)). deCODE's haplotype analysis program called NEMO, which stands for NEsted MOdels, can be used to calculate all the haplotype results. To handle uncertainties with phase and missing genotypes, it is emphasized that we do not use a common two-step approach to association tests, where haplotype counts are first estimated, possibly with the use of the EM algorithm, Dempster, (A. P., Laird, N. M. & Rubin, D. B., Journal of the Royal Statistical Society B, 39, 1-38 (1971)) and then tests are performed treating the estimated counts as though they are true counts, a method that can sometimes be problematic and may require randomisation to properly evaluate statistical significance. Instead, with NEMO, maximum likelihood estimates, likelihood ratios and p-values are computed with the aid of the EM-algorithm directly for the observed data, and hence the loss of information due to uncertainty with phase and missing genotypes is automatically captured by the likelihood ratios. Even so, it is of interest to know how much information is retained, or lost, due to incomplete information. Described herein is such a measure that is natural under the likelihood framework. For a fixed set of markers, the simplest tests performed compare one selected haplotype against all the others. Call the selected haplotype h₁ and the others h₂, . . . , hₖ. Let p₁, . . . , pₖ denote the population frequencies of the haplotypes in the controls, and f₁, . . . , fₖ denote the population frequencies of the haplotypes in the affecteds. Under the null hypothesis, fᵢ=pᵢ for all i. The alternative model we use for the test assumes h₂, . . . , hₖ to have the same risk while h₁ is allowed to have a different risk. This implies that while p₁ can be different from f₁,fᵢ/(f₂+ . . . +fₖ)=pᵢ/(p₂+ . . . +pₖ)=βᵢ for i=2, . . . , k. Denoting f₁/p₁ by r, and noting that β₂+ . . . +βₖ=1, the test statistic based on generalized likelihood ratios is
Λ=2[l(\hat{r}, \hat{p}₁, \hat{β}₂, . . . , \hat{β}ₖ₋₁)−l(1,\tilde{p}₁, \tilde{β}₂, . . . , \tilde{β}ₖ₋₁)]
where l denotes logₑlikelihood and ~ and \hat{ } denote maximum likelihood estimates under the null hypothesis and alternative hypothesis respectively. A has asymptotically a chi-square distribution with 1-df, under the null hypothesis. Slightly more complicated null and alternative hypotheses can also be used. For example, let h₁ be G0, h₂ be GX and h₃ be AX. When comparing G0 against GX, i.e., this is the test which gives estimated RR of 1.46 and p-value=0.0002, the null assumes G0 and GX have the same risk but AX is allowed to have a different risk. The alternative hypothesis allows, for example, three haplotype groups to have different risks. This implies that, under the null hypothesis, there is a constraint that f₁/p₁=f₂/p₂, or w=[f₁/p₁]/[f₂/p₂]=1. The test statistic based on generalized likelihood ratios is
Λ=2[l(\hat{p}₁, \hat{f}₁, \hat{p}₂, ŵ)−l(\tilde{p}₁, \tilde{f}₁, \tilde{p}₂, 1)]
that again has asymptotically a chi-square distribution with 1-df under the null hypothesis. If there are composite haplotypes (for example, h₂ and h₃), that is handled in a natural manner under the nested models framework.
Linkage Disequilibrium Using NEMO

LD between pairs of SNPs can be calculated using the standard definition of D′ and R² (Lewontin, R., Genetics 49, 49-67 (1964) and Hill, W. G. & Robertson, A. Theor. Appl. Genet. 22, 226-231 (1968)). Using NEMO, frequencies of the two marker allele combinations are estimated by maximum likelihood and deviation from linkage equilibrium is evaluated by a likelihood ratio test. The definitions of D′ and R² are extended to include microsatellites by averaging over the values for all possible allele combination of the two markers weighted by the marginal allele probabilities. When plotting all marker combination to elucidate the LD structure in a particular region, we plot D′ in the upper left corner and the p-value in the lower right corner. In the LD plots the markers can be plotted equidistant rather than according to their physical location, if desired.

Statistical Methods for Linkage Analysis

Multipoint, affected-only allele-sharing methods can be used in the analyses to assess evidence for linkage. Results, both the LOD-score and the non-parametric linkage (NPL) score, can be obtained using the program Allegro (Gudbjartsson et al., Nat. Genet. 25:12-3, 2000). Our baseline linkage analysis uses the Spairs scoring function (Whittemore, A. S., Halpern, J. (1994), Biometrics 50:118-27; Kruglyak L, et al. (1996), Am J Hum Genet 58:1347-63), the exponential allele-sharing model (Kong, A. and Cox, N. J. (1997), Am J Hum Genet 61:1179-88) and a family weighting scheme that is halfway, on the log-scale, between weighting each affected pair equally and weighting each family equally. The information measure we use is part of the Allegro program output and the information value equals zero if the marker genotypes are completely uninformative and equals one if the genotypes determine the exact amount of allele sharing by decent among the affected relatives (Gretarsdottir et al., Am. J. Hom. Genet, 70:593-603, (2002)). The P-values were computed two different ways and the less significant result is reported here. The first P-value can be computed on the basis of large sample theory; the distribution of Zₗᵣ=√(2 [logₑ(10)LOD]) approximates a standard normal variable under the null hypothesis of no linkage (Kong, A. and Cox, N. J. (1997), Am J Hum Genet 61:1179-88). The second P-value can be calculated by comparing the observed LOD-score with its complete data sampling distribution under the null hypothesis (e.g., Gudbjartsson et al., Nat. Genet. 25:12-3, 2000). When the data consist of more than a few families, these two P-values tend to be very similar.

Haplotypes and “Haplotype Block” Definition of a Susceptibility Locus

In certain embodiments, haplotype analysis involves defining a candidate susceptibility locus based on “haplotype blocks.” It has been reported that portions of the human genome can be broken into series of discrete haplotype blocks containing a few common haplotypes; for these blocks, linkage disequilibrium data provided little evidence indicating recombination (see, e.g., Wall., J. D. and Pritchard, J. K., Nature Reviews Genetics 4: 587-597 (2003); Daly, M. et al., Nature Genet. 29:229-232 (2001); Gabriel, S. B. et al., Science 296:2225-2229 (2002); Patil, N. et al., Science 294:1719-1723 (2001); Dawson, E. et al., Nature 418:544-548 (2002); Phillips, M. S. et al., Nature Genet. 33:382-387 (2003)).

There are two main methods for defining these haplotype blocks: blocks can be defined as regions of DNA that have limited haplotype diversity (see, e.g., Daly, M. et al., Nature Genet. 29:229-232 (2001); Patil, N. et al., Science 294:1719-1723 (2001); Dawson, E. et al., Nature 418:544-548 (2002); Zhang, K. et al., PNAS USA 99:7335-7339 (2002)), or as regions between transition zones having extensive historical recombination, identified using linkage disequilibrium (see, e.g., Gabriel, S. B. et al., Science 296:2225-2229 (2002); Phillips, M. S. et al., Nature Genet. 33:382-387 (2003); Wang, N. et al., Am. J. Hum. Genet. 71:1227-1234 (2002); Stumpf, M. P., and Goldstein, D. B., Curr. Biol. 13:1-8 (2003)). As used herein, the term, “haplotype block” includes blocks defined by either characteristic.

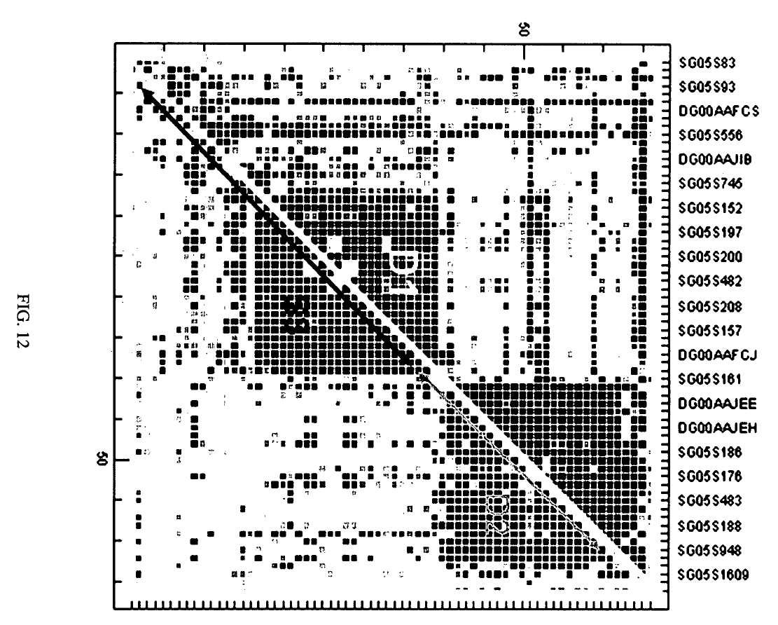

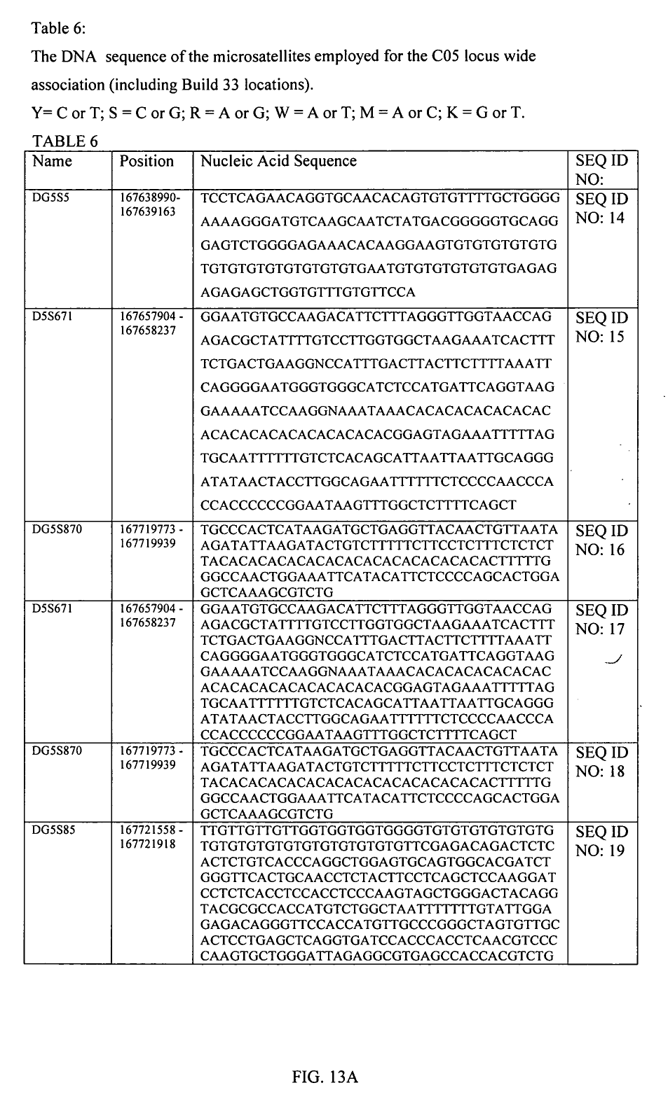

Representative methods for identification of haplotype blocks are set forth, for example, in U.S. Published Patent Applications 20030099964; 20030170665; 20040023237; 20040146870. Haplotype blocks can be used readily to map associations between phenotype and haplotype status. The main haplotytpes can be identified in each haplotype block, and then a set of “tagging” SNPs or markers (the smallest set of SNPs or markers needed to distinguish among the haplotypes) can then be identified. These tagging SNPs or markers can then be used in assessment of samples from groups of individuals, in order to identify association between phenotype and haplotype. If desired, neighboring haplotype blocks can be assessed concurrently, as there may also exist linkage disequilibrium among the haplotype blocks. In the instant invention, one such exemplary block is utilized (FIG. 12), wherein a region associated with KChIP1 is scanned for markers and haplotypes associated with Type II diabetes. Other blocks would be apparent to one of skill in the art as genetic regions in LD with KChIP1. Markers and haplotypes identified in these blocks, because of their association with KChIP1, are encompassed by the invention.

Haplotypes and Diagnostics

Certain haplotypes as described herein, e.g., having markers such as those shown in Table 2, Table 4, Table 5, Table 6, Table 13, Table 14, Table 16, Table 17 and Table 18 are found more frequently in individuals with Type II diabetes than in individuals without Type II diabetes. Therefore, these haplotypes have predictive value for detecting Type II diabetes or a susceptibility to Type II diabetes in an individual. In addition, haplotype blocks comprising certain tagging markers, can be found more frequently in individuals with Type II diabetes than in individuals without Type II diabetes. Therefore, these “at-risk” tagging markers within the haplotype blocks also have predictive value for detecting a susceptibility to Type II diabetes in an individual. “At-risk” tagging markers within the haplotype blocks can also include other markers that distinguish among the haplotypes, as these similarly have predictive value for detecting a susceptibility to Type II Diabetes.

The haplotypes and tagging markers useful herein are in some cases a combination of various genetic markers, e.g., SNPs and microsatellites. Therefore, detecting haplotypes can be accomplished by methods known in the art for detecting sequences at polymorphic sites, such as the methods described above. Furthermore, correlation between certain haplotypes or sets of tagging markers and disease phenotype can be verified using standard techniques. A representative example of a simple test for correlation would be a Fisher-exact test on a two by two table.

In specific embodiments, an at-risk haplotype in, or comprising portions of, the KChIP1 gene, is one where the haplotype is more frequently present in an individual at risk for Type II diabetes (affected), compared to the frequency of its presence in a healthy individual (control), and wherein the presence of the haplotype is indicative of Type II diabetes or susceptibility to Type II diabetes. In other embodiments, at risk tagging markers in a haplotype block in linkage disequilibrium with one or more markers in the KChIP1 gene, are tagging markers that are more frequently present in an individual at risk for Type II diabetes (affected), compared to the frequency of their presence in a healthy individual (control), and wherein the presence of the tagging markers is indicative of susceptibility to Type II diabetes. In a further embodiment, at-risk marks in linkage disequilibrium with one or more markers in the KChIP1 gene, are makers that are more frequently present in an individual at risk for Type II diabetes, compared to the frequency of their presence in a healthy individual (control), and wherein the presence of the markers is indicative of susceptibility to Type II diabetes. In particularly preferred embodiments of the invention, at-risk haplotypes include haplotypes as shown in Table 2, Table 4, Table 5, Table 6, Table 7, Table 9, Tables 11-14, Tables 16-18 and Tables 20-24.

In certain methods described herein, an individual who is at risk for Type II diabetes is an individual in whom an at-risk haplotype is identified, or an individual in whom at-risk tagging markers are identified. In one embodiment, significance associated with a haplotype is measured by an odds ratio. In a further embodiment, the significance is measured by a percentage. In one embodiment, a significant risk is measured as an odds ratio of at least about 1.2, including but not limited to: 1.2, 1.3, 1.4, 1.5, 1.6, 1.7, 1.8 and 1.9. In a further embodiment, an odds ratio of at least 1.2 is significant. In a further embodiment, an odds ratio of at least about 1.5 is significant. In a further embodiment, a significant increase in risk is at least about 1.7 is significant. In a further embodiment, a significant increase in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase in risk is at least about 50%. It is understood however, that identifying whether a risk is medically significant may also depend on a variety of factors, including the specific disease, the haplotype, and often, environmental factors.

Particular embodiments of the invention encompass methods including a method of diagnosing a susceptibility to Type II diabetes in an individual, comprising assessing in an individual the presence or frequency of SNPs and/or microsatellites in, comprising portions of, the KChIP1 gene, wherein an excess or higher frequency of the SNPs and/or microsatellites compared to a healthy control individual is indicative that the individual has Type II diabetes, or is susceptible to Type II diabetes. See, for example, Table 2, Table 4, Table 5, Table 6, Table 7, Table 9, Tables 11-14, Tables 16-18, Tables 20-24 and Table 26 (below) for SNPs and markers that can form haplotypes that can be used as screening tools. These markers and SNPs can be identified in at-risk haplotypes. For example, an at-risk haplotype can include microsatellite markers and/or SNPs such as those set forth in Table 2, Table 4, Table 5, Table 14, Table 16-18 and Table 26. The presence of the haplotype is indicative a susceptibility to Type II diabetes, and therefore is indicative of an individual who falls within a target population for the treatment methods described herein. These haplotypes can be used as screening tools. Other particular embodiments of the invention encompass methods of diagnosing a susceptibility to Type II diabetes in an individual, comprising detecting one or more markers at one or more polymorphic sites, wherein the one or more polymorphic sites are in linkage disequilibium with KChIP1 gene.

Individuals who have been identified as being susceptible to Type II diabetes using the methods described herein are individuals who fall within a target population for the methods of therapy described herein.

Nucleic Acid Therapeutic Agents

In another embodiment, a nucleic acid of the invention; a nucleic acid complementary to a nucleic acid of the invention; or a portion of such a nucleic acid (e.g., an oligonucleotide as described below); or a nucleic acid encoding a KChIP1 polypeptide, can be used in “antisense” therapy, in which a nucleic acid (e.g., an oligonucleotide) which specifically hybridizes to the mRNA and/or genomic DNA of a nucleic acid is administered or generated in situ. The antisense nucleic acid that specifically hybridizes to the mRNA and/or DNA inhibits expression of the polypeptide encoded by that mRNA and/or DNA, e.g., by inhibiting translation and/or transcription. Binding of the antisense nucleic acid can be by conventional base pair complementarity, or, for example, in the case of binding to DNA duplexes, through specific interaction in the major groove of the double helix.

An antisense construct can be delivered, for example, as an expression plasmid as described above. When the plasmid is transcribed in the cell, it produces RNA that is complementary to a portion of the mRNA and/or DNA that encodes a KChIP1 polypeptide. Alternatively, the antisense construct can be an oligonucleotide probe that is generated ex vivo and introduced into cells; it then inhibits expression by hybridizing with the mRNA and/or genomic DNA of the polypeptide. In one embodiment, the oligonucleotide probes are modified oligonucleotides that are resistant to endogenous nucleases, e.g., exonucleases and/or endonucleases, thereby rendering them stable in vivo. Exemplary nucleic acid molecules for use as antisense oligonucleotides are phosphoramidate, phosphothioate and methylphosphonate analogs of DNA (see also U.S. Pat. Nos. 5,176,996, 5,264,564 and 5,256,775). Additionally, general approaches to constructing oligomers useful in antisense therapy are also described, for example, by Van der Krol et al. (BioTechniques 6:958-976 (1988)); and Stein et al. (Cancer Res. 48:2659-2668 (1988)). With respect to antisense DNA, oligodeoxyribonucleotides derived from the translation initiation site are preferred.

To perform antisense therapy, oligonucleotides (mRNA, cDNA or DNA) are designed that are complementary to mRNA encoding the polypeptide. The antisense oligonucleotides bind to mRNA transcripts and prevent translation. Absolute complementarity, although preferred, is not required. A sequence “complementary” to a portion of an RNA, as referred to herein, indicates that a sequence has sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid, as described in detail above. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures.

The oligonucleotides used in antisense therapy can be DNA, RNA, or chimeric mixtures or derivatives or modified versions thereof, single-stranded or double-stranded. The oligonucleotides can be modified at the base moiety, sugar moiety, or phosphate backbone, for example, to improve stability of the molecule, hybridization, etc. The oligonucleotides can include other appended groups such as peptides (e.g., for targeting host cell receptors in vivo), or agents facilitating transport across the cell membrane (see, e.g., Letsinger et al., Proc. Natl. Acad. Sci. USA 86:6553-6556 (1989); Lemaitre et al., Proc. Natl. Acad. Sci. USA 84:648-652 (1987); PCT International Publication NO: WO 88/09810) or the blood-brain barrier (see, e.g., PCT International Publication NO: WO 89/10134), or hybridization-triggered cleavage agents (see, e.g., Krol et al., BioTechniques 6:958-976 (1988)) or intercalating agents. (See, e.g., Zon, Pharm. Res. 5: 539-549 (1988)). To this end, the oligonucleotide may be conjugated to another molecule (e.g., a peptide, hybridization triggered cross-linking agent, transport agent, hybridization-triggered cleavage agent).

The antisense molecules are delivered to cells that express a KChIP1 polypeptide in vivo. A number of methods can be used for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically. Alternatively, in a another embodiment, a recombinant DNA construct is utilized in which the antisense oligonucleotide is placed under the control of a strong promoter (e.g. pol III or pol II). The use of such a construct to transfect target cells in the patient results in the transcription of sufficient amounts of single stranded RNAs that will form complementary base pairs with the endogenous transcripts and thereby prevent translation of the mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art and described above. For example, a plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systemically).

In another embodiment of the invention, small double-stranded interfering RNA (RNA interference (RNAi)) can be used. RNAi is a post-transcription process, in which double-stranded RNA is introduced and sequence-specific gene silencing results, though catalytic degradation of the targeted mRNA. See, e.g., Elbashir, S. M. et al., Nature 411:494-498 (2001); Lee, N. S., Nature Biotech. 19:500-505 (2002); Lee, S-K. et al., Nature Medicine 8(7):681-686 (2002); the entire teachings of these references are incorporated herein by reference. RNAi is used routinely to investigate gene function in a high throughput fashion or to modulate gene expression in human diseases (Chi et al., PNAS, 100 (11):6343-6346 (2003)).

Introduction of long double stranded RNA leads to sequence-specific degradation of homologous gene transcripts. The long double stranded RNA is metabolized to small 21-23 nucleotide siRNA (small interfering RNA). The siRNA then binds to protein complex RISC(RNA-induced silencing complex) with dual function helicase. The helicase has RNAase activity and is able to unwind the RNA. The unwound si RNA allows an antisense strand to bind to a target. This results in sequence dependent degradation of cognate mRNA. Aside from endogenous RNAi, exogenous RNAi, chemically synthesized or recombinantly produced can also be used.

Using non-intronic portions of the KChIP1 gene such as corresponding mRNA portions of SEQ ID NO: 1, target regions of the KChIP1 gene that are accessible for RNAi are targeted and silenced. With this technique it is possible to conduct a RNAi gene walk of the nucleic acids of KChIP1 and determine the amount of inhibition of the protein product. Thus, it is possible to design gene-specific therapeutics by directly targeting the mRNAs of Type II diabetes-related KChIP1 gene.

Endogenous expression of a gene product can also be reduced by inactivating or “knocking out” the gene or its promoter using targeted homologous recombination (e.g., see Smithies et al., Nature 317:230-234 (1985); Thomas & Capecchi, Cell 51:503-512 (1987); Thompson et al., Cell 5:313-321 (1989)). For example, an altered, non-functional gene (or a completely unrelated DNA sequence) flanked by DNA homologous to the endogenous gene (either the coding regions or regulatory regions of the gene) can be used, with or without a selectable marker and/or a negative selectable marker, to transfect cells that express the gene in vivo. Insertion of the DNA construct, via targeted homologous recombination, results in inactivation of the gene. The recombinant DNA constructs can be directly administered or targeted to the required site in vivo using appropriate vectors, as described above. Alternatively, expression of non-altered genes can be increased using a similar method: targeted homologous recombination can be used to insert a DNA construct comprising a non-altered functional gene, or the complement thereof, or a portion thereof, in place of an gene in the cell, as described above. In another embodiment, targeted homologous recombination can be used to insert a DNA construct comprising a nucleic acid that encodes a polypeptide variant that differs from that present in the cell.

Alternatively, endogenous expression of a gene product can be reduced by targeting deoxyribonucleotide sequences complementary to the regulatory region (i.e., the promoter and/or enhancers) to form triple helical structures that prevent transcription of the gene in target cells in the body. (See generally, Helene, C., Anticancer Drug Des., 6(6):569-84 (1991); Helene, C. et al., Ann. N.Y. Acad. Sci. 660:27-36 (1992); and Maher, L. J., Bioassays 14(12):807-15 (1992)). Likewise, the antisense constructs described herein, by antagonizing the normal biological activity of the gene product, can be used in the manipulation of tissue, e.g., tissue differentiation, both in vivo and for ex vivo tissue cultures. Furthermore, the anti-sense techniques (e.g., microinjection of antisense molecules, or transfection with plasmids whose transcripts are anti-sense with regard to a nucleic acid RNA or nucleic acid sequence) can be used to investigate the role of one or more members of the KChIP1 pathway in the development of disease-related conditions. Such techniques can be utilized in cell culture, but can also be used in the creation of transgenic animals.

The therapeutic agents as described herein can be delivered in a composition, as described above, or alone. They can be administered systemically, or can be targeted to a particular tissue. The therapeutic agents can be produced by a variety of means, including chemical synthesis; recombinant production; in vivo production (e.g., a transgenic animal, such as U.S. Pat. No. 4,873,316 to Meade et al.), for example, and can be isolated using standard means such as those described herein. In addition, a combination of any of the above methods of treatment (e.g., administration of non-altered polypeptide in conjunction with antisense therapy targeting altered mRNA; administration of a first splicing variant in conjunction with antisense therapy targeting a second splicing variant) can also be used.

The invention additionally pertains to use of such therapeutic agents, as described herein, for the manufacture of a medicament for the treatment of Type II diabetes e.g., using the methods described herein.

Monitoring Progress of Treatment

The current invention also pertains to methods of monitoring the effectiveness of treatment on the regulation of expression (e.g., relative or absolute expression) of one or more KChIP1 isoforms at the RNA or protein level or its enzymatic activity. KChIP1 message or protein or enzymatic activity can be measured in a sample of peripheral blood or cells derived therefrom. An assessment of the levels of expression or activity can be made before and during treatment with KChIP1 therapeutic agents. For example, in one embodiment of the invention, an individual who is a member of the target population can be assessed for response to treatment with a KChIP1 inhibitor, by examining calcium levels or Kv channel-interacting proteins activity or absolute and/or relative levels of KChIP1 protein or mRNA isoforms in peripheral blood in general or specific cell subfractions or combination of cell subfractions. In addition, variation such as haplotypes or mutations within or near (within 100 to 200 kb) of the KChIP1 gene may be used to identify individuals who are at higher risk for Type II diabetes to increase the power and efficiency of clinical trials for pharmaceutical agents to prevent or treat Type II diabetes. The haplotypes and other variations may be used to exclude or fractionate patients in a clinical trial who are likely to have non-KChIP1 involvement in their Type II diabetes risk in order to enrich patients who have other genes or pathways involved and boost the power and sensitivity of the clinical trial. Such variation may be used as a pharmacogenomic test to guide selection of pharmaceutical agents for individuals.

Described herein is the first known linkage study of Type II diabetes showing a connection to chromosome 5q35. Based on the linkage studies conducted, a direct relationship between Type II diabetes and the locus on chromosome 5q35, in particular the KChIP1 gene, has been discovered.

Nucleic Acids of the Invention

KChIP1 Nucleic Acids, Portions and Variants

Accordingly, the invention pertains to isolated nucleic acid molecules comprising human KChIP1 nucleic acid. The term, “KChIP1 nucleic acid,” as used herein, refers to an isolated nucleic acid molecule encoding a KChIP1 polypeptide (e.g., a KChIP1 gene, such as shown in SEQ ID NO:1). The KChIP1 nucleic acid molecules of the present invention can be RNA, for example, mRNA, or DNA, such as cDNA and genomic DNA. DNA molecules can be double-stranded or single-stranded; single stranded RNA or DNA can be either the coding, or sense, strand or the non-coding, or antisense strand. The nucleic acid molecule can include all or a portion of the coding sequence of the gene and can further comprise additional non-coding sequences such as introns and non-coding 3′ and 5′ sequences (including regulatory sequences, for example).

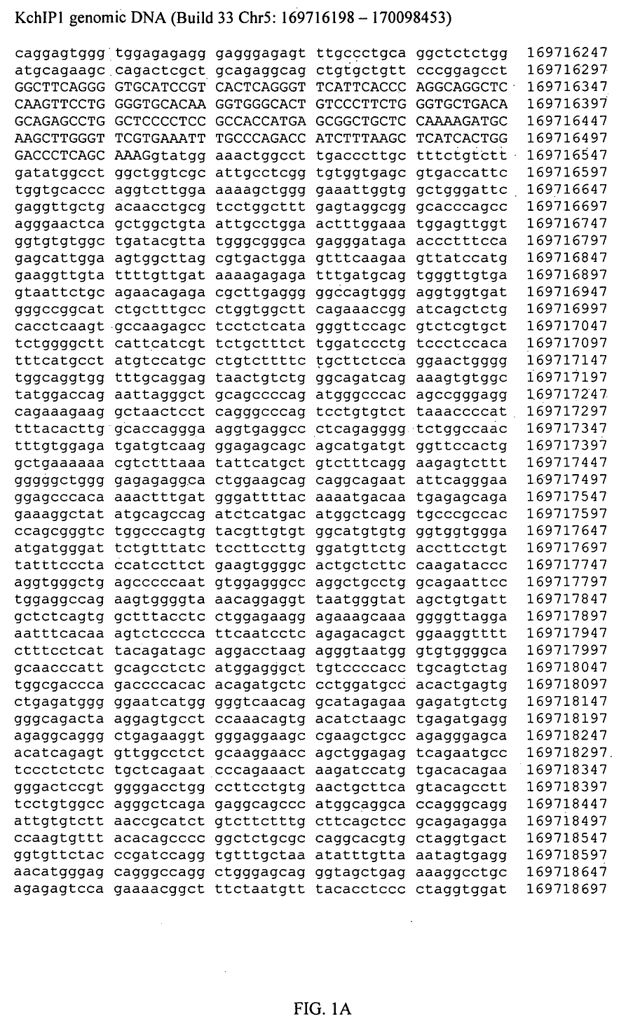

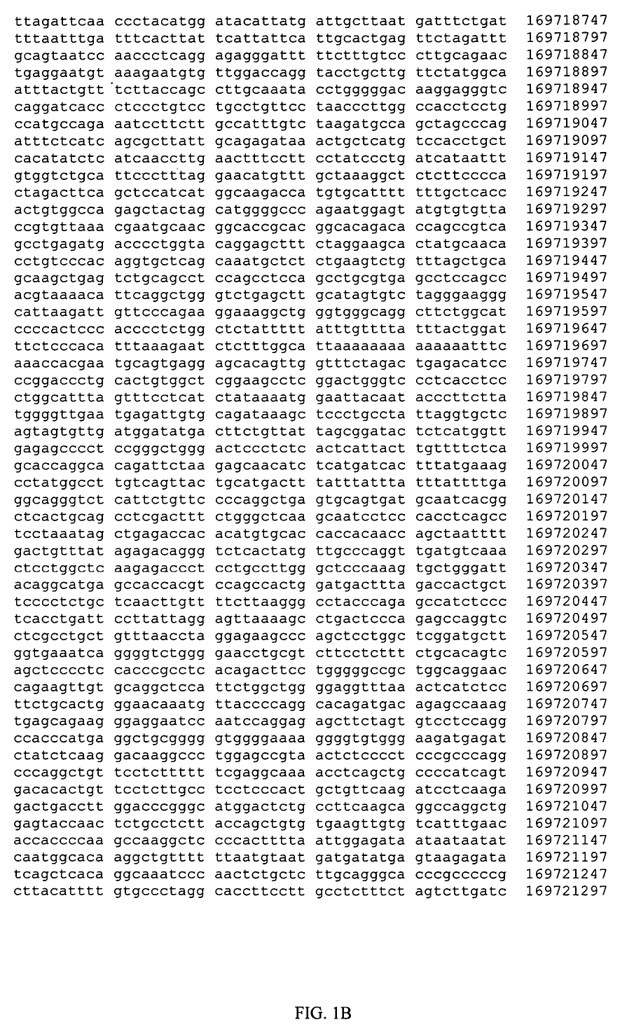

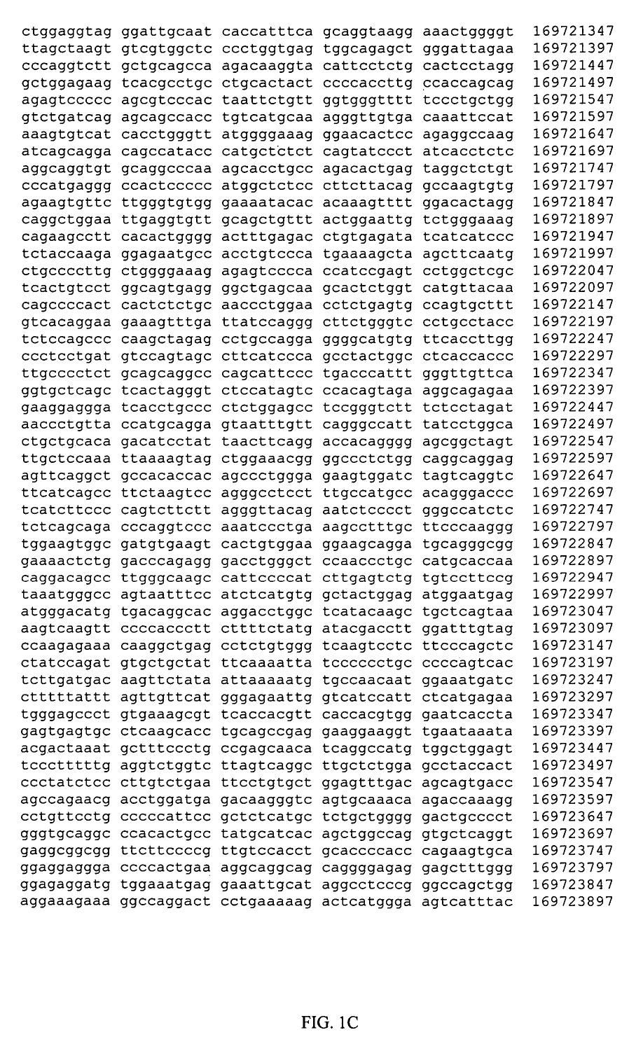

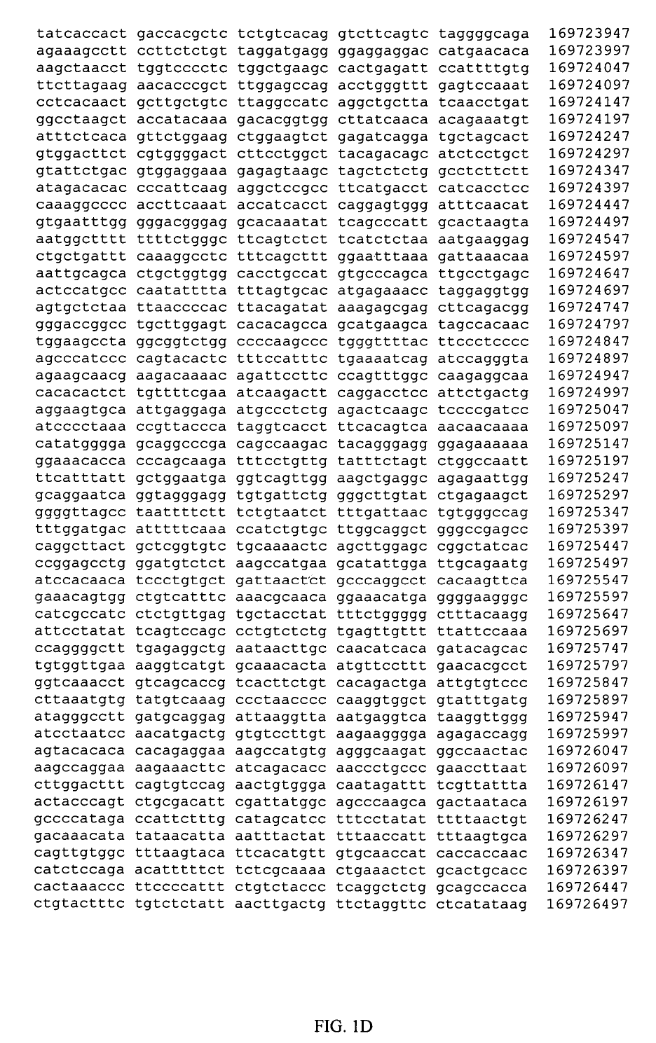

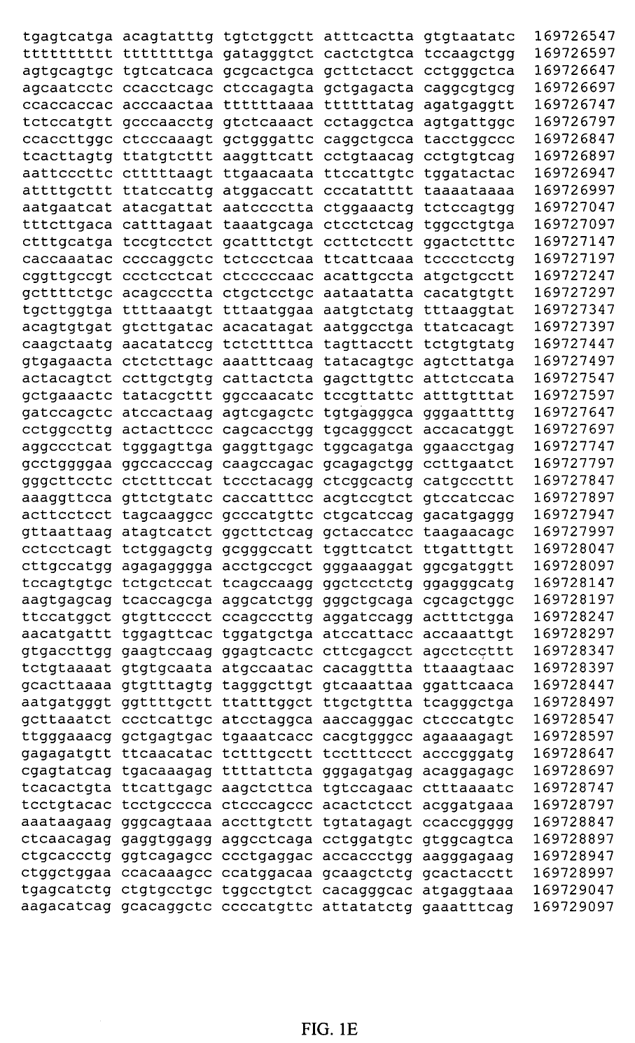

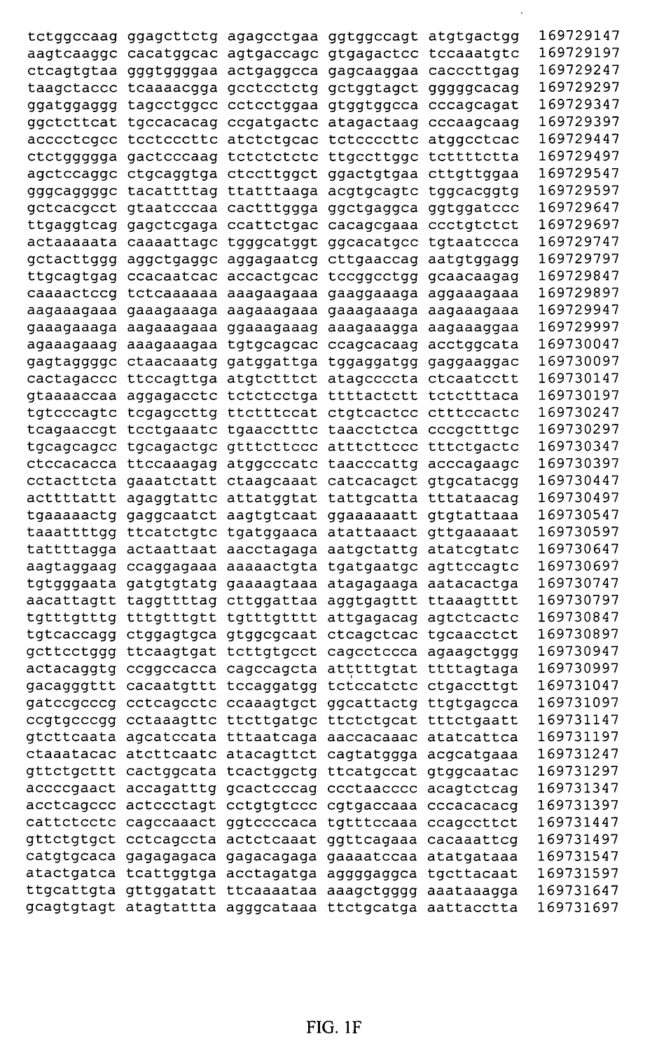

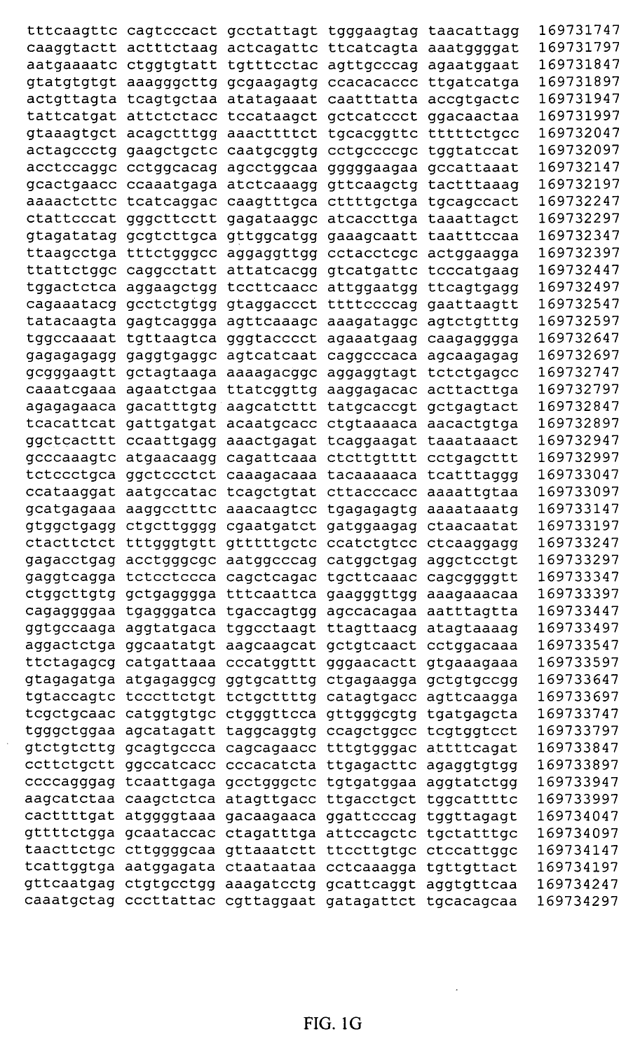

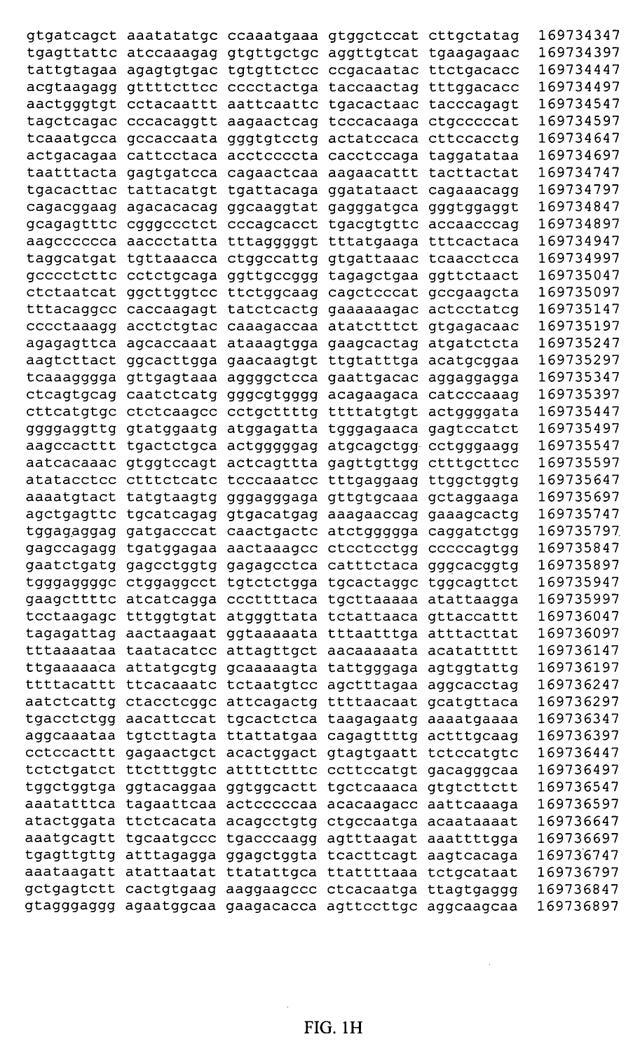

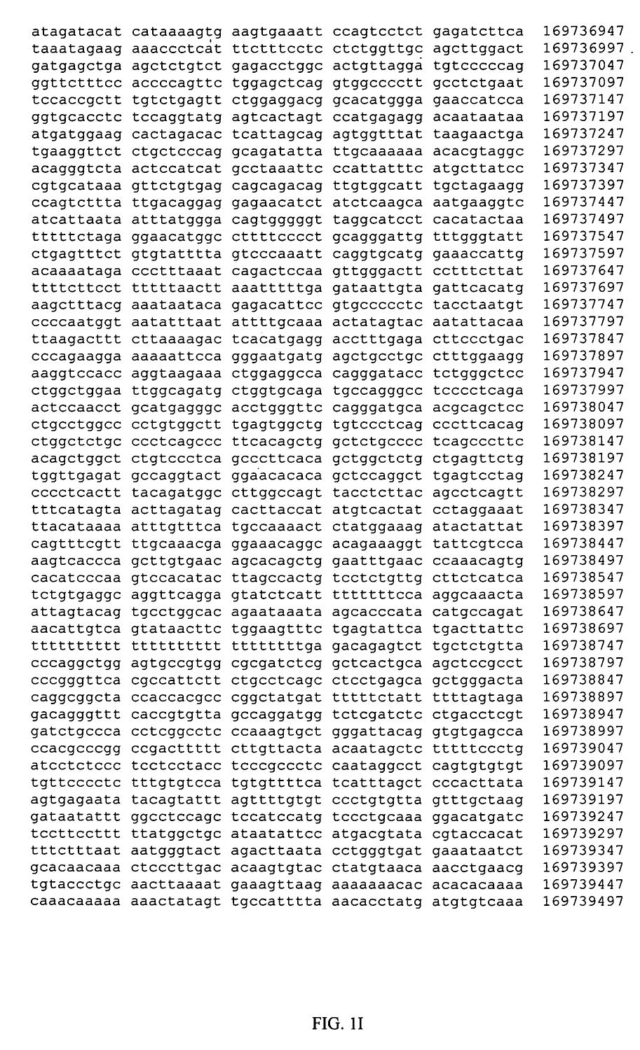

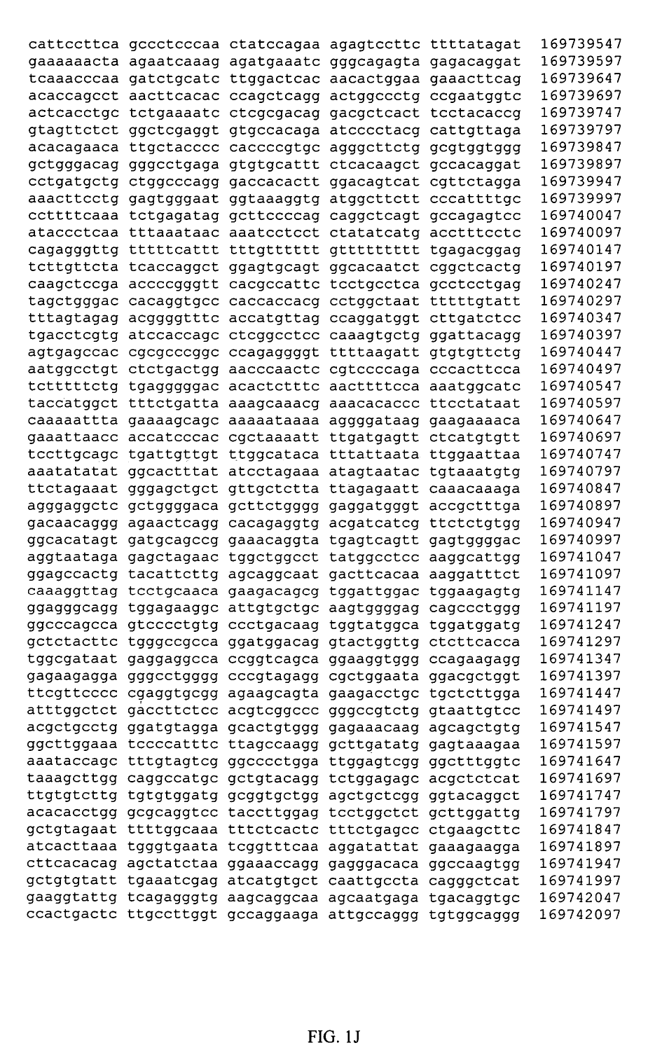

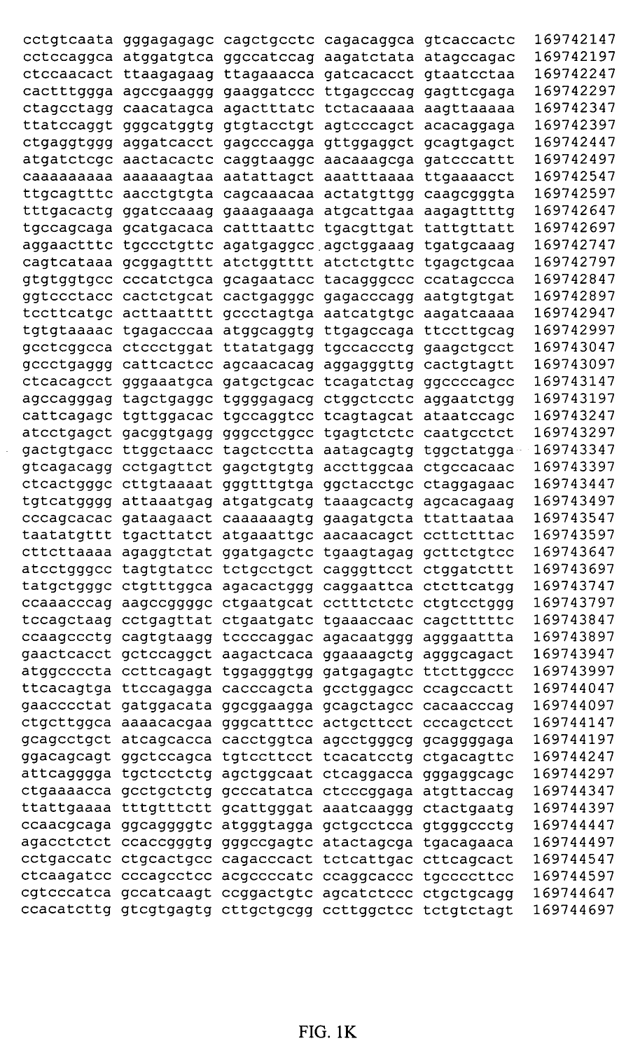

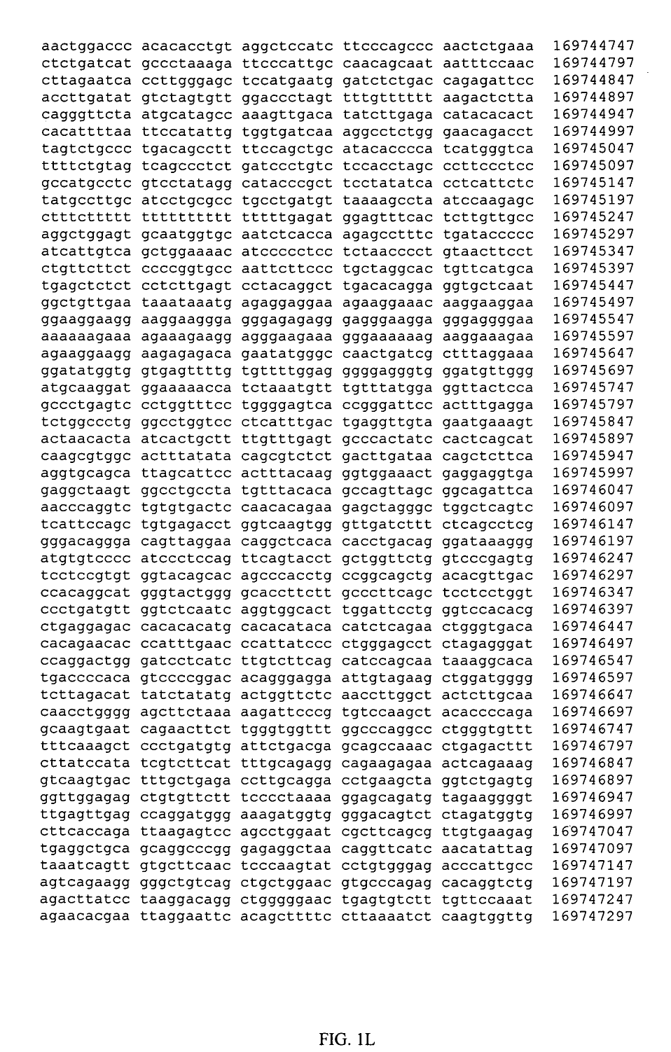

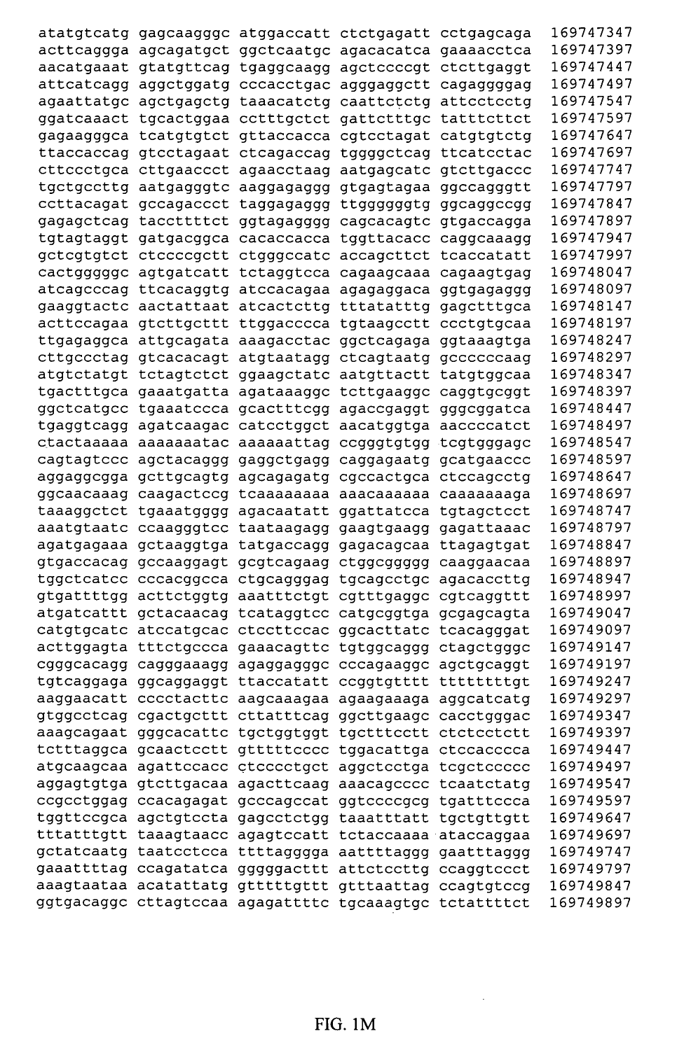

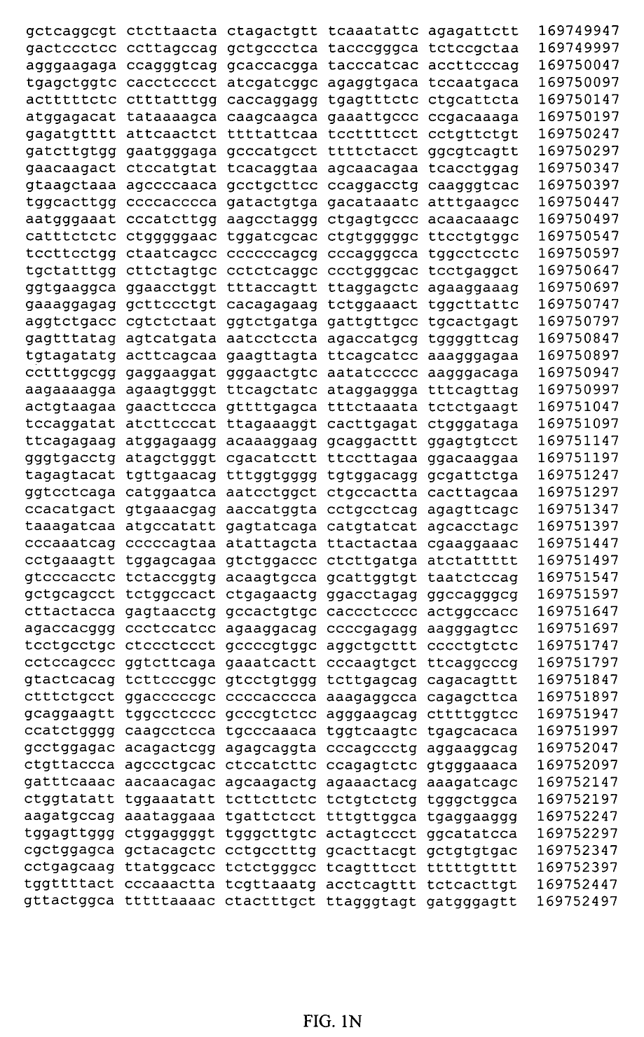

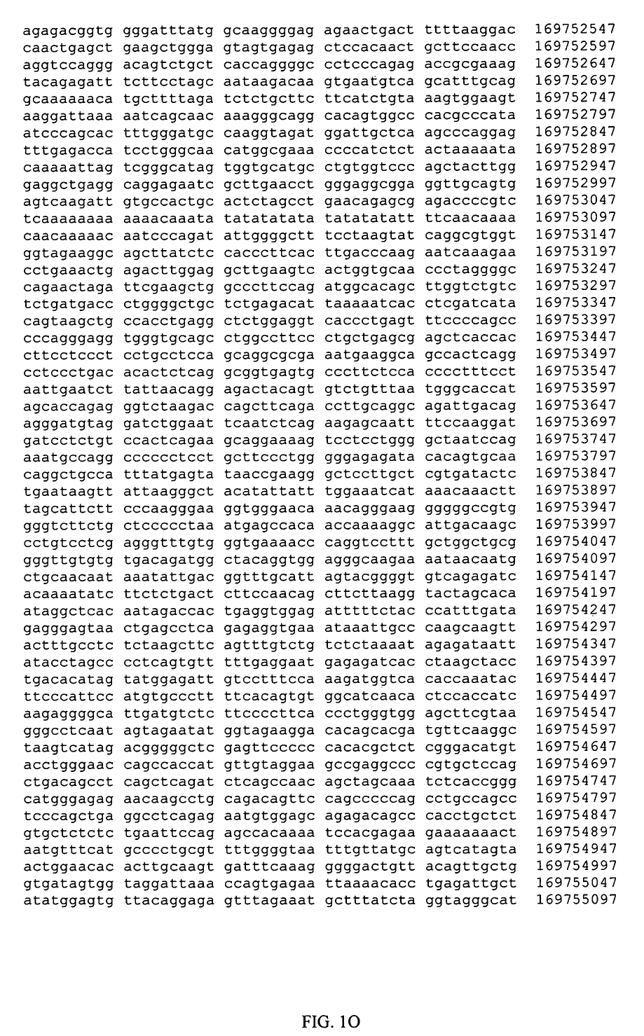

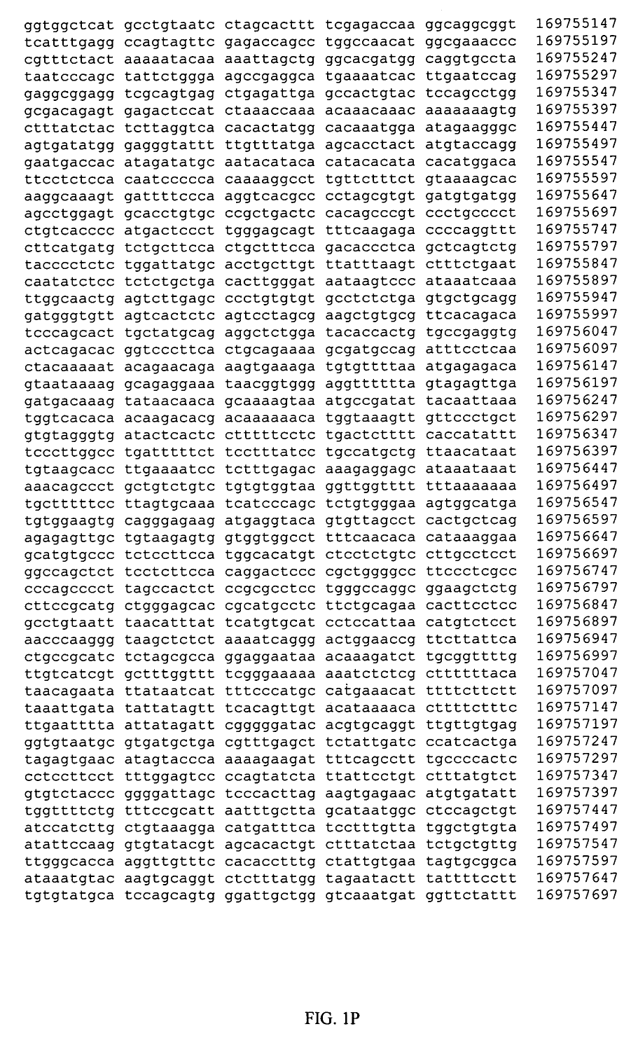

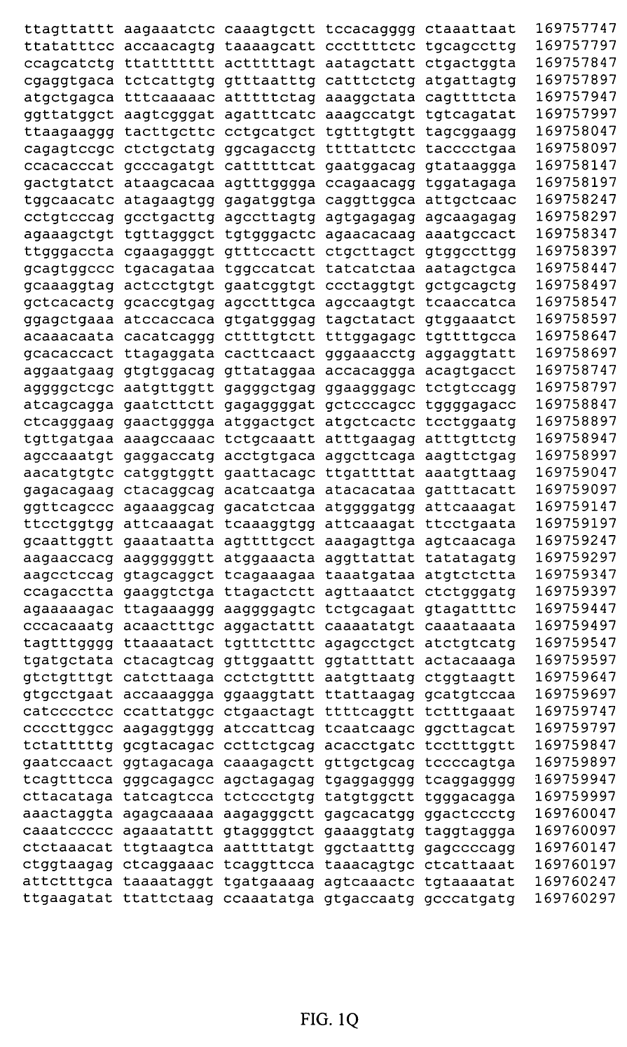

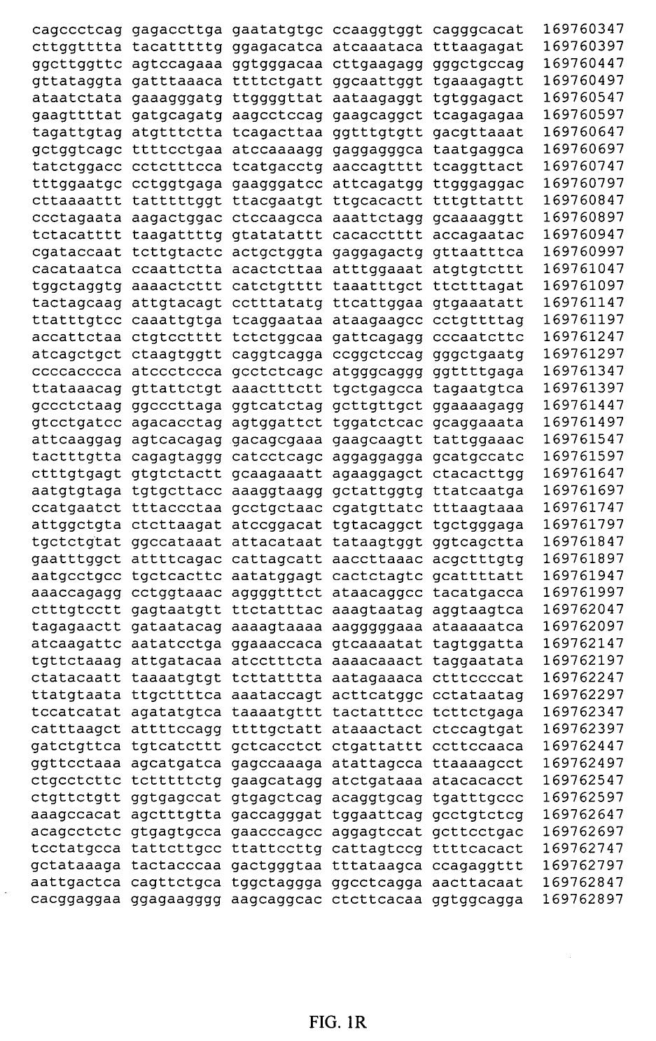

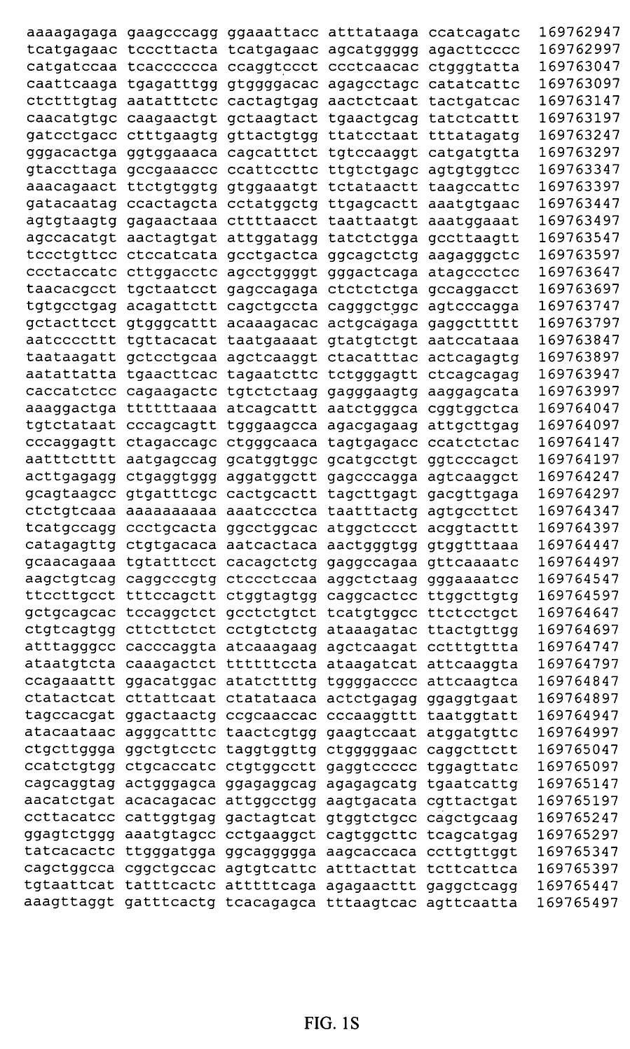

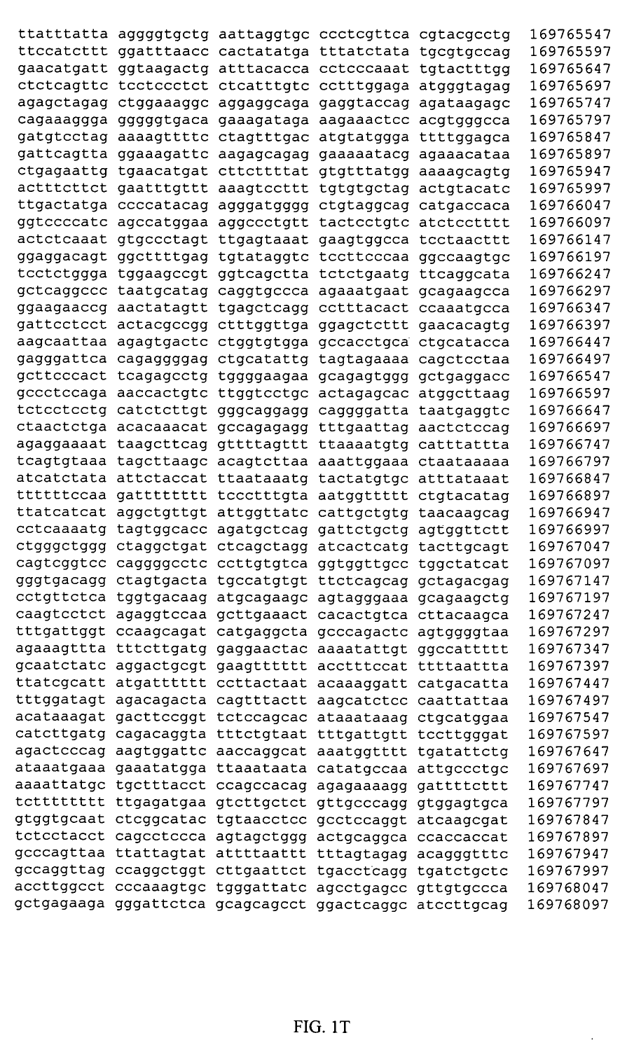

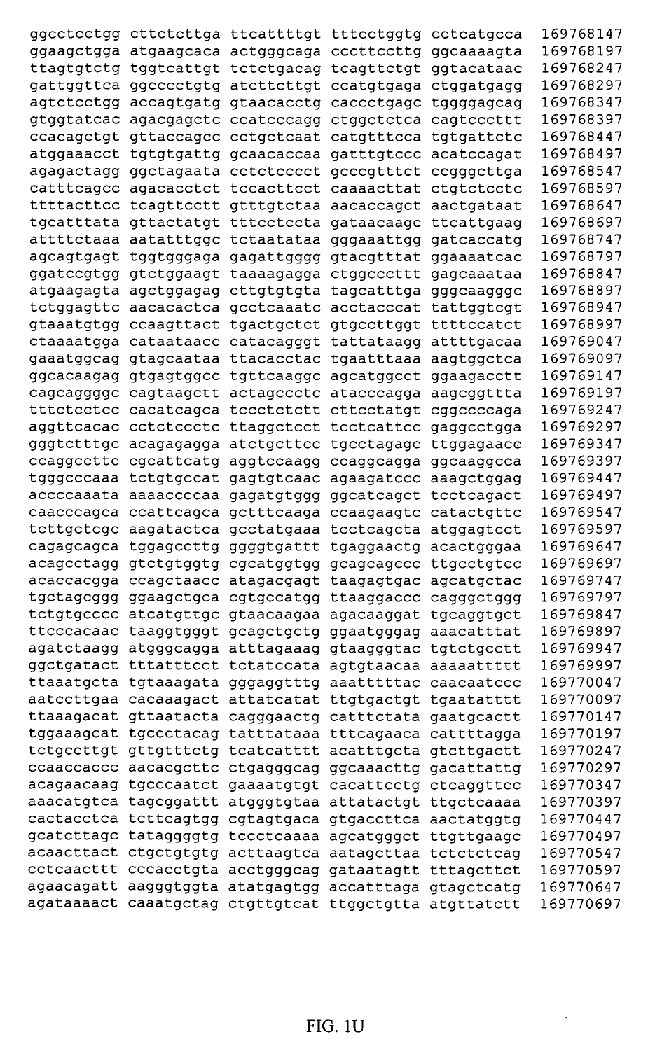

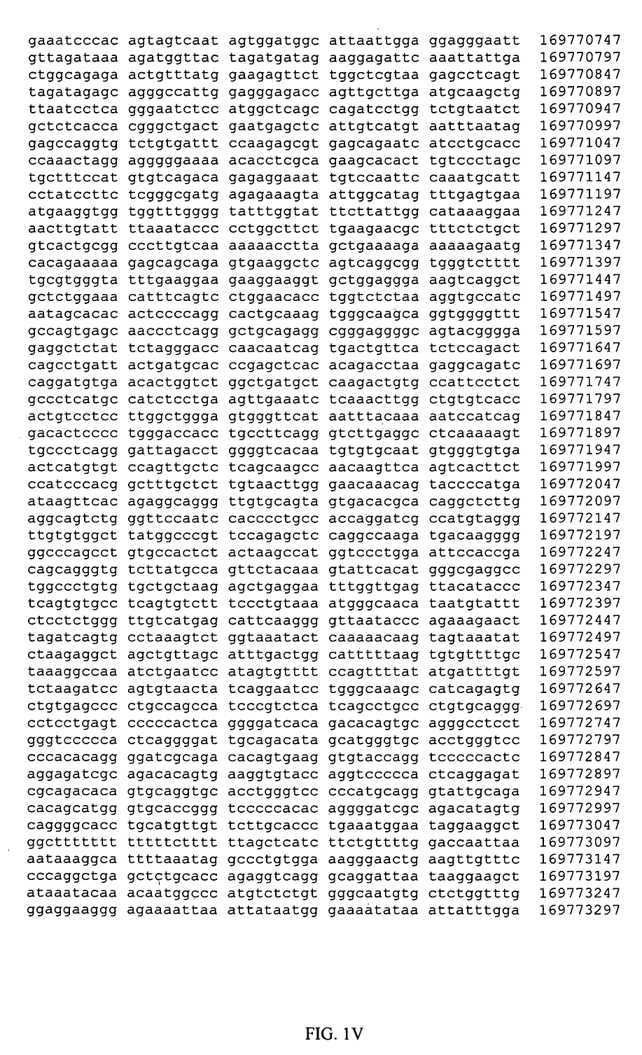

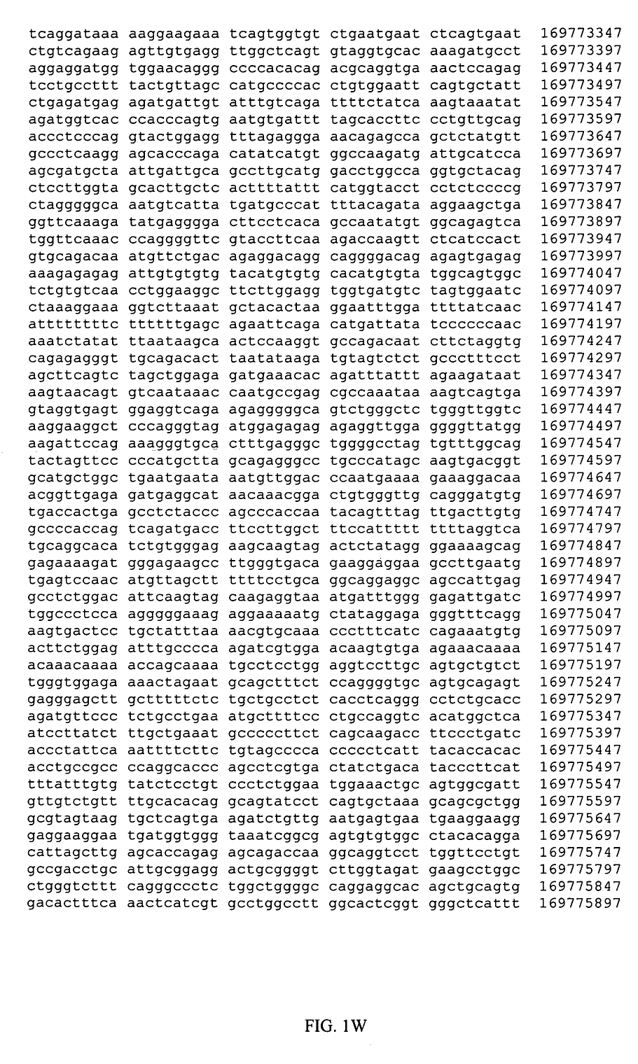

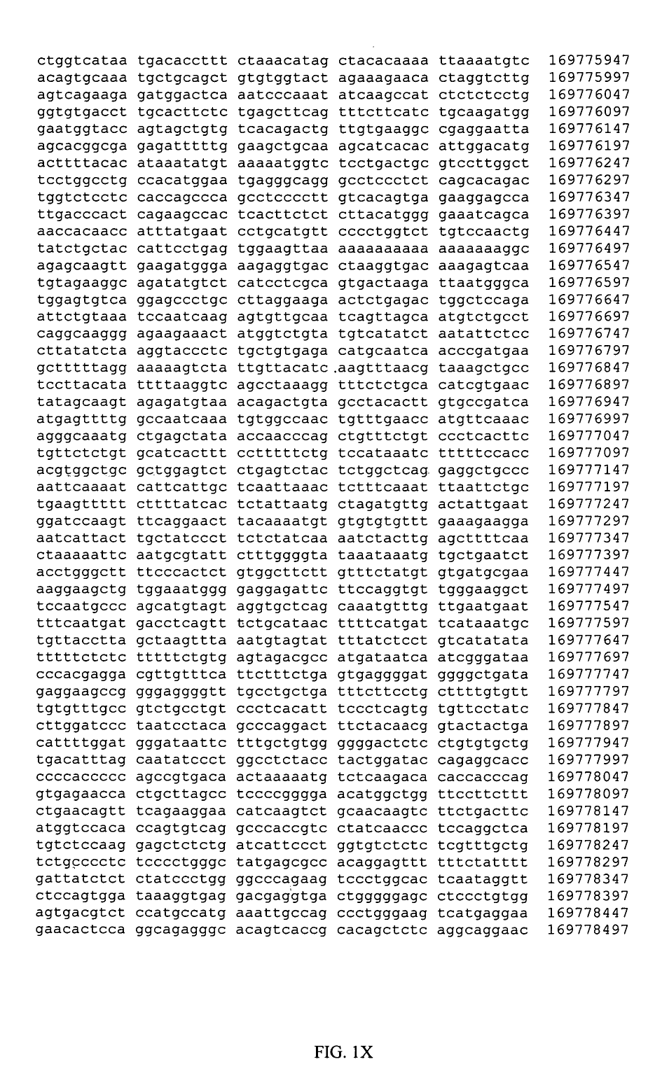

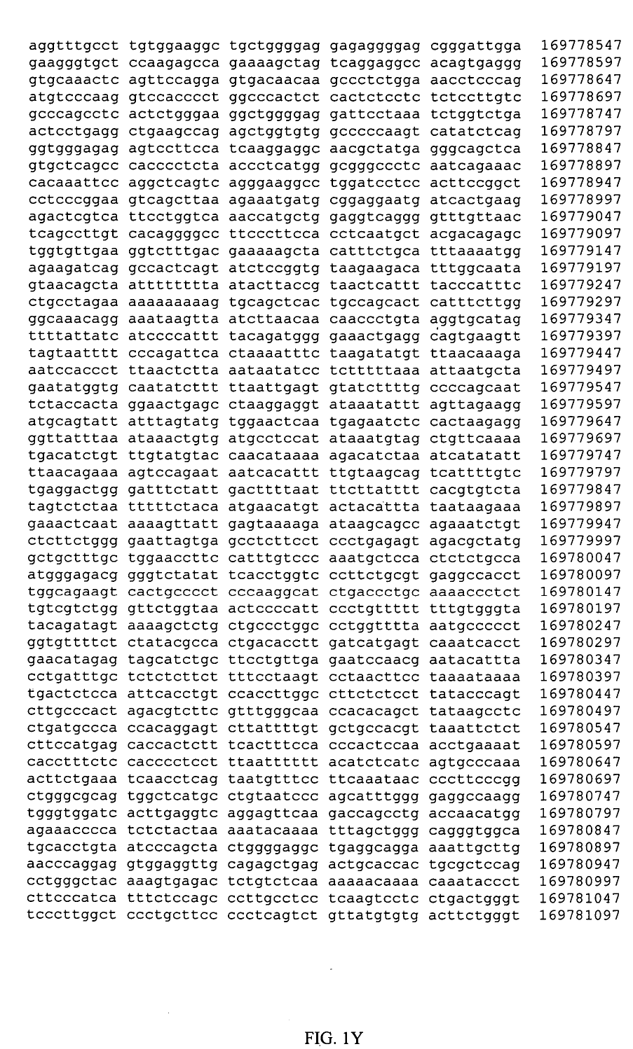

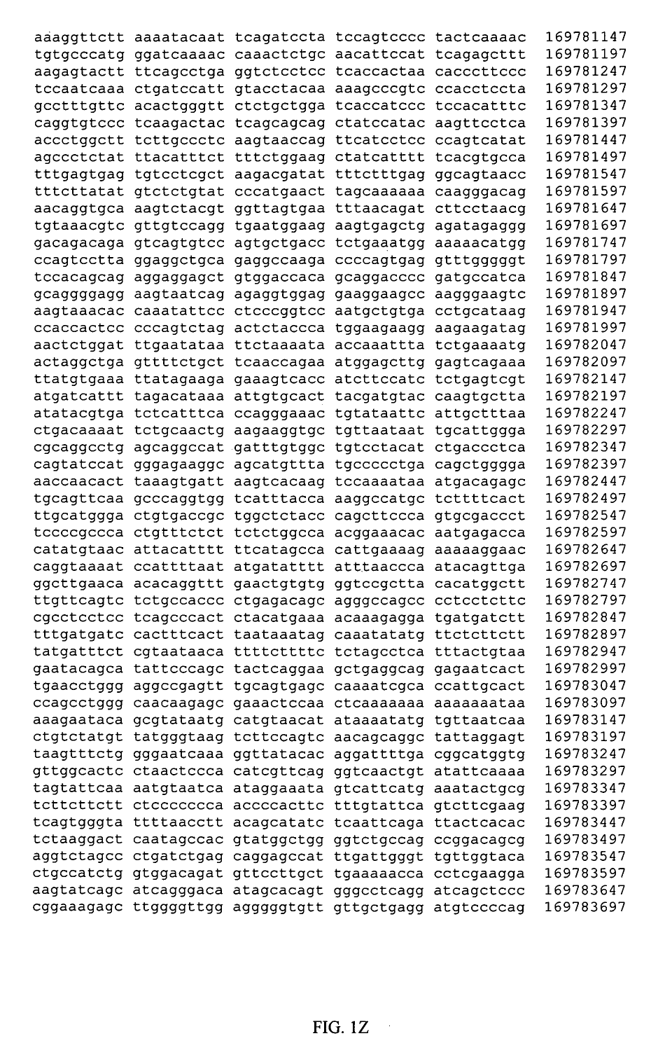

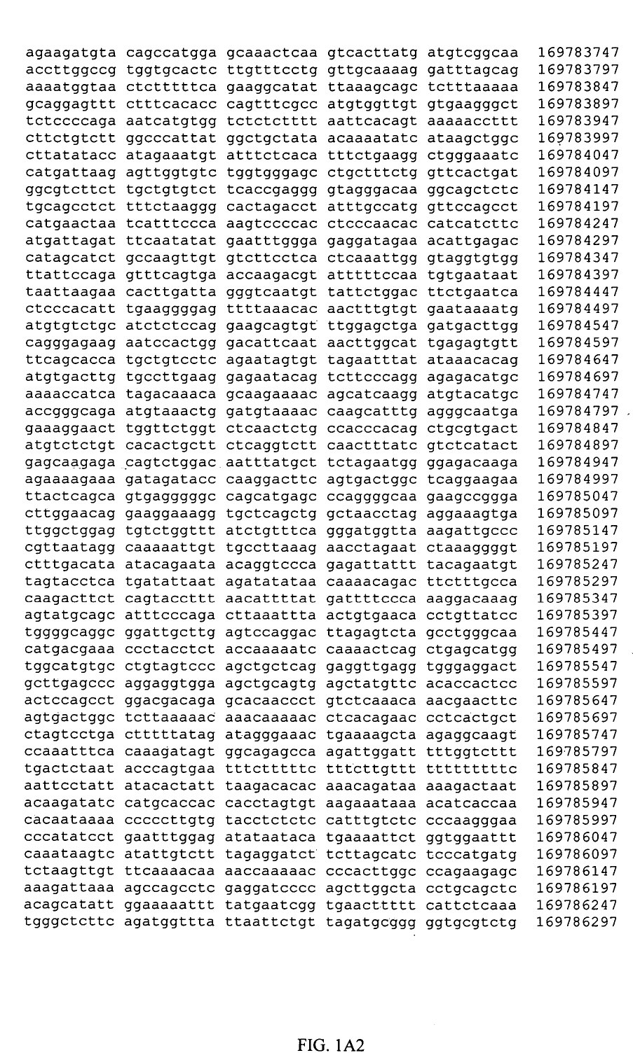

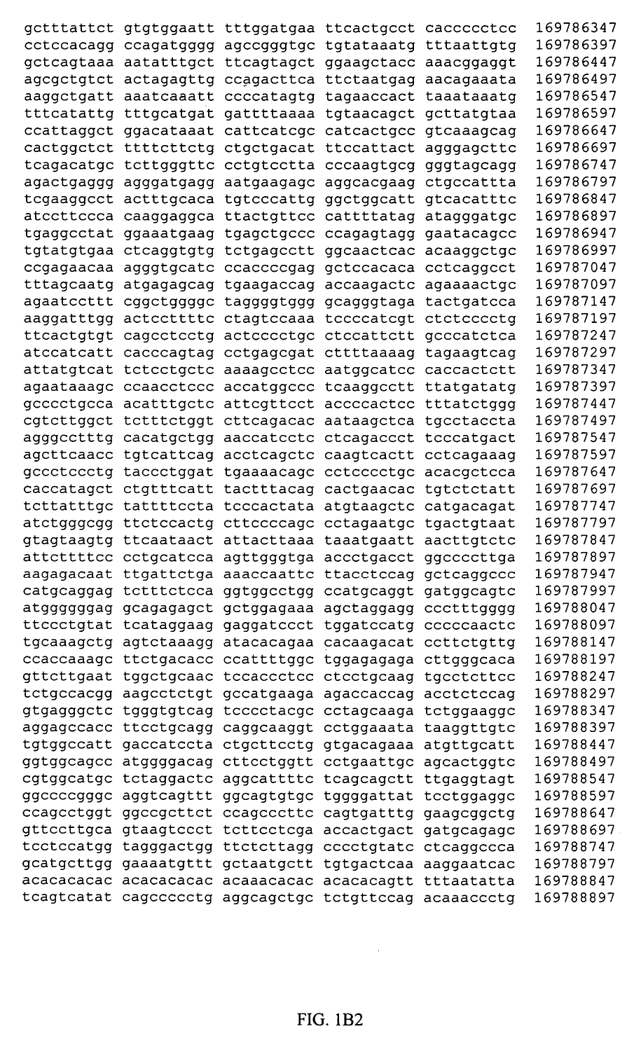

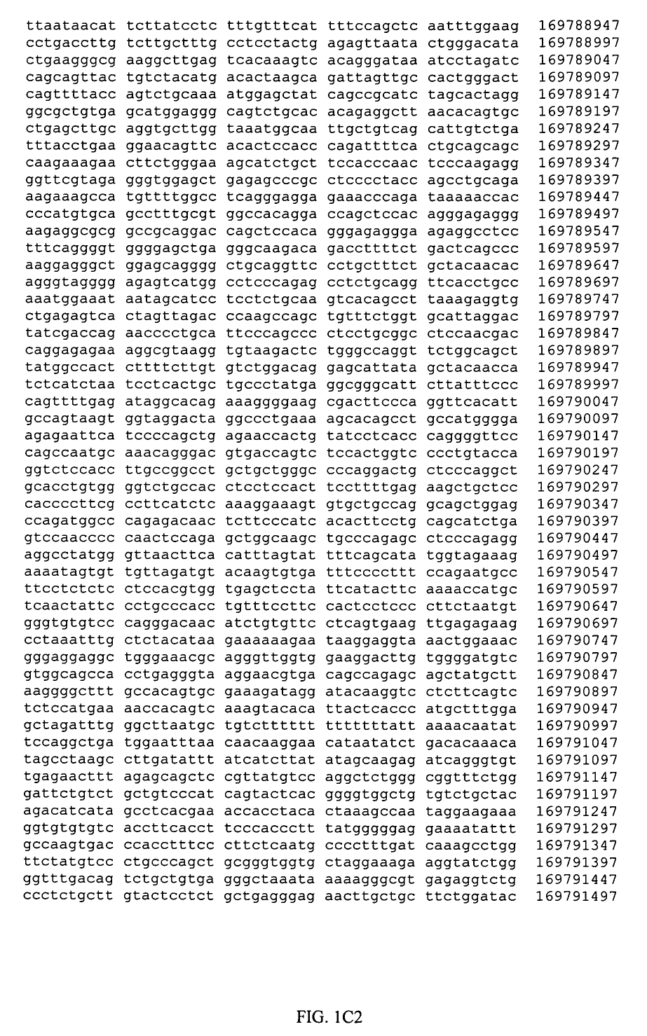

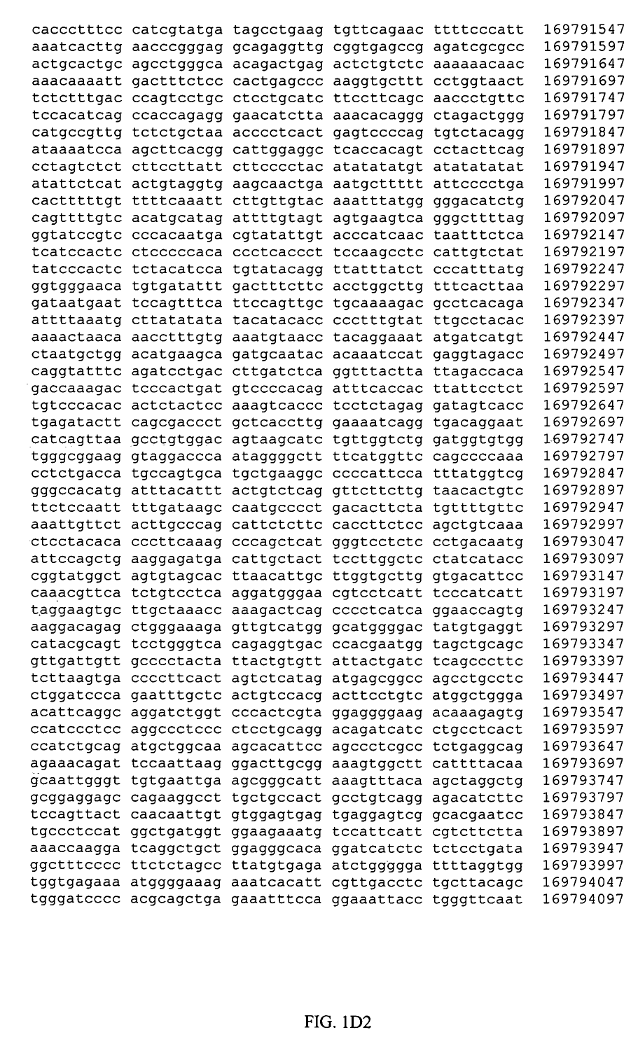

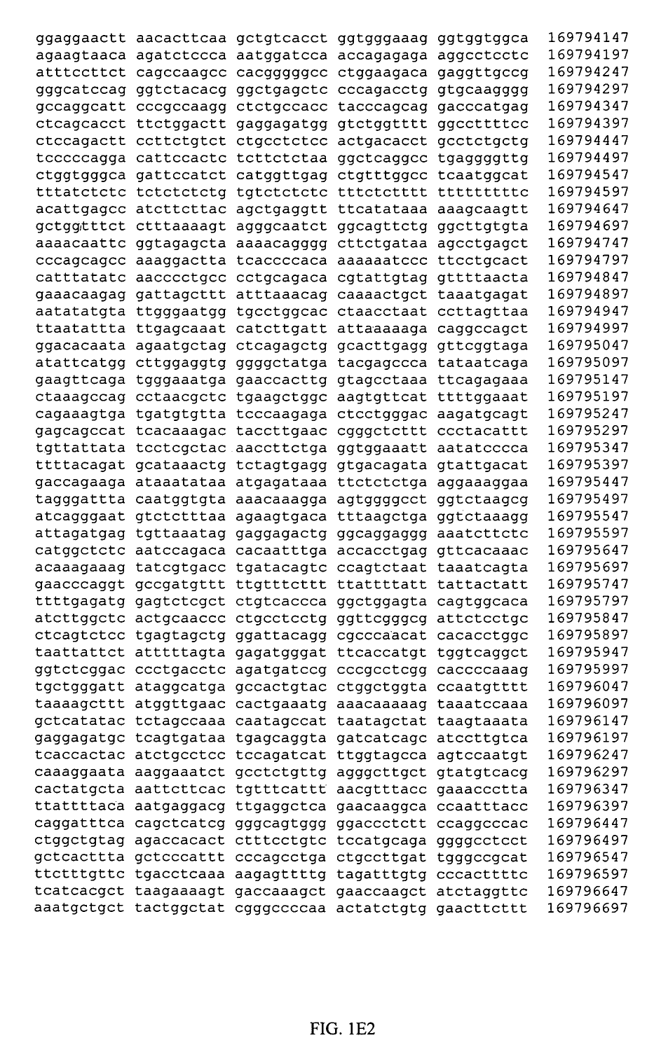

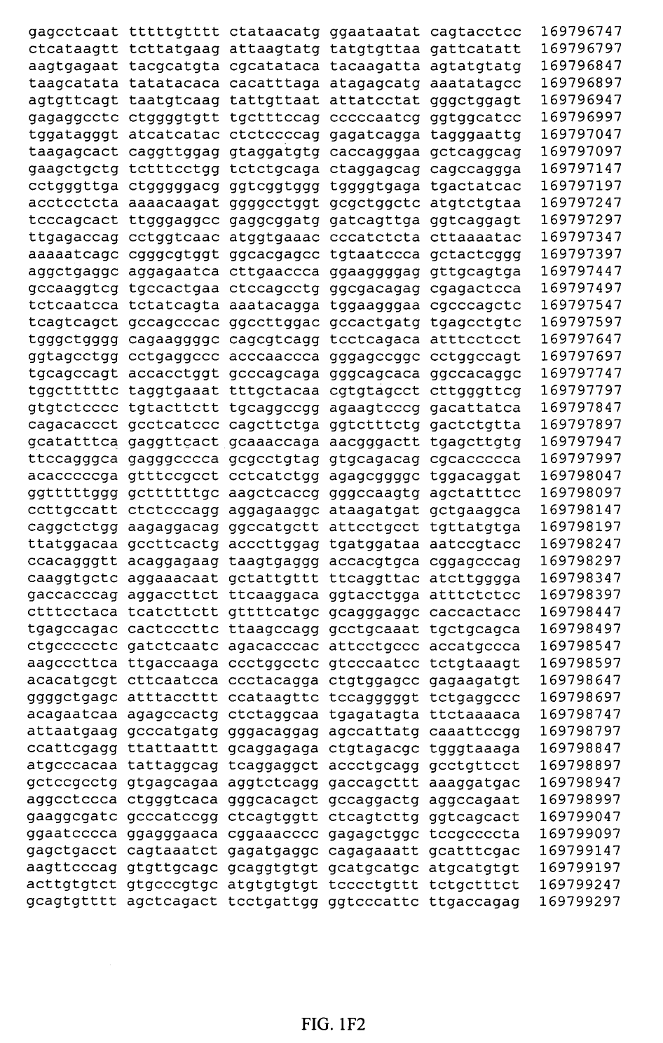

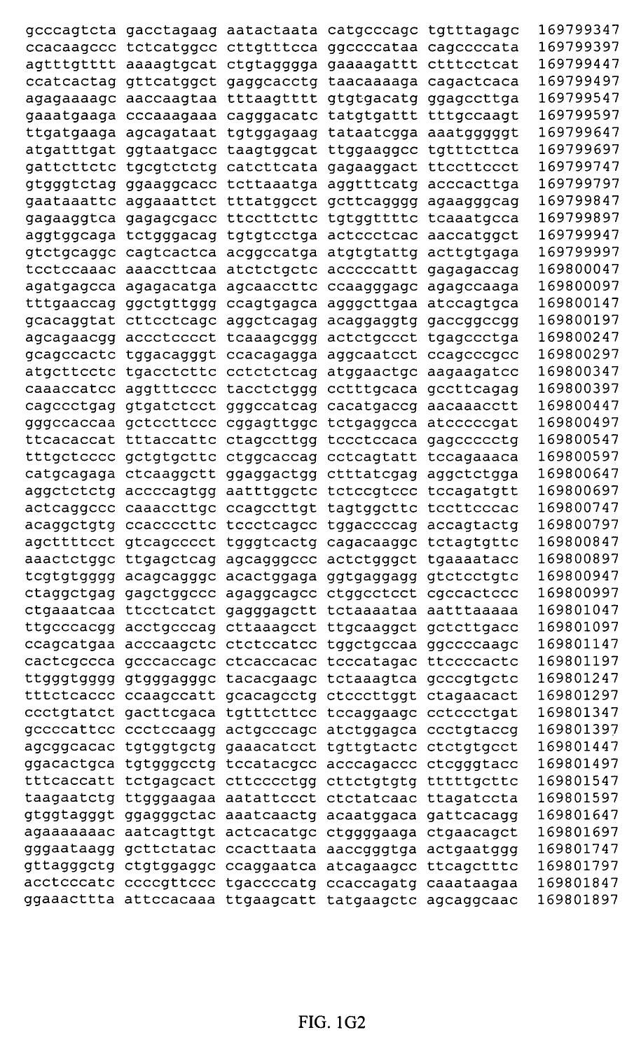

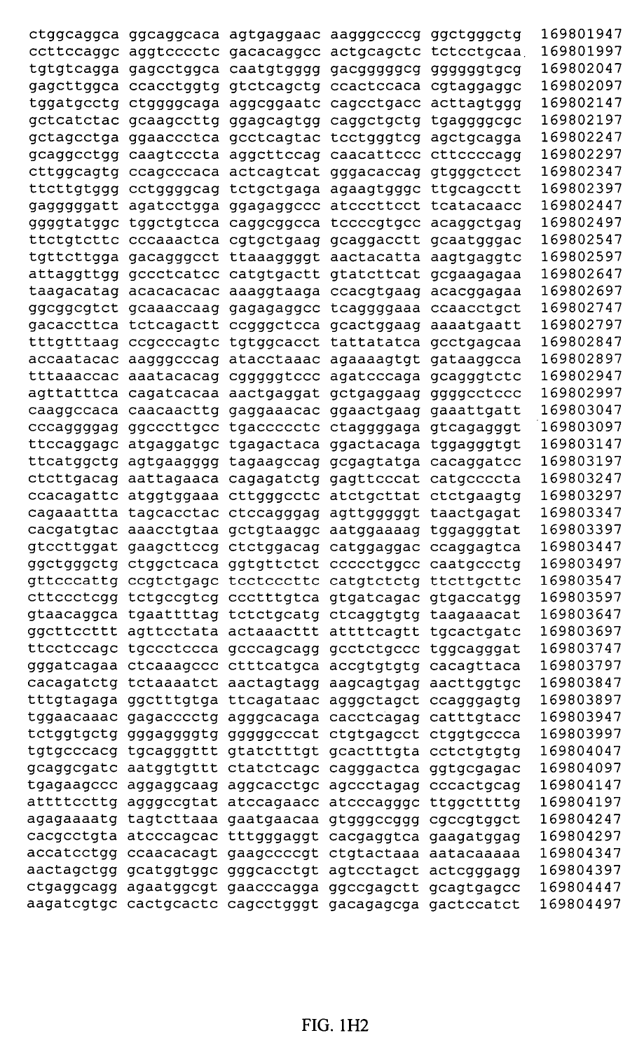

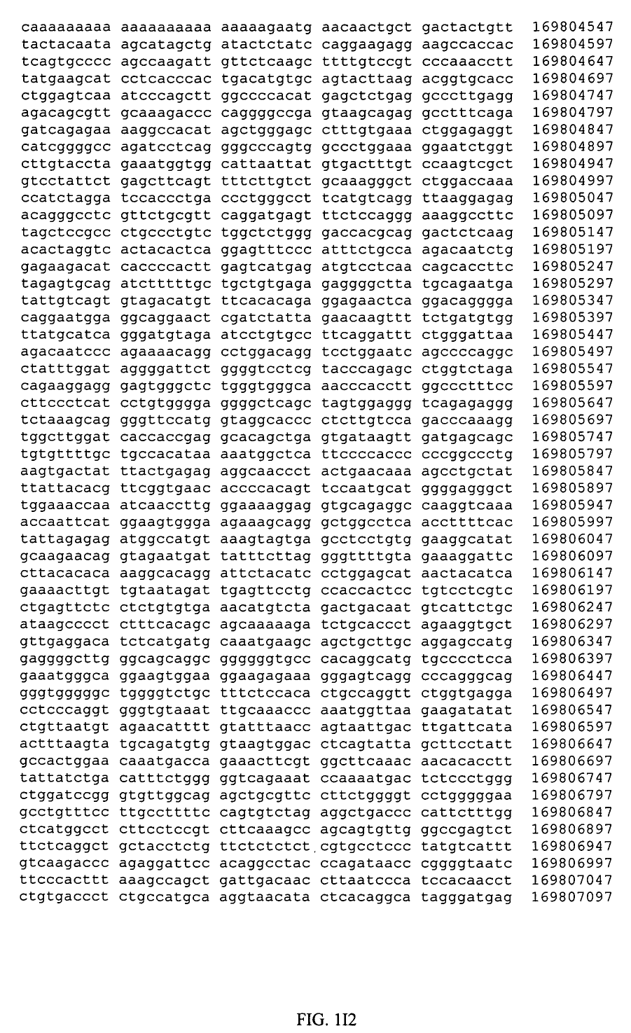

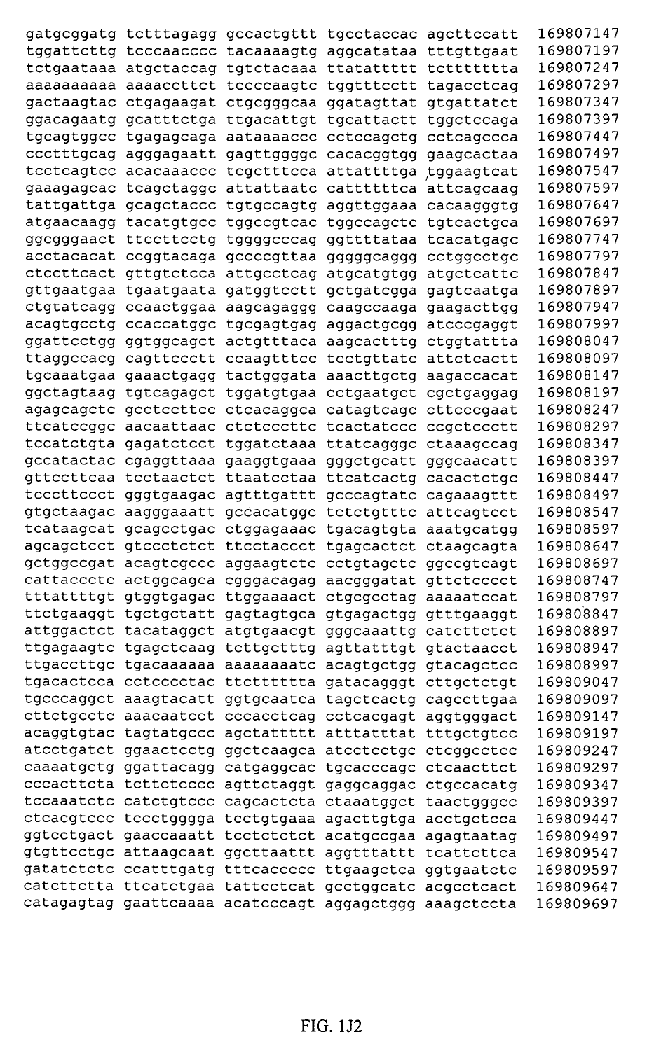

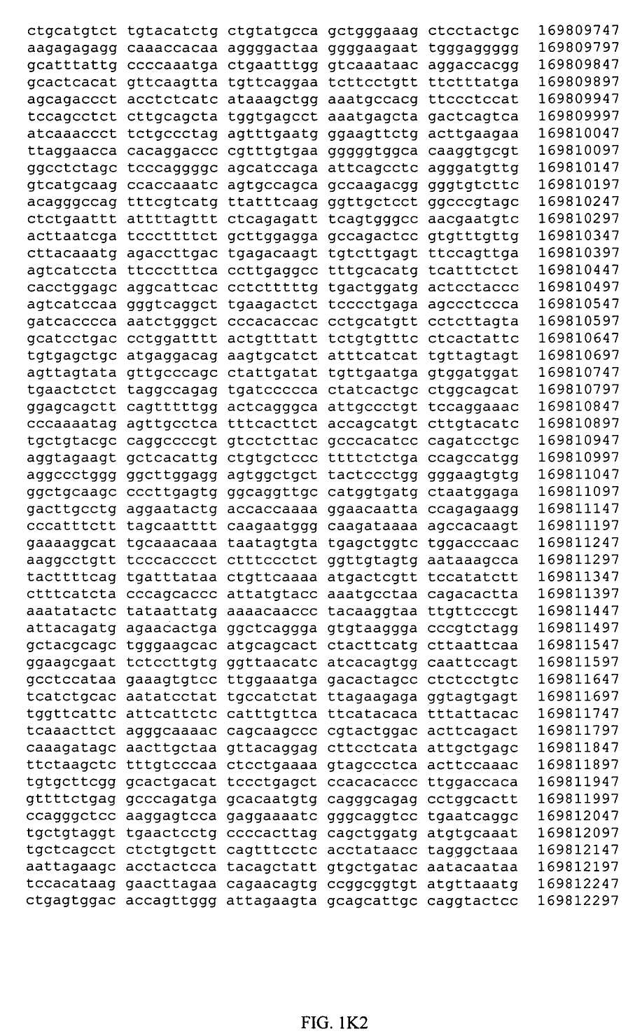

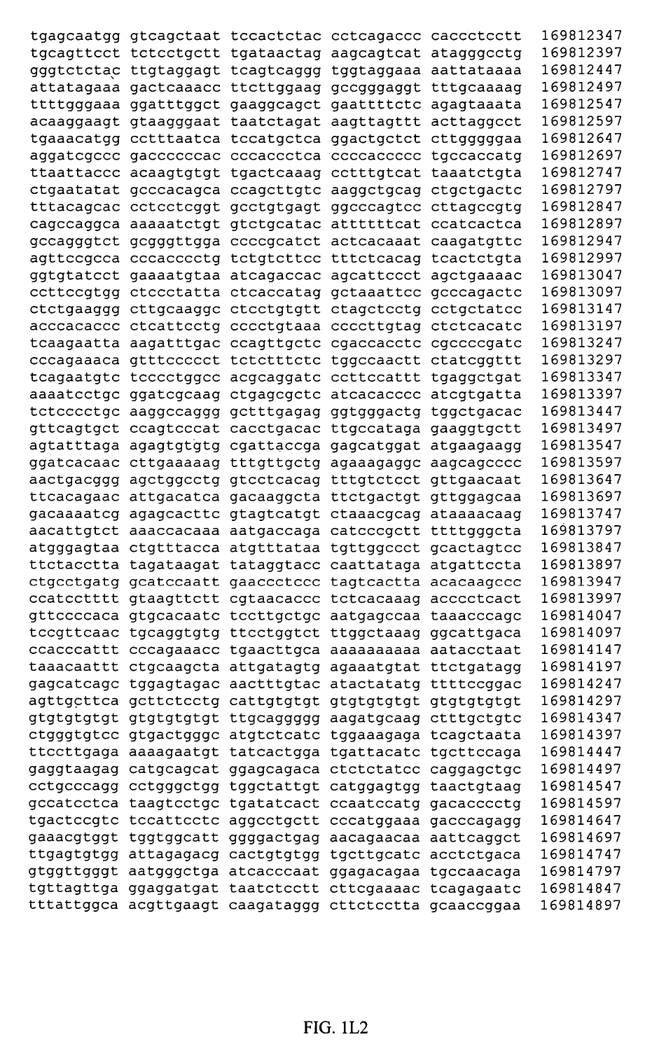

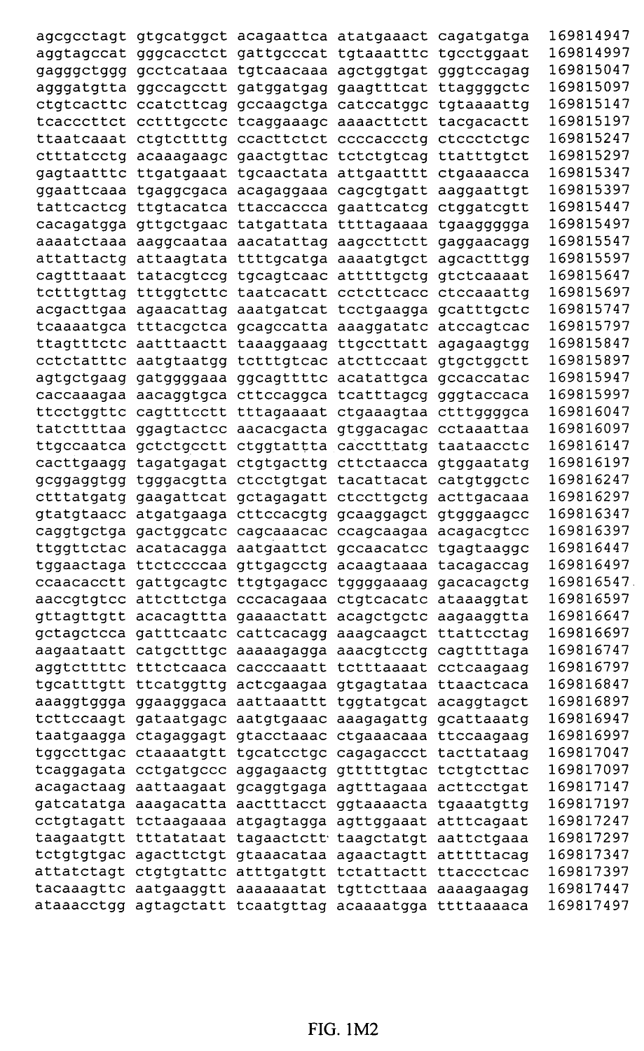

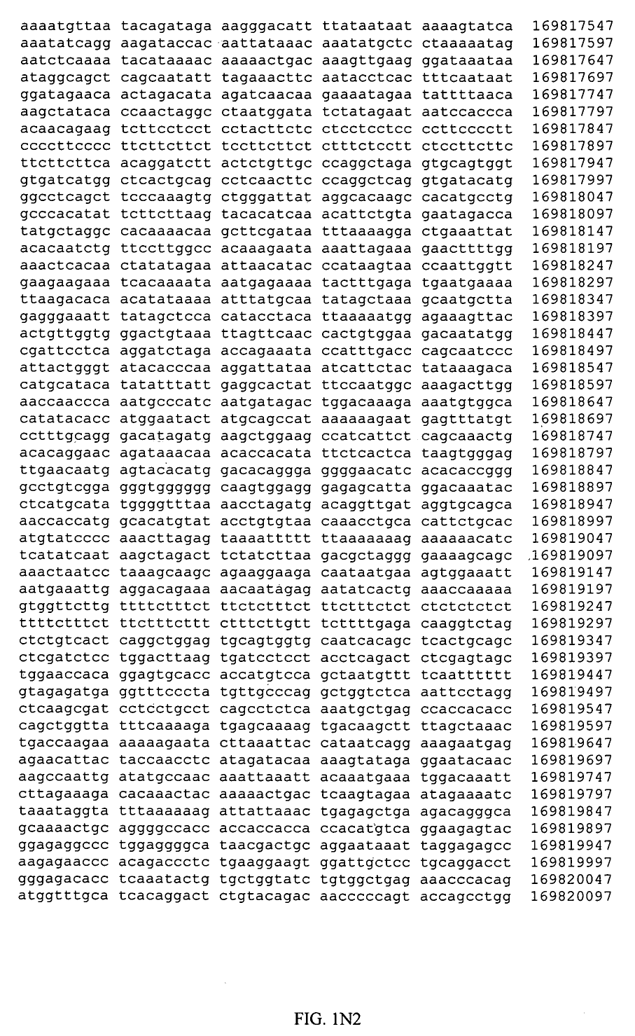

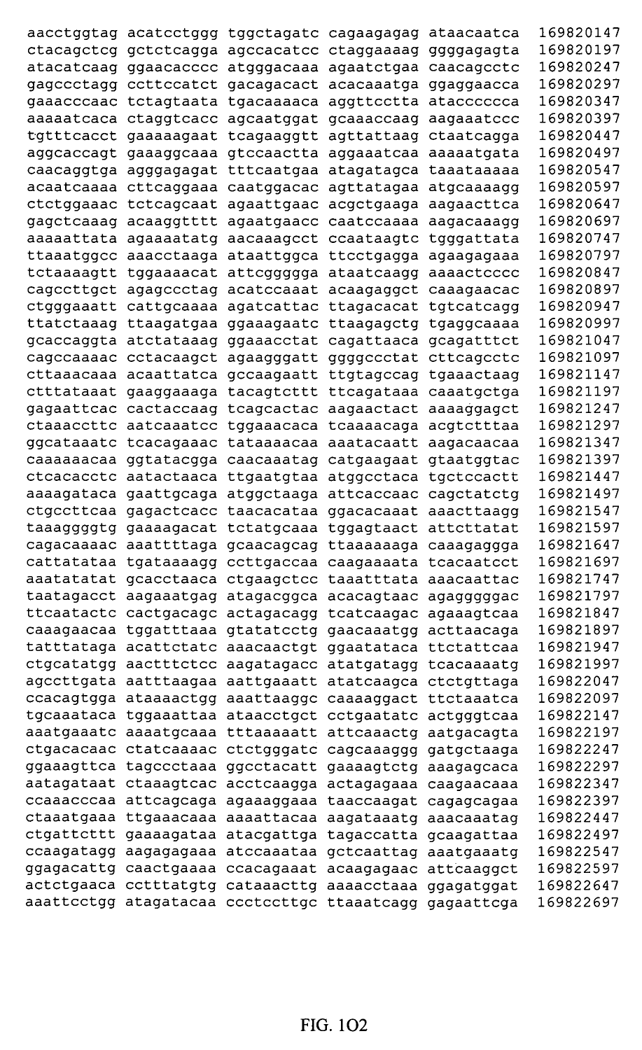

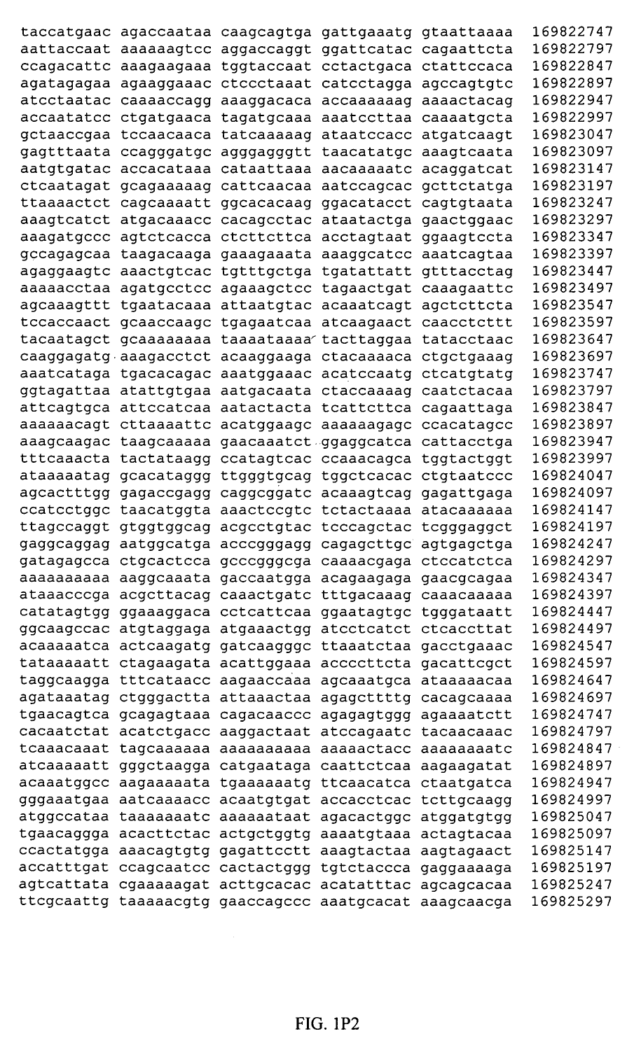

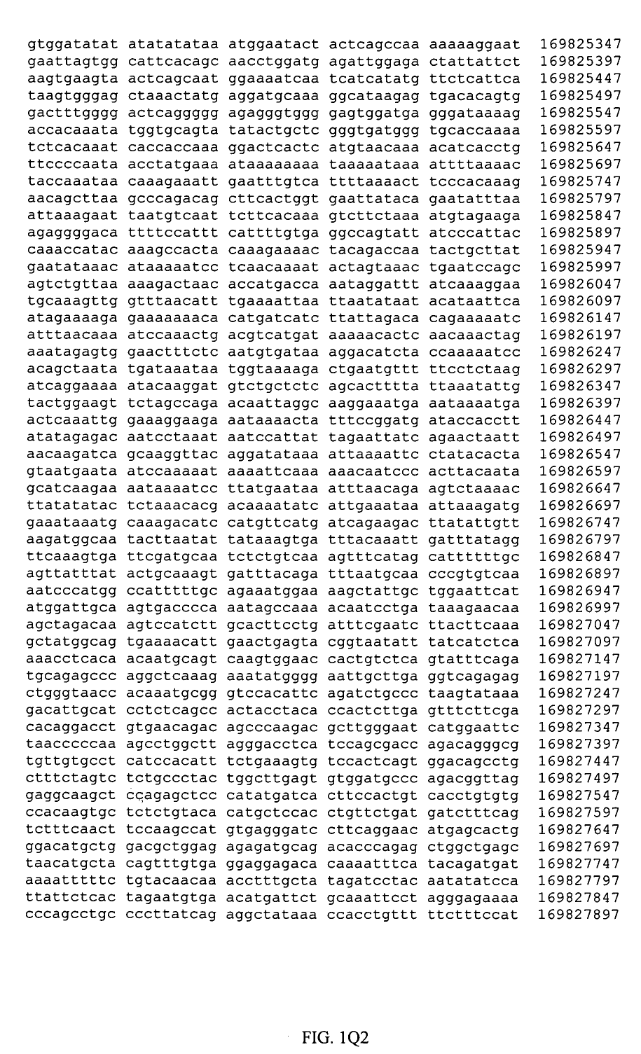

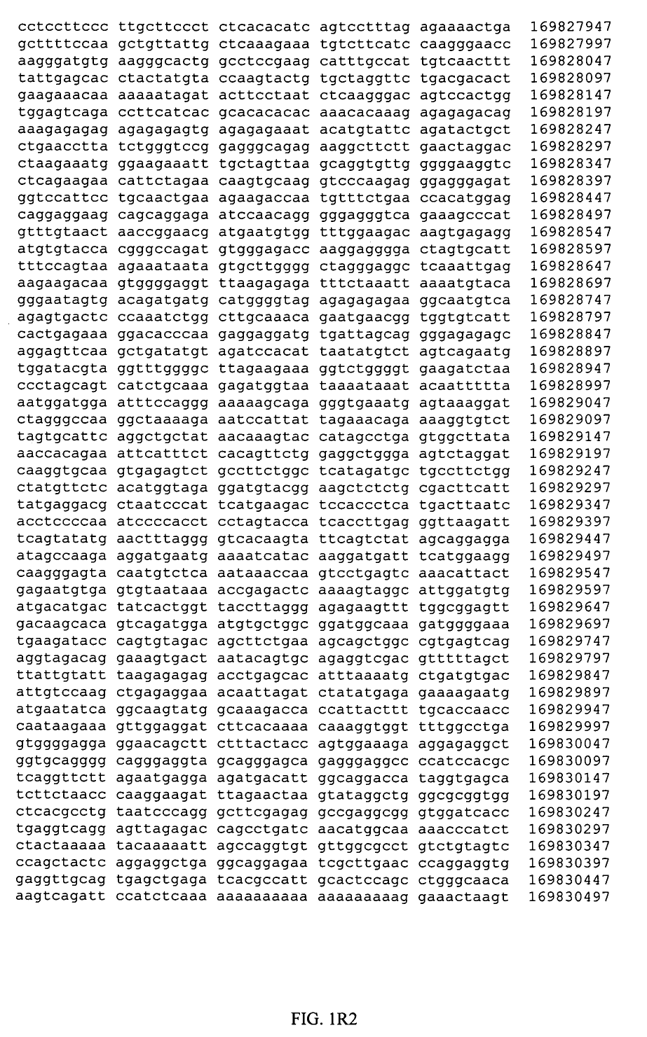

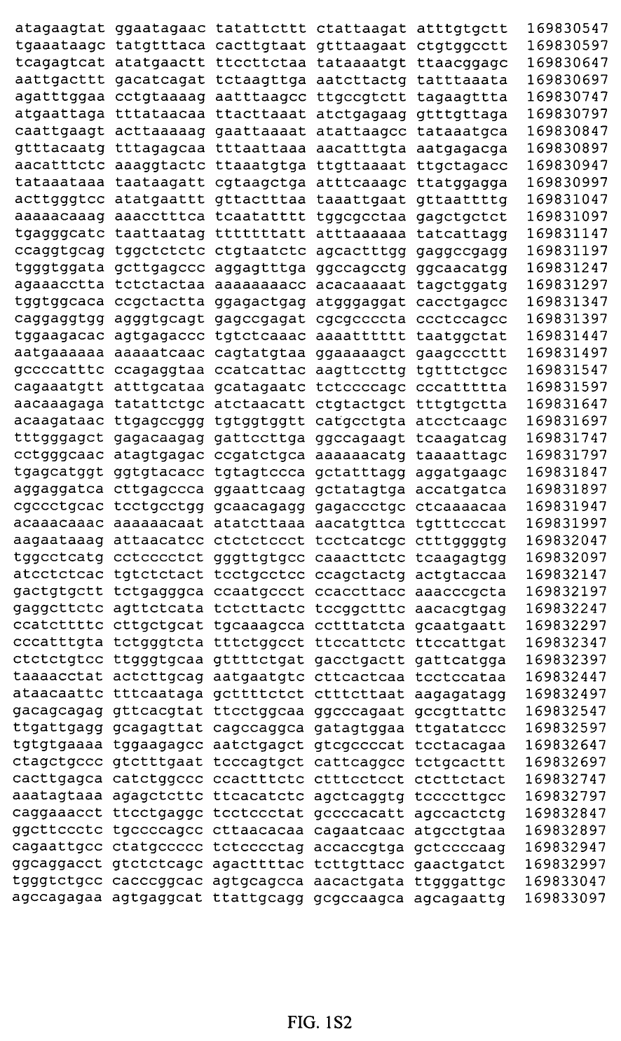

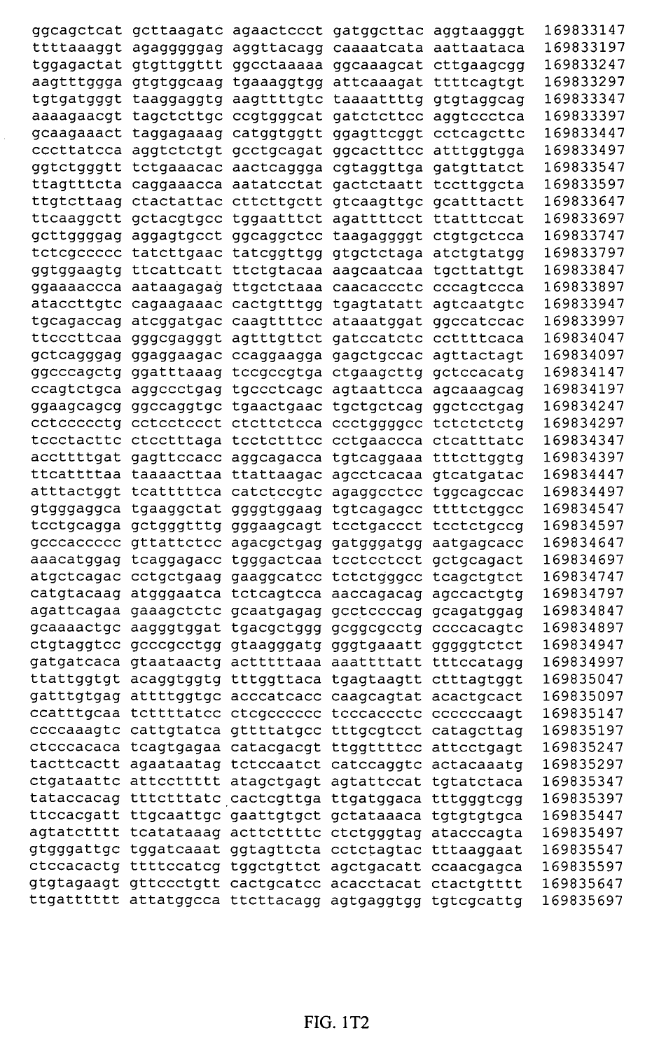

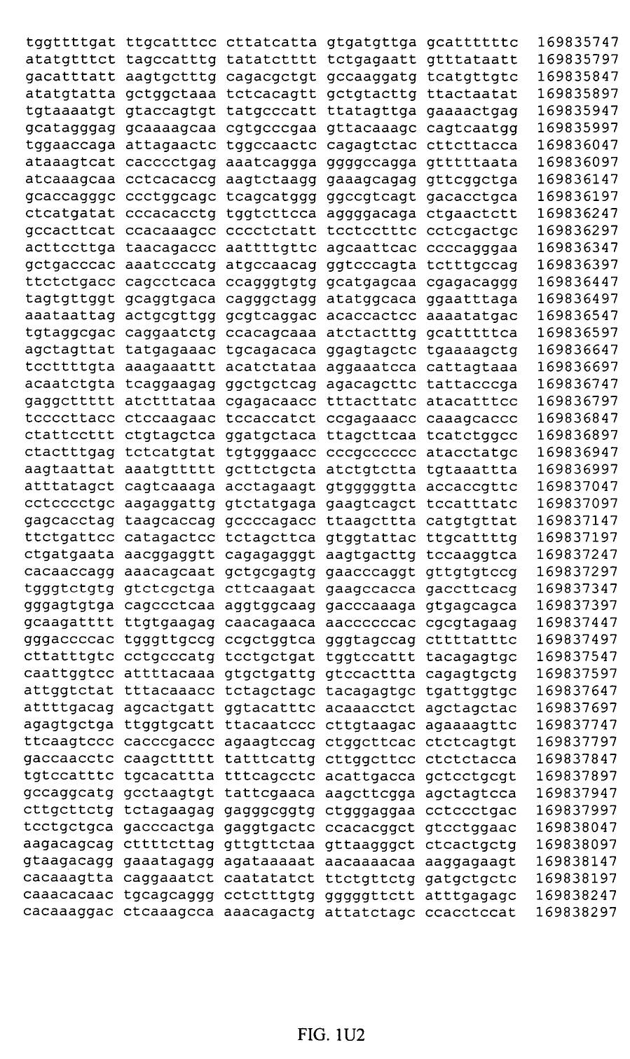

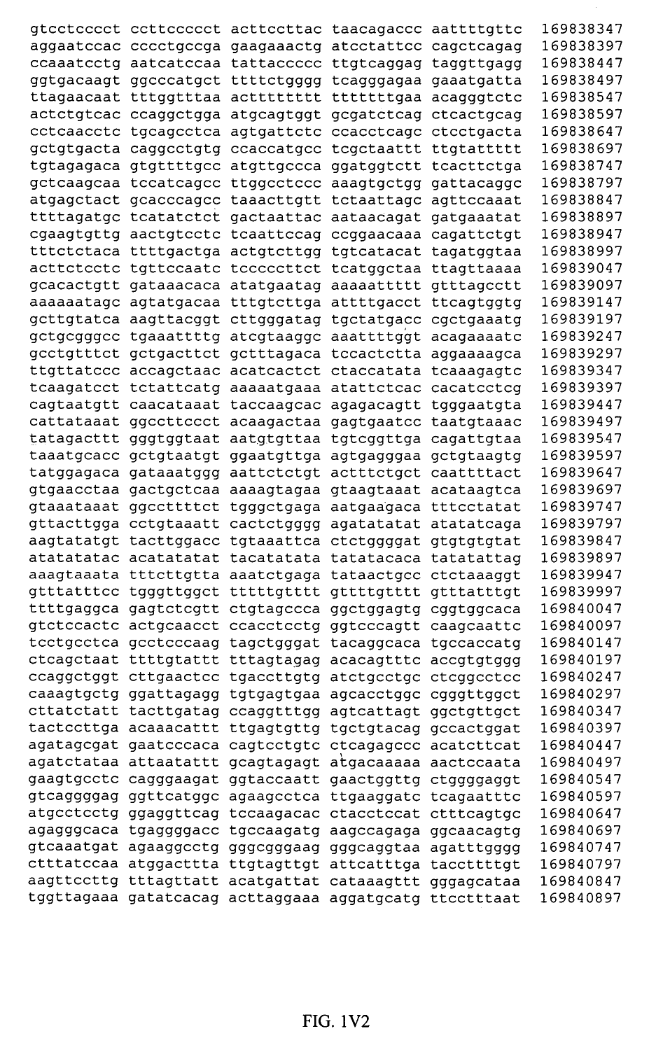

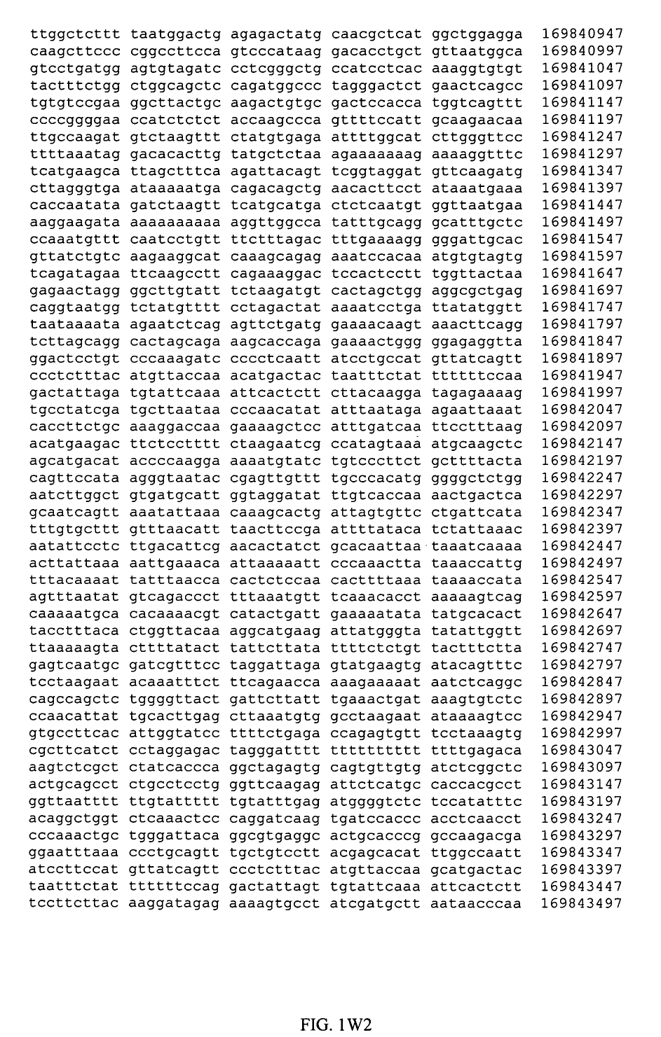

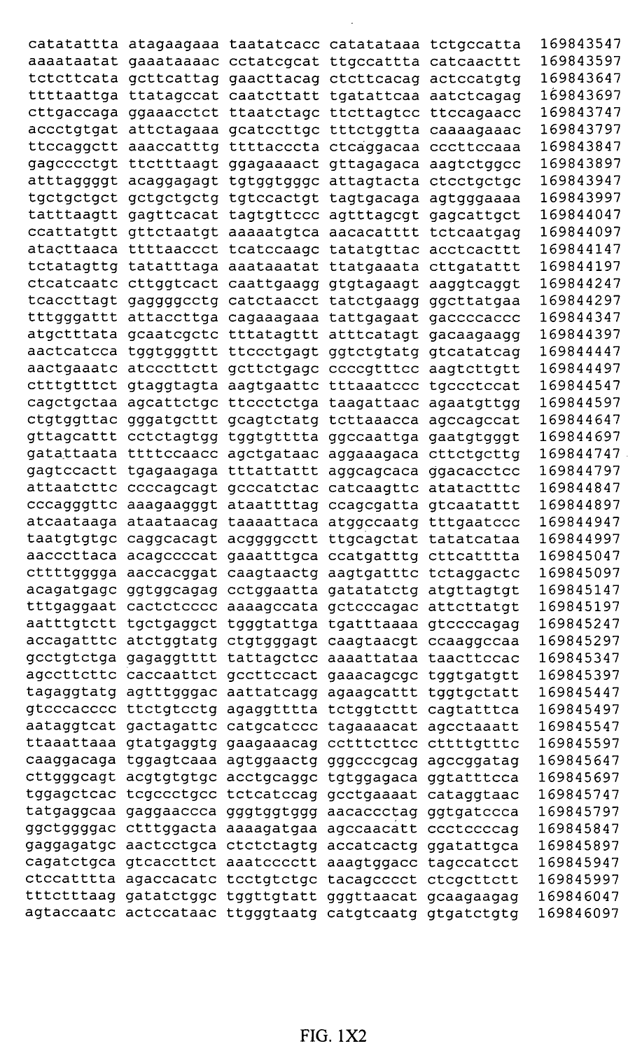

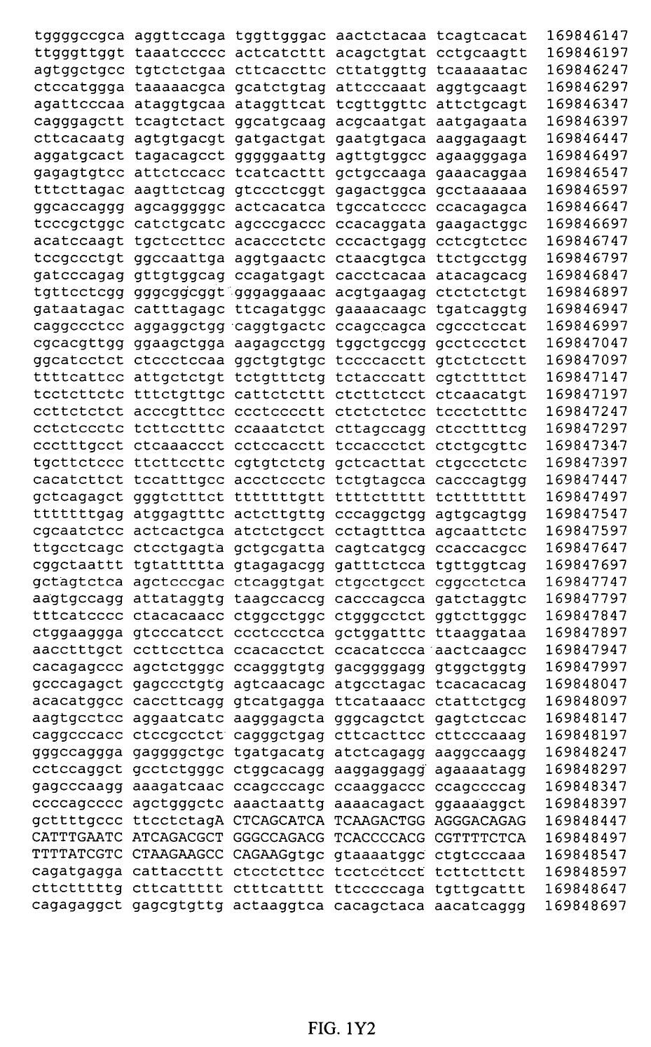

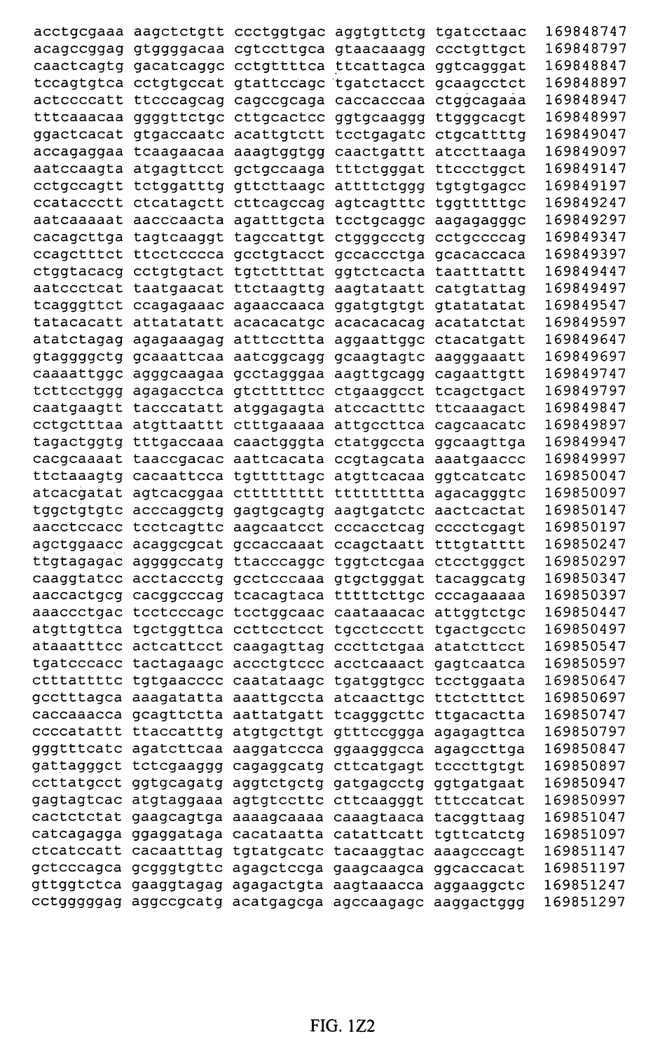

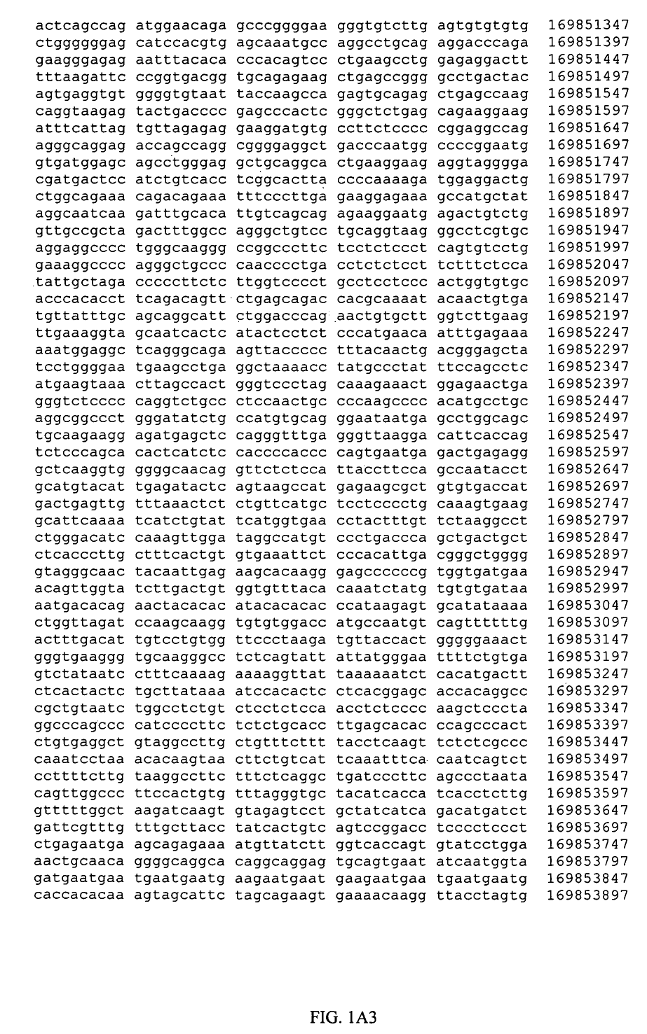

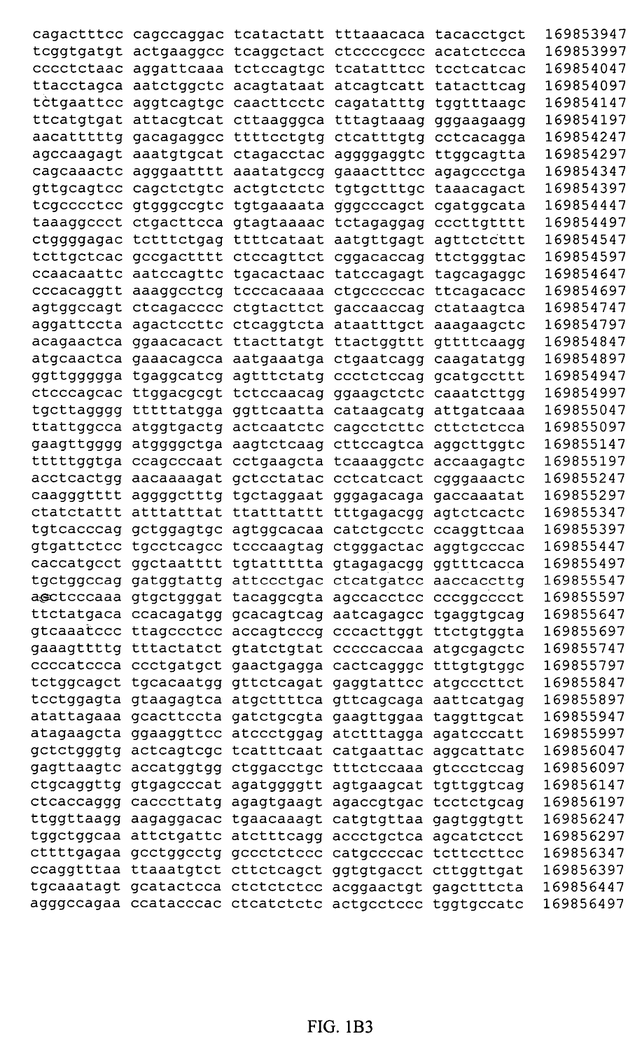

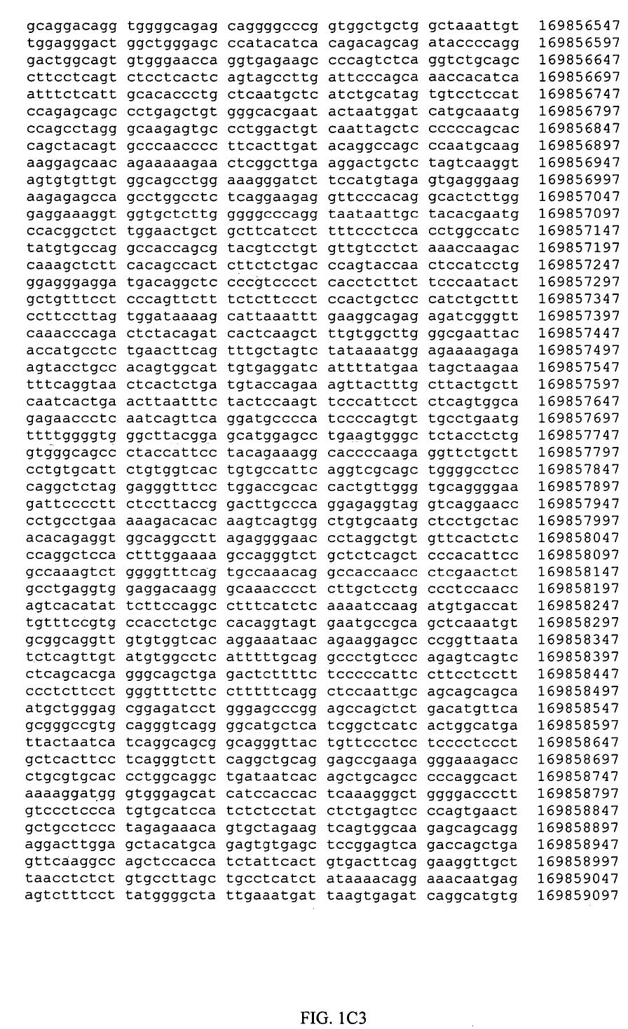

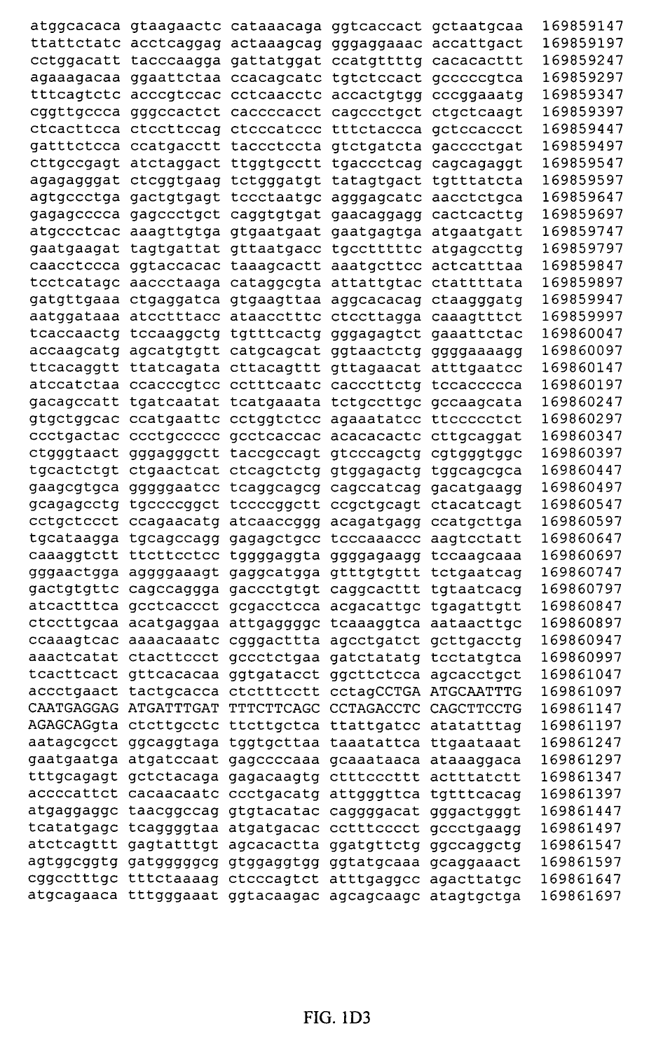

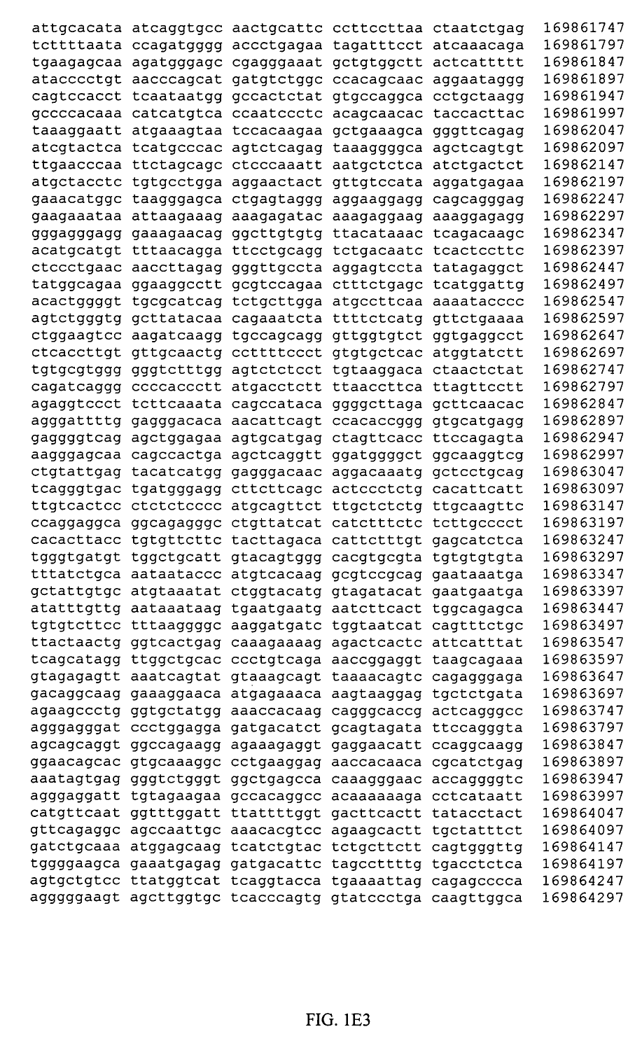

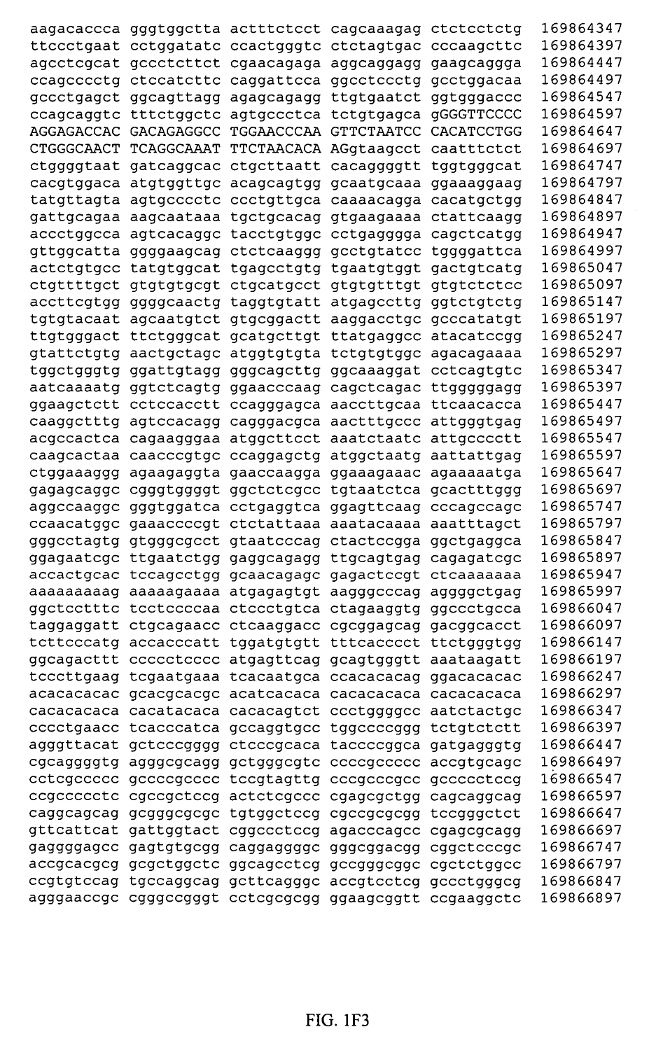

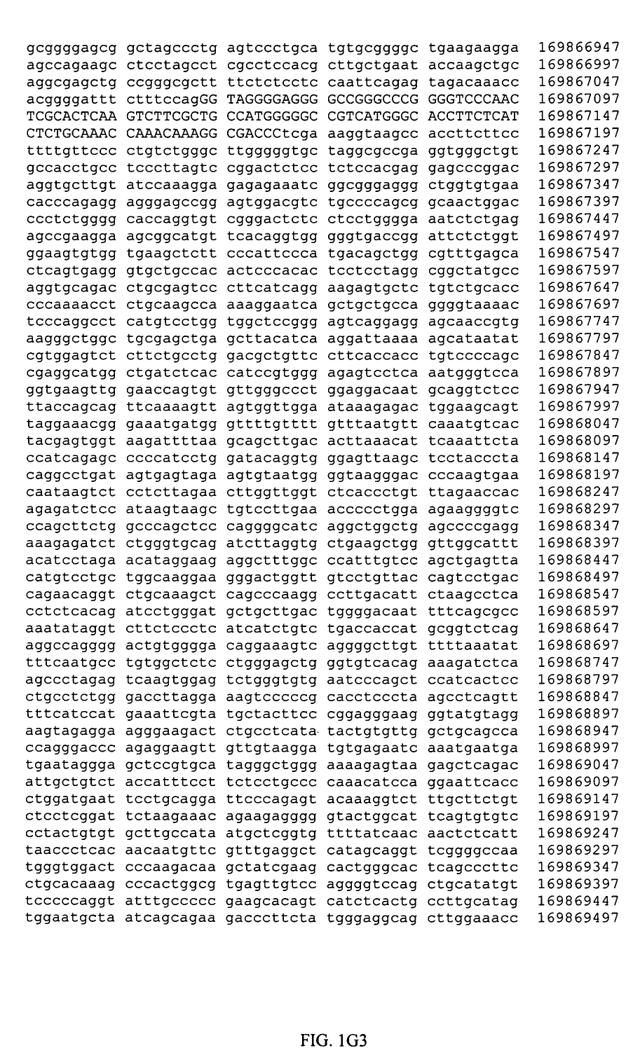

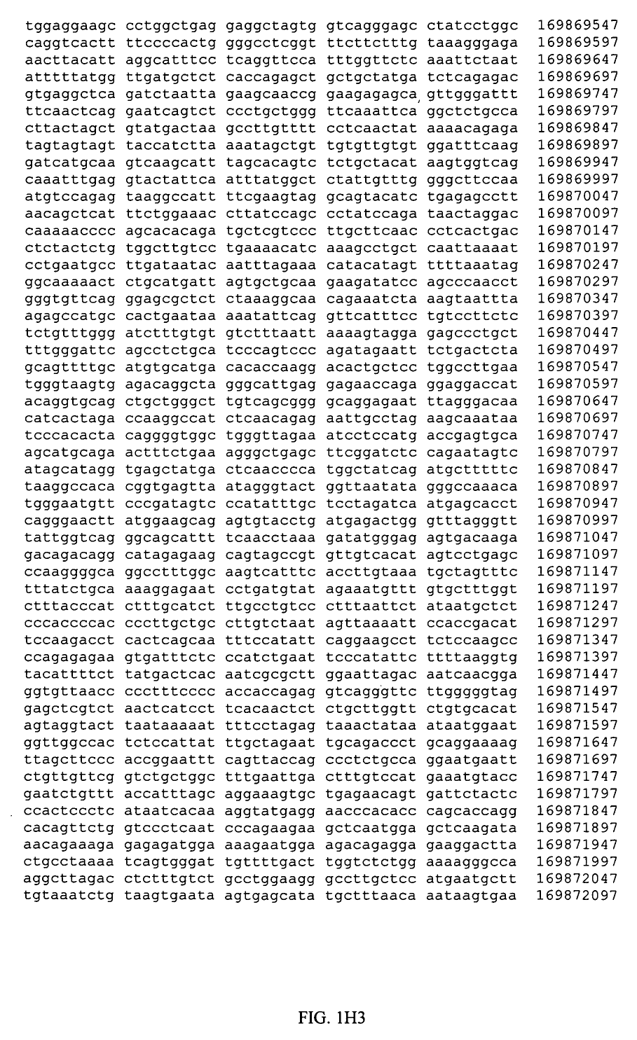

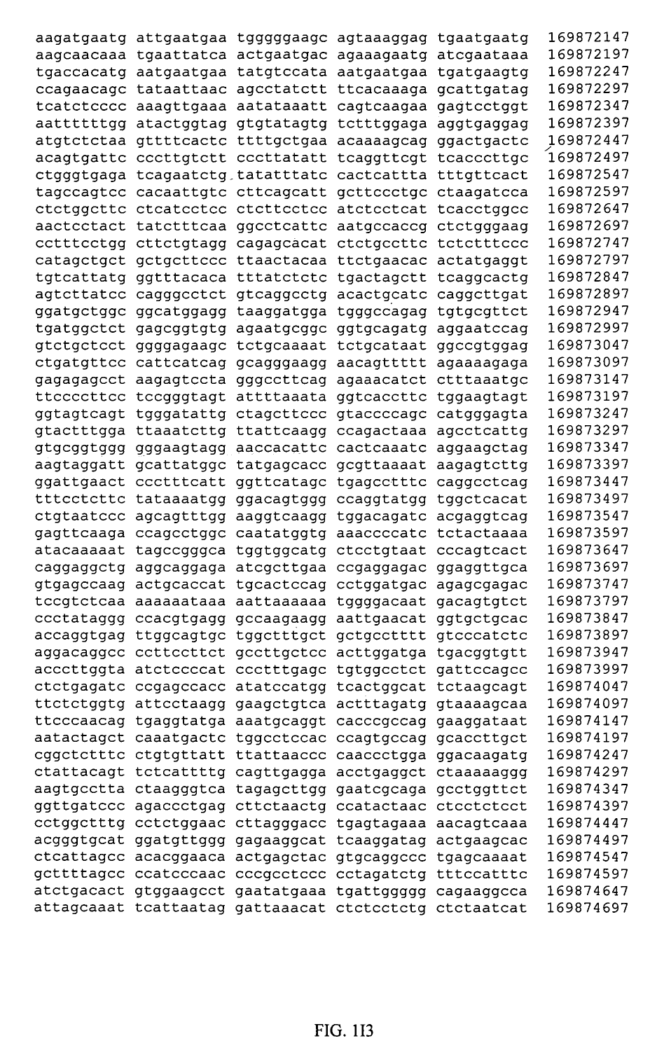

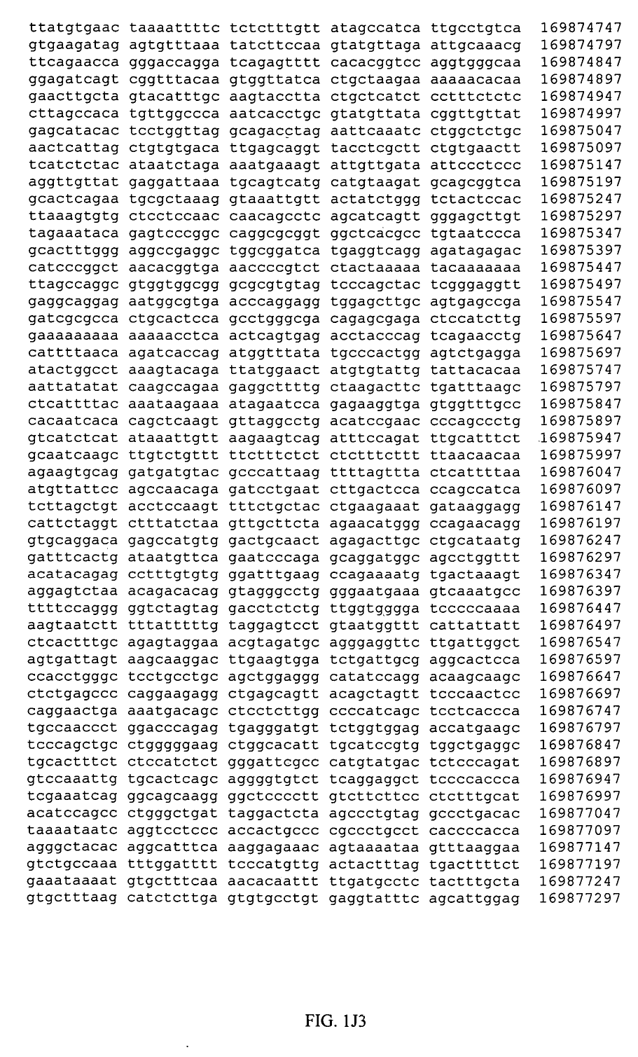

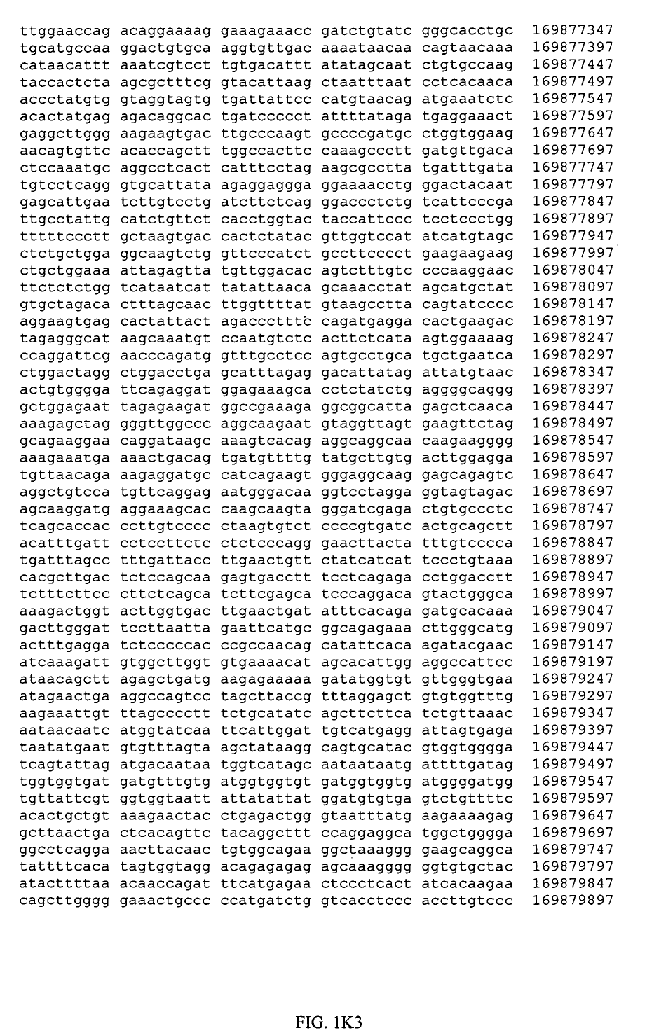

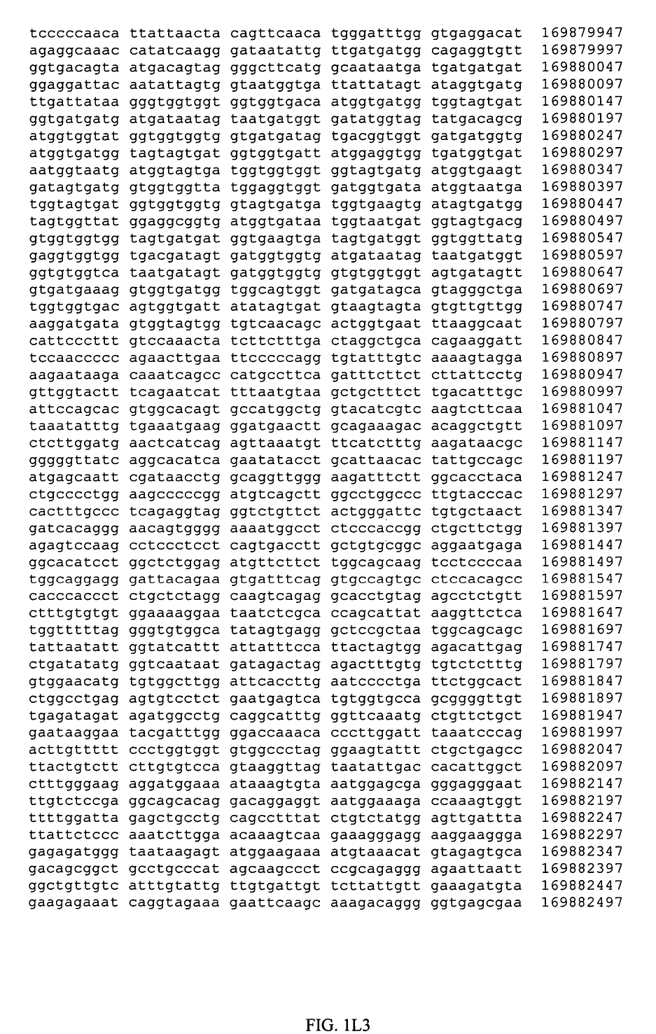

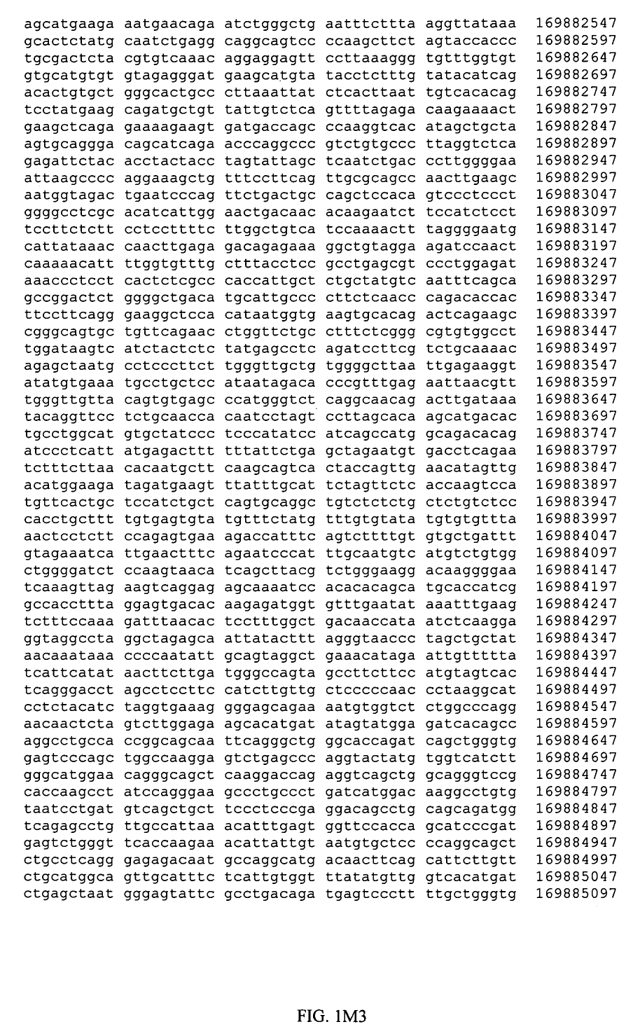

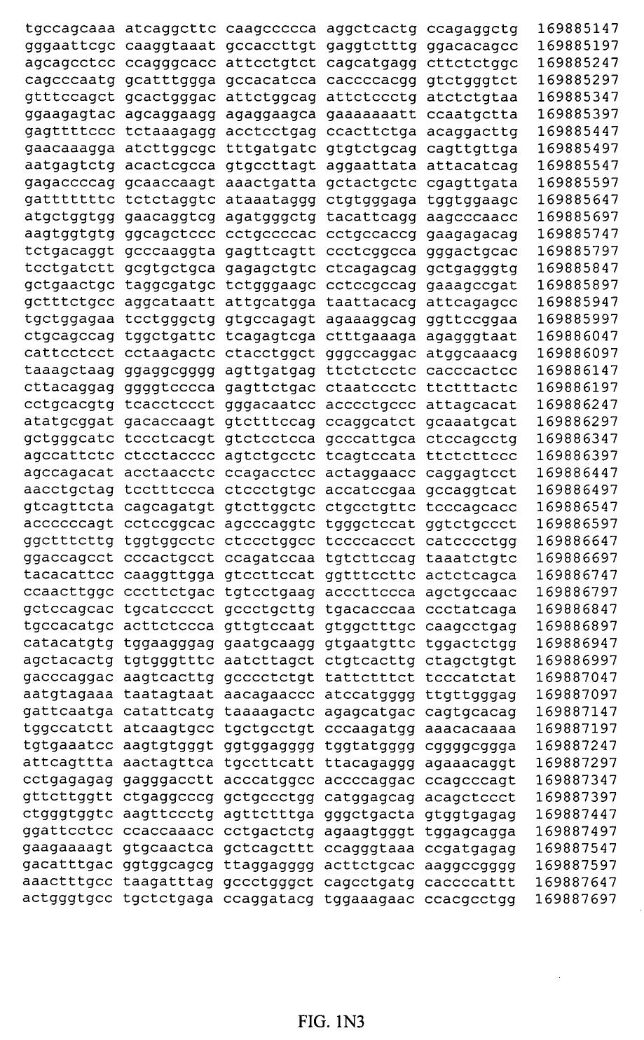

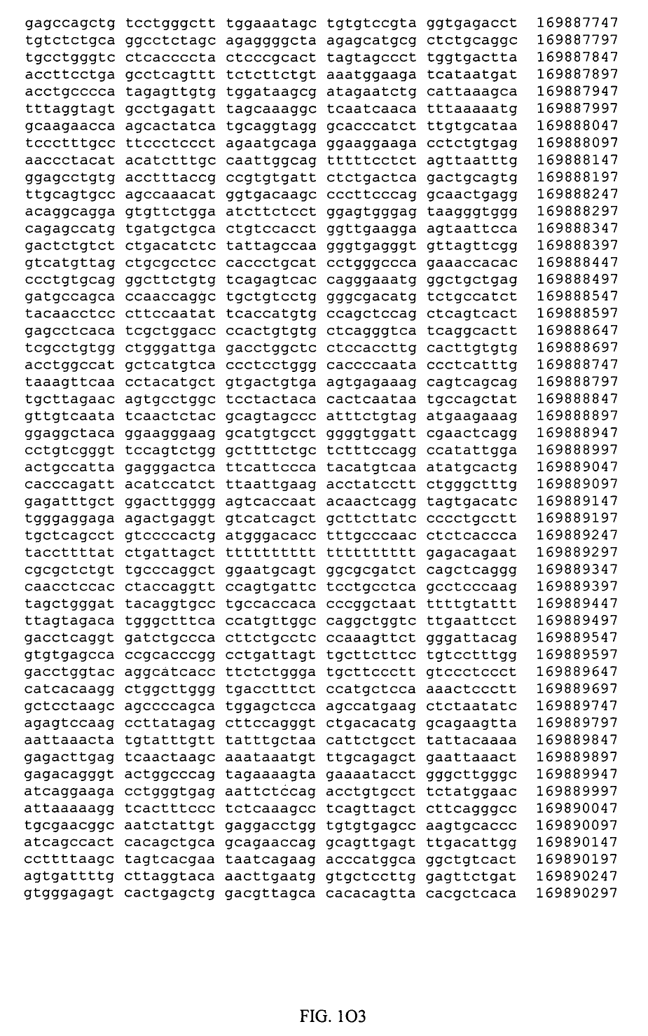

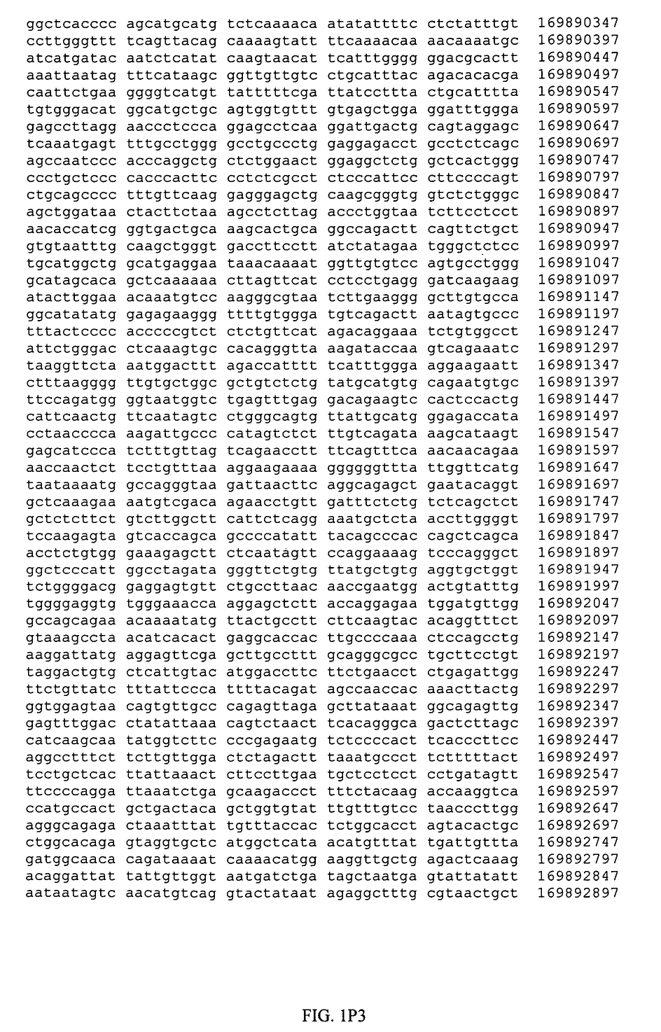

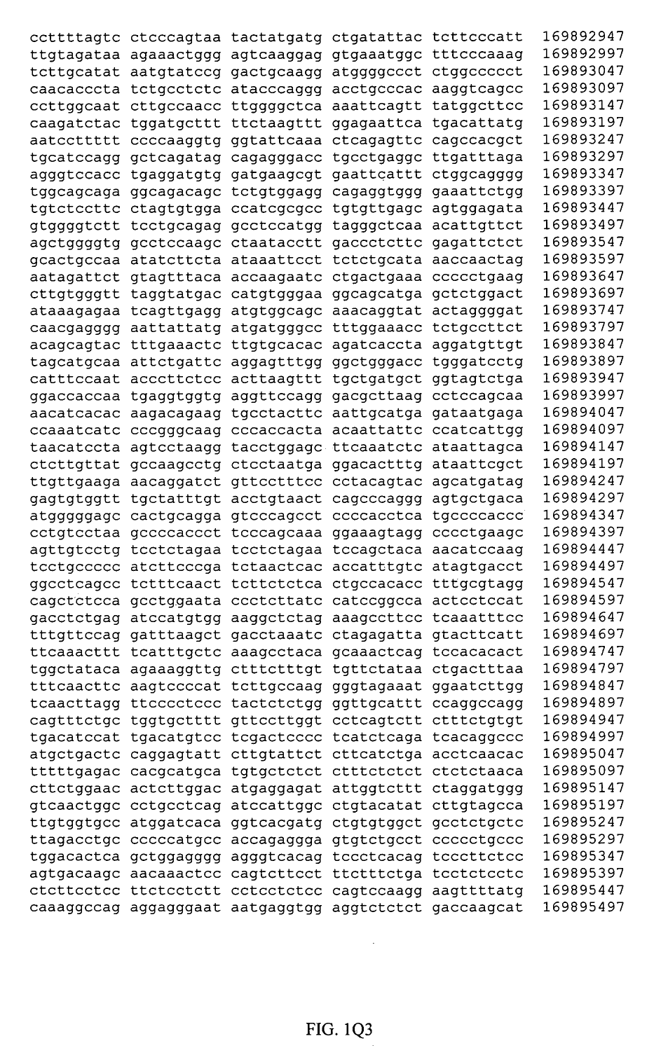

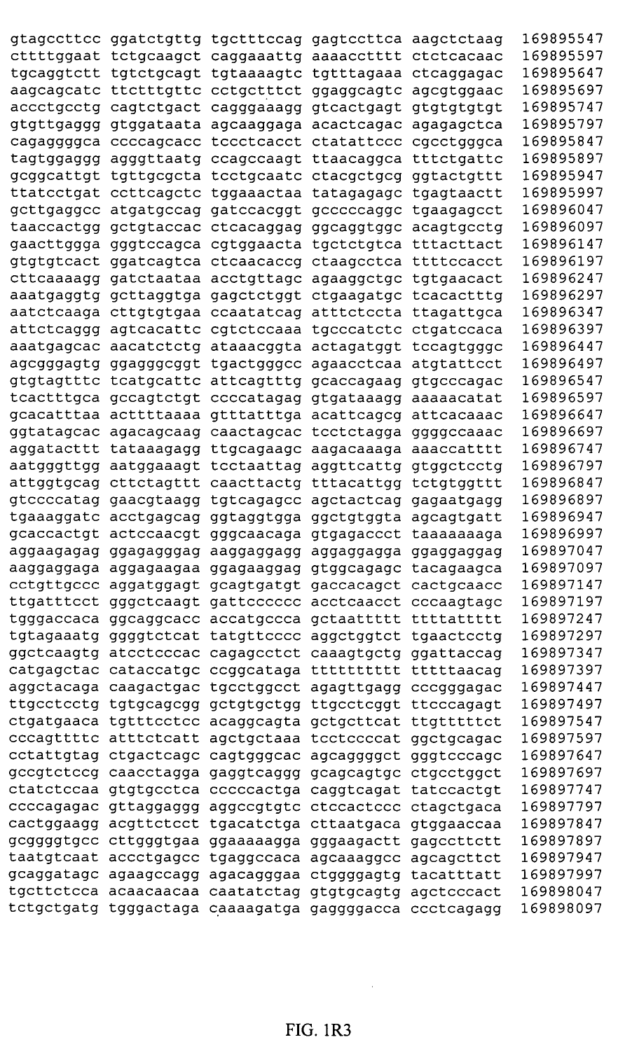

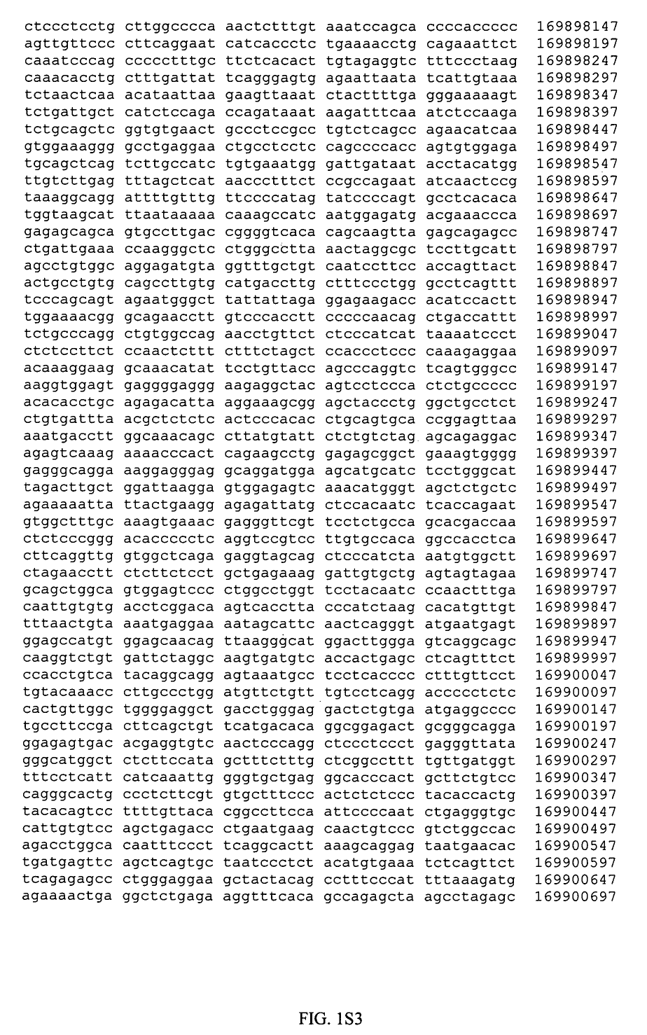

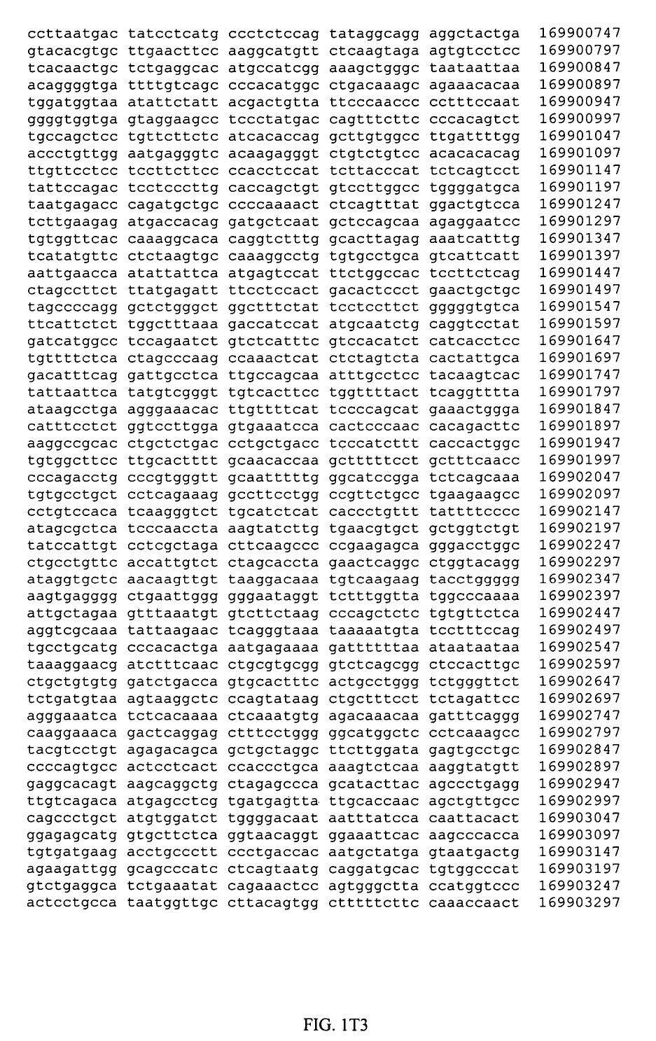

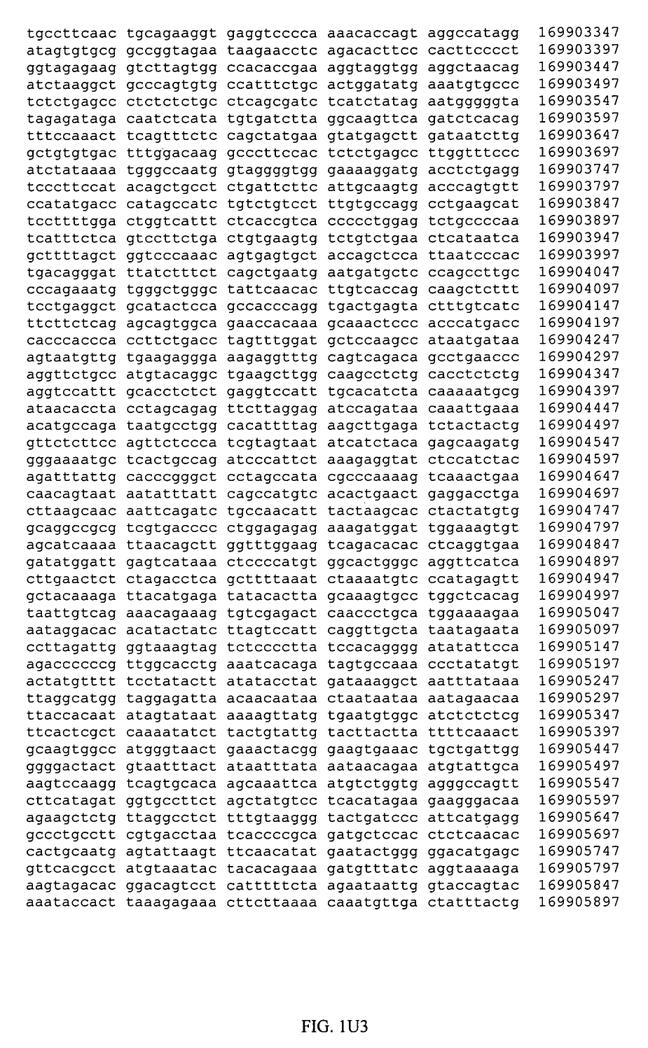

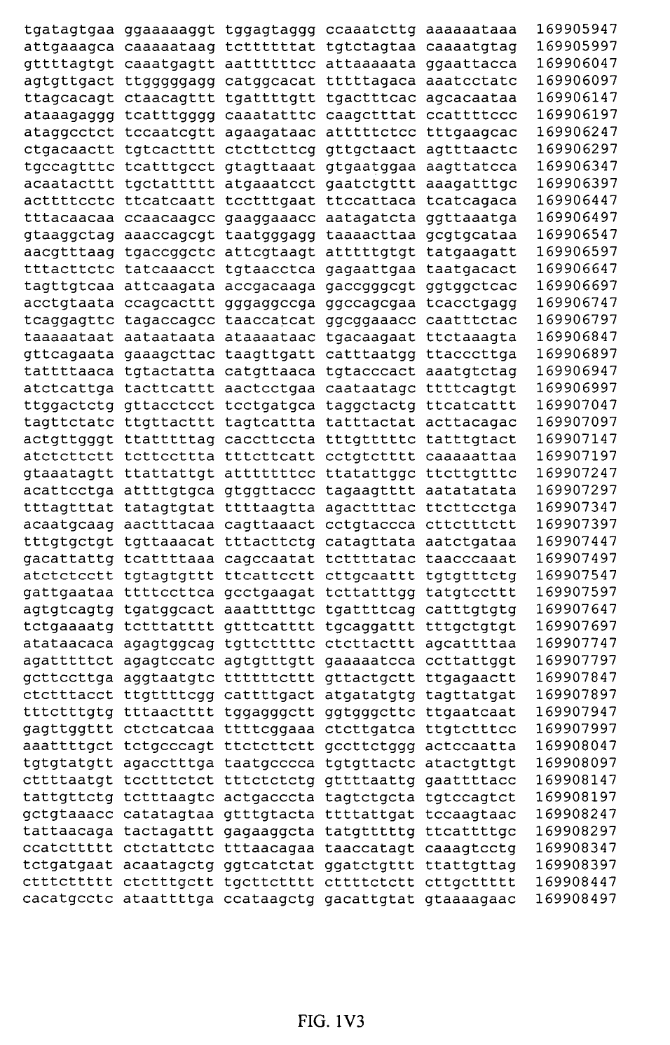

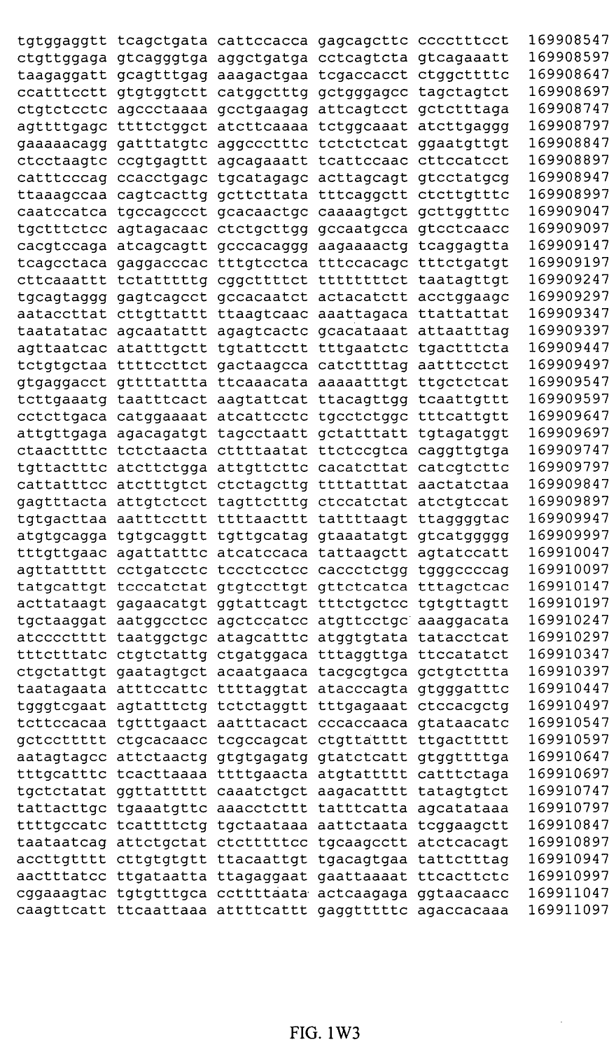

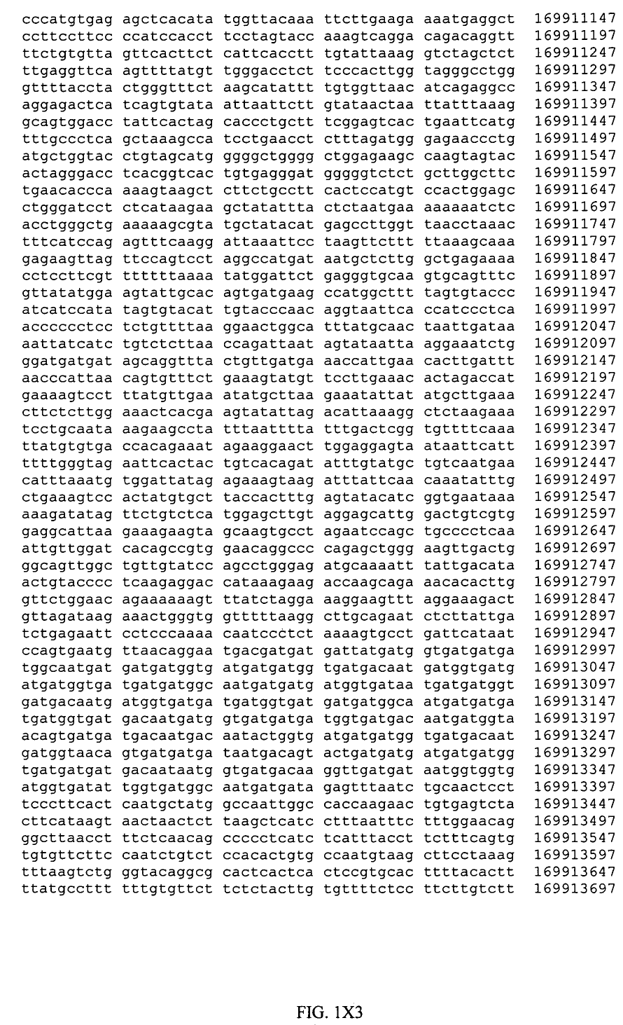

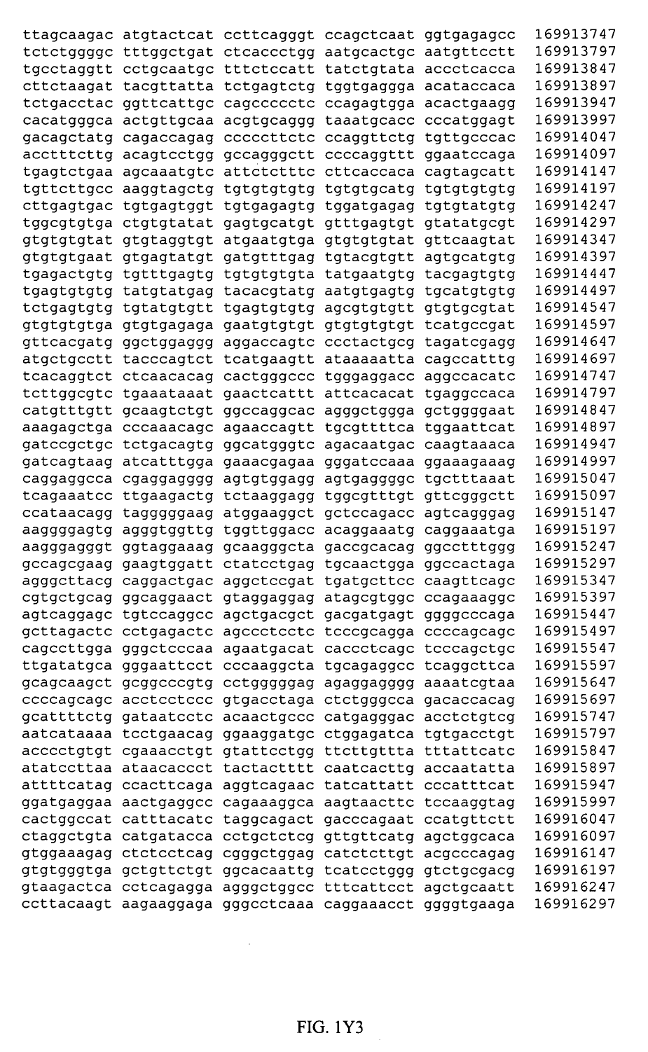

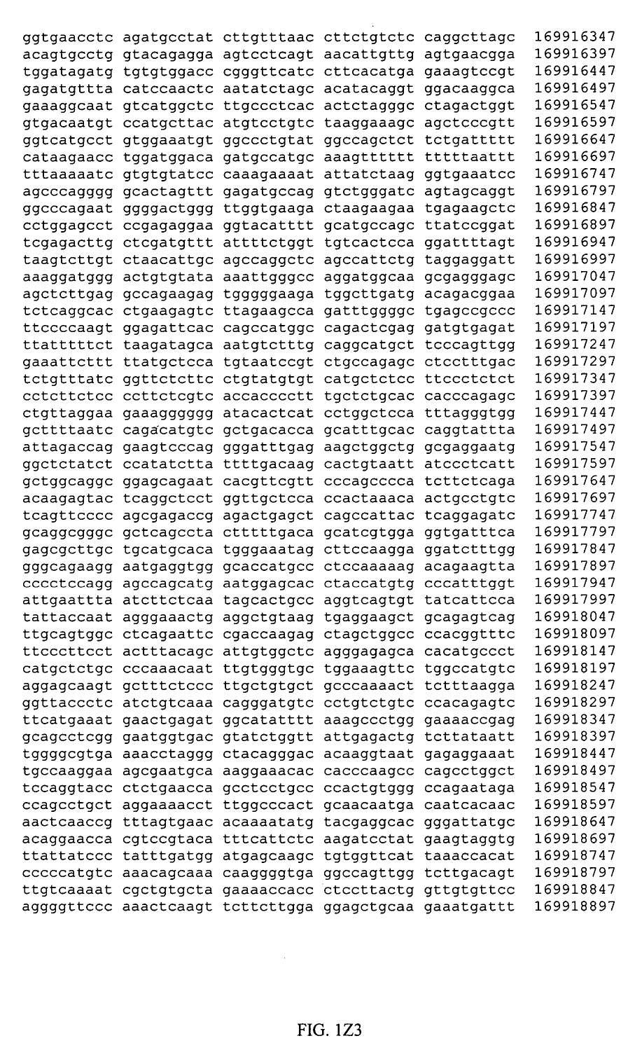

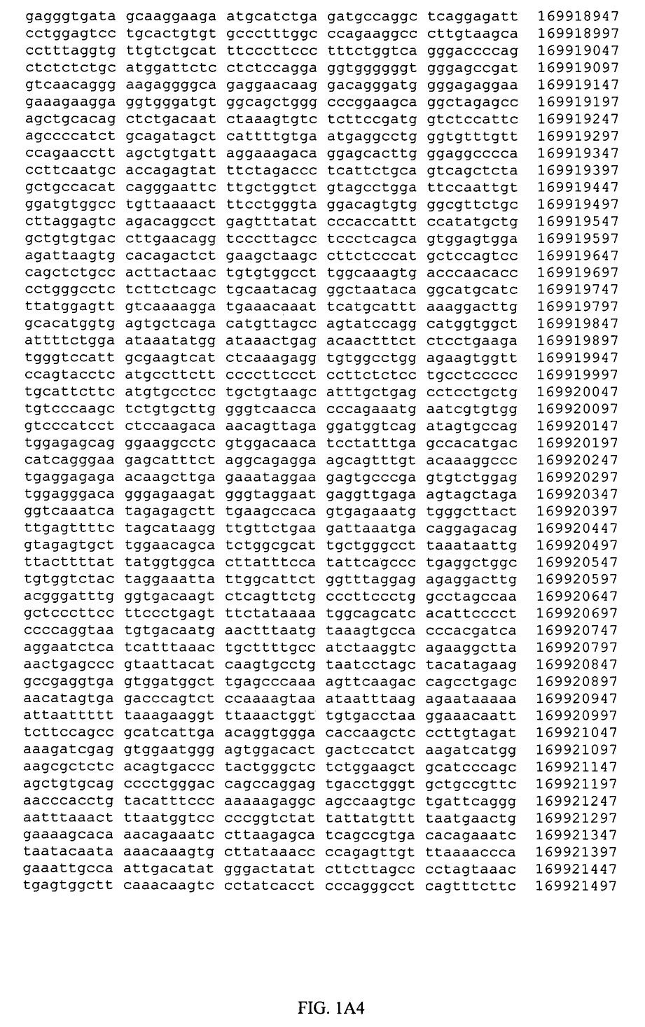

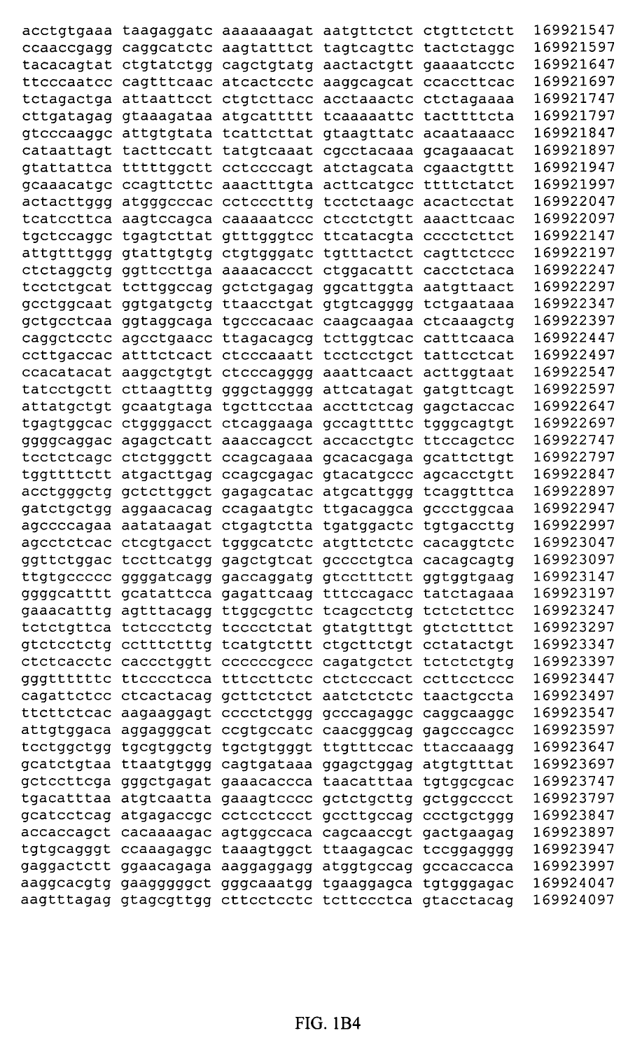

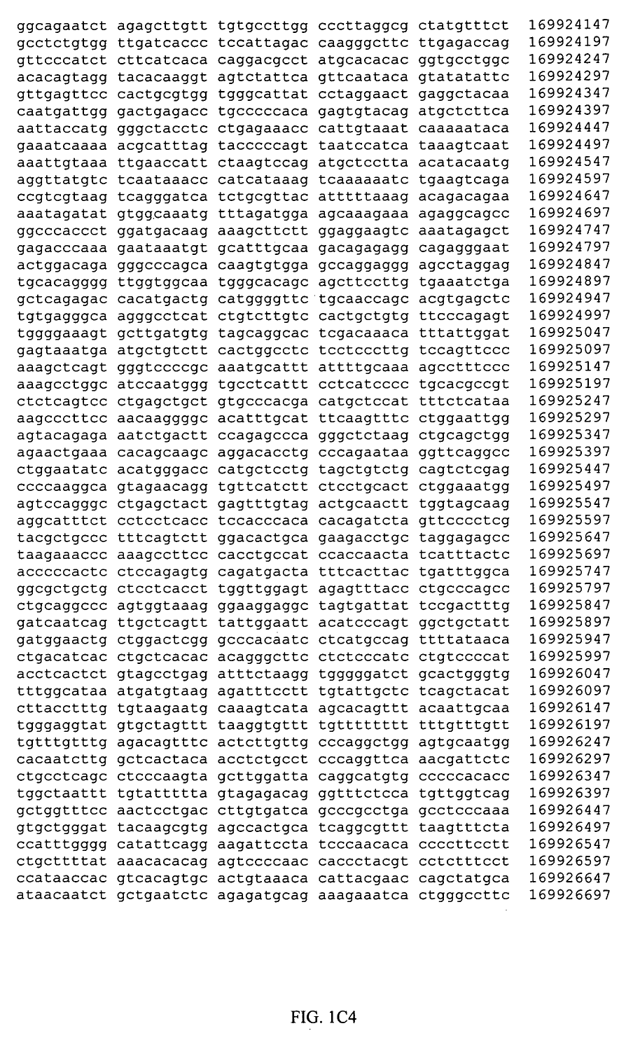

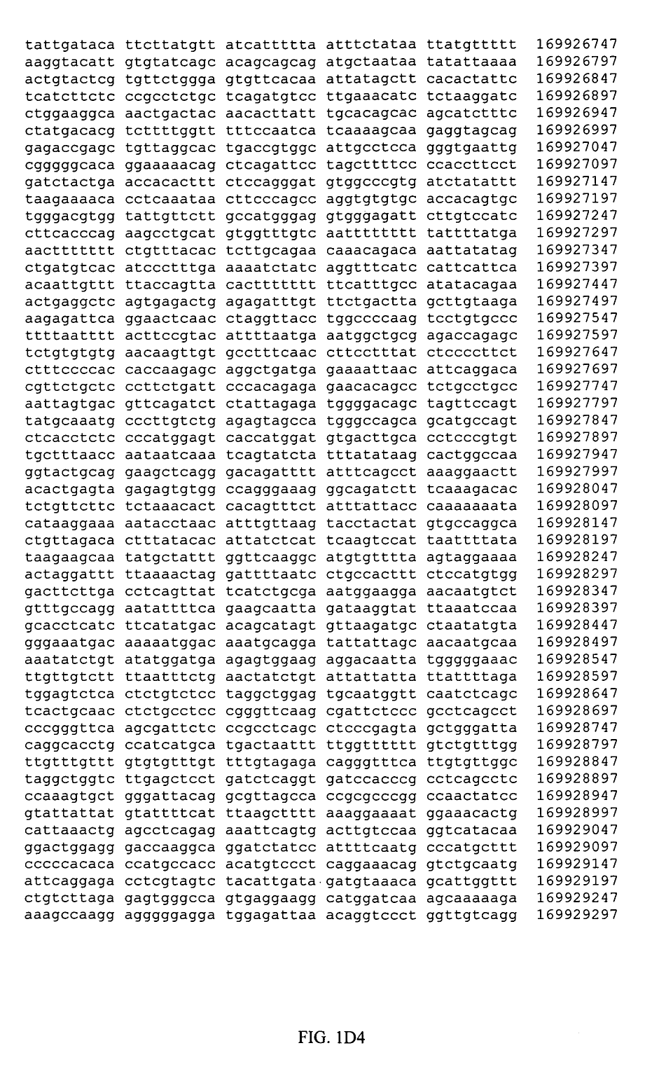

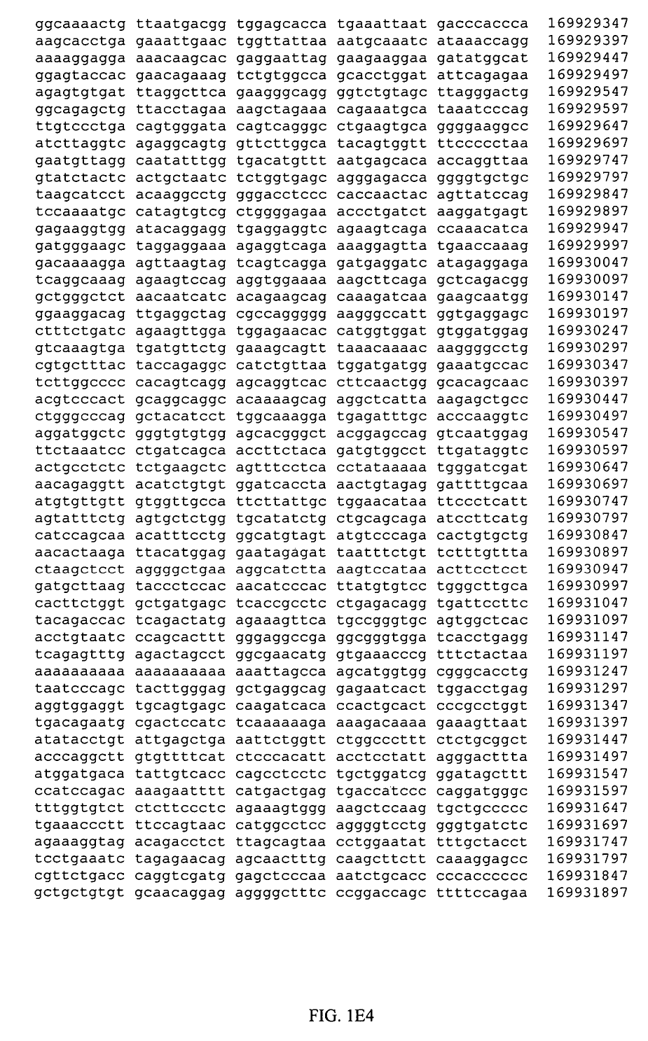

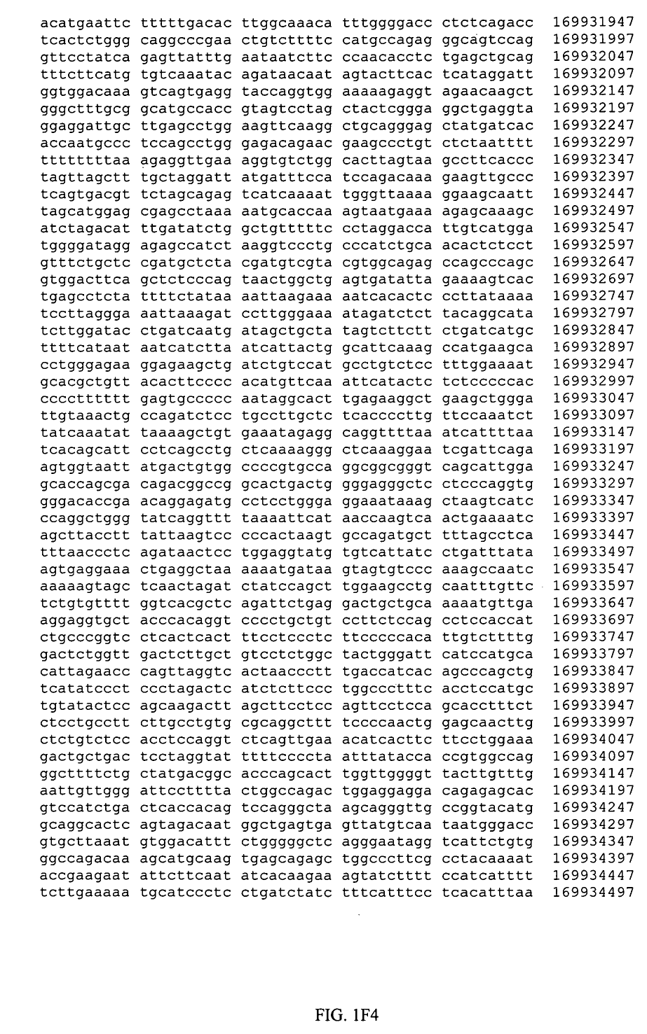

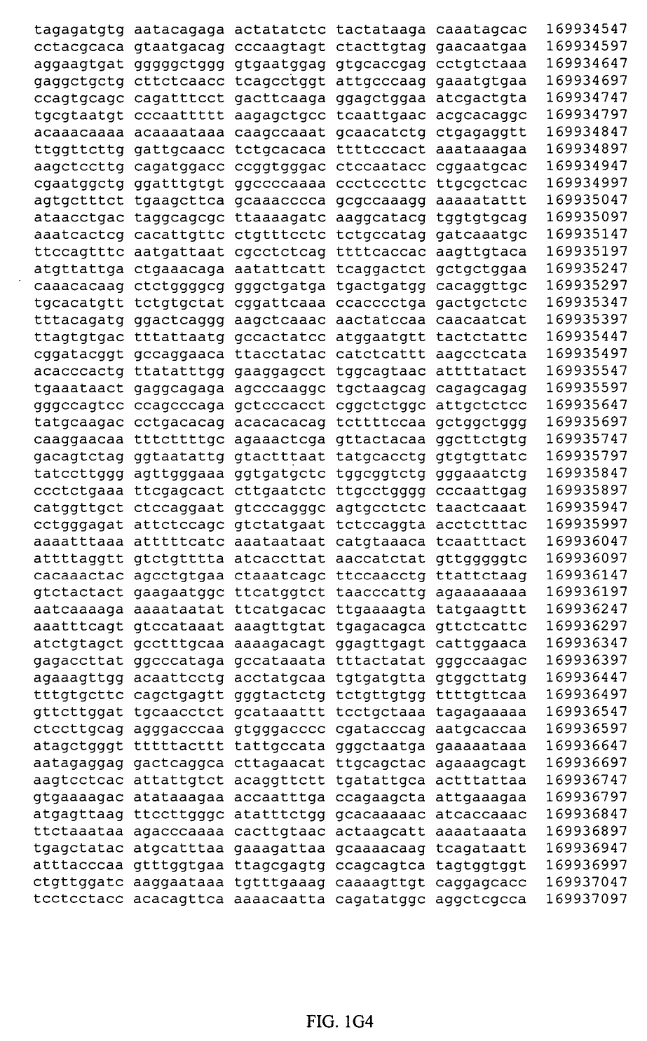

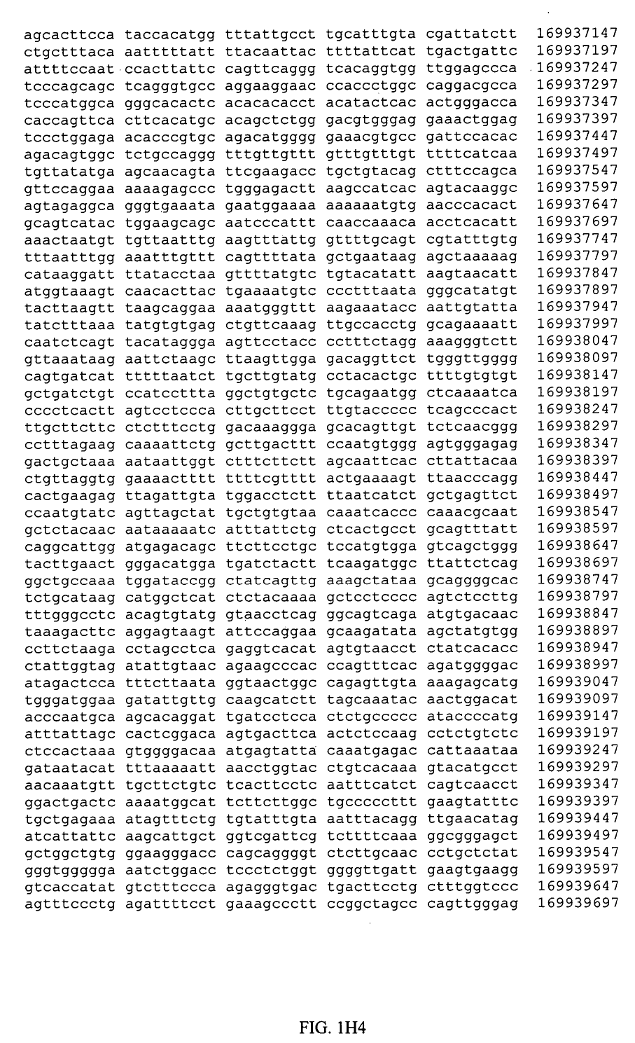

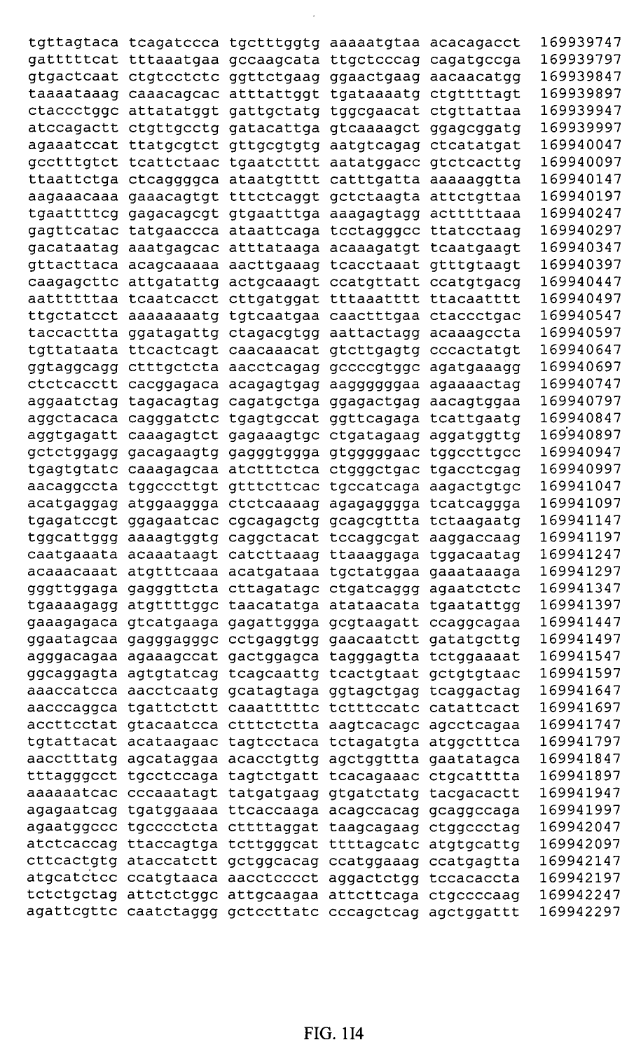

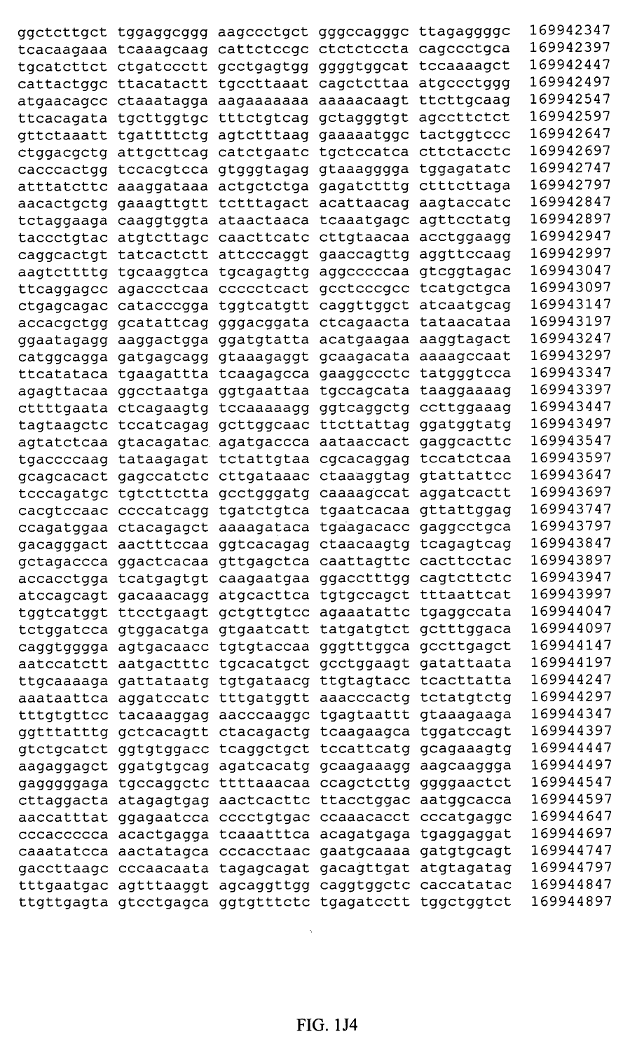

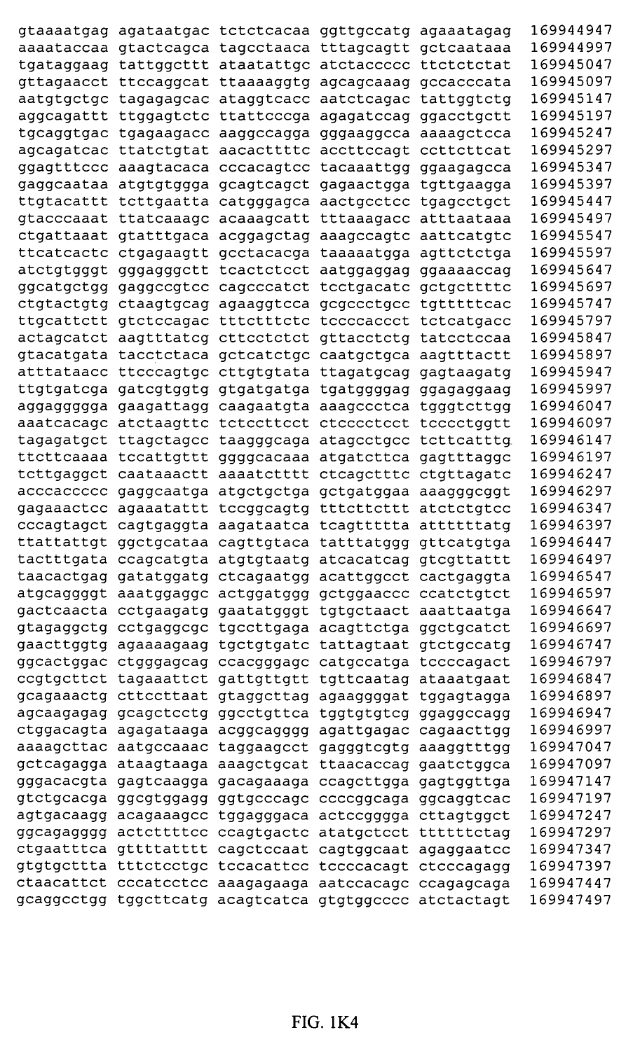

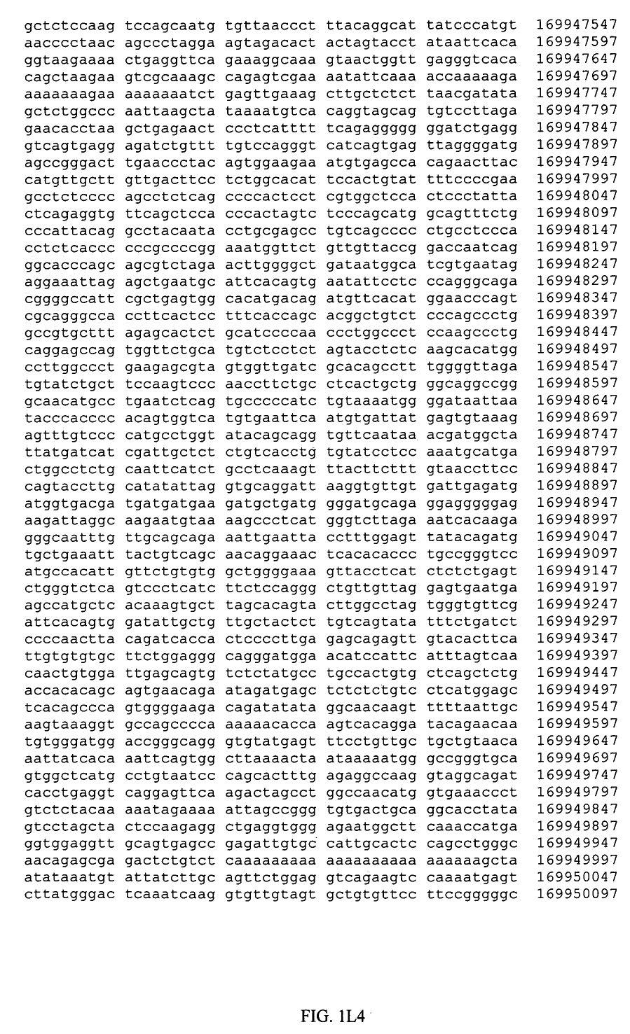

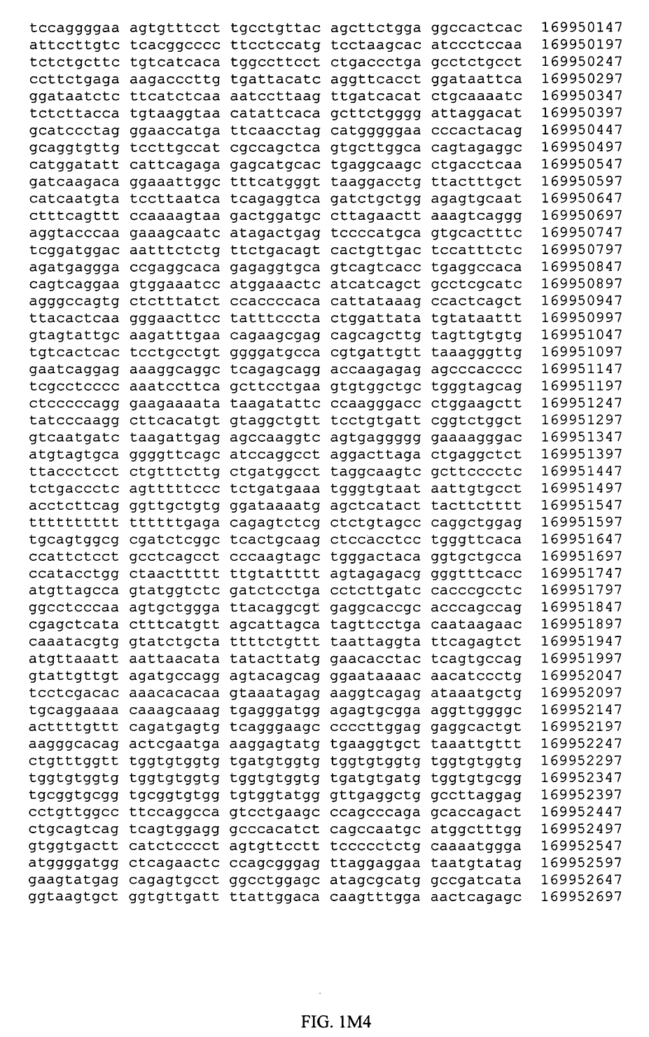

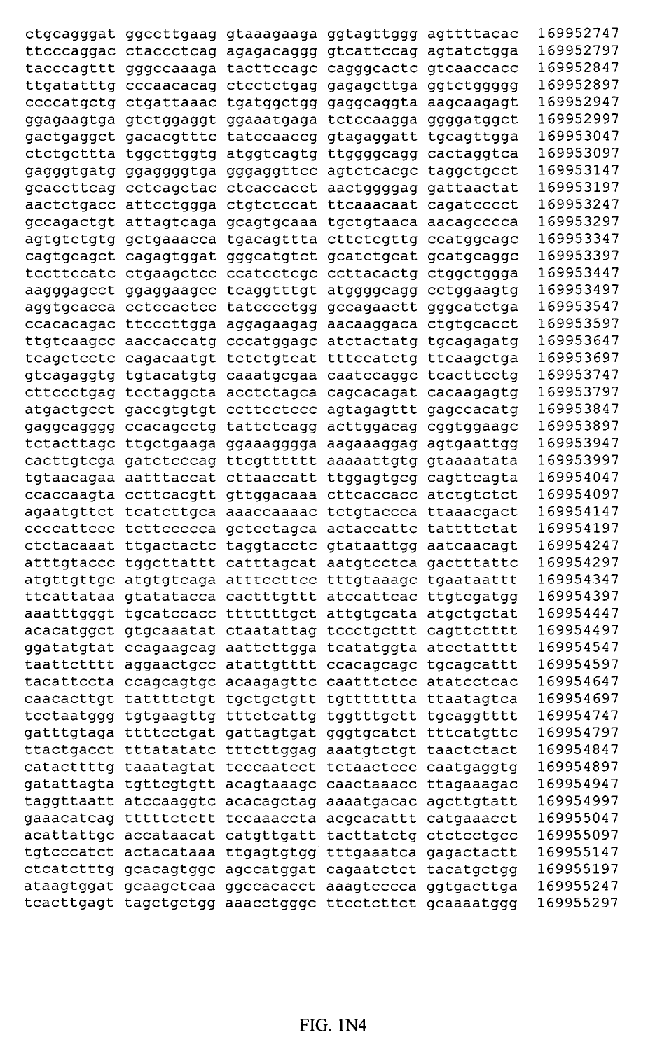

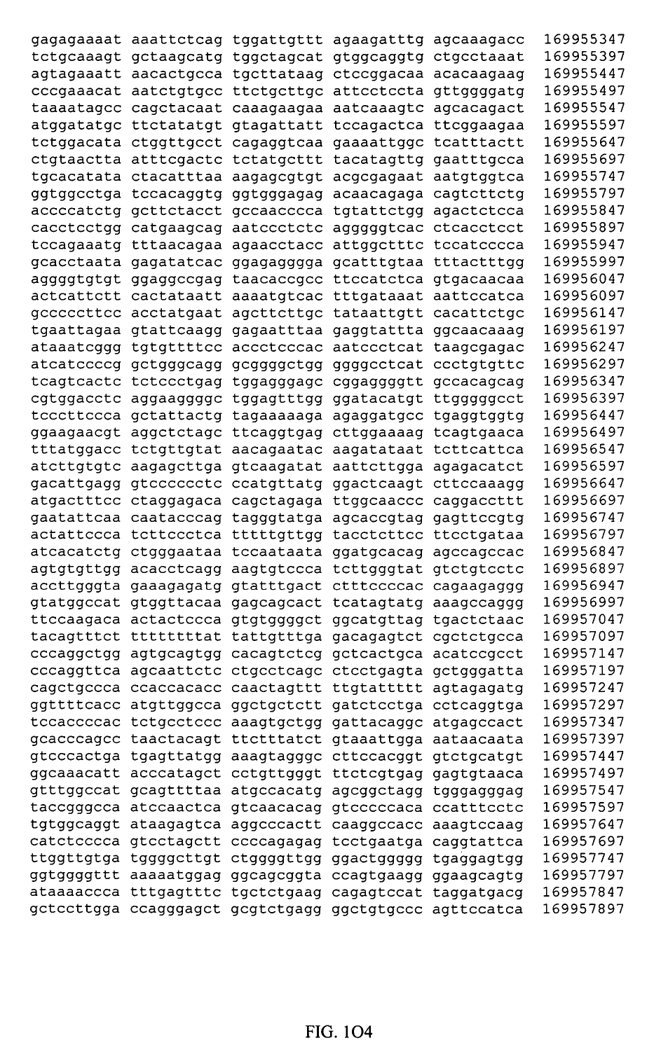

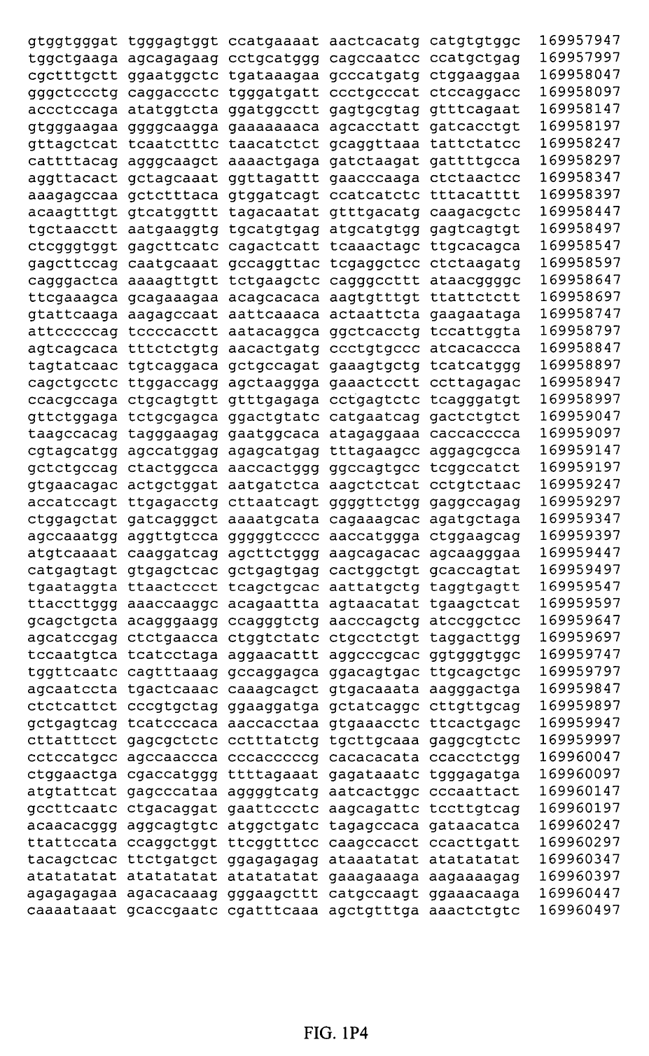

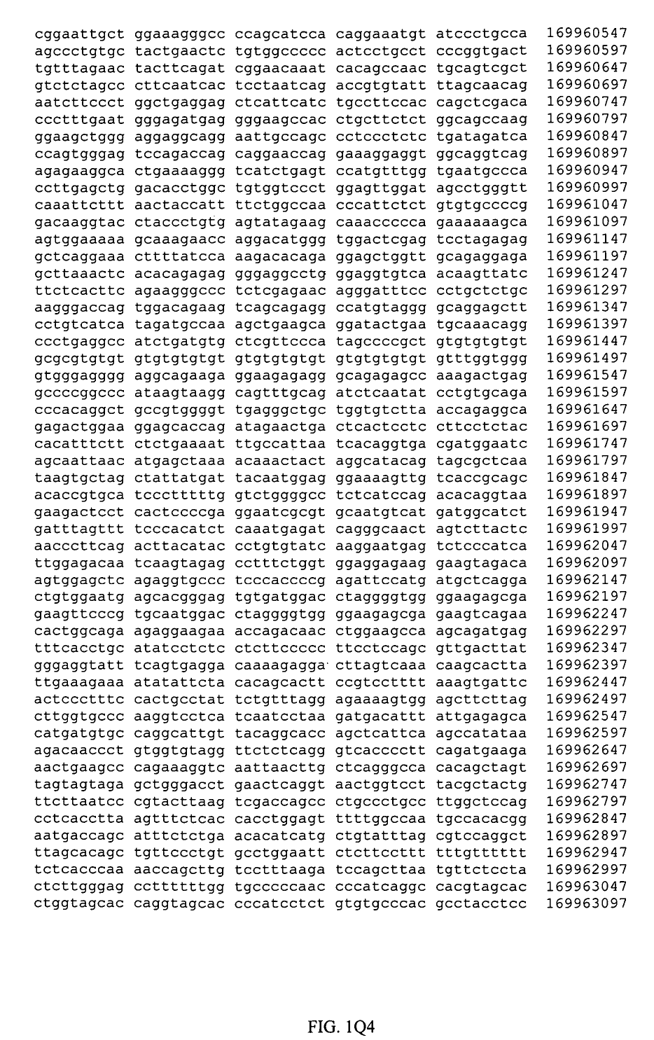

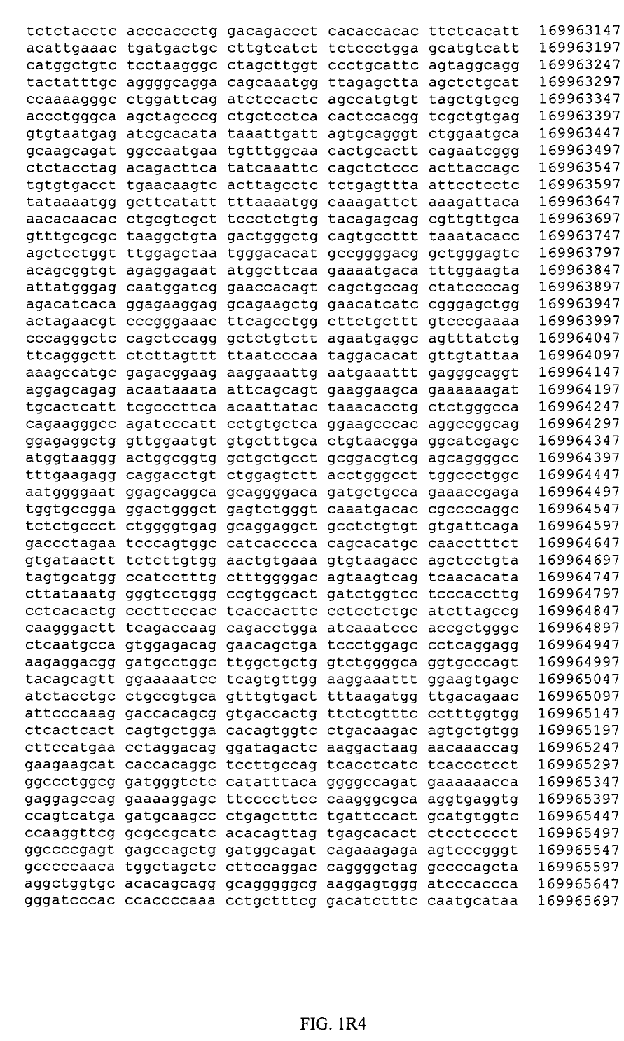

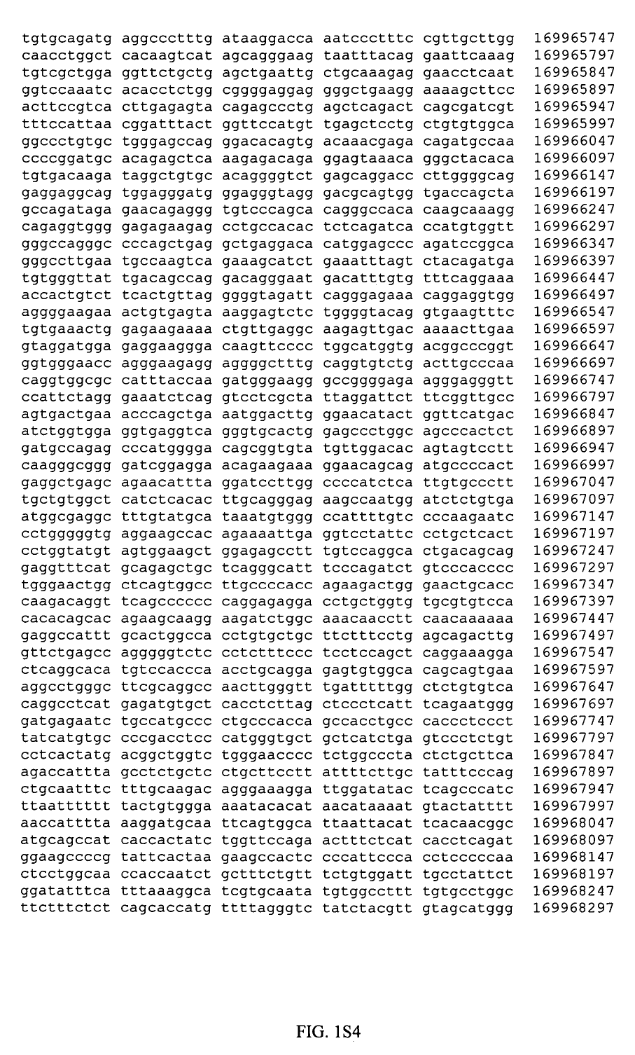

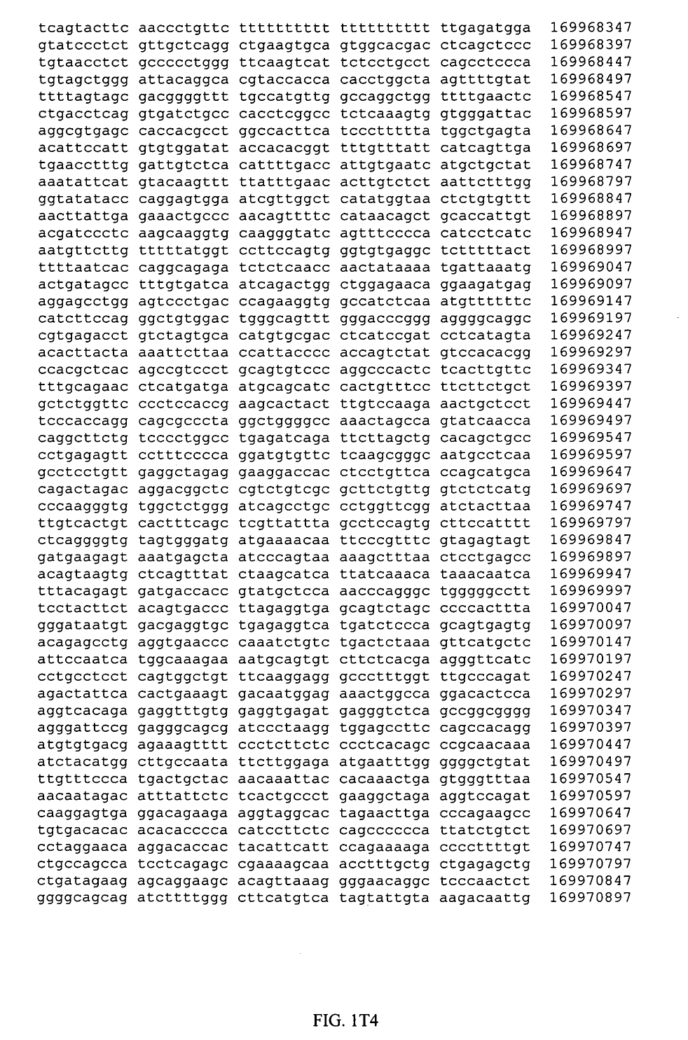

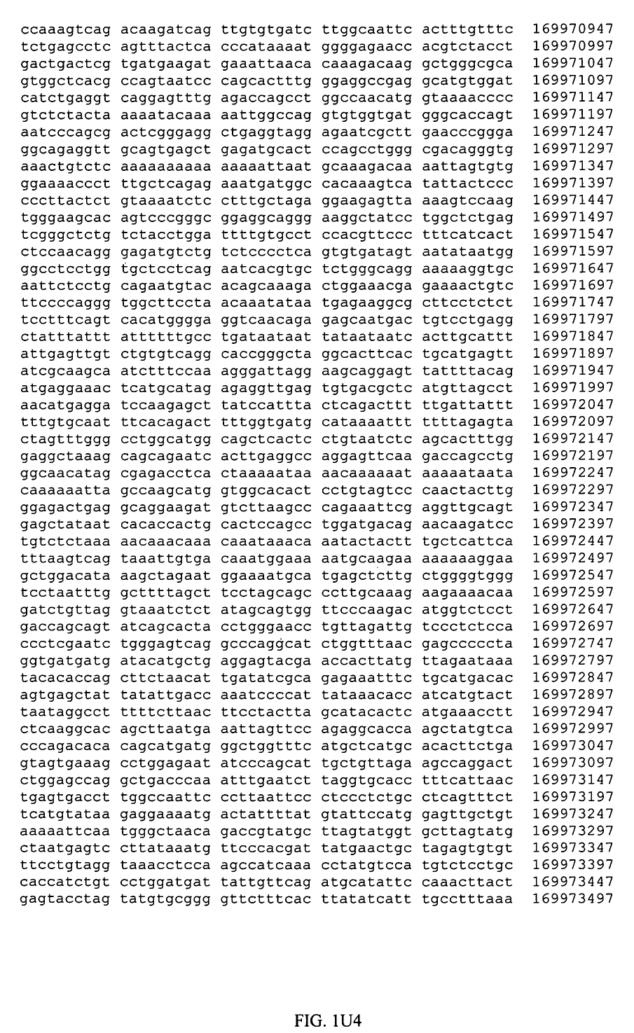

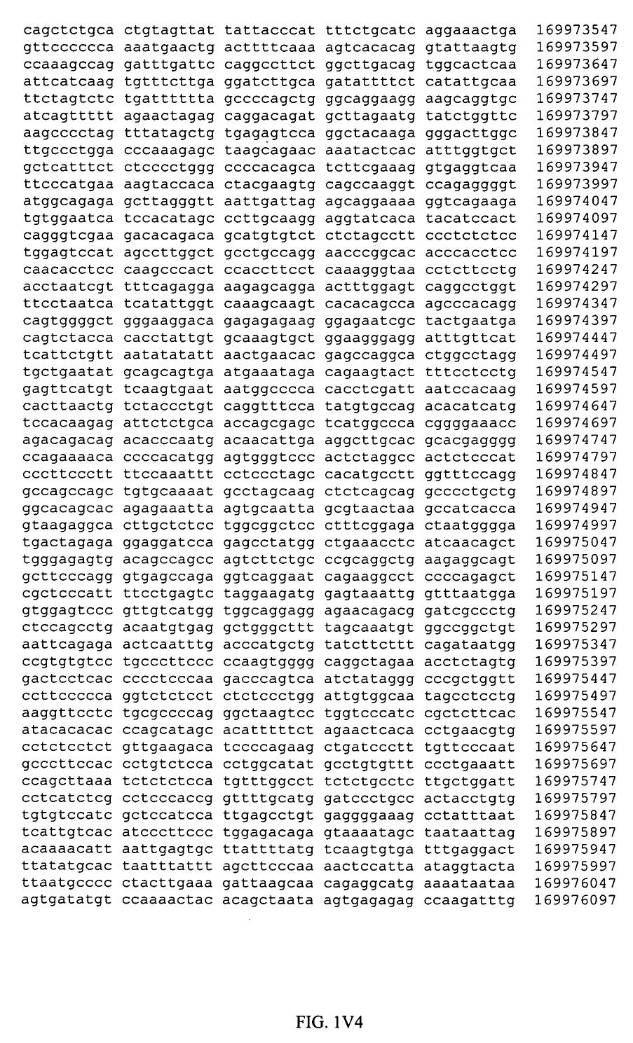

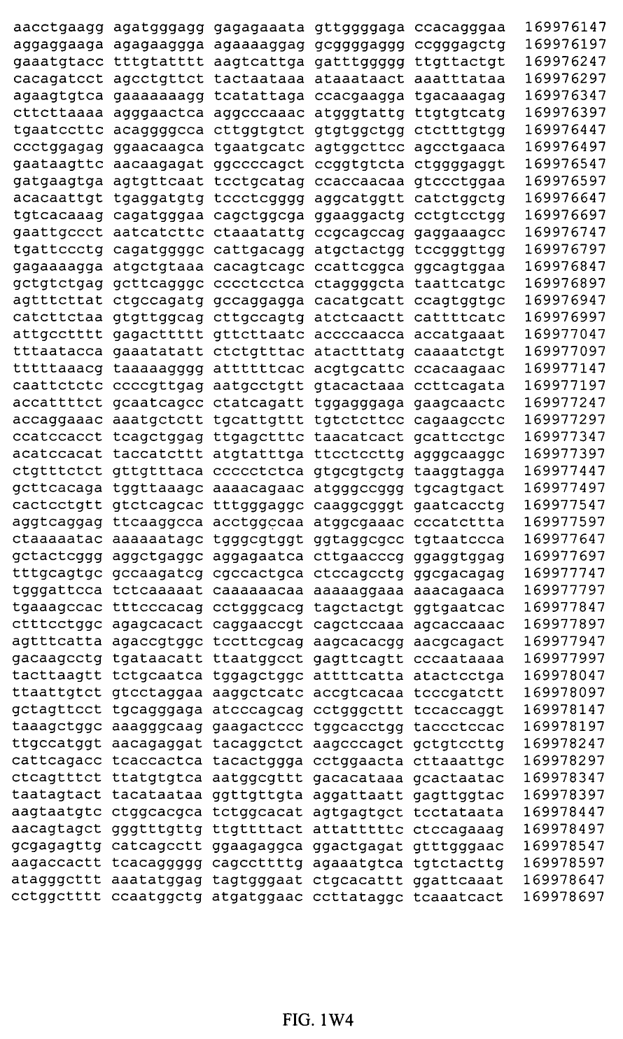

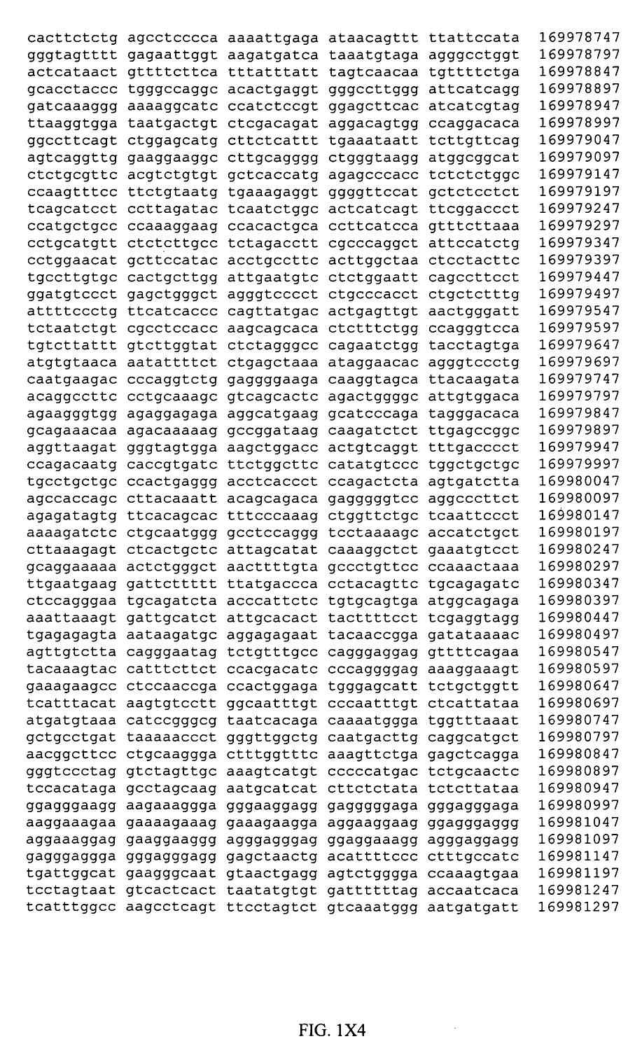

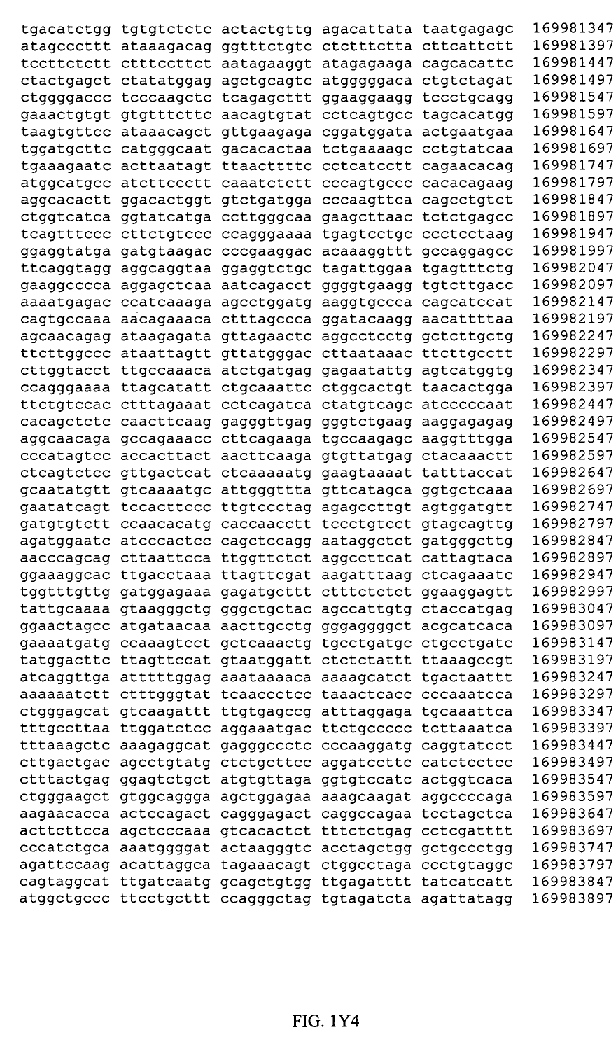

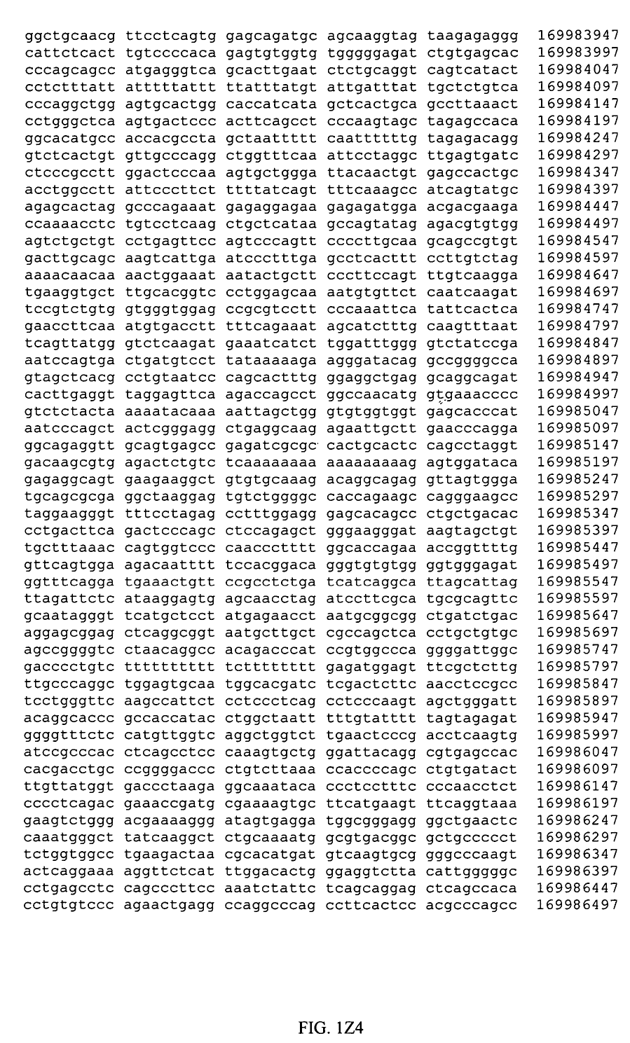

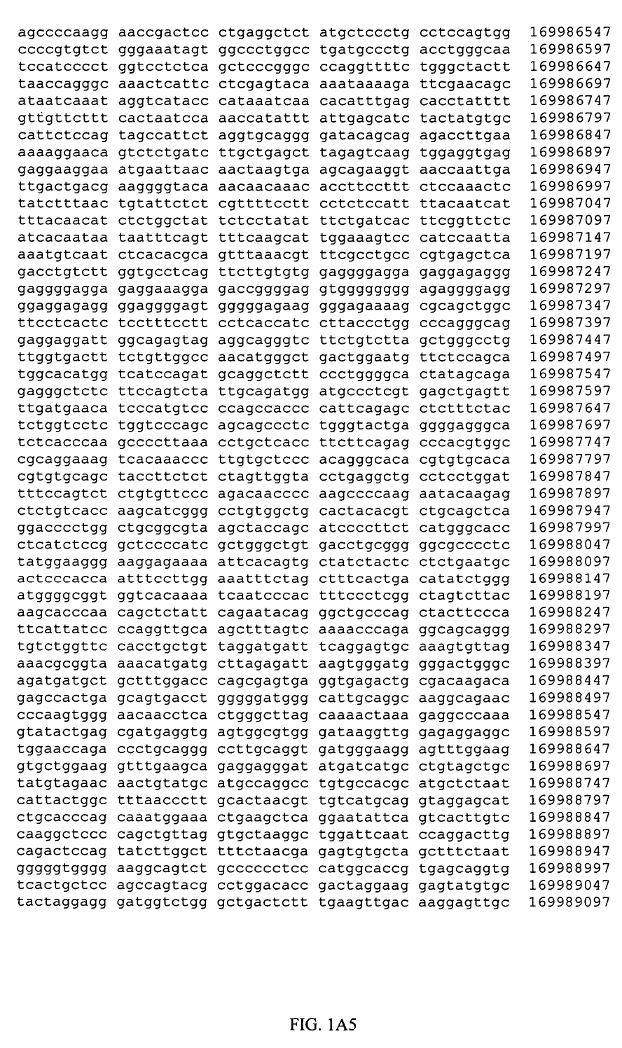

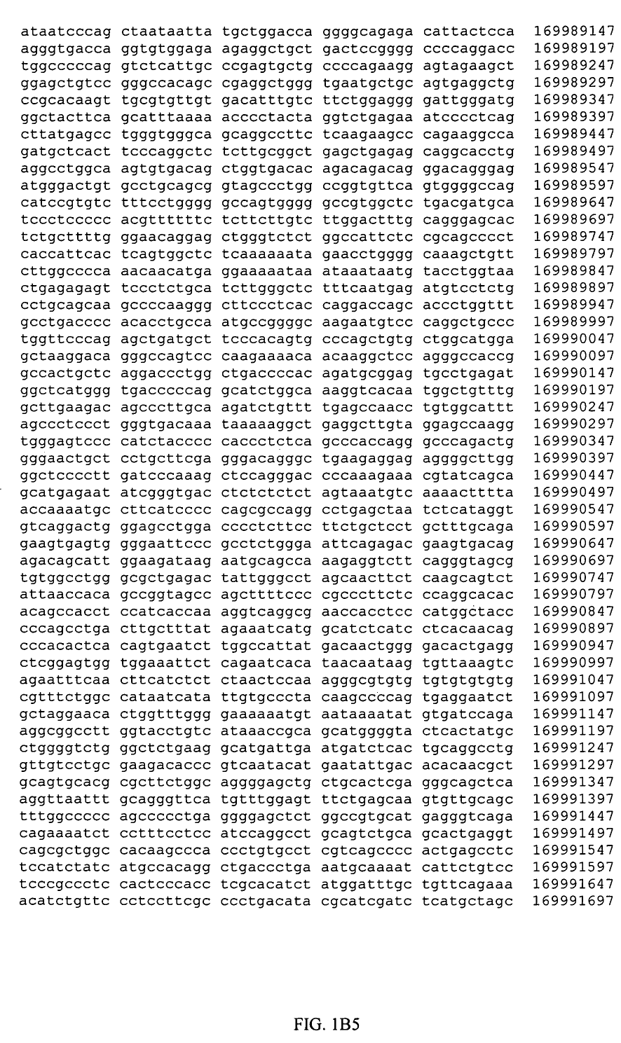

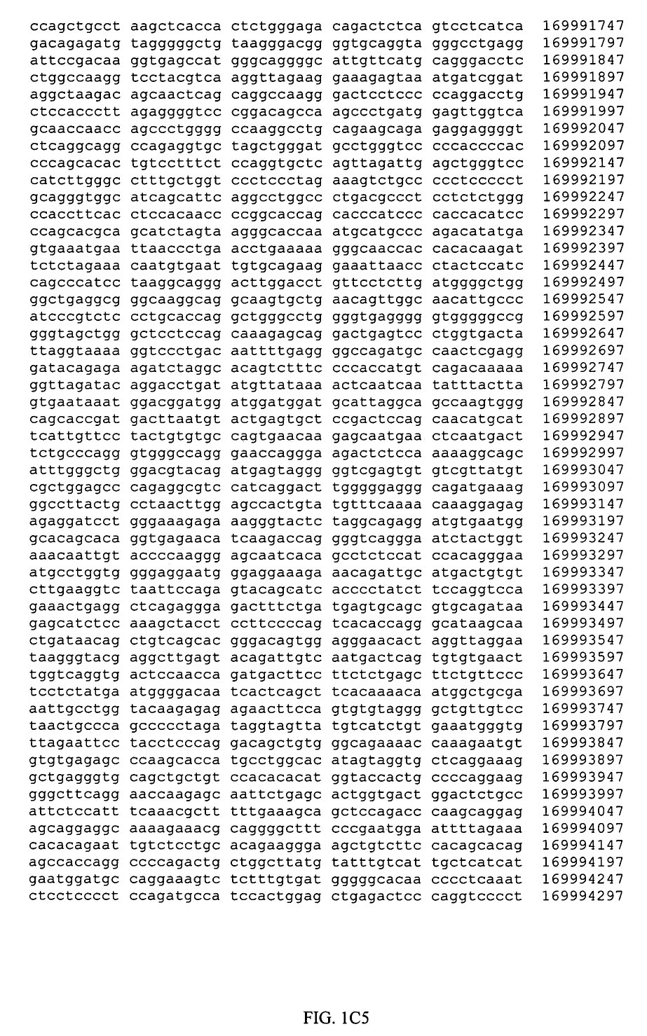

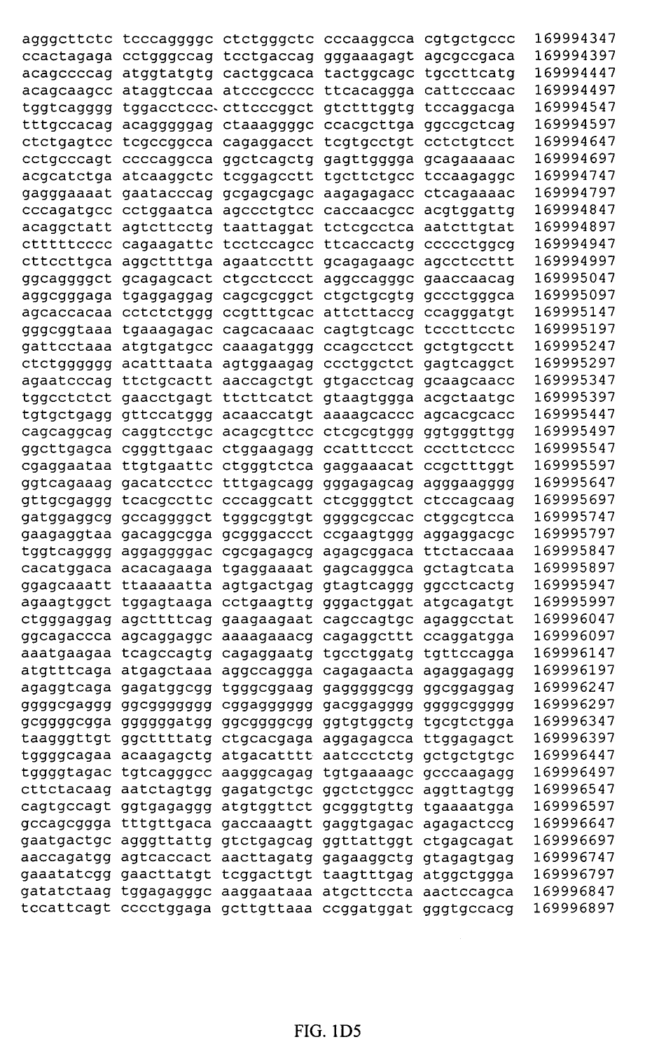

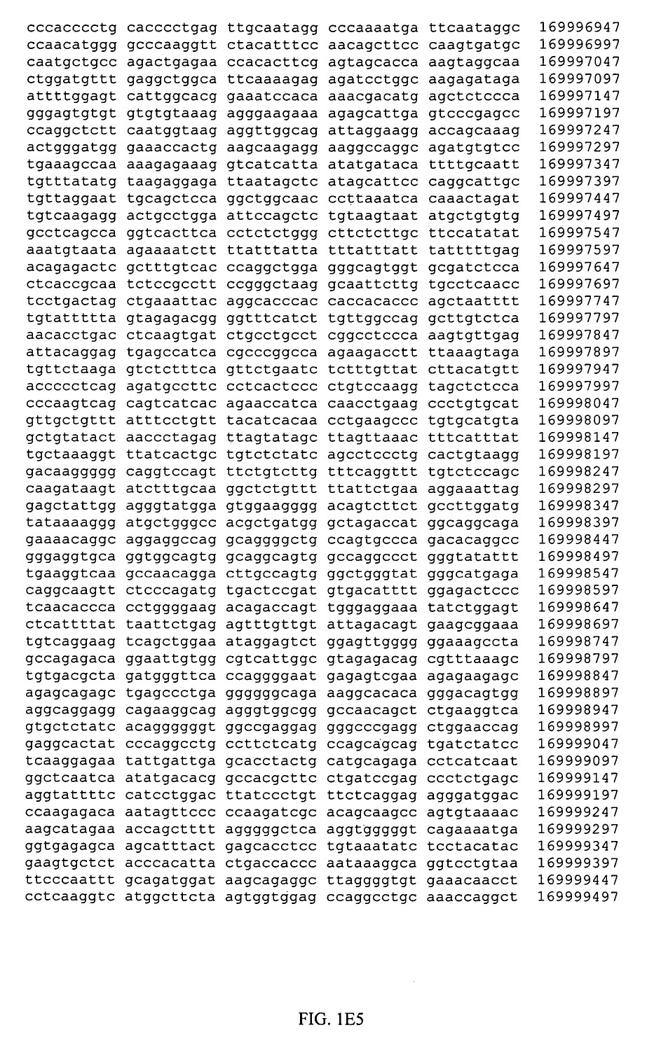

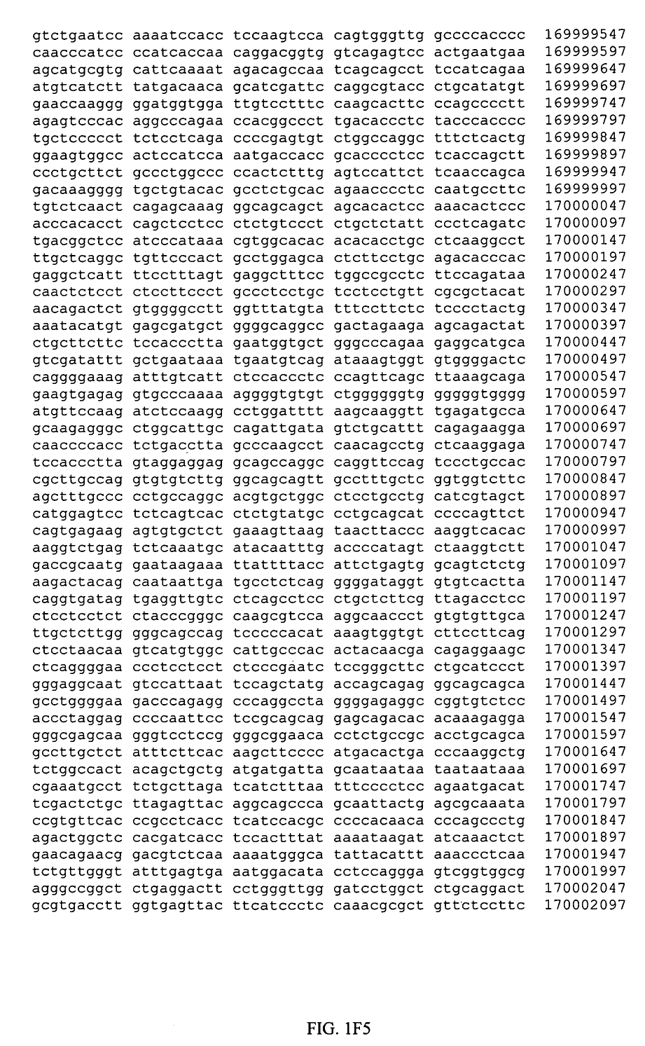

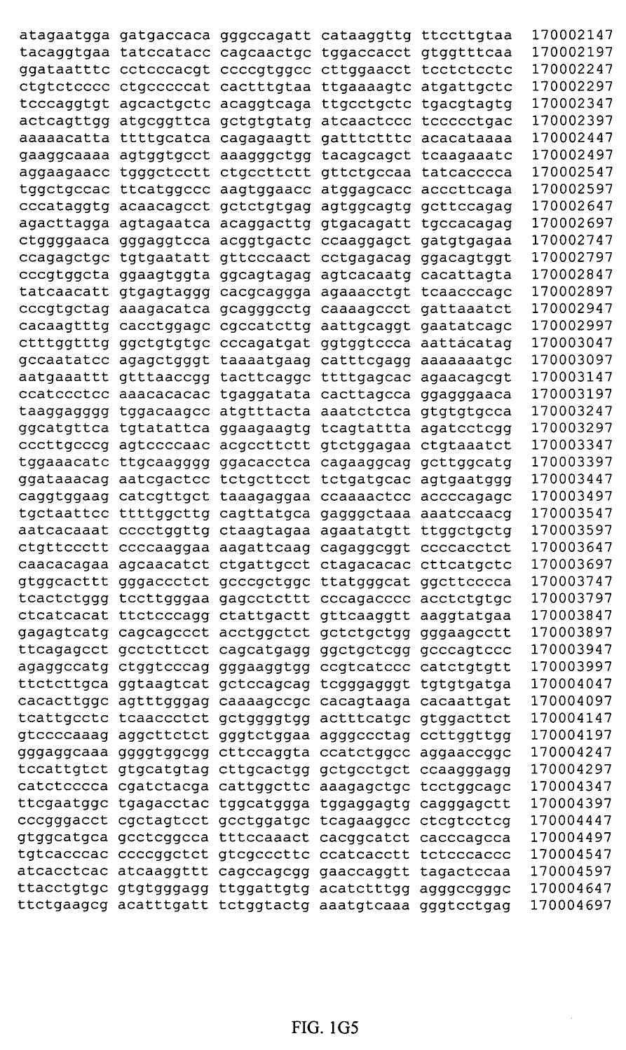

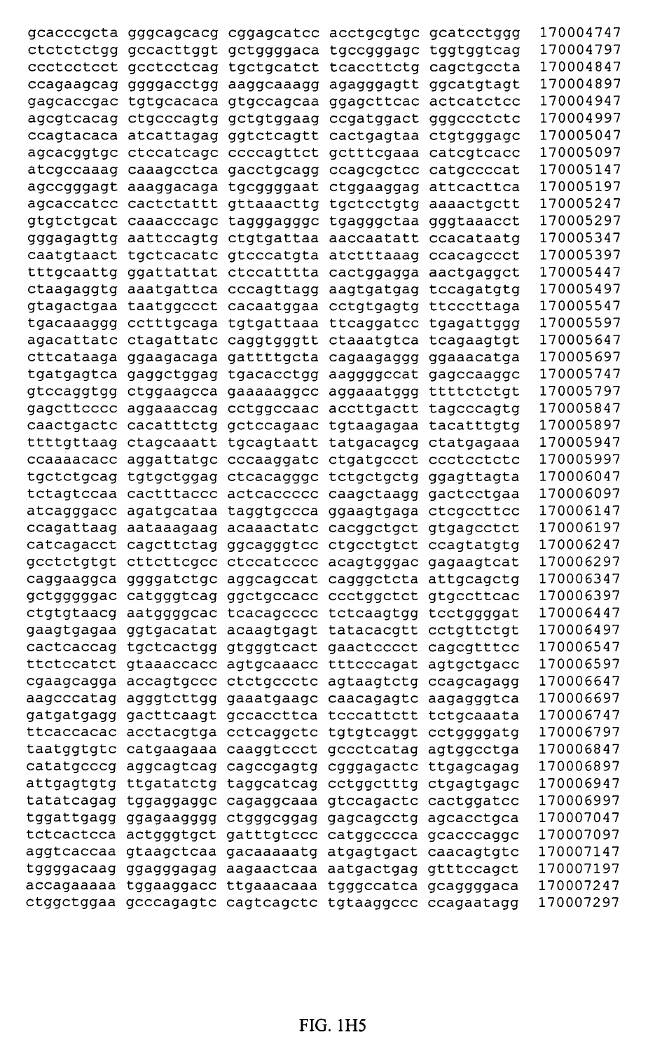

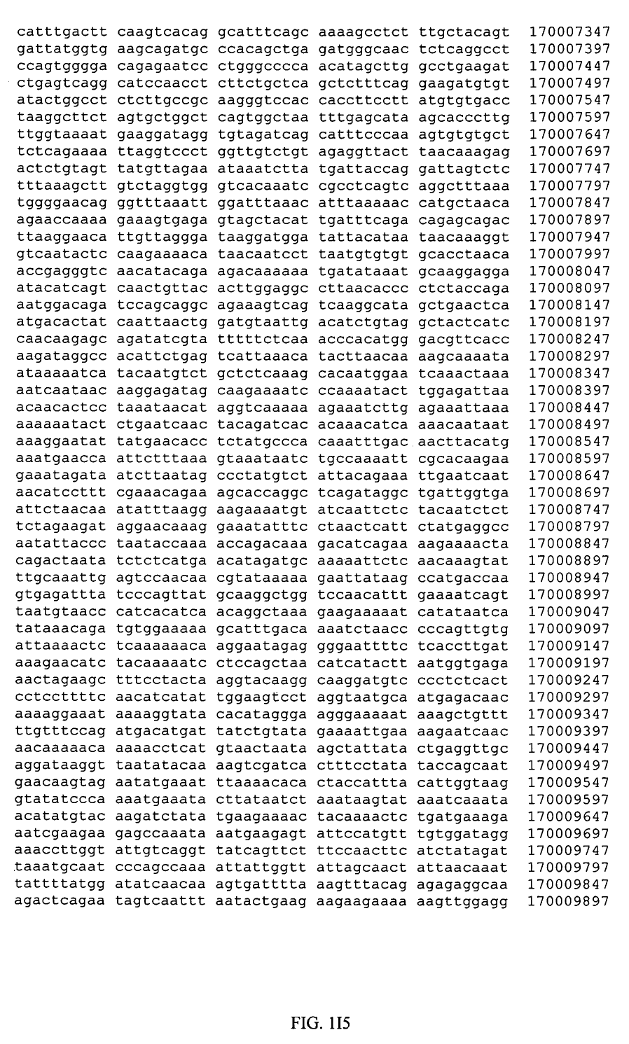

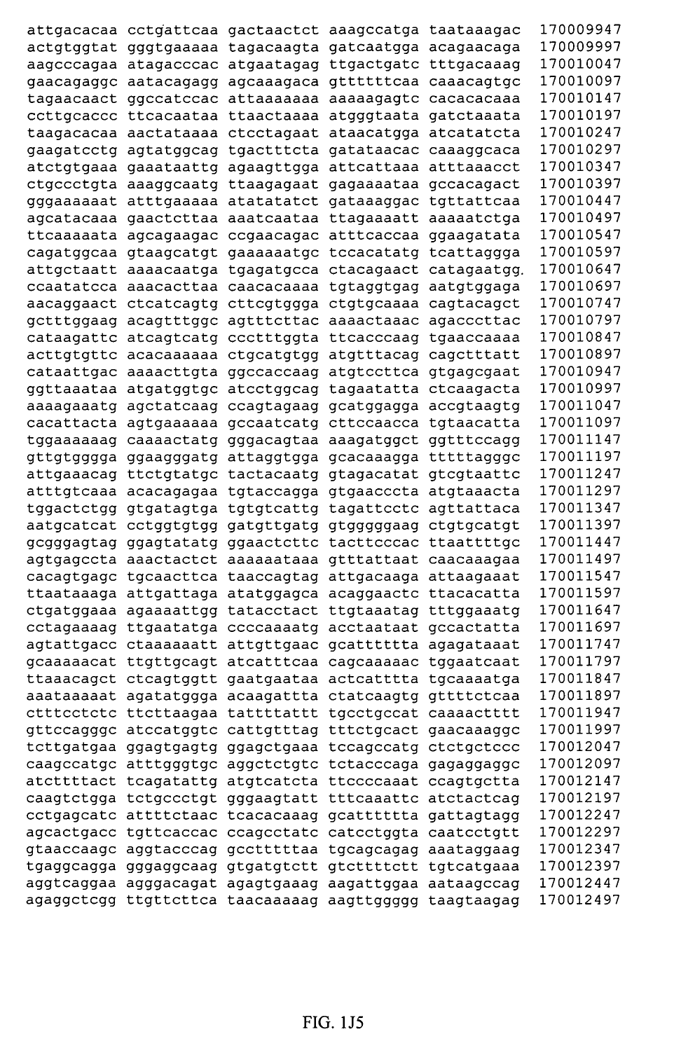

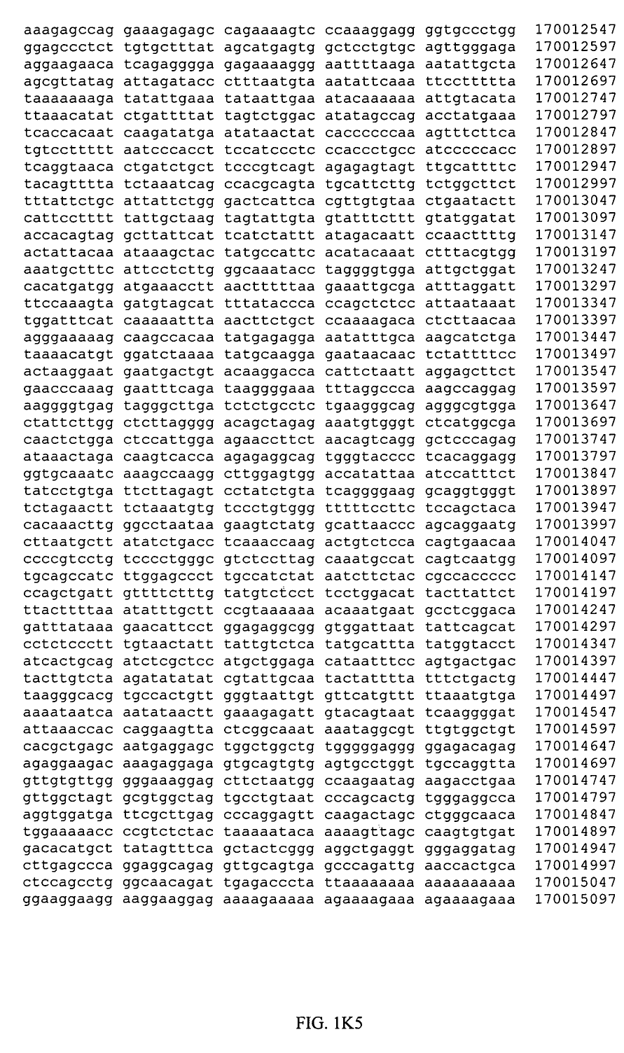

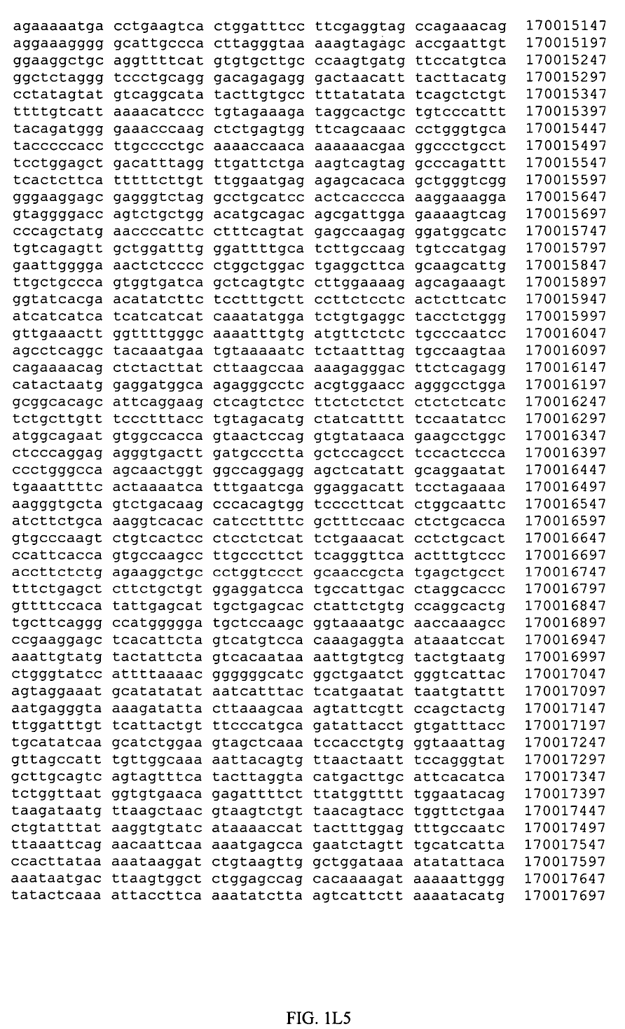

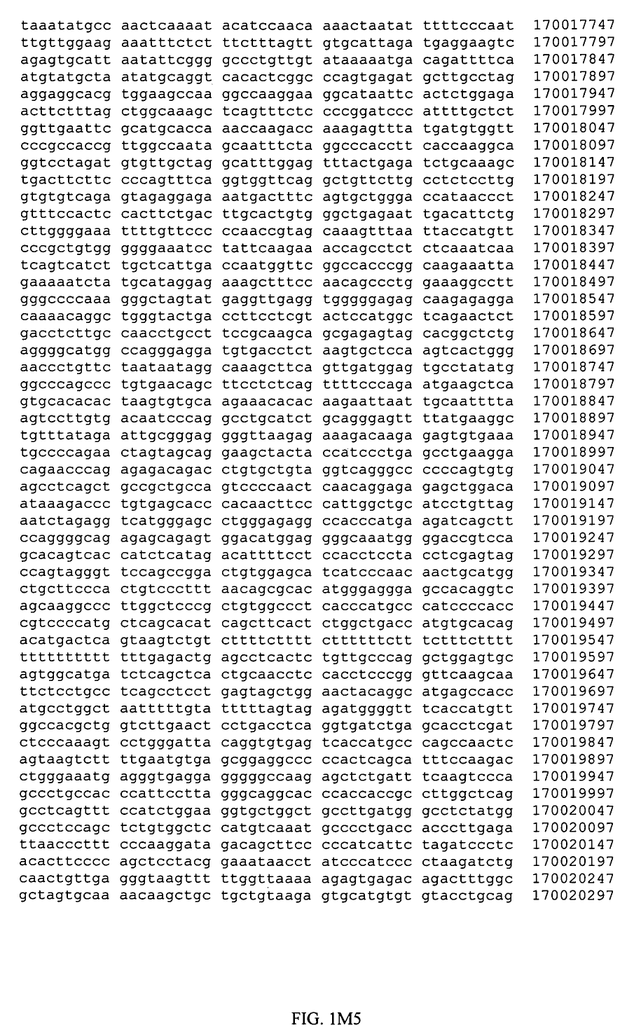

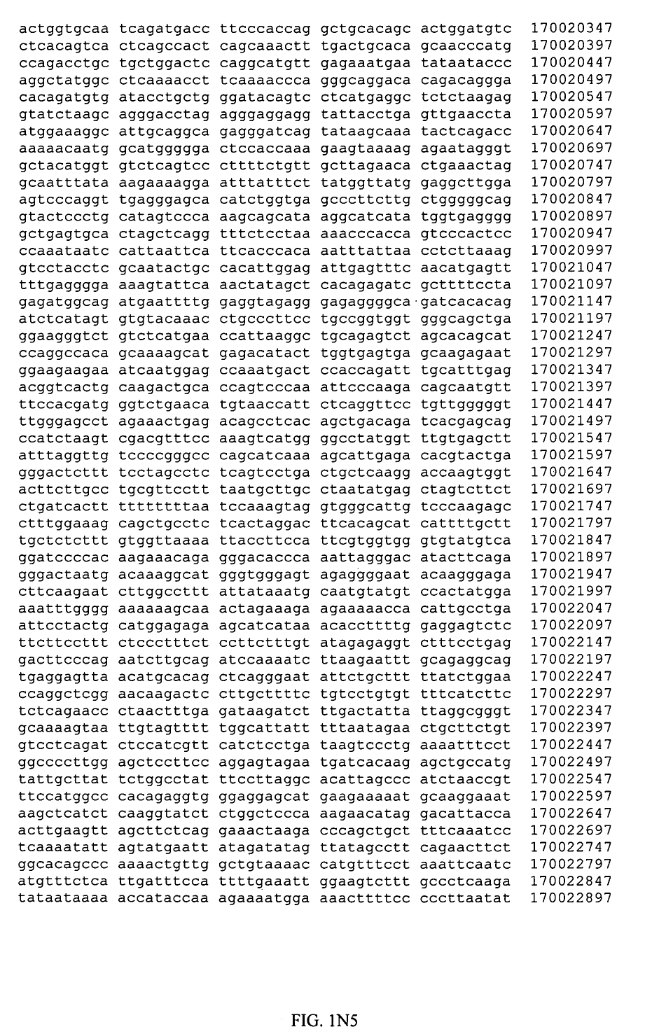

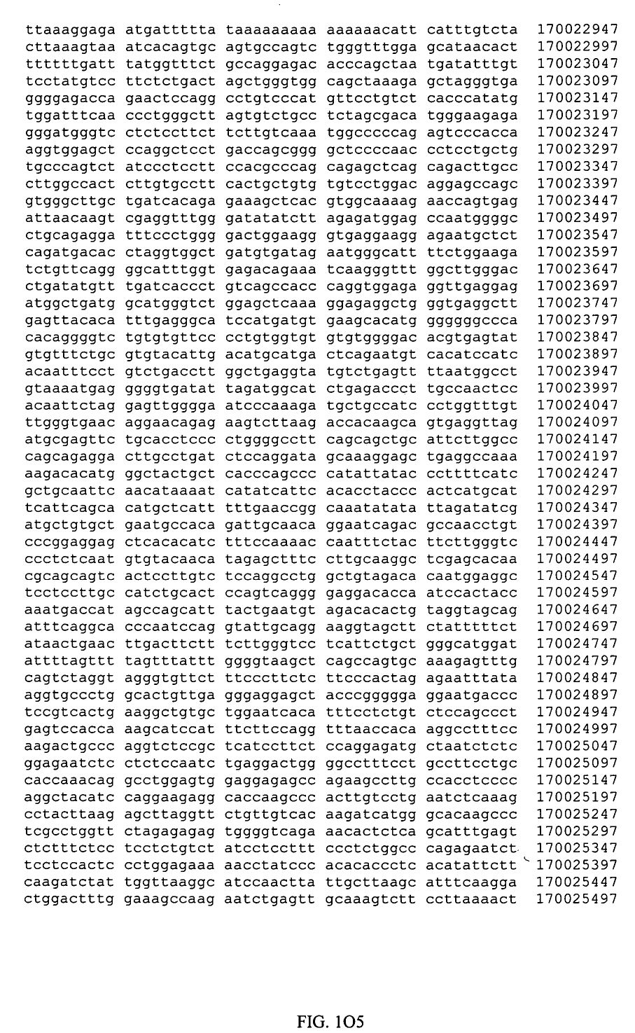

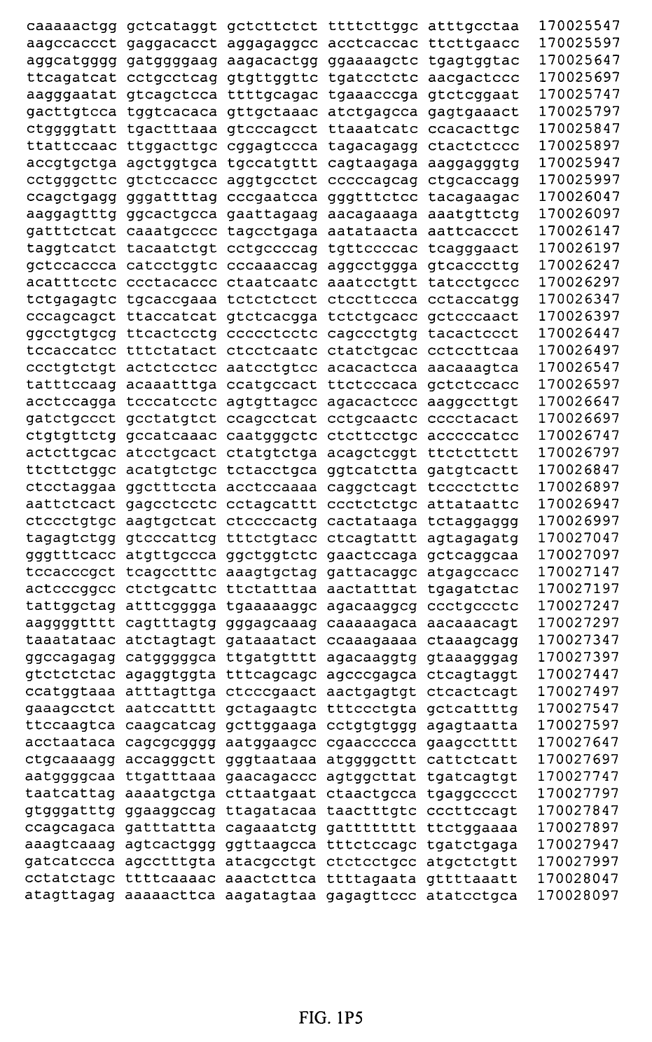

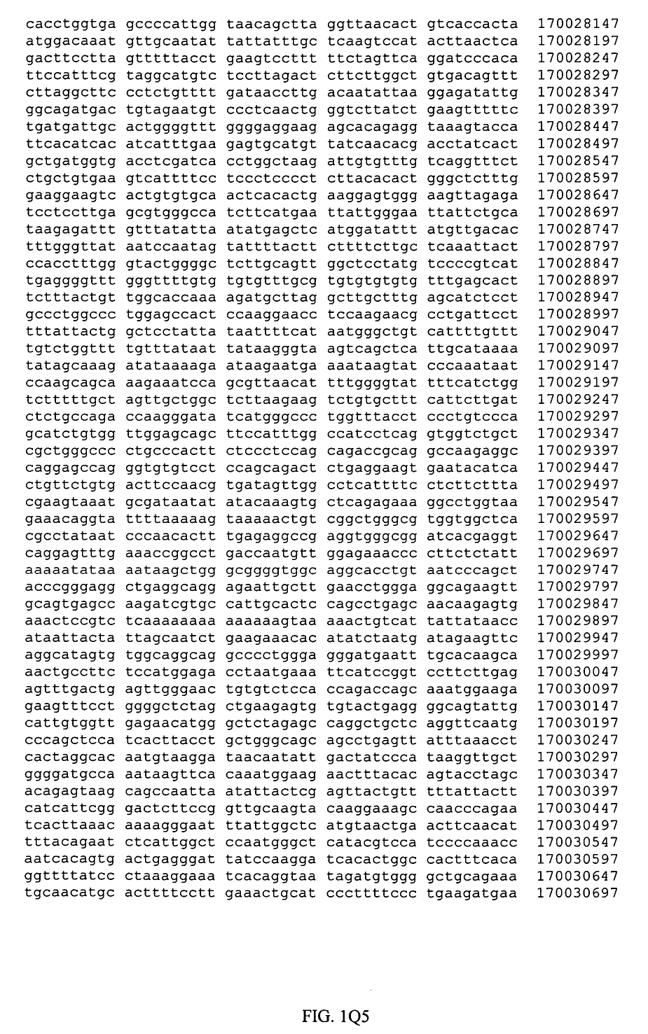

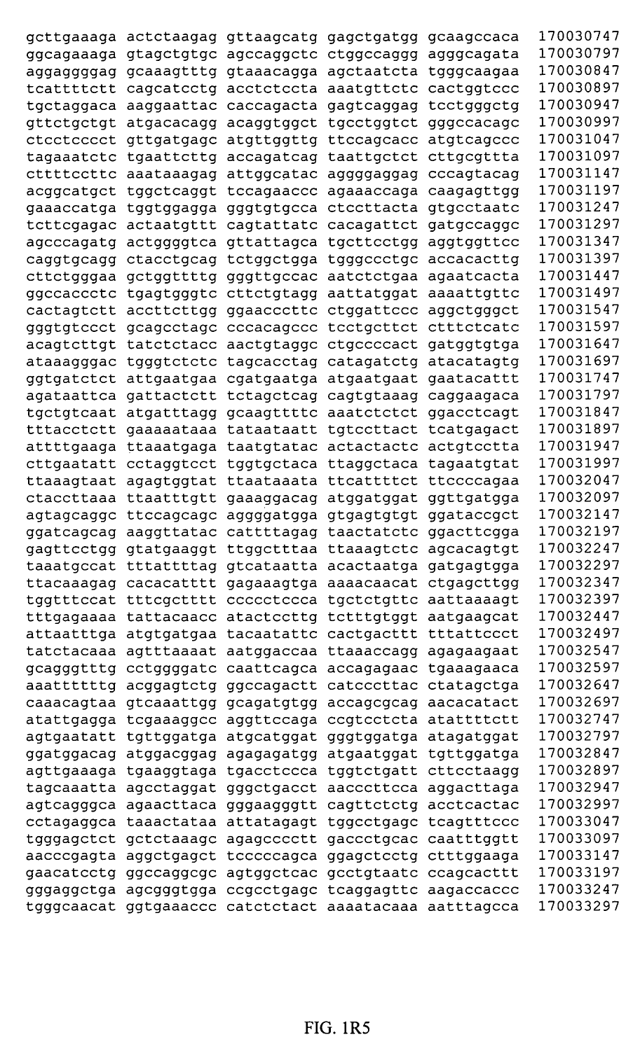

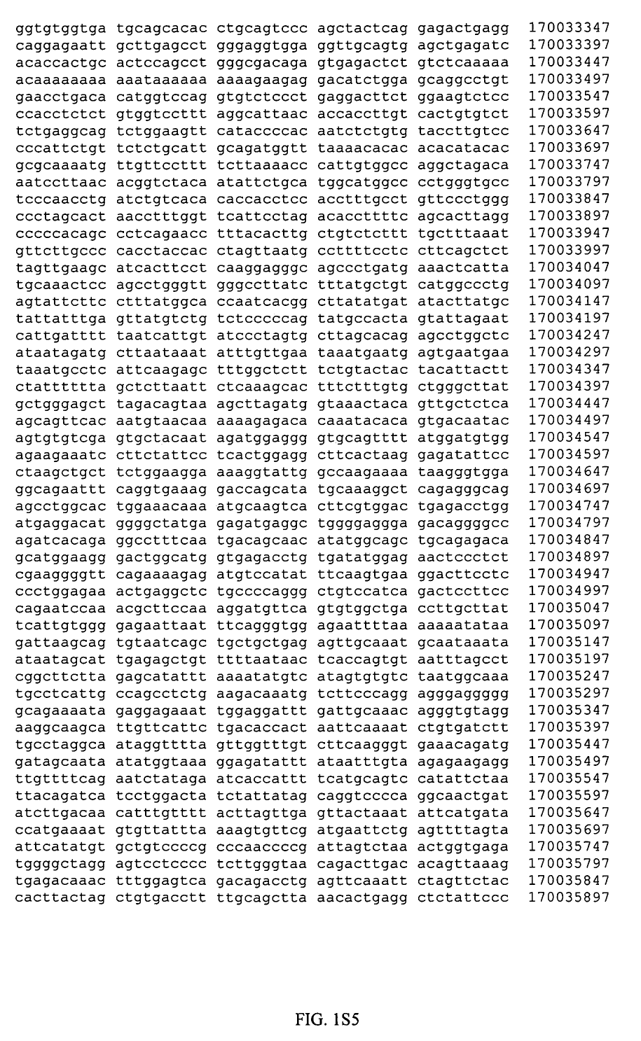

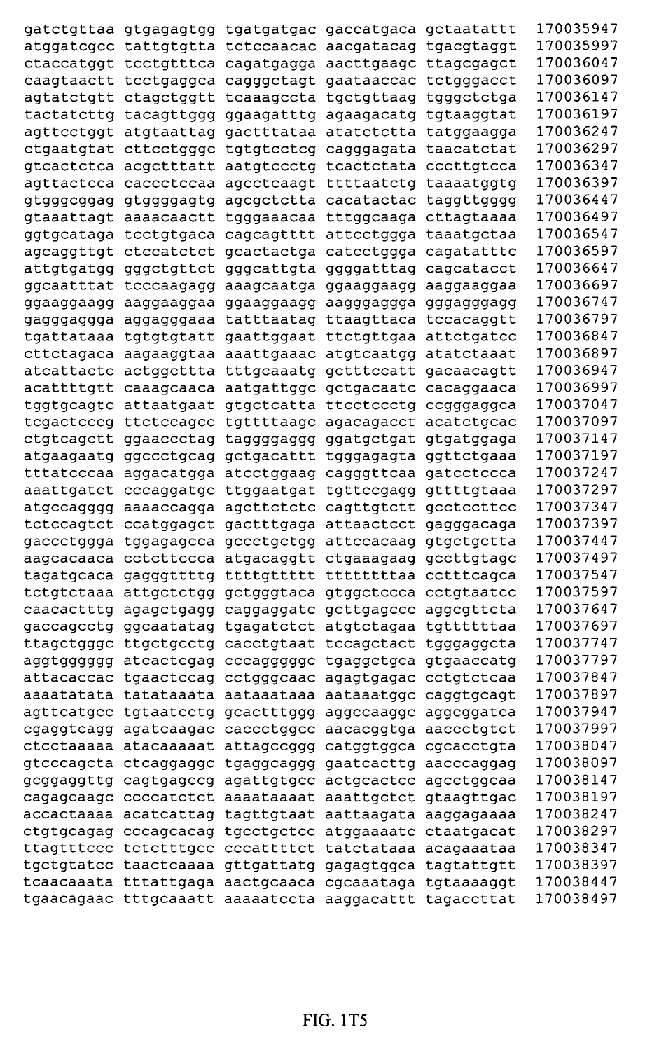

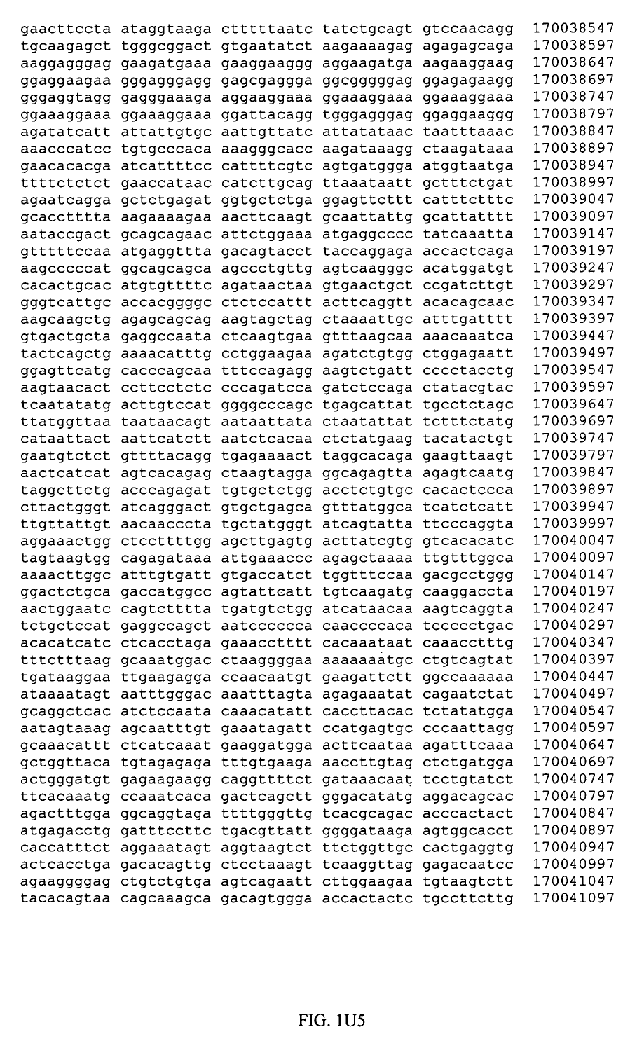

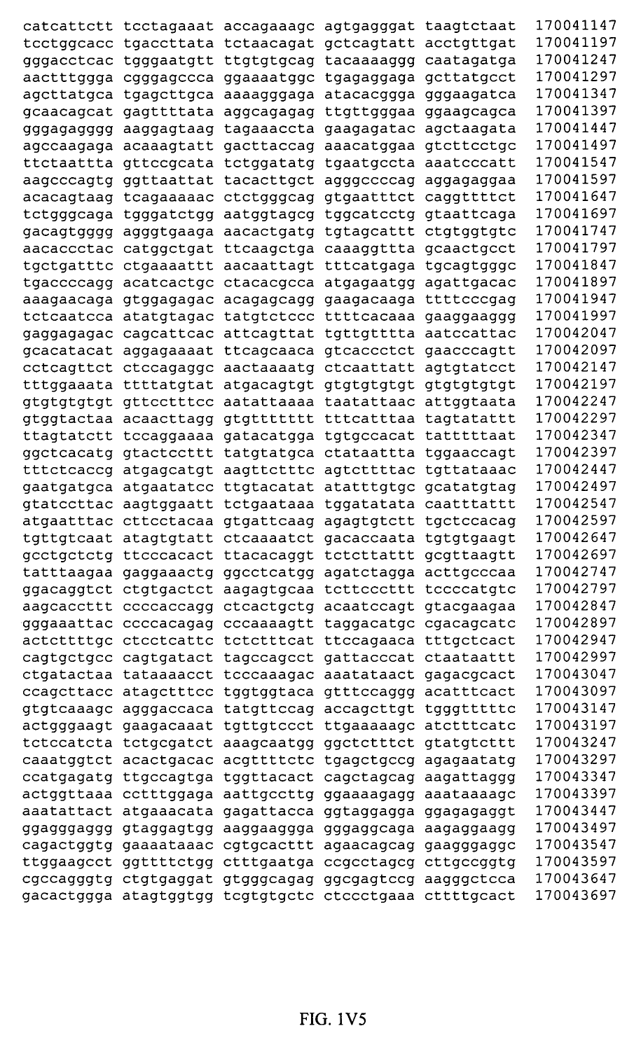

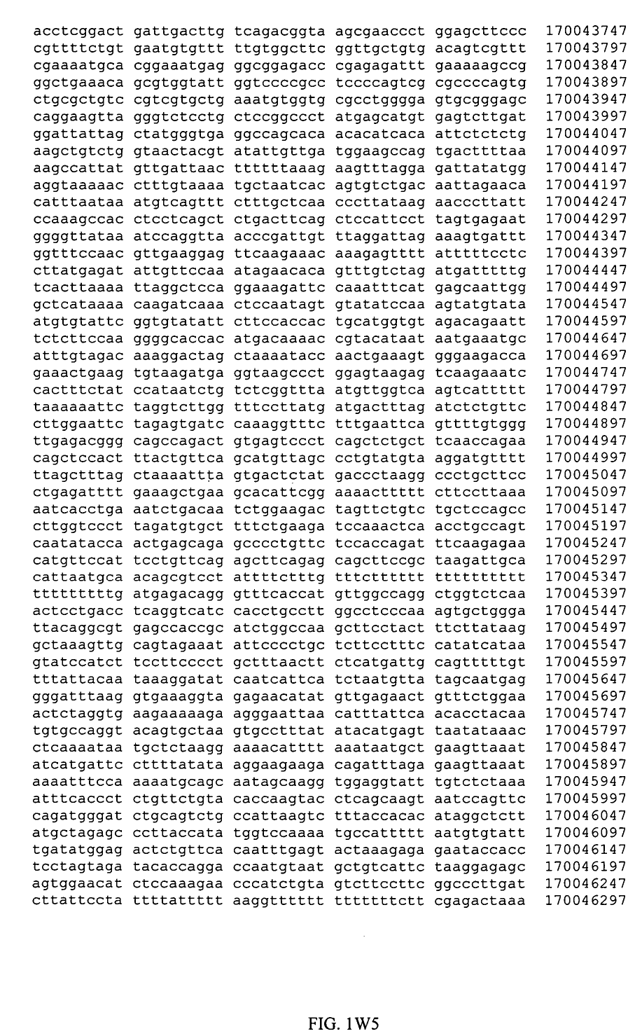

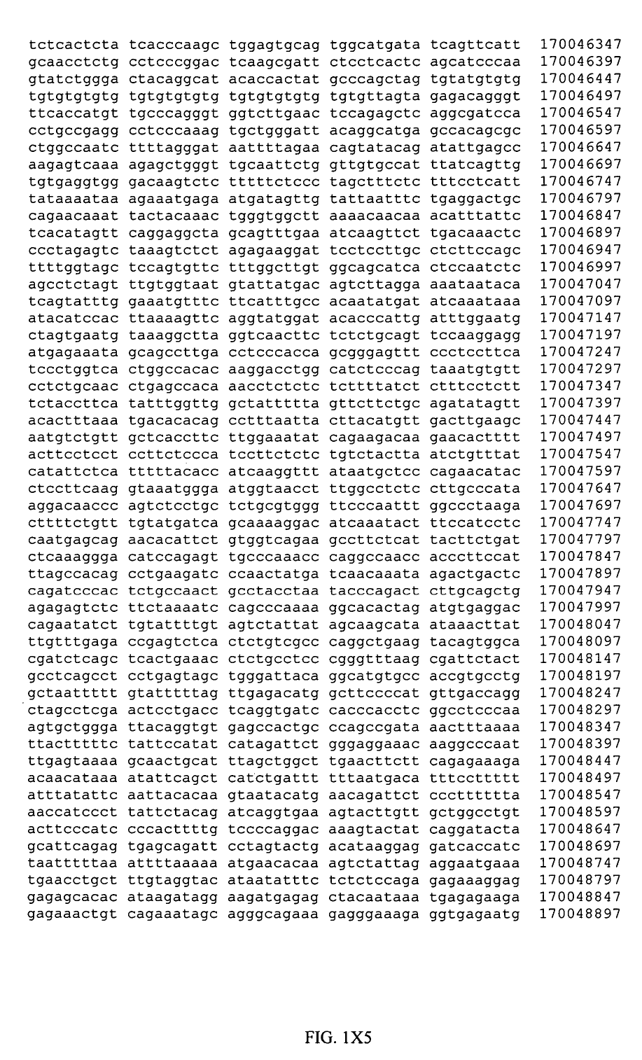

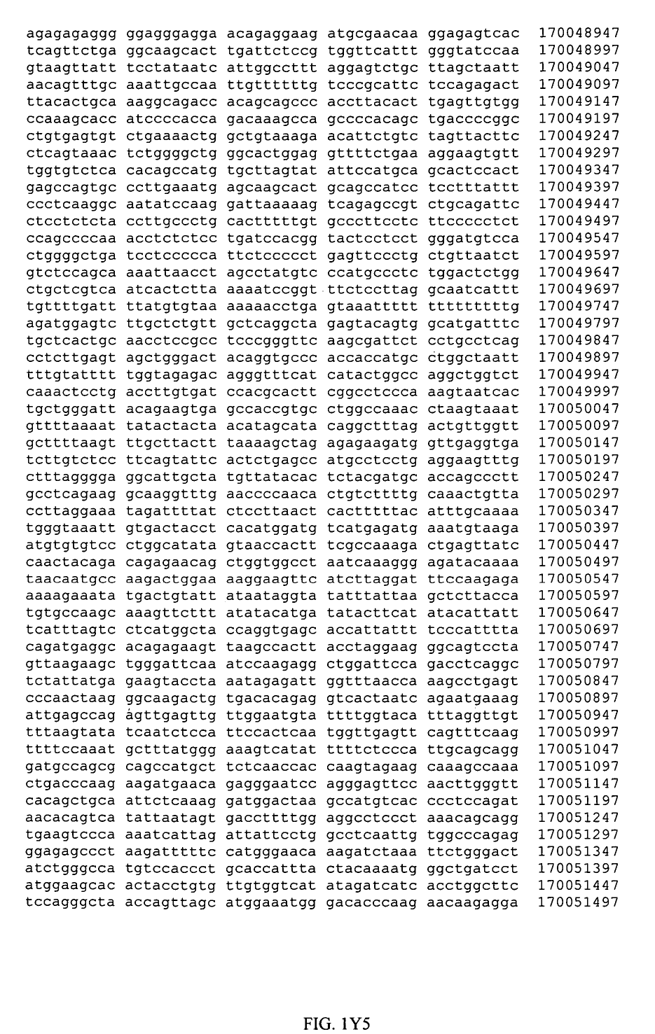

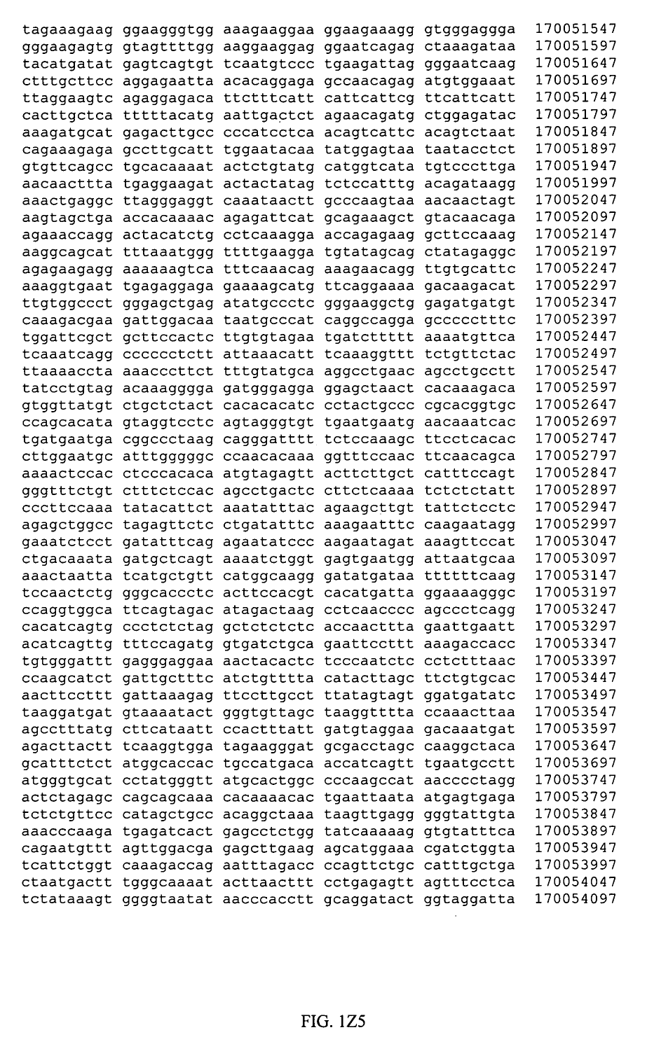

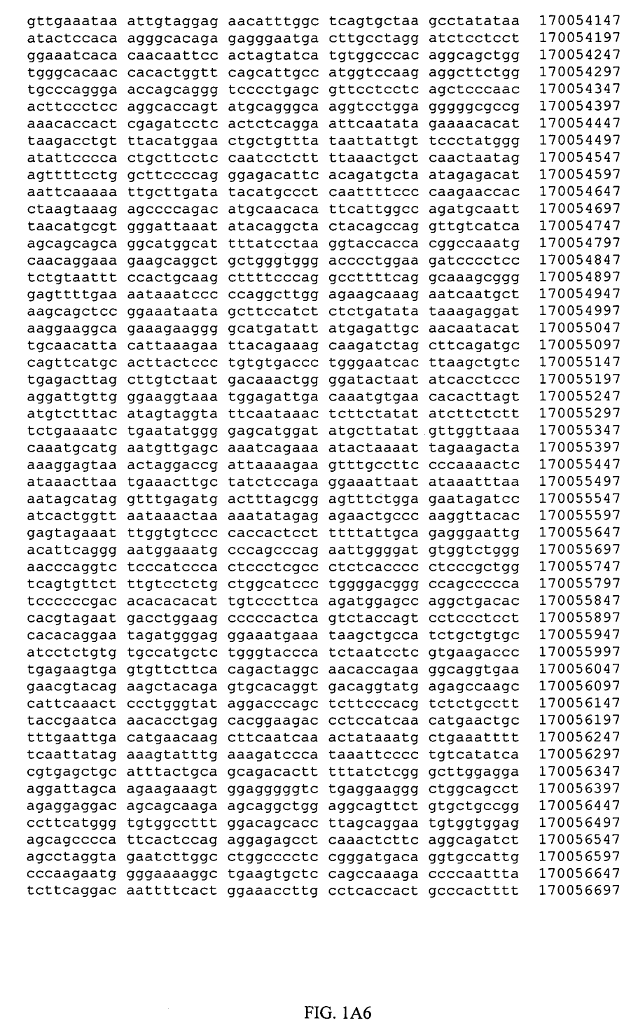

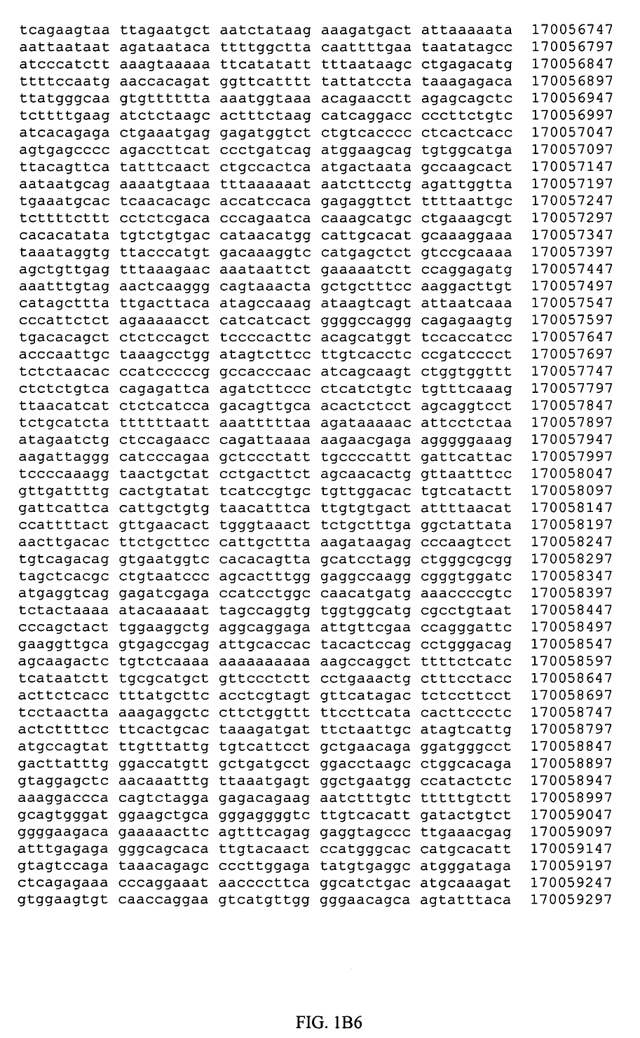

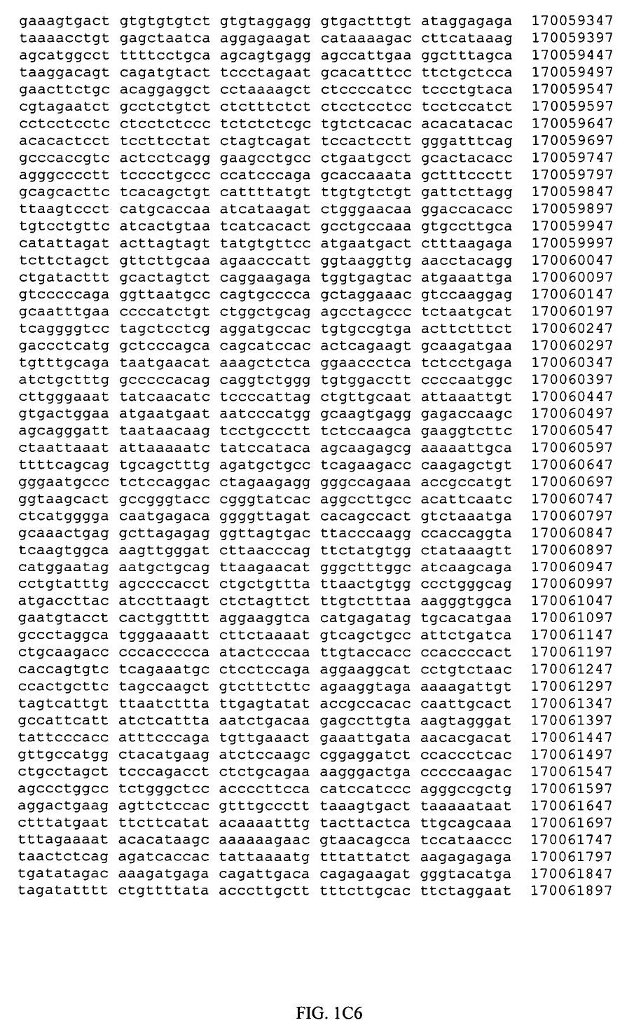

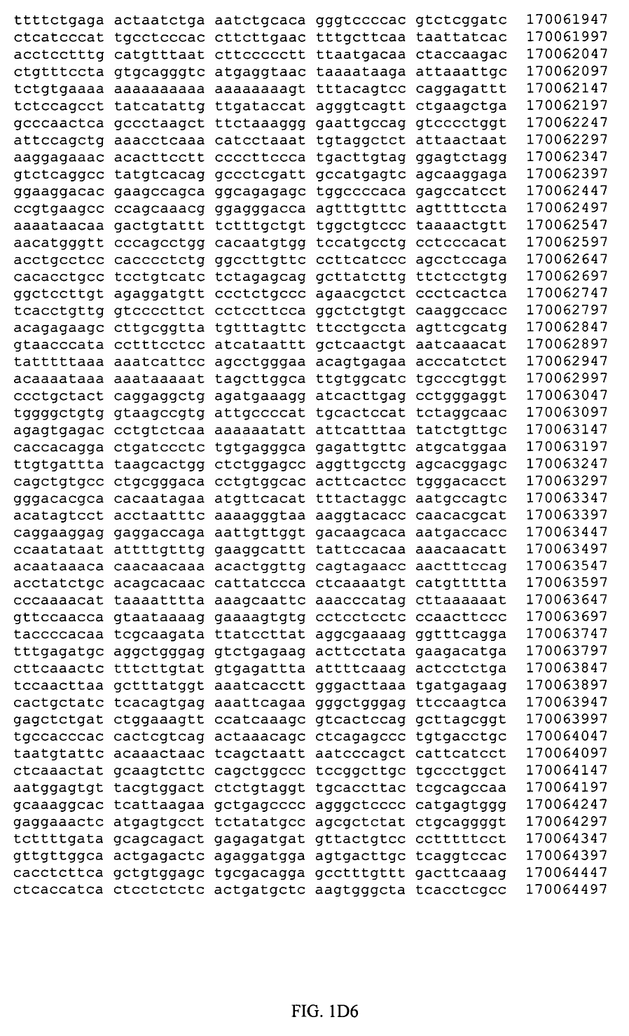

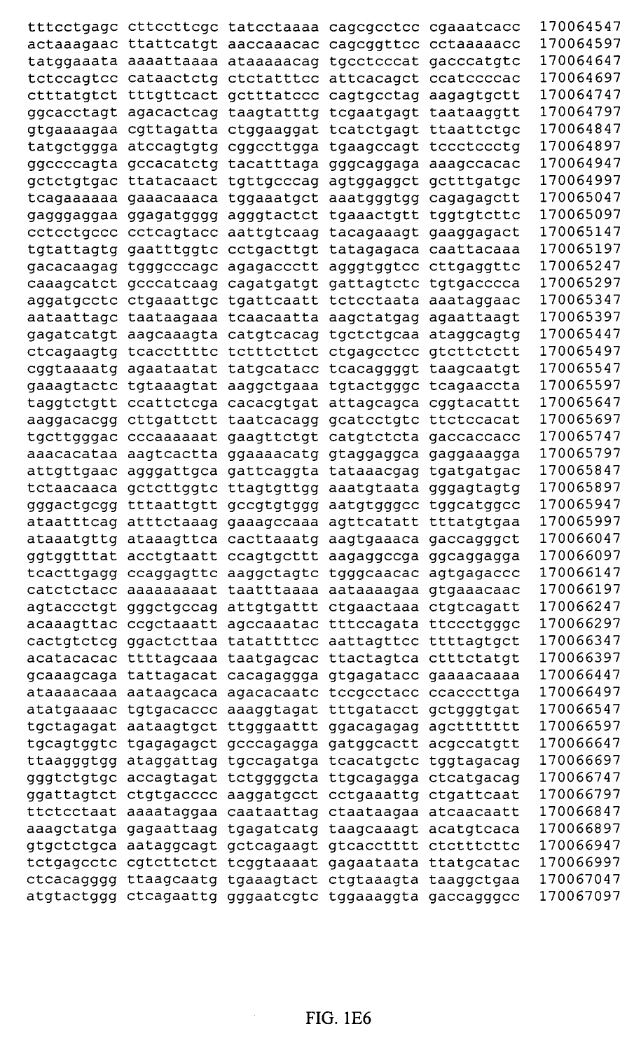

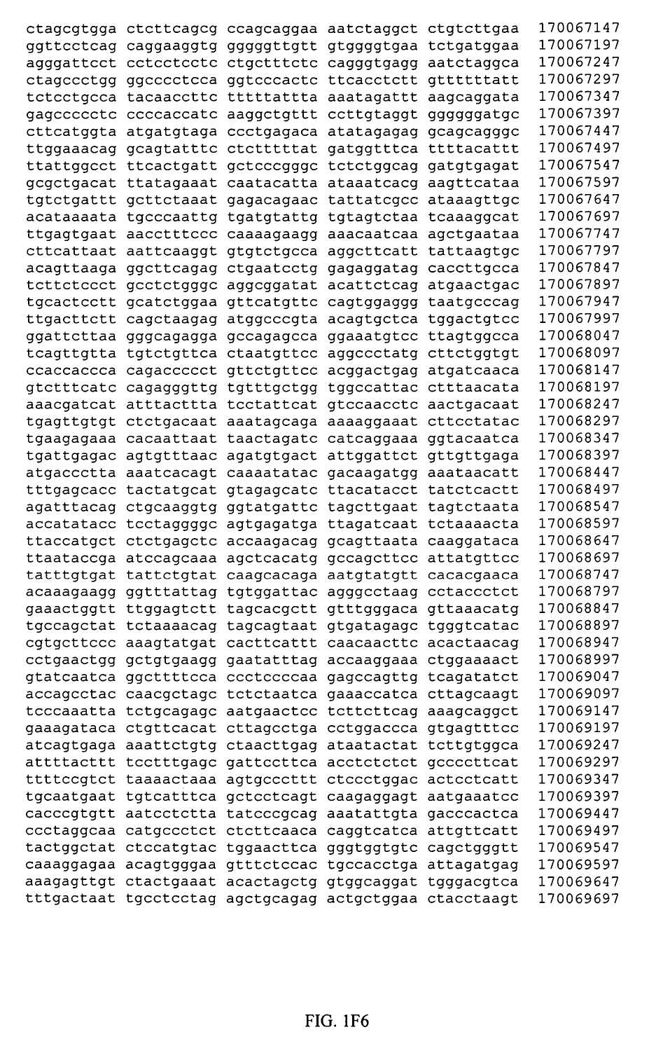

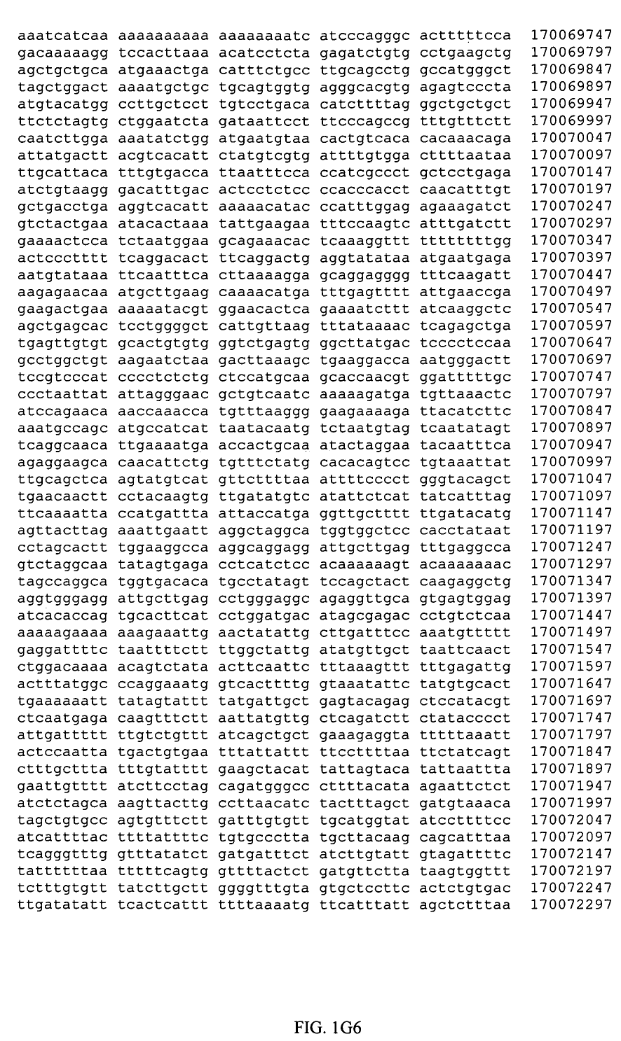

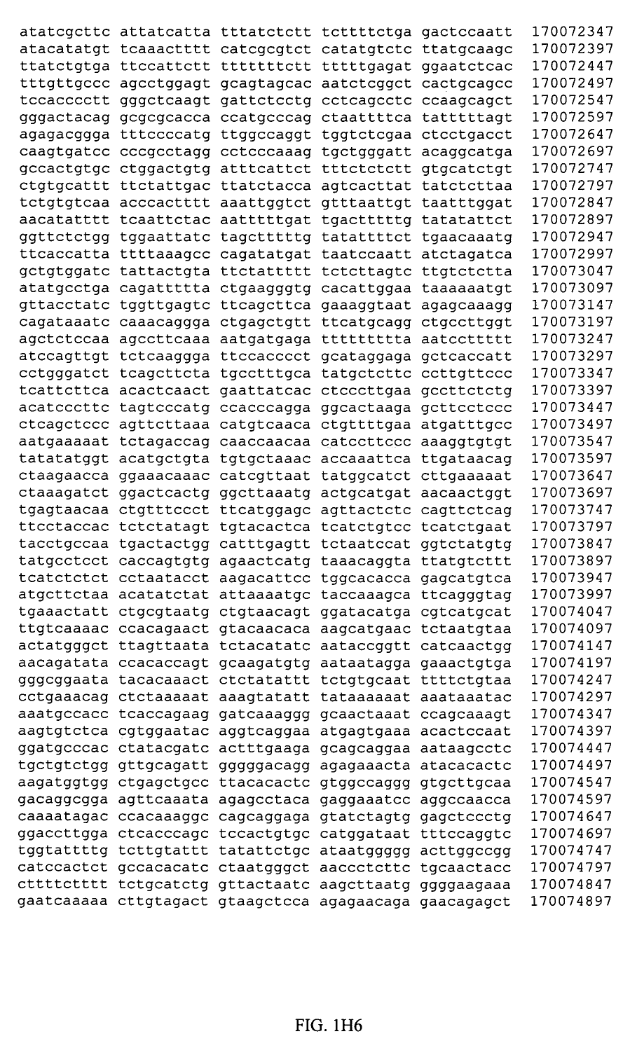

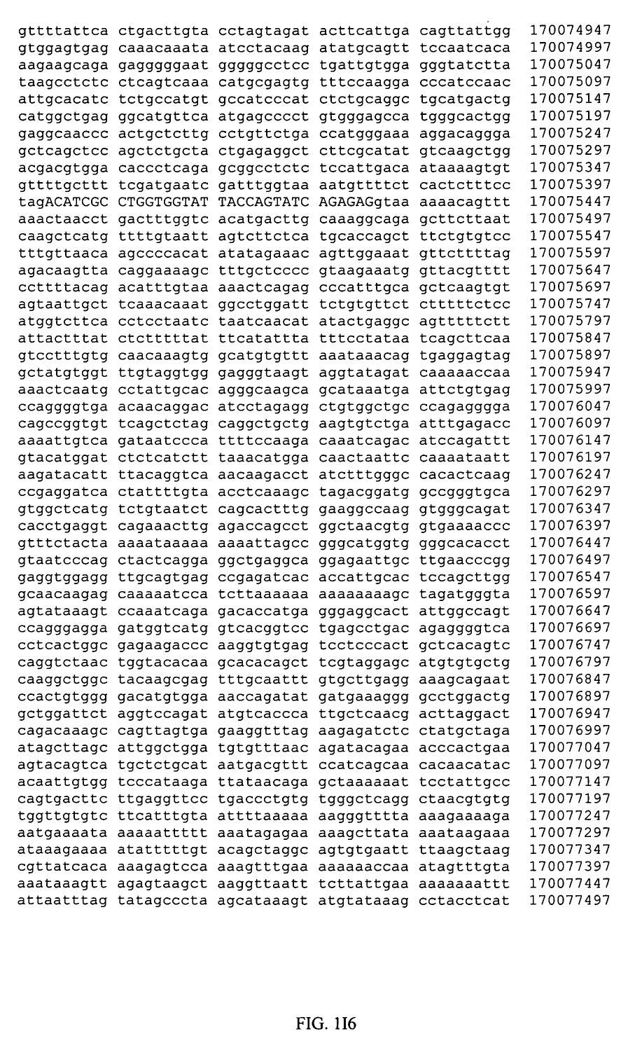

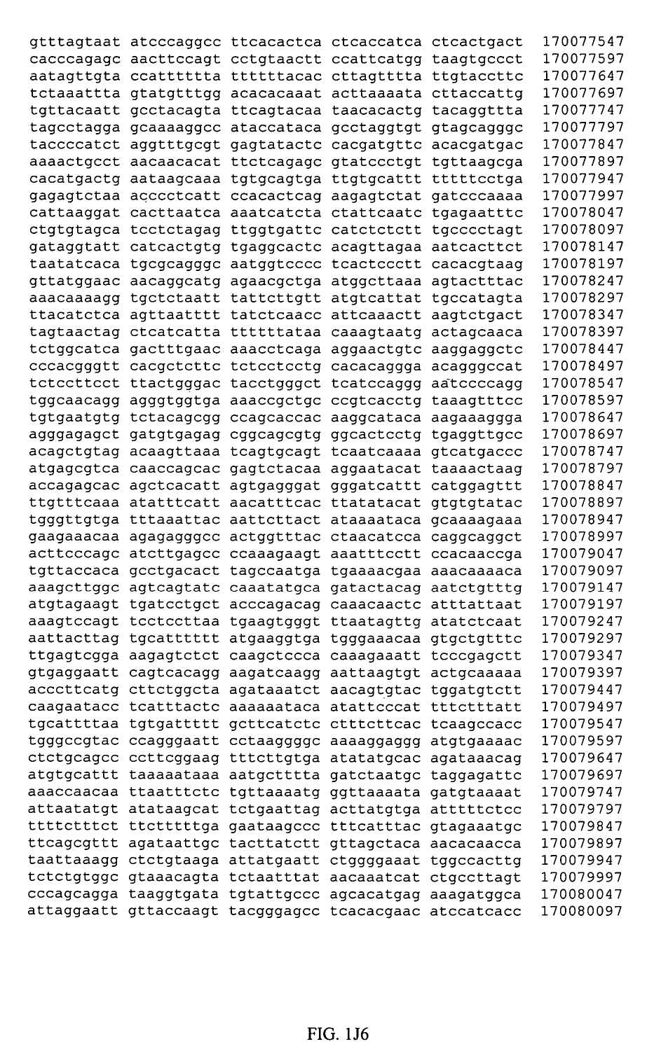

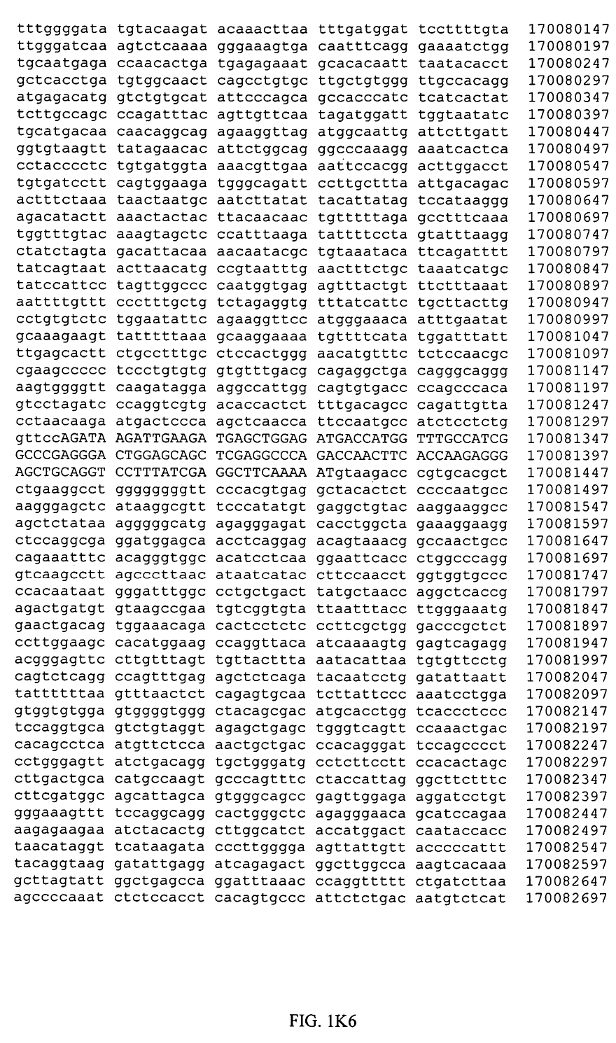

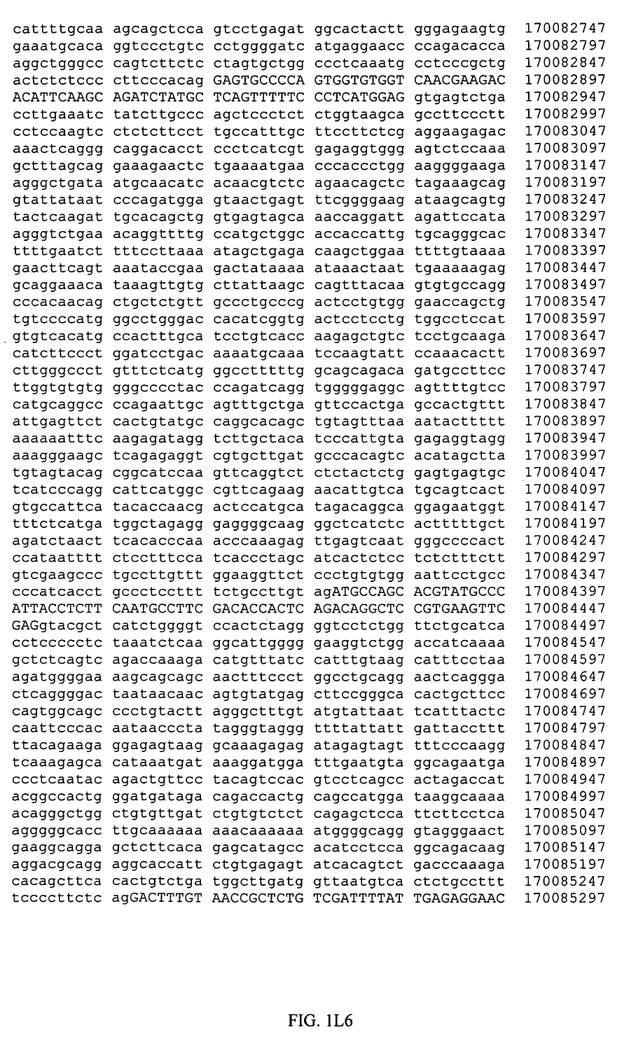

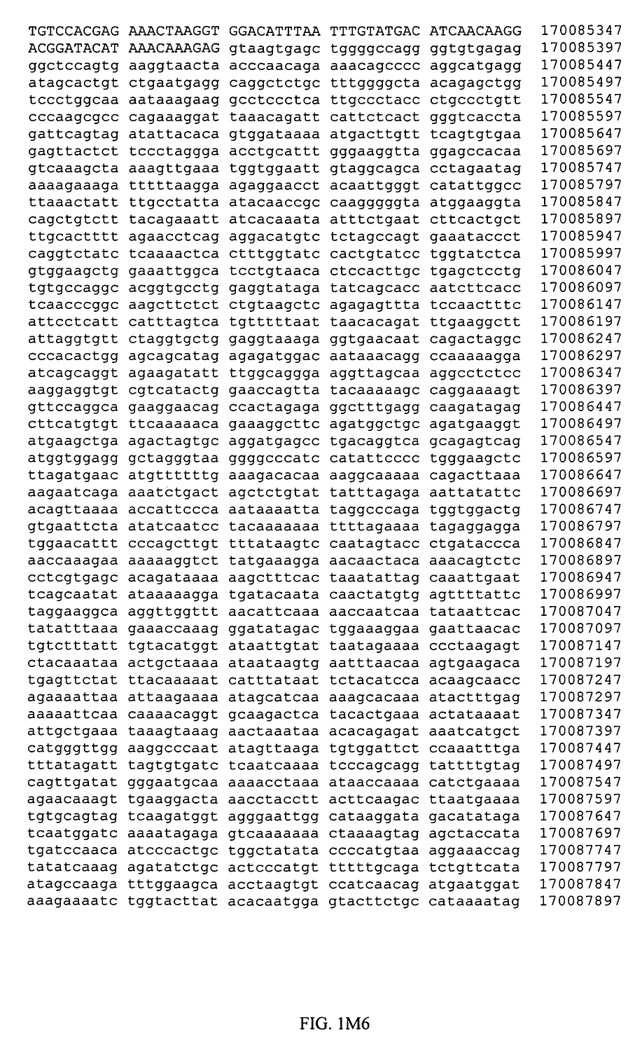

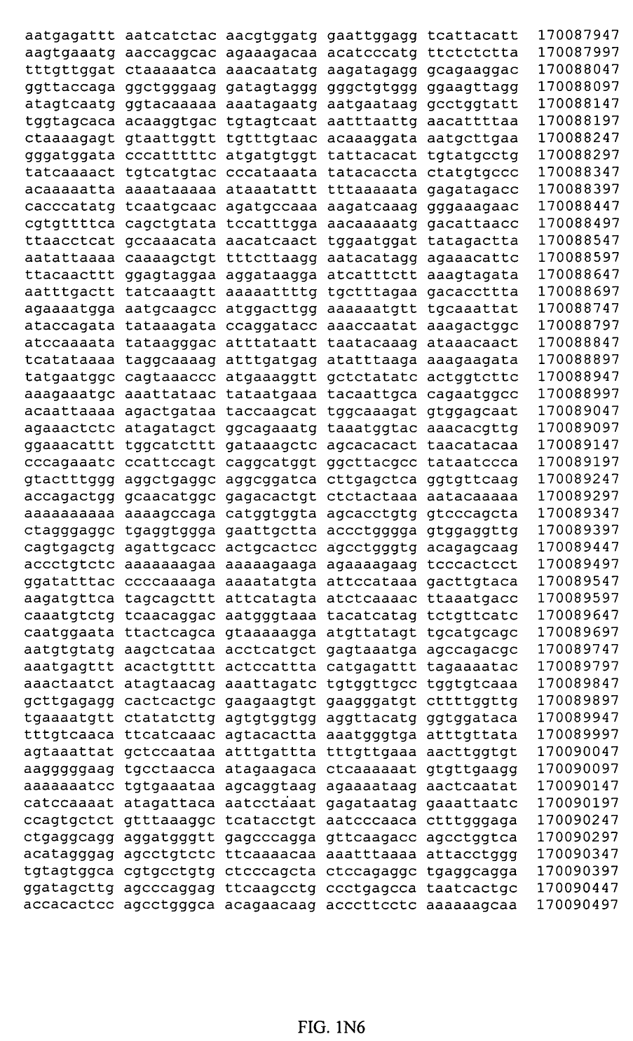

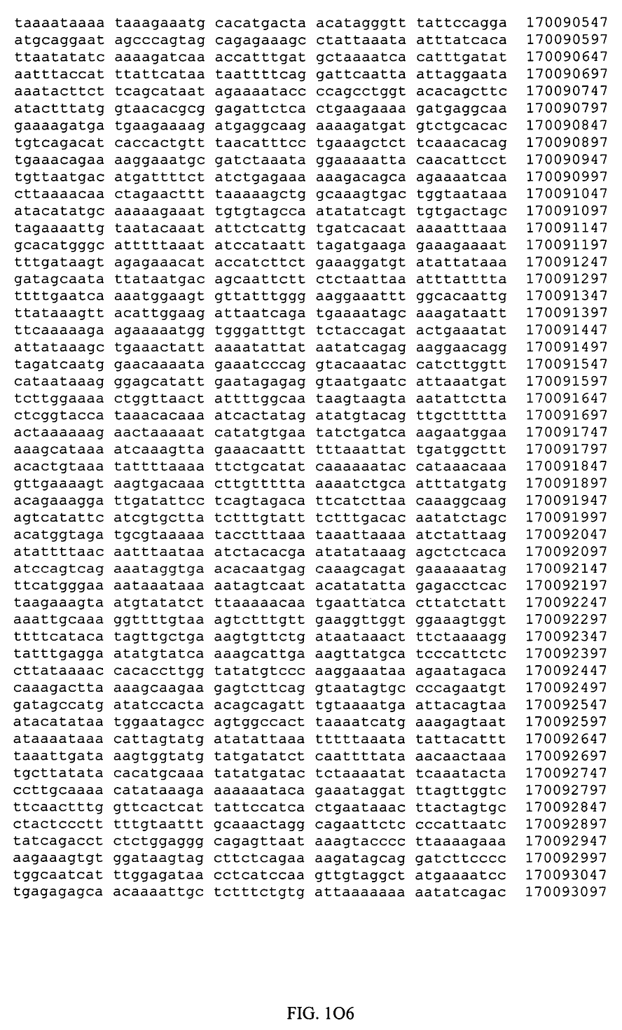

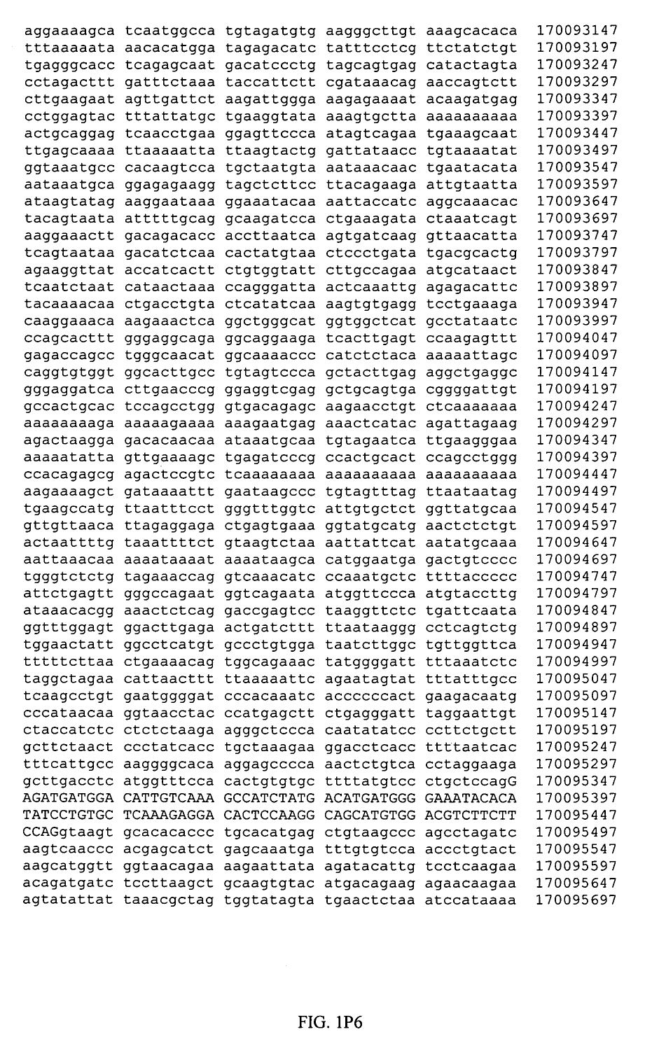

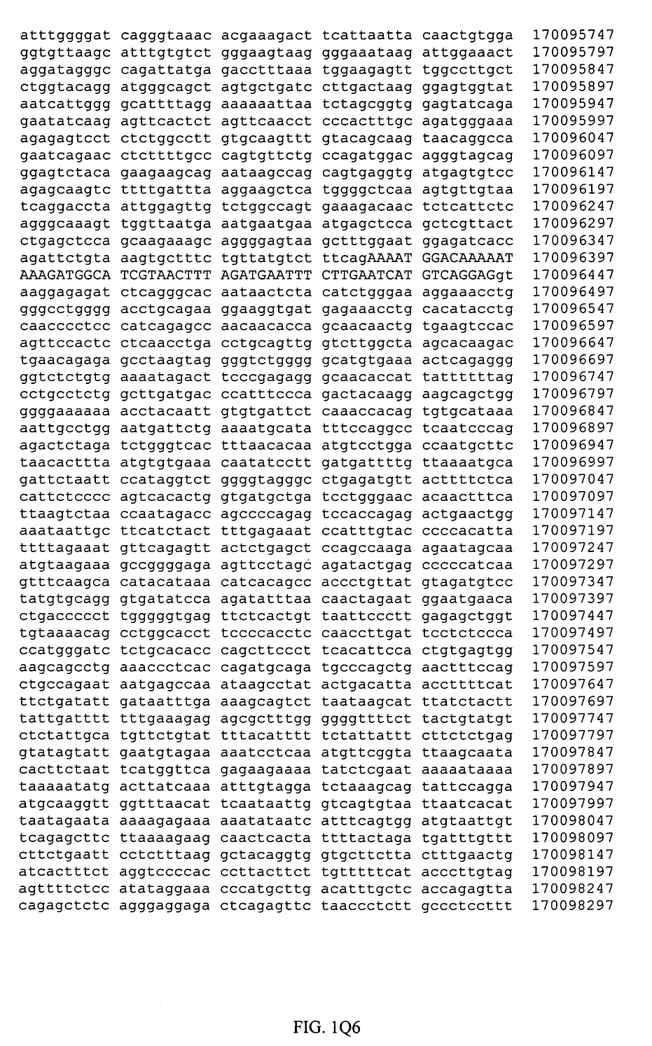

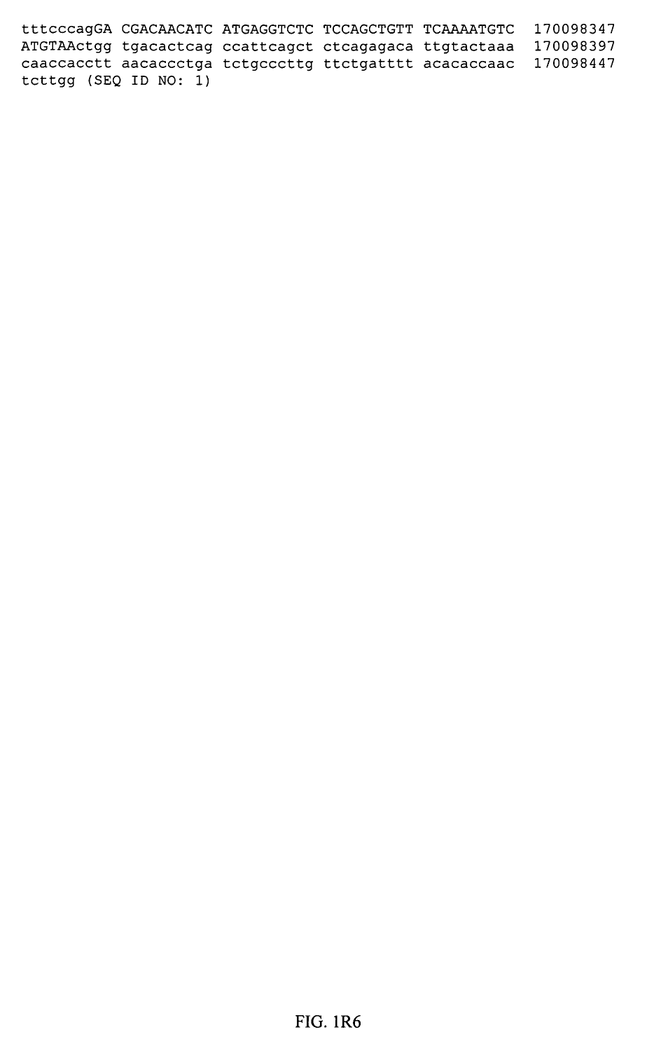

For example, the KChIP1 nucleic acid can the genomic sequence shown in FIG. 1, or a portion or fragment of the isolated nucleic acid molecule (e.g., cDNA or the gene) that encodes KChIP1 polypeptide. In certain embodiments, the isolated nucleic acid molecule comprises a nucleic acid molecule selected from the group consisting of SEQ ID NOs: 1 and 114-258 (e.g., in Table 10) or the complement of such a nucleic acid molecule.

Additionally, nucleic acid molecules of the invention can be fused to a marker sequence, for example, a sequence that encodes a polypeptide to assist in isolation or purification of the polypeptide. Such sequences include, but are not limited to, those that encode a glutathione-S-transferase (GST) fusion protein and those that encode a hemagglutinin A (HA) polypeptide marker from influenza.

An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention may be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material may be purified to essential homogeneity, for example as determined by PAGE or column chromatography such as HPLC. Preferably, an isolated nucleic acid molecule comprises at least about 50, 80 or 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 5 kb but not limited to 4 kb, 3 kb, 2 kb, 1 kb, 0.5 kb or 0.1 kb of nucleotides which flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells, as well as partially or substantially purified DNA molecules in solution. “Isolated” nucleic acid molecules also encompass in vivo and in vitro RNA transcripts of the DNA molecules of the present invention. An isolated nucleic acid molecule can include a nucleic acid molecule or nucleic acid sequence that is synthesized chemically or by recombinant means. Therefore, recombinant DNA contained in a vector is included in the definition of “isolated” as used herein. Also, isolated nucleic acid molecules include recombinant DNA molecules in heterologous organisms, as well as partially or substantially purified DNA molecules in solution. In vivo and in vitro RNA transcripts of the DNA molecules of the present invention are also encompassed by “isolated” nucleic acid sequences. Such isolated nucleic acid molecules are useful in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern blot analysis.

The present invention also pertains to nucleic acid molecules which are not necessarily found in nature but which encode a KChIP1 polypeptide, or another splicing variant of a KChIP1 polypeptide or polymorphic variant thereof. Thus, for example, the invention pertains to DNA molecules comprising a sequence that is different from the naturally occurring nucleotide sequence but which, due to the degeneracy of the genetic code, encode a KChIP1 polypeptide of the present invention. The invention also encompasses nucleic acid molecules encoding portions (fragments), or encoding variant polypeptides such as analogues or derivatives of a KChIP1 polypeptide. Such variants can be naturally occurring, such as in the case of allelic variation or single nucleotide polymorphisms, or non-naturally-occurring, such as those induced by various mutagens and mutagenic processes. Intended variations include, but are not limited to, addition, deletion and substitution of one or more nucleotides that can result in conservative or non-conservative amino acid changes, including additions and deletions. Preferably the nucleotide (and/or resultant amino acid) changes are silent or conserved; that is, they do not alter the characteristics or activity of a KChIP1 polypeptide. In one embodiment, the nucleic acid sequences are fragments that comprise one or more polymorphic microsatellite markers. In another embodiment, the nucleotide sequences are fragments that comprise one or more single nucleotide polymorphisms in a KChIP1 gene.

Other alterations of the nucleic acid molecules of the invention can include, for example, labeling, methylation, internucleotide modifications such as uncharged linkages (e.g., methyl phosphonates, phosphotriesters, phosphoamidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids). Also included are synthetic molecules that mimic nucleic acid molecules in the ability to bind to a designated sequence via hydrogen bonding and other chemical interactions. Such molecules include, for example, those in which peptide linkages substitute for phosphate linkages in the backbone of the molecule.

The invention also pertains to nucleic acid molecules that hybridize under high stringency hybridization conditions, such as for selective hybridization, to a nucleotide sequence described herein (e.g., nucleic acid molecules which specifically hybridize to a nucleotide sequence encoding polypeptides described herein, and, optionally, have an activity of the polypeptide). In one embodiment, the invention includes variants described herein which hybridize under high stringency hybridization conditions (e.g., for selective hybridization) to a nucleotide sequence comprising a nucleotide sequence selected from the group consisting of SEQ ID NOs: 114-258. In another embodiment, the invention includes variants described herein that hybridize under high stringency hybridization conditions (e.g., for selective hybridization) to a nucleotide sequence encoding an amino acid sequence or a polymorphic variant thereof. In another embodiment, the variant that hybridizes under high stringency hybridizations has an activity of a KChIP1 polypeptide.

Such nucleic acid molecules can be detected and/or isolated by specific hybridization (e.g., under high stringency conditions). “Specific hybridization,” as used herein, refers to the ability of a first nucleic acid to hybridize to a second nucleic acid in a manner such that the first nucleic acid does not hybridize to any nucleic acid other than to the second nucleic acid (e.g., when the first nucleic acid has a higher similarity to the second nucleic acid than to any other nucleic acid in a sample wherein the hybridization is to be performed). “Stringency conditions” for hybridization is a term of art which refers to the incubation and wash conditions, e.g., conditions of temperature and buffer concentration, which permit hybridization of a particular nucleic acid to a second nucleic acid; the first nucleic acid may be perfectly (i.e., 100%) complementary to the second, or the first and second may share some degree of complementarity which is less than perfect (e.g., 70%, 75%, 85%, 90%, 95%). For example, certain high stringency conditions can be used which distinguish perfectly complementary nucleic acids from those of less complementarity. “High stringency conditions”, “moderate stringency conditions” and “low stringency conditions” for nucleic acid hybridizations are explained on pages 2.10.1-2.10.16 and pages 6.3.1-6.3.6 in Current Protocols in Molecular Biology (Ausubel, F. M. et al., “Current Protocols in Molecular Biology”, John Wiley & Sons, (2001)), the entire teachings of which are incorporated by reference herein). The exact conditions which determine the stringency of hybridization depend not only on ionic strength (e.g., 0.2×SSC, 0.1×SSC), temperature (e.g., room temperature, 42° C., 68° C.) and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, equivalent conditions can be determined by varying one or more of these parameters while maintaining a similar degree of identity or similarity between the two nucleic acid molecules. Typically, conditions are used such that sequences at least about 60%, at least about 70%, at least about 80%, at least about 90%, or at least about 95% or more identical to each other remain hybridized to one another. By varying hybridization conditions from a level of stringency at which no hybridization occurs to a level at which hybridization is first observed, conditions which will allow a given sequence to hybridize (e.g., selectively) with the most similar sequences in the sample can be determined.

Exemplary conditions are described in Krause, M. H. and S. A. Aaronson, Methods in Enzymology 200:546-556 (1991), and in, Ausubel, et al., “Current Protocols in Molecular Biology”, John Wiley & Sons, (2001), which describes the determination of washing conditions for moderate or low stringency conditions. Washing is the step in which conditions are usually set so as to determine a minimum level of complementarity of the hybrids. Generally, starting from the lowest temperature at which only homologous hybridization occurs, each ° C. by which the final wash temperature is reduced (holding SSC concentration constant) allows an increase by 1% in the maximum extent of mismatching among the sequences that hybridize. Generally, doubling the concentration of SSC results in an increase in Tₘ of −17° C. Using these guidelines, the washing temperature can be determined empirically for high, moderate or low stringency, depending on the level of mismatch sought.

For example, a low stringency wash can comprise washing in a solution containing 0.2×SSC/0.1% SDS for 10 minutes at room temperature; a moderate stringency wash can comprise washing in a pre-warmed solution (42° C.) solution containing 0.2×SSC/0.1% SDS for 15 minutes at 42° C.; and a high stringency wash can comprise washing in pre-warmed (68° C.) solution containing 0.1×SSC/0.1% SDS for 15 minutes at 68° C. Furthermore, washes can be performed repeatedly or sequentially to obtain a desired result as known in the art. Equivalent conditions can be determined by varying one or more of the parameters given as an example, as known in the art, while maintaining a similar degree of identity or similarity between the target nucleic acid molecule and the primer or probe used.

The percent homology or identity of two nucleotide or amino acid sequences can be determined by aligning the sequences for optimal comparison purposes (e.g., gaps can be introduced in the sequence of a first sequence for optimal alignment). The nucleotides or amino acids at corresponding positions are then compared, and the percent identity between the two sequences is a function of the number of identical positions shared by the sequences (i.e., % identity=# of identical positions/total # of positions×100). When a position in one sequence is occupied by the same nucleotide or amino acid residue as the corresponding position in the other sequence, then the molecules are homologous at that position. As used herein, nucleic acid or amino acid “homology” is equivalent to nucleic acid or amino acid “identity”. In certain embodiments, the length of a sequence aligned for comparison purposes is at least 30%, for example, at least 40%, in certain embodiments at least 60%, and in other embodiments at least 70%, 80%, 90% or 95% of the length of the reference sequence. The actual comparison of the two sequences can be accomplished by well-known methods, for example, using a mathematical algorithm. A preferred, non-limiting example of such a mathematical algorithm is described in Karlin et al., Proc. Natl. Acad. Sci. USA 90:5873-5877 (1993). Such an algorithm is incorporated into the NBLAST and XBLAST programs (version 2.0) as described in Altschul et al., Nucleic Acids Res. 25:389-3402 (1997). When utilizing BLAST and Gapped BLAST programs, the default parameters of the respective programs (e.g., NBLAST) can be used. In one embodiment, parameters for sequence comparison can be set at score=100, wordlength=12, or can be varied (e.g., W=5 or W=20).

Another preferred, non-limiting example of a mathematical algorithm utilized for the comparison of sequences is the algorithm of Myers and Miller, CABIOS 4(1): 11-17 (1988). Such an algorithm is incorporated into the ALIGN program (version 2.0) which is part of the GCG sequence alignment software package (Accelrys, Cambridge, UK). When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, Comput. Appl. Biosci. 10:3-5 (1994); and FASTA described in Pearson and Lipman, Proc. Natl. Acad. Sci. USA 85:2444-8 (1988).

In another embodiment, the percent identity between two amino acid sequences can be accomplished using the GAP program in the GCG software package using either a BLOSUM63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the GCG software package using a gap weight of 50 and a length weight of 3.

The present invention also provides isolated nucleic acid molecules that contain a fragment or portion that hybridizes under highly stringent conditions to a nucleotide sequence comprising a nucleotide sequence selected from the group consisting of SEQ ID NOs: 1, 114-258, or the complement of such a sequence, and also provides isolated nucleic acid molecules that contain a fragment or portion that hybridizes under highly stringent conditions to a nucleotide sequence encoding an amino acid sequence or polymorphic variant thereof. The nucleic acid fragments of the invention are at least about 15, preferably at least about 18, 20, 23 or 25 nucleotides, and can be 30, 40, 50, 100, 200 or more nucleotides in length. Longer fragments, for example, 30 or more nucleotides in length, that encode antigenic polypeptides described herein are particularly useful, such as for the generation of antibodies as described below.

Probes and Primers

In a related aspect, the nucleic acid fragments of the invention are used as probes or primers in assays such as those described herein. “Probes” or “primers” are oligonucleotides that hybridize in a base-specific manner to a complementary strand of nucleic acid molecules. Such probes and primers include polypeptide nucleic acids, as described in Nielsen et al., Science 254:1497-1500 (1991).

A probe or primer comprises a region of nucleotide sequence that hybridizes to at least about 15, for example about 20-25, and in certain embodiments about 40, 50 or 75, consecutive nucleotides of a nucleic acid molecule comprising a contiguous nucleotide sequence selected from the group consisting of SEQ ID NOs: 1, 114-258 or polymorphic variant thereof. In other embodiments, a probe or primer comprises 100 or fewer nucleotides, in certain embodiments from 6 to 50 nucleotides, for example from 12 to 30 nucleotides. In other embodiments, the probe or primer is at least 70% identical to the contiguous nucleotide sequence or to the complement of the contiguous nucleotide sequence, for example at least 80% identical, in certain embodiments at least 90% identical, and in other embodiments at least 95% identical, or even capable of selectively hybridizing to the contiguous nucleotide sequence or to the complement of the contiguous nucleotide sequence. Often, the probe or primer further comprises a label, e.g., radioisotope, fluorescent compound, enzyme, or enzyme co-factor.

The nucleic acid molecules of the invention such as those described above can be identified and isolated using standard molecular biology techniques and the sequence information provided herein. For example, nucleic acid molecules can be amplified and isolated by the polymerase chain reaction using synthetic oligonucleotide primers designed based on one or more of the sequences selected from the group consisting of SEQ ID NOs: 1, 114-258 or the complement of such a sequence, or designed based on nucleotides based on sequences encoding one or more of the amino acid sequences provided herein. See generally PCR Technology: Principles and Applications for DNA Amplification (ed. H. A. Erlich, Freeman Press, NY, N.Y., 1992); PCR Protocols: A Guide to Methods and Applications (Eds. Innis et al., Academic Press, San Diego, Calif., 1990); Mattila et al., Nucl. Acids Res. 19: 4967 (1991); Eckert et al., PCR Methods and Applications 1:17 (1991); PCR (eds. McPherson et al., IRL Press, Oxford); and U.S. Pat. No. 4,683,202. The nucleic acid molecules can be amplified using cDNA, mRNA or genomic DNA as a template, cloned into an appropriate vector and characterized by DNA sequence analysis.

Other suitable amplification methods include the ligase chain reaction (LCR) (see Wu and Wallace, Genomics 4:560 (1989), Landegren et al., Science 241:1077 (1988), transcription amplification (Kwoh et al., Proc. Natl. Acad. Sci. USA 86:1173 (1989)), and self-sustained sequence replication (Guatelli et al., Proc. Nat. Acad. Sci. USA 87:1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The amplified DNA can be labeled, for example, radiolabeled, and used as a probe for screening a cDNA library derived from human cells, mRNA in zap express, ZIPLOX or other suitable vector. Corresponding clones can be isolated, DNA can obtained following in vivo excision, and the cloned insert can be sequenced in either or both orientations by art recognized methods to identify the correct reading frame encoding a polypeptide of the appropriate molecular weight. For example, the direct analysis of the nucleotide sequence of nucleic acid molecules of the present invention can be accomplished using well-known methods that are commercially available. See, for example, Sambrook et al., Molecular Cloning, A Laboratory Manual (2nd Ed., CSHP, New York 1989); Zyskind et al., Recombinant DNA Laboratory Manual, (Acad. Press, 1988)). Additionally, fluorescence methods are also available for analyzing nucleic acids (Chen et al., Genome Res. 9, 492 (1999)) and polypeptides. Using these or similar methods, the polypeptide and the DNA encoding the polypeptide can be isolated, sequenced and further characterized.

Antisense nucleic acid molecules of the invention can be designed using the nucleotide sequences of one or more of SEQ ID NOs: 1, 114-258 and/or the complement of one or more of SEQ ID NOs: 1, 114-258 and/or a portion of one or more of SEQ ID NOs: 1, 114-258 or the complement of one or more of SEQ ID NOs: 1, 114-258 and constructed using chemical synthesis and enzymatic ligation reactions using procedures known in the art. For example, an antisense nucleic acid molecule (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Alternatively, the antisense nucleic acid molecule can be produced biologically using an expression vector into which a nucleic acid molecule has been subcloned in an antisense orientation (i.e., RNA transcribed from the inserted nucleic acid molecule will be of an antisense orientation to a target nucleic acid of interest).

The nucleic acid sequences can also be used to compare with endogenous DNA sequences in patients to identify one or more of the disorders described above, and as probes, such as to hybridize and discover related DNA sequences or to subtract out known sequences from a sample. The nucleic acid sequences can further be used to derive primers for genetic fingerprinting, to raise anti-polypeptide antibodies using DNA immunization techniques, and as an antigen to raise anti-DNA antibodies or elicit immune responses. Portions or fragments of the nucleotide sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Additionally, the nucleotide sequences of the invention can be used to identify and express recombinant polypeptides for analysis, characterization or therapeutic use, or as markers for tissues in which the corresponding polypeptide is expressed, either constitutively, during tissue differentiation, or in diseased states. The nucleic acid sequences can additionally be used as reagents in the screening and/or diagnostic assays described herein, and can also be included as components of kits (e.g., reagent kits) for use in the screening and/or diagnostic assays described herein.

Vectors and Host Cells

Another aspect of the invention pertains to nucleic acid constructs containing a nucleic acid molecule selected from the group consisting of SEQ ID NOs: 1, 114-258 and the complements thereof (or a portion thereof). The constructs comprise a vector (e.g., an expression vector) into which a sequence of the invention has been inserted in a sense or antisense orientation. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Expression vectors are capable of directing the expression of genes to which they are operably linked. In general, expression vectors of utility in recombinant DNA techniques are often in the form of plasmids. However, the invention is intended to include such other forms of expression vectors, such as viral vectors (e.g., replication defective retroviruses, adenoviruses and adeno-associated viruses) that serve equivalent functions.

In certain embodiments, recombinant expression vectors of the invention comprise a nucleic acid molecule of the invention in a form suitable for expression of the nucleic acid molecule in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” or “operatively linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, “Gene Expression Technology”, Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed and the level of expression of polypeptide desired. The expression vectors of the invention can be introduced into host cells to thereby produce polypeptides, including fusion polypeptides, encoded by nucleic acid molecules as described herein.

The recombinant expression vectors of the invention can be designed for expression of a polypeptide of the invention in prokaryotic or eukaryotic cells, e.g., bacterial cells such as E. coli, insect cells (using baculovirus expression vectors), yeast cells or mammalian cells. Suitable host cells are discussed further in Goeddel, supra. Alternatively, the recombinant expression vector can be transcribed and translated in vitro, for example using T7 promoter regulatory sequences and T7 polymerase.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a nucleic acid molecule of the invention can be expressed in bacterial cells (e.g., E. coli), insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing a foreign nucleic acid molecule (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al., (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those that confer resistance to drugs, such as G418, hygromycin and methotrexate. Nucleic acid molecules encoding a selectable marker can be introduced into a host cell on the same vector as the nucleic acid molecule of the invention or can be introduced on a separate vector. Cells stably transfected with the introduced nucleic acid molecule can be identified by drug selection (e.g., cells that have incorporated the selectable marker gene will survive, while the other cells die).

A host cell of the invention, such as a prokaryotic or eukaryotic host cell in culture, can be used to produce (i.e., express) a polypeptide of the invention. Accordingly, the invention further provides methods for producing a polypeptide using the host cells of the invention. In one embodiment, the method comprises culturing the host cell of invention (into which a recombinant expression vector encoding a polypeptide of the invention has been introduced) in a suitable medium such that the polypeptide is produced. In another embodiment, the method further comprises isolating the polypeptide from the medium or the host cell.

The host cells of the invention can also be used to produce nonhuman transgenic animals. For example, in one embodiment, a host cell of the invention is a fertilized oocyte or an embryonic stem cell into which a nucleic acid molecule of the invention has been introduced (e.g., an exogenous KChIP1 gene, or an exogenous nucleic acid encoding a KChIP1 polypeptide). Such host cells can then be used to create non-human transgenic animals in which exogenous nucleotide sequences have been introduced into the genome or homologous recombinant animals in which endogenous nucleotide sequences have been altered. Such animals are useful for studying the function and/or activity of the nucleotide sequence and polypeptide encoded by the sequence and for identifying and/or evaluating modulators of their activity. As used herein, a “transgenic animal” is a non-human animal, preferably a mammal, more preferably a rodent such as a rat or mouse, in which one or more of the cells of the animal include a transgene. Other examples of transgenic animals include non-human primates, sheep, dogs, cows, goats, chickens and amphibians. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo (Cold Spring Harbor Laboratory Press, Cold Spring Harbor, N.Y., 1986). Methods for constructing homologous recombination vectors and homologous recombinant animals are described further in Bradley, Current Opinion in BioTechnology 2:823-829 (1991) and in PCT Publication Nos. WO 90/11354, WO 91/01140, WO 92/0968, and WO 93/04169. Clones of the non-human transgenic animals described herein can also be produced according to the methods described in Wilmut et al., Nature 385:810-813 (1997) and PCT Publication Nos. WO 97/07668 and WO 97/07669.

Polypeptides of the Invention

The present invention also pertains to isolated polypeptides encoded by KChIP1 nucleic acids (“KChIP1 polypeptides,” or “KChIP1 proteins,” such as the protein shown in SEQ ID NO: 2) and fragments and variants thereof, as well as polypeptides encoded by nucleotide sequences described herein (e.g., other splicing variants). The term “polypeptide” refers to a polymer of amino acids, and not to a specific length; thus, peptides, oligopeptides and proteins are included within the definition of a polypeptide. As used herein, a polypeptide is said to be “isolated” or “purified” when it is substantially free of cellular material when it is isolated from recombinant and non-recombinant cells, or free of chemical precursors or other chemicals when it is chemically synthesized. A polypeptide, however, can be joined to another polypeptide with which it is not normally associated in a cell (e.g., in a “fusion protein”) and still be “isolated” or “purified.”

The polypeptides of the invention can be purified to homogeneity. It is understood, however, that preparations in which the polypeptide is not purified to homogeneity are useful. The critical feature is that the preparation allows for the desired function of the polypeptide, even in the presence of considerable amounts of other components. Thus, the invention encompasses various degrees of purity. In one embodiment, the language “substantially free of cellular material” includes preparations of the polypeptide having less than about 30% (by dry weight) other proteins (i.e., contaminating protein), less than about 20% other proteins, less than about 10% other proteins, or less than about 5% other proteins.

When a polypeptide is recombinantly produced, it can also be substantially free of culture medium, i.e., culture medium represents less than about 20%, less than about 10%, or less than about 5% of the volume of the polypeptide preparation. The language “substantially free of chemical precursors or other chemicals” includes preparations of the polypeptide in which it is separated from chemical precursors or other chemicals that are involved in its synthesis. In one embodiment, the language “substantially free of chemical precursors or other chemicals” includes preparations of the polypeptide having less than about 30% (by dry weight) chemical precursors or other chemicals, less than about 20% chemical precursors or other chemicals, less than about 10% chemical precursors or other chemicals, or less than about 5% chemical precursors or other chemicals.

In one embodiment, a polypeptide of the invention comprises an amino acid sequence encoded by a nucleic acid molecule comprising a nucleotide sequence of SEQ ID NO: 1, optionally additionally comprising one or more of SEQ ID NOs: 114-258; or the complement of such a nucleic acid, or portions thereof, or a portion or polymorphic variant thereof. However, the polypeptides of the invention also encompass fragment and sequence variants. Variants include a substantially homologous polypeptide encoded by the same genetic locus in an organism, i.e., an allelic variant, as well as other splicing variants. Variants also encompass polypeptides derived from other genetic loci in an organism, but having substantial homology to a polypeptide encoded by a nucleic acid molecule comprising a nucleotide of SEQ ID NO: 1, optionally additionally one or more of SEQ ID NOs: 114-258; or a complement of such a sequence, or portions thereof or polymorphic variants thereof. Variants also include polypeptides substantially homologous or identical to these polypeptides but derived from another organism, i.e., an ortholog. Variants also include polypeptides that are substantially homologous or identical to these polypeptides that are produced by chemical synthesis. Variants also include polypeptides that are substantially homologous or identical to these polypeptides that are produced by recombinant methods.

As used herein, two polypeptides (or a region of the polypeptides) are substantially homologous or identical when the amino acid sequences are at least about 45-55%, in certain embodiments at least about 70-75%, and in other embodiments at least about 80-85%, and in other embodiments greater than about 90% or more homologous or identical. A substantially homologous amino acid sequence, according to the present invention, will be encoded by a nucleic acid molecule hybridizing to of SEQ ID NO: 1 or any one of 114-258 or portion thereof, under stringent conditions as more particularly described above, or will be encoded by a nucleic acid molecule hybridizing to a nucleic acid sequence encoding SEQ ID NO: 1 or any one of 114-258 or a portion thereof or polymorphic variant thereof, under stringent conditions as more particularly described thereof.

The invention also encompasses polypeptides having a lower degree of identity but having sufficient similarity so as to perform one or more of the same functions performed by a polypeptide encoded by a nucleic acid molecule of the invention.

Similarity is determined by conserved amino acid substitution where a given amino acid in a polypeptide is substituted by another amino acid of like characteristics. Conservative substitutions are likely to be phenotypically silent. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe and Tyr. Guidance concerning which amino acid changes are likely to be phenotypically silent are found in Bowie et al., Science 247:1306-1310 (1990).

A variant polypeptide can differ in amino acid sequence by one or more substitutions, deletions, insertions, inversions, fusions, and truncations or a combination of any of these. Further, variant polypeptides can be fully functional or can lack function in one or more activities. Fully functional variants typically contain only conservative variation or variation in non-critical residues or in non-critical regions. Functional variants can also contain substitution of similar amino acids that result in no change or an insignificant change in function. Alternatively, such substitutions may positively or negatively affect function to some degree. Non-functional variants typically contain one or more non-conservative amino acid substitutions, deletions, insertions, inversions, or truncation or a substitution, insertion, inversion, or deletion in a critical residue or critical region.

Amino acids that are essential for function can be identified by methods known in the art, such as site-directed mutagenesis or alanine-scanning mutagenesis (Cunningham et al., Science 244:1082-1185 (1989)). The latter procedure introduces single alanine mutations at every residue in the molecule. The resulting mutant molecules are then tested for biological activity in vitro, or in vitro proliferative activity. Sites that are critical for polypeptide activity can also be determined by structural analysis such as crystallization, nuclear magnetic resonance or photoaffinity labeling (Smith et al., J. Mol. Biol. 224:899-904 (1992); de Vos et al., Science 255:306-312 (1992)).

The invention also includes polypeptide fragments of the polypeptides of the invention. Fragments can be derived from a polypeptide encoded by a nucleic acid molecule comprising SEQ ID NO: 1 and optionally comprising one or more of SEQ ID NOs: 114-258; or a complement of such a nucleic acid or other variants. However, the invention also encompasses fragments of the variants of the polypeptides described herein. As used herein, a fragment comprises at least 6 contiguous amino acids. Useful fragments include those that retain one or more of the biological activities of the polypeptide as well as fragments that can be used as an immunogen to generate polypeptide-specific antibodies.

Biologically active fragments (peptides which are, for example, 6, 9, 12, 15, 16, 20, 30, 35, 36, 37, 38, 39, 40, 50, 100 or more amino acids in length) can comprise a domain, segment, or motif that has been identified by analysis of the polypeptide sequence using well-known methods, e.g., signal peptides, extracellular domains, one or more transmembrane segments or loops, ligand binding regions, zinc finger domains, DNA binding domains, acylation sites, glycosylation sites, or phosphorylation sites.

Fragments can be discrete (not fused to other amino acids or polypeptides) or can be within a larger polypeptide. Further, several fragments can be comprised within a single larger polypeptide. In one embodiment a fragment designed for expression in a host can have heterologous pre- and pro-polypeptide regions fused to the amino terminus of the polypeptide fragment and an additional region fused to the carboxyl terminus of the fragment.

The invention thus provides chimeric or fusion polypeptides. These comprise a polypeptide of the invention operatively linked to a heterologous protein or polypeptide having an amino acid sequence not substantially homologous to the polypeptide.

“Operatively linked” indicates that the polypeptide and the heterologous protein are fused in-frame. The heterologous protein can be fused to the N-terminus or C-terminus of the polypeptide. In one embodiment the fusion polypeptide does not affect function of the polypeptide per se. For example, the fusion polypeptide can be a GST-fusion polypeptide in which the polypeptide sequences are fused to the C-terminus of the GST sequences. Other types of fusion polypeptides include, but are not limited to, enzymatic fusion polypeptides, for example beta-galactosidase fusions, yeast two-hybrid GAL fusions, poly-His fusions and Ig fusions. Such fusion polypeptides, particularly poly-His fusions, can facilitate the purification of recombinant polypeptide. In certain host cells (e.g., mammalian host cells), expression and/or secretion of a polypeptide can be increased using a heterologous signal sequence. Therefore, in another embodiment, the fusion polypeptide contains a heterologous signal sequence at its N-terminus.

EP-A-O 464 533 discloses fusion proteins comprising various portions of immunoglobulin constant regions. The Fc is useful in therapy and diagnosis and thus results, for example, in improved pharmacokinetic properties (EP-A 0232 262). In drug discovery, for example, human proteins have been fused with Fc portions for the purpose of high-throughput screening assays to identify antagonists. Bennett et al., Journal of Molecular Recognition, 8:52-58 (1995) and Johanson et al., The Journal of Biological Chemistry, 270, 16:9459-9471 (1995). Thus, this invention also encompasses soluble fusion polypeptides containing a polypeptide of the invention and various portions of the constant regions of heavy or light chains of immunoglobulins of various subclasses (IgG, IgM, IgA, IgE).

A chimeric or fusion polypeptide can be produced by standard recombinant DNA techniques. For example, DNA fragments coding for the different polypeptide sequences are ligated together in-frame in accordance with conventional techniques. In another embodiment, the fusion gene can be synthesized by conventional techniques including automated DNA synthesizers. Alternatively, PCR amplification of nucleic acid fragments can be carried out using anchor primers which give rise to complementary overhangs between two consecutive nucleic acid fragments which can subsequently be annealed and re-amplified to generate a chimeric nucleic acid sequence (see Ausubel et al., Current Protocols in Molecular Biology, 1992).

Moreover, many expression vectors are commercially available that already encode a fusion moiety (e.g., a GST protein). A nucleic acid molecule encoding a polypeptide of the invention can be cloned into such an expression vector such that the fusion moiety is linked in-frame to the polypeptide.

The isolated polypeptide can be purified from cells that naturally express it, can be purified from cells that have been altered to express it (recombinant), or synthesized using known protein synthesis methods. In one embodiment, the polypeptide is produced by recombinant DNA techniques. For example, a nucleic acid molecule encoding the polypeptide is cloned into an expression vector, the expression vector introduced into a host cell and the polypeptide expressed in the host cell. The polypeptide can then be isolated from the cells by an appropriate purification scheme using standard protein purification techniques.

The polypeptides of the present invention can be used to raise antibodies or to elicit an immune response. The polypeptides can also be used as a reagent, e.g., a labeled reagent, in assays to quantitatively determine levels of the polypeptide or a molecule to which it binds (e.g., a ligand) in biological fluids. The polypeptides can also be used as markers for cells or tissues in which the corresponding polypeptide is preferentially expressed, either constitutively, during tissue differentiation, or in a diseased state. The polypeptides can be used to isolate a corresponding binding agent, e.g., ligand or receptor, such as, for example, in an interaction trap assay, and to screen for peptide or small molecule antagonists or agonists of the binding interaction.

Antibodies of the Invention

Polyclonal antibodies and/or monoclonal antibodies that specifically bind one form of the gene product but not to the other form of the gene product are also provided. Antibodies are also provided which bind a portion of either the variant or the reference gene product that contains the polymorphic site or sites. The term “antibody” as used herein refers to immunoglobulin molecules and immunologically active portions of immunoglobulin molecules, i.e., molecules that contain an antigen binding site that specifically bind an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or a fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein, Nature 256:495-497 (1975), the human B cell hybridoma technique (Kozbor et al., Immunol. Today 4: 72 (1983)), the EBV-hybridoma technique (Cole et al, Monoclonal Antibodies and Cancer Therapy, Alan R. Liss, 1985, Inc., pp. 77-96) or trioma techniques. The technology for producing hybridomas is well known (see generally Current Protocols in Immunology (1994) Coligan et al., (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al., Nature 266:55052 (1977); R. H. Kenneth, in Monoclonal Antibodies: A New Dimension In Biological Analyses, Plenum Publishing Corp., New York, N.Y. (1980); and Lerner, Yale J. Biol. Med. 54:387-402 (1981)). Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog NO: 27-9400-01; and the Stratagene SurfZAP™ Phage Display Kit, Catalog NO: 240612). Additionally, examples of methods and reagents particularly amenable for use in generating and screening antibody display library can be found in, for example, U.S. Pat. No. 5,223,409; PCT Publication NO: WO 92/18619; PCT Publication NO: WO 91/17271; PCT Publication NO: WO 92/20791; PCT Publication NO: WO 92/15679; PCT Publication NO: WO 93/01288; PCT Publication NO: WO 92/01047; PCT Publication NO: WO 92/09690; PCT Publication NO: WO 90/02809; Fuchs et al., Bio/Technology 9: 1370-1372 (1991); Hay et al., Hum. Antibod. Hybridomas 3:81-85 (1992); Huse et al., Science 246: 1275-1281 (1989); and Griffiths et al., EMBO J. 12:725-734 (1993).

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide of the invention by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue as part of a clinical testing procedure, e.g., to, for example, determine the efficacy of a given treatment regimen. The antibody can be coupled to a detectable substance to facilitate its detection. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Diagnostic Assays

The nucleic acids, probes, primers, polypeptides and antibodies described herein can be used in methods of diagnosis of Type II diabetes; of a susceptibility to Type II diabetes; or of a condition associated with a KChIP1 gene, as well as in kits (e.g., useful for diagnosis of Type II diabetes; a susceptibility to Type II diabetes; or a condition associated with a KChIP1 gene). In one embodiment, the kit comprises primers which contain one or more of the SNP's identified in Table 10.

In one embodiment of the invention, diagnosis of a disease or condition associated with a KChIP1 gene (e.g., diagnosis of Type II diabetes, or of a susceptibility to Type II diabetes) is made by detecting a polymorphism in a KChIP1 nucleic acid as described herein. The polymorphism can be a change in a KChIP1 nucleic acid, such as the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of the gene; duplication of all or a part of the gene; transposition of all or a part of the gene; or rearrangement of all or a part of the gene. More than one such change may be present in a single gene. Such sequence changes cause a difference in the polypeptide encoded by a KChIP1 nucleic acid. For example, if the difference is a frame shift change, the frame shift can result in a change in the encoded amino acids, and/or can result in the generation of a premature stop codon, causing generation of a truncated polypeptide. Alternatively, a polymorphism associated with a disease or condition or a susceptibility to a disease or condition associated with a KChIP1 nucleic acid can be a synonymous alteration in one or more nucleotides (i.e., an alteration that does not result in a change in the polypeptide encoded by a KChIP1 nucleic acid). Such a polymorphism may alter splicing sites, affect the stability or transport of mRNA, or otherwise affect the transcription or translation of the gene. A KChIP1 nucleic acid that has any of the changes or alterations described above is referred to herein as an “altered nucleic acid.”

In a first method of diagnosing Type II diabetes or a susceptibility to Type II diabetes, or another disease or condition associated with a KChIP1 gene, hybridization methods, such as Southern analysis, Northern analysis, or in situ hybridizations, can be used (see Current Protocols in Molecular Biology, Ausubel, F. et al., eds, John Wiley & Sons, including all supplements through 1999). For example, a biological sample (a “test sample”) from a test subject (the “test individual”) of genomic DNA, RNA, or cDNA, is obtained from an individual, such as an individual suspected of having, being susceptible to or predisposed for, or carrying a defect for, the disease or condition, or the susceptibility to the disease or condition, associated with a KChIP1 gene (e.g., Type II diabetes). The individual can be an adult, child, or fetus. The test sample can be from any source which contains genomic DNA, such as a blood sample, sample of amniotic fluid, sample of cerebrospinal fluid, or tissue sample from skin, muscle, buccal or conjunctival mucosa, placenta, gastrointestinal tract or other organs. A test sample of DNA from fetal cells or tissue can be obtained by appropriate methods, such as by amniocentesis or chorionic villus sampling. The DNA, RNA, or cDNA sample is then examined to determine whether a polymorphism in a KChIP1 nucleic acid is present, and/or to determine which splicing variant(s) encoded by the KChIP1 is present. The presence of the polymorphism or splicing variant(s) can be indicated by hybridization of the gene in the genomic DNA, RNA, or cDNA to a nucleic acid probe. A “nucleic acid probe”, as used herein, can be a DNA probe or an RNA probe; the nucleic acid probe can contain, for example, at least one polymorphism in a KChIP1 nucleic acid (e.g., as set forth in Table 10) and/or contain a nucleic acid encoding a particular splicing variant of a KChIP1 nucleic acid. The probe can be any of the nucleic acid molecules described above (e.g., the gene or nucleic acid, a fragment, a vector comprising the gene or nucleic acid, a probe or primer, etc.).

To diagnose Type II diabetes, or a susceptibility to Type II diabetes, or another condition associated with a KChIP1 gene, a hybridization sample is formed by contacting the test sample containing a KChIP1 nucleic acid with at least one nucleic acid probe. A preferred probe for detecting mRNA or genomic DNA is a labeled nucleic acid probe capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate mRNA or genomic DNA. For example, the nucleic acid probe can be all or a portion of one of SEQ ID NOs: 114-258 or the complement thereof, or a portion thereof. Other suitable probes for use in the diagnostic assays of the invention are described above (see e.g., probes and primers discussed under the heading, “Nucleic Acids of the Invention”).

The hybridization sample is maintained under conditions that are sufficient to allow specific hybridization of the nucleic acid probe to a KChIP1 nucleic acid. “Specific hybridization”, as used herein, indicates exact hybridization (e.g., with no mismatches). Specific hybridization can be performed under high stringency conditions or moderate stringency conditions, for example, as described above. In a particularly preferred embodiment, the hybridization conditions for specific hybridization are high stringency.

Specific hybridization, if present, is then detected using standard methods. If specific hybridization occurs between the nucleic acid probe and KChIP1 nucleic acid in the test sample, then the KChIP1 has the polymorphism, or is the splicing variant, that is present in the nucleic acid probe. More than one nucleic acid probe can also be used concurrently in this method. Specific hybridization of any one of the nucleic acid probes is indicative of a polymorphism in the KChIP1 nucleic acid, or of the presence of a particular splicing variant encoding the KChIP1 nucleic acid and is therefore diagnostic for a susceptibility to a disease or condition associated with a KChIP1 nucleic acid (e.g., Type II diabetes).

In Northern analysis (see Current Protocols in Molecular Biology, Ausubel, F. et al, eds., John Wiley & Sons, supra) the hybridization methods described above are used to identify the presence of a polymorphism or a particular splicing variant, associated with a susceptibility to a disease or condition associated with a KChIP1 gene (e.g., Type II diabetes). For Northern analysis, a test sample of RNA is obtained from the individual by appropriate means. Specific hybridization of a nucleic acid probe, as described above, to RNA from the individual is indicative of a polymorphism in a KChIP1 nucleic acid, or of the presence of a particular splicing variant encoded by a KChIP1 nucleic acid and is therefore diagnostic for Type II diabetes or a susceptibility to Type II diabetes or a condition associated with a KChIP1 nucleic acid (e.g., Type II diabetes).

For representative examples of use of nucleic acid probes, see, for example, U.S. Pat. Nos. 5,288,611 and 4,851,330.

Alternatively, a peptide nucleic acid (PNA) probe can be used instead of a nucleic acid probe in the hybridization methods described above. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P. E. et al., Bioconjugate Chemistry 5, American Chemical Society, p. 1 (1994). The PNA probe can be designed to specifically hybridize to a gene having a polymorphism associated with a susceptibility to a disease or condition associated with a KChIP1 nucleic acid (e.g., Type II diabetes). Hybridization of the PNA probe to a KChIP1 gene is diagnostic for Type II diabetes or a susceptibility to Type II diabetes or a condition associated with a KChIP1 nucleic acid.

In another method of the invention, alteration analysis by restriction digestion can be used to detect an altered gene, or genes containing a polymorphism(s), if the alteration (mutation) or polymorphism in the gene results in the creation or elimination of a restriction site. A test sample containing genomic DNA is obtained from the individual. Polymerase chain reaction (PCR) can be used to amplify a KChIP1 nucleic acid (and, if necessary, the flanking sequences) in the test sample of genomic DNA from the test individual. RFLP analysis is conducted as described (see Current Protocols in Molecular Biology, supra). The digestion pattern of the relevant DNA fragment indicates the presence or absence of the alteration or polymorphism in the KChIP1 nucleic acid, and therefore indicates the presence or absence of Type II diabetes or the susceptibility to a disease or condition associated with a KChIP1 nucleic acid.

Sequence analysis can also be used to detect specific polymorphisms in a KChIP1 nucleic acid. A test sample of DNA or RNA is obtained from the test individual. PCR or other appropriate methods can be used to amplify the gene or nucleic acid, and/or its flanking sequences, if desired. The sequence of a KChIP1 nucleic acid, or a fragment of the nucleic acid, or cDNA, or fragment of the cDNA, or mRNA, or fragment of the mRNA, is determined, using standard methods. The sequence of the nucleic acid, nucleic acid fragment, cDNA, cDNA fragment, mRNA, or mRNA fragment is compared with the known nucleic acid sequence of the gene or cDNA (e.g., one or more of SEQ ID NOs: 114-258 or a complement thereof) or mRNA, as appropriate. The presence of a polymorphism in the KChIP1 indicates that the individual has Type II diabetes or a susceptibility to Type II diabetes.

Allele-specific oligonucleotides can also be used to detect the presence of a polymorphism in a KChIP1 nucleic acid, through the use of dot-blot hybridization of amplified oligonucleotides with allele-specific oligonucleotide (ASO) probes (see, for example, Saiki, R. et al., Nature 324:163-166 (1986)). An “allele-specific oligonucleotide” (also referred to herein as an “allele-specific oligonucleotide probe”) is an oligonucleotide of approximately 10-50 base pairs, preferably approximately 15-30 base pairs, that specifically hybridizes to a KChIP1 nucleic acid, and that contains a polymorphism associated with a susceptibility to a disease or condition associated with a KChIP1 nucleic acid. An allele-specific oligonucleotide probe that is specific for particular polymorphisms in a KChIP1 nucleic acid can be prepared, using standard methods (see Current Protocols in Molecular Biology, supra). To identify polymorphisms in the gene that are associated with a disease or condition associated with a KChIP1 nucleic acid or a susceptibility to a disease or condition associated with a KChIP1 nucleic acid a test sample of DNA is obtained from the individual. PCR can be used to amplify all or a fragment of a KChIP1 nucleic acid and its flanking sequences. The DNA containing the amplified KChIP1 nucleic acid (or fragment of the gene or nucleic acid) is dot-blotted, using standard methods (see Current Protocols in Molecular Biology, supra), and the blot is contacted with the oligonucleotide probe. The presence of specific hybridization of the probe to the amplified KChIP1 nucleic acid is then detected. Hybridization of an allele-specific oligonucleotide probe to DNA from the individual is indicative of a polymorphism in the KChIP1 nucleic acid, and is therefore indicative of a disease or condition associated with a KChIP1 nucleic acid or susceptibility to a disease or condition associated with a KChIP1 nucleic acid (e.g., Type II diabetes).

The invention further provides allele-specific oligonucleotides that hybridize to the reference or variant allele of a gene or nucleic acid comprising a single nucleotide polymorphism or to the complement thereof. These oligonucleotides can be probes or primers.

An allele-specific primer hybridizes to a site on target DNA overlapping a polymorphism and only primes amplification of an allelic form to which the primer exhibits perfect complementarity. See Gibbs, Nucleic Acid Res. 17, 2427-2448 (1989). This primer is used in conjunction with a second primer, which hybridizes at a distal site. Amplification proceeds from the two primers, resulting in a detectable product, which indicates the particular allelic form is present. A control is usually performed with a second pair of primers, one of which shows a single base mismatch at the polymorphic site and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing to elongation from the primer (see, e.g., WO 93/22456).

With the addition of such analogs as locked nucleic acids (LNAs), the size of primers and probes can be reduced to as few as 8 bases. LNAs are a novel class of bicyclic DNA analogs in which the 2′ and 4′ positions in the furanose ring are joined via an O-methylene (oxy-LNA), S-methylene (thio-LNA), or amino methylene (amino-LNA) moiety. Common to all of these LNA variants is an affinity toward complementary nucleic acids, which is by far the highest reported for a DNA analog. For example, particular all oxy-LNA nonamers have been shown to have melting temperatures of 64° C. and 74° C. when in complex with complementary DNA or RNA, respectively, as oposed to 28° C. for both DNA and RNA for the corresponding DNA nonamer. Substantial increases in Tₘ are also obtained when LNA monomers are used in combination with standard DNA or RNA monomers. For primers and probes, depending on where the LNA monomers are included (e.g., the 3′ end, the 5′end, or in the middle), the Tₘ could be increased considerably.

In another embodiment, arrays of oligonucleotide probes that are complementary to target nucleic acid sequence segments from an individual, can be used to identify polymorphisms in a KChIP1 nucleic acid. For example, in one embodiment, an oligonucleotide array can be used. Oligonucleotide arrays typically comprise a plurality of different oligonucleotide probes that are coupled to a surface of a substrate in different known locations. These oligonucleotide arrays, also described as “Genechips™,” have been generally described in the art, for example, U.S. Pat. No. 5,143,854 and PCT patent publication Nos. WO 90/15070 and 92/10092. These arrays can generally be produced using mechanical synthesis methods or light directed synthesis methods that incorporate a combination of photolithographic methods and solid phase oligonucleotide synthesis methods. See Fodor et al., Science 251:767-777 (1991), Pirrung et al., U.S. Pat. No. 5,143,854 (see also PCT Application NO: WO 90/15070) and Fodor et al., PCT Publication NO: WO 92/10092 and U.S. Pat. No. 5,424,186, the entire teachings of each of which are incorporated by reference herein. Techniques for the synthesis of these arrays using mechanical synthesis methods are described in, e.g., U.S. Pat. No. 5,384,261; the entire teachings of which are incorporated by reference herein. In another example, linear arrays can be utilized.

Once an oligonucleotide array is prepared, a nucleic acid of interest is hybridized with the array and scanned for polymorphisms. Hybridization and scanning are generally carried out by methods described herein and also in, e.g., published PCT Application Nos. WO 92/10092 and WO 95/11995, and U.S. Pat. No. 5,424,186, the entire teachings of which are incorporated by reference herein. In brief, a target nucleic acid sequence that includes one or more previously identified polymorphic markers is amplified by well-known amplification techniques, e.g., PCR. Typically, this involves the use of primer sequences that are complementary to the two strands of the target sequence both upstream and downstream from the polymorphism. Asymmetric PCR techniques may also be used. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

Although primarily described in terms of a single detection block, e.g., for detection of a single polymorphism, arrays can include multiple detection blocks, and thus be capable of analyzing multiple, specific polymorphisms. In alternative arrangements, it will generally be understood that detection blocks may be grouped within a single array or in multiple, separate arrays so that varying, optimal conditions may be used during the hybridization of the target to the array. For example, it may often be desirable to provide for the detection of those polymorphisms that fall within G-C rich stretches of a genomic sequence, separately from those falling in A-T rich segments. This allows for the separate optimization of hybridization conditions for each situation.

Additional uses of oligonucleotide arrays for polymorphism detection can be found, for example, in U.S. Pat. Nos. 5,858,659 and 5,837,832, the entire teachings of which are incorporated by reference herein. Other methods of nucleic acid analysis can be used to detect polymorphisms in a Type II diabetes gene or variants encoding by a Type II diabetes gene. Representative methods include direct manual sequencing (Church and Gilbert, Proc. Natl. Acad. Sci. USA 81:1991-1995 (1988); Sanger, F. et al., Proc. Natl. Acad. Sci. USA 74:5463-5467 (1977); Beavis et al., U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V. C. et al., Proc. Natl. Acad. Sci. USA 86:232-236 (1989)), mobility shift analysis (Orita, M. et al., Proc. Natl. Acad. Sci. USA 86:2766-2770 (1989)), restriction enzyme analysis (Flavell et al., Cell 15:25 (1978); Geever, et al., Proc. Natl. Acad. Sci. USA 78:5081 (1981)); heteroduplex analysis; chemical mismatch cleavage (CMC) (Cotton et al., Proc. Natl. Acad. Sci. USA 85:4397-4401 (1985)); RNase protection assays (Myers, R. M. et al., Science 230:1242 (1985)); use of polypeptides which recognize nucleotide mismatches, such as E. coli mutS protein; allele-specific PCR, for example.

In one embodiment of the invention, diagnosis of a disease or condition associated with a KChIP1 nucleic acid (e.g., Type II diabetes) or a susceptibility to a disease or condition associated with a KChIP1 nucleic acid (e.g., Type II diabetes) can also be made by expression analysis by quantitative PCR (kinetic thermal cycling). This technique, utilizing TaqMan®, can be used to allow the identification of polymorphisms and whether a patient is homozygous or heterozygous. The technique can assess the presence of an alteration in the expression or composition of the polypeptide encoded by a KChIP1 nucleic acid or splicing variants encoded by a KChIP1 nucleic acid. Further, the expression of the variants can be quantified as physically or functionally different.

In another embodiment of the invention, diagnosis of Type II diabetes or a susceptibility to Type II diabetes 9 or a condition associated with a KChIP1 gene) can be made by examining expression and/or composition of a KChIP1 polypeptide, by a variety of methods, including enzyme linked immunosorbent assays (ELISAs), Western blots, immunoprecipitations and immunofluorescence. A test sample from an individual is assessed for the presence of an alteration in the expression and/or an alteration in composition of the polypeptide encoded by a KChIP1 nucleic acid, or for the presence of a particular variant encoded by a KChIP1 nucleic acid. An alteration in expression of a polypeptide encoded by a KChIP1 nucleic acid can be, for example, an alteration in the quantitative polypeptide expression (i.e., the amount of polypeptide produced); an alteration in the composition of a polypeptide encoded by a KChIP1 nucleic acid is an alteration in the qualitative polypeptide expression (e.g., expression of an altered KChIP1 polypeptide or of a different splicing variant). In a preferred embodiment, diagnosis of the disease or condition associated with KChIP1 nucleic acid or a susceptibility to a disease or condition associated with a KChIP1 nucleic acid is made by detecting a particular splicing variant encoded by that KChIP1 nucleic acid, or a particular pattern of splicing variants.

Both such alterations (quantitative and qualitative) can also be present. The term “alteration” in the polypeptide expression or composition, as used herein, refers to an alteration in expression or composition in a test sample, as compared with the expression or composition of polypeptide by a KChIP1 nucleic acid in a control sample. A control sample is a sample that corresponds to the test sample (e.g., is from the same type of cells), and is from an individual who is not affected by a susceptibility to a disease or condition associated with a KChIP1 nucleic acid. An alteration in the expression or composition of the polypeptide in the test sample, as compared with the control sample, is indicative of a susceptibility to a disease or condition associated with a KChIP1 nucleic acid. Similarly, the presence of one or more different splicing variants in the test sample, or the presence of significantly different amounts of different splicing variants in the test sample, as compared with the control sample, is indicative of a disease or condition associated with a KChIP1 nucleic acid or a susceptibility to a disease or condition associated with a KChIP1 nucleic acid. Various means of examining expression or composition of the polypeptide encoded by a KChIP1 nucleic acid can be used, including: spectroscopy, colorimetry, lectrophoresis, isoelectric focusing, and immunoassays (e.g., David et al., U.S. Pat. No. 4,376,110) such as immunoblotting (see also Current Protocols in Molecular Biology, particularly Chapter 10). For example, in one embodiment, an antibody capable of binding to the polypeptide (e.g., as described above), preferably an antibody with a detectable label, can be used. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Western blotting analysis, using an antibody as described above that specifically binds to a polypeptide encoded by an altered KChIP1 nucleic acid (e.g., a KChIP1 nucleic acid having one or more alterations as shown in Table 10), or an antibody that specifically binds to a polypeptide encoded by a non-altered nucleic acid, or an antibody that specifically binds to a particular splicing variant encoded by a nucleic acid, can be used to identify the presence in a test sample of a particular splicing variant or of a polypeptide encoded by a polymorphic or altered KChIP1 nucleic acid, or the absence in a test sample of a particular splicing variant or of a polypeptide encoded by a non-polymorphic or non-altered nucleic acid. The presence of a polypeptide encoded by a polymorphic or altered nucleic acid, or the absence of a polypeptide encoded by a non-polymorphic or non-altered nucleic acid, is diagnostic for a disease or condition associated with a KChIP1 nucleic acid or a susceptibility to a disease or condition associated with a KChIP1 nucleic acid (e.g., Type II diabetes), as is the presence (or absence) of particular splicing variants encoded by the KChIP1 nucleic acid.

In one embodiment of this method, the level or amount of polypeptide encoded by a KChIP1 nucleic acid in a test sample is compared with the level or amount of the polypeptide encoded by the KChIP1 in a control sample. A level or amount of the polypeptide in the test sample that is higher or lower than the level or amount of the polypeptide in the control sample, such that the difference is statistically significant, is indicative of an alteration in the expression of the polypeptide encoded by the KChIP1 nucleic acid, and is diagnostic for a disease or condition associated with a KChIP1 nucleic acid or a susceptibility to a disease or condition associated with that KChIP1 nucleic acid (e.g., Type II diabetes). Alternatively, the composition of the polypeptide encoded by a KChIP1 nucleic acid in a test sample is compared with the composition of the polypeptide encoded by the KChIP1 nucleic acid in a control sample (e.g., the presence of different splicing variants). A difference in the composition of the polypeptide in the test sample, as compared with the composition of the polypeptide in the control sample, is diagnostic for a disease or condition associated with a KChIP1 nucleic acid or a susceptibility to a disease or condition associated with that KChIP1 nucleic acid (e.g., Type II diabetes). In another embodiment, both the level or amount and the composition of the polypeptide can be assessed in the test sample and in the control sample. A difference in the amount or level of the polypeptide in the test sample, compared to the control sample; a difference in composition in the test sample, compared to the control sample; or both a difference in the amount or level, and a difference in the composition, is indicative of a disease or condition associated with a KChIP1 nucleic acid or a susceptibility to a disease or condition associated with that KChIP1 nucleic acid.

The invention further pertains to a method for the diagnosis or identification of a susceptibility to Type II diabetes in an individual, by identifying an at-risk haplotype (e.g., a haplotype comprising a KChIP1 nucleic acid). The KChIP1-associated haplotypes, e.g., those described in Table 2, Table 4, Table 5, Table 14, Table 16, Table 17 and Table 18, describe a set of genetic markers (“alleles”). In a certain embodiment, the haplotype can comprise one or more alleles, two or more alleles, three or more alleles, four or more alleles, or five or more alleles. The genetic markers are particular “alleles” at “polymorphic sites” associated with KChIP1. A nucleotide position at which more than one sequence is possible in a population (either a natural population or a synthetic population, e.g., a library of synthetic molecules), is referred to herein as a “polymorphic site”. Where a polymorphic site is a single nucleotide in length, the site is referred to as a single nucleotide polymorphism (“SNP”). For example, if at a particular chromosomal location, one member of a population has an adenine and another member of the population has a thymine at the same position, then this position is a polymorphic site, and, more specifically, the polymorphic site is a SNP. Polymorphic sites can allow for differences in sequences based on substitutions, insertions or deletions. Each version of the sequence with respect to the polymorphic site is referred to herein as an “allele” of the polymorphic site. Thus, in the previous example, the SNP allows for both an adenine allele and a thymine allele.

Typically, a reference sequence is referred to for a particular sequence. Alleles that differ from the reference are referred to as “variant” alleles. For example, the reference KChIP1 sequence is described herein by SEQ ID NO: 1. The term, “variant KChIP1”, as used herein, refers to a sequence that differs from SEQ ID NO: 1, but is otherwise substantially similar. The genetic markers that make up the haplotypes described herein are KChIP1 variants. The variants of KChIP1 that are used to determine the haplotypes disclosed herein of the present invention are associated with Type II diabetes or a susceptibility to Type II diabetes.

Additional variants can include changes that affect a polypeptide, e.g., the KChIP1 polypeptide. These sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence, as described in detail above. Such sequence changes alter the polypeptide encoded by a KChIP1 nucleic acid. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism associated with Type II diabetes or a susceptibility to Type II diabetes can be a synonymous change in one or more nucleotides (i.e., a change that does not result in a change in the amino acid sequence). Such a polymorphism can, for example, alter splice sites, affect the stability or transport of mRNA, or otherwise affect the transcription or translation of the polypeptide. The polypeptide encoded by the reference nucleotide sequence is the “reference” polypeptide with a particular reference amino acid sequence, and polypeptides encoded by variant alleles are referred to as “variant” polypeptides with variant amino acid sequences.

Haplotypes are a combination of genetic markers, e.g., particular alleles at polymorphic sites. The haplotypes described herein, e.g., having markers such as those shown in Table 10, Table 11, Table 12, Table 13, Table 14, Table 16, Table 17 and Table 18, are found more frequently in individuals with Type II diabetes than in individuals without Type II diabetes. Therefore, these haplotypes have predictive value for detecting Type II diabetes or a susceptibility to Type II diabetes in an individual. The haplotypes described herein are a combination of various genetic markers, e.g., SNPs and microsatellites. Therefore, detecting haplotypes can be accomplished by methods known in the art for detecting sequences at polymorphic sites, such as the methods described above.

Haplotype Screening

In the methods for the diagnosis and identification of susceptibility to Type II diabetes or Type II diabetes in an individual, an at-risk haplotype is identified. In one embodiment, the at-risk haplotype is one which confers a significant risk of Type II diabetes. In one embodiment, significance associated with a haplotype is measured by an odds ratio. In a further embodiment, the significance is measured by a percentage. In one embodiment, a significant risk is measured as an odds ratio of at least about 1.2, including by not limited to: 1.2, 1.3, 1.4, 1.5, 1.6, 1.7, 1.8, and 1.9. In a further embodiment, an odds ratio of at least 1.2 is significant. In a further embodiment, an odds ratio of at least about 1.5 is significant. In a further embodiment, a significant increase in risk is at least about 1.7 is significant. In a further embodiment, a significant increase in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase in risk is at least about 50%. It is understood however, that identifying whether a risk is medically significant may also depend on a variety of factors, including the specific disease, the haplotype, and often, environmental factors.

The invention also pertains to methods of diagnosing Type II diabetes or a susceptibility to Type II diabetes in an individual, comprising screening for an at-risk haplotype in, or comprising portions of, the KChIP1 gene, where the haplotype is more frequently present in an individual susceptible to Type II diabetes (affected), compared to the frequency of its presence in a healthy individual (control), and wherein the presence of the haplotype is indicative of Type II diabetes or susceptibility to Type II diabetes. Standard techniques for genotyping for the presence of SNPs and/or microsatellite markers can be used, such as fluorescent based techniques (Chen, et al., Genome Res. 9, 492 (1999)), PCR, LCR, Nested PCR and other techniques for nucleic acid amplification. In a preferred embodiment, the method comprises assessing in an individual the presence or frequency of SNPs and/or microsatellites in, comprising portions of, the KChIP1 gene, wherein an excess or higher frequency of the SNPs and/or microsatellites compared to a healthy control individual is indicative that the individual has Type II diabetes or is susceptible to Type II diabetes. See, for example, Tables 6, 7, 9, 11, 13 and 14 (below) for SNPs and markers that can form haplotypes that can be used as screening tools. These markers and SNPs can be used to design diagnostic tests for determining Type II diabetes or a susceptibility to Type II diabetes. For example, an at-risk haplotype can include microsatellite markers and/or SNPs such as those set forth in Table 10, Table 11, Table 12 Table 13, Table 14, Table 16, Table 17, Table 18 Tables 20-24 and Table 26. The presence of the haplotype is diagnostic of Type II diabetes or of a susceptibility to Type II diabetes. Haplotype analysis involves defining a candidate susceptibility locus using LOD scores. The defined regions are then ultra-fine mapped with microsatellite markers with an average spacing between markers of less than 100 kb. All usable microsatellite markers that found in public databases and mapped within that region can be used. In addition, microsatellite markers identified within the deCODE genetics sequence assembly of the human genome can be used.

The frequencies of haplotypes in the patient and the control groups using an expectation-maximization algorithm can be estimated (Dempster A. et al., 1977. J. R. Stat. Soc. B, 39:1-389). An implementation of this algorithm that can handle missing genotypes and uncertainty with the phase can be used. Under the null hypothesis, the patients and the controls are assumed to have identical frequencies. Using a likelihood approach, an alternative hypothesis where a candidate at-risk-haplotype, which can include the markers described herein, is allowed to have a higher frequency in patients than controls, while the ratios of the frequencies of other haplotypes are assumed to be the same in both groups is tested. Likelihoods are maximized separately under both hypotheses and a corresponding 1-df likelihood ratio statistics is used to evaluate the statistic significance.

To look for at-risk-haplotypes in the 1-lod drop, for example, association of all possible combinations of genotyped markers is studied, provided those markers span a practical region. The combined patient and control groups can be randomly divided into two sets, equal in size to the original group of patients and controls. The haplotype analysis is then repeated and the most significant p-value registered is determined. This randomization scheme can be repeated, for example, over 100 times to construct an empirical distribution of p-values.

The at-risk haplotypes identified in Table 2 (haplotypes identified as A1, A2, A3, A4, A5, A6, B1, B2, B3, B4 and B5), Table 4 (haplotypes identified as D1 and D2), Table 5 (haplotypes identified as D2, D3, D4, D5 and D6) Table 14 (haplotypes identified as Hap S7 and S7′ (formerly Hap E and Hap E′), Table 16 (identified as F1, F2, G1 and G2), Table 17, Table 18 and Tables 20-24 are associated with Type II diabetes or a susceptibility to Type II diabetes. In certain embodiments, a haplotype associated with Type II diabetes or a susceptibility to Type II diabetes comprises markers DG5S879, DG5S881, D5S2075, DG5S883 and DG5S38 at the 5q35 locus; or DG5S1058 and DG5S37 at the 5q35 locus; or DG5S1058, DG5S37 and DG5S101 at the 5q35 locus; or DG5S881, DG5S1058, D5S2075, DG5S883 and DG5S38 at the 5q35 locus; or DG5S879, DG5S1058 and DG5S37; or DG5S881, D5S2075, DG5S883 and DG5S38 at the 5q35 locus; DG5S953, DG5S955, DG5S13 and DG5S959 at the 5q35 locus; or DG5S888 and DG5S953 at the 5q35 locus; or DG5S953, DG5S955 and DG5S124 at the 5q35 locus; or DG5S888, DG5S44 and DG5S953 at the 5q35 locus; or DG5S953, DG5S955, DG5S13, DG5S123, and DG5S959 at the 5q35 locus. The presence of the haplotype is diagnostic of Type II diabetes or of a susceptibility to Type II diabetes. Also described herein is a haplotype associated with Type II diabetes or a susceptibility to Type II diabetes comprising markers DG5S13, KCP_{—}1152, and D5S625 at the 5q35 locus; the presence of the haplotype is diagnostic of Type II diabetes or of a susceptibility to Type II diabetes. In one particular embodiment, the presence of the −4, 1, 0 haplotype at DG5S13, KCP_{—}1152, and D5S625 is diagnostic of Type II diabetes or of a susceptibility to Type II diabetes. In another embodiment, a haplotype associated with Type II diabetes or a susceptibility to Type II diabetes in an individual, comprises markers DG5S124, KCP_{—}1152, KCP_{—}2649, KPC_{—}4976 and KPC-16152 at the 5q35 locus. In one particular embodiment, the presence of the 0, 1, 1, 3 and 0 haplotype at DG5S124, KCP_{—}1152, KCP_{—}2649, KPC_{—}4976 and KPC-16152 is diagnostic of Type II diabetes or of a susceptibility to Type II diabetes. In another embodiment, a haplotype associated with Type II diabetes or a susceptibility to Type II diabetes in an individual, comprises markers KCP_{—}173982, KCP_{—}15400, and KCP_{—}18069. In one particular embodiment, the presence of the 0, 1, 1 haplotype at KCP_{—}173982, KCP_{—}15400, and KCP_{—}18069 is diagnostic of Type II diabetes or of a susceptibility to Type II diabetes.

In additional embodiments, a haplotype associated with Type II diabetes or a susceptibility to Type II diabetes comprises markers DG5S124, KCP_{—}1152, KCP_{—}2649, KCP_{—}4976, and KCP_{—}16152 at the 5q35 locus, as well as one of the following 3 markers: KCP_{—}197678, KCP_{—}197775, and KCP_{—}202795 at the 5q35 locus; the presence of the haplotype is diagnostic of Type II diabetes or of a susceptibility to Type II diabetes. In particular embodiments, the presence of the 0, 3, 1, 1, 3, 0 haplotype at DG5S124, KCP_{—}197678, KCP_{—}1152, KCP_{—}2649, KCP_{—}4976, and KCP_{—}16152; the presence of the 0, 3, 1, 1, 3, 0 haplotype at DG5S124, KCP_{—}197775, KCP_{—}1152, KCP_{—}2649, KCP_{—}4976, and KCP_{—}16152; or the presence of the 0, 1, 1, 1, 3, 0 haplotype at DG5S 124, KCP_{—}202795, KCP_{—}1152, KCP_{—}2649, KCP_{—}4976, and KCP_{—}16152; is diagnostic of Type II diabetes or of a susceptibility to Type II diabetes.

In additional embodiments, a haplotype associated with Type II diabetes or a susceptibility to Type II diabetes comprises markers rs1032856, KCP_RS888934, KCP_{—}93545, KCP_{—}102882, 169234, KCP_{—}186048 and KCP_{—}16152, as well as markers rs1032856, KCP_RS888934, KCP_{—}93545, KCP_{—}102882, 169234, KCP_{—}186048, KCP_{—}197775 and KCP_{—}16152 at the 5q35 locus; the presence of the haplotype is diagnostic of Type II diabetes or of a susceptibility to Type II diabetes. In particular embodiments, the presence of the G, G, T, C, G, G, A haplotype at rs1032856, KCP_RS888934, KCP_{—}93545, KCP_{—}102882, 169234, KCP_{—}186048 and KCP_{—}16152, or the presence of the G, G, T, C, G, G, C, A haplotype at rs1032856, KCP_RS888934, KCP_{—}93545, KCP_{—}102882, 169234, KCP_{—}186048, KCP_{—}197775 and KCP_{—}16152 is diagnostic of Type II diabetes or of a susceptibility to Type II diabetes.

Kits (e.g., reagent kits) useful in the methods of diagnosis comprise components useful in any of the methods described herein, including for example, hybridization probes or primers as described herein (e.g., labeled probes or primers), reagents for detection of labeled molecules, restriction enzymes (e.g., for RFLP analysis), allele-specific oligonucleotides, antibodies which bind to altered or to non-altered (native) KChIP1 polypeptide, means for amplification of nucleic acids comprising a KChIP1 nucleic acid, or means for analyzing the nucleic acid sequence of a KChIP1 nucleic acid or for analyzing the amino acid sequence of a KChIP1 polypeptide as described herein, etc. In one embodiment, the kit for diagnosing a Type II diabetes or a susceptibility to Type II diabetes can comprise primers for nucleic acid amplification of a region in the KChIP1 nucleic acid comprising an at-risk haplotype that is more frequently present in an individual having Type II diabetes or who is susceptible to Type II diabetes. The primers can be designed using portions of the nucleic acids flanking SNPs that are indicative of Type II diabetes. In a certain embodiment, the primers are designed to amplify regions of the KChIP1 gene associated with an at-risk haplotype for Type II diabetes, as shown in Table 10 and 13, or more particularly the haplotypes described in Tables 2, 4, 5, 14, 18 and 20-24.

Screening Assays and Agents Identified Thereby

The invention provides methods (also referred to herein as “screening assays”) for identifying the presence of a nucleotide that hybridizes to a nucleic acid of the invention, as well as for identifying the presence of a polypeptide encoded by a nucleic acid of the invention. In one embodiment, the presence (or absence) of a nucleic acid molecule of interest (e.g., a nucleic acid that has significant homology with a nucleic acid of the invention) in a sample can be assessed by contacting the sample with a nucleic acid comprising a nucleic acid of the invention (e.g., a nucleic acid having the sequence of one of SEQ ID NOs: 1, 114-258, or the complement thereof, or a nucleic acid encoding an amino acid having the sequence of one of SEQ ID NOs: 2, or a fragment or variant of such nucleic acids), under stringent conditions as described above, and then assessing the sample for the presence (or absence) of hybridization. In one embodiment, high stringency conditions are conditions appropriate for selective hybridization. In another embodiment, a sample containing the nucleic acid molecule of interest is contacted with a nucleic acid containing a contiguous nucleotide sequence (e.g., a primer or a probe as described above) that is at least partially complementary to a part of the nucleic acid molecule of interest (e.g., a KChIP1 nucleic acid), and the contacted sample is assessed for the presence or absence of hybridization. In another embodiment, the nucleic acid containing a contiguous nucleotide sequence is completely complementary to a part of the nucleic acid molecule of interest.

In any of these embodiments, all or a portion of the nucleic acid of interest can be subjected to amplification prior to performing the hybridization.

In another embodiment, the presence (or absence) of a polypeptide of interest, such as a polypeptide of the invention or a fragment or variant thereof, in a sample can be assessed by contacting the sample with an antibody that specifically hybridizes to the polypeptide of interest (e.g., an antibody such as those described above), and then assessing the sample for the presence (or absence) of binding of the antibody to the polypeptide of interest.

In another embodiment, the invention provides methods for identifying agents (e.g., fusion proteins, polypeptides, peptidomimetics, prodrugs, receptors, binding agents, antibodies, small molecules or other drugs, or ribozymes) which alter (e.g., increase or decrease) the activity of the polypeptides described herein, or which otherwise interact with the polypeptides herein. For example, such agents can be agents which bind to polypeptides described herein (e.g., KChIP1 binding agents); which have a stimulatory or inhibitory effect on, for example, activity of polypeptides of the invention; or which change (e.g., enhance or inhibit) the ability of the polypeptides of the invention to interact with KChIP1 binding agents (e.g., receptors or other binding agents); or which alter posttranslational processing of the KChIP1 polypeptide (e.g., agents that alter proteolytic processing to direct the polypeptide from where it is normally synthesized to another location in the cell, such as the cell surface; agents that alter proteolytic processing such that more polypeptide is released from the cell, etc.

In one embodiment, the invention provides assays for screening candidate or test agents that bind to or modulate the activity of polypeptides described herein (or biologically active portion(s) thereof), as well as agents identifiable by the assays. Test agents can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide libraries, while the other four approaches are applicable to polypeptide, non-peptide oligomer or small molecule libraries of compounds (Lam, K. S., Anticancer Drug Des. 12:145 (1997)).

In one embodiment, to identify agents which alter the activity of a KChIP1 polypeptide, a cell, cell lysate, or solution containing or expressing a KChIP1 polypeptide, or another splicing variant encoded by a KChIP1 gene (such as comprising a SNP as shown in Table 10 and/or 3), or a fragment or derivative thereof (as described above), can be contacted with an agent to be tested; alternatively, the polypeptide can be contacted directly with the agent to be tested. The level (amount) of KChIP1 activity is assessed (e.g., the level (amount) of KChIP1 activity is measured, either directly or indirectly), and is compared with the level of activity in a control (i.e., the level of activity of the KChIP1 polypeptide or active fragment or derivative thereof in the absence of the agent to be tested). If the level of the activity in the presence of the agent differs, by an amount that is statistically significant, from the level of the activity in the absence of the agent, then the agent is an agent that alters the activity of a KChIP1 polypeptide. An increase in the level of KChIP1 activity relative to a control, indicates that the agent is an agent that enhances (is an agonist of) KChIP1 activity. Similarly, a decrease in the level of KChIP1 activity relative to a control, indicates that the agent is an agent that inhibits (is an antagonist of) KChIP1 activity. In another embodiment, the level of activity of a KChIP1 polypeptide or derivative or fragment thereof in the presence of the agent to be tested, is compared with a control level that has previously been established. A level of the activity in the presence of the agent that differs from the control level by an amount that is statistically significant indicates that the agent alters KChIP1 activity.

The present invention also relates to an assay for identifying agents which alter the expression of a KChIP1 nucleic acid (e.g., antisense nucleic acids, fusion proteins, polypeptides, peptidomimetics, prodrugs, receptors, binding agents, antibodies, small molecules or other drugs, or ribozymes) which alter (e.g., increase or decrease) expression (e.g., transcription or translation) of the gene or which otherwise interact with the nucleic acids described herein, as well as agents identifiable by the assays. For example, a solution containing a nucleic acid encoding a KChIP1 polypeptide (e.g., a KChIP1 gene or nucleic acid) can be contacted with an agent to be tested. The solution can comprise, for example, cells containing the nucleic acid or cell lysate containing the nucleic acid; alternatively, the solution can be another solution that comprises elements necessary for transcription/translation of the nucleic acid. Cells not suspended in solution can also be employed, if desired. The level and/or pattern of KChIP1 expression (e.g., the level and/or pattern of mRNA or of protein expressed, such as the level and/or pattern of different splicing variants) is assessed, and is compared with the level and/or pattern of expression in a control (i.e., the level and/or pattern of the KChIP1 expression in the absence of the agent to be tested). If the level and/or pattern in the presence of the agent differs, by an amount or in a manner that is statistically significant, from the level and/or pattern in the absence of the agent, then the agent is an agent that alters the expression of a Type II diabetes gene. Enhancement of KChIP1 expression indicates that the agent is an agonist of KChIP1 activity. Similarly, inhibition of KChIP1 expression indicates that the agent is an antagonist of KChIP1 activity. In another embodiment, the level and/or pattern of KChIP1 polypeptide(s) (e.g., different splicing variants) in the presence of the agent to be tested, is compared with a control level and/or pattern that have previously been established. A level and/or pattern in the presence of the agent that differs from the control level and/or pattern by an amount or in a manner that is statistically significant indicates that the agent alters KChIP1 expression.

In another embodiment of the invention, agents which alter the expression of a KChIP1 nucleic acid or which otherwise interact with the nucleic acids described herein, can be identified using a cell, cell lysate, or solution containing a nucleic acid encoding the promoter region of the KChIP1 gene or nucleic acid operably linked to a reporter gene. After contact with an agent to be tested, the level of expression of the reporter gene (e.g., the level of mRNA or of protein expressed) is assessed, and is compared with the level of expression in a control (i.e., the level of the expression of the reporter gene in the absence of the agent to be tested). If the level in the presence of the agent differs, by an amount or in a manner that is statistically significant, from the level in the absence of the agent, then the agent is an agent that alters the expression of the KChIP1, as indicated by its ability to alter expression of a gene that is operably linked to the KChIP1 gene promoter. Enhancement of the expression of the reporter indicates that the agent is an agonist of KChIP1 activity. Similarly, inhibition of the expression of the reporter indicates that the agent is an antagonist of KChIP1 activity. In another embodiment, the level of expression of the reporter in the presence of the agent to be tested is compared with a control level that has previously been established. A level in the presence of the agent that differs from the control level by an amount or in a manner that is statistically significant indicates that the agent alters expression.

Agents which alter the amounts of different splicing variants encoded by a KChIP1 nucleic acid (e.g., an agent which enhances activity of a first splicing variant, and which inhibits activity of a second splicing variant), as well as agents which are agonists of activity of a first splicing variant and antagonists of activity of a second splicing variant, can easily be identified using these methods described above.

In other embodiments of the invention, assays can be used to assess the impact of a test agent on the activity of a polypeptide in relation to a KChIP1 binding agent. For example, a cell that expresses a compound that interacts with a KChIP1 polypeptide (herein referred to as a “KChIP1 binding agent”, which can be a polypeptide or other molecule that interacts with a KChIP1 polypeptide, such as a receptor) is contacted with a KChIP1 in the presence of a test agent, and the ability of the test agent to alter the interaction between the KChIP1 and the KChIP1 binding agent is determined. Alternatively, a cell lysate or a solution containing the KChIP1 binding agent, can be used. An agent which binds to the KChIP1 or the KChIP1 binding agent can alter the interaction by interfering with, or enhancing the ability of the KChIP1 to bind to, associate with, or otherwise interact with the KChIP1 binding agent. Determining the ability of the test agent to bind to a KChIP1 nucleic acid or a KChIP1 binding agent can be accomplished, for example, by coupling the test agent with a radioisotope or enzymatic label such that binding of the test agent to the polypeptide can be determined by detecting the labeled with ¹²⁵I, ³⁵S, ¹⁴C or ³H, either directly or indirectly, and the radioisotope detected by direct counting of radioemmission or by scintillation counting. Alternatively, test agents can be enzymatically labeled with, for example, horseradish peroxidase, alkaline phosphatase, or luciferase, and the enzymatic label detected by determination of conversion of an appropriate substrate to product. It is also within the scope of this invention to determine the ability of a test agent to interact with the polypeptide without the labeling of any of the interactants. For example, a microphysiometer can be used to detect the interaction of a test agent with a KChIP1 polypeptide or a KChIP1 binding agent without the labeling of either the test agent, KChIP1 polypeptide, or the KChIP1 binding agent. McConnell, H. M. et al., Science 257:1906-1912 (1992). As used herein, a “microphysiometer” (e.g., Cytosensor™) is an analytical instrument that measures the rate at which a cell acidifies its environment using a light-addressable potentiometric sensor (LAPS). Changes in this acidification rate can be used as an indicator of the interaction between ligand and polypeptide.

Thus, these receptors can be used to screen for compounds that are agonists or antagonists, for use in treating a susceptibility to a disease or condition associated with a KChIP1 gene or nucleic acid, or for studying a susceptibility to a disease or condition associated with a KChIP1 (e.g., Type II diabetes). Drugs could be designed to regulate KChIP1 activation that in turn can be used to regulate signaling pathways and transcription events of genes downstream.

In another embodiment of the invention, assays can be used to identify polypeptides that interact with one or more KChIP1 polypeptides, as described herein. For example, a yeast two-hybrid system such as that described by Fields and Song (Fields, S. and Song, O., Nature 340:245-246 (1989)) can be used to identify polypeptides that interact with one or more KChIP1 polypeptides. In such a yeast two-hybrid system, vectors are constructed based on the flexibility of a transcription factor that has two functional domains (a DNA binding domain and a transcription activation domain). If the two domains are separated but fused to two different proteins that interact with one another, transcriptional activation can be achieved, and transcription of specific markers (e.g., nutritional markers such as His and Ade, or color markers such as lacZ) can be used to identify the presence of interaction and transcriptional activation. For example, in the methods of the invention, a first vector is used which includes a nucleic acid encoding a DNA binding domain and also a KChIP1 polypeptide, splicing variant, or fragment or derivative thereof, and a second vector is used which includes a nucleic acid encoding a transcription activation domain and also a nucleic acid encoding a polypeptide which potentially may interact with the KChIP1 polypeptide, splicing variant, or fragment or derivative thereof (e.g., a KChIP1 polypeptide binding agent or receptor). Incubation of yeast containing the first vector and the second vector under appropriate conditions (e.g., mating conditions such as used in the Matchmaker™ system from Clontech (Palo Alto, Calif., USA)) allows identification of colonies that express the markers of interest. These colonies can be examined to identify the polypeptide(s) that interact with the KChIP1 polypeptide or fragment or derivative thereof. Such polypeptides may be useful as agents that alter the activity of expression of a KChIP1 polypeptide, as described above.

In more than one embodiment of the above assay methods of the present invention, it may be desirable to immobilize either the KChIP1 gene or nucleic acid, the KChIP1 polypeptide, the KChIP1 binding agent, or other components of the assay on a solid support, in order to facilitate separation of complexed from uncomplexed forms of one or both of the polypeptides, as well as to accommodate automation of the assay. Binding of a test agent to the polypeptide, or interaction of the polypeptide with a binding agent in the presence and absence of a test agent, can be accomplished in any vessel suitable for containing the reactants. Examples of such vessels include microtitre plates, test tubes, and micro-centrifuge tubes. In one embodiment, a fusion protein (e.g., a glutathione-S-transferase fusion protein) can be provided which adds a domain that allows a KChIP1 nucleic acid, KChIP1 polypeptide, or a KChIP1 binding agent to be bound to a matrix or other solid support.

In another embodiment, modulators of expression of nucleic acid molecules of the invention are identified in a method wherein a cell, cell lysate, or solution containing a KChIP1 nucleic acid is contacted with a test agent and the expression of appropriate mRNA or polypeptide (e.g., splicing variant(s)) in the cell, cell lysate, or solution, is determined. The level of expression of appropriate mRNA or polypeptide(s) in the presence of the test agent is compared to the level of expression of mRNA or polypeptide(s) in the absence of the test agent. The test agent can then be identified as a modulator of expression based on this comparison. For example, when expression of mRNA or polypeptide is greater (statistically significantly greater) in the presence of the test agent than in its absence, the test agent is identified as a stimulator or enhancer of the mRNA or polypeptide expression. Alternatively, when expression of the mRNA or polypeptide is less (statistically significantly less) in the presence of the test agent than in its absence, the test agent is identified as an inhibitor of the mRNA or polypeptide expression. The level of mRNA or polypeptide expression in the cells can be determined by methods described herein for detecting mRNA or polypeptide.

This invention further pertains to novel agents identified by the above-described screening assays. Accordingly, it is within the scope of this invention to further use an agent identified as described herein in an appropriate animal model. For example, an agent identified as described herein (e.g., a test agent that is a modulating agent, an antisense nucleic acid molecule, a specific antibody, or a polypeptide-binding agent) can be used in an animal model to determine the efficacy, toxicity, or side effects of treatment with such an agent. Alternatively, an agent identified as described herein can be used in an animal model to determine the mechanism of action of such an agent.

Furthermore, this invention pertains to uses of novel agents identified by the above-described screening assays for treatments as described herein. In addition, an agent identified as described herein can be used to alter activity of a polypeptide encoded by a KChIP1 nucleic acid, or to alter expression of a KChIP1 nucleic acid, by contacting the polypeptide or the nucleic acid (or contacting a cell comprising the polypeptide or the nucleic acid) with the agent identified as described herein.

Pharmaceutical Compositions

The present invention also pertains to pharmaceutical compositions comprising nucleic acids described herein, particularly nucleotides encoding the polypeptides described herein (e.g., a KChIP1 polypeptide); comprising polypeptides described herein and/or comprising other splicing variants encoded by a KChIP1 nucleic acid; and/or an agent that alters (e.g., enhances or inhibits) KChIP1 nucleic acid expression or KChIP1 polypeptide activity as described herein. For instance, a polypeptide, protein (e.g., a KChIP1 nucleic acid receptor), an agent that alters KChIP1 nucleic acid expression, or a KChIP1 binding agent or binding partner, fragment, fusion protein or pro-drug thereof, or a nucleotide or nucleic acid construct (vector) comprising a nucleotide of the present invention, or an agent that alters KChIP1 polypeptide activity, can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devises (“gene guns”) and slow release polymeric devices. The pharmaceutical compositions of this invention can also be administered as part of a combinatorial therapy with other agents.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The agents are administered in a therapeutically effective amount. The amount of agents which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms, and should be decided according to the judgment of a practitioner and each patient's circumstances. Effective doses may be extrapolated from dose-response curves derived from in vitro or animal model test systems.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use of sale for human administration. The pack or kit can be labeled with information regarding mode of administration, sequence of drug administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the agents can be separated, mixed together in any combination, present in a single vial or tablet. Agents assembled in a blister pack or other dispensing means is preferred. For the purpose of this invention, unit dosage is intended to mean a dosage that is dependent on the individual pharmacodynamics of each agent and administered in FDA approved dosages in standard time courses.

Methods of Therapy

The present invention also pertains to methods of treatment (prophylactic and/or therapeutic) for certain diseases and conditions associated with KChIP1. In particular, the invention relates to methods of treatment for Type II diabetes or a susceptibility to Type II diabetes, using a Type II diabetes therapeutic agent. A “Type II diabetes therapeutic agent” is an agent that alters (e.g., enhances or inhibits) KChIP1 polypeptide activity and/or KChIP1 nucleic acid expression, as described herein (e.g., a Type II diabetes nucleic acid agonist or antagonist). In certain embodiments, the Type II diabetes therapeutic agent alters activity and/or nucleic acid expression of KChIP1.

Type II diabetes therapeutic agents can alter KChIP1 polypeptide activity or nucleic acid expression by a variety of means, such as, for example, by providing additional KChIP1 polypeptide or by upregulating the transcription or translation of the KChIP1 nucleic acid; by altering posttranslational processing of the KChIP1 polypeptide; by altering transcription of KChIP1 splicing variants; or by interfering with KChIP1 polypeptide activity (e.g., by binding to a KChIP1 polypeptide), or by binding to another polypeptide that interacts with KChIP1, by altering (e.g., downregulating) the expression, transcription or translation of a KChIP1 nucleic acid, or by altering (e.g., agonizing or antagonizing) activity.

Representative Type II diabetes therapeutic agents include the following: nucleic acids or fragments or derivatives thereof described herein, particularly nucleotides encoding the polypeptides described herein and vectors comprising such nucleic acids (e.g., a gene, cDNA, and/or mRNA, such as a nucleic acid encoding a KChIP1 polypeptide or active fragment or derivative thereof, or an oligonucleotide; or a complement thereof, or fragments or derivatives thereof, and/or other splicing variants encoded by a Type II diabetes nucleic acid, or fragments or derivatives thereof); polypeptides described herein and/or splicing variants encoded by the KChIP1 nucleic acid or fragments or derivatives thereof; other polypeptides (e.g., KChIP1 receptors); KChIP1 binding agents; or agents that affect (e.g., increase or decrease) activity, antibodies, such as an antibody to an altered KChIP1 polypeptide, or an antibody to a non-altered KChIP1 polypeptide, or an antibody to a particular splicing variant encoded by a KChIP1 nucleic acid as described above; peptidomimetics; fusion proteins or prodrugs thereof; ribozymes; other small molecules; and other agents that alter (e.g., enhance or inhibit) expression of a KChIP1 nucleic acid, or that regulate transcription of KChIP1 splicing variants (e.g., agents that affect which splicing variants are expressed, or that affect the amount of each splicing variant that is expressed). More than one Type II diabetes therapeutic agent can be used concurrently, if desired.

A Type II diabetes nucleic acid therapeutic agent that is a nucleic acid is used in the treatment of Type II diabetes or in the treatment for a susceptibility to Type II diabetes. The term, “treatment” as used herein, refers not only to ameliorating symptoms associated with the disease or condition, but also preventing or delaying the onset of the disease or condition, and also lessening the severity or frequency of symptoms of the disease or condition. The therapy is designed to alter (e.g., inhibit or enhance), replace or supplement activity of a KChIP1 polypeptide in an individual. For example, a Type II diabetes therapeutic agent can be administered in order to upregulate or increase the expression or availability of the KChIP1 nucleic acid or of specific splicing variants of KChIP1 nucleic acid, or, conversely, to downregulate or decrease the expression or availability of the KChIP1 nucleic acid or specific splicing variants of the KChIP1 nucleic acid. Upregulation or increasing expression or availability of a native KChIP1 gene or nucleic acid or of a particular splicing variant could interfere with or compensate for the expression or activity of a defective gene or another splicing variant; downregulation or decreasing expression or availability of a native KChIP1 gene or of a particular splicing variant could minimize the expression or activity of a defective gene or the particular splicing variant and thereby minimize the impact of the defective gene or the particular splicing variant.

The Type II diabetes therapeutic agent(s) are administered in a therapeutically effective amount (i.e., an amount that is sufficient to treat the disease, such as by ameliorating symptoms associated with the disease, preventing or delaying the onset of the disease, and/or also lessening the severity or frequency of symptoms of the disease). The amount which will be therapeutically effective in the treatment of a particular individual's disorder or condition will depend on the symptoms and severity of the disease, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

In one embodiment, a nucleic acid of the invention (e.g., a nucleic acid encoding a KChIP1 polypeptide, such as one of SEQ ID NO: 1 or a complement thereof); or another nucleic acid that encodes a KChIP1 polypeptide or a splicing variant, derivative or fragment thereof (e.g., comprising any one or more of SEQ ID NO: 114-258), can be used, either alone or in a pharmaceutical composition as described above. For example, a KChIP1 gene or nucleic acid or a cDNA encoding a KChIP1 polypeptide, either by itself or included within a vector, can be introduced into cells (either in vitro or in vivo) such that the cells produce native KChIP1 polypeptide. If necessary, cells that have been transformed with the gene or cDNA or a vector comprising the gene, nucleic acid or cDNA can be introduced (or re-introduced) into an individual affected with the disease. Thus, cells which, in nature, lack native KChIP1 expression and activity, or have altered KChIP1 expression and activity, or have expression of a disease-associated KChIP1 splicing variant, can be engineered to express the KChIP1 polypeptide or an active fragment of the KChIP1 polypeptide (or a different variant of the KChIP1 polypeptide). In certain embodiments, nucleic acids encoding a KChIP1 polypeptide, or an active fragment or derivative thereof, can be introduced into an expression vector, such as a viral vector, and the vector can be introduced into appropriate cells in an animal. Other gene transfer systems, including viral and nonviral transfer systems, can be used. Alternatively, nonviral gene transfer methods, such as calcium phosphate coprecipitation, mechanical techniques (e.g., microinjection); membrane fusion-mediated transfer via liposomes; or direct DNA uptake, can also be used.

Alternatively, in another embodiment of the invention, a nucleic acid of the invention; a nucleic acid complementary to a nucleic acid of the invention; or a portion of such a nucleic acid (e.g., an oligonucleotide as described below), can be used in “antisense” therapy, in which a nucleic acid (e.g., an oligonucleotide) which specifically hybridizes to the mRNA and/or genomic DNA of a Type II diabetes gene is administered or generated in situ. The antisense nucleic acid that specifically hybridizes to the mRNA and/or DNA inhibits expression of the KChIP1 polypeptide, e.g., by inhibiting translation and/or transcription. Binding of the antisense nucleic acid can be by conventional base pair complementarity, or, for example, in the case of binding to DNA duplexes, through specific interaction in the major groove of the double helix.

An antisense construct of the present invention can be delivered, for example, as an expression plasmid as described above. When the plasmid is transcribed in the cell, it produces RNA that is complementary to a portion of the mRNA and/or DNA which encodes the KChIP1 polypeptide. Alternatively, the antisense construct can be an oligonucleotide probe that is generated ex vivo and introduced into cells; it then inhibits expression by hybridizing with the mRNA and/or genomic DNA of the polypeptide. In one embodiment, the oligonucleotide probes are modified oligonucleotides, which are resistant to endogenous nucleases, e.g., exonucleases and/or endonucleases, thereby rendering them stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy are also described, for example, by Van der Krol et al., (BioTechniques 6:958-976 (1988)); and Stein et al., (Cancer Res. 48:2659 2668 (1988)). With respect to antisense DNA, oligodeoxyribonucleotides derived from the translation initiation site are preferred.

To perform antisense therapy, oligonucleotides (mRNA, cDNA or DNA) are designed that are complementary to mRNA encoding the KChIP1. The antisense oligonucleotides bind to KChIP1 mRNA transcripts and prevent translation. Absolute complementarity, although preferred, is not required. A sequence “complementary” to a portion of an RNA, as referred to herein, indicates that a sequence has sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid, as described in detail above. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures.

The oligonucleotides used in antisense therapy can be DNA, RNA, or chimeric mixtures or derivatives or modified versions thereof, single-stranded or double-stranded. The oligonucleotides can be modified at the base moiety, sugar moiety, or phosphate backbone, for example, to improve stability of the molecule, hybridization, etc. The oligonucleotides can include other appended groups such as peptides (e.g. for targeting host cell receptors in vivo), or agents facilitating transport across the cell membrane (see, e.g., Letsinger et al., Proc. Natl. Acad. Sci. USA 86:6553-6556 (1989); Lemaitre et al., Proc. Natl. Acad. Sci. USA 84:648-652 (1987); PCT International Publication NO: WO 88/09810) or the blood-brain barrier (see, e.g., PCT International Publication NO: WO 89/10134), or hybridization-triggered cleavage agents (see, e.g., Krol et al., BioTechniques 6:958-976 (1988)) or intercalating agents. (See, e.g., Zon, Pharm. Res. 5:539-549 (1988)). To this end, the oligonucleotide may be conjugated to another molecule (e.g., a peptide, hybridization triggered cross-linking agent, transport agent, hybridization-triggered cleavage agent).

The antisense molecules are delivered to cells that express KChIP1 in vivo. A number of methods can be used for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically. Alternatively, in a preferred embodiment, a recombinant DNA construct is utilized in which the antisense oligonucleotide is placed under the control of a strong promoter (e.g., pol III or pol II). The use of such a construct to transfect target cells in the patient results in the transcription of sufficient amounts of single stranded RNAs that will form complementary base pairs with the endogenous KChIP1 transcripts and thereby prevent translation of the KChIP1 mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art and described above. For example, a plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systemically).

Endogenous KChIP1 polypeptide expression can also be reduced by inactivating or “knocking out” the gene, nucleic acid or its promoter using targeted homologous recombination (e.g., see Smithies et al., Nature 317:230-234 (1985); Thomas & Capecchi, Cell 51:503-512 (1987); Thompson et al., Cell 5:313-321 (1989)). For example, an altered, non-functional gene or nucleic acid (or a completely unrelated DNA sequence) flanked by DNA homologous to the endogenous gene or nucleic acid (either the coding regions or regulatory regions of the nucleic acid) can be used, with or without a selectable marker and/or a negative selectable marker, to transfect cells that express the gene or nucleic acid in vivo. Insertion of the DNA construct, via targeted homologous recombination, results in inactivation of the gene or nucleic acid. The recombinant DNA constructs can be directly administered or targeted to the required site in vivo using appropriate vectors, as described above. Alternatively, expression of non-altered genes or nucleic acids can be increased using a similar method: targeted homologous recombination can be used to insert a DNA construct comprising a non-altered functional gene or nucleic acid, e.g., a nucleic acid comprising one or more of SEQ ID NOs: 114-258 or the complement thereof, or a portion thereof, in place of an altered KChIP1 in the cell, as described above. In another embodiment, targeted homologous recombination can be used to insert a DNA construct comprising a nucleic acid that encodes a Type II diabetes polypeptide variant that differs from that present in the cell.

Alternatively, endogenous KChIP1 nucleic acid expression can be reduced by targeting deoxyribonucleotide sequences complementary to the regulatory region of a KChIP1 nucleic acid (i.e., the KChIP1 promoter and/or enhancers) to form triple helical structures that prevent transcription of the KChIP1 nucleic acid in target cells in the body. (See generally, Helene, C., Anticancer Drug Des., 6(6):569-84 (1991); Helene, C. et al., Ann. N.Y. Acad. Sci. 660:27-36 (1992); and Maher, L. J., Bioassays 14(12):807-15 (1992)). Likewise, the antisense constructs described herein, by antagonizing the normal biological activity of one of the KChIP1 proteins, can be used in the manipulation of tissue, e.g., tissue differentiation, both in vivo and for ex vivo tissue cultures. Furthermore, the anti-sense techniques (e.g., microinjection of antisense molecules, or transfection with plasmids whose transcripts are anti-sense with regard to a Type II diabetes gene mRNA or gene sequence) can be used to investigate the role of KChIP1 or the interaction of KChIP1 and its binding agents in developmental events, as well as the normal cellular function of KChIP1 or of the interaction of KChIP1 and its binding agents in adult tissue. Such techniques can be utilized in cell culture, but can also be used in the creation of transgenic animals.

In yet another embodiment of the invention, other Type II diabetes therapeutic agents as described herein can also be used in the treatment or prevention of a susceptibility to a disease or condition associated with a Type II diabetes gene. The therapeutic agents can be delivered in a composition, as described above, or by themselves. They can be administered systemically, or can be targeted to a particular tissue. The therapeutic agents can be produced by a variety of means, including chemical synthesis; recombinant production; in vivo production (e.g., a transgenic animal, such as U.S. Pat. No. 4,873,316 to Meade et al.), for example, and can be isolated using standard means such as those described herein.

A combination of any of the above methods of treatment (e.g., administration of non-altered polypeptide in conjunction with antisense therapy targeting altered mRNA of KChIP1; administration of a first splicing variant encoded by a KChIP1 nucleic acid in conjunction with antisense therapy targeting a second splicing encoded by a KChIP1 nucleic acid) can also be used.

The present invention is now illustrated by the following Exemplification, which is not intended to be limiting in any way. All references cited herein are incorporated by reference in their entirety.

### EXEMPLIFICATION

The study was done in collaboration with the Icelandic Heart Association, who provided an encrypted list of 1350 diabetic patients. In 1967-1991, the Heart Association started a study of cardiovascular disease and its complications. Measurements of blood sugar were included in a thorough check-up of the participants which results led to many individuals being diagnosed with diabetes. The list of participants is an unbiased sample of about a third of the Icelandic nation. Individuals diagnosed in the years following 1991 were either diagnosed at the Icelandic Heart Association or at one of two major hospitals in Reykjavik, Iceland.

All participants in the Type II diabetes study visited the Icelandic Heart Association where each answered a questionnaire, had blood drawn, a blood sugar assessment, and measurements taken. Height (m) and weight (kg) were measured to calculate the body mass index. In serum, the fasting blood glucose and triglyceride levels were measured as well. Diagnoses of Type II diabetes were based on the diagnostic criteria set by the World Health Organization (1999). All patients with fasting glucose above 7 mM were diagnosed as having Type II diabetes and individuals with fasting blood sugar between 6.1-6.9 mM were diagnosed with impaired fasting glucose. If the participants had no prior history of diabetes, they were requested to come in for another test to have their diagnosis confirmed. All individuals on diabetic medication were classified as Type II. The questionnaire included questions regarding age at diagnosis and type of medication. All patients were requested to bring two relatives who's DNA was used to confirm the genetotypes of the patients.

Since the patients had participated in a study that was conducted between 1967-1991 a considerable time had passed, in some instances, since they had visited the Heart Association. Therefore, all the patients were required to have another fasting blood glucose test to check on their blood sugar level at the time of participation in the study. Thus, all patients were labeled unconfirmed, meaning that results of blood glucose levels were pending, for this particular study. A label of confirmed diabetic was given to the patient when the measurements were received. Linkage analyses were done with confirmed patients and unconfirmed patients were included only if they were close relatives of a confirmed index patient. The initial list of patients included 1350 Type II diabetics, but during this study new patients were diagnosed who were relatives of the index patients. All participants with no previous history of diabetes but with elevated fasting glucose were diagnosed according to the WHO criteria as described above. At present date, 1406 Type II diabetics and 266 patients with impaired fasting glucose have participated in the study, together with 3972 of their close relatives.

This study was approved by the Data Protection Commission of Iceland and the National Bioethics Committee of Iceland. All patients and their relatives who participated in the Study gave informed consents.

Outline of the Study

This particular genetic study, which has the aim of identifying a genetic variant or a gene that may contribute to type II diabetes by using a positional cloning approach, can be divided into three steps:
i. Genome-wide linkage study, where excess allele sharing among related type II diabetics is used to identify a chromosomal segment, typically 2-8 Megabases long, that may harbor a disease susceptibility gene/genes.
ii. Locus-wide association study, where a high-density of microsatellite markers is typed in a large patient and control cohort. By comparing the frequencies of individual alleles or haplotypes between the two cohorts, the location of the putative disease gene/genes is narrowed down to a few hundred kilobases.
iii. Candidate gene assessment, where additional microsatellites and/or SNPs are typed in all genes that are identified within the smaller candidate region and further association analysis is used to identify which of the genes shows strong association to the disease.
   Linkage Analysis
   Pedigree Construction

For the linkage analysis, blood samples were obtained from 964 Type II diabetics and 203 individuals with impaired fasting glucose. The patients were clustered into families such that each patient is related to (within and including six meiotic events) at least one other patient. In this manner, 772 patients fell into families—705 Type II diabetics and 67 with impaired fasting glucose. The confirmed Type II patients were treated as probands and clustered into families that each proband is related to, within and including six meiotic events. The other patients, unconfirmed Type II and IFG patients, were added to the families if they were related to a proband within and including three meiotic events. The rational behind this was to include as many patients as possible in the study. Impaired fasting glucose is an immediate diagnosis, and we assumed that the more closely related these patients are to the confirmed diabetics, the likelier they are to have or to develop the disease.

The families were checked for relationship errors by comparing the identity-by state (IBS) distribution for the set of 906 markers, for each pair of related and genotyped individuals, to a reference distribution corresponding to the particular degree of relatedness. The reference distributions were constructed from a large subset of the Icelandic population. Individuals were excluded from the study if their relationship with the rest of the family was inconsistent with the relationship specified in the geneology database.

The remaining material that was available for the study was the following: 763 now confirmed Type II patients in 227 families together with 764 genotyped relatives. Of the patients, 667 were confirmed Type II patients, 35 unconfirmed Type II patients, 52 confirmed patients with impaired fasting glucose (IFG) and 9 unconfirmed patients with IFG.

Stratification of the Patient Material

The patients were classified into two sub-phenotypes based on their BMI: non-obese Type II diabetes are patients who have BMI less than 30, and obese Type II diabetes are patients who have BMI at or above 30. The reason for fractionating the diabetics into non-obese and obese groups is that other factors may be influencing the pathogenesis of disease in these two groups. Obesity alone could be contributing to the diabetic phenotype. Therefore, this factor was separated. Obesity is most likely due to a combination of environmental and genetic factors. This fractionation into non-obese and obese diabetics practically separates the material into two halves; 60% of the patients are in the non-obese category (20% with BMI below 25 (lean) and 40% with BMI between 25-30 (overweight)), and 40% of the patients are in the obese category (BMI above 30).

An affected-only linkage analysis for each of those sub-phenotypes was performed, using the same set of families as above, but classifying patients not belonging to the particular sub-group as having an unknown disease status. Restricted to a particular sub-phenotype, some families no longer contain a pair of related patients classified as affecteds and hence do not contribute in the linkage analysis. Such families were excluded from the analysis of the particular sub-phenotype. The number of patients and families used in the linkage analysis is summarized in Table 1 below.

Table 1: The number of patients and families that contribute to the genome-wide linkage scan, both when all the patients are used, and when the analysis is restricted to obese or non-obese diabetic patients, respectively.

TABLE 1
ts6Phenotype and Patients

| | | NO: of families | NO: of patients |
|---|---|---|---|
| | Total Number | contributing to | contributing to |
| Phenotype | of Patients | the analysis | the analysis |
| All diabetics | 763 | 227 | 763 |
| Obese | 296 | 92 | 219 |
| Non-obese | 467 | 154 | 413 |

Genome Wide Scan

A genome wide scan was performed on 772 patients and their relatives. Nine patients were excluded due to inheritance errors so the linkage analysis was performed with 763 patients and 764 relatives. The procedure was as described in Gretarsdóttir, et al., Am J Hum Genet., 70(3):593-603 (2002). In short, the DNA was genotyped with a framework marker set of 906 microsatellite markers with an average resolution of 4 cM. Alleles were called automatically with the TrueAllele program (Cybergenetics, Co., Pittsburgh, Pa.), and the program DecodeGT (deCODE genetics, ehf., Iceland), was used to fractionate according to quality and edit the called genotypes (Palsson, B., et al., Genome Res., 9(10):1002-1012 (1999)). The population allele frequencies for the markers were constructed from a cohort of more than 30,000 Icelanders that have participated in genome-wide studies of various disease projects at deCODE genetics. Additional markers were genotyped within the locus on chromosome 5q, where we observed the strongest linkage signal, to increase the information on identity by descent (IBD) sharing within the families. For those markers, at least 180 Icelandic controls were genotyped to derive the population allele frequencies.

The additional microsatellite markers that were genotyped within the locus were either publicly available or designed at deCODE genetics; those markers are indicated with a DG designation. Repeats within the DNA sequence were identified that allowed us to choose or design primers that were evenly spaced across the locus. The identification of the repeats and location with respect to other markers was based on the work of the physical mapping team at deCODE genetics.

For the markers used in the genomewide scan, the genetic positions were taken from the recently published high-resolution genetic map (HRGM), constructed at deCODE genetics (Kong A., et al., Nat Genet., 31: 241-247 (2002)). The genetic position of the additional markers are either taken from the HRGM, when available, or by applying the same genetic mapping methods as were used in constructing the HRGM map to the family material genotyped for this particular linkage study.

Statistical Methods for Linkage Analysis

The linkage analysis is done using the software Allegro (Gudbjartsson et al., Nat. Genet. 25:12-3, (2000)) that determines the statistical significance of excess sharing among related patients by applying non-parametric affected-only allele-sharing methods (without any particular disease inheritance model being specified). Allegro, a linkage program developed at deCODE genetics, calculates LOD scores based on multipoint calculations. Our baseline linkage analysis uses the Sₚₐᵢᵣₛ scoring function (Whittemore, A. S. and Halpern, J., Biometrics 50:118-27 (1994); Kruglyak L, et al., Am J Hum Genet 58:1347-63, (1996)), the exponential allele-sharing model (Kong, A. and Cox, N. J., Am. J. Hum. Genet., 61:1179 (1997)), and a family weighting scheme which is halfway on a log scale between weighting each affected pair equally and weighting each family equally. In the analysis, all genotyped individuals who are not affected are treated as “unknown”. Because of concern with small sample behavior, we usually compute corresponding P-values in two different ways for comparison. The first P-value is computed based on large sample theory; Zₗᵣ=√(2 logₑ(10) LOD) and is approximately distributed as a standard normal distribution under the null hypothesis of no linkage. A second P-value is computed by comparing the observed LOD score to its complete data sampling distribution under the null hypothesis. When a data set consists of more than a handful of families, these two P-values tend to be very similar.

All suggestive loci with LOD scores greater than 2 are followed up with some extra markers to increase the information on the IBD-sharing within the families and to decrease the chance that a LOD score represents a false-positive linkage. The information measure we use was defined by Nicolae (D. L. Nicolae, Thesis, University of Chicago (1999)) and is a part of the Allegro program output. This measure is closely related to a classical measure of information as previously described by Dempster et al. (Dempster, A. P., et al., J. R. Statist. Soc. B, 39:1 (1977)); the information equals zero if the marker genotypes are completely uninformative and equals one if the genotypes determine the exact amount of allele sharing by descent among the affected relatives. Using the framework marker set with average marker spacing of 4 cM typically results in information content of about 0.7 in the families used in our linkage analysis. Increasing the marker density to one marker every centimorgan usually increases the information content above 0.85.

Results

The results of the genome-wide linkage analysis with the framework marker set are shown in FIG. 4 which depicts the allele-sharing LOD-score versus the genetic distance from the p-terminus in centimorgan (cM) for each of the 23 chromosomes. The analysis was performed with the three phenotypes: all Type II diabetics (solid lines), non-obese diabetics (dashed lines) and obese diabetics (dotted lines). A LOD-score of 1.84 is observed on chromosome 5q34-q35.2 with the framework marker set when we use all Type II diabetics in the analysis. When the linkage analysis is restricted to non-obese diabetics, this LOD-score increases to 2.81. The obese diabetics do not show linkage in this region.

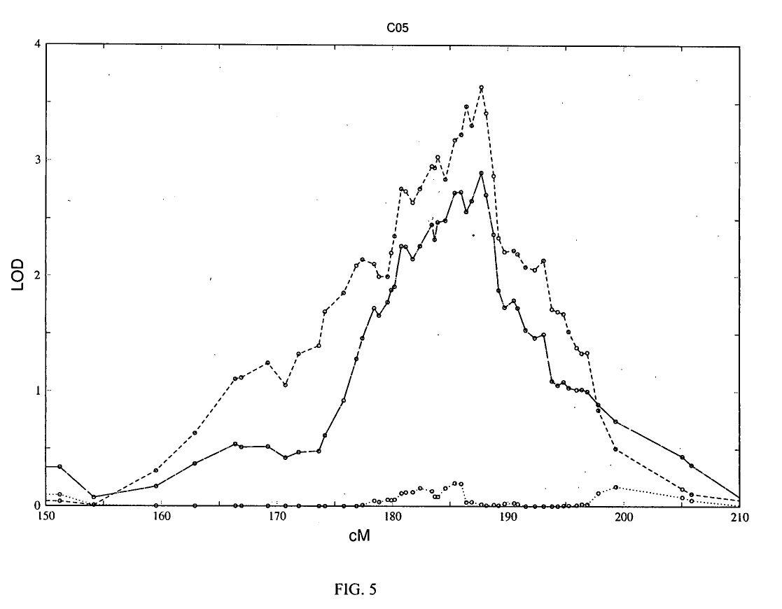

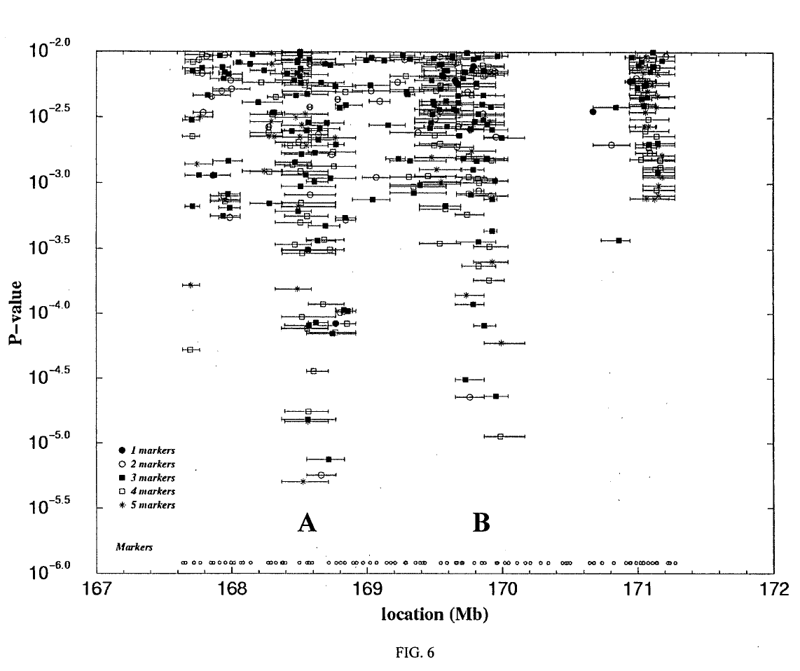

Additional markers were genotyped in this area to increase the information content and to confirm the linkage. The information on the IBD-sharing at this locus was about 78% with the framework marker set. In order to increase the information content, another 38 microsatellite markers were genotyped within a 40 cM region that includes the observed signal. Repeating the linkage analysis including the additional markers increased the LOD-score to 3.64 (P-value=3.18×10⁻⁵) for the non-obese diabetics. For all patients, the peak LOD-score increased to 2.9 (P-value=1.22×10⁻⁴). This is shown in FIG. 5.

The peak of the LOD-score is centered on marker D5S625 and the region determined by a drop of one in the LOD is from marker DG5S5 to marker D5S429, centromeric and telomeric respectively. The one-LOD-drop is about 9 cM and estimated to be about 3.5 Mb. This 1-LOD-drop roughly corresponds to the 80-90% confidence interval for the location of a putative disease associated gene.

Locus-Wide Association Study

Genotyping to Narrow Down the Region of Linkage

In order to narrow down the region of interest, the linkage analysis is followed by a comprehensive association study of the 1-LOD-drop. This is necessary as the linkage analysis has limited resolution; it compares sharing among closely related individuals that share on average large chromosomal segments. For the association analysis, we identified a large number of additional microsatellite markers located in the 1-LOD-drop and typed those markers in both our patient cohort and in a large number of unrelated controls randomly selected from the Icelandic population.

Sixty-seven markers were identified and typed in the 1-LOD-drop in addition to the 17 markers already typed and used in the linkage analysis (locus-wide association micorsatellites; Table 6). The new polymorphic repeats (dinucleotide or trinucleotide repeats) were identified with the Sputnik program. We subtracted the smaller allele of CEPH sample 1347-02 (CEPH genomics repository) from the alleles of the microsatellites and used it as a reference. A total of 84 markers were available for the association analysis, i.e., an average density of one marker every 42 kb or one marker every 0.107 cM. All those markers were typed for 590 non-obese diabetics and 477 unrelated controls.

Statistical Methods for Association and Haplotype Analysis

For single marker association to the disease, Fisher exact test was used to calculate a two-sided P-value for each individual allele. When presenting the results, we use allelic frequencies rather than carrier frequencies for microsatellites, SNPs and haplotypes. Haplotype analyses are performed using a computer program we developed at deCODE called NEMO (NEsted MOdels) (Gretarsdóttir, et al., Nat Genet. 2003 October; 35(2):131-8). NEMO was used both to study marker-marker association and to calculate linkage disequilibrium (LD) between markers, and for case-control haplotype analysis. With NEMO, haplotype frequencies are estimated by maximum likelihood and the differences between patients and controls are tested using a generalized likelihood ratio test. The maximum likelihood estimates, likelihood ratios and P-values are computed with the aid of the EM-algorithm directly for the observed data, and hence the loss of information due to the uncertainty with phase and missing genotypes is automatically captured by the likelihood ratios, and under most situations, large sample theory can be used to reliably determine statistical significance. The relative risk (RR) of an allele or a haplotype, i.e., the risk of an allele compared to all other alleles of the same marker, is calculated assuming the multiplicative model (Terwilliger, J. D. & Ott, J. A haplotype-based ‘haplotype relative risk’ approach to detecting allelic associations. Hum. Hered. 42, 337-46 (1992) and Falk, C. T. & Rubinstein, P. Haplotype relative risks: an easy reliable way to construct a proper control sample for risk calculations. Ann. Hum. Genet. 51 (Pt 3), 227-33 (1987)), together with the population attributable risk (PAR).

In the haplotype analysis, it may be useful to group haplotypes together and test the group as a whole for association to the disease. This is possible to do with NEMO. A model is defined by a partition of the set of all possible haplotypes, where haplotypes in the same group are assumed to confer the same risk while haplotypes in different groups can confer different risks. A null hypothesis and an alternative hypothesis are said to be nested when the latter corresponds to a finer partition than the former. NEMO provides complete flexibility in the partition of the haplotype space. In this way, it is possible to test multiple haplotypes jointly for association and to test if different at-risk haplotypes confer different risk. As a measure of LD, we use two standard definitions of LD, D′ and R² (Lewontin, R., Genetics, 49:49-67 (1964) and Hill, W. G. and A. Robertson, Theor. Appl. Genet., 22:226-231 (1968)) as they provide complementary information on the amount of LD. For the purpose of estimating D′ and R², the frequencies of all two-marker allele combinations are estimated using maximum likelihood methods and the deviation from linkage disequilibrium is evaluated using a likelihood ratio test. The standard definitions of D′ and R² are extended to include microsatellites by averaging over the values for all possible allele combinations of the two markers weighted by the marginal allele probabilities.

The number of possible haplotypes that can be constructed out of the dense set of markers genotyped in the 1-LOD-drop is very large and even though the number of haplotypes that are actually observed in the patient and control cohort is much smaller, testing all those haplotypes for association to the disease is a formidable task Note that we do not restrict our analysis to haplotypes constructed from a set of consecutive markers, as some markers may be very mutable and might split up an otherwise well conserved haplotype constructed out of surrounding markers.

The approach for identifying those haplotypes in the candidate region that show strongest association to the disease is two-fold. First, the haplotypes are restricted and tested to span a sub-region small enough that the included markers may be expected to be in substantial LD. In this study, only haplotypes that span less than 300 kb are considered. Second, an iterative procedure that gradually builds up the most significant haplotypes is applied. Starting with haplotypes constructed out of 3 markers, those haplotypes are selected that show strong association to the disease, other nearby markers to those haplotypes and repeat the association test are added. By iterating this procedure, identification of those haplotypes that show strongest association to the disease is expected.

Results

For the association analysis, 590 non-obese Icelandic Type II diabetes patients and 477 unrelated population controls using a total of 84 microsatellite markers were genotyped. These markers are distributed evenly across a region of approximately 3.5 Mb. The region is centered on our linkage peak and corresponds to the 1-LOD-drop. Then, the procedure described above is appliced and single-markers and haplotypes consisting of up to 5 markers that showed association to the disease are searched. The result is summarized in FIG. 6. In FIG. 6, the location of a marker or a haplotype on the horizontal axis and the corresponding P-value from the associaton test on the vertical axis is shown. This is shown for all haplotypes tested that have a P-value less than 0.01. The horizontal bars indicated the size of the corresponding haplotypes and the location of all markers is shown at the bottom of the figure. All locations are in Mb and refer to the NCBI Build33.

A series of correlated haplotypes that show strong association for non-obese diabetics in two locations within the 1-LOD-drop were observed. Those are denoted regions A (168.37-168.83 Mb) and B (169.70-170.17 Mb), and Table 10 lists the most significant haplotype in each of these regions. For each haplotype, the table includes a two-sided single-test P-value for association, calculated using NEMO, the corresponding relative risk, the estimated frequency of the haplotype in the patient and the control cohorts, the region the haplotype spans, and the markers and alleles (in bold) that define the haplotype.

Note, however, that some of the haplotypes listed within each of the two regions are very correlated and should be considered as a single observation of association to the disease. This is demonstrated for region B in Table 3, which lists the pairwise correlation, both D′ and R², between the haplotypes. Based on the correlation, we observe that haplotypes B2 and B4 are strongly correlated and should be considered as a single observation of association to this region. Likewise, haplotypes B1 and B5 are strongly correlated. However, haplotypes B1, B2 and B3 are all weakly correlated with each other; and in fact, B1 and B2 are mutually exclusive, i.e., never appear jointly on the same chromosome. These three haplotypes hence constitute three almost independent observations of association to non-obese diabetes of this region within the locus. It is possible to test haplotypes B1, B2 and B3 together as a group for association to non-obese diabetes. This test yields a P-value=8.5×10⁻⁸ with a corresponding relative risk of 5.2, a population attributable risk of 13.9%, and an allelic frequency of 0.089 and 0.018 in the patient and the control cohorts, respectively.

TABLE 2
Table 2: Haplotypes within the 1-LOD-drop that show the strongest association to non-obese
diabetes. For each haplotype, it is shown (i) a two-sided P-value for a single test
of association to non-obese diabetes, (ii) the corresponding relative risk (RR), (iii)
the estimated allelic frequency of the haplotype in the patient and the control cohort,
(iv) the span of the haplotype (refering to NCBI 33) and (v) the alleles (in bold) and
markers that define the haplotype. The haplotypes are separated into two groups, A and
B, corresponding to two different regions within the 1-LOD-drop.

| | | | | | Span | |
|---|---|---|---|---|---|---|
| | P-value | RR | Aff. frq | Ctrl. frq | (Mb) | Haplotype |
| A1 | 0.000005 | >10 | 0.033 | 0.000 | 168.37- | 0 DG5S879 4 DG5S881 −4 D5S2075 |
| | | | | | 168.72 | 0 DG5S883 4 DG5S38 |
| A2 | 0.000006 | 3.81 | 0.053 | 0.015 | 168.55- | 4 DG5S1058 −6 DG5S37 |
| | | | | | 168.77 | |
| A3 | 0.000008 | 3.64 | 0.054 | 0.015 | 168.55- | 4 DG5S1058 −6 DG5S37 0 |
| | | | | | 168.83 | DG5S101 |
| A4 | 0.000015 | 6.18 | 0.046 | 0.008 | 168.40- | 4 DG5S881 4 DG5S1058 −4 |
| | | | | | 168.72 | D5S2075 0 DG5S883 4 DG5S38 |
| A5 | 0.000015 | 4.42 | 0.047 | 0.011 | 168.37- | 0 DG5S879 4 DG5S1058 −6 |
| | | | | | 168.77 | DG5S37 |
| A6 | 0.000018 | 6.94 | 0.045 | 0.007 | 168.40- | 4 DG5S881 −4 D5S2075 0 DG5S883 |
| | | | | | 168.72 | 4 DG5S38 |
| B1 | 0.000011 | >10 | 0.039 | 0.000 | 169.87- | 0 DG5S953 0 DG5S955 0 DG5S13 5 |
| | | | | | 170.17 | DG5S959 |
| B2 | 0.000023 | >10 | 0.034 | 0.000 | 169.65- | 27 DG5S888 0 DG5S953 |
| | | | | | 169.87 | |
| B3 | 0.000023 | 5.26 | 0.049 | 0.010 | 169.87- | 0 DG5S953 0 DG5S955 4 DG5S124 |
| | | | | | 170.04 | |
| B4 | 0.000031 | >10 | 0.034 | 0.000 | 169.65- | 27 DG5S888 0 DG5S44 0 DG5S953 |
| | | | | | 169.87 | |
| B5 | 0.000060 | >10 | 0.034 | 0.000 | 169.87- | 0 DG5S953 0 DG5S955 0 DG5S13 0 |
| | | | | | 170.17 | DG5S123 5 DG5S959 |

TABLE 3
Table 3: Pairwise correlation between the five haplotypes in
the B-region that show the strongest association to non-obese
diabetes. Estimates of D′ are shown in the upper right corner,
and estimates of R² are shown the the lower left corner. The
haplotypes are labelled B1, . . . , B5 as in Table 2.

D′

| R2 | B1 | B2 | B3 | B4 | B5 |
|---|---|---|---|---|---|
| B1 | — | 0 | 0 | 0 | 1 |
| B2 | 0 | — | 0.4 | 1 | 0 |
| B3 | 0 | 0.1 | — | 0.35 | 0 |
| B4 | 0 | 0.96 | 0.7 | — | 0 |
| B5 | 0.92 | 0 | 0 | 0 | — |

Investigation of Region B
Genes in Region B

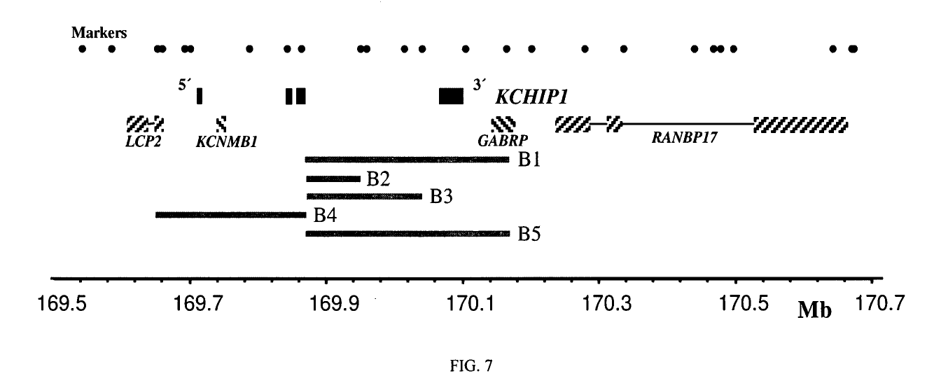

All genes in and around region B (UCSC) were then identified. In the region defined by the five most significant haplotypes, 169.70-170.17 Mb, there are four genes, LCP2 (lymphocyte cytosolic protein 2), KCNMB1 (potassium large conductance calcium-activated channel, subfamily M, beta member 1), KChIP1 (Kv channel interacting protein 1) and GABRP (gamma-aminobutyric acid (GABA) A receptor, pi). Of those genes, KChIP1 is by far the largest, stretching from 169.7 to 170.1 MB, or almost the entire span of the observed haplotype association. The other three genes are small. In addition, there is a big gene, RANBP17 (RAN binding protein 17), just telomeric of the location of the observed association signal. The relative location of all the genes is shown in FIG. 7, which shows the location of the exons of KCHIP1 as solid bars, and the location of the other genes as shaded boxes. In addition, FIG. 7 shows the location of the microsatellites (filled boxes) that we have typed in this region and the location of the at-risk haplotypes B1, . . . , B5 (gray horizontal lines).

Description of New Splice Variants of KChIP1 Identified by RACE and PCR

The published sequence for KChIP1 comprises exons 1 to 8. New exons belonging to the KChIP1 gene and four different splice variants were discovered by performing RACE or PCR (primers within the exons) using as template human Marathon cDNA and cDNA prepared from rat pancreatic INS1 beta cells. In all, 6 new exons located in the 5′ region of the gene were discovered. An alternative exon 1 was found and called exon 1a. Here, the published sequence is labelled for exon 1 with a “b” to distinguish it from the alternative exon 1, exon 1a. Four exons are called UTR 1, UTR 2, UTR 3 and UTR 4, or untranslated region 1-4, because they lie upstream of exon 1b and they are not translated. The last exon to be identified is called Ins-r, or insert rodent, because it was known to be present in mouse and rat, and has recently been demonstrated by others to be present in humans as well (Boland et al., Am. J. Physiol. Cell Physiol., 285, C161-170. (2003)). See nucleotide sequences of the new exons below, as well as their location in the genomic sequence of NCBI build 33. Even if not mentioned, all new variants of KChIP1 found and described below include exons 2-8 of the published sequence.

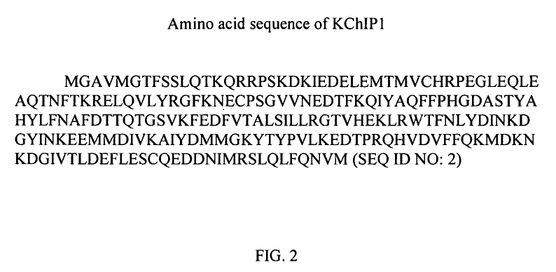

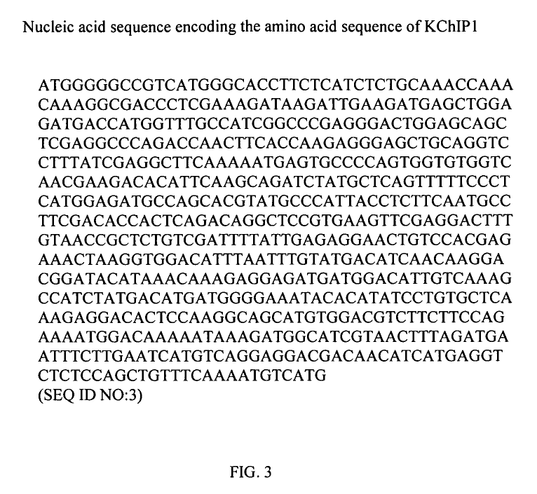

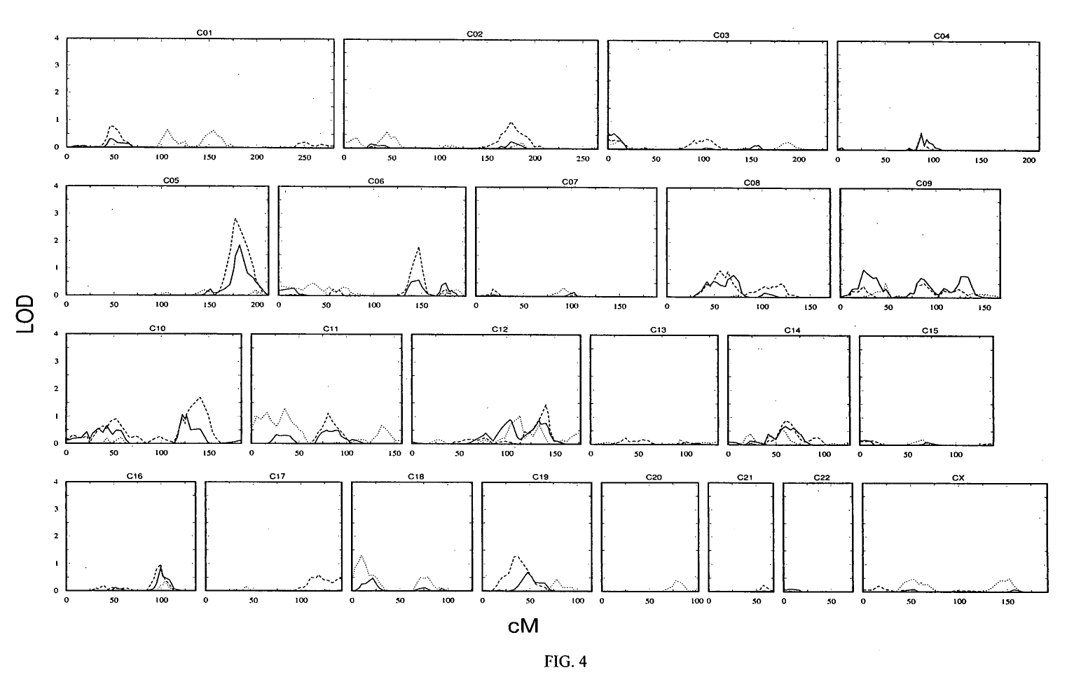

Splice variant 1 consists of exon 1a, UTR1, UTR2, UTR3, UTR4 and exon 1b. Exon 1a is untranslated and the resulting protein is identical in amino acid sequence to KChIP1 described by An et al. (Nature 430, 553-556 (2000), see also FIG. 2). This variant was observed in human heart and testis and the rat INS1 cell line.

Splice variant 2 consists of exon 1b and the Ins-r exon giving rise to a protein that is identical in amino acid sequence to KChIP1 described by Boland et al. This variant was observed in human brain, heart, pancreas and the rat INS1 cell line.

Splice variant 3 consists of exon 1a and is identical in nucleotide sequence to AL538404, an EST in NCBI. The amino acid sequence of the N-terminus coded by exon 1a is unique (see sequence below) but the amino acid sequence coded by exons 2-8 is that of the published sequence. This variant was observed in human brain, heart, pancreas, skeletal muscle, adipose tissue, liver, hypothalamus, small intestine, testis and the rat INS1 cell line.

Splice variant 4 consists of exons 1a and UTR1, which would result in a protein translated from exons 2-8. The second methionine in exon 2 has a Kozak sequence. This variant was observed in human heart.

The nucleotide sequences of the new exons are as follows (the genomic locations given are from NCBI build 33, see also Table 8):

| | |
|---|---|
| Exon 1a: 169716298-169716511 (Build 33) | |
| GGCTTCAGGGGTGCATCCGTCACTCAGGGTTCATTGACCCAGGCAGGCTCCAAGT | (SEQ ID NO: 4) |
| TCCTGGGGTGCACAAGGTGGGCACTGTCCCTTCTGGGTGCTGACAGCAGAGCCTG | |
| GCTCCCCTCCGCCACCATGAGCGGCTGCTCCAAAAGATGCAAGCTTGGGTTCGTG | |
| AAATTTGCCCAGACCATCTTTAAGCTCATCACTGGGACCCTCAGCAAAG | |
| UTR 1: 169848417-169848523 (Build 33) | |
| ACTCAGCATCATCAAGACTGGAGGGACAGAGCATTTGAATCATCAGACGCTGGGC | (SEQ ID NO: 5) |
| CAGACGTCACCCCACGCGTTTTCTCATTTTATC GTCCTAAGAAGCCCAGAAG | |
| UTR 2: 169861083-169861154 (Build 33) | |
| CCTGAATGCAATTTGCAATGAGGAGATGATTTGATTTTCTTCAGCCCTAGACCTCC | (SEQ ID NO: 6) |
| AGCTTCCTGAGAGCAG | |
| UTR 3: 169864589-169864679 (Build 33) | |
| GGGTTCCCCAGGAGACCACGACAGAGGCCTGGAACCCAAGTTCTAATCCCACATC | (SEQ ID NO: 7) |
| CTGGCTGGGCAACTTCAGGCAAATTTCTAACACAAG | |
| UTR 4: 169867066-169867173 (Build 33) | |
| GGTAGGGGAGGGGCCGGGCCCGGGGTCCCAACTCGCACTCAAGTCTTCGCTGCCA | (SEQ ID NO: 8) |
| TGGGGGCCGTCATGGGCACCTTCTCATCTCTGCaAACCAAACAAAGGCGACCC | |
| Ins-r 170075401-170075433 | |
| ACATCGCCTGGTGGTATTACCAGTATCAGAGAG | (SEQ ID NO: 9) |

The nucleotide sequence derived from splice variant 4 (KChIP1.4) with the ATG and a Kozak sequence ((G/ANNATGG) underlined is as follows:

| | |
|---|---|
| ATAAGATTGAAGATGAGCTGGAGATGACCATGGTTTGCCATCGGCCCGAGGGACT | (SEQ ID NO: 10) |
| GGAGCAGCTCGAGGCCCAGACCAACTTCACCAAGAGGGAGCTGCAGGTCCTTTAT | |
| CGAGGCTTCAAAAATGAGTGCCCCAGTGGTGTGGTCAACGAAGACACATTCAAGC | |
| AGATCTATGCTCAGTTTTTCCCTCATGGAGATGCCAGCACGTATGCCCATTACCTC | |
| TTCAATGCCTTCGACACCACTCAGACAGGCTCCGTGAAGTTCGAGGACTTTGTAAC | |
| CGCTCTGTCGATTTTATTGAGAGGAACTGTCCACGAGAAACTAAGGTGGACATTT | |
| AATTTGTATGACATCAACAAGGACGGATACATAAACAAAGAGGAGATGATGGAC | |
| ATTGTCAAAGCCATCTATGACATGATGGGGAAATACACATATCCTGTGCTCAAAG | |
| AGGACACTCCAAGGCAGCATGTGGACGTCTTCTTCCAGAAAATGGACAAAAATAA | |
| AGATGGCATCGTAACTTTAGATGAATTTCTTGAATCATGTCAGGAGGACGACAAC | |
| ATCATGAGGTCTCTCCAGCTGTTTCAAAATGTCATGTAACTGGTGACACTCAGCCA | |
| TTCAGCTCTCAGAGACATTGTACTAAACAACCACCTTAACACCCTGATCTGCCCTT | |
| GTTCTGATTTTACACACCAACTCTTGGGACAGAAACACCTTTTACACTTTGGAAGA | |
| ATTCTCTGCTGAAGACTTTCTATGGAACCCAGCATCATGTGGCTCAGTCTCTGATT | |
| GCCAACTCTTCCYCTTTCTTCTTCTTGAGAGAGA | |

The protein sequences resulting from the splice variants are as follows:

KChIP1.3

(The amino acid sequence derived from splice variant 3 (KChIP1.3), the underlined amino acids are coded by exon 1a.)

| | |
|---|---|
| MSGCSKRCKLGFVKFAQTIFKLITGTLSKDKIEDELEMTMVCHRPEGLEQLEAQTNFT | (SEQ ID NO: 11) |
| KRELQVLYRGFKNECPSGVVNEDTFKQIYAQFFPHGDASTYAHYLFNAFDTTQTGSV | |
| KFEDFVTALSILLRGTVHEKLRWTFNLYDINKDGYINKEEMMDIVKAIYDMMGKYTY | |
| PVLKEDTPRQHVDVFFQKMDKNKDGIVTLDEFLESCQEDDNIMRSLQLFQNVM | |

KChIP1.2

(The amino acid sequence derived from splice variant 2 (KChIP1.2), the underlined amino acids are coded by exon Ins-r.)

| | |
|---|---|
| MGAVMGTFSSLQTKQRRPSKDIAWWYYQYQRDKIEDELEMTMVCHRPEGLEQLEA | (SEQ ID NO: 12) |
| QTNFTKRELQVLYRGFKNECPSGVVNEDTFKQIYAQFFPHGDASTYAHYLFNAFDTT | |
| QTGSVKFEDFVTALSILLRGTVHEKLRWTFNLYDINKDGYINKEEMMDIVKAIYDMM | |
| GKYTYPVLKEDTPRQHVDVFFQKMDKNKDGIVTLDEFLESCQEDDNIMRSLQLFQNV | |
| M | |

KChIP1.4

(The amino acid sequence derived from splice variant 4 (KChIP1.4).)

| | |
|---|---|
| MVCHRPEGLEQLEAQTNFTKRELQVLYRGFKNECPSGVVNEDTFKQIYAQFFPHGDA | (SEQ ID NO: 13) |
| STYAHYLFNAFDTTQTGSVKFEDFVTALSILLRGTVHEKLRWTFNLYDINKDGYINKE | |
| EMMDIVKAIYDMMGKYTYPVLKEDTPRQHVDVFFQKMDKNKDGIVTLDEFLESCQE | |
| DDNIMRSLQLFQNVM | |

Identification of SNPs and Microsatellites

In order to identify SNPs across KChIP1, all exons of KChIP1 and their flanking regions were sequenced on 94 non-obese diabetic patients. As a consequence, 31 SNPs were identified (Table 9). Additional SNPs were identified across the gene by selecting SNPs from the public domain (US National Center for Biotechnology Information's SNP database) and designing SNP assays for them. (Table 10).

SNPs on 470 non-obese diabetics and 658 population-based controls using a method for detecting SNPs with fluorescent polarization template-directed dye-terminator incorporation (SNP-FP-TDI assay) were genotyped (Chen, X., Zehnbauer, B., Gnirke, A. & Kwok, P. Y. Proc. Natl. Acad. Sci. USA 94, 10756-10761 (1997)).

Association Study of Genes in Region B

All the genes in and around Region B (LCP2, KCNMB1, KChIP1, GABRP and RANBP17) individually for association to non-obese diabetes were tested. In the analysis of each gene, all SNPs identified, and previously typed microsatellites, in and close to that gene were included. The association analysis was carried out in the same way as the locus-wide association, i.e., using the iterative approach, searching for haplotypes, shorter than 300 kb, that showed strongest association to the disease was then completed.

The strongest association observed was for KChIP1. For KChIP1, 25 markers, 7 microsatellites and 18 SNPs, for association (Table 11) were tested. The strongest association signal was observed in the 3′-end of the gene; a three marker haplotype with a P-value=9.2×10⁻⁵, relative risk 12, and allelic frequency 3.6% and 0.3% in the patient and control cohorts, respectively. This haplotype, which extends over the last 8 exons of KChIP1, from 169.96 to 170.11 Mb, is listed in Table 4 as D1. We also observed another haplotype in the same region that showed association to non-obese diabetes, albeit less significant than D1, with a P-value=0.037, relative risk 1.69 and allelic frequency 7.8% and 4.8% in the patient and the control cohorts, respectively. This haplotype is labelled D2 in Table 4. For risk haplotypes, the corresponding population attributable risk is PAR=4.9% for D1 and PAR=4.7% for D2. However, as D1 and D2 are independent haplotypes, i.e., they do not appear jointly on the same chromosome, their population attributable risk can be added together.

TABLE 4
Table 4: Microsatellite and SNP haplotype association within KChIP1.
The two independent haplotypes D1 and D2 are located in the 3′-end
of the gene, from 169.96-170.11 Mb. Shown are results of a test of
association for non-obese diabetics vs population controls for both haplotypes
in a cohort of Icelandic diabetics (top) and a replication in a cohort
of Danish diabetics (bottom). Note that we report one-sided P-values for
the test on the Danish cohort as that is a replication of association
results previously observed in the Icelandic cohort.

| | P-Value | RR | Aff. frq. | Ctrl. frq | Haplotype |
|---|---|---|---|---|---|
| Icelandic | | | | | |
| D1 | 9.20E−05 | 12 | 0.036 | 0.003 | −4 DG5S13 C SG05S176 0 |
| | | | | | D5S625 |
| D2 | 0.037 | 1.69 | 0.078 | 0.048 | 0 DG5S124 C KCP_1152 C |
| | | | | | KCP_2649 T KCP_4976 A |
| | | | | | KCP_16152 |
| Danish | | | | | |
| D1 | 0.06 | 0.0008 | 0.001% | 0.06 | −4 DG5S13 C SG05S176 0 |
| | | | | | D5S625 |
| D2 | 0.0004 | 2.59 | 10.5% | 0.0004 | 0 DG5S124 C SG05S176 C |
| | | | | | SG05923 T SG05S187 A |
| | | | | | SG05S948 |

Replication in a Cohort of Danish Diabetics

The markers that define the two at-risk haplotypes, D1 and D2, in a cohort of 149 non-obese Danish females that have been diagnosed with diabetes and/or measured >7 mM glucose who participated in a Danish PERF (Prospective Epidemiological Risk Factors) study were typed. As controls, 346 females from the same study that answered no to a question about their diabetes status and/or measured <7 mM glucose were used.

The results of the association test for the two at-risk haplotypes, identified in the Icelandic diabetes cohort, are listed in Table 4. Both haplotypes appear in higher frequency in the non-obese Danish diabetics than in the control cohort. For haplotype D1, the association to non-obese diabetes is only marginally significant, with a one-sided P-value=0.05, and the relative risk of the at-risk haplotype is RR=3.0, somewhat less than is observed for the Icelandic non-obese diabetics. Note, however, that the estimated frequency of haplotype D1 is very low, especially in the control cohorts, hence the estimates of the relative risk are not very reliable. For haplotype D2, on the other hand, we do observe a statistically significant association with a one-sided P-value=0.002 and relative risk=2.74. Note that as the test of association of haplotypes D1 and D2 are attempts to replicate the association we have observed for Icelandic non-obese diabetics, it is appropriate to report one-sided P-values for those tests.

Additional SNP Genotyping for KChIP1

Having observed association to the 3′-end of KChIP1, both in Icelandic and Danish non-obese diabetics, 94 Icelandic individuals, ⅓ non-obese type II diabetes patients with the observed haplotype D1, ⅓ additional non-obese type II diabetes patients and ⅓ controls, were subsequently sequenced. The purpose of the sequencing was to identify additional SNPs. 725 SNPs (Table 12) were identified. Many of those SNPs were completely correlated so several redundant SNPs from further genotyping were removed. Some SNPs with very low minor allele frequencies were also ignored. Of the 725 identified SNPs plus what was originally identified, 108 were selected for further genotyping in the Icelandic cohort (Table 13).

A single-marker test of association was performed on non-obese diabetes for each of the additional SNPs typed, although none of the SNPs showed a strong association. However, it was observed that three of the SNPs, KCP_{—}197678, KCP_{—}197775 and KCP_{—}202795, increased the specificity of haplotype D2, if added to that haplotype, while still retaining most of its sensitivity. This is shown in Table 5, both for the association in the Icelandic and in the Danish cohorts. This increases the value of the at-risk haplotype as a diagnostic tool. Note that the three SNPs are very correlated to each other, with pairwise correlation coefficients D′≈0.96 and R²≈0.9, hence the association of haplotypes D3, D4 and D5 to non-obese diabetes should be considered as a single observation.

In addition to the refinement of the at-risk haplotype D2, another refinement of the at-risk haplotype was observed, consisting of three SNPs only, that was very correlated with the three at-risk haplotoypes, D3, D4 and D5, with pairwise correlation coefficients D′≈0.83 and R²≈0.59. This haplotype is included in Table 5 as D6.

TABLE 5
Table 5: Microsatellite and SNP haplotype association within KChIP1. Shown is association of
the at-risk haplotype D2, and of further refinements of that haplotype; haplotypes D3, D4 and
D5, to non-obese diabetes. This is shown both for the Icelandic and the Danish cohorts and, as
in Table 4, we report one-sided P-values for the association test in the Danish cohort. Finally,
we include the result of association to non-obese diabetes, in the Icelandic cohort, of a 3 SNP
haplotype, D6, that is strongly correlated with the at-risk haplotoypes D3, D4 and D5.

| | P-Value | RR | PAR | Aff. frq. | Ctrl. frq | Haplotype |
|---|---|---|---|---|---|---|
| Icelandic | | | | | | |
| D2 | 0.037 | 1.69 | 6.3% | 0.078 | 0.048 | 0 DG5S124 C KCP_1152 C KCP_2649 T |
| | | | | | | KCP_4976 A KCP_16152 |
| D3 | 0.022 | 2.19 | 5.5% | 0.052 | 0.024 | 0 DG5S124 C KCP_1152 C KCP_2649 T |
| | | | | | | KCP_4976 A KCP_16152 T KCP_197678 |
| D4 | 0.052 | 2.03 | 4.6% | 0.046 | 0.023 | 0 DG5S124 C KCP_1152 C KCP_2649 T |
| | | | | | | KCP_4976 A KCP_16152 T KCP_197775 |
| D5 | 0.023 | 2.14 | 5.5% | 0.052 | 0.025 | 0 DG5S124 C KCP_1152 C KCP_2649 T |
| | | | | | | KCP_4976 A KCP_16152 C KCP_202795 |
| D6 | 0.054 | 1.77 | 4.0% | 0.046 | 0.027 | A KCP_173982 C KCP_15400 C |
| | | | | | | KCP_18069 |
| Danish | | | | | | |
| D2 | 0.002* | 2.74 | 12.0% | 0.098 | 0.038 | 0 DG5S124 C KCP_1152 C KCP_2649 T |
| | | | | | | KCP_4976 A KCP_16152 |
| D3 | 0.0046* | 2.60 | 9.0% | 0.076 | 0.030 | 0 DG5S124 C KCP_1152 C KCP_2649 T |
| | | | | | | KCP_4976 A KCP_16152 T KCP_197678 |
| D4 | 0.0004* | 3.69 | 11.3% | 0.078 | 0.023 | 0 DG5S124 C KCP_1152 C KCP_2649 T |
| | | | | | | KCP_4976 A KCP_16152 T KCP_197775 |
| D5 | 0.0002* | 3.67 | 11.7% | 0.084 | 0.024 | 0 DG5S124 C KCP_1152 C KCP_2649 T |
| | | | | | | KCP_4976 A KCP_16152 C KCP_202795 |

*One-sided P-value

Allele Numbering System

SNP alleles are indicated by the letters found in the DNA sequence. In general the alleles can be references by A=0, C=1, G=2 and T=3. For microsatellite alleles, the CEPH sample (Centre d'Etudes du Polymorphisme Humain, genomics repository) is used as a reference, the lower allele of each microsatellite in this sample is set at 0 and all other alleles in other samples are numbered according in relation to this reference. Thus allele 1 is 1 bp longer than the lower allele in the CEPH sample, allele 2 is 2 bp longer than the lower allele in the CEPH sample, allele 3 is 3 bp longer than the lower allele in the CEPH sample, allele 4 is 4 bp longer than the lower allele in the CEPH sample, allele −1 is 1 bp shorter than the lower allele in the CEPH sample, allele −2 is 2 bp shorter than the lower allele in the CEPH sample, and so on.

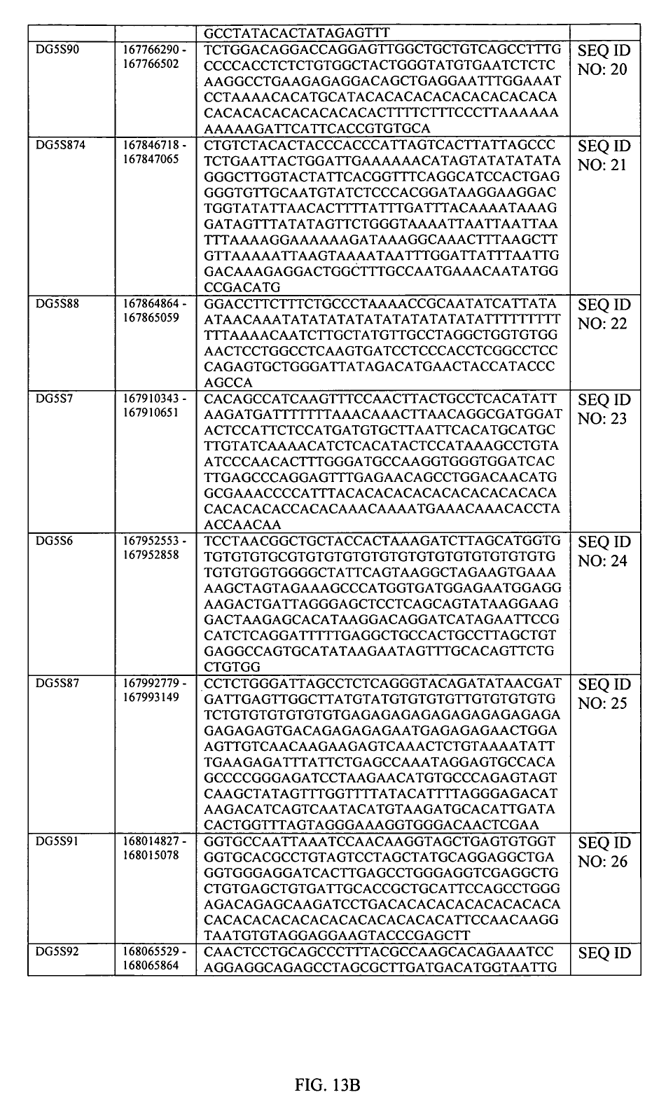

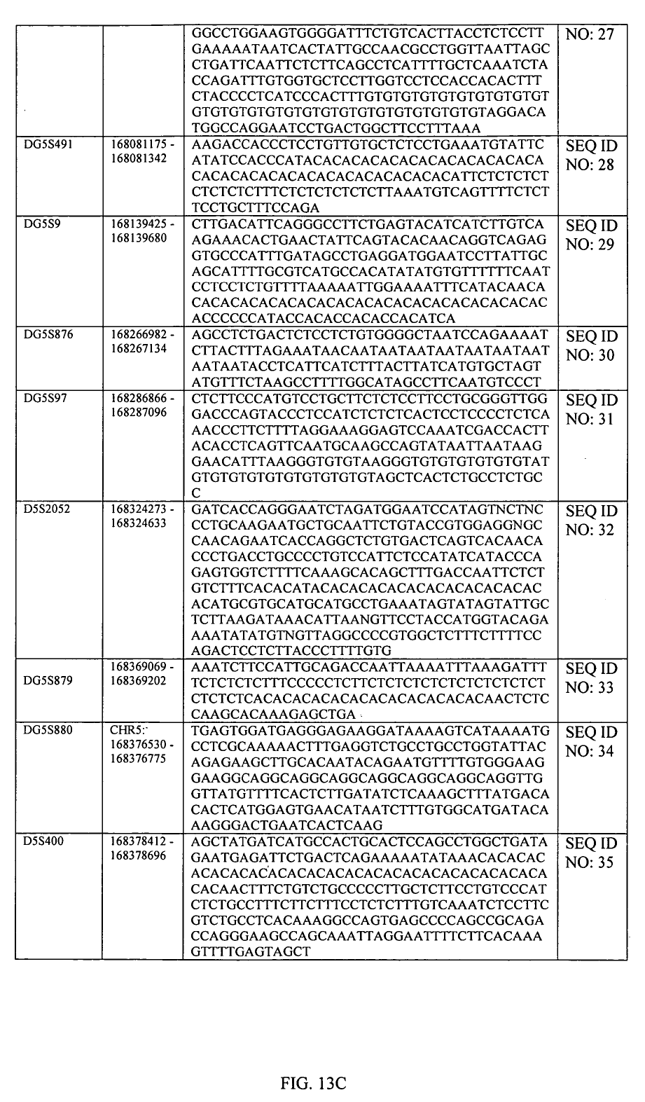

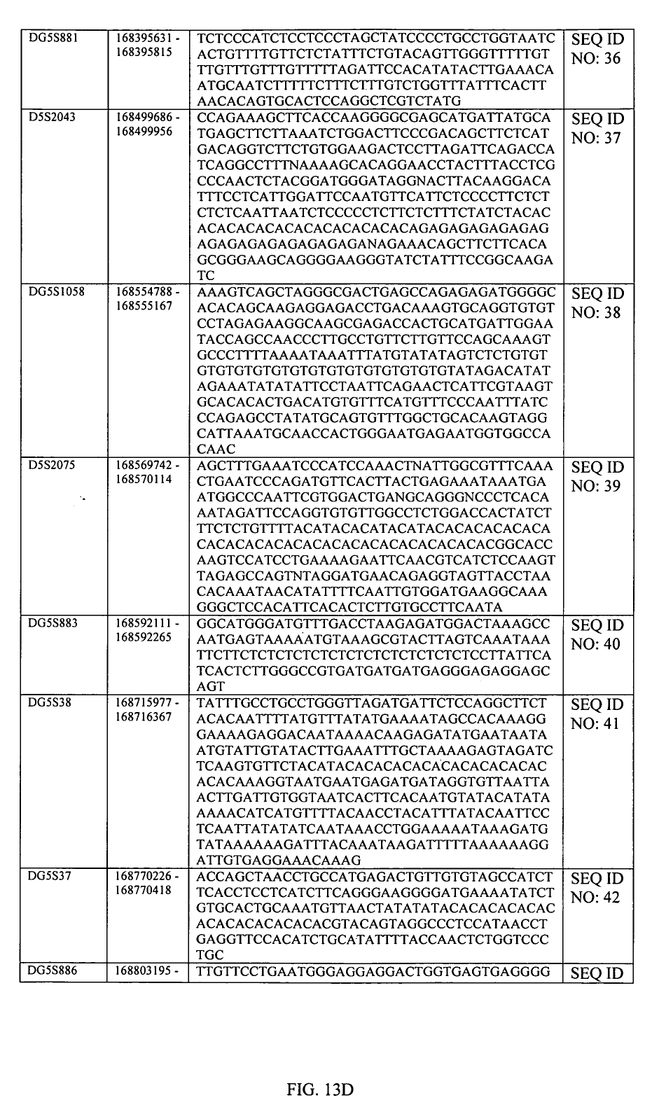

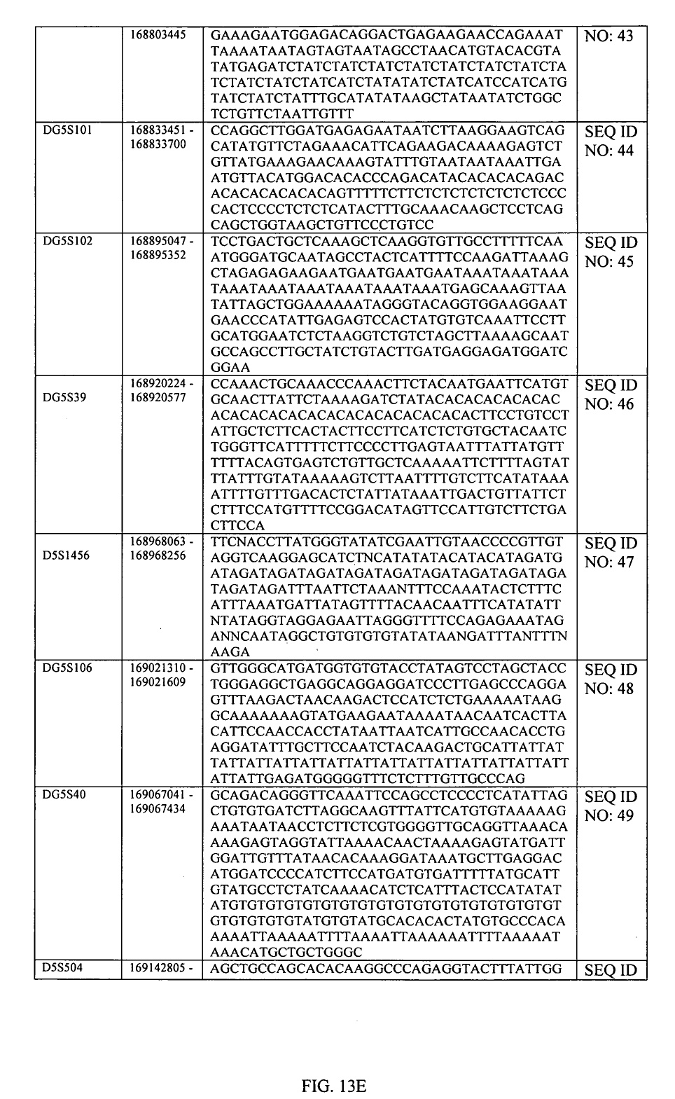

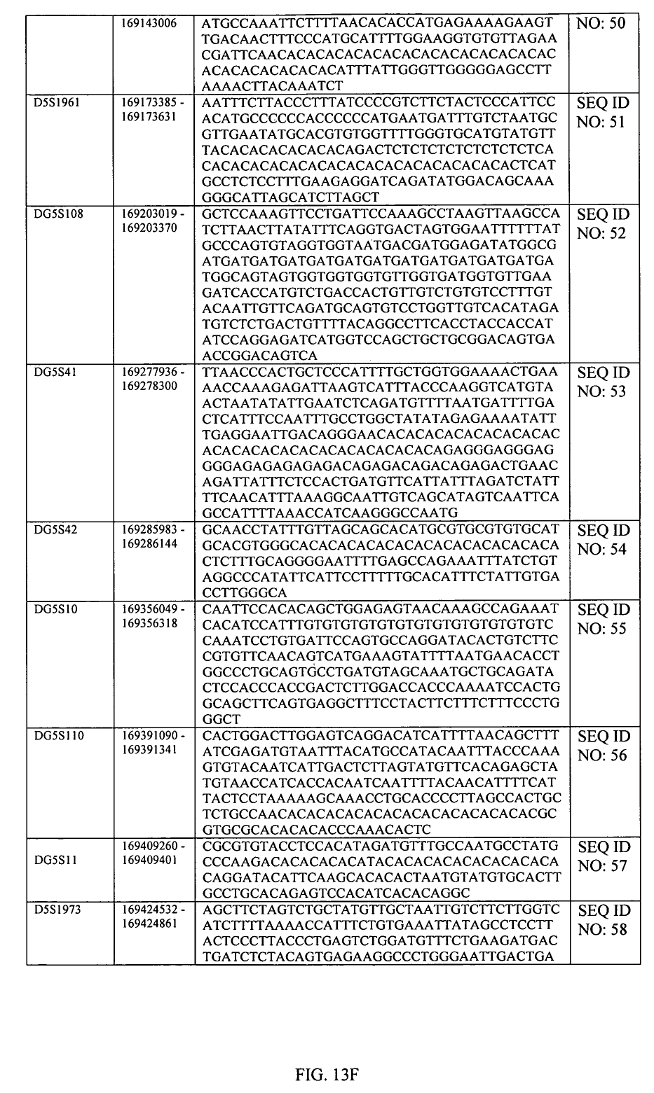

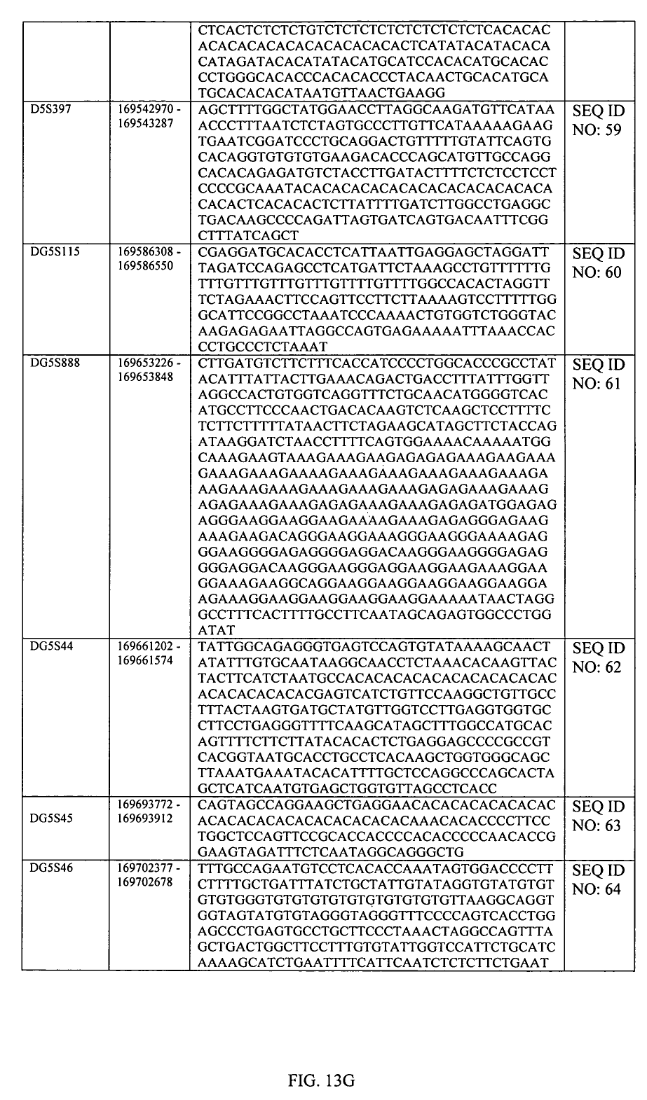

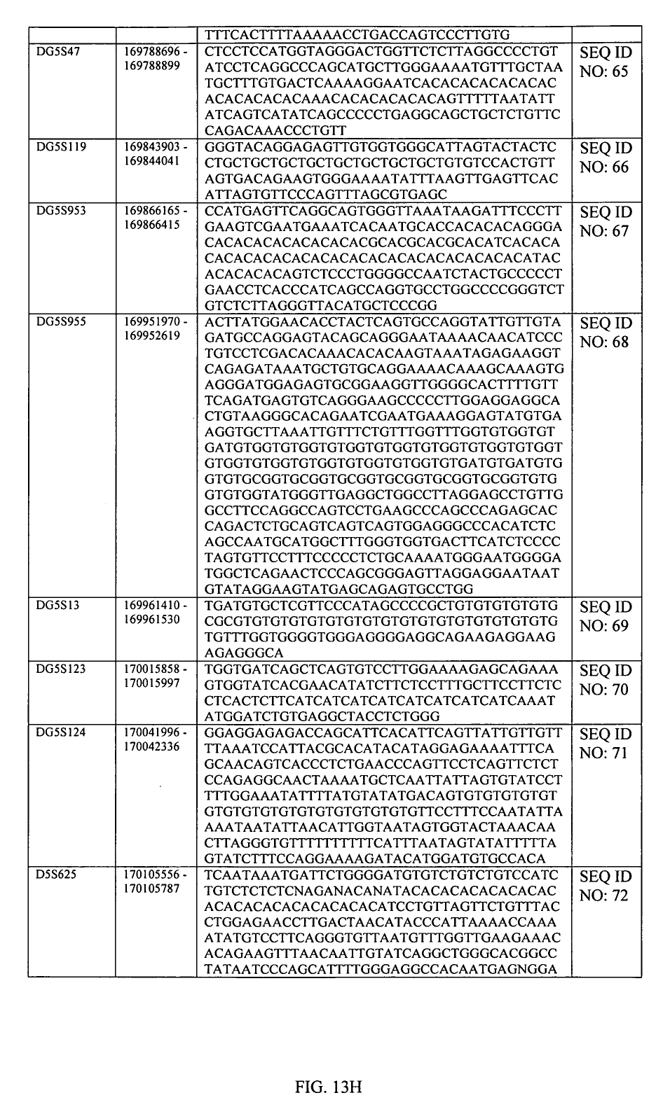

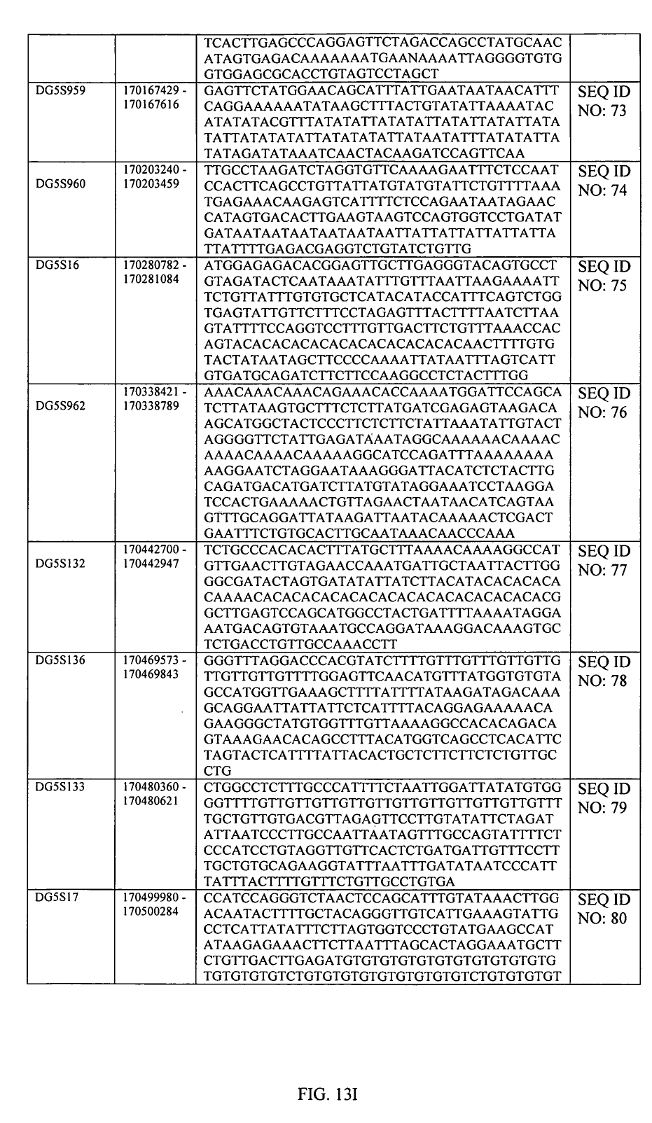

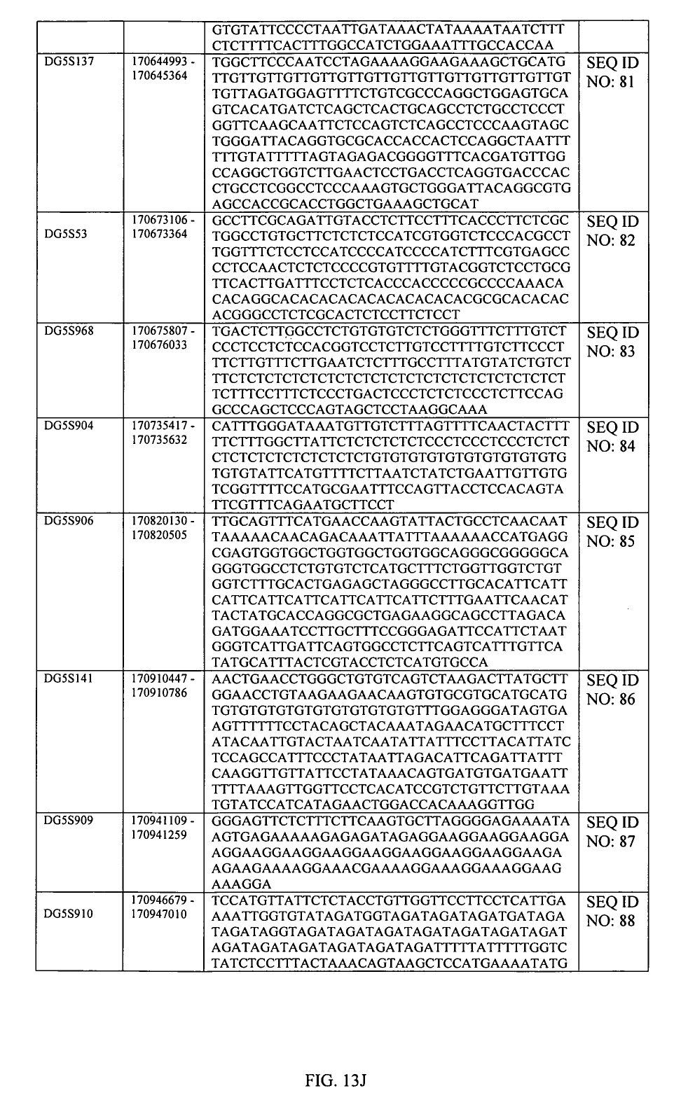

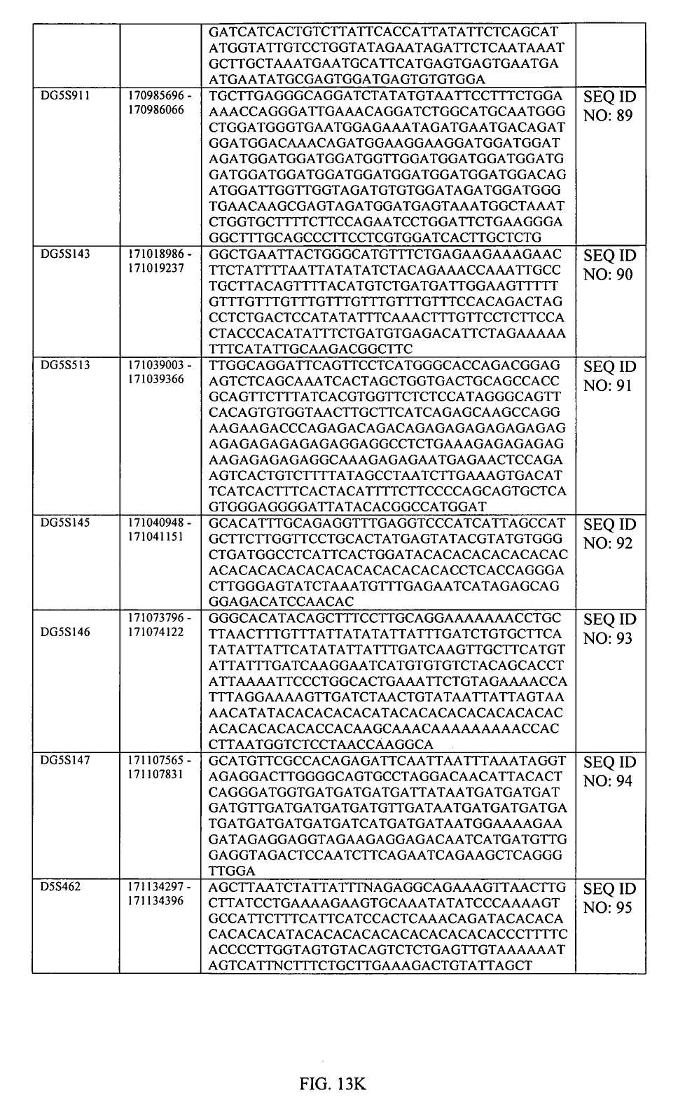

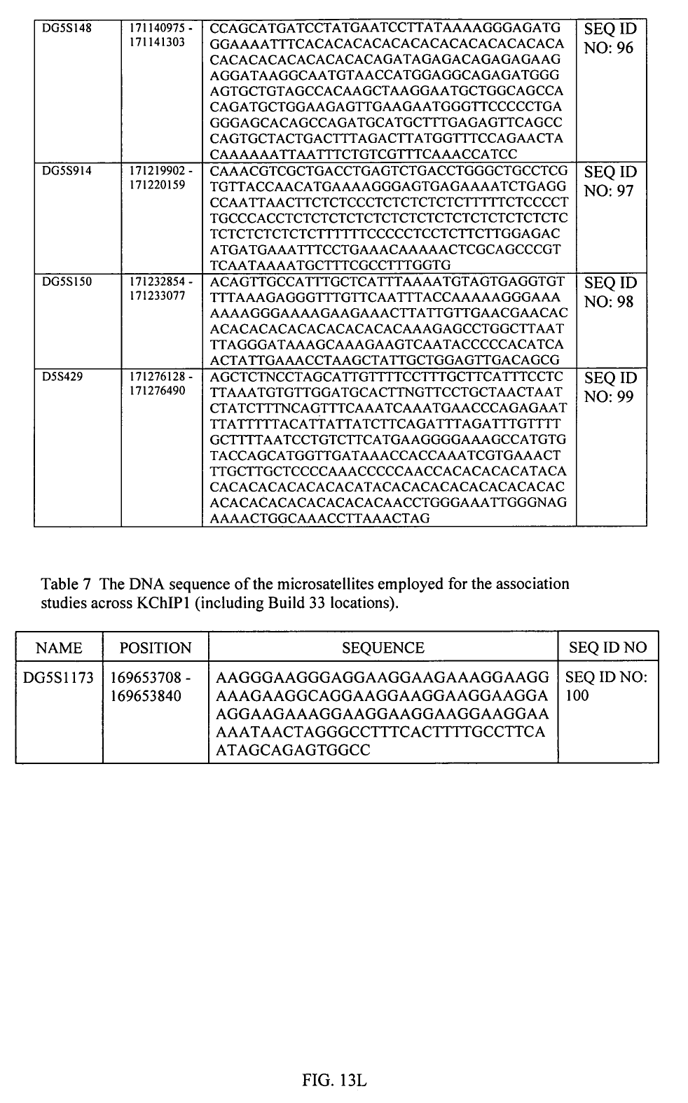

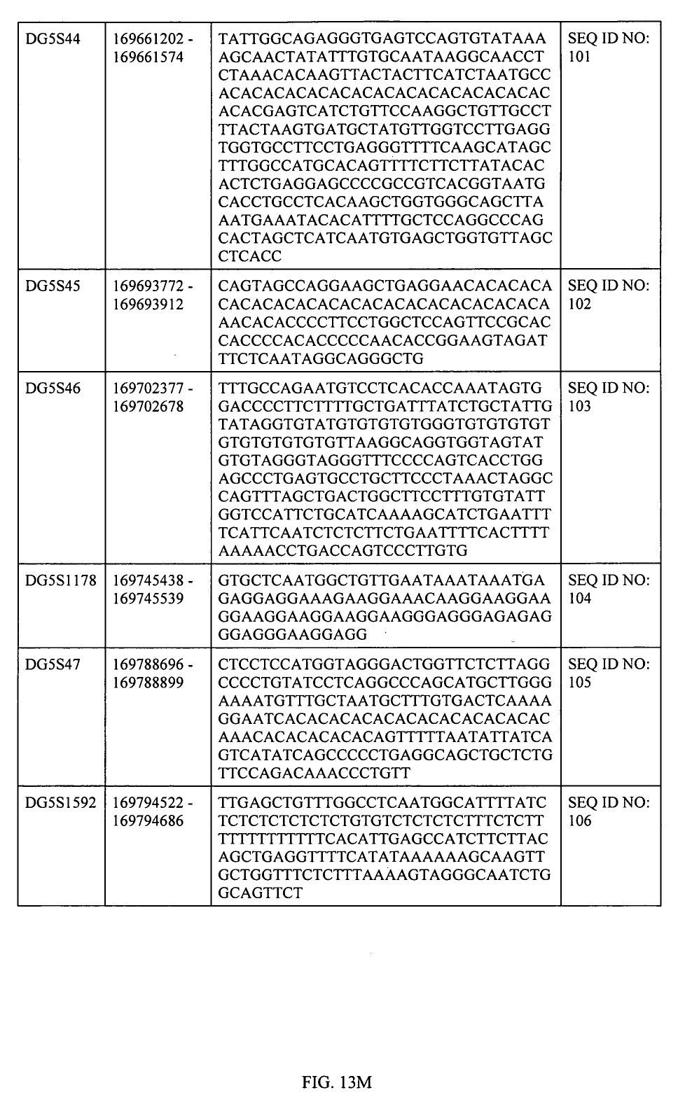

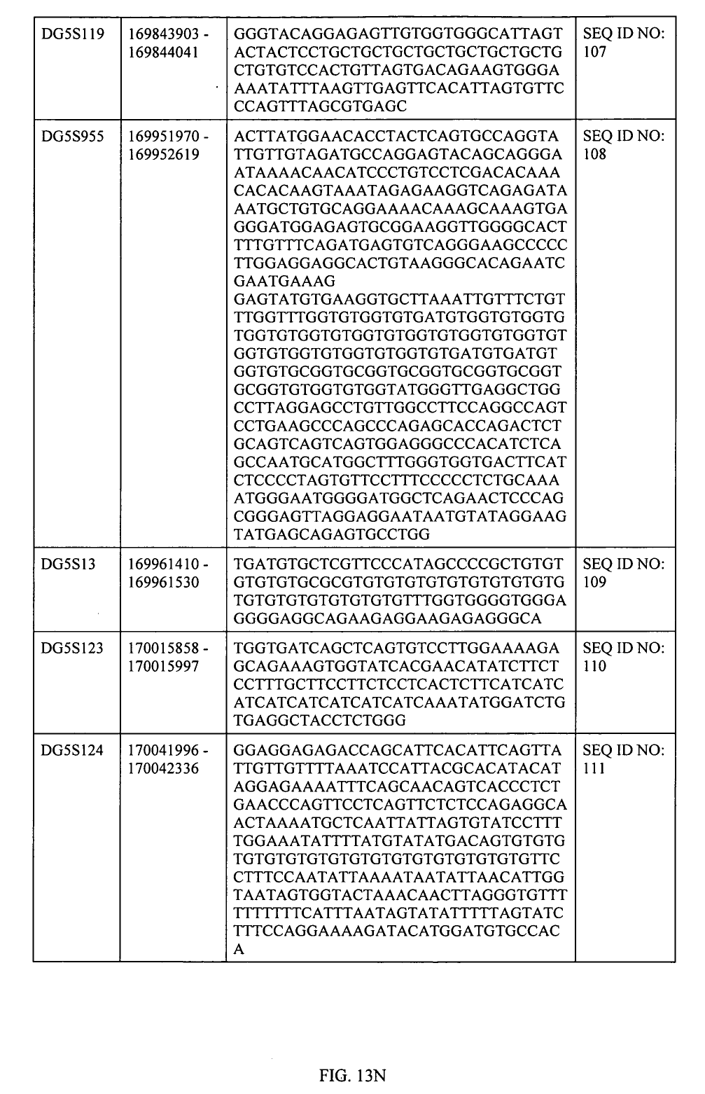

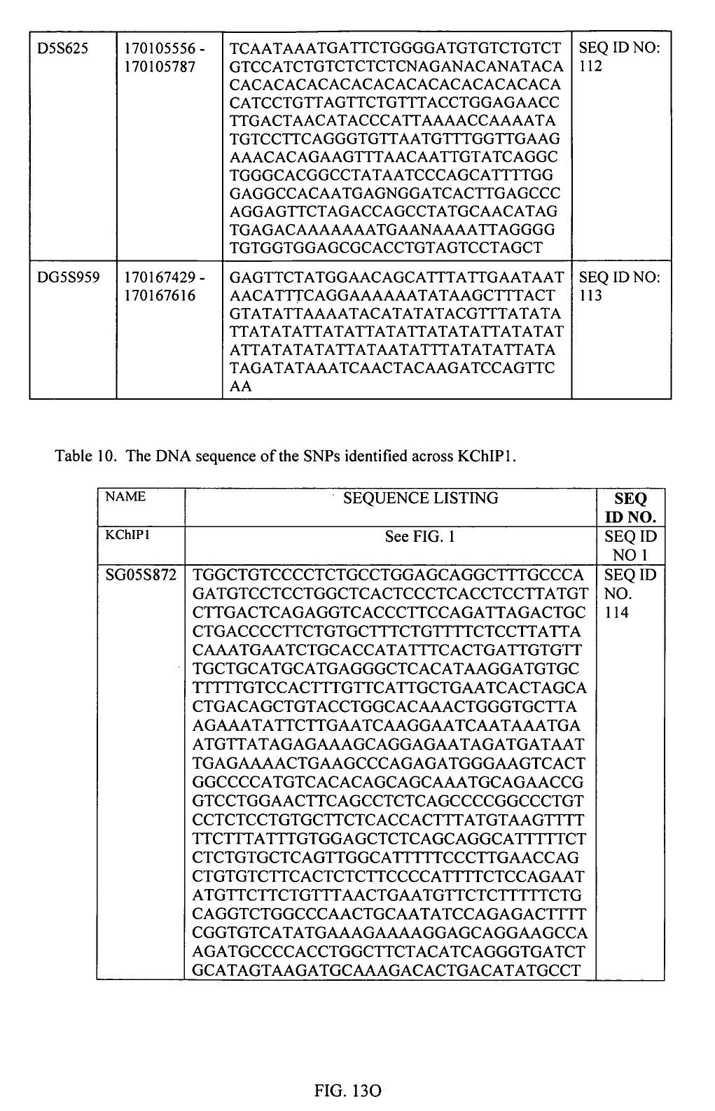

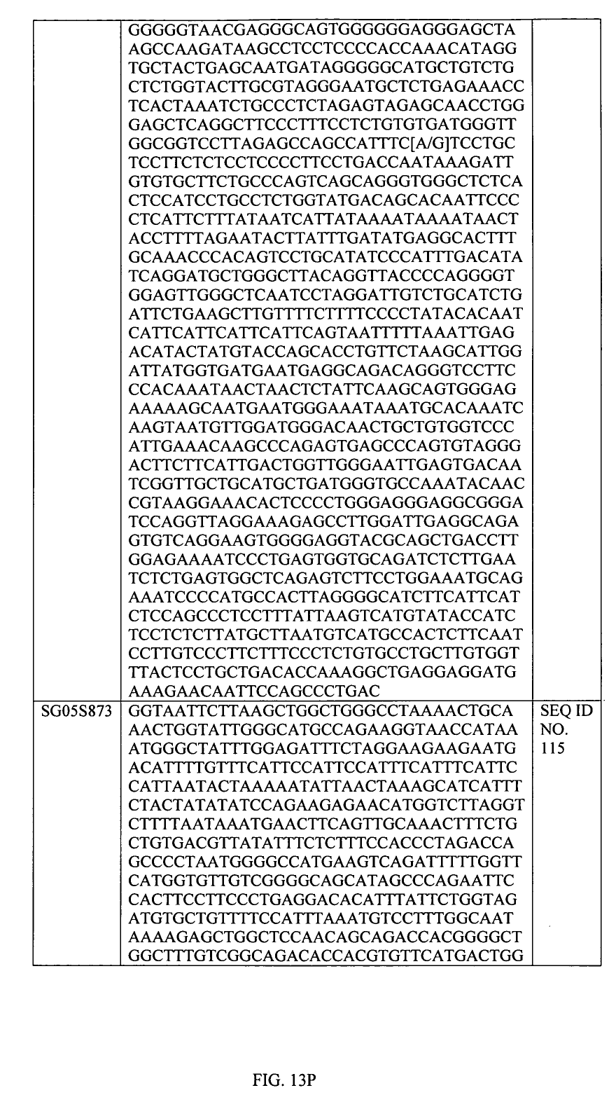

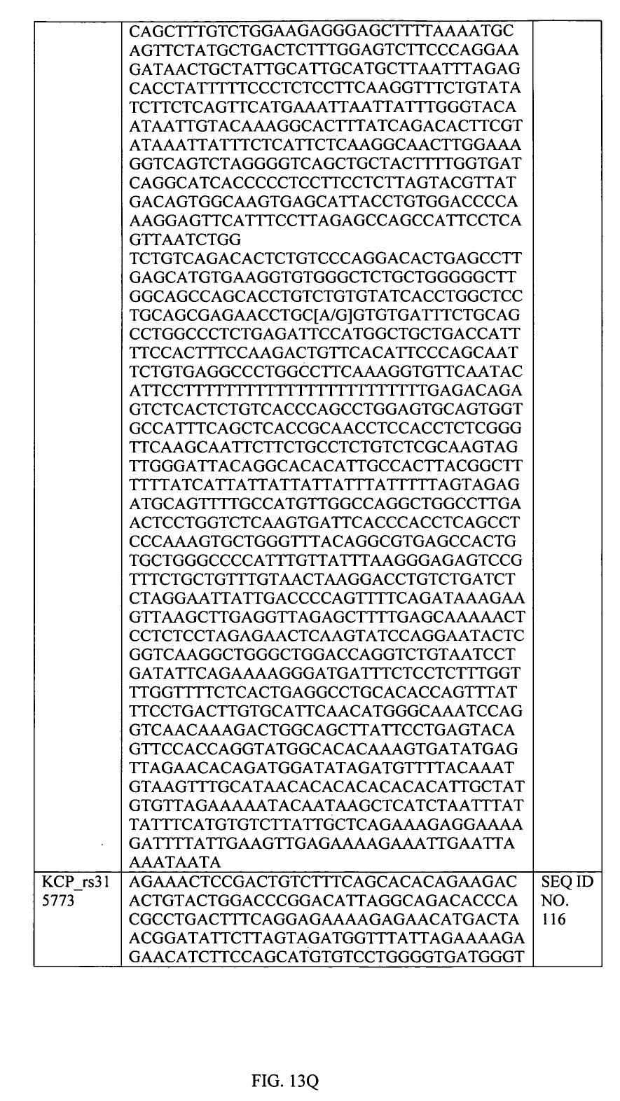

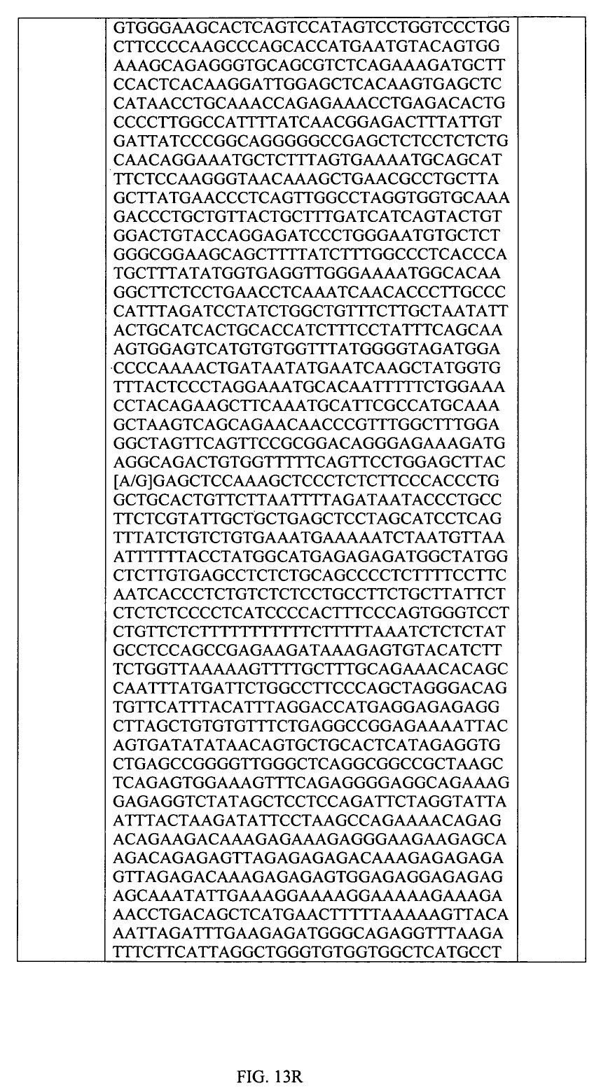

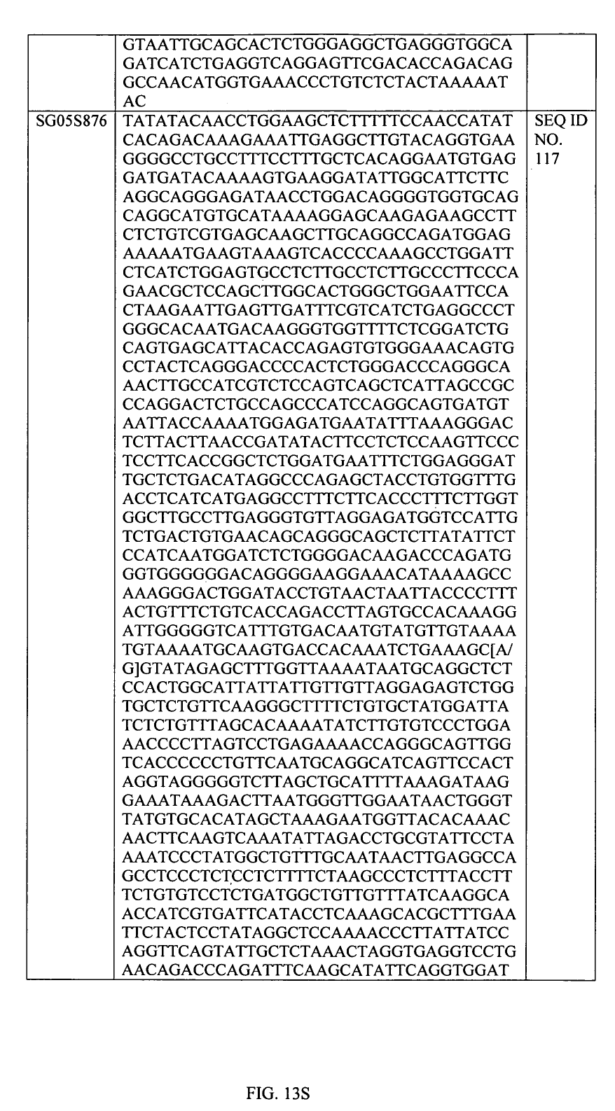

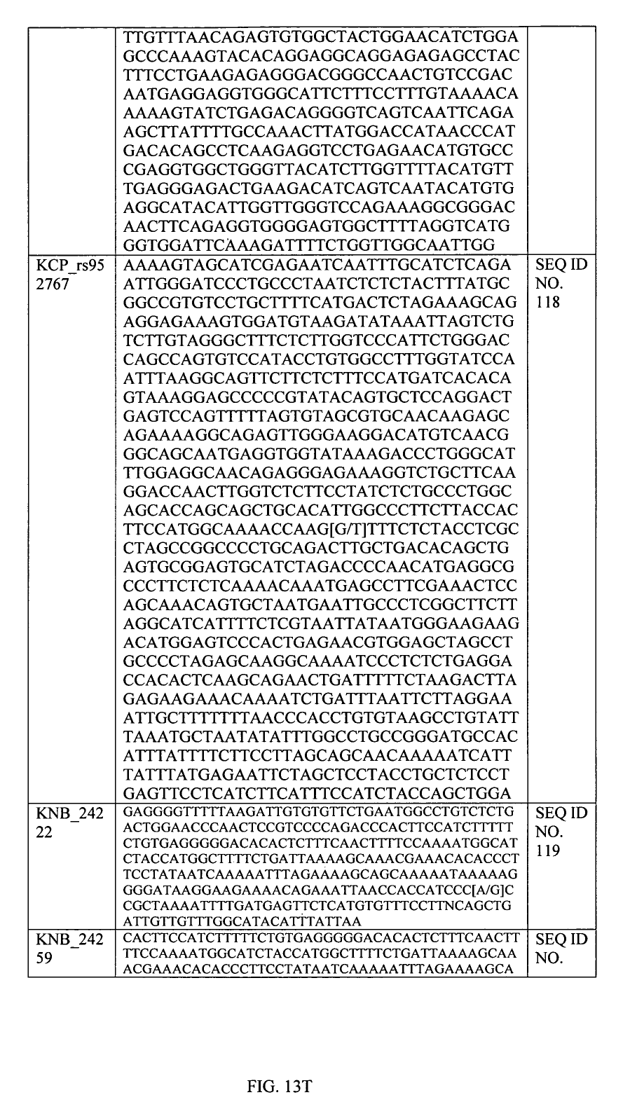

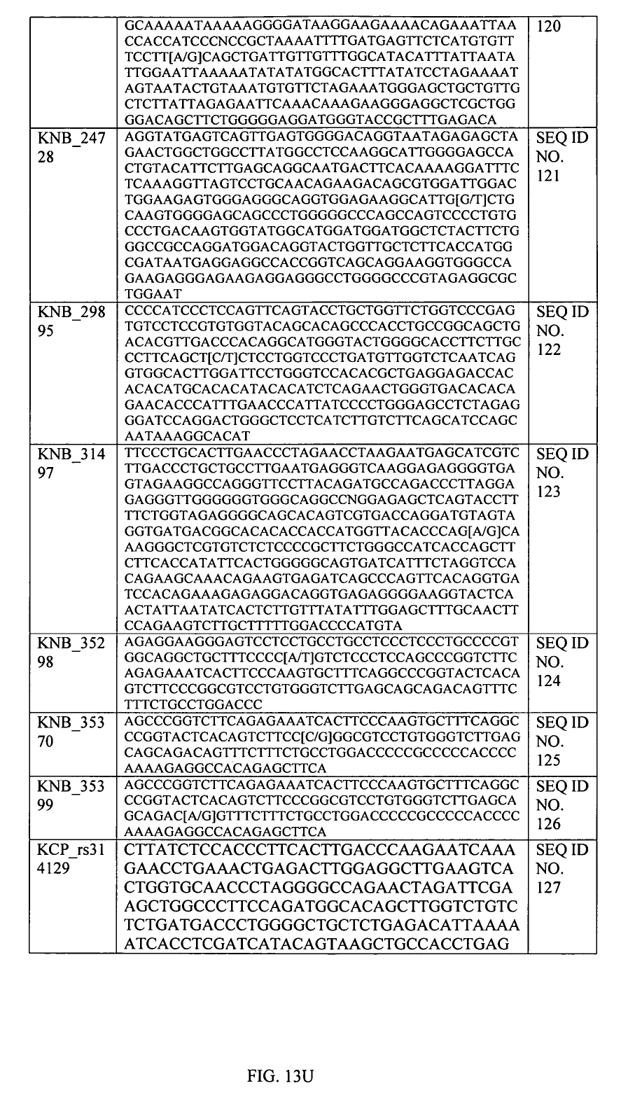

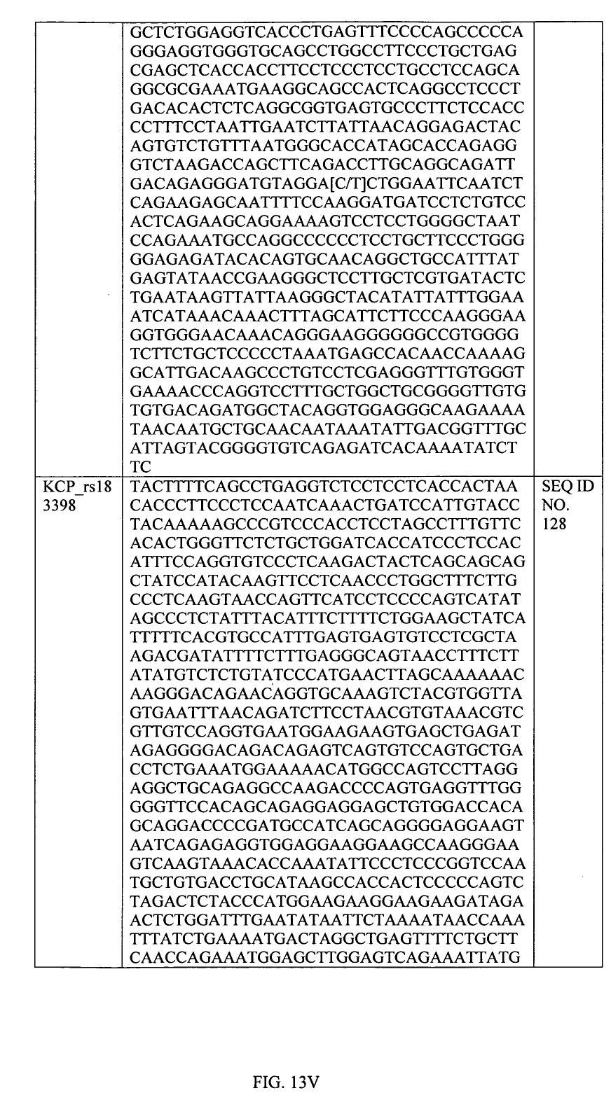

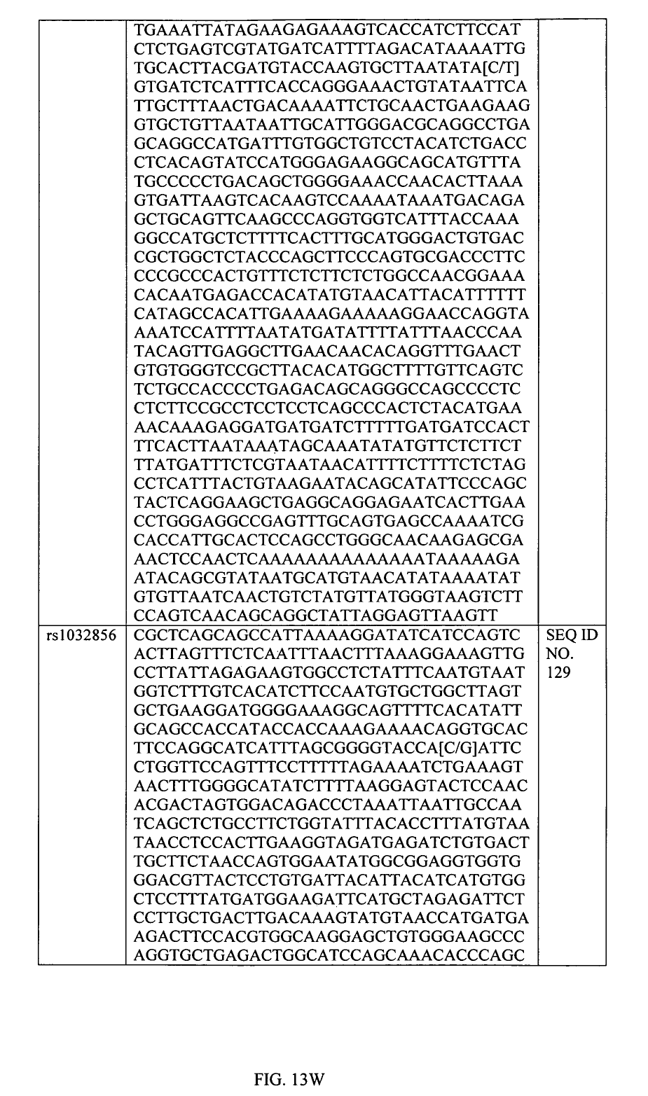

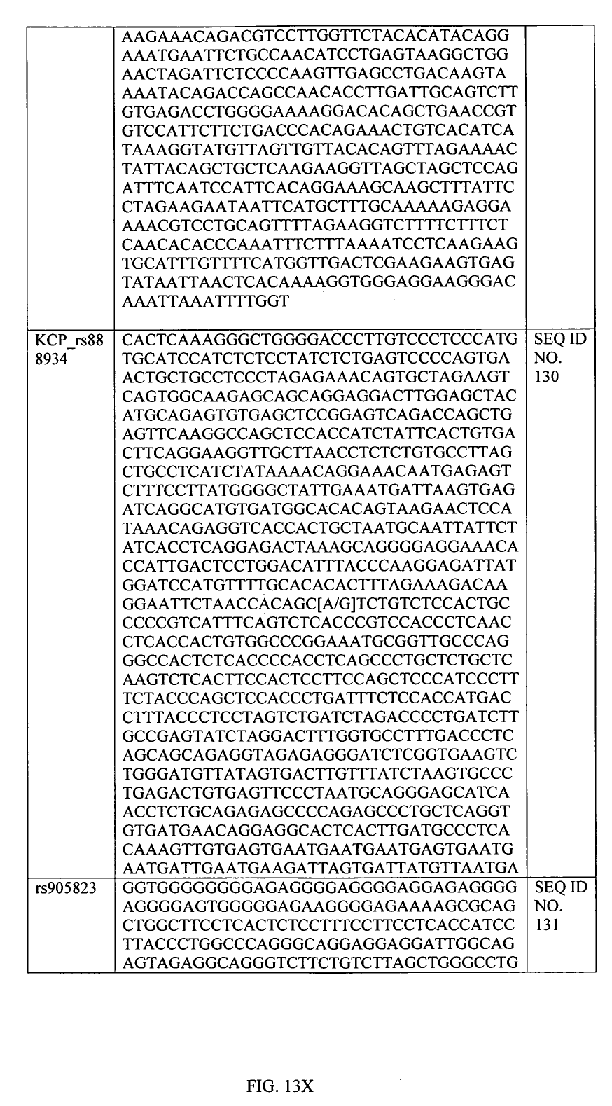

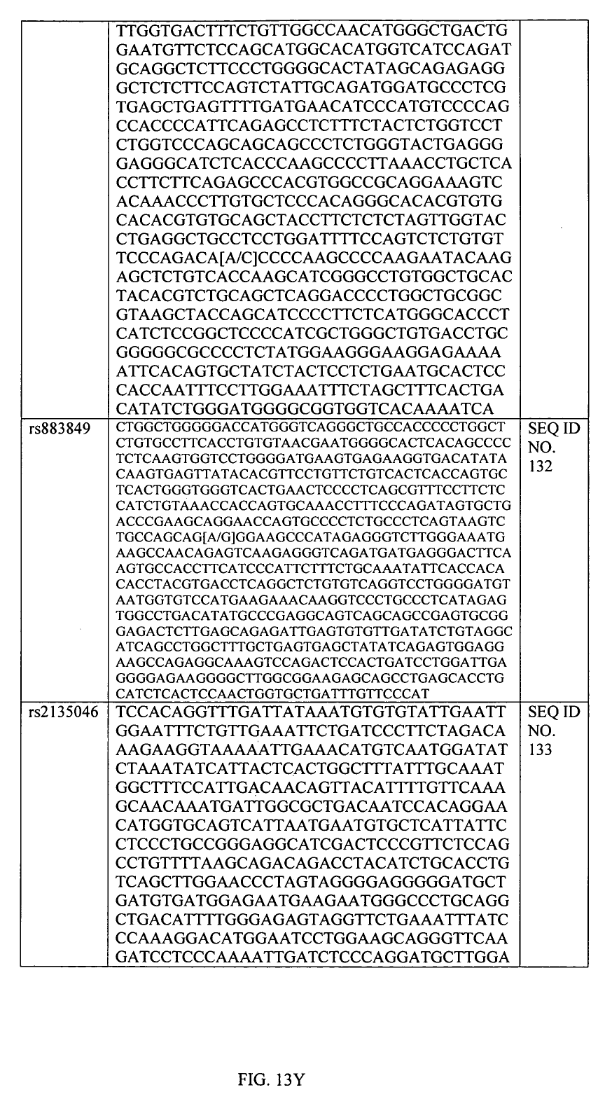

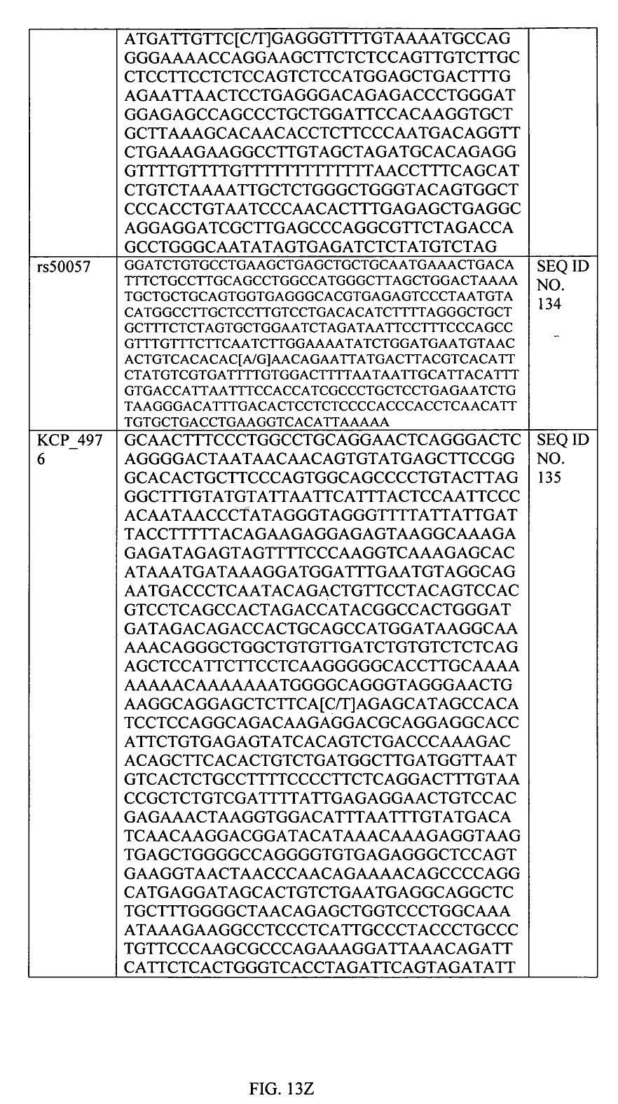

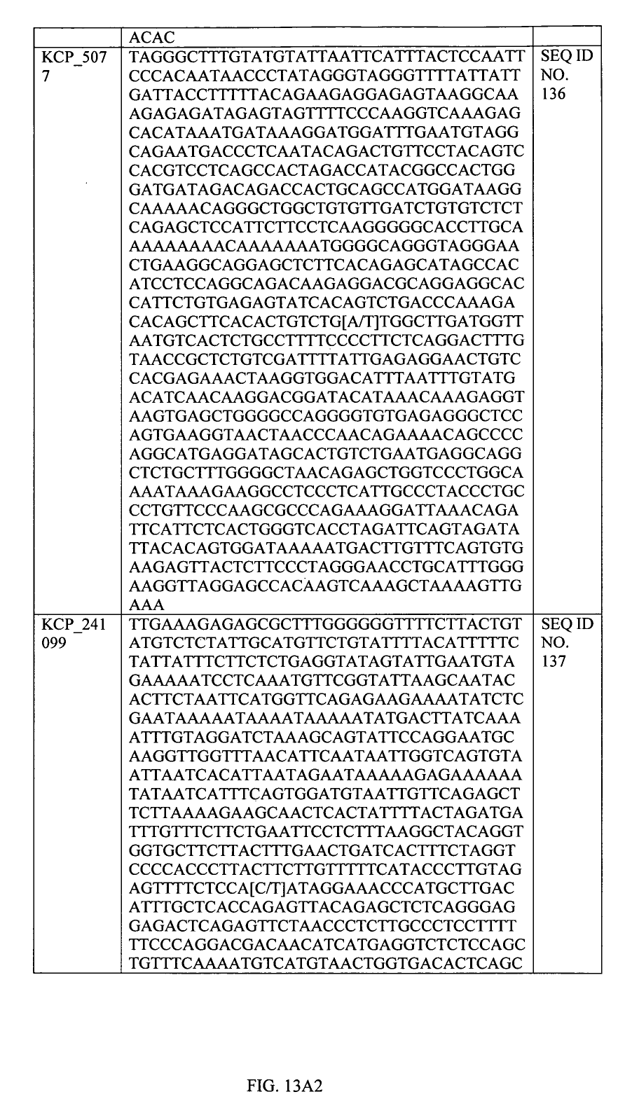

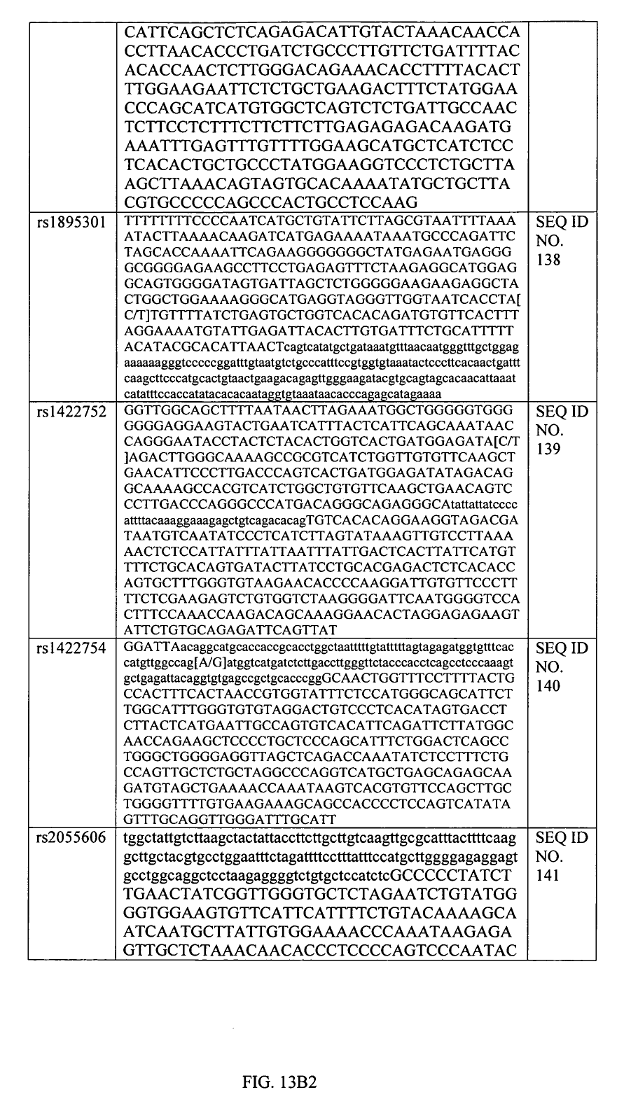

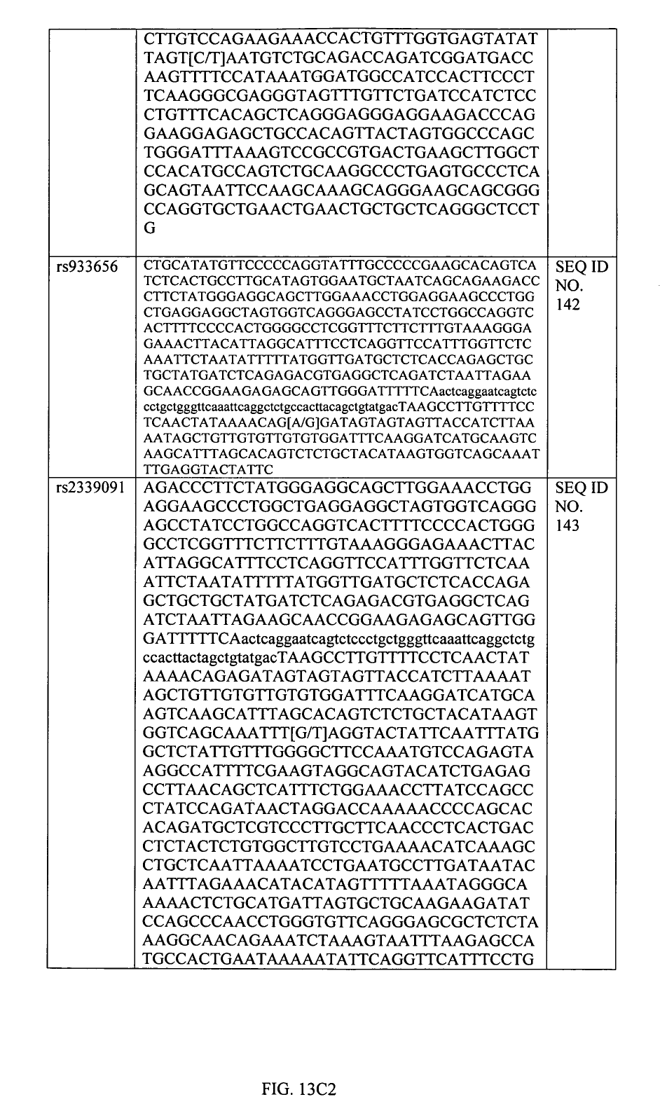

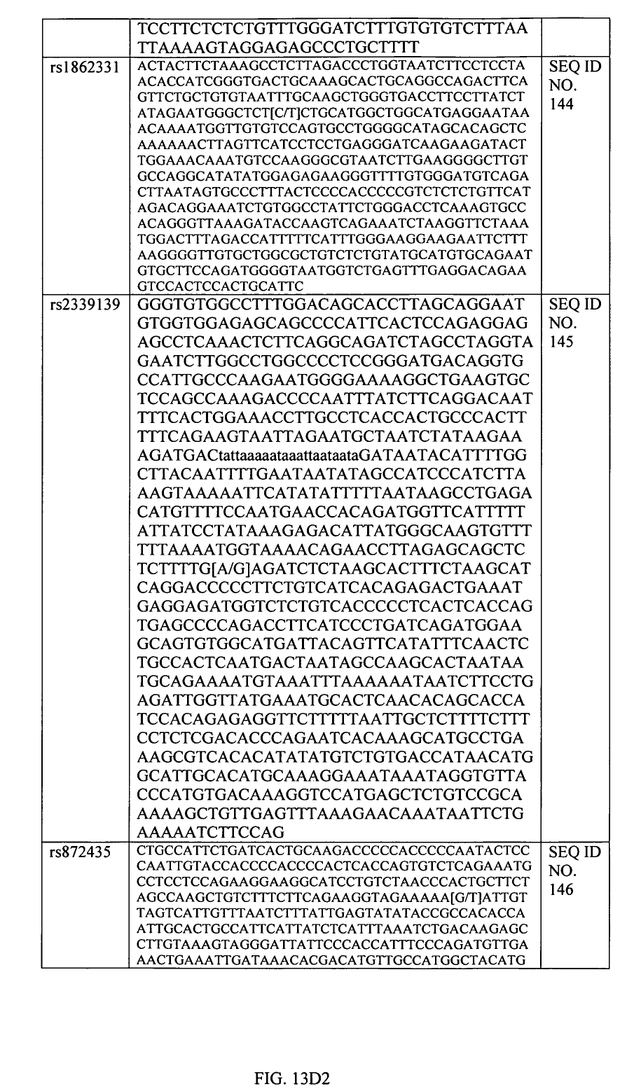

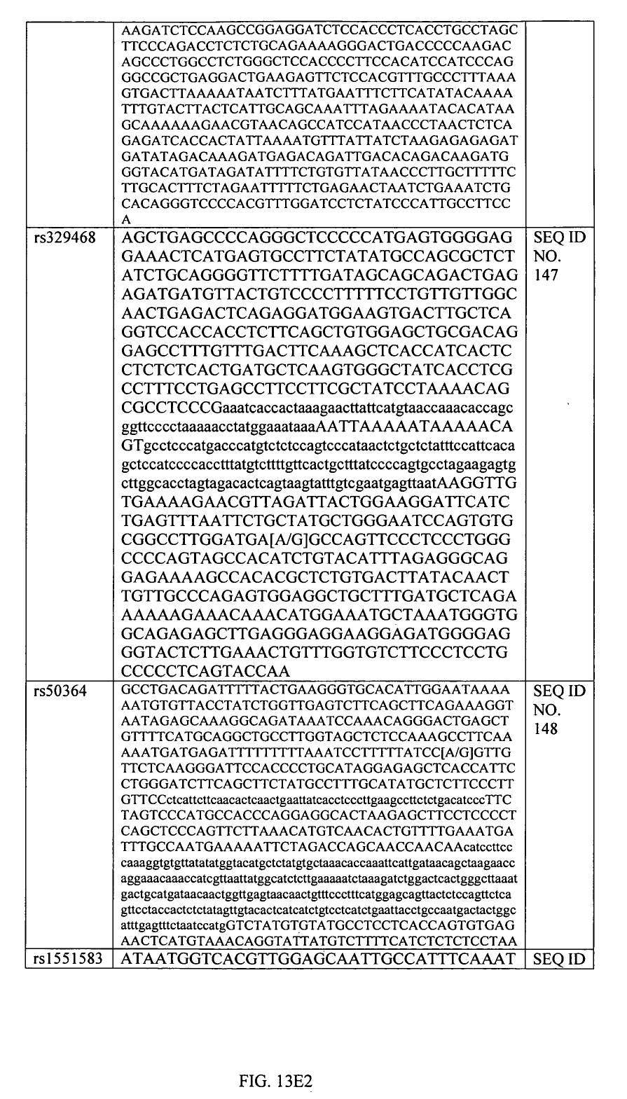

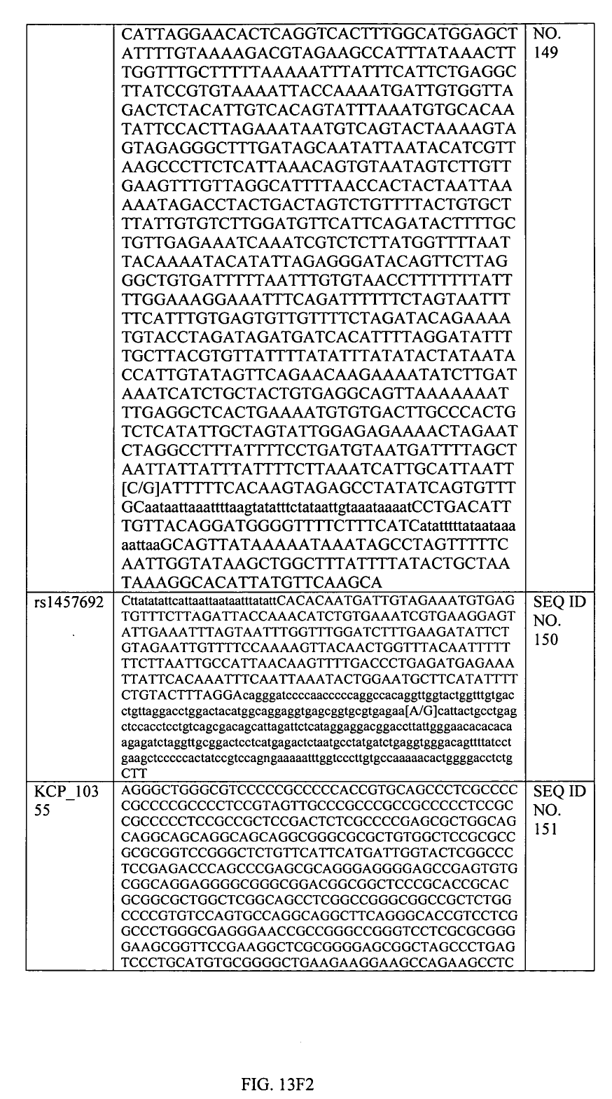

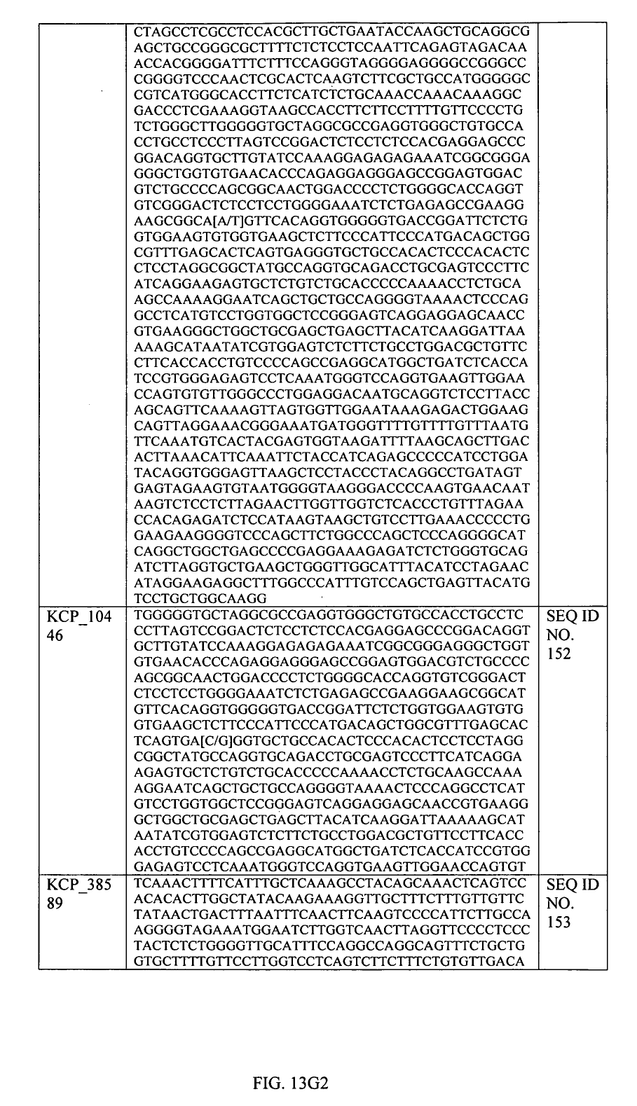

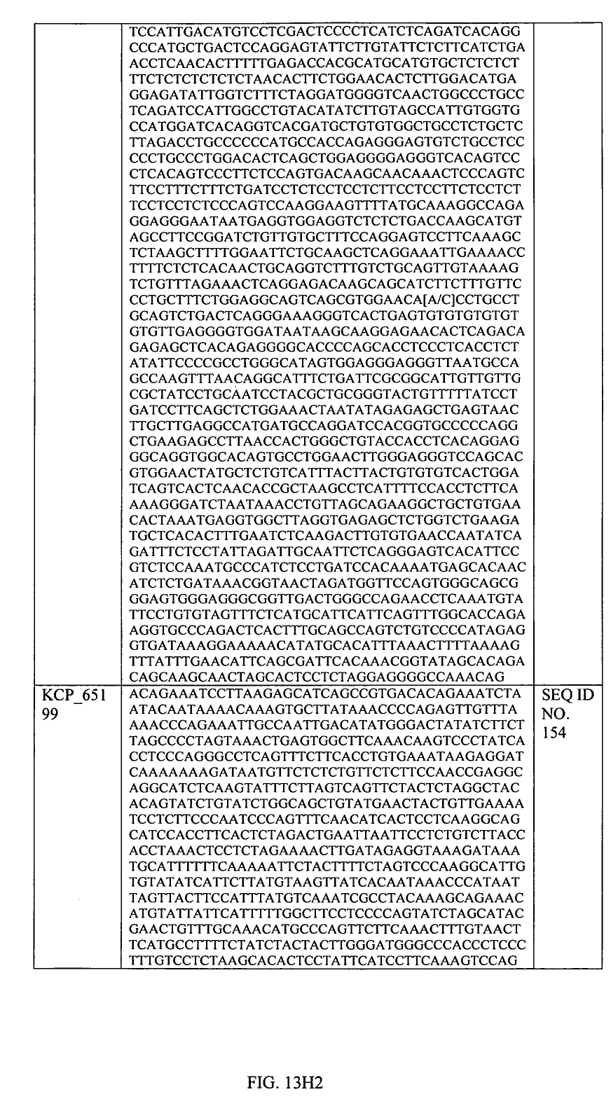

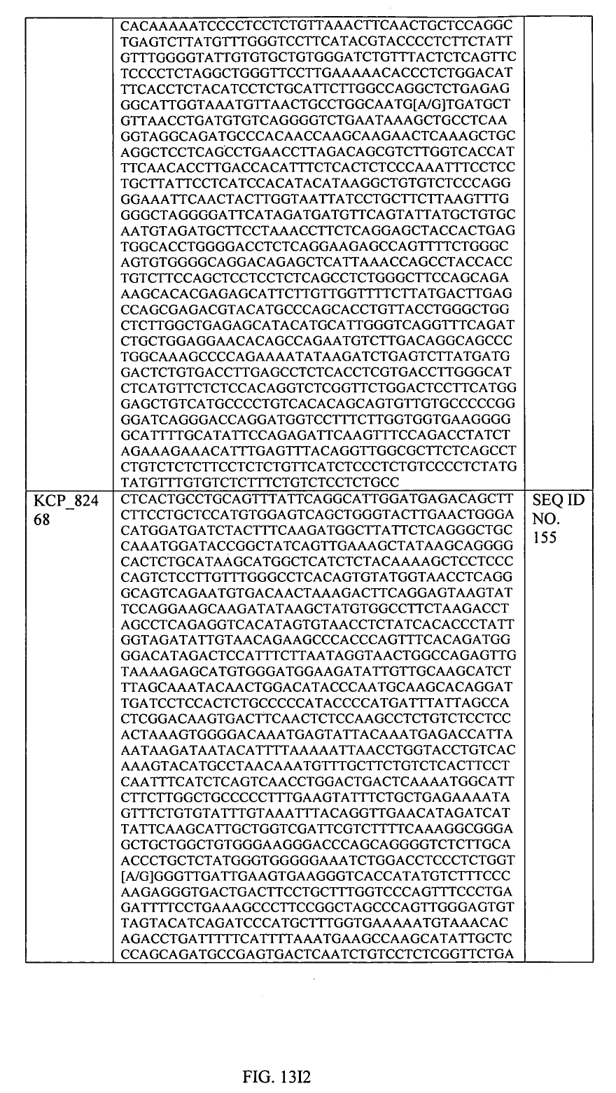

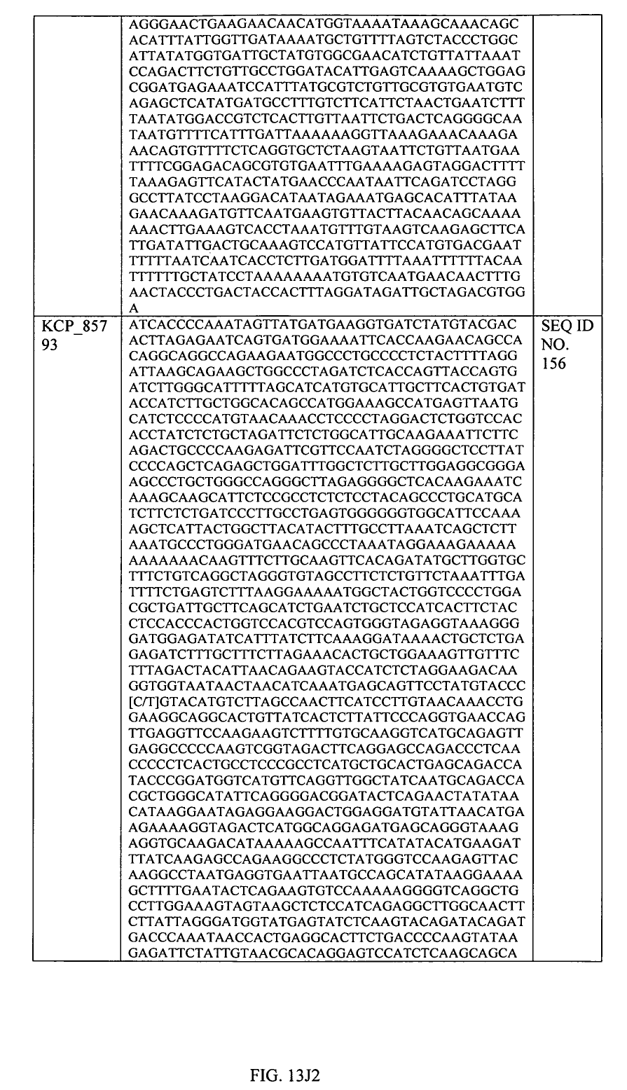

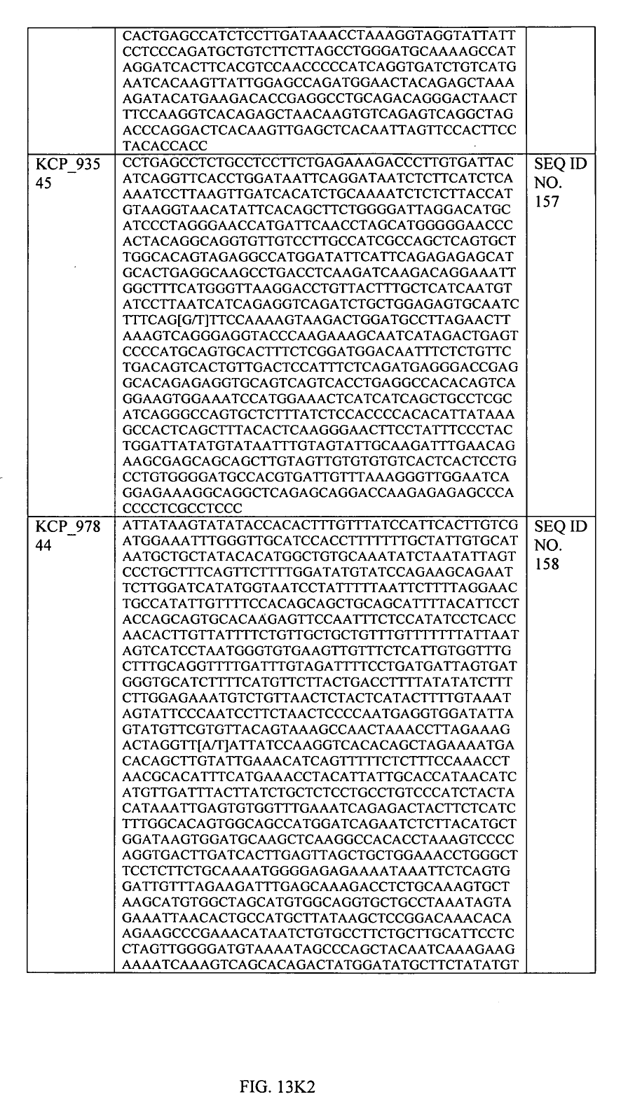

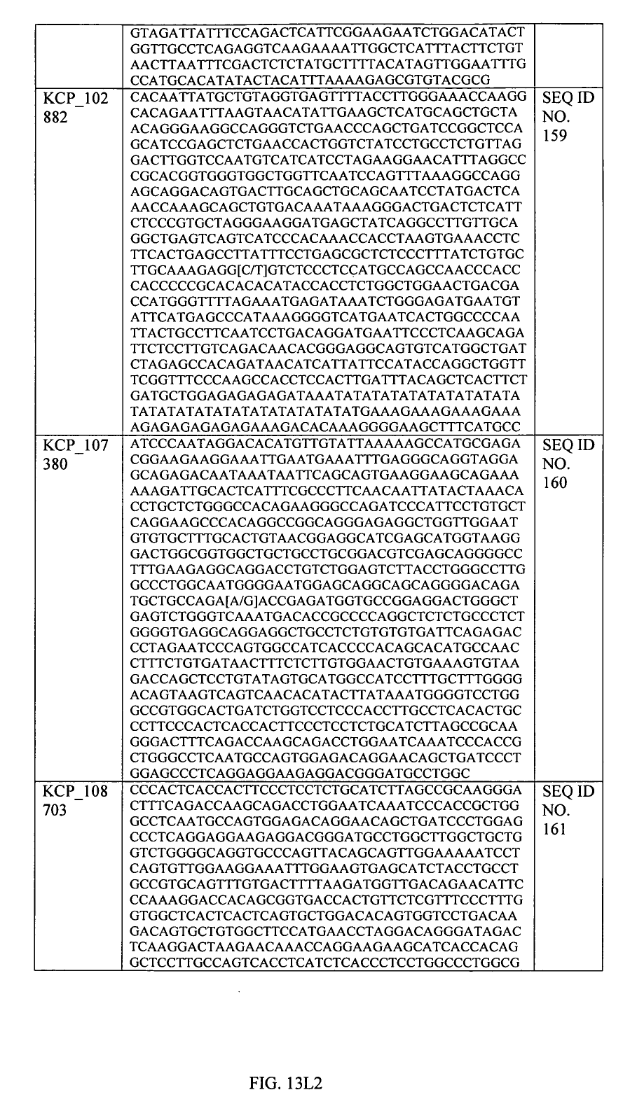

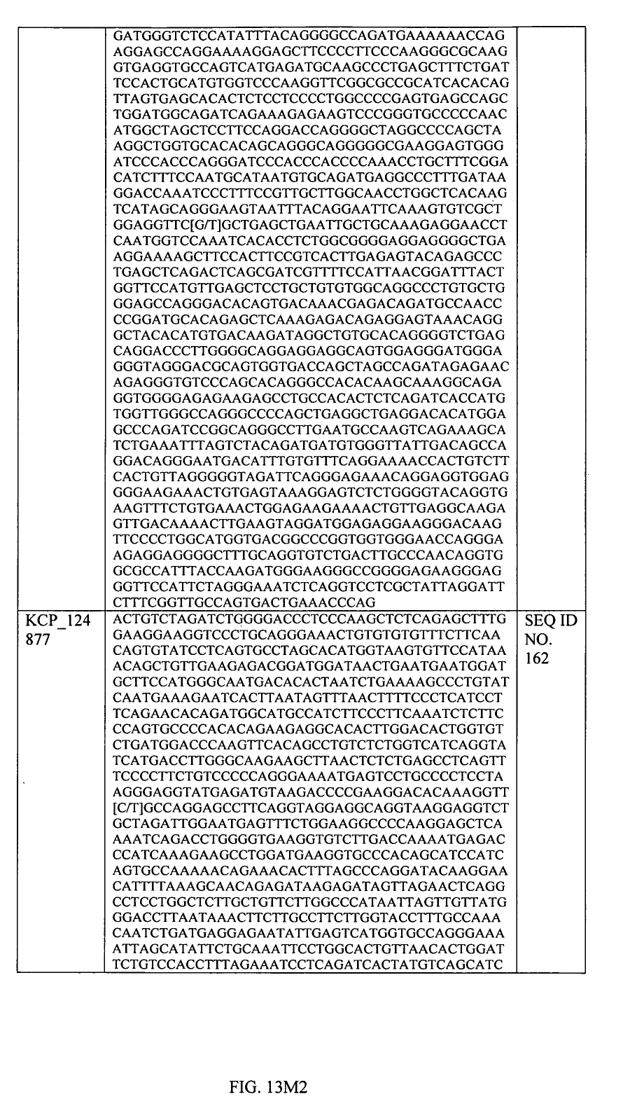

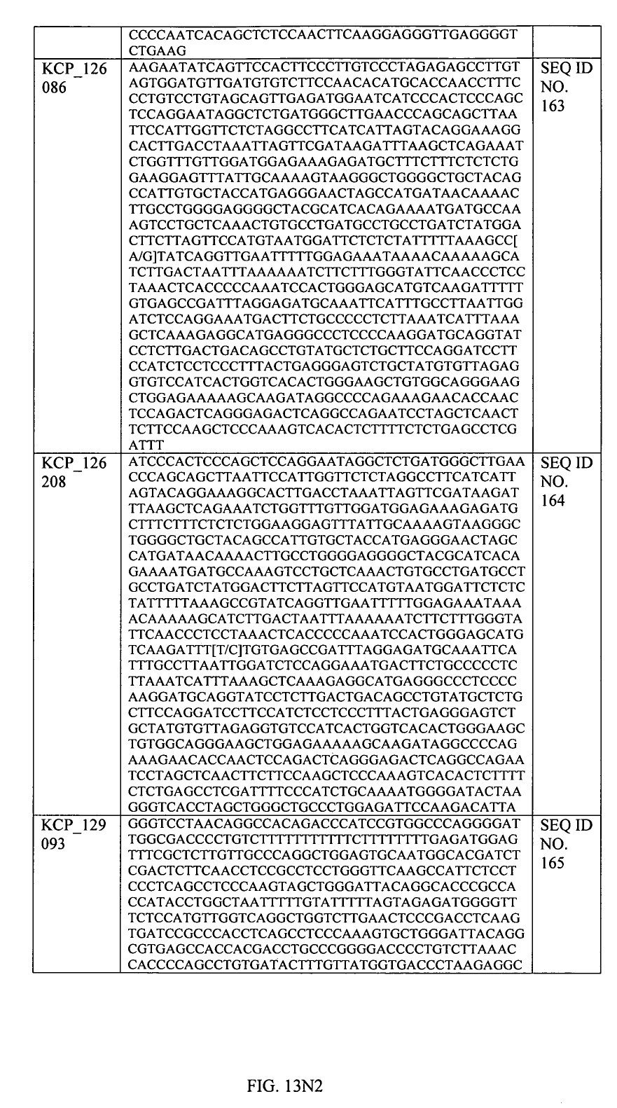

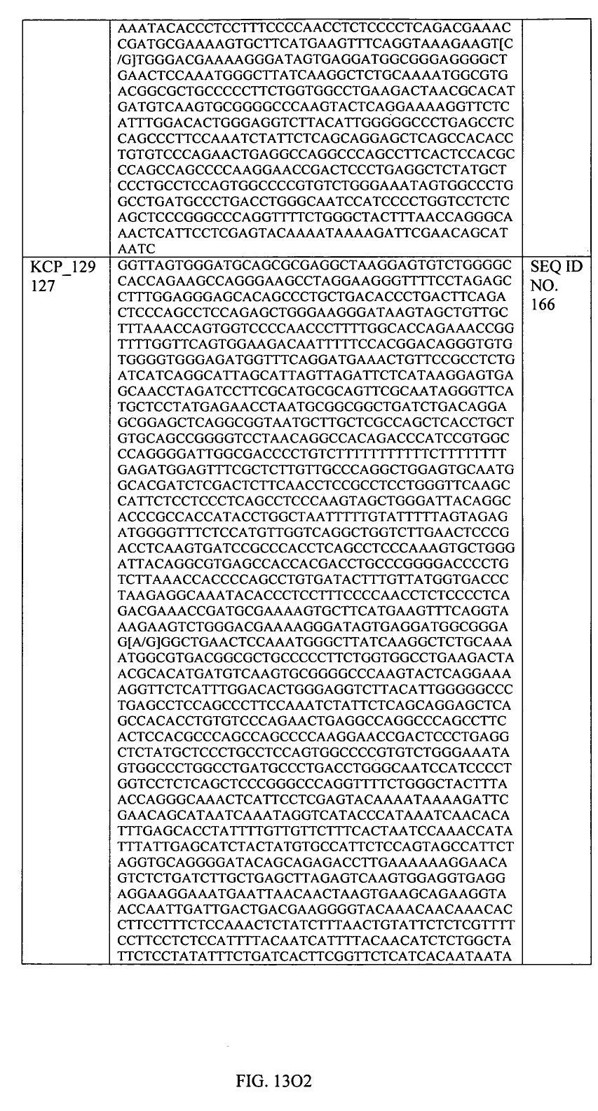

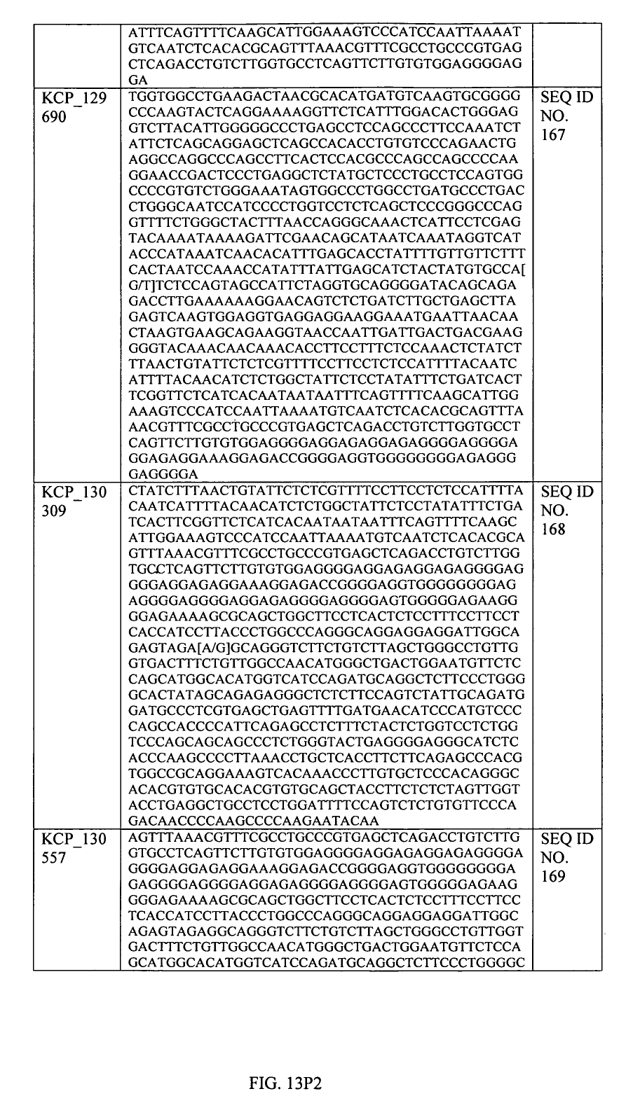

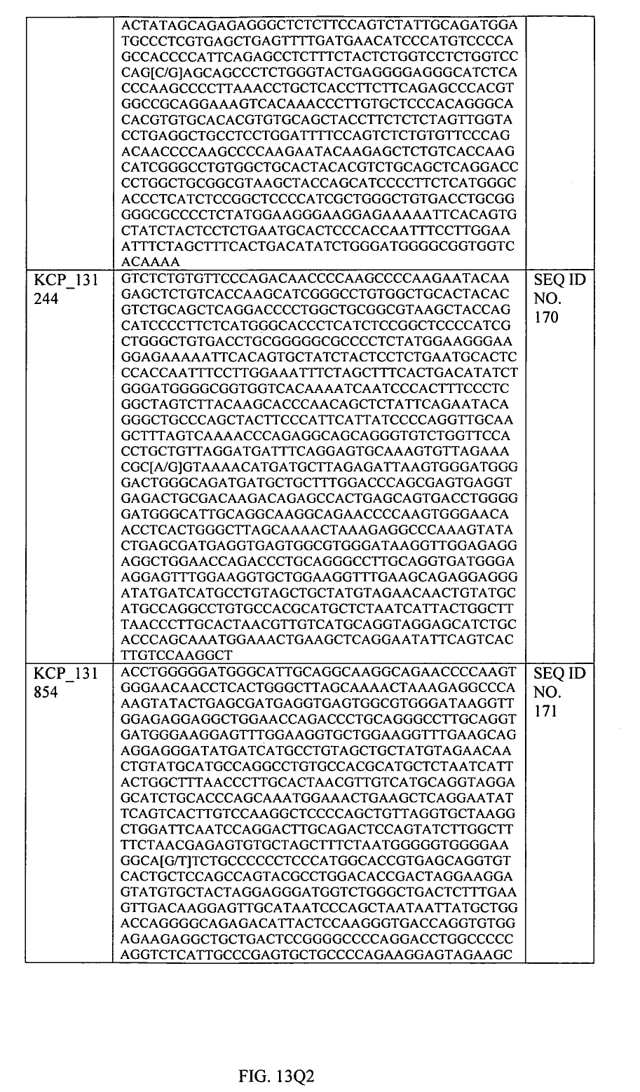

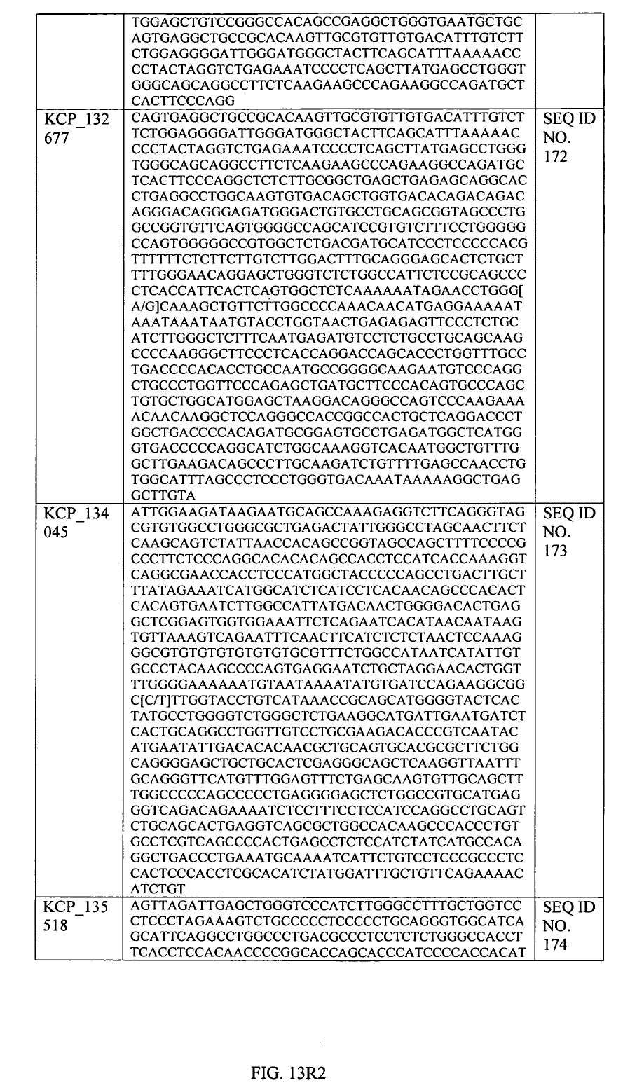

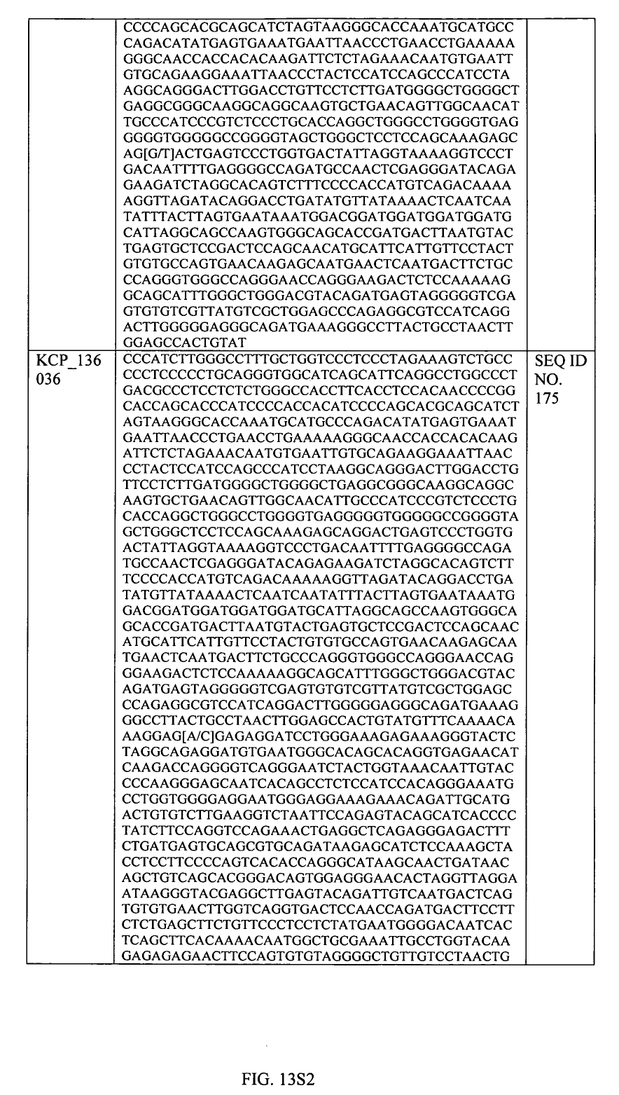

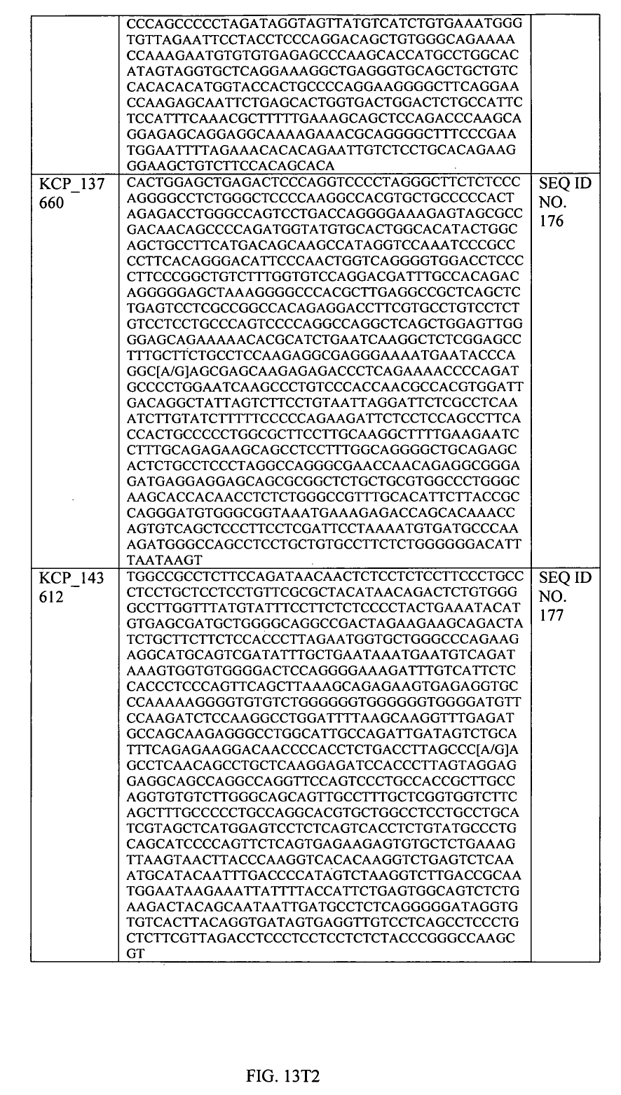

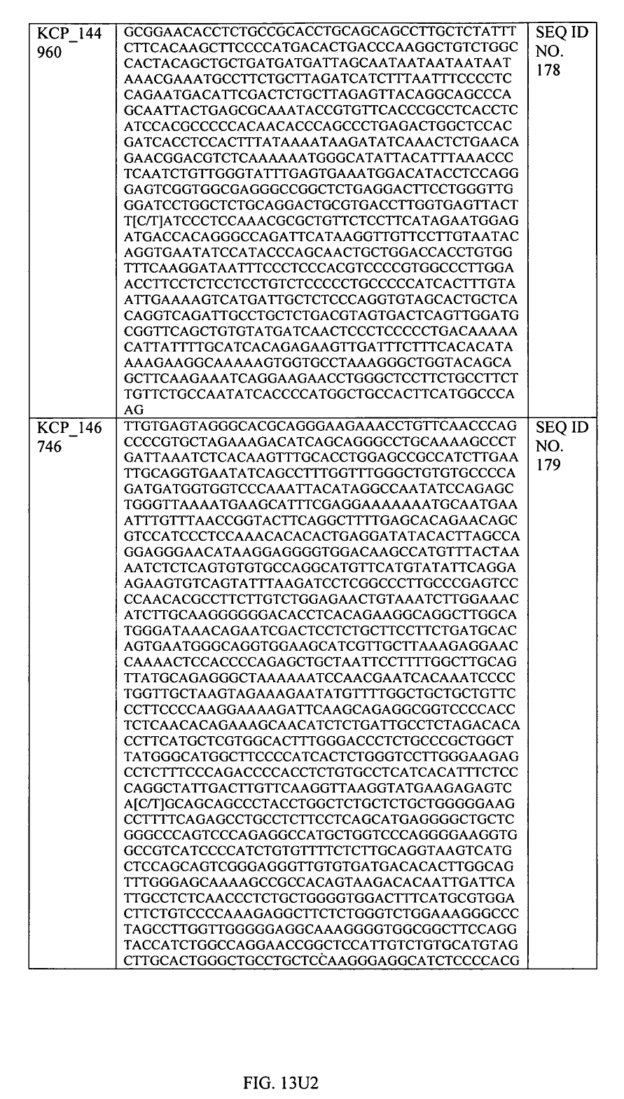

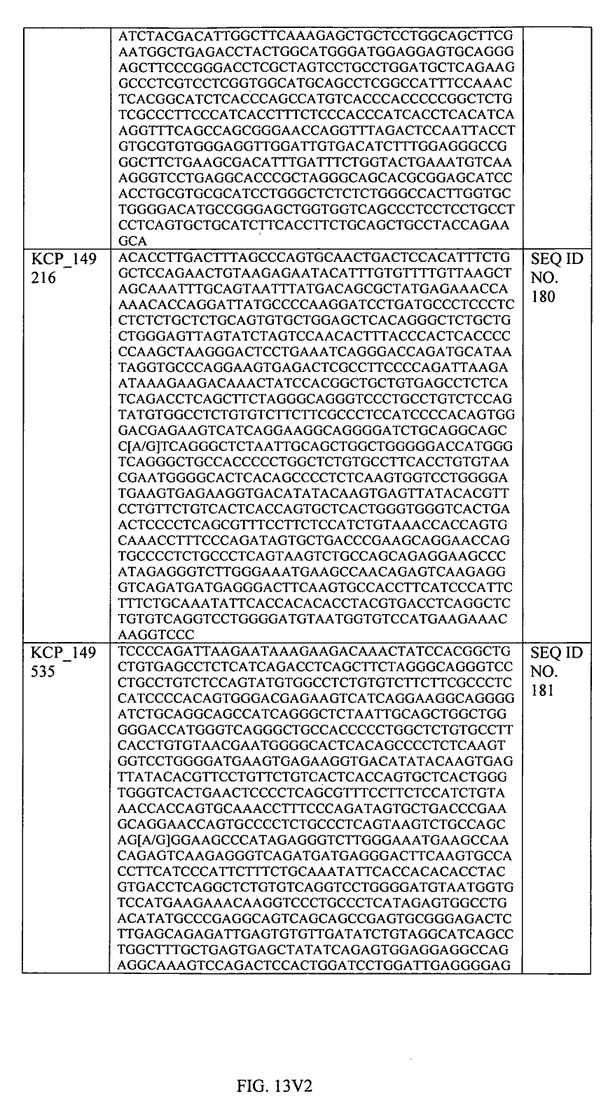

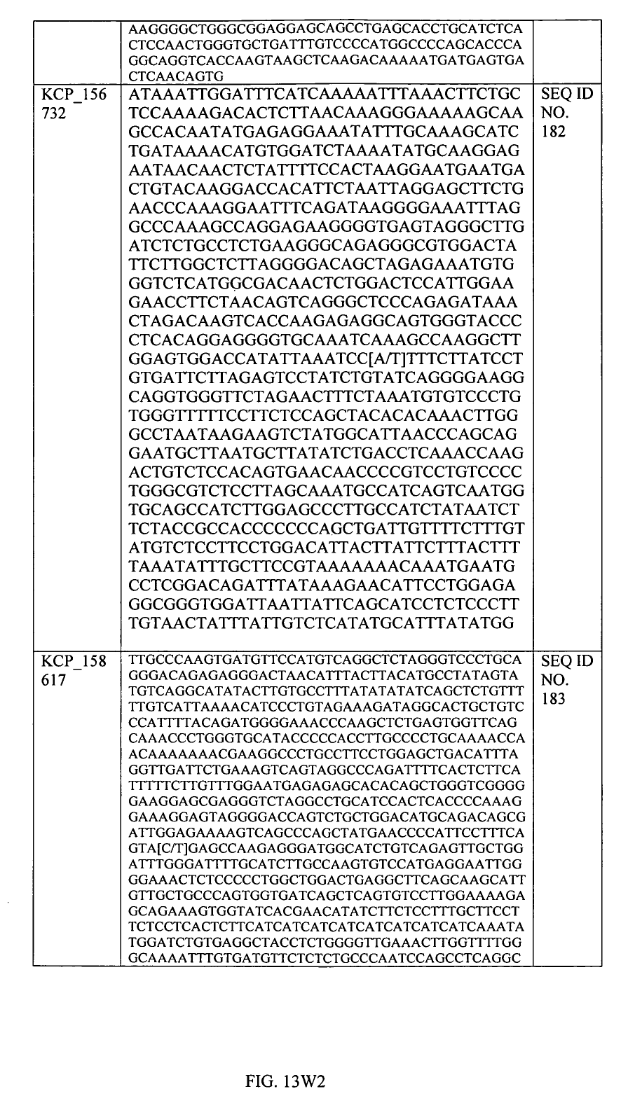

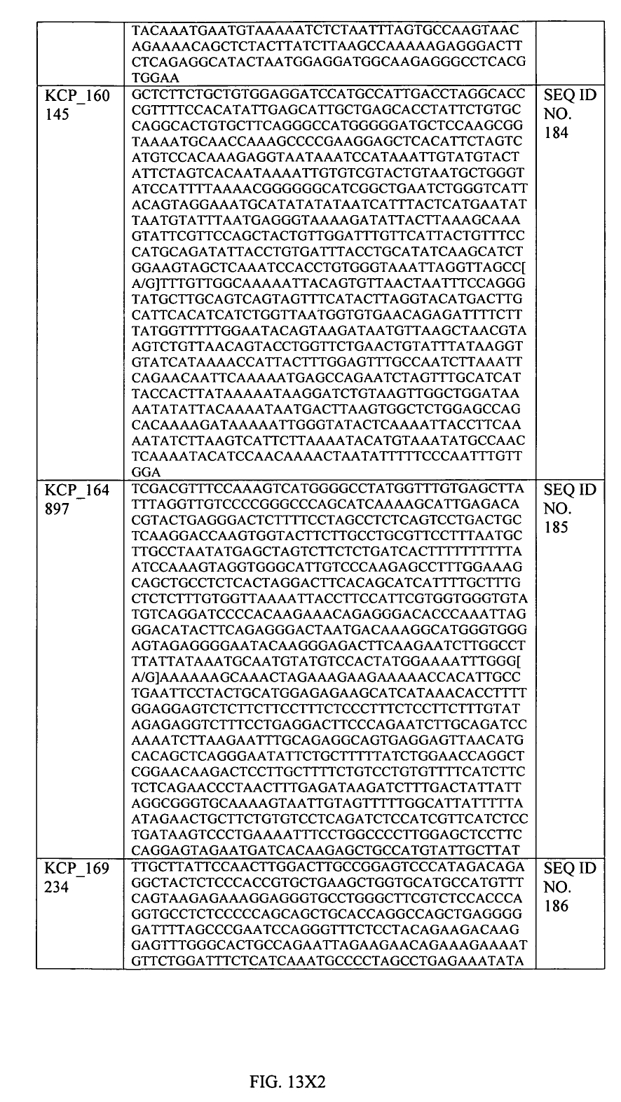

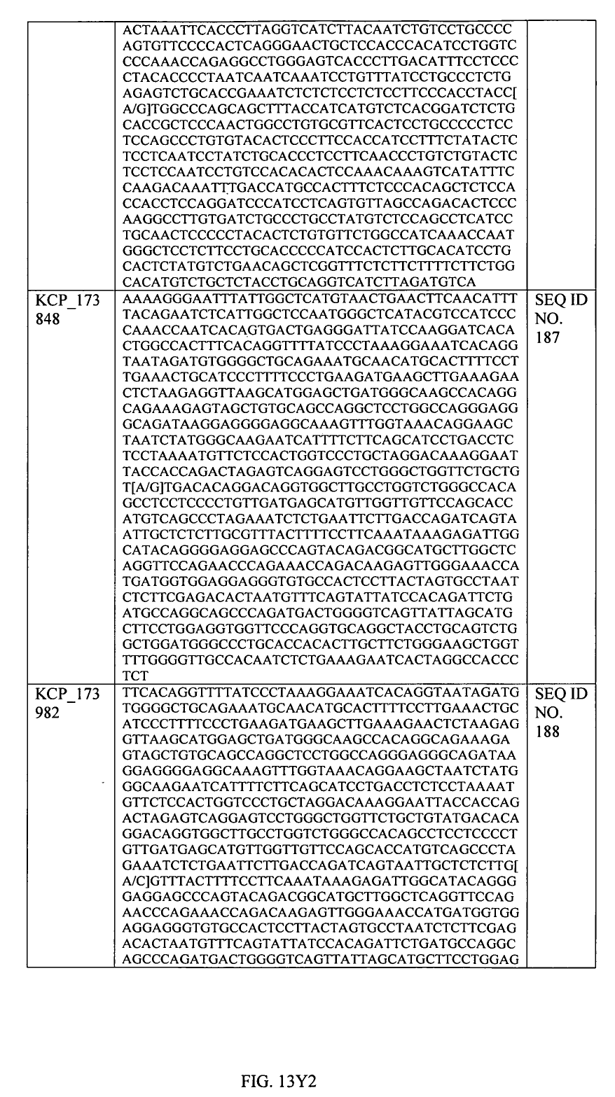

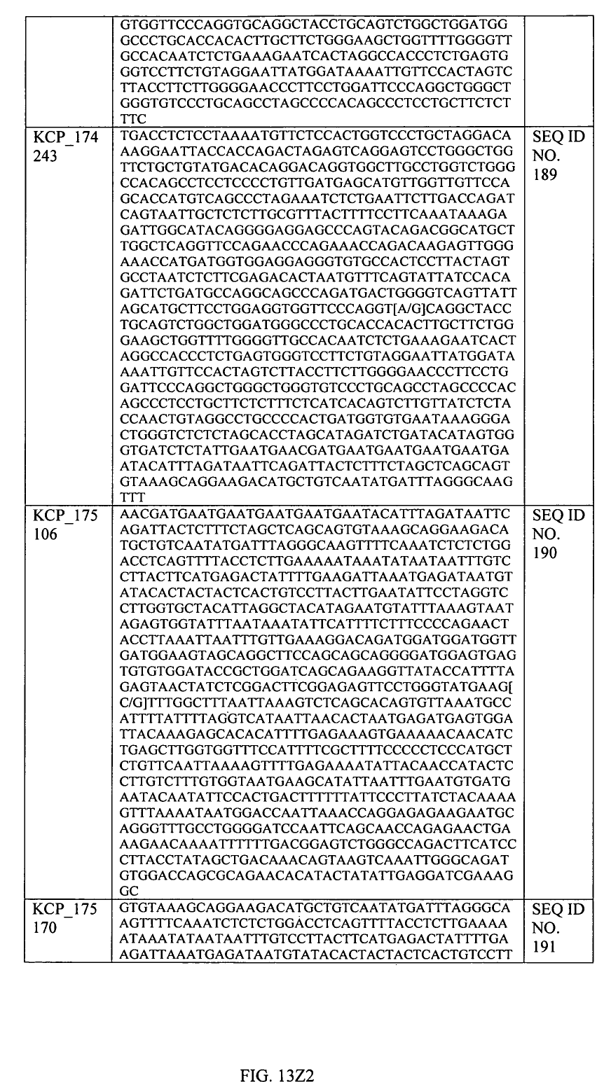

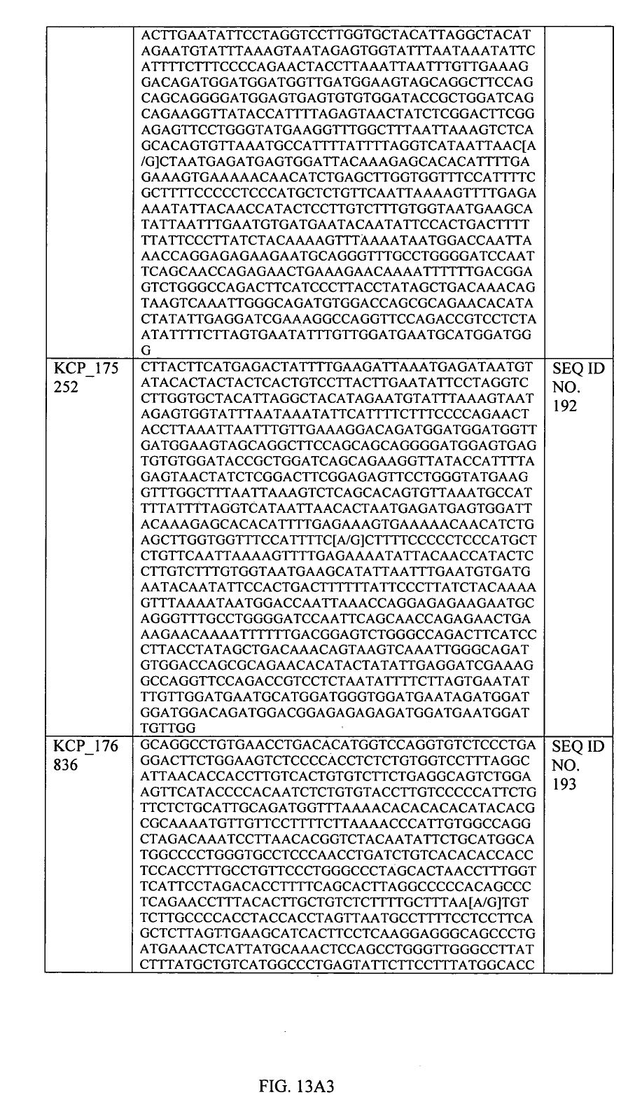

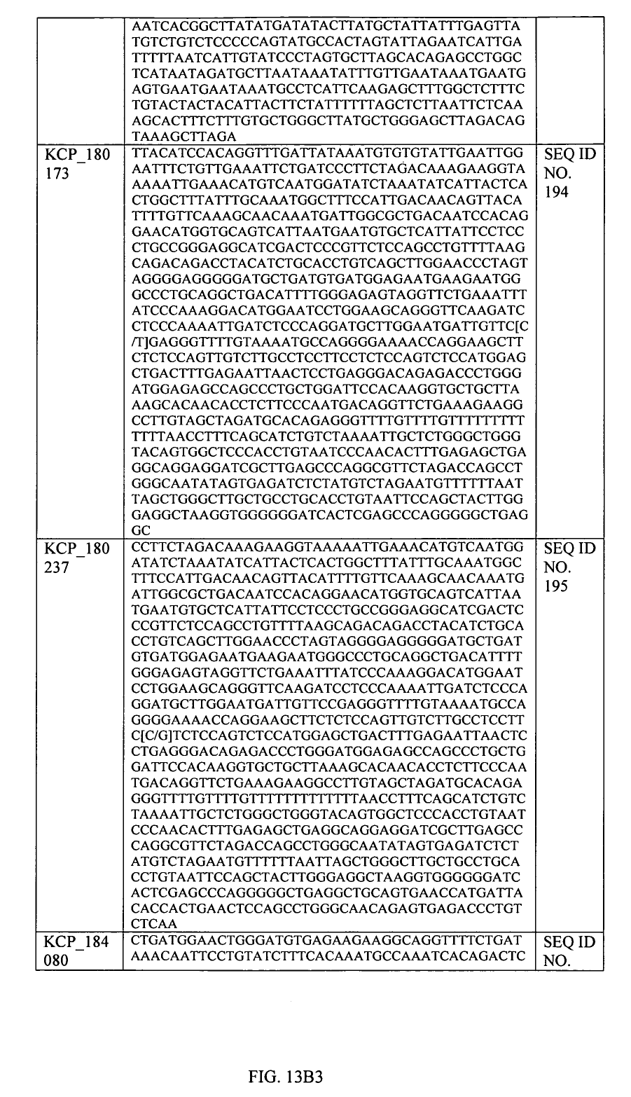

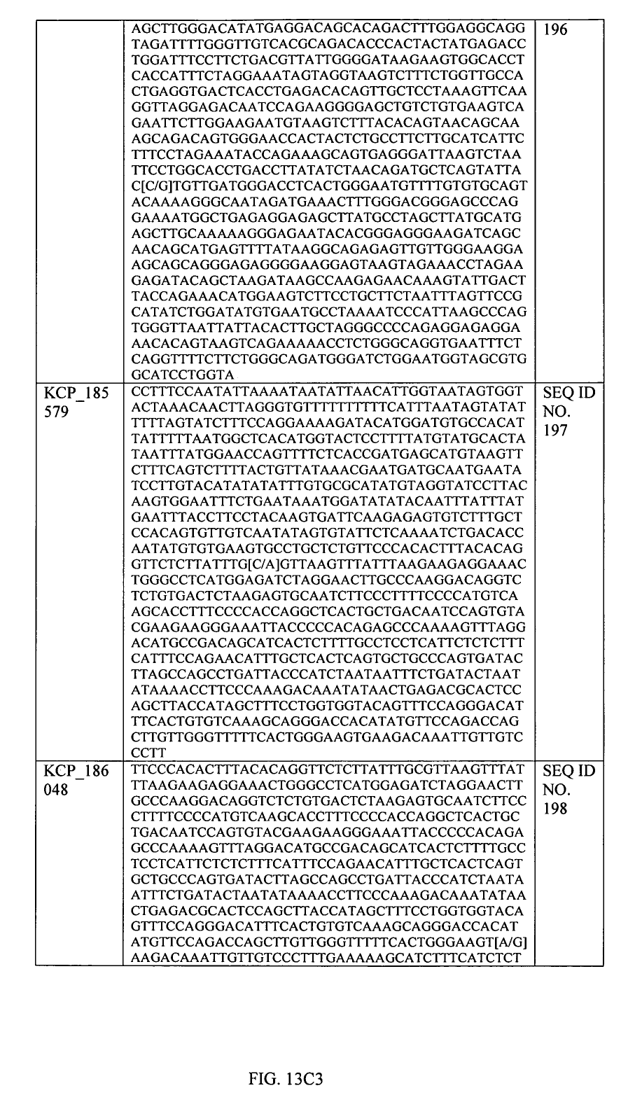

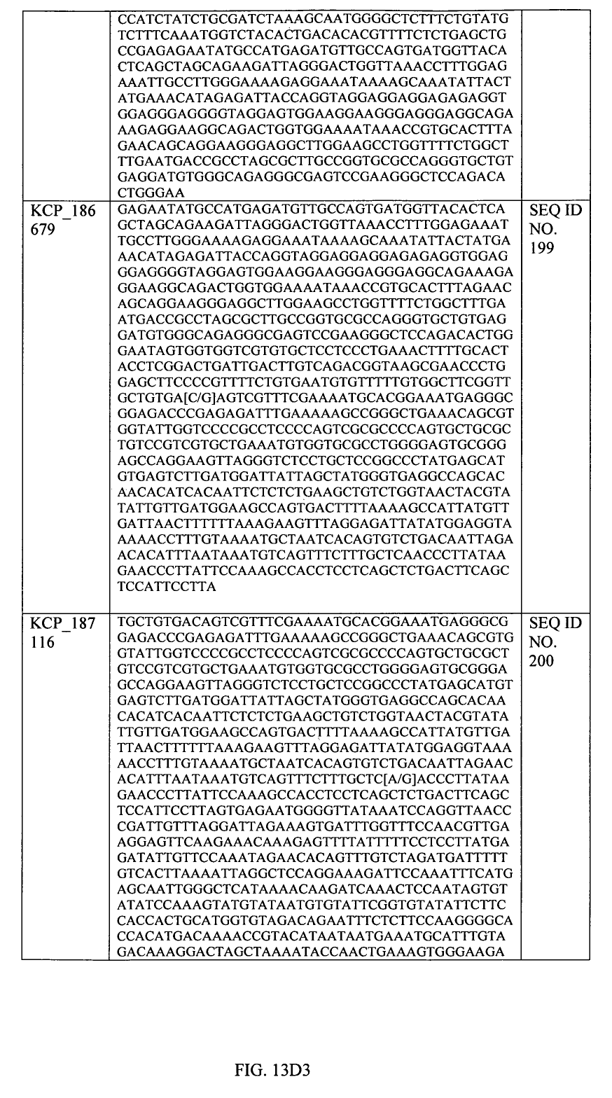

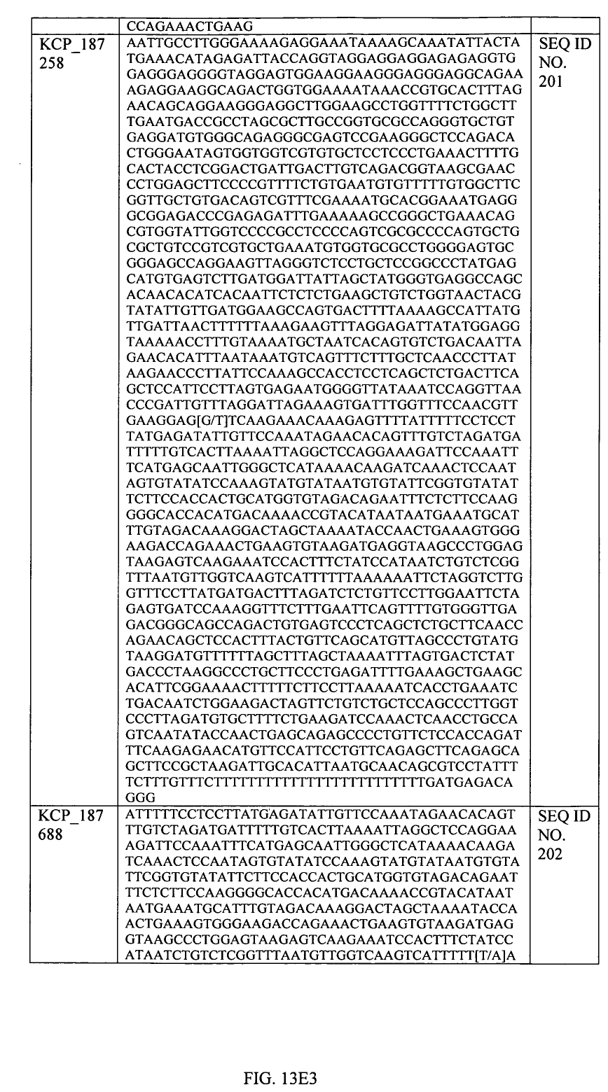

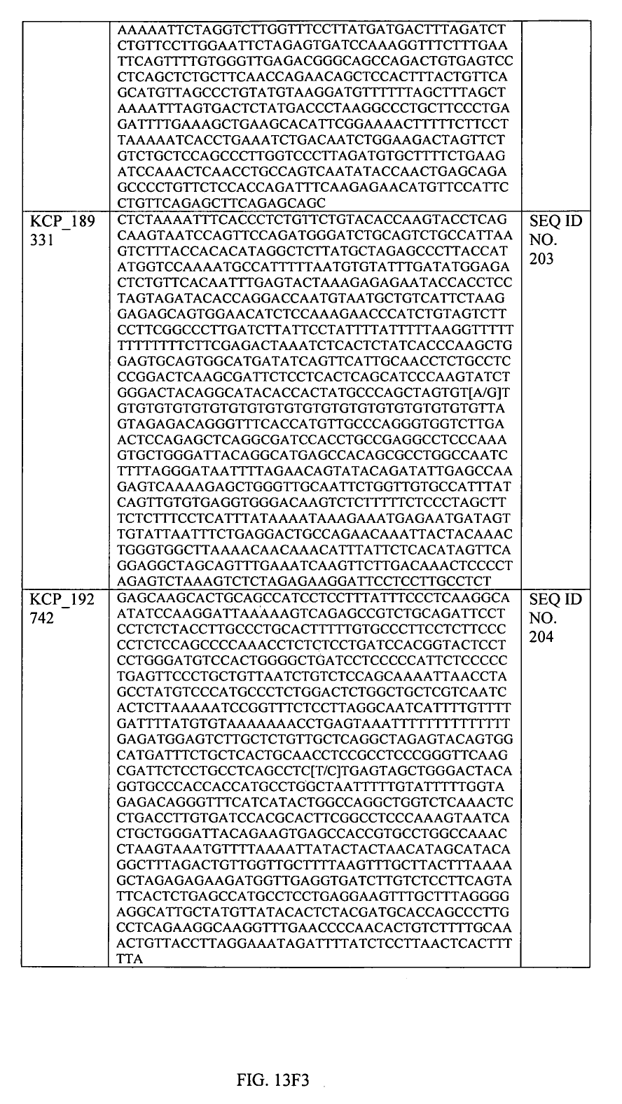

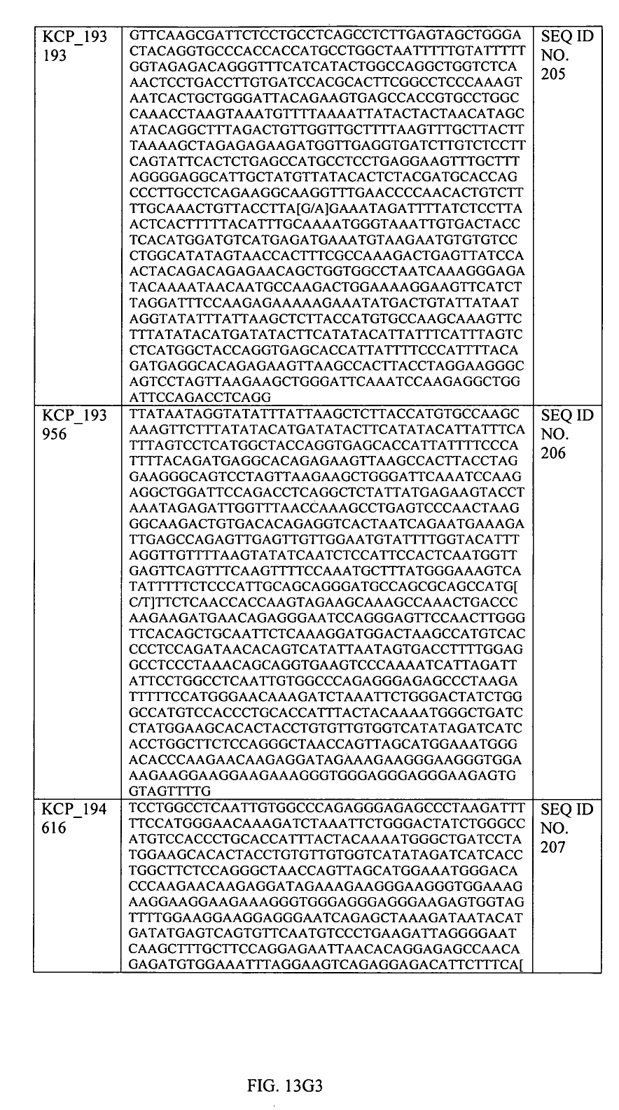

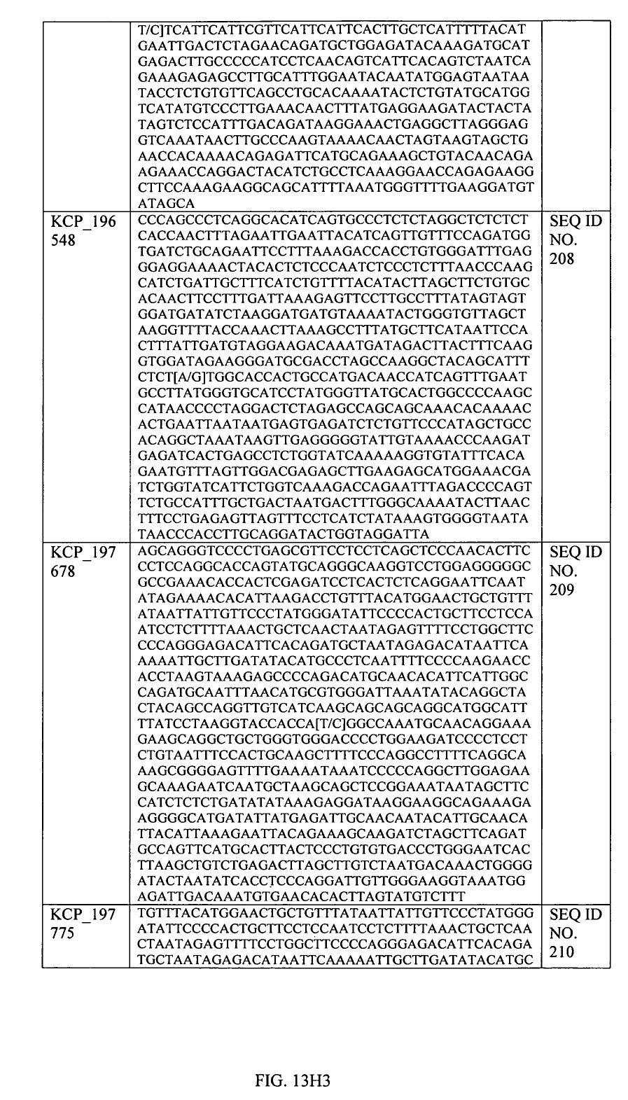

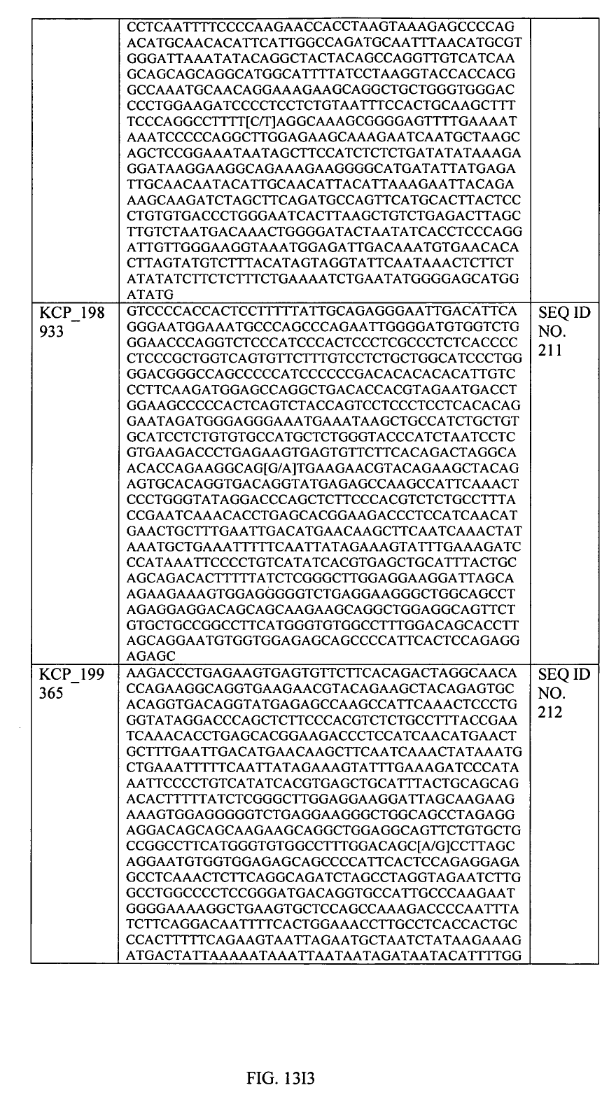

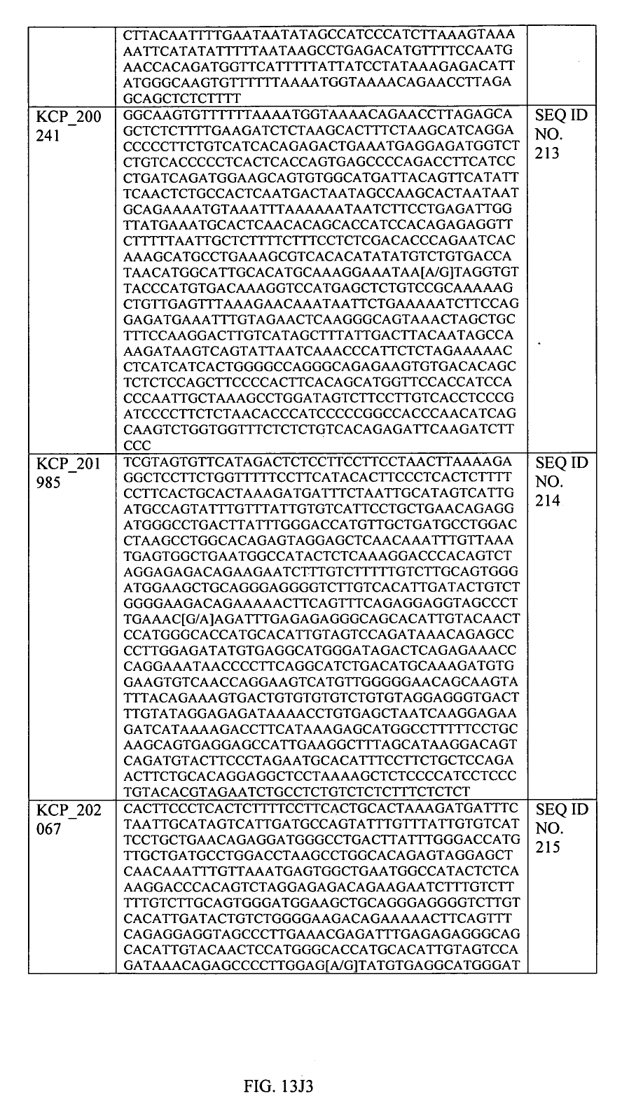

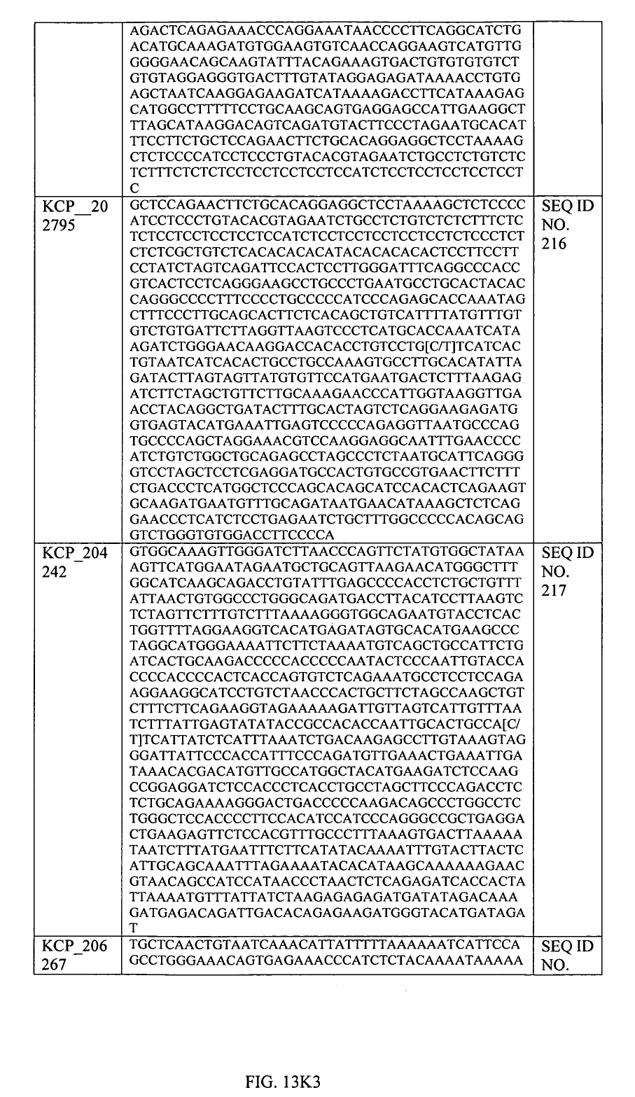

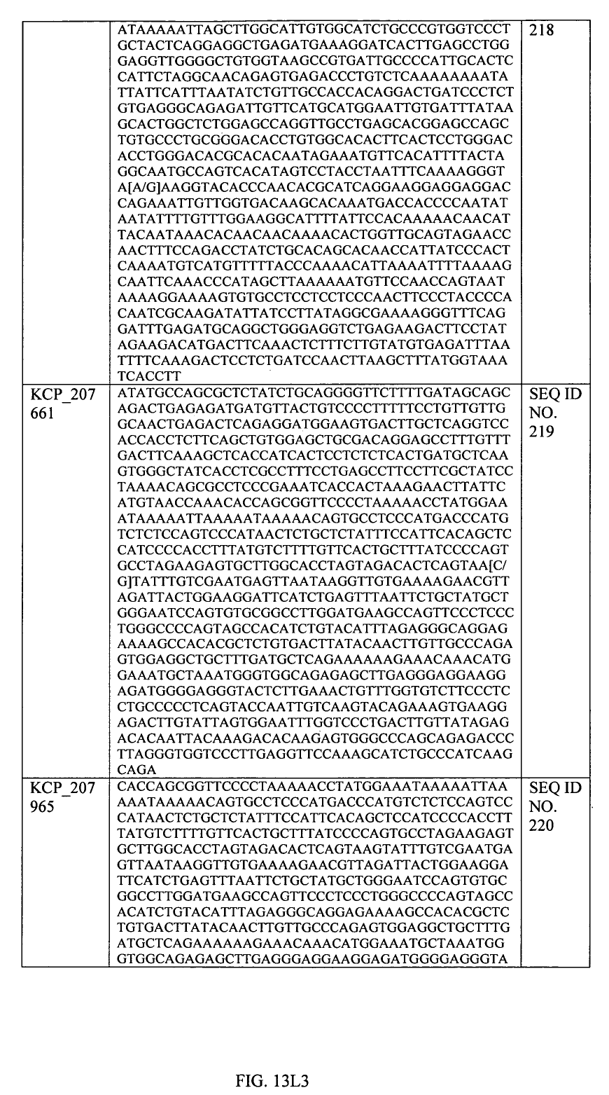

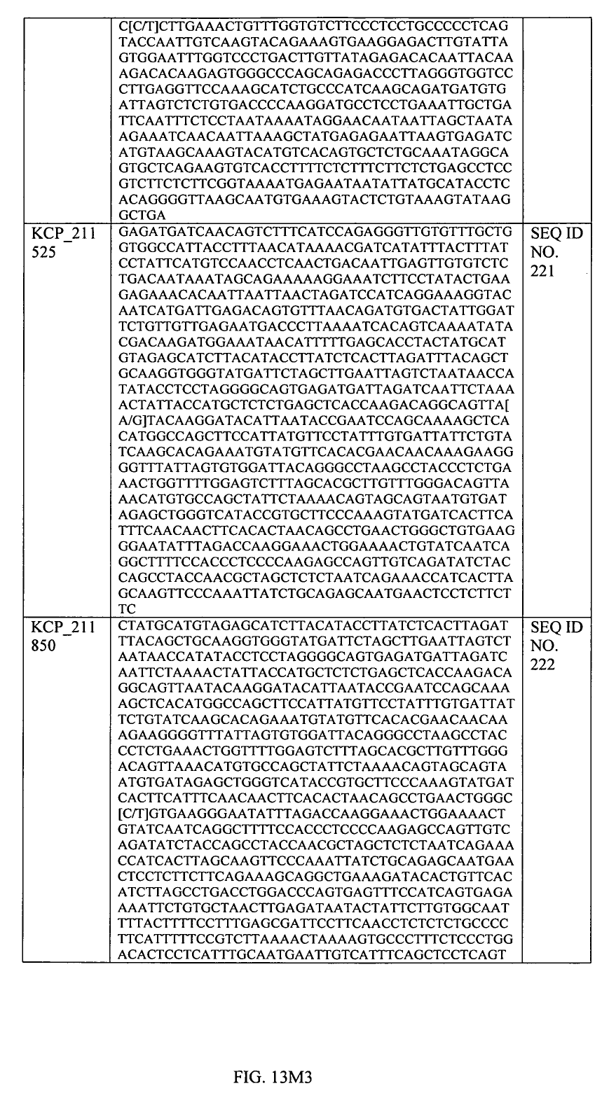

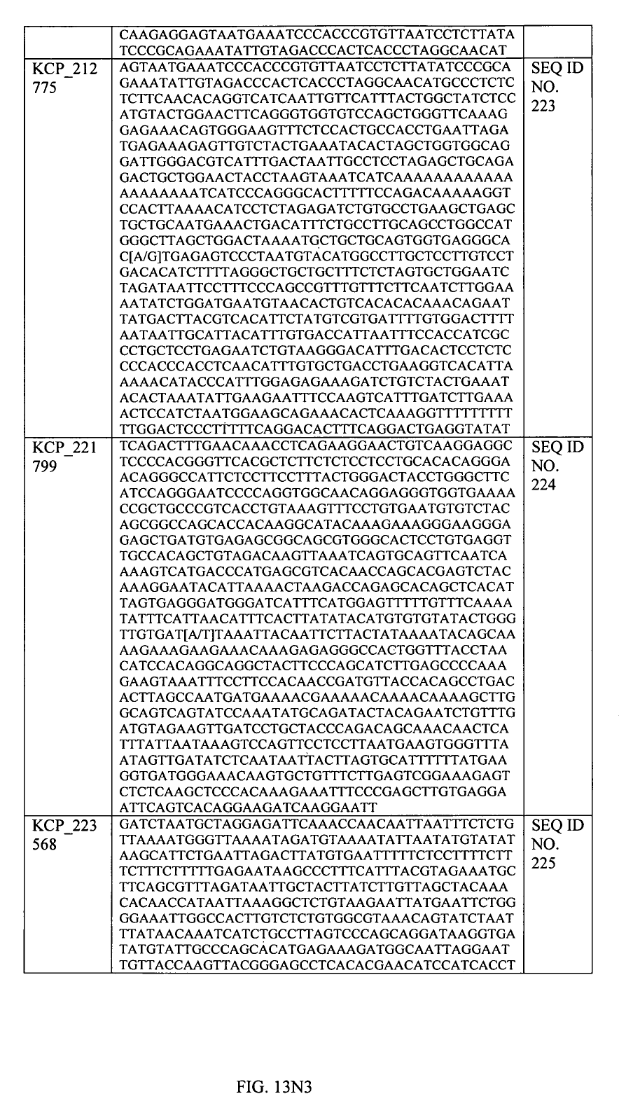

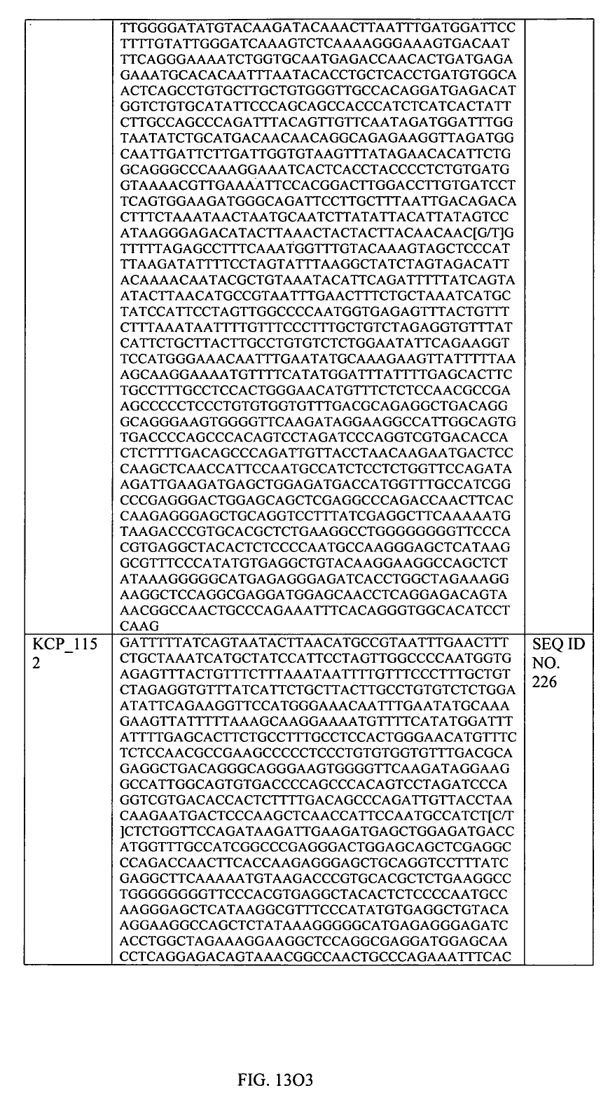

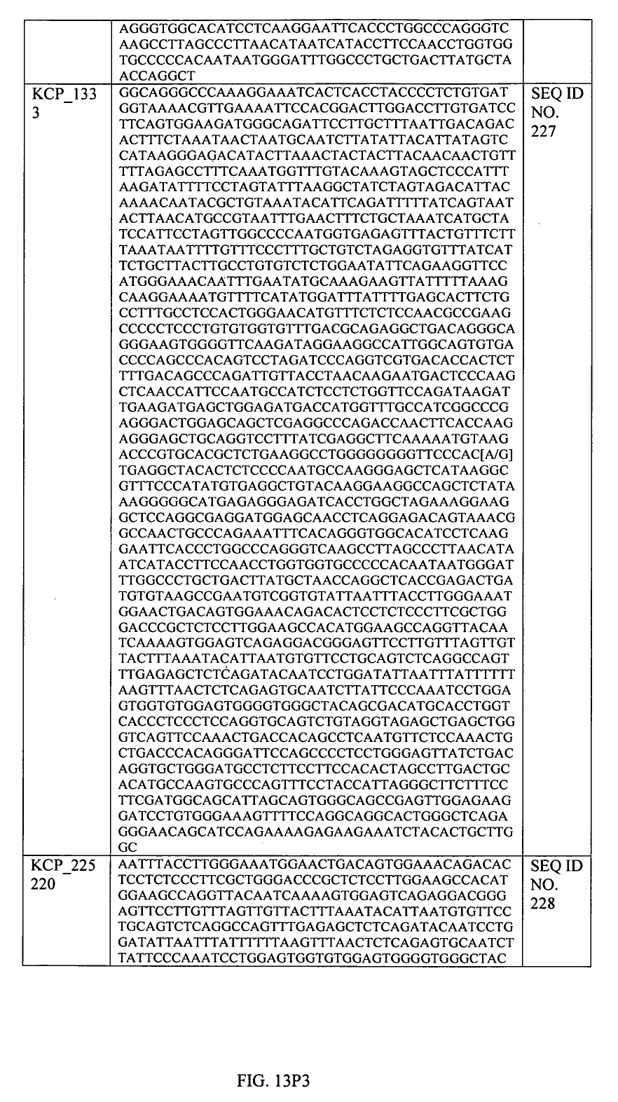

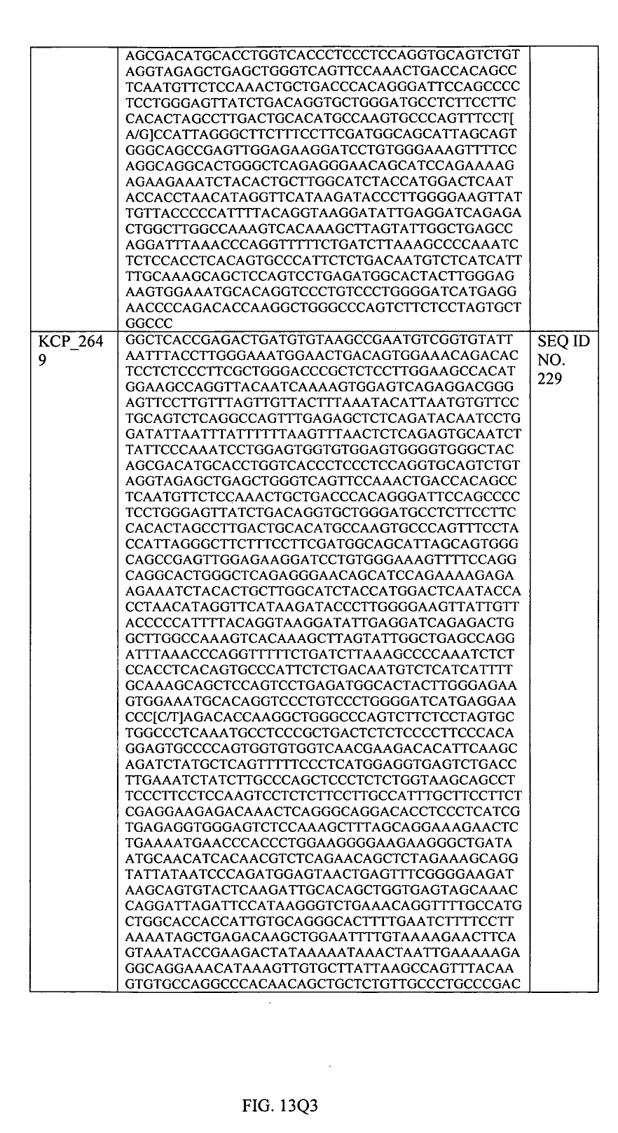

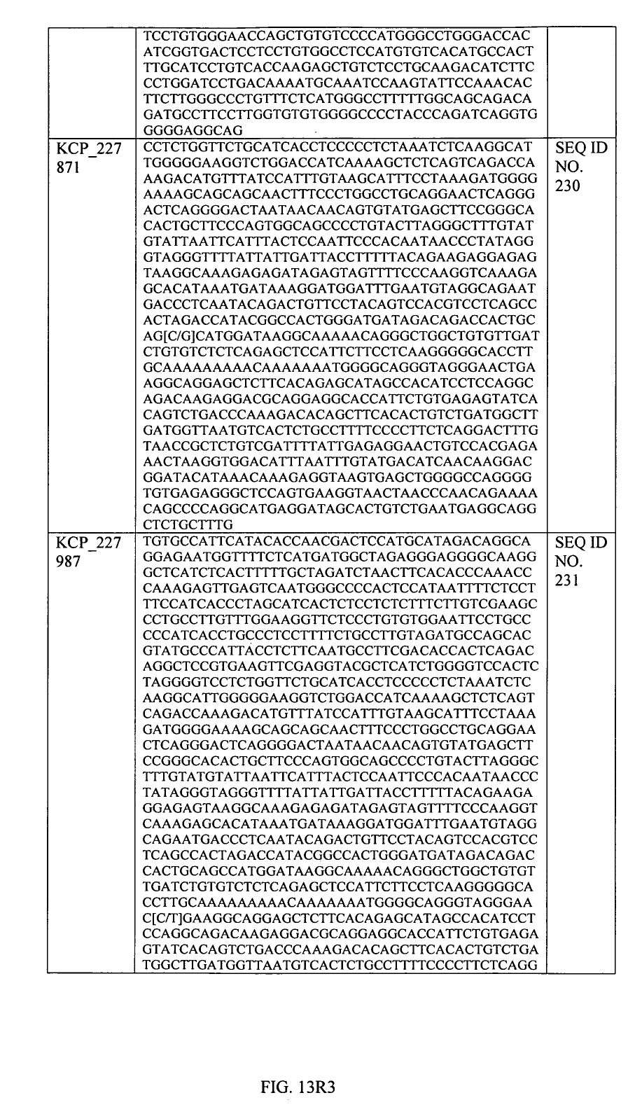

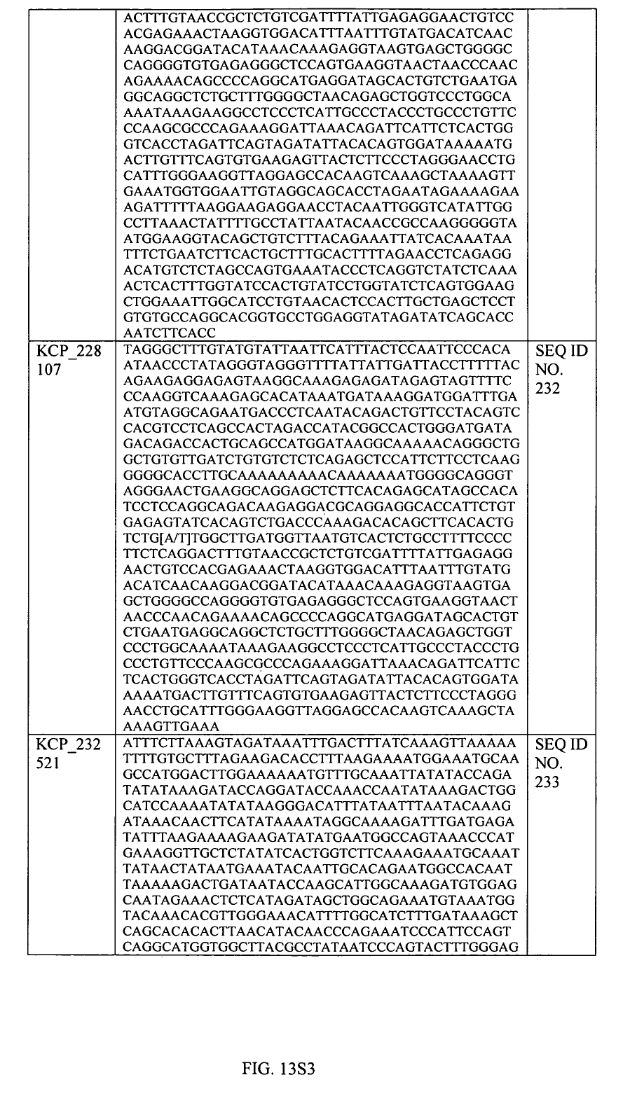

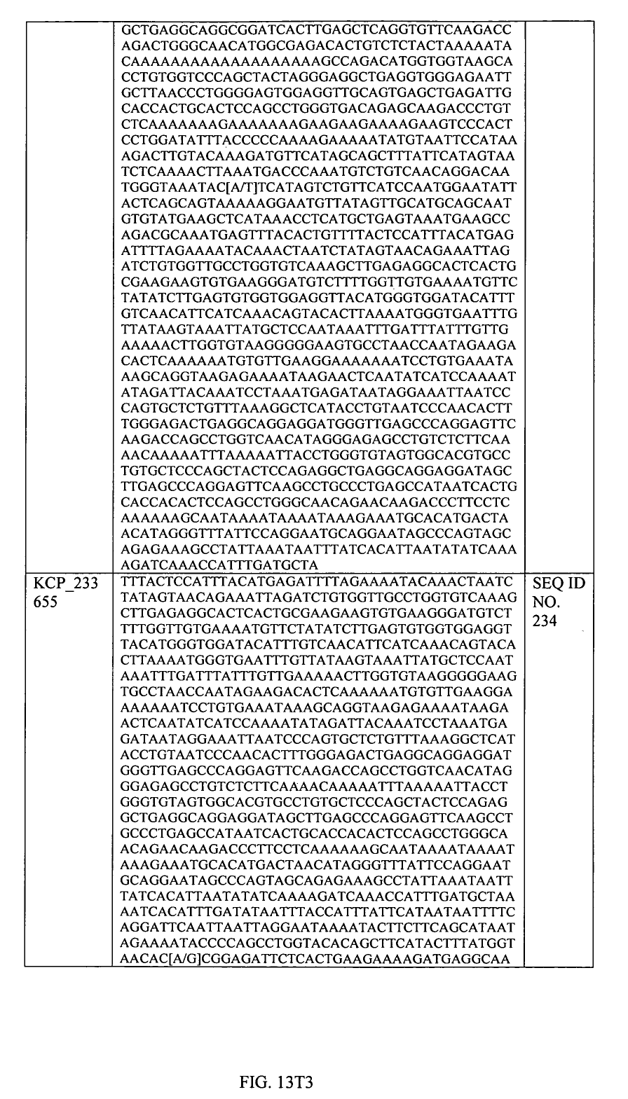

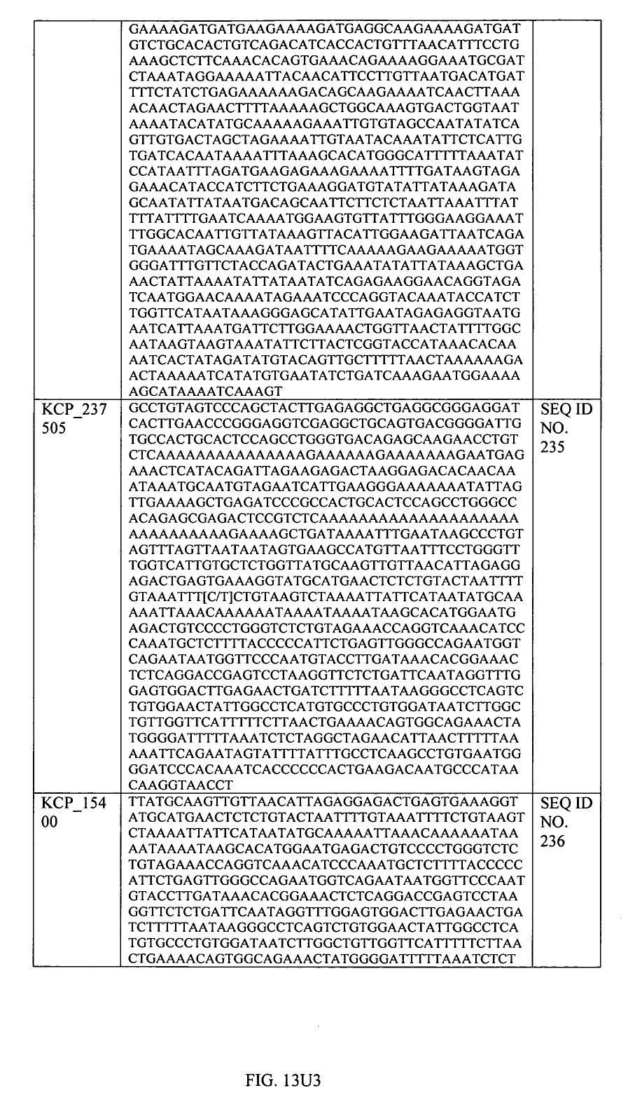

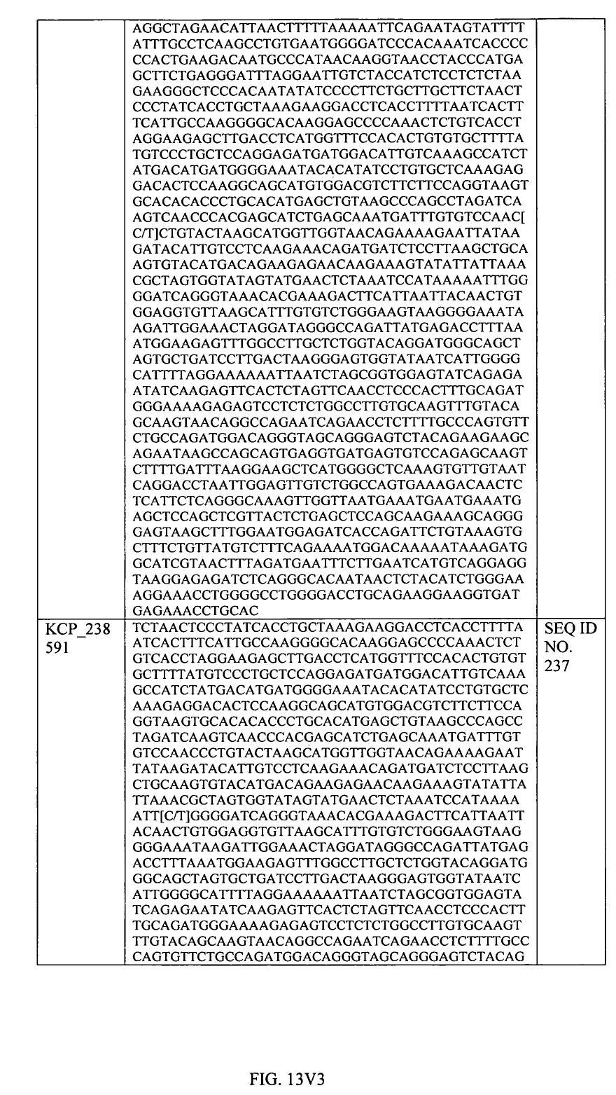

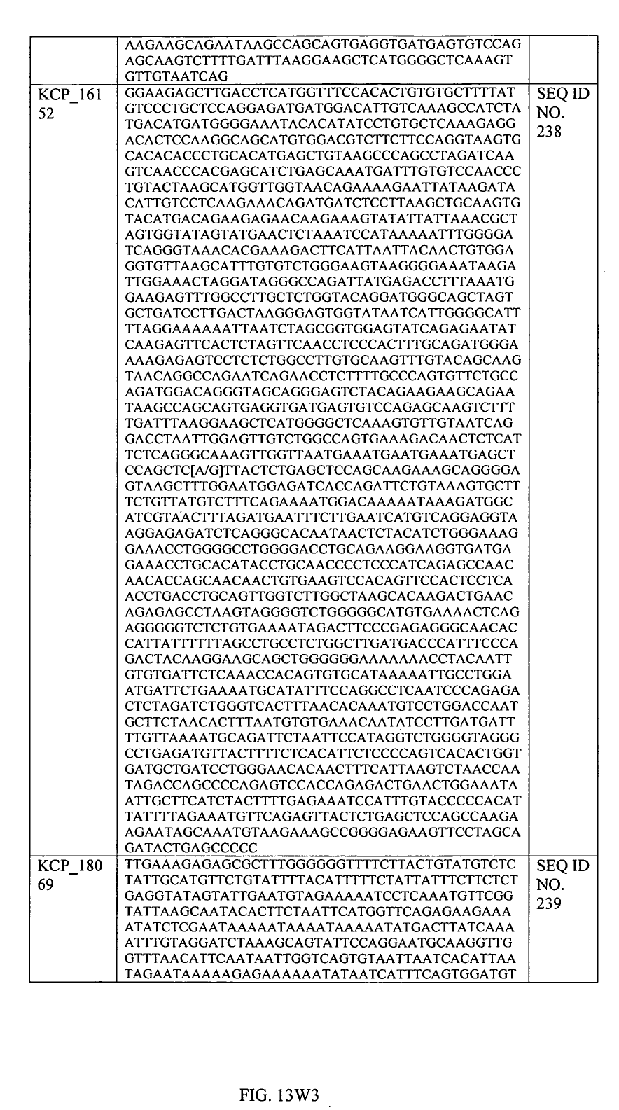

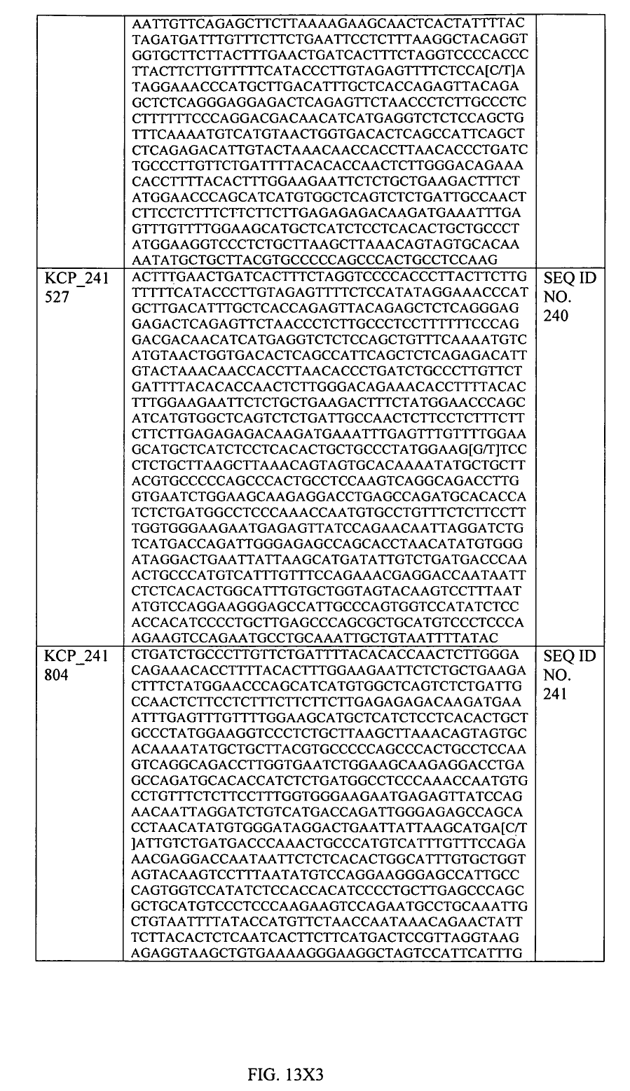

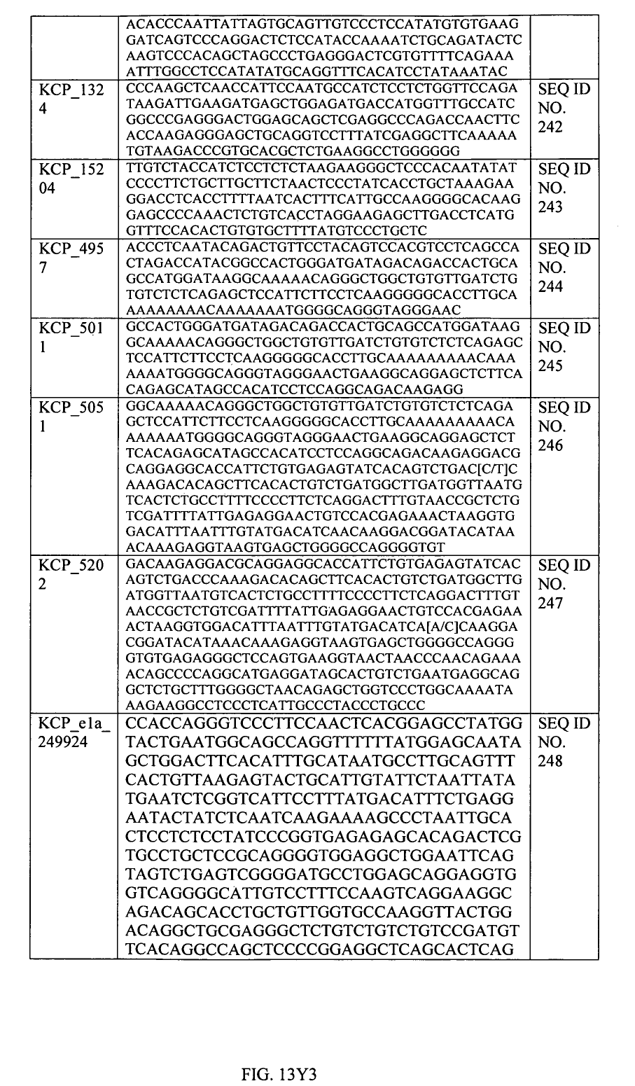

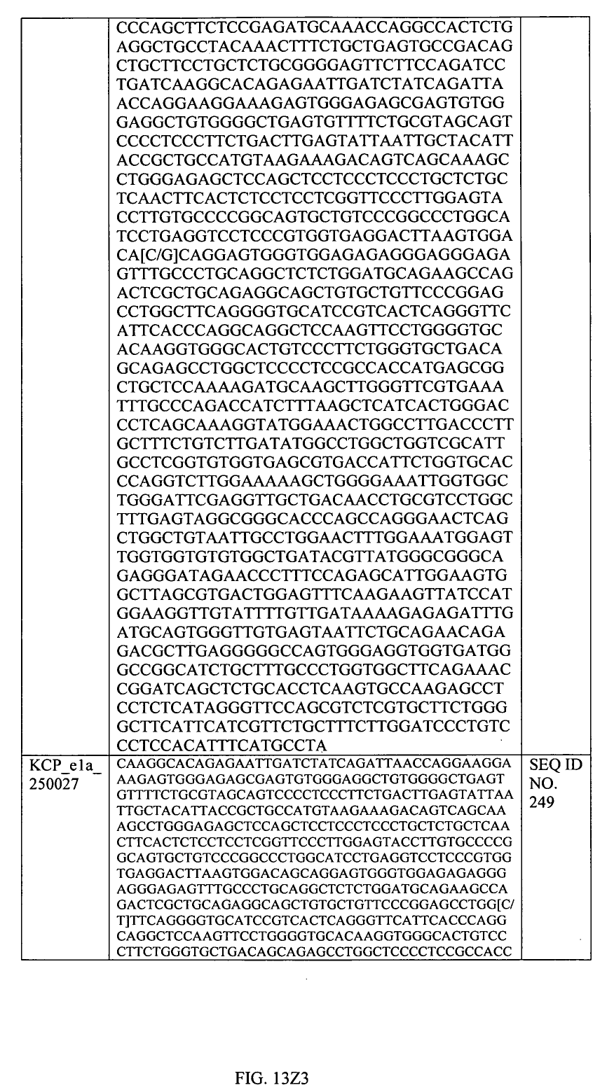

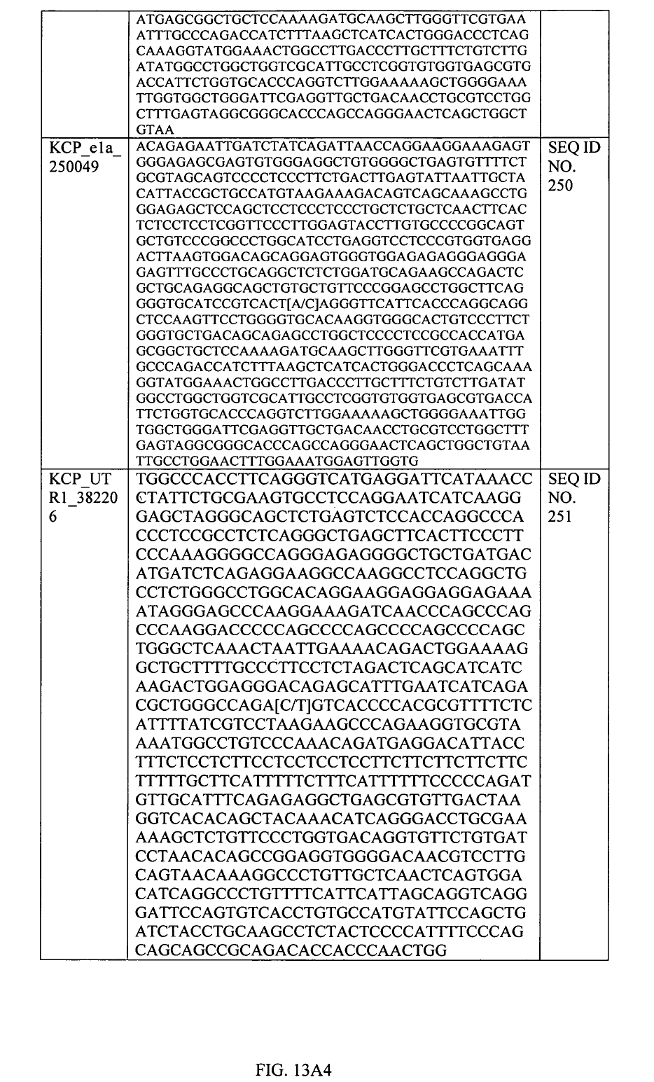

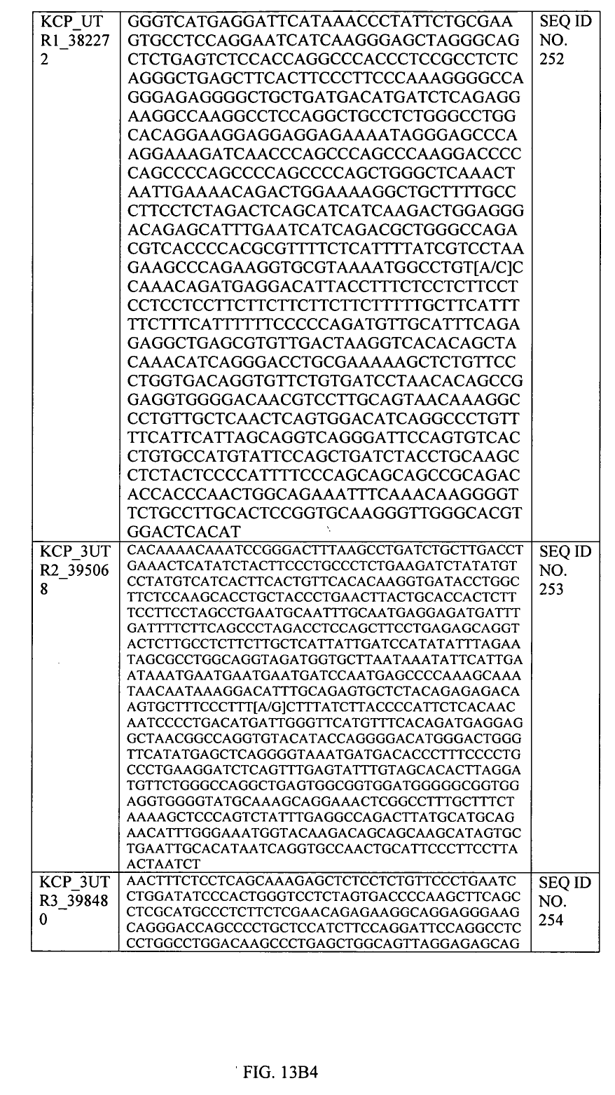

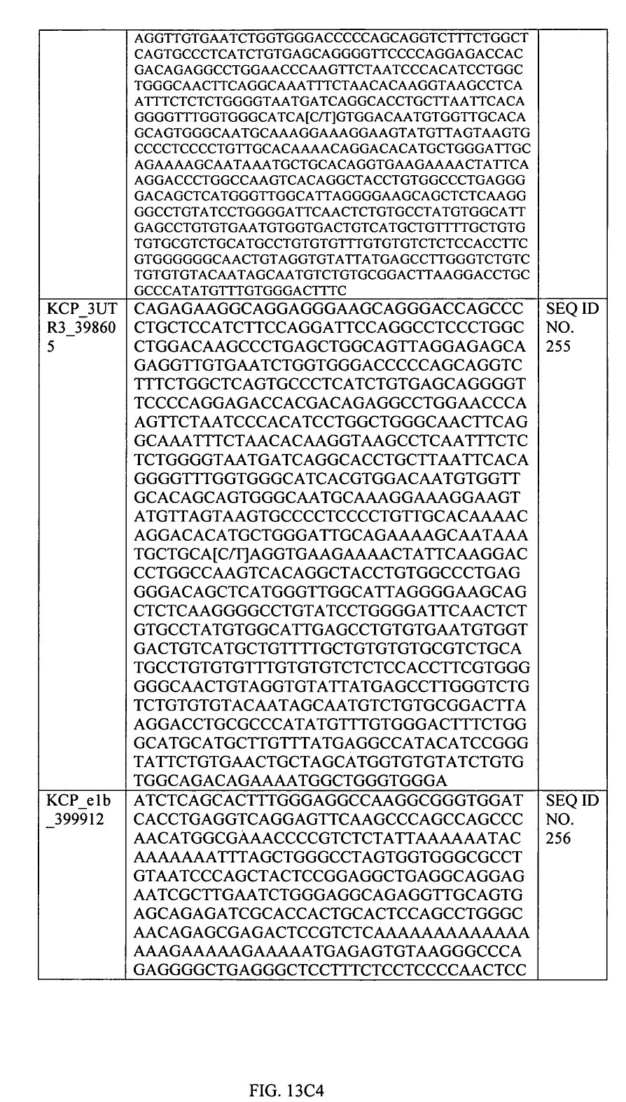

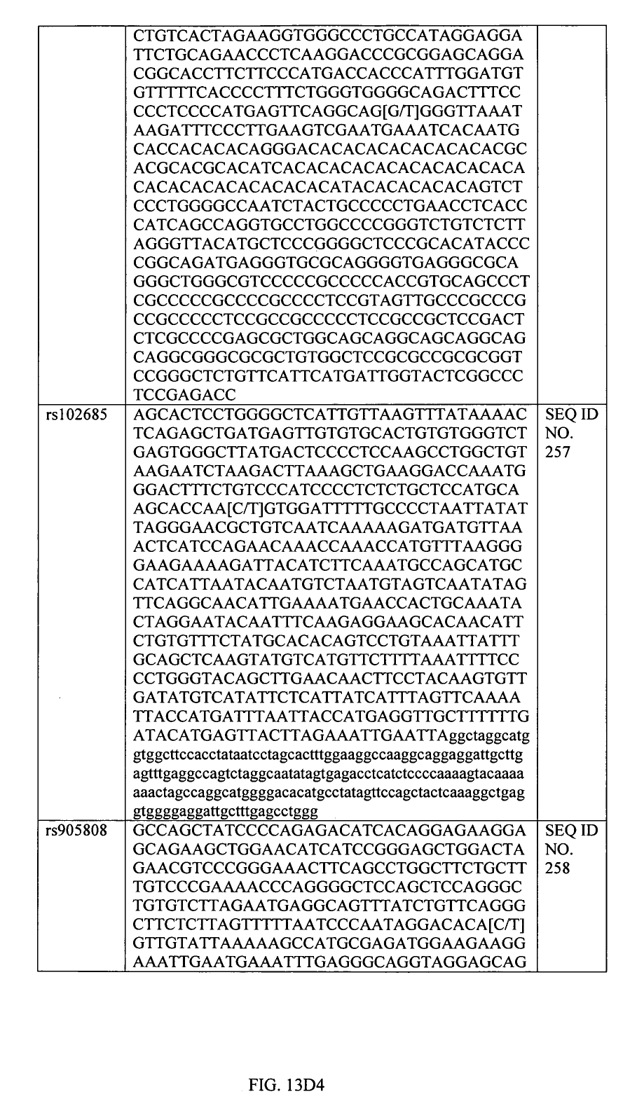

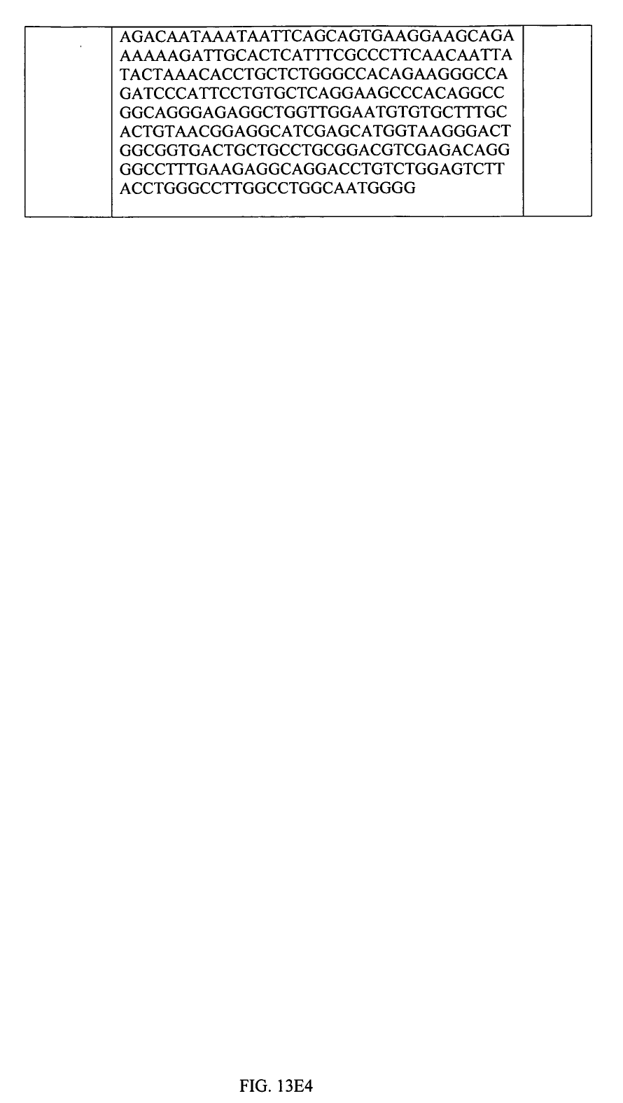

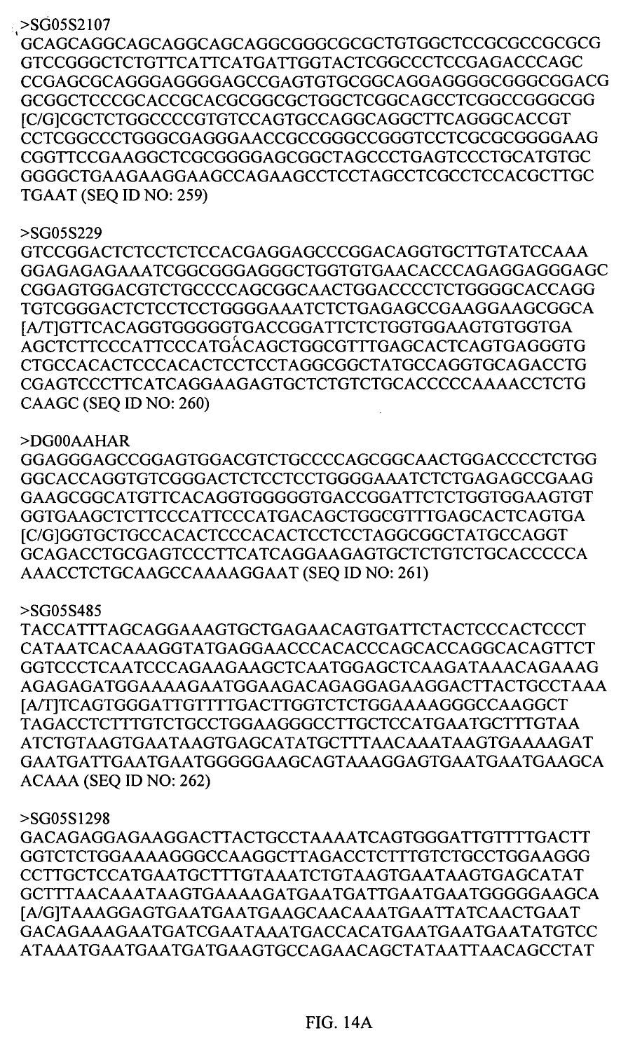

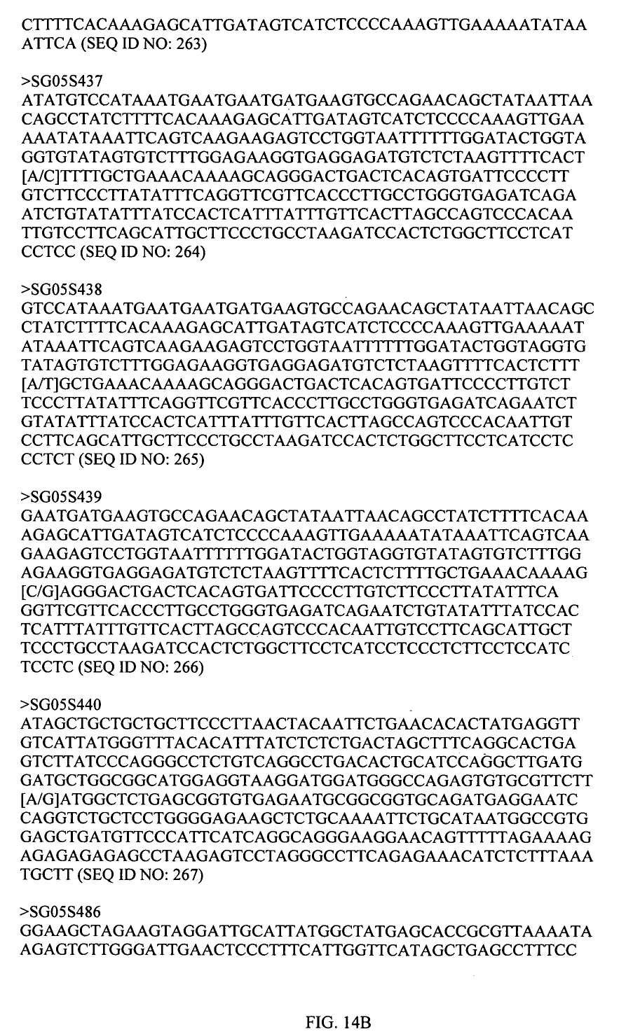

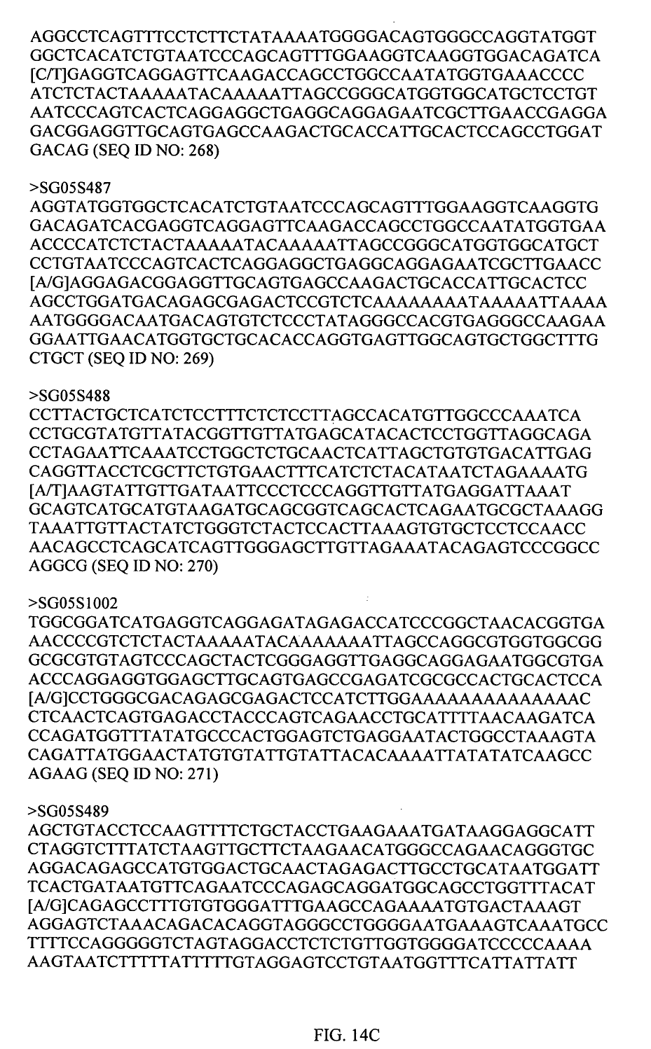

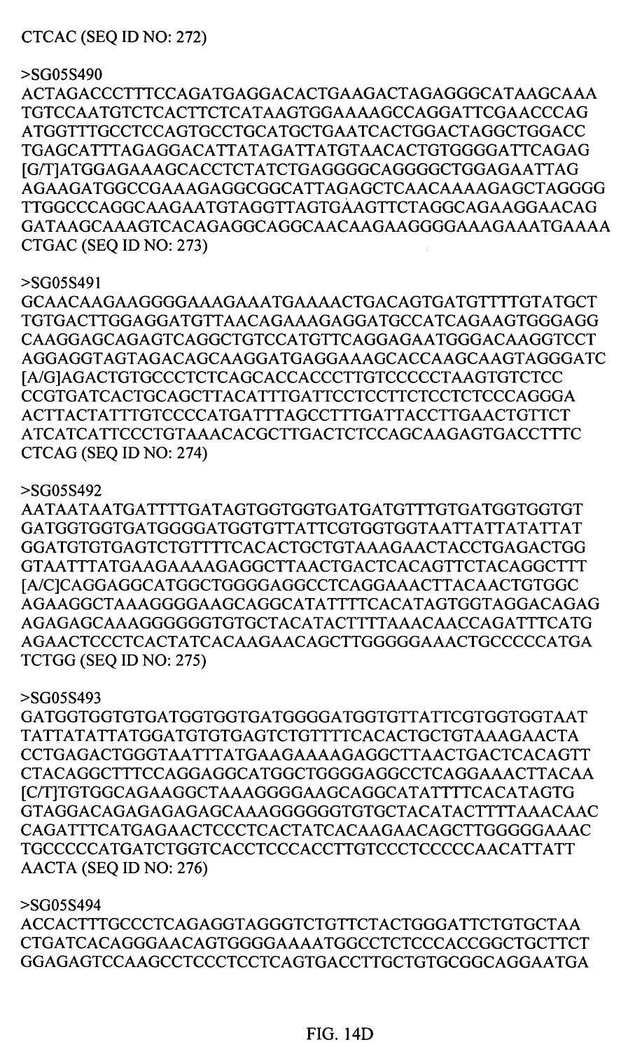

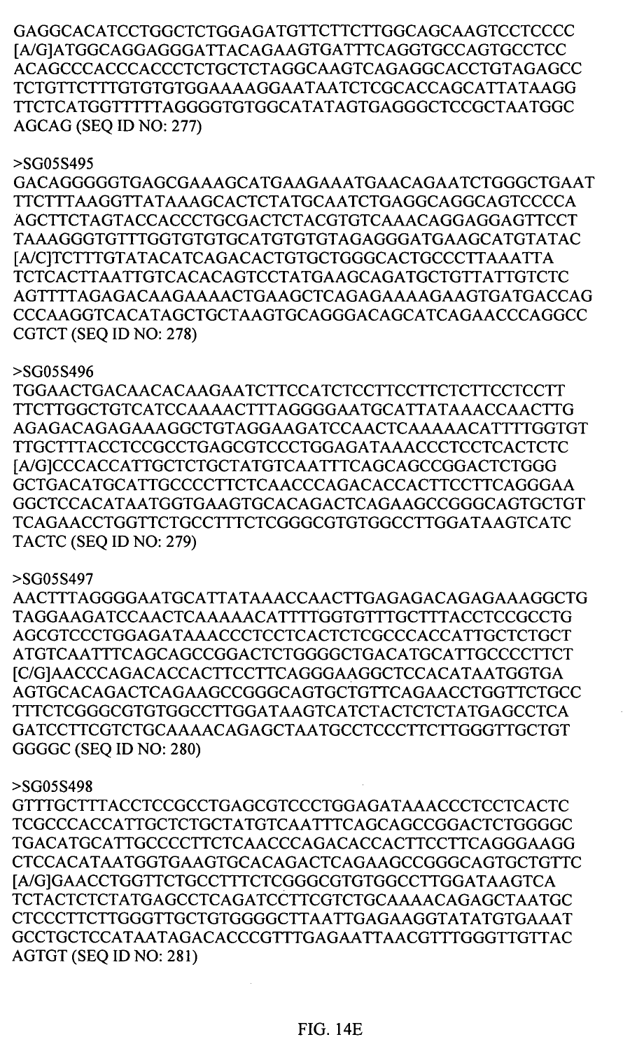

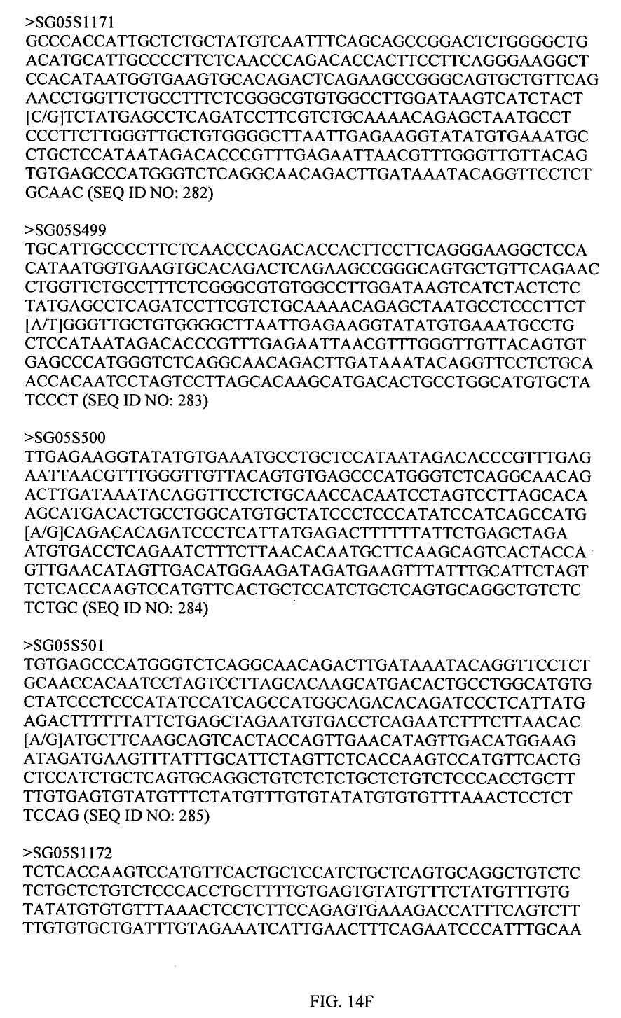

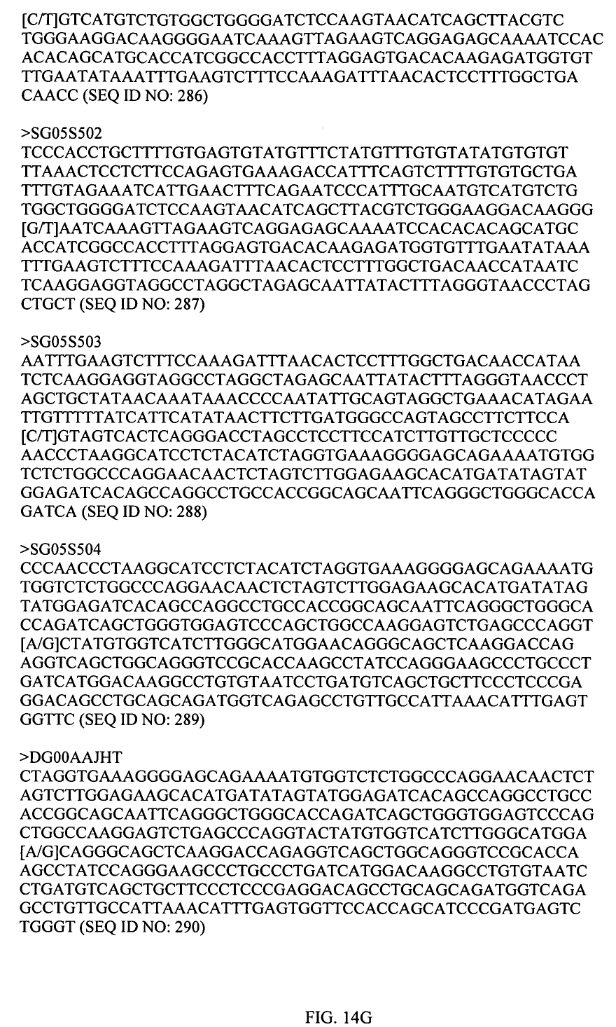

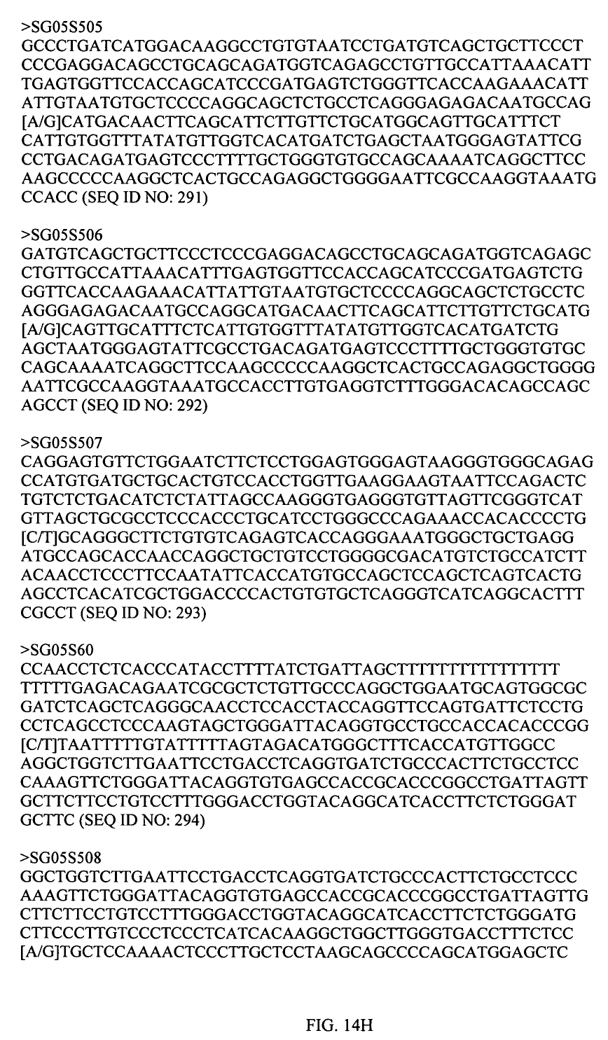

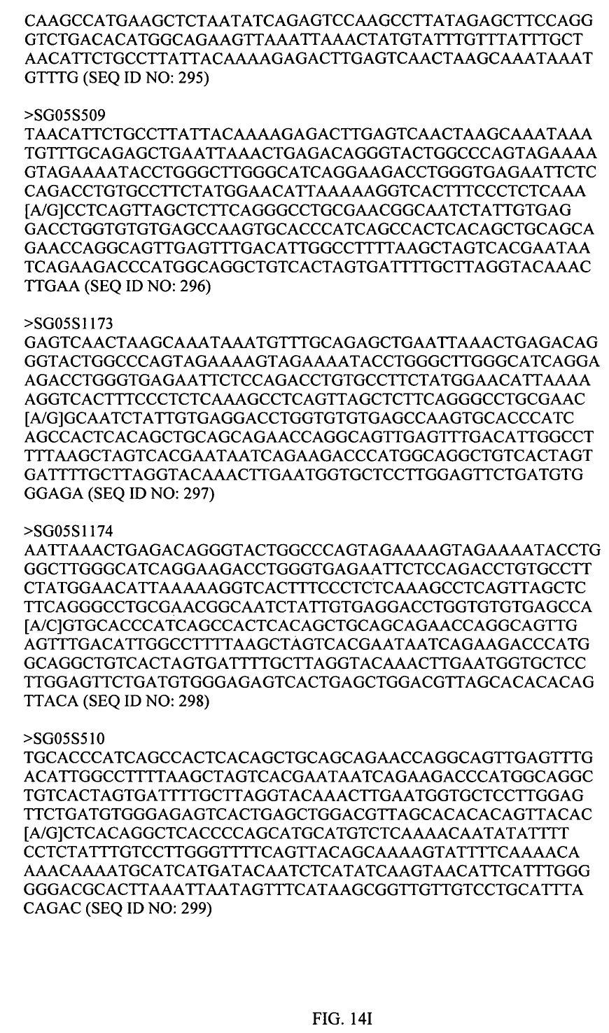

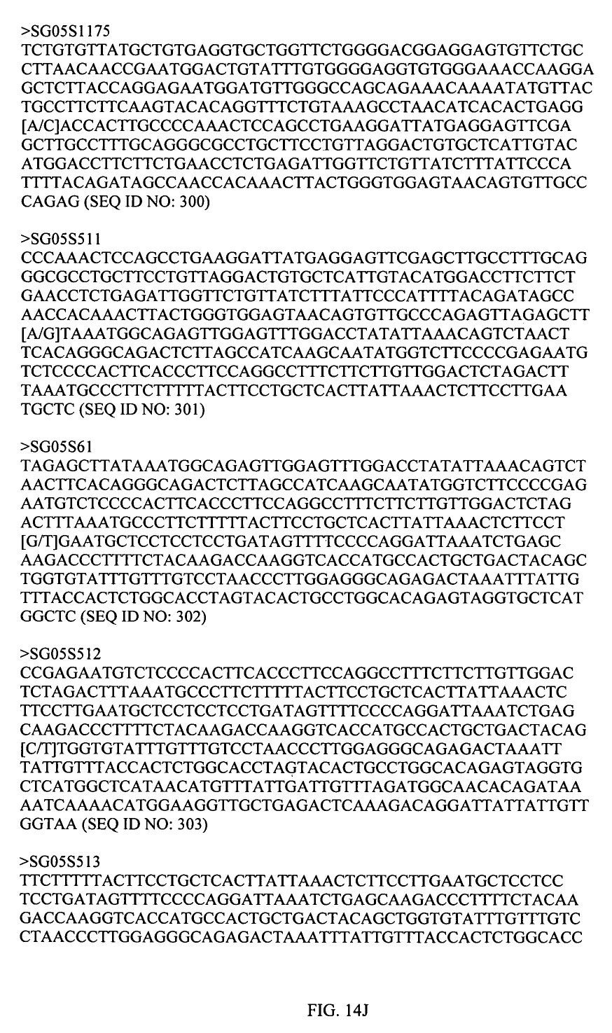

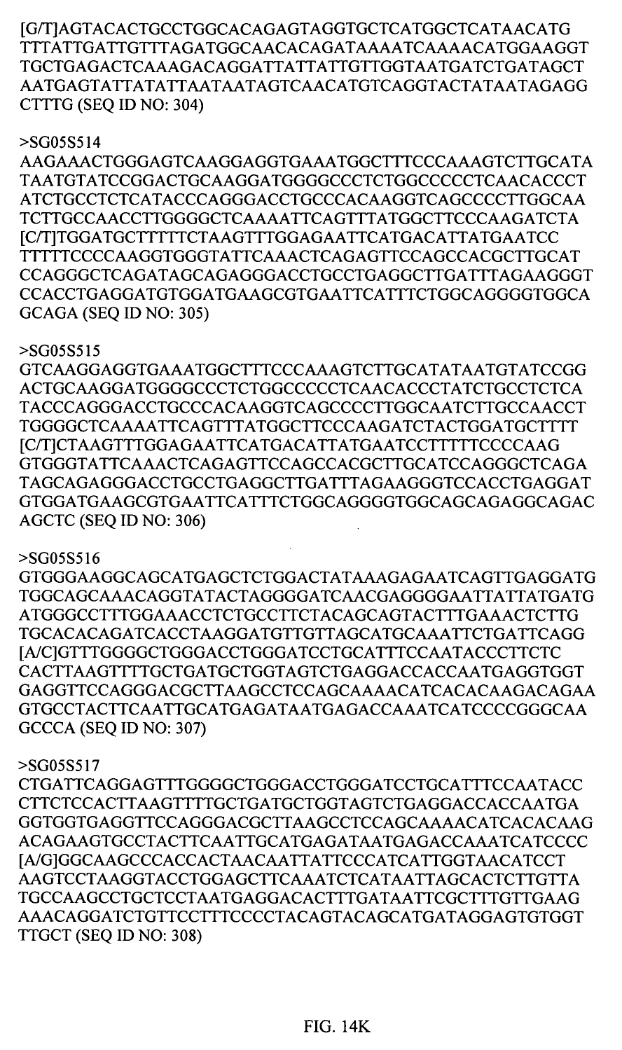

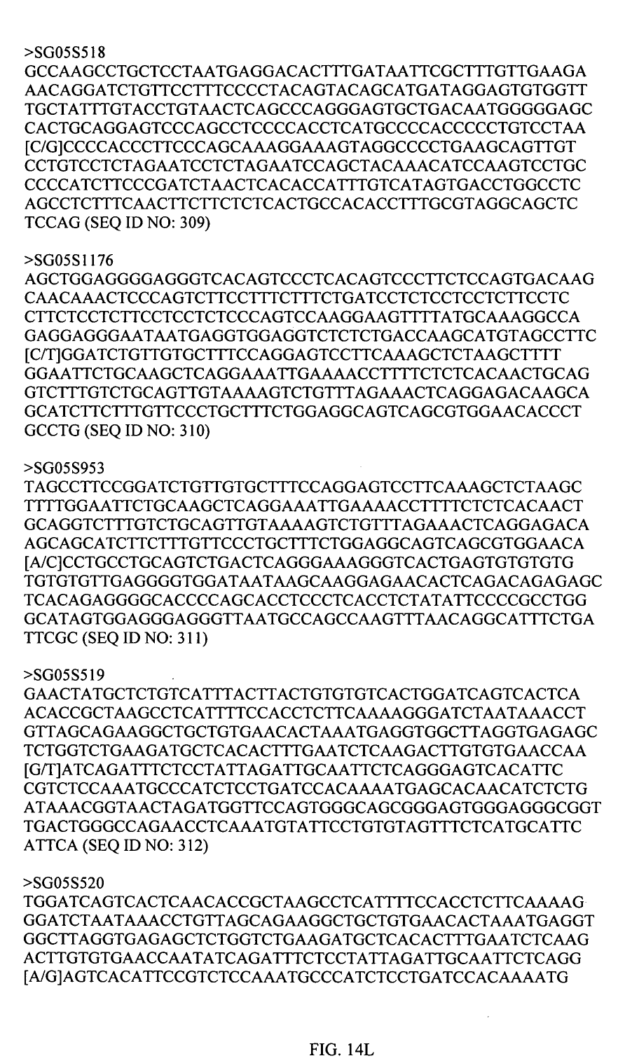

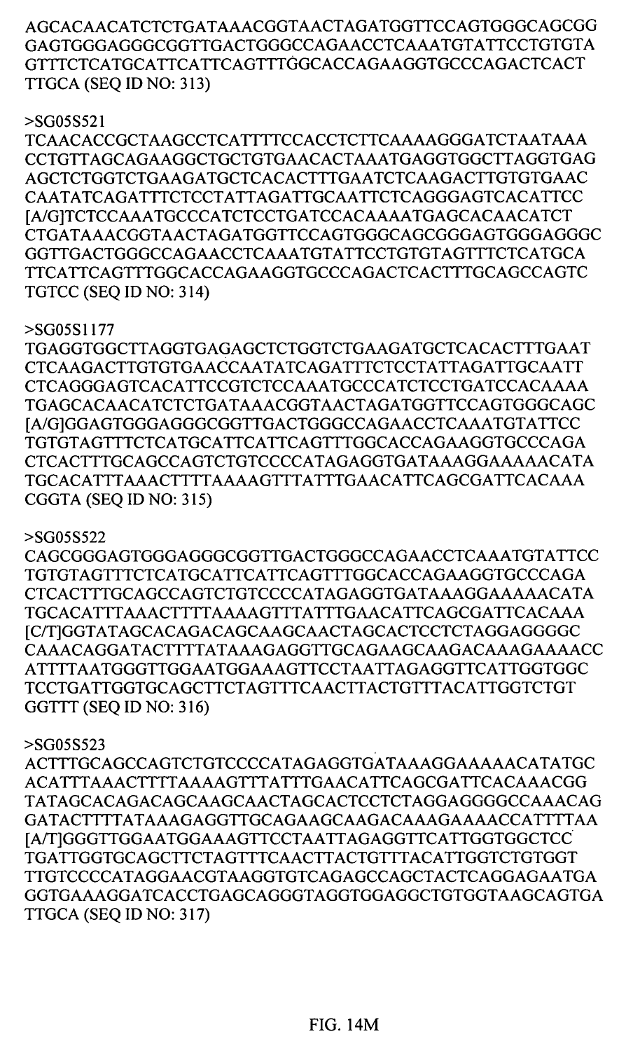

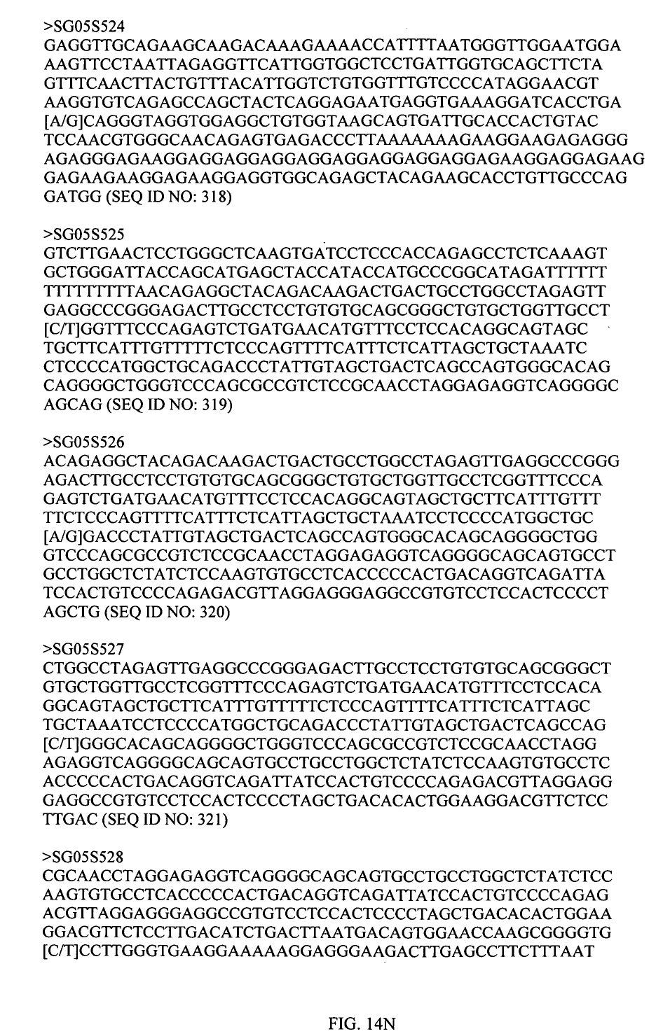

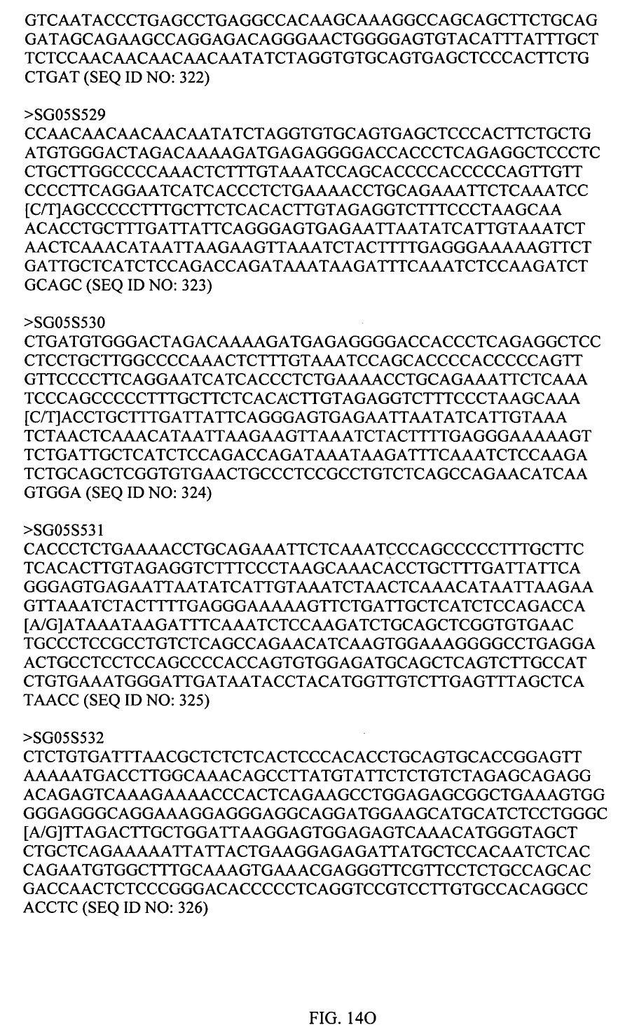

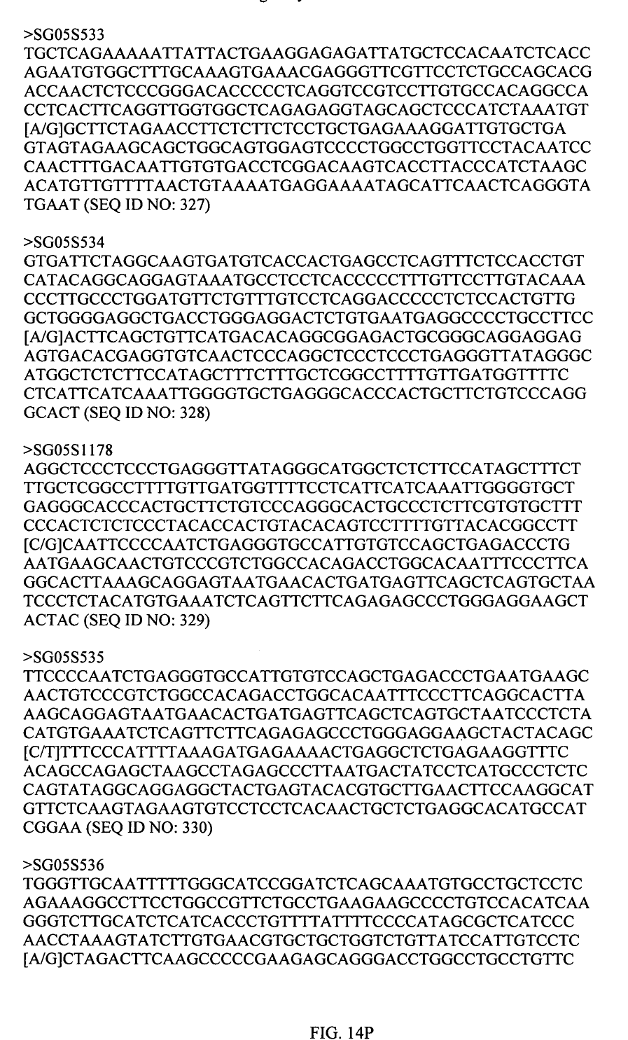

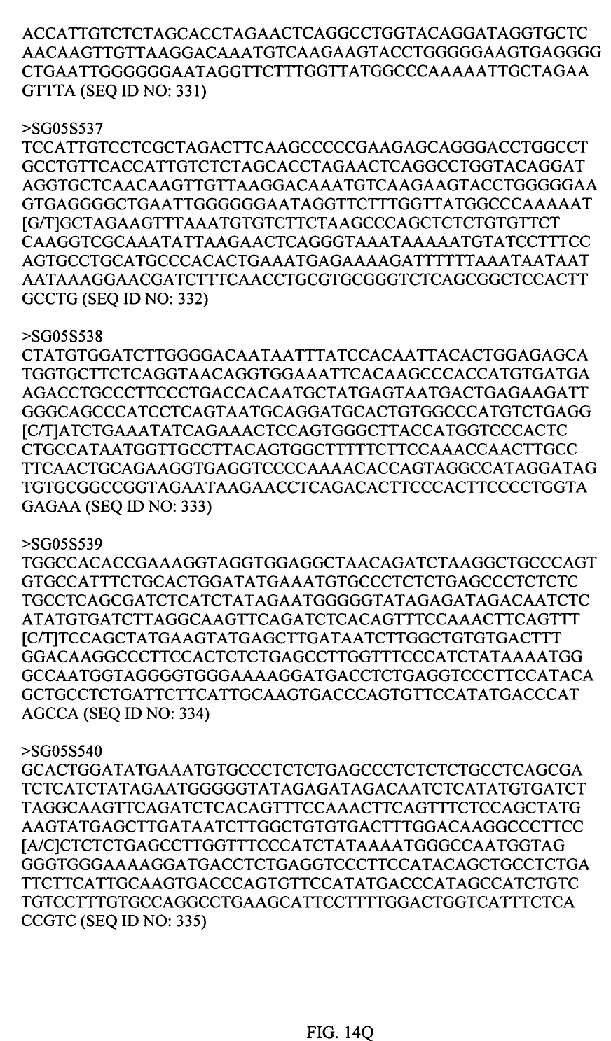

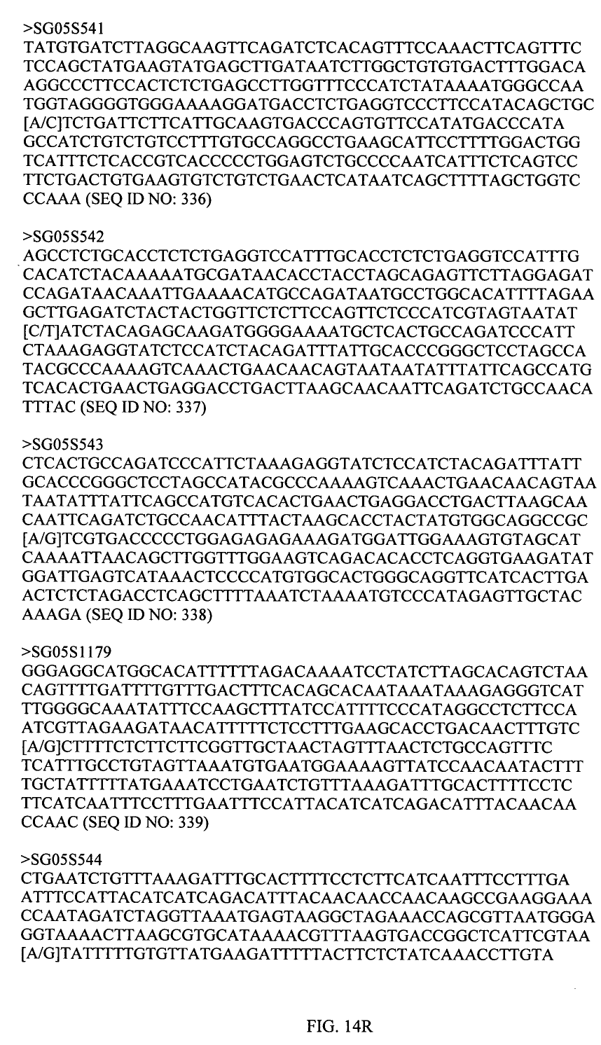

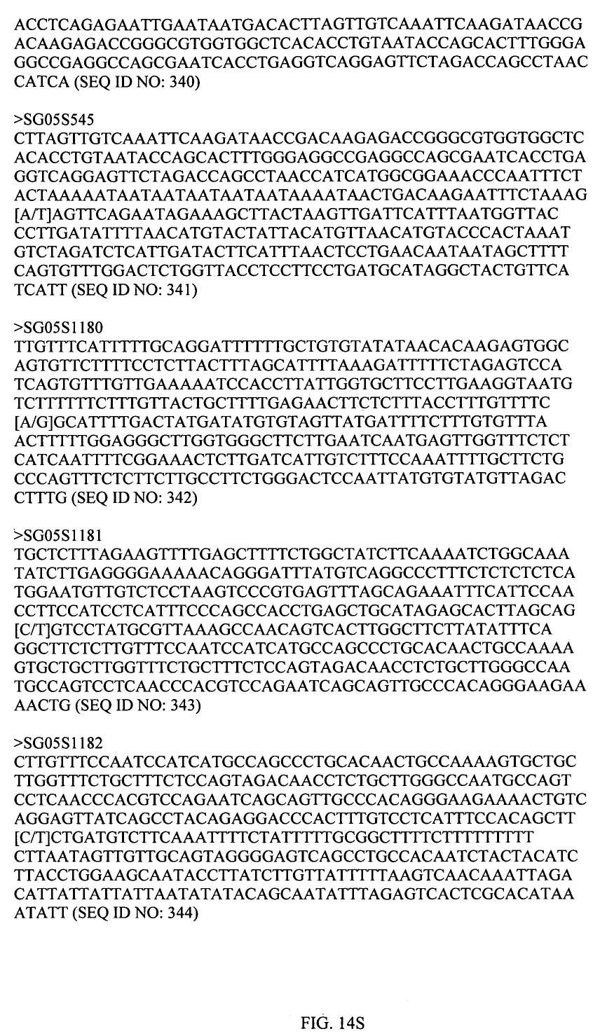

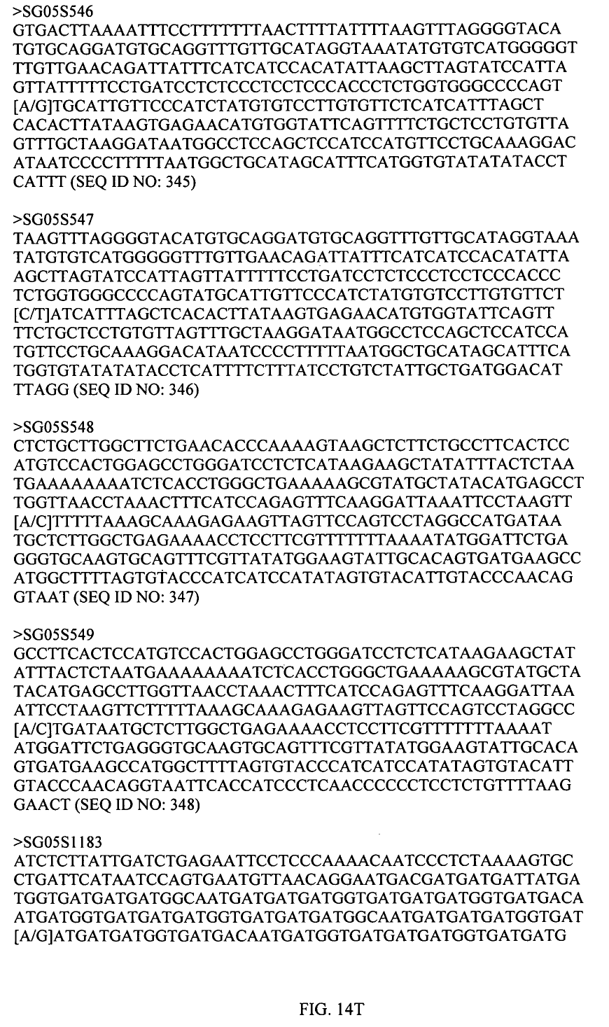

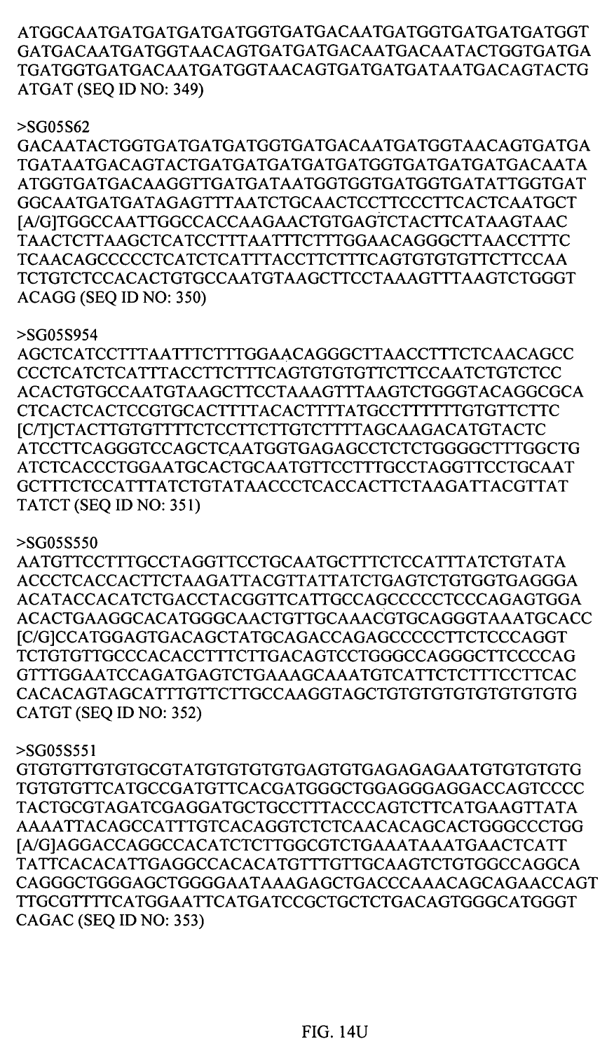

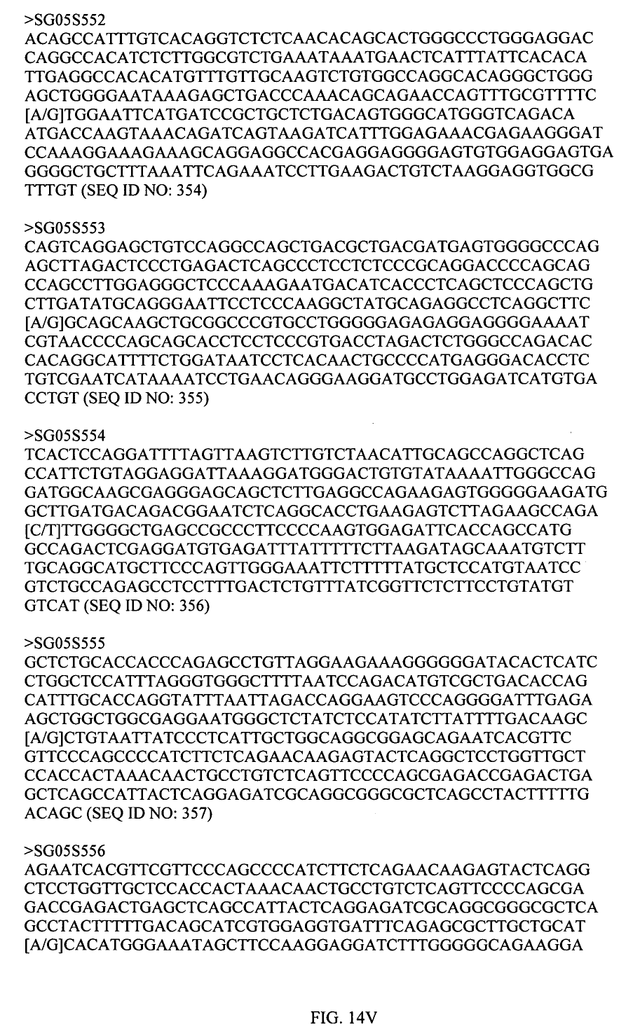

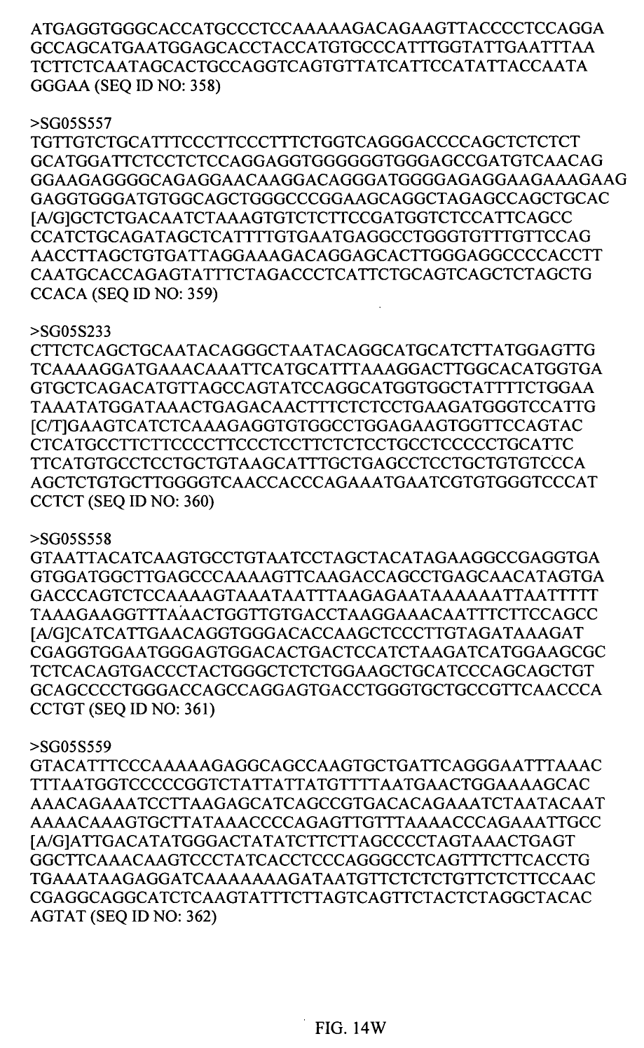

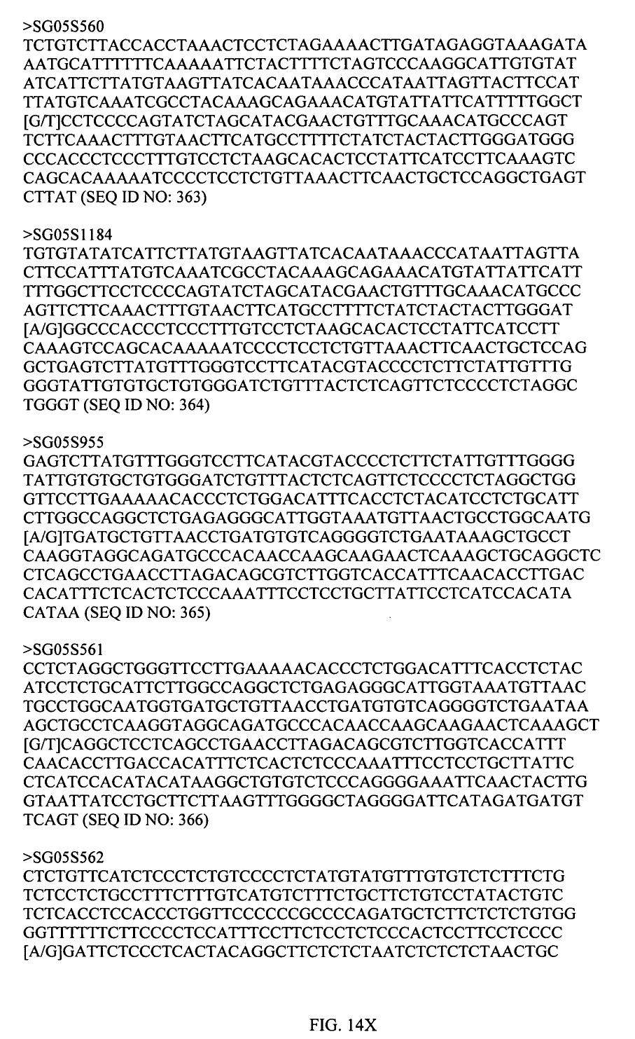

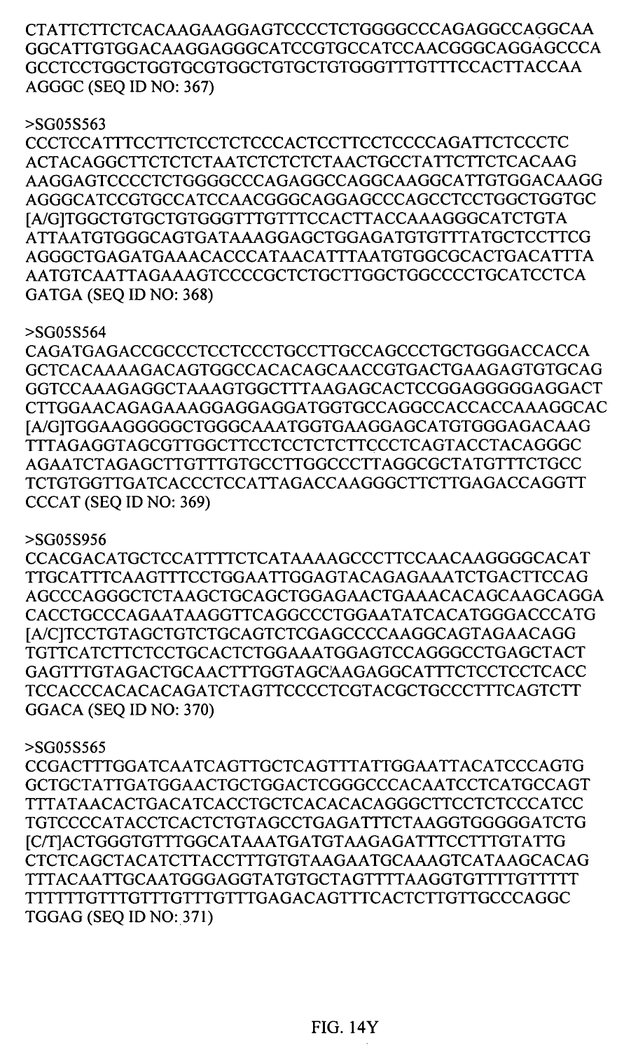

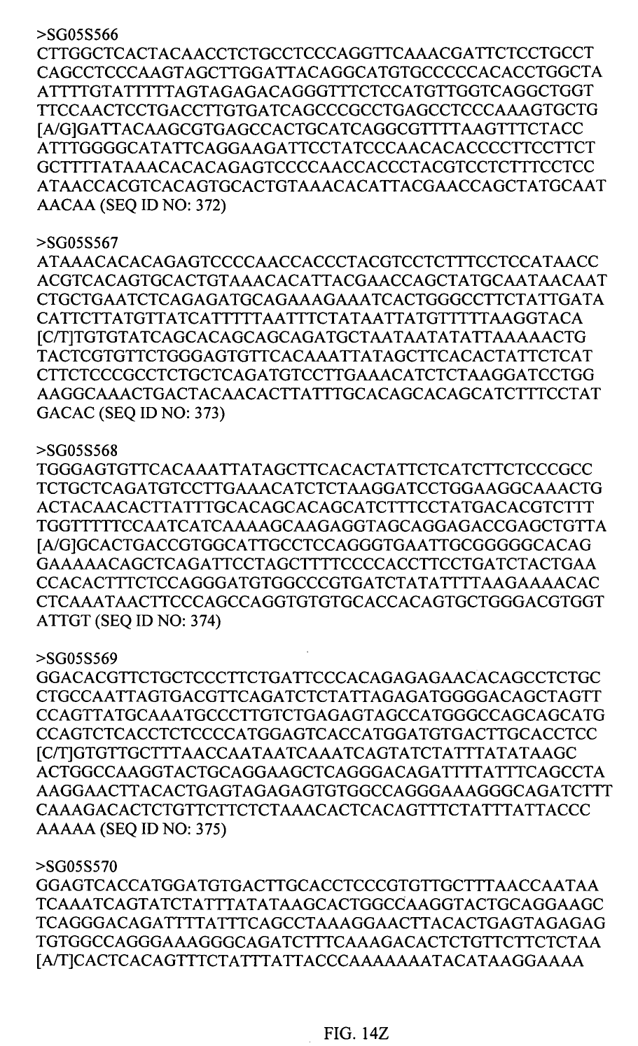

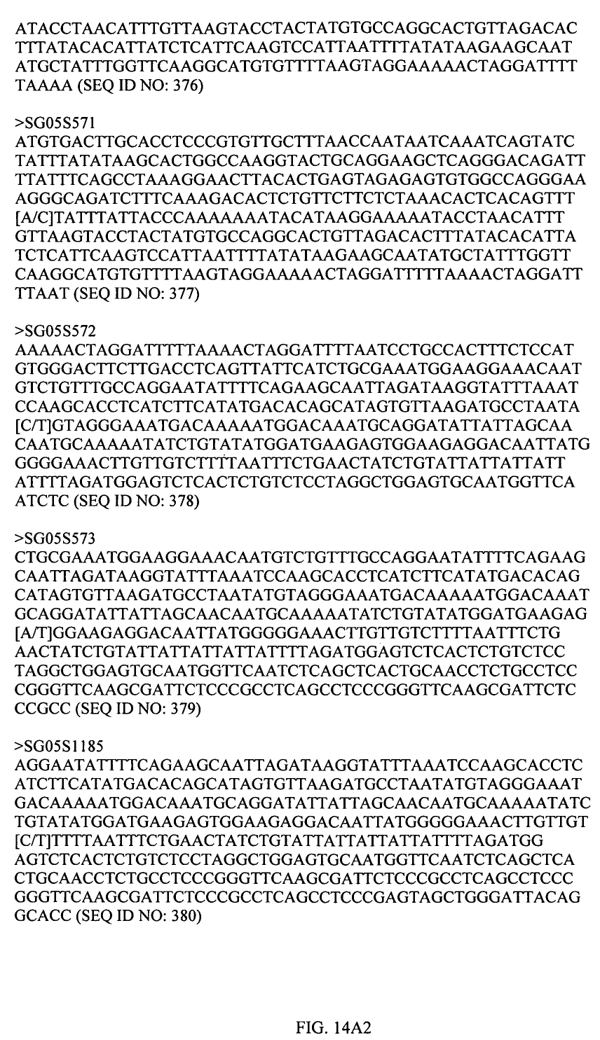

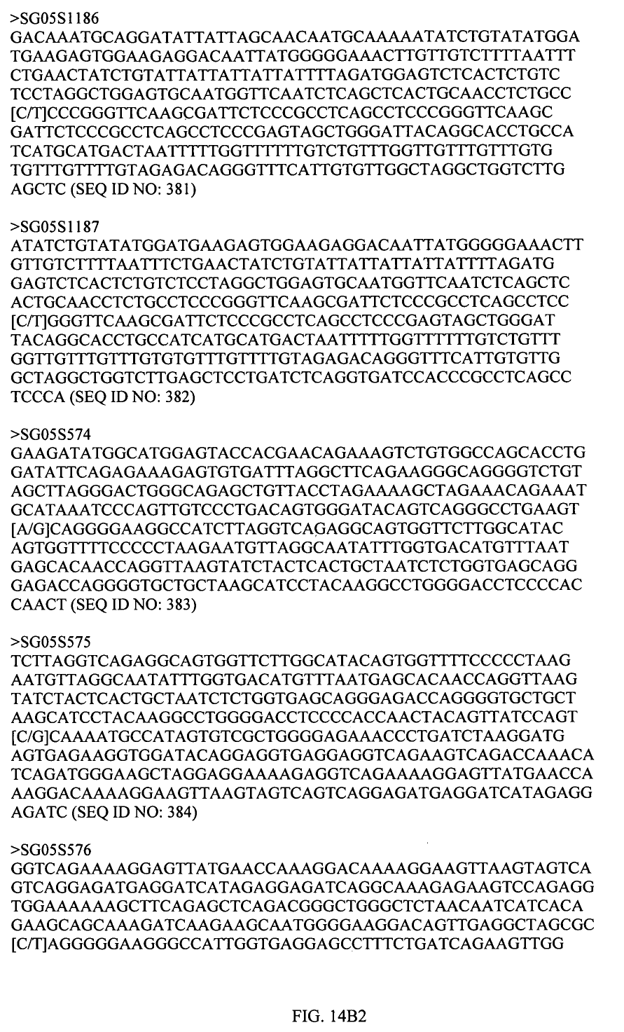

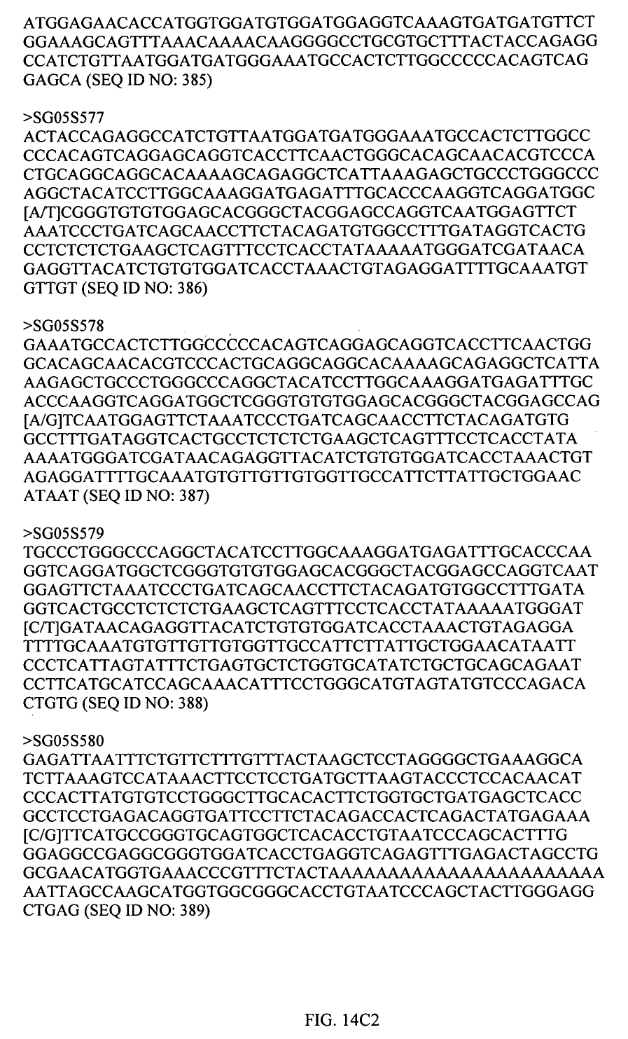

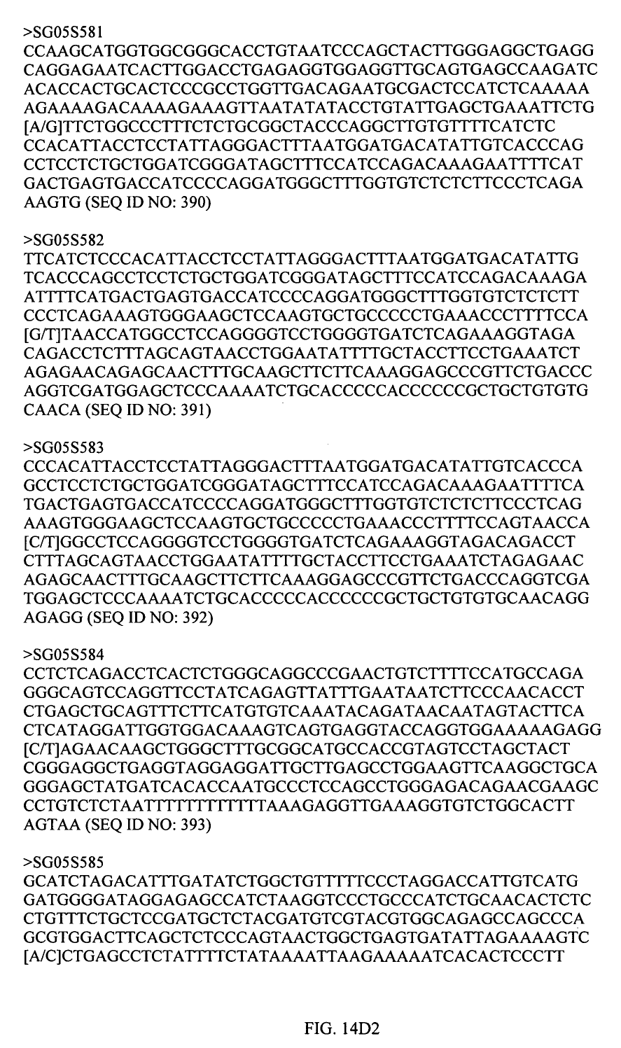

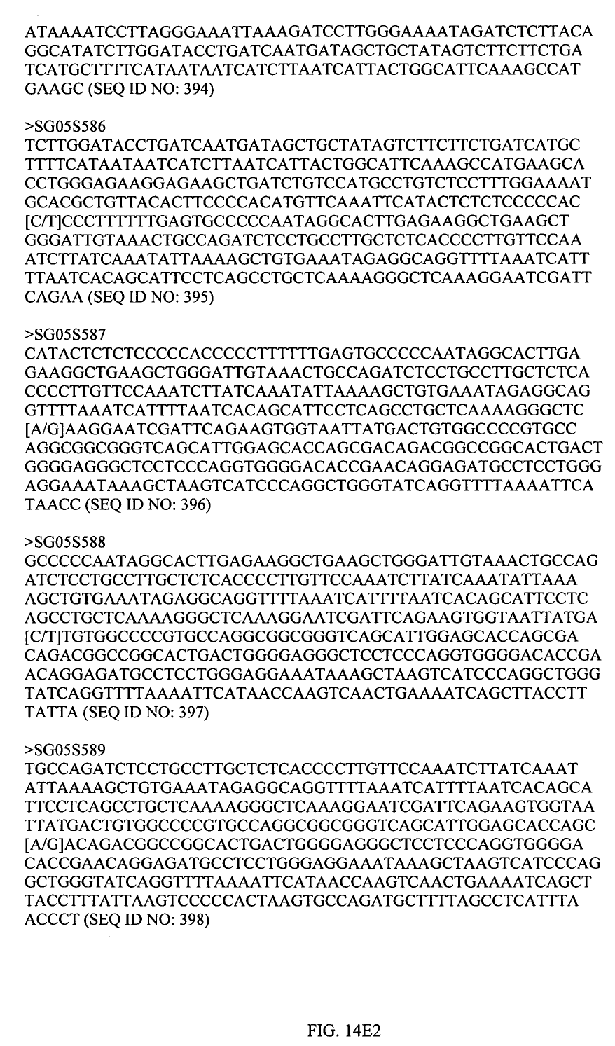

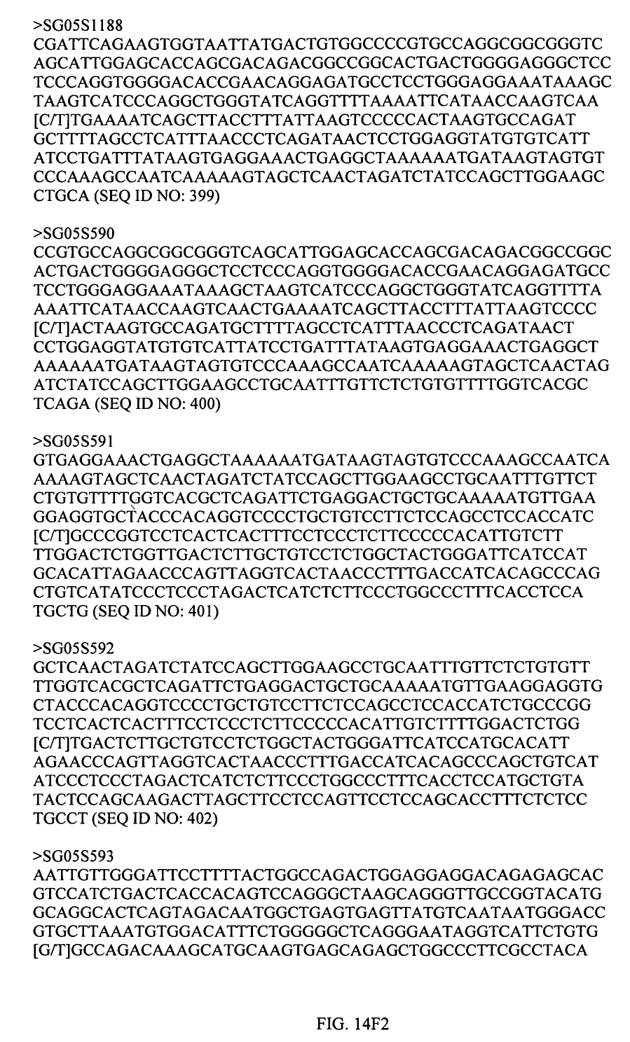

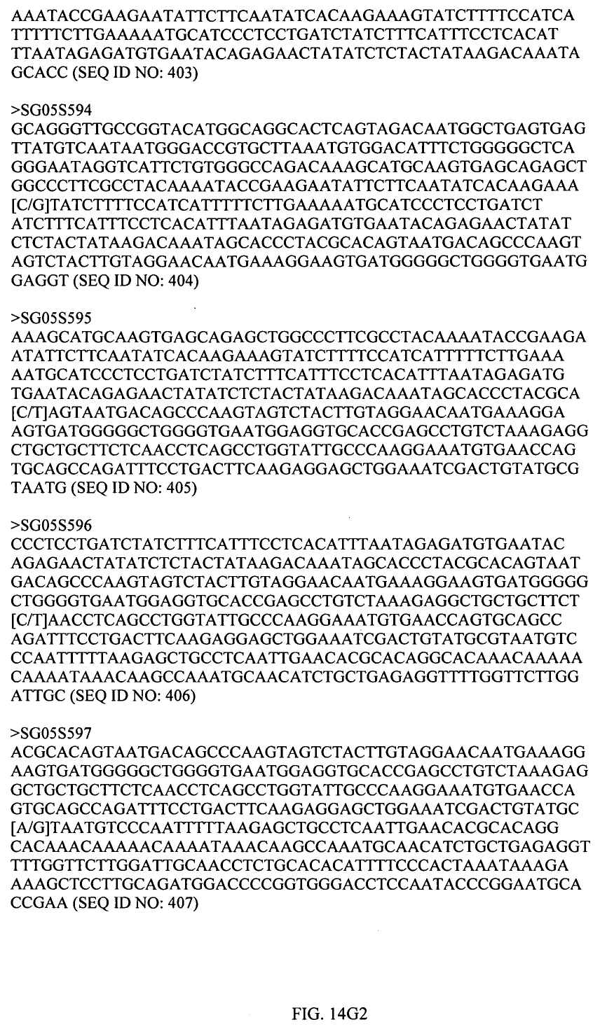

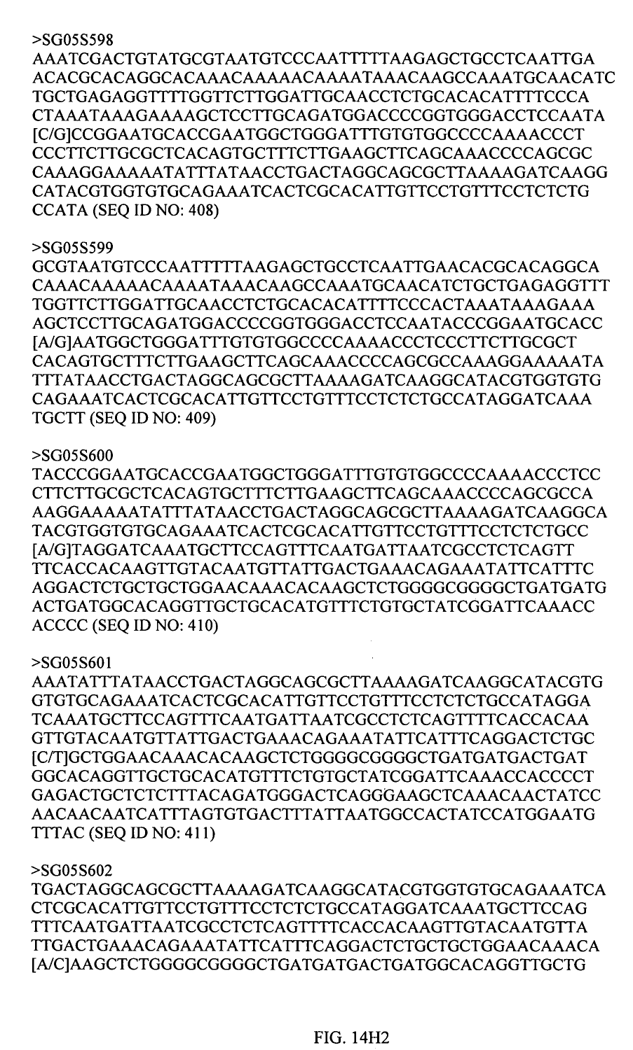

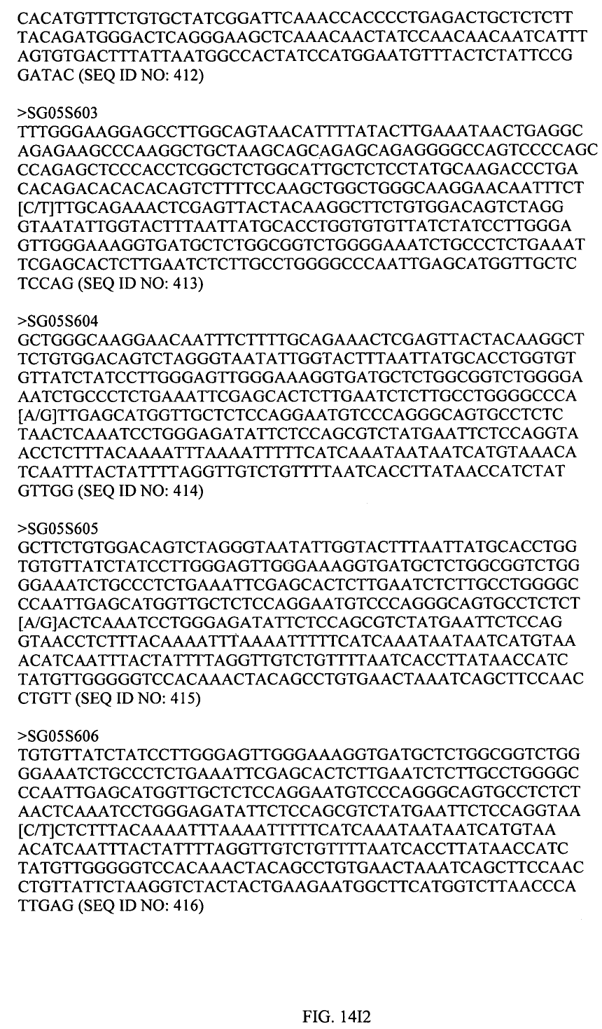

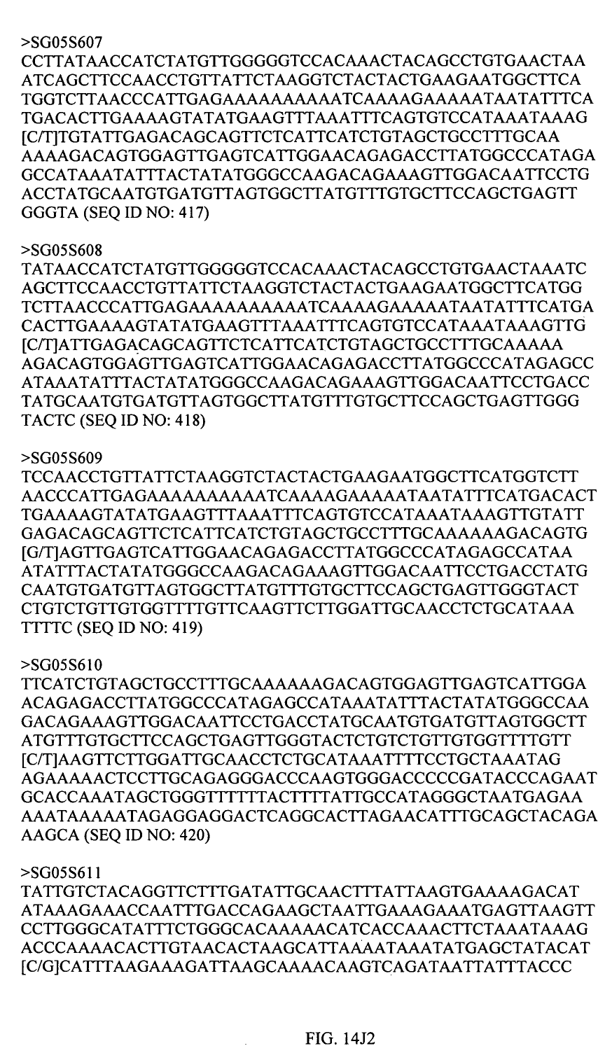

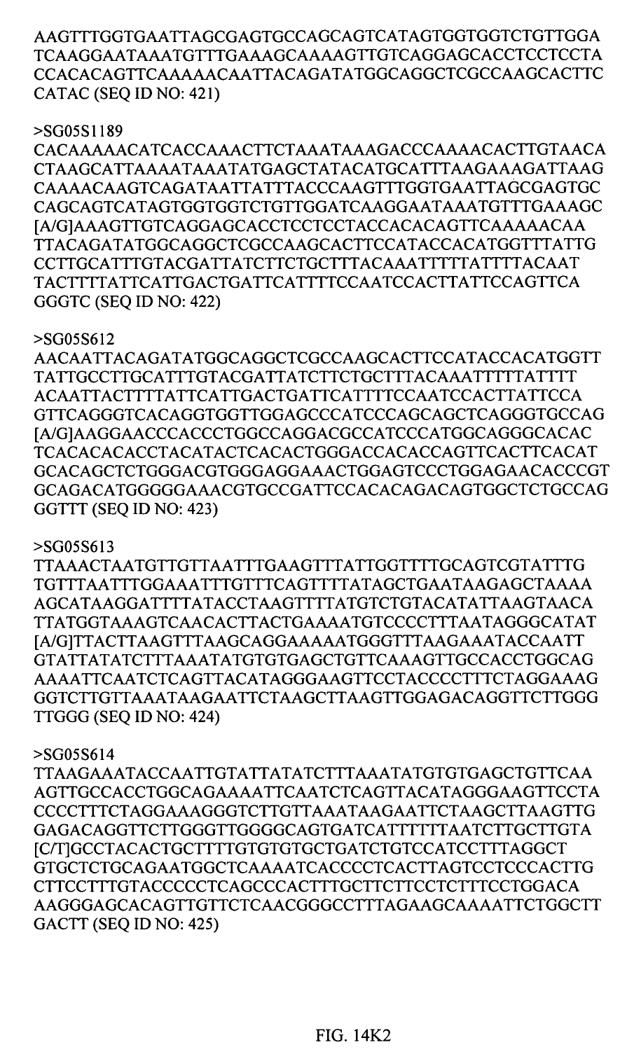

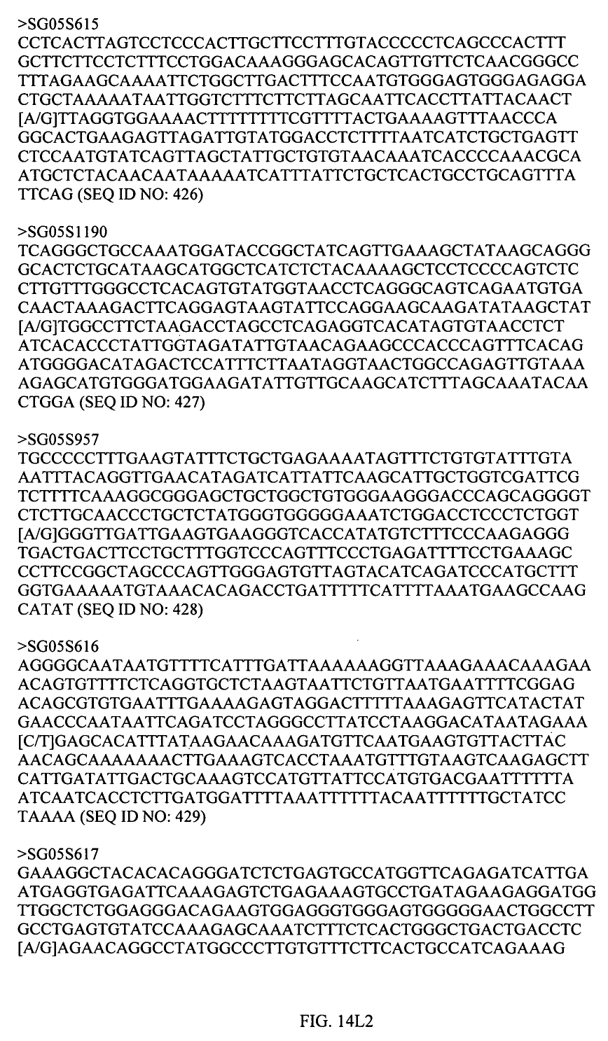

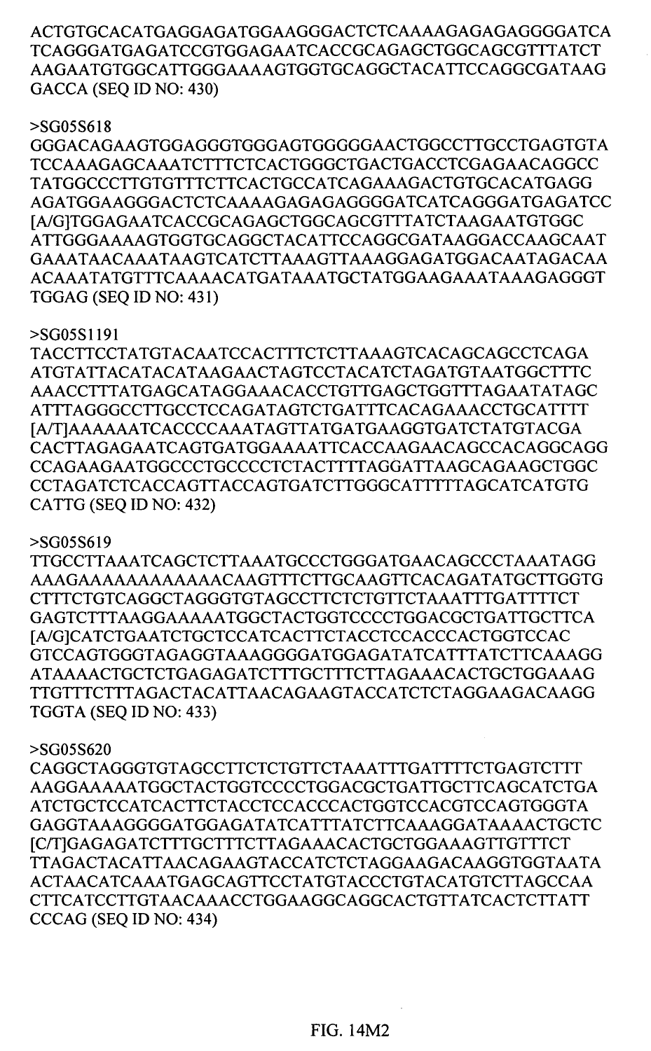

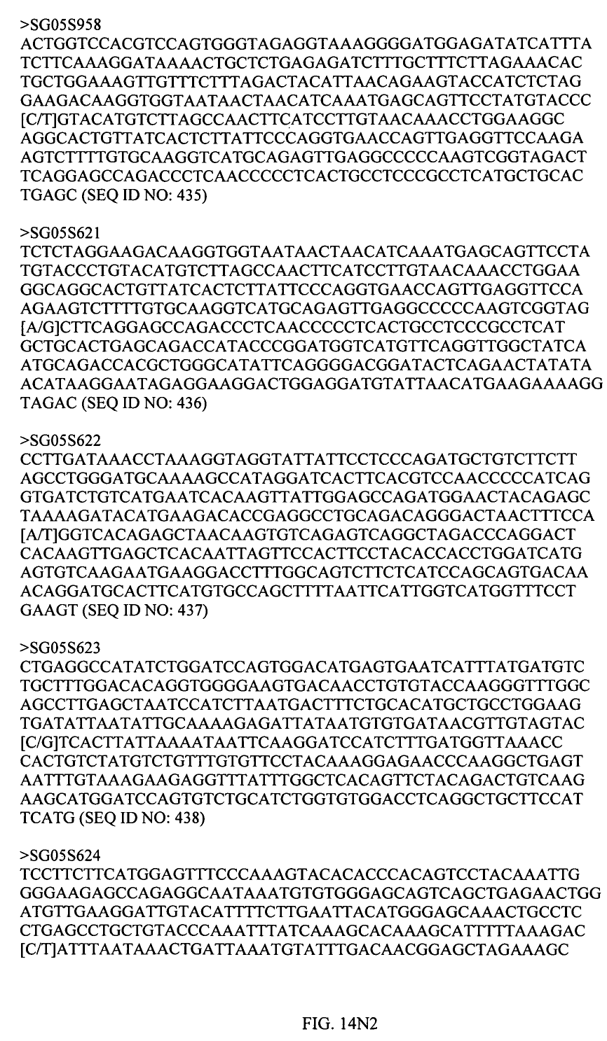

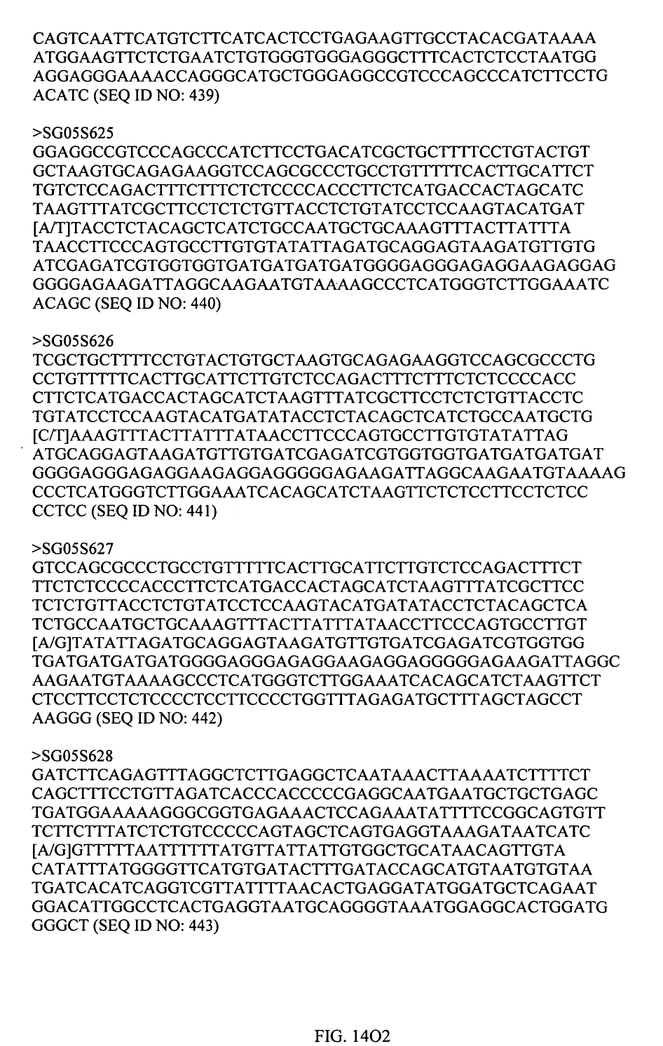

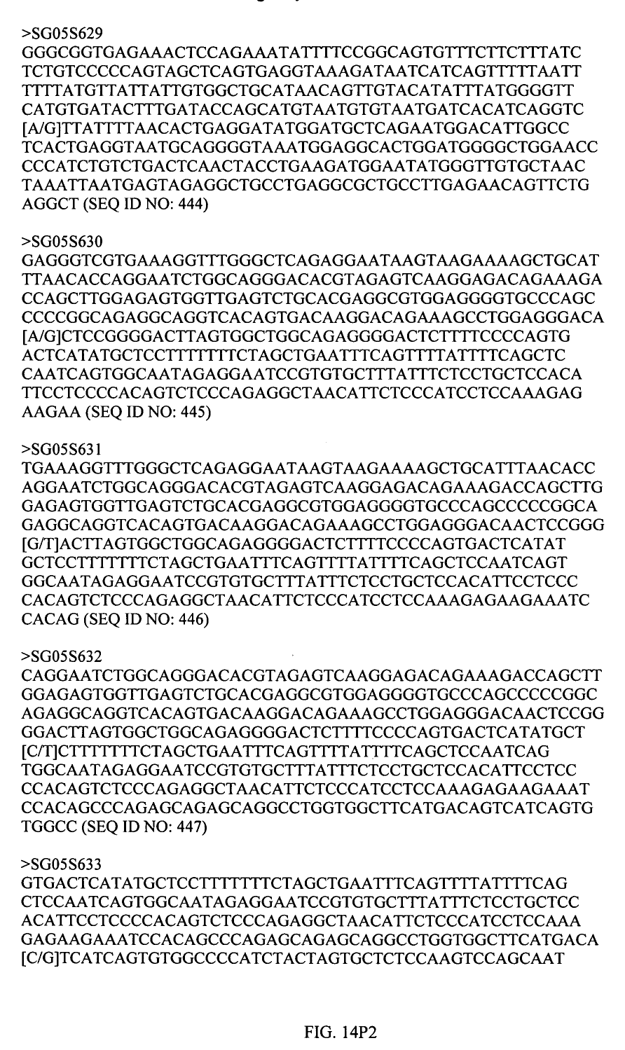

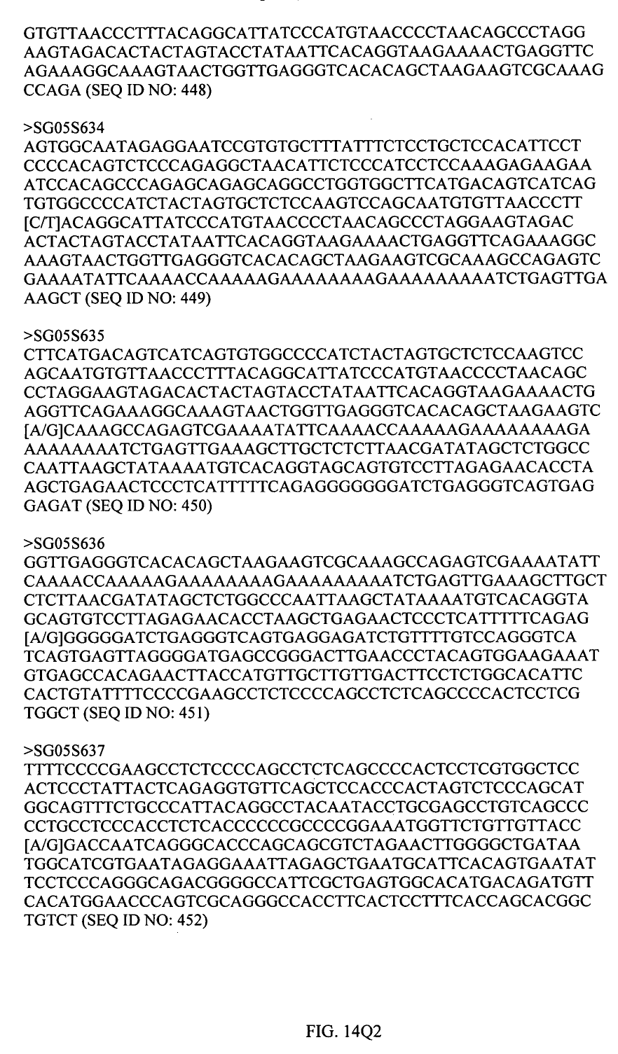

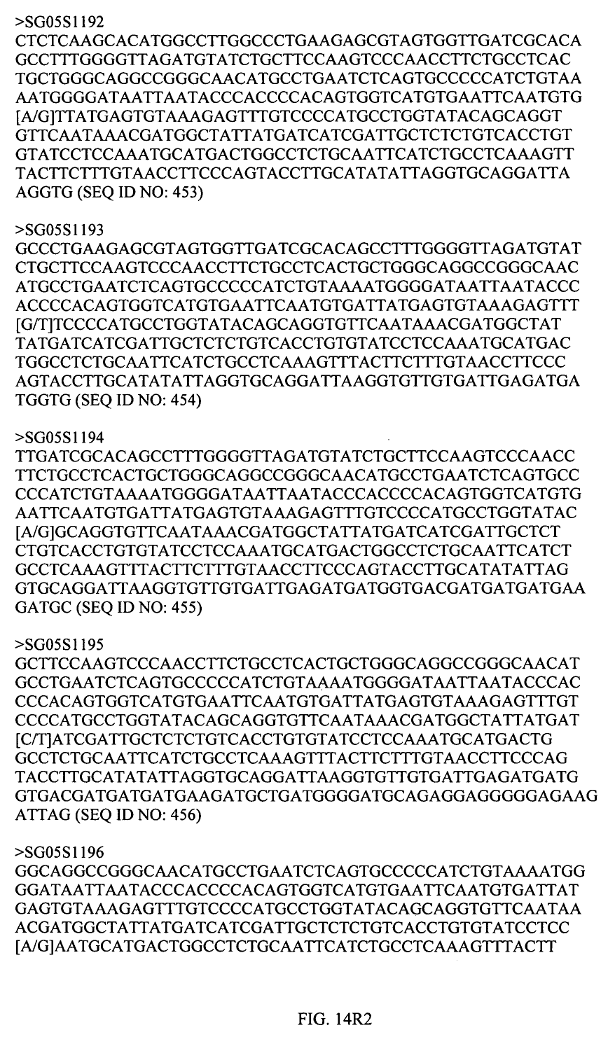

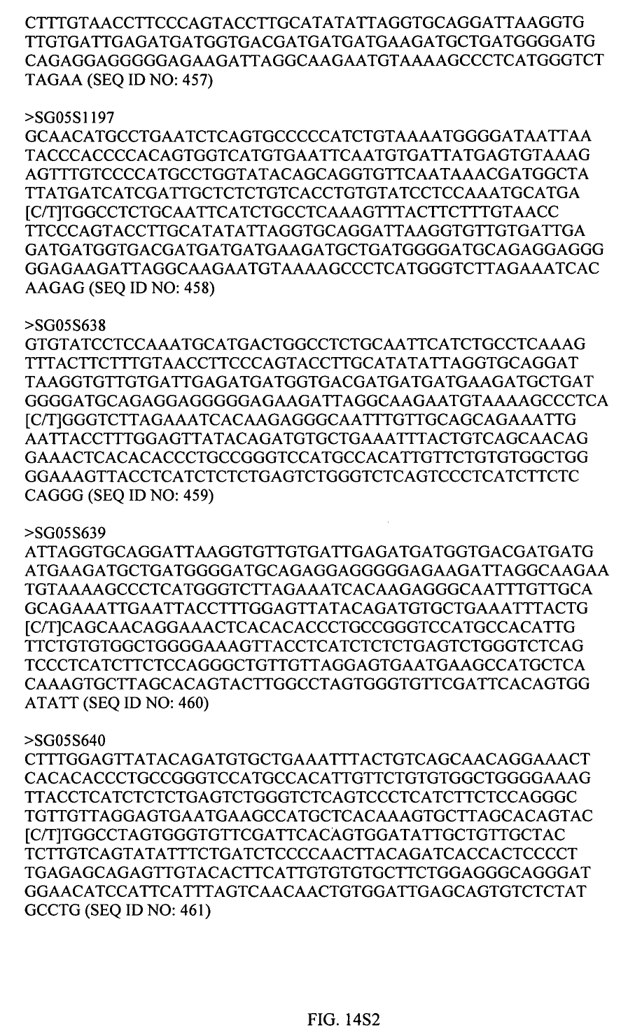

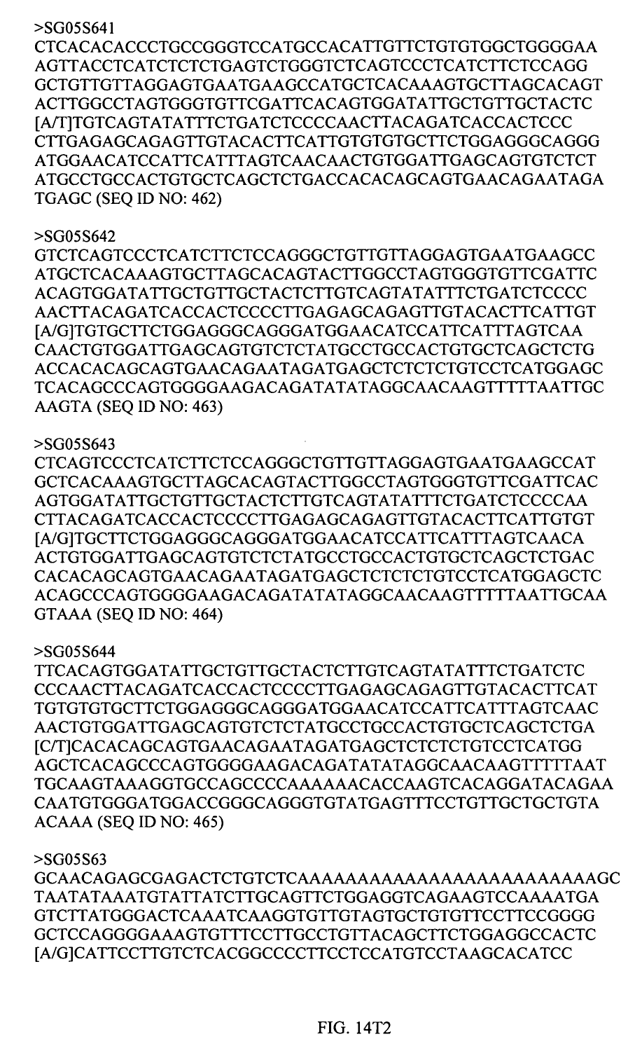

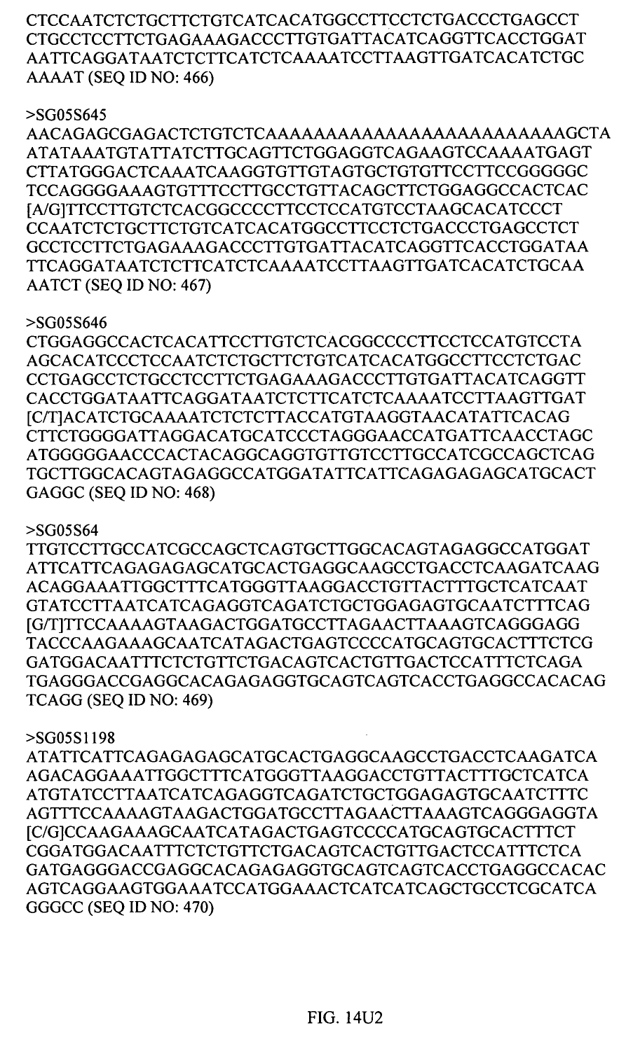

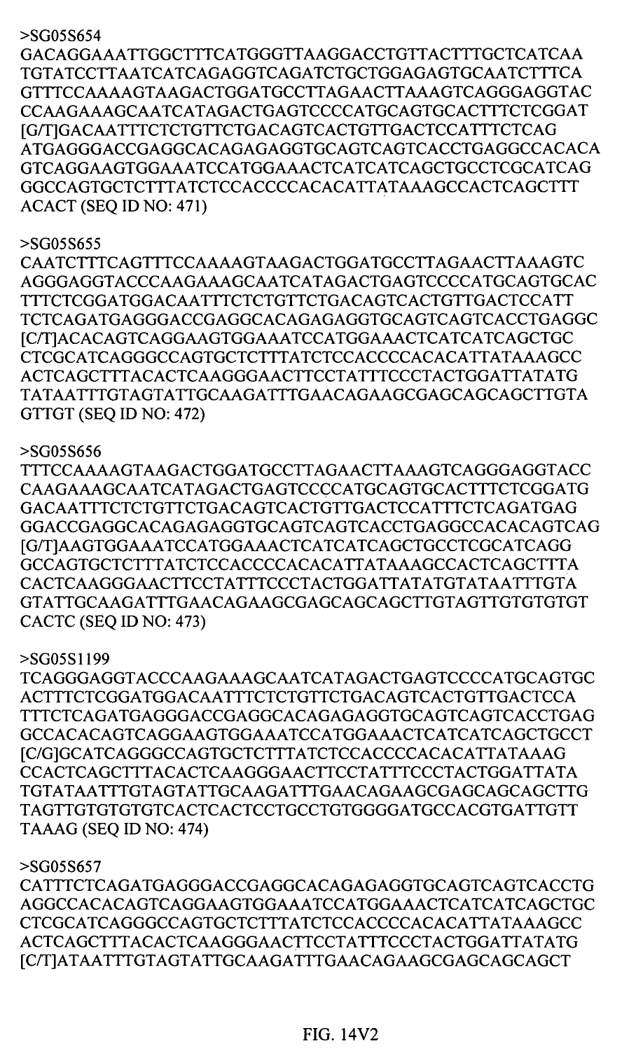

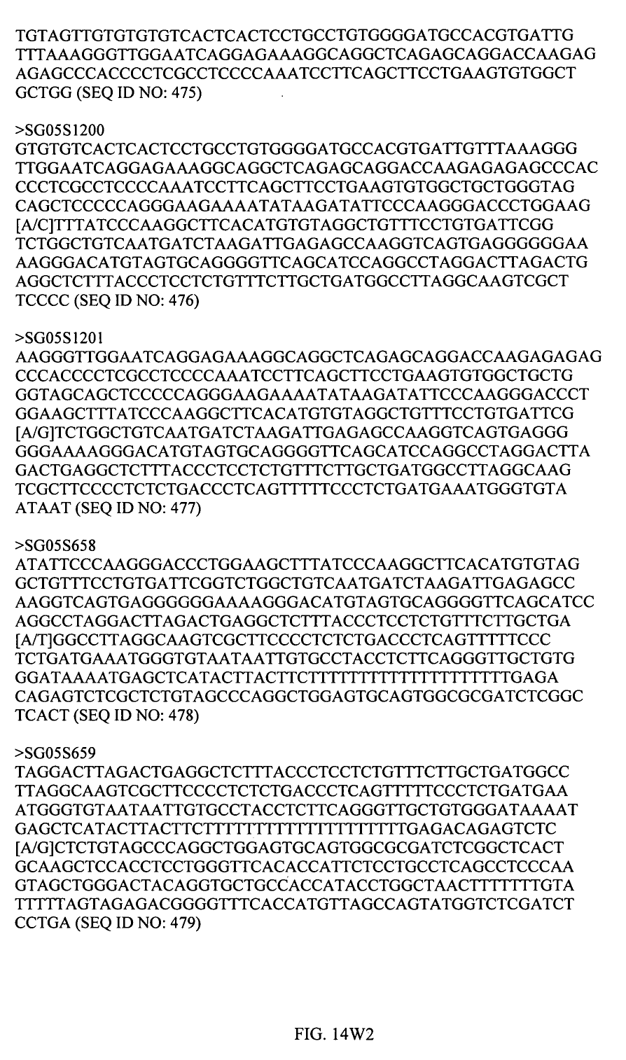

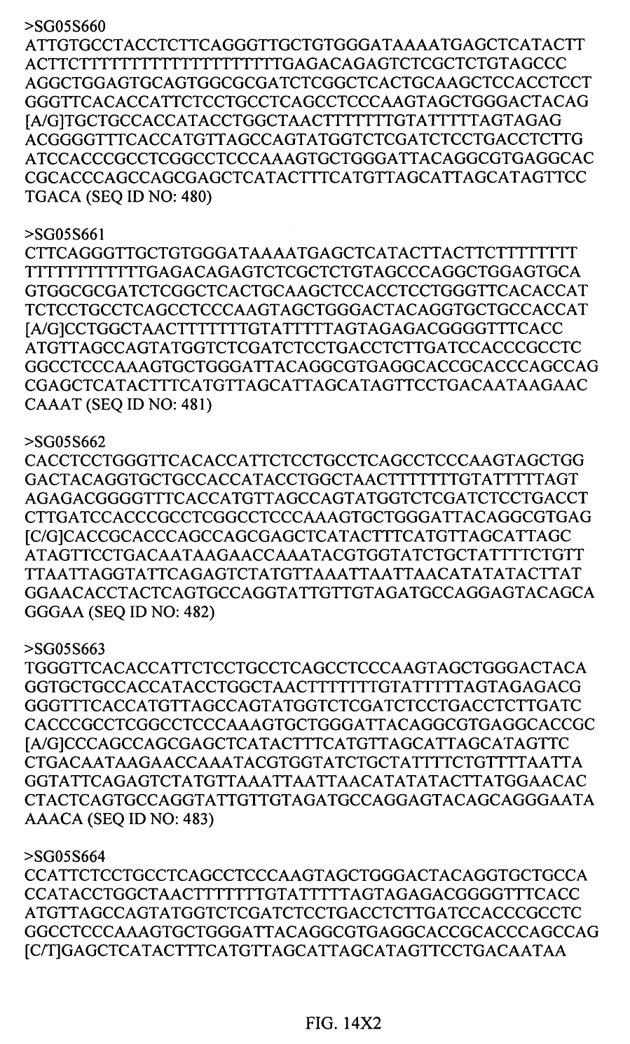

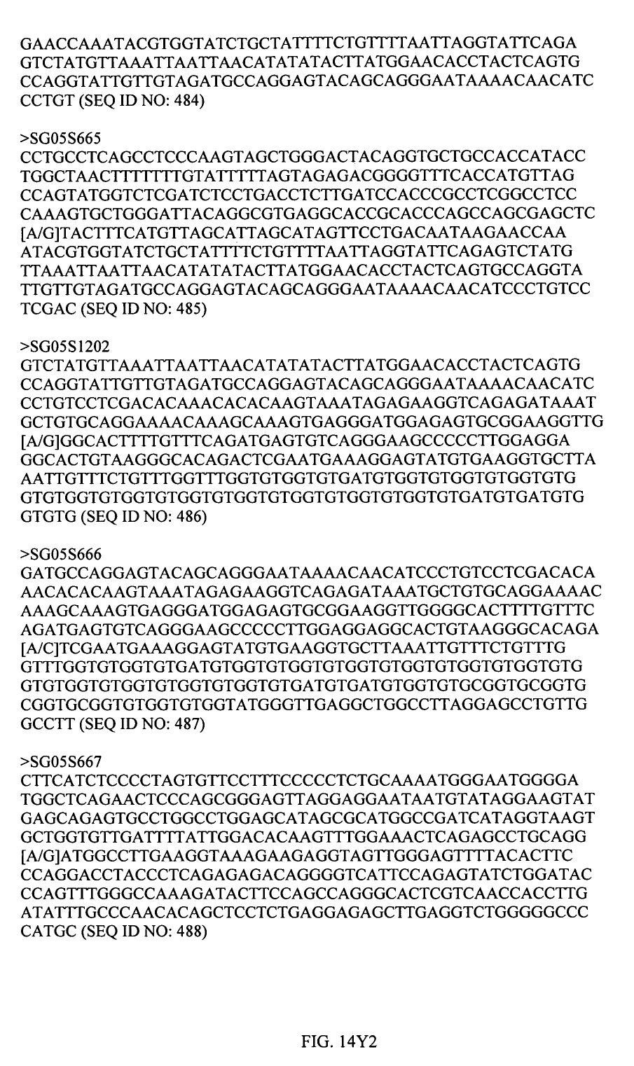

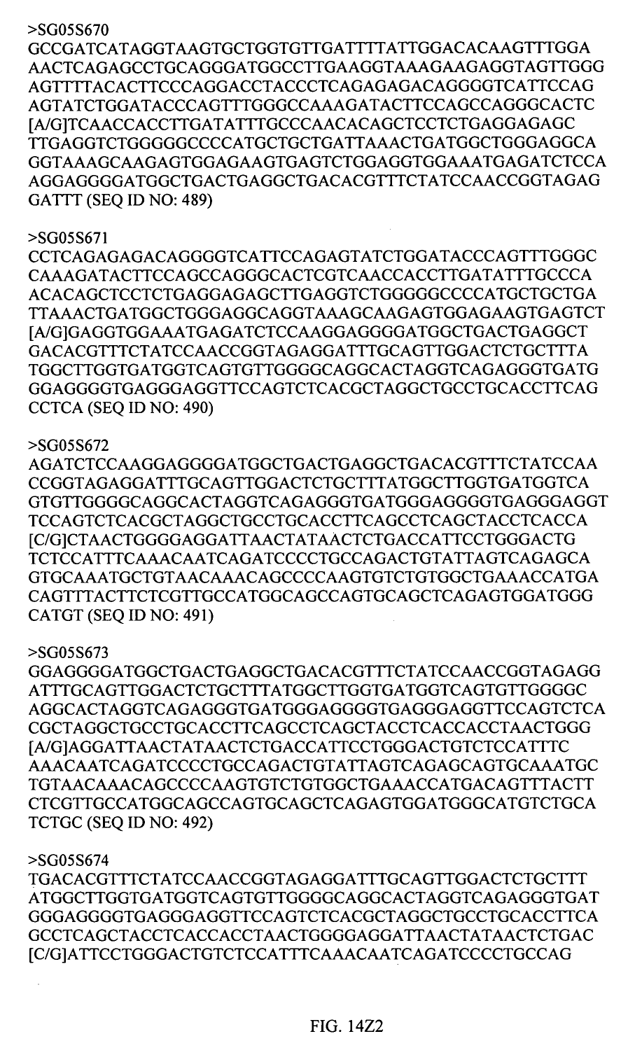

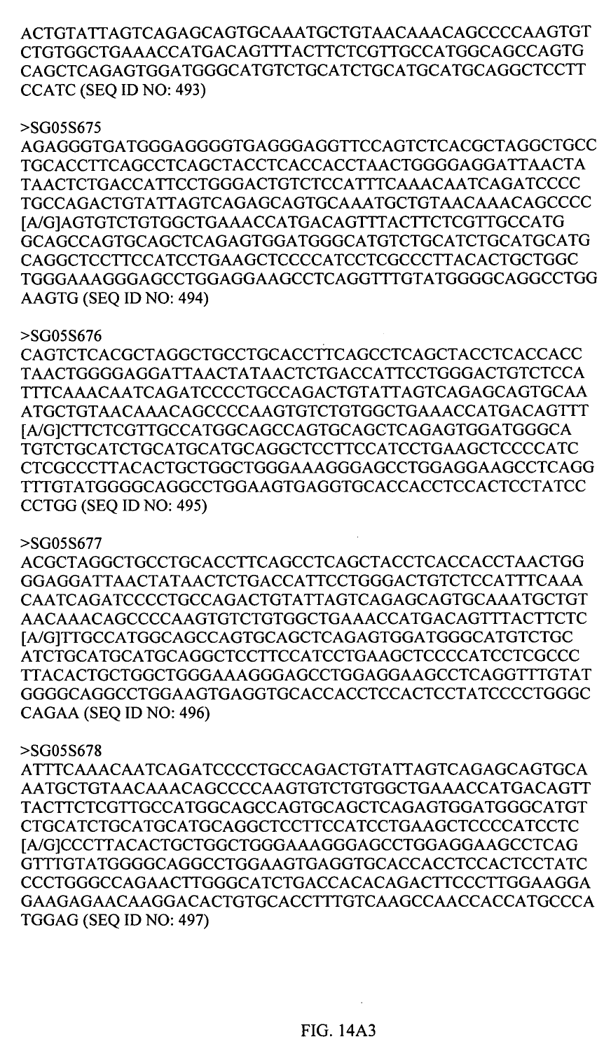

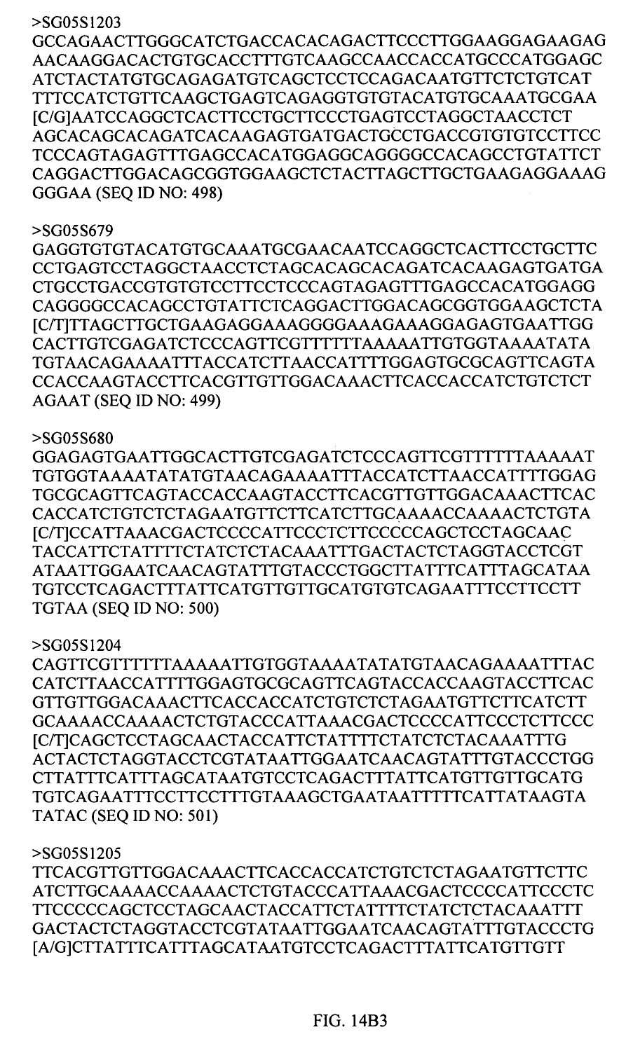

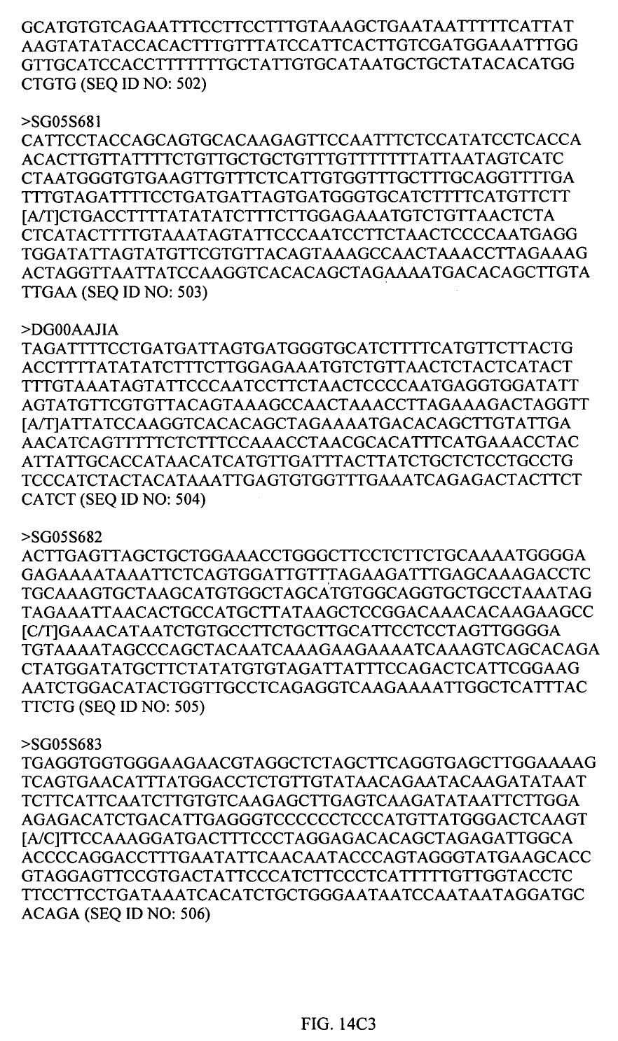

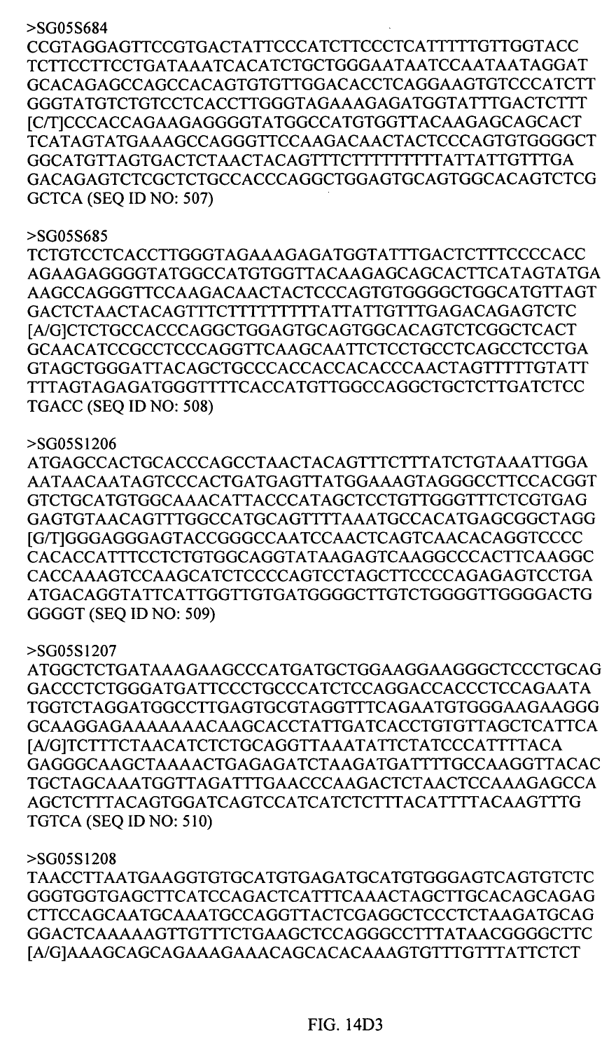

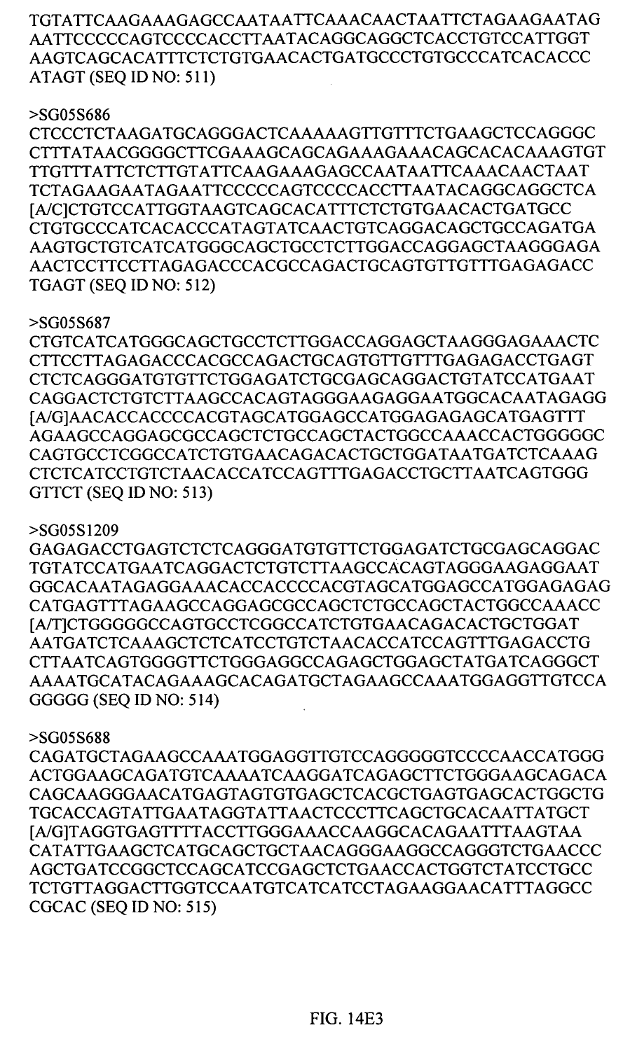

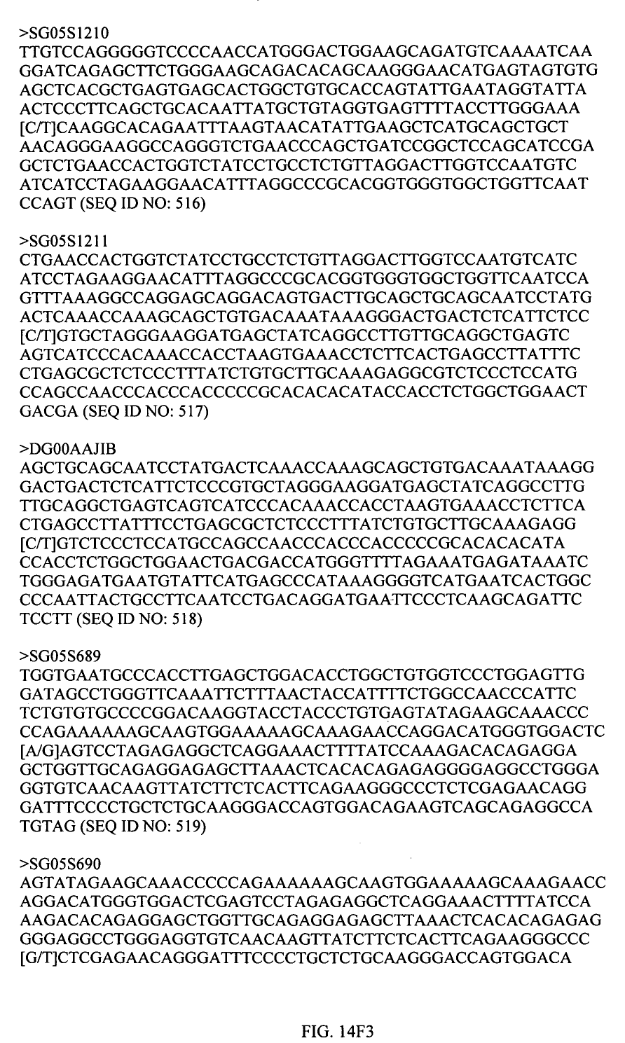

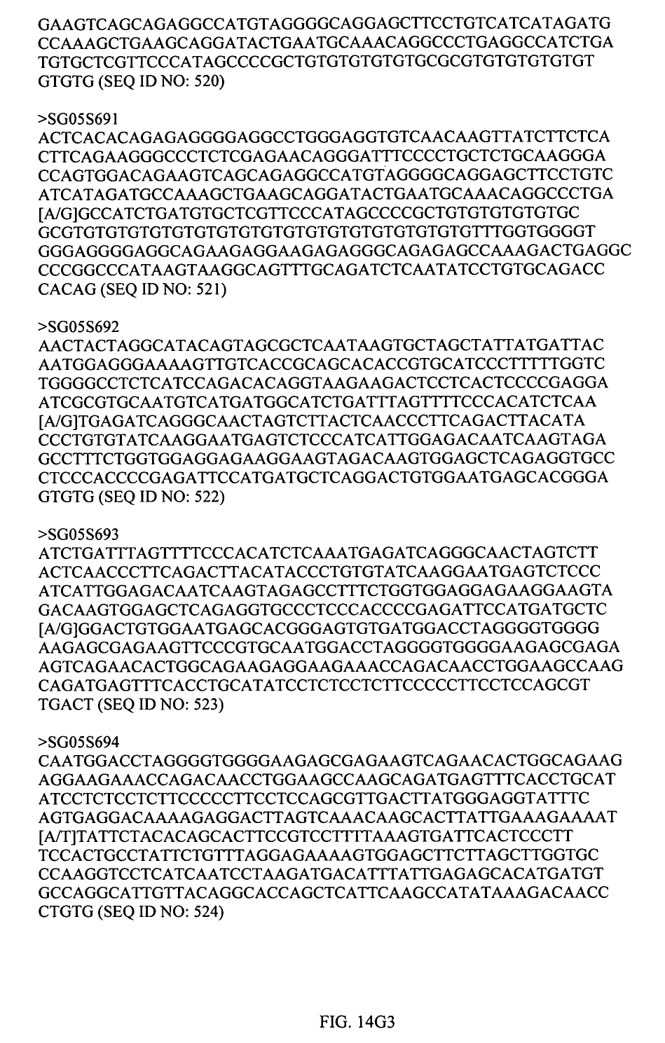

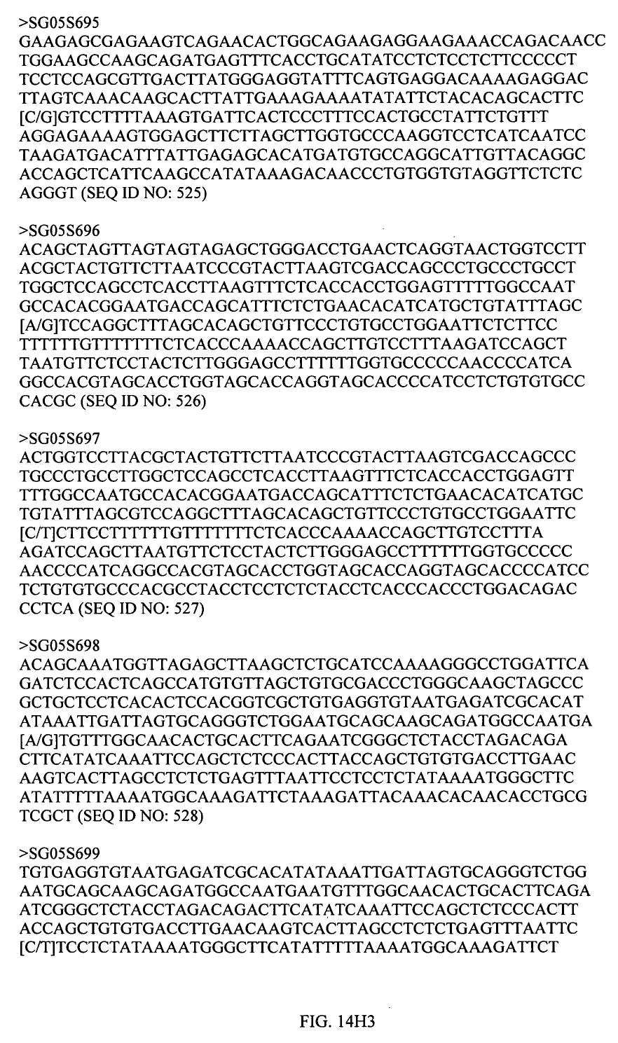

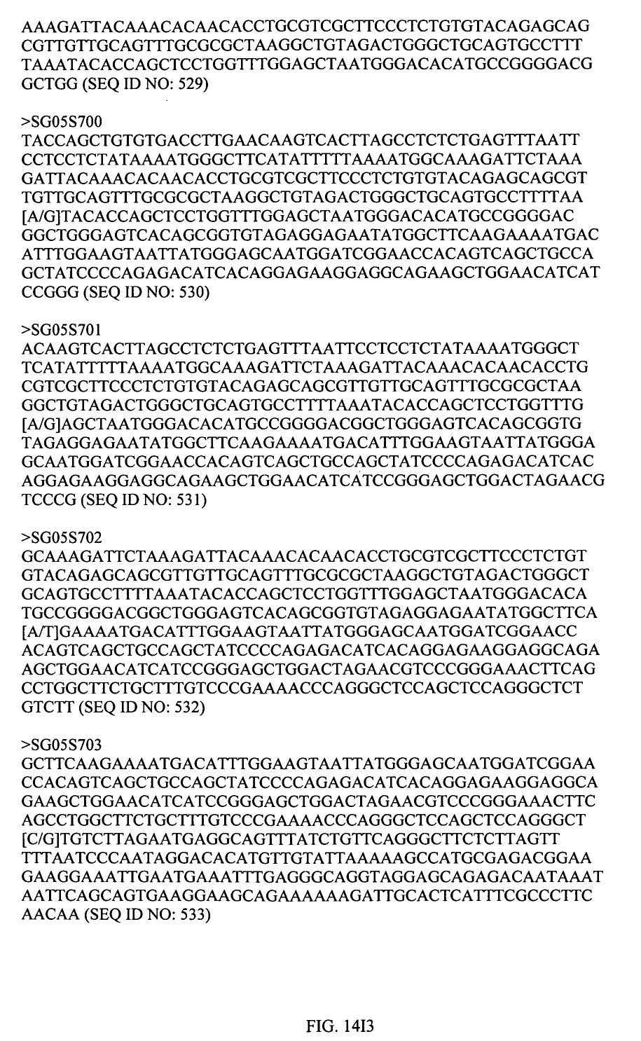

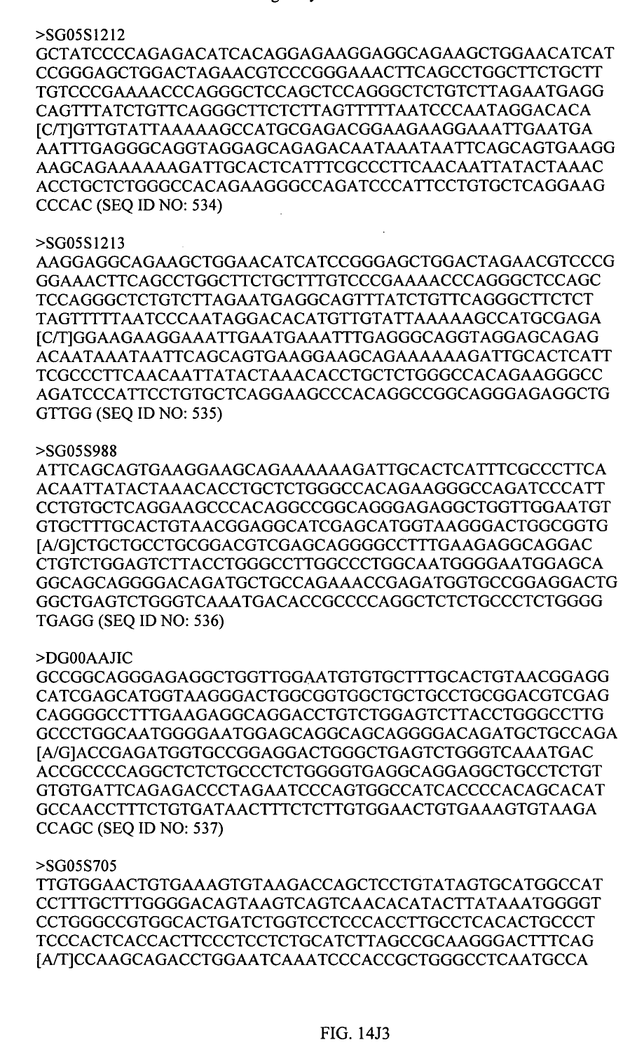

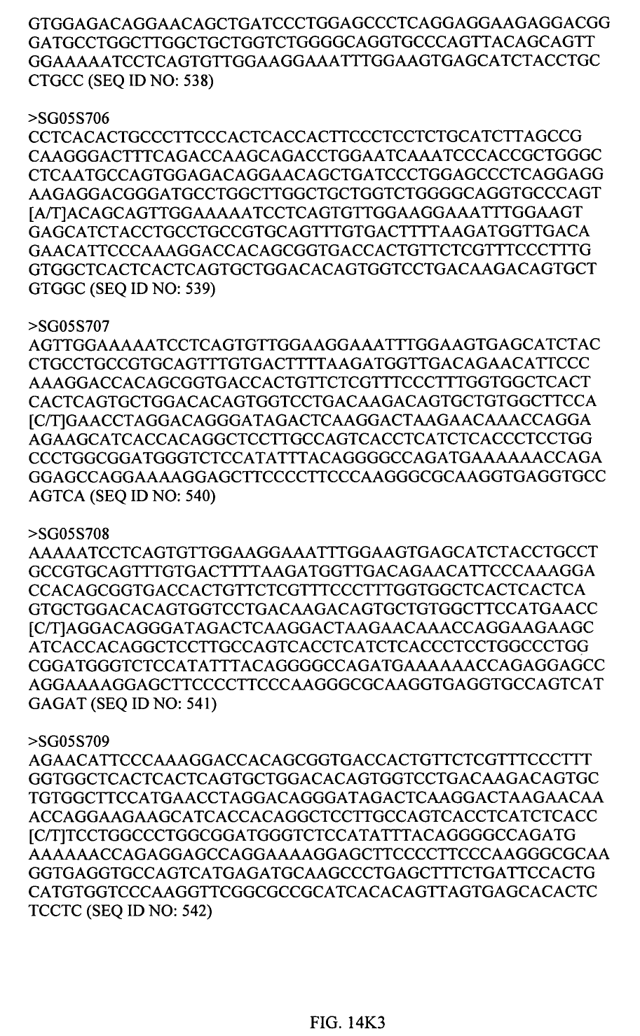

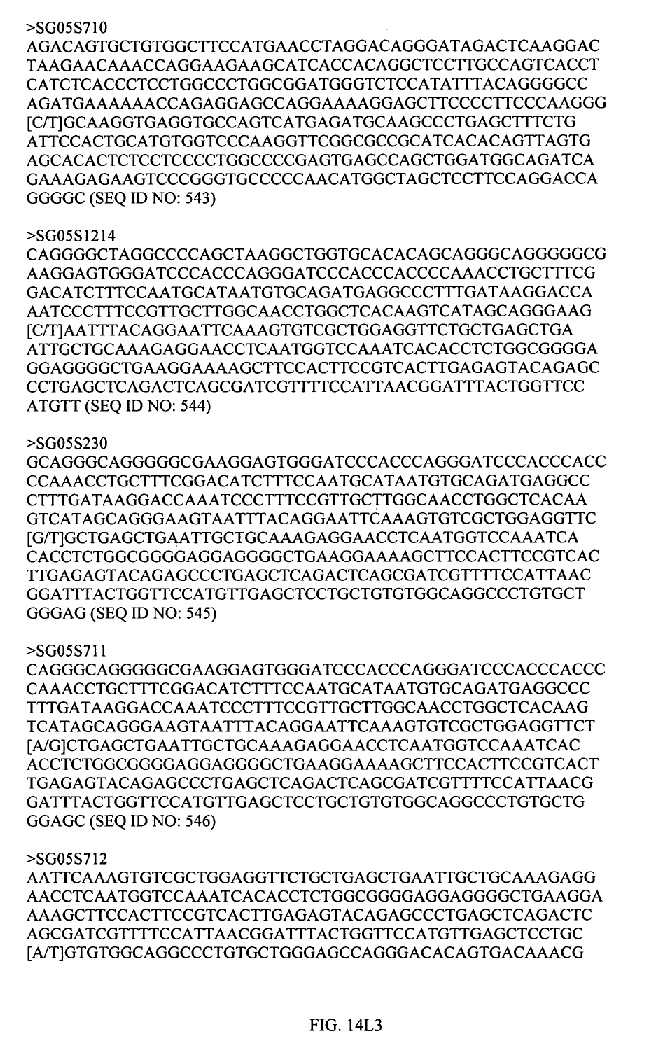

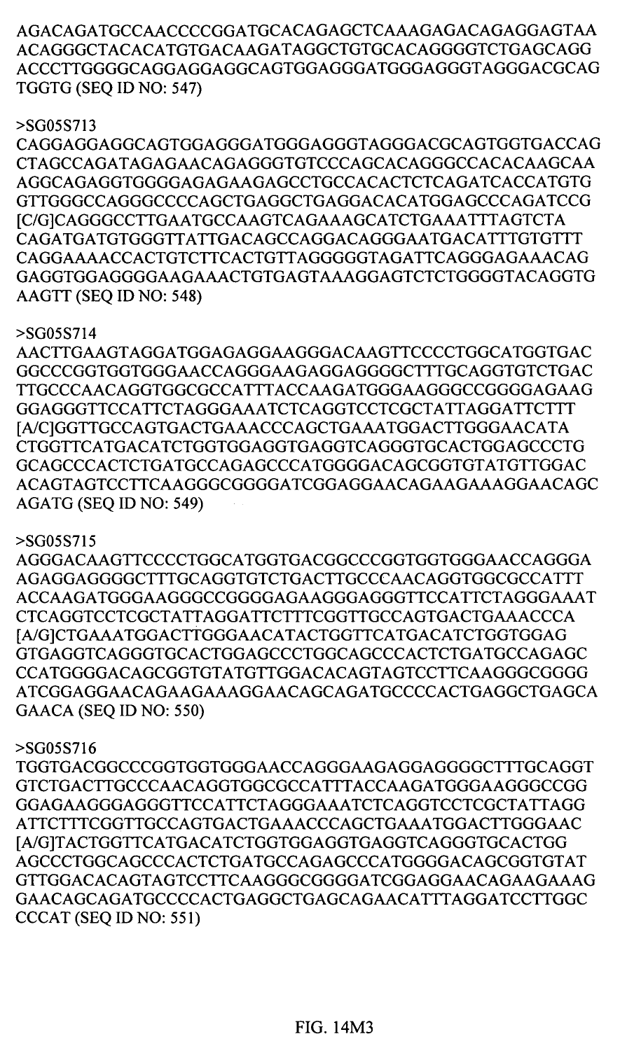

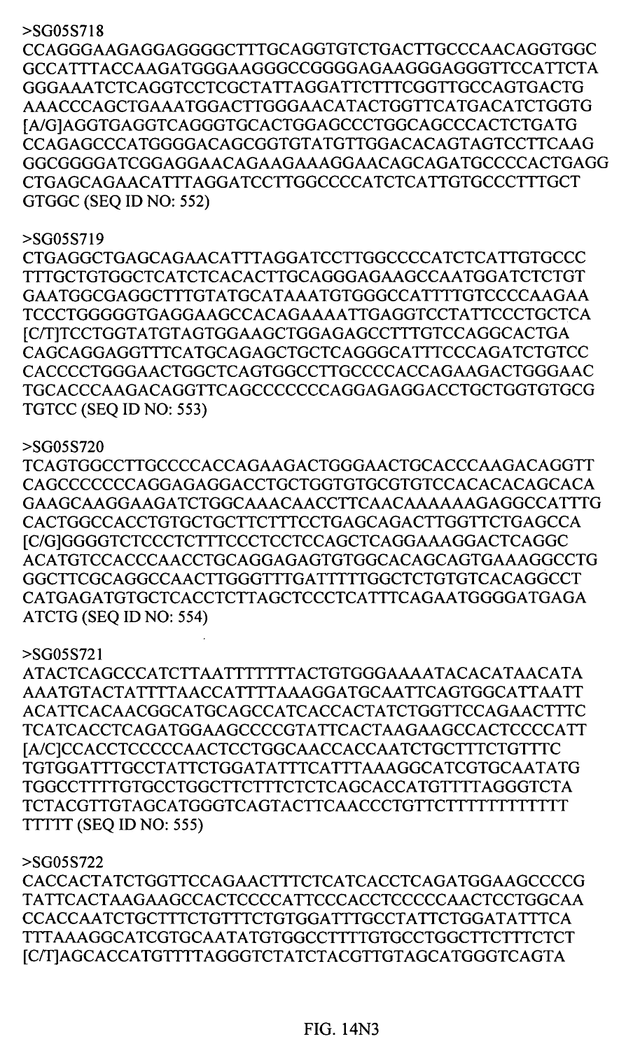

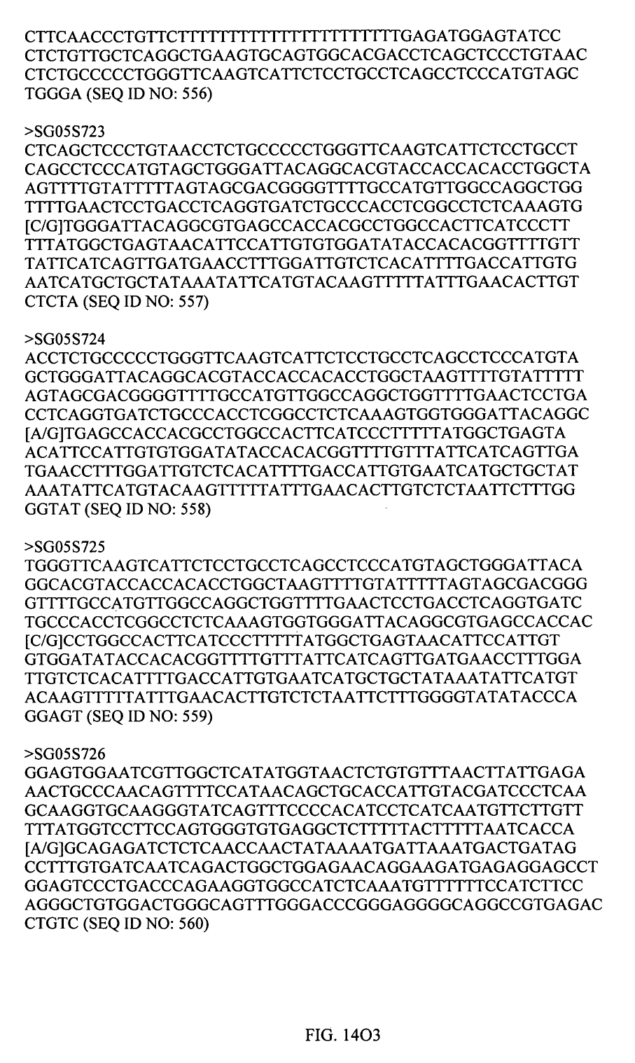

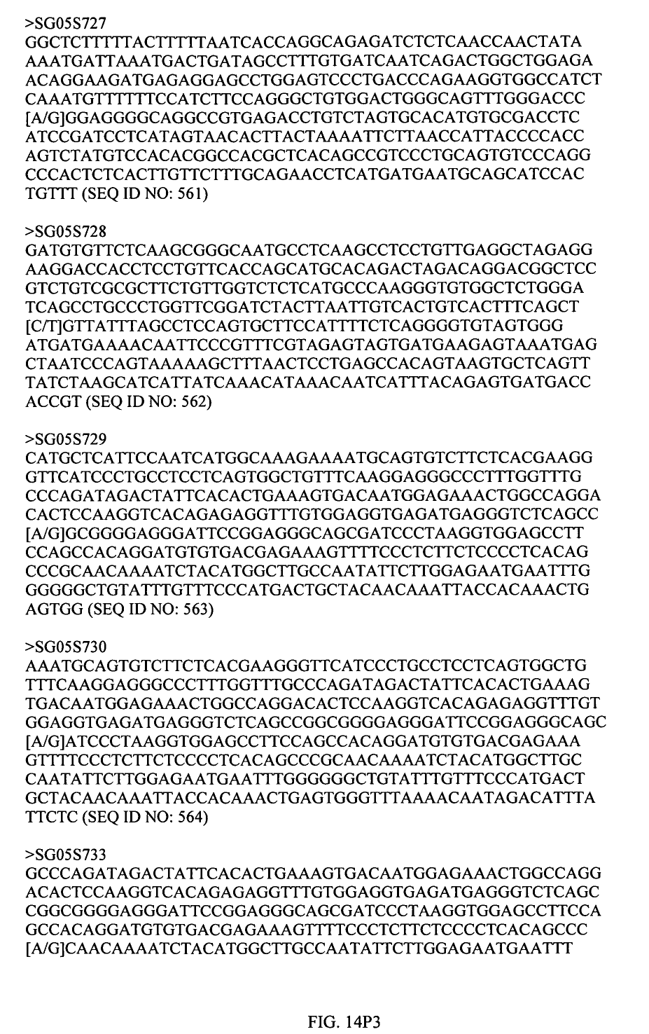

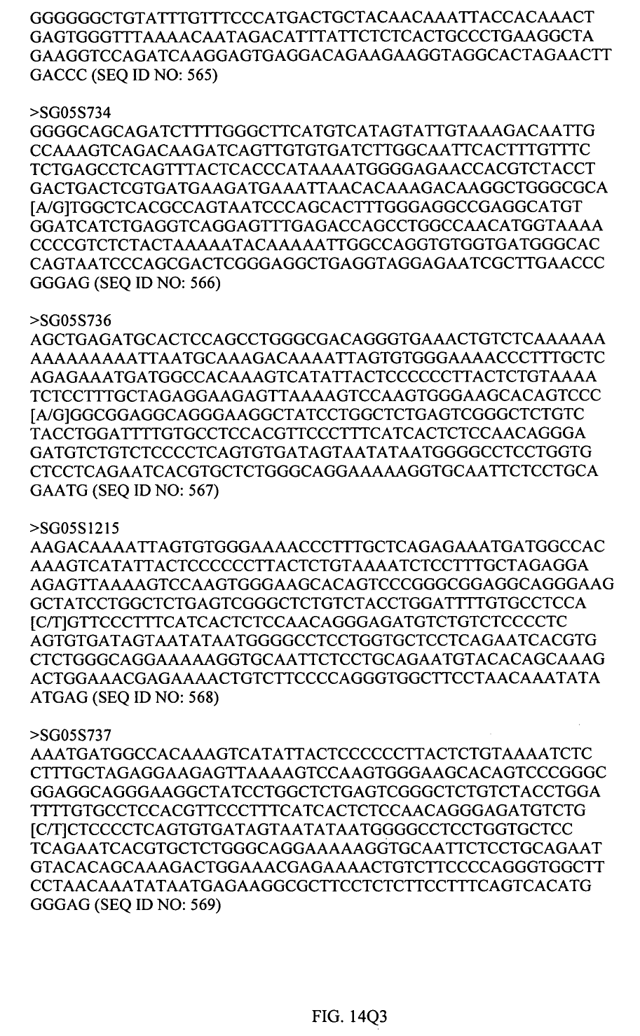

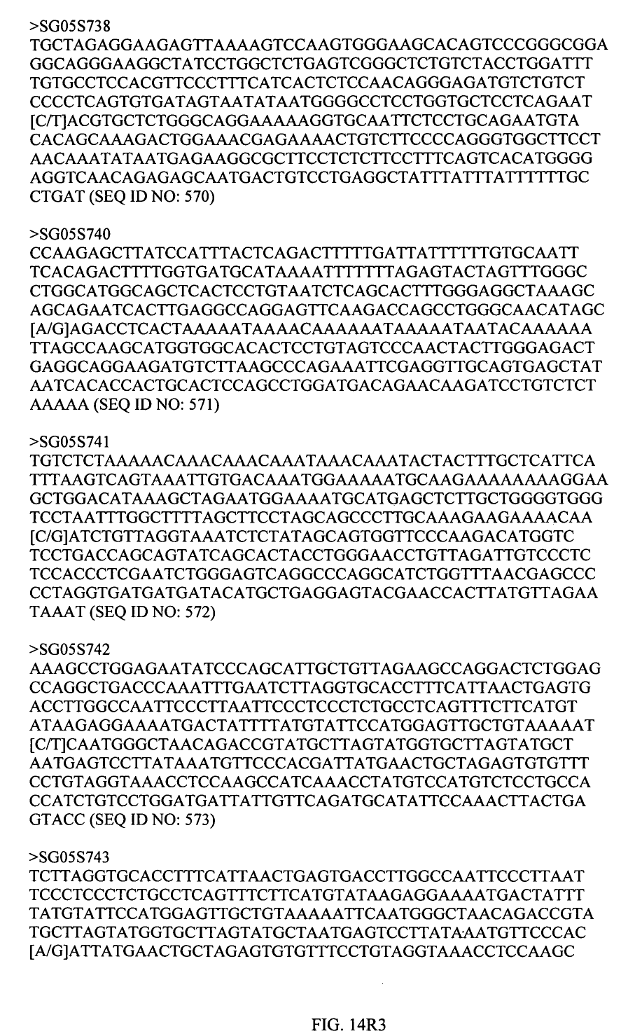

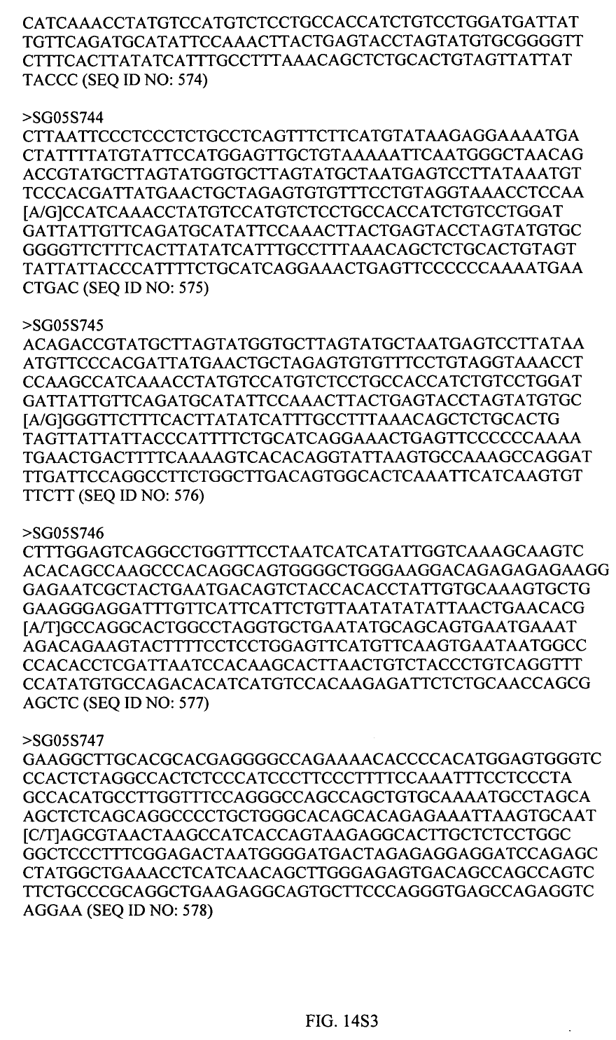

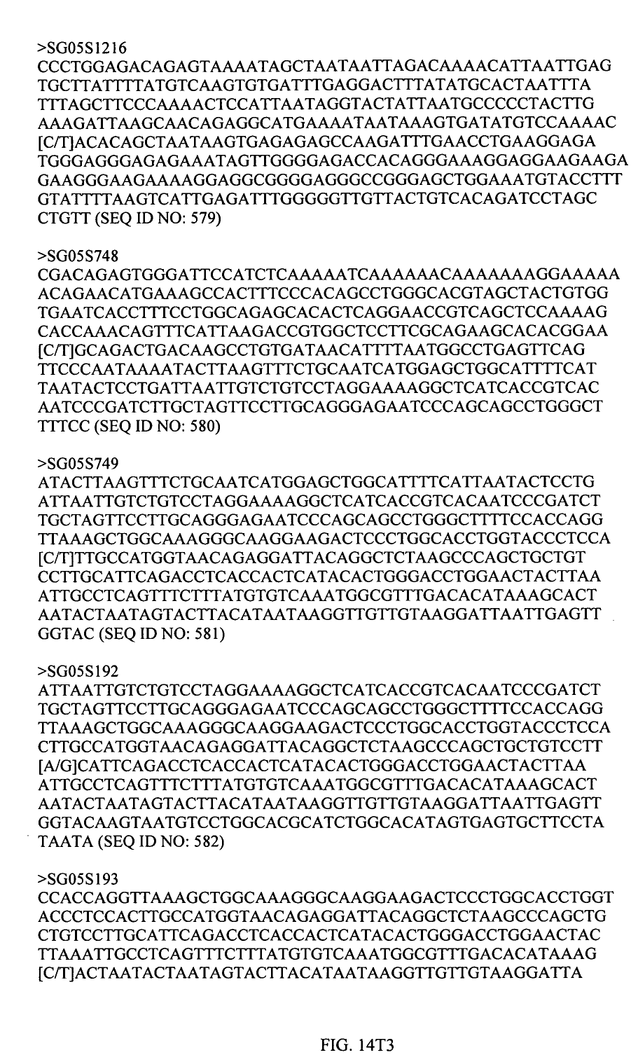

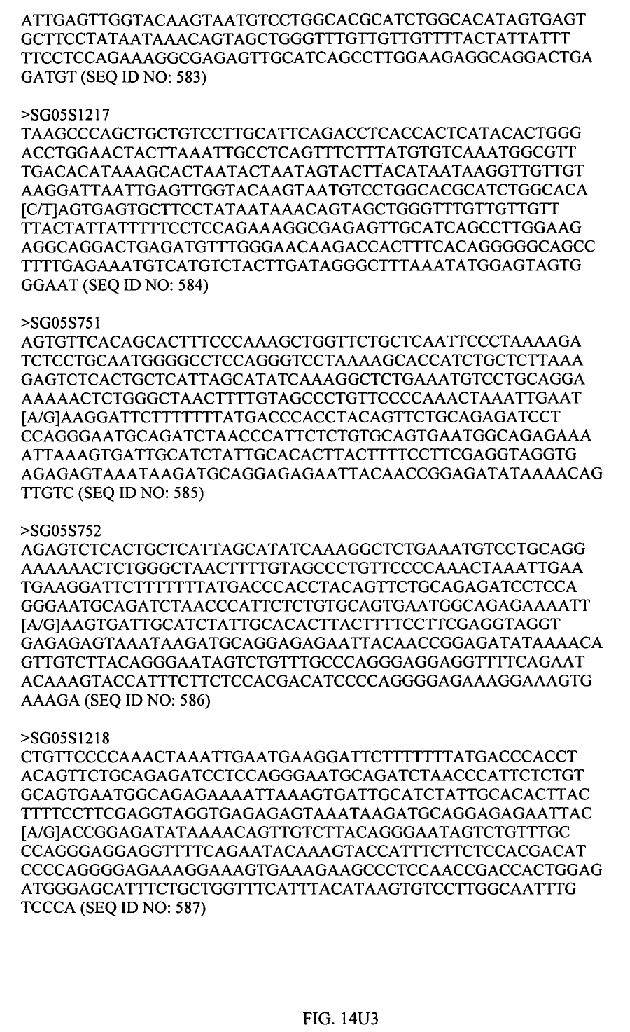

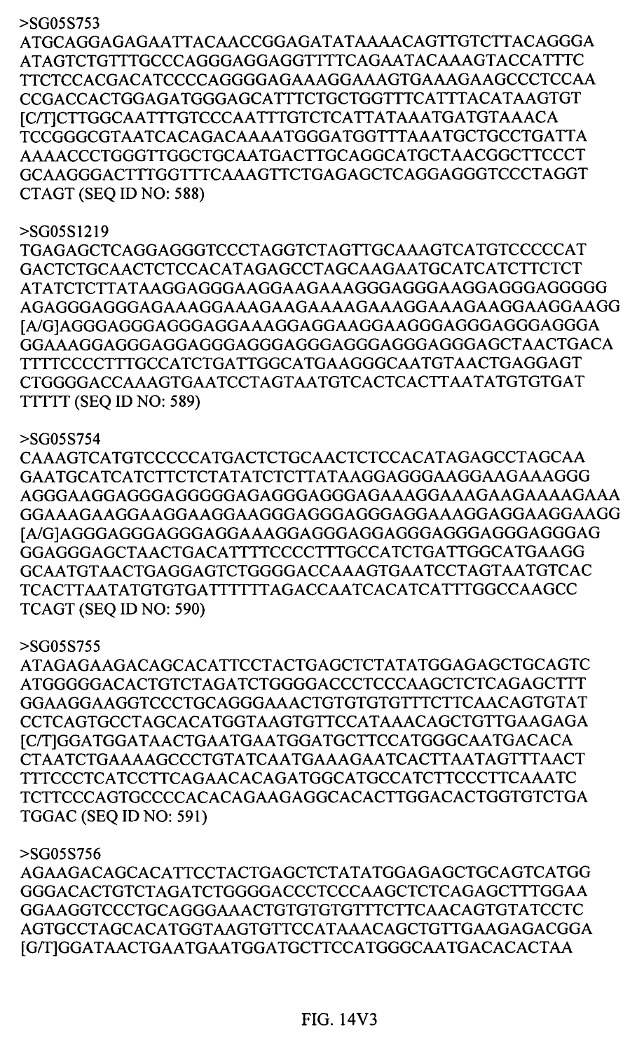

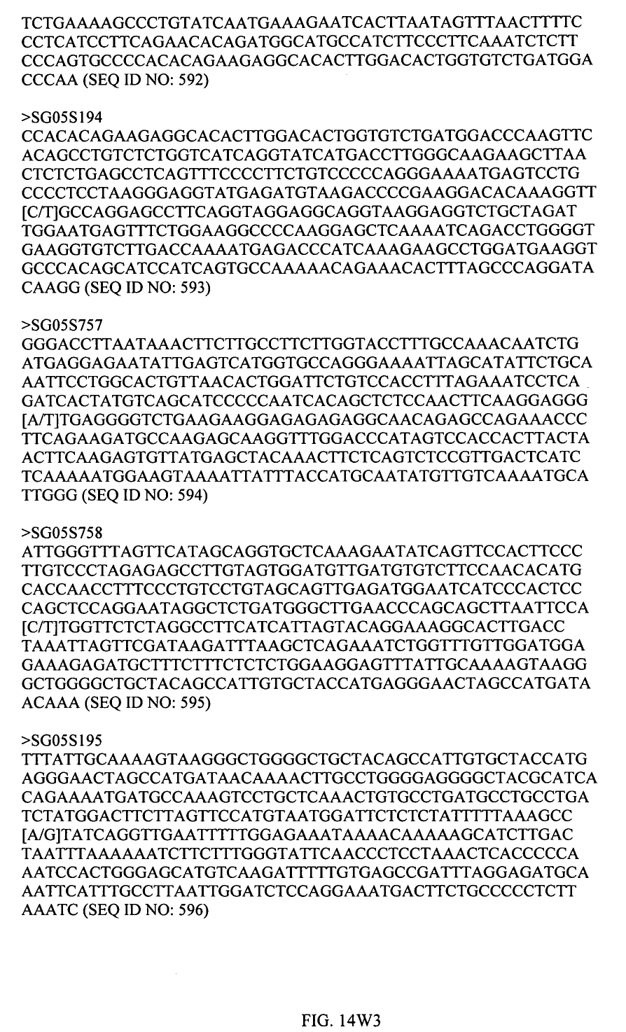

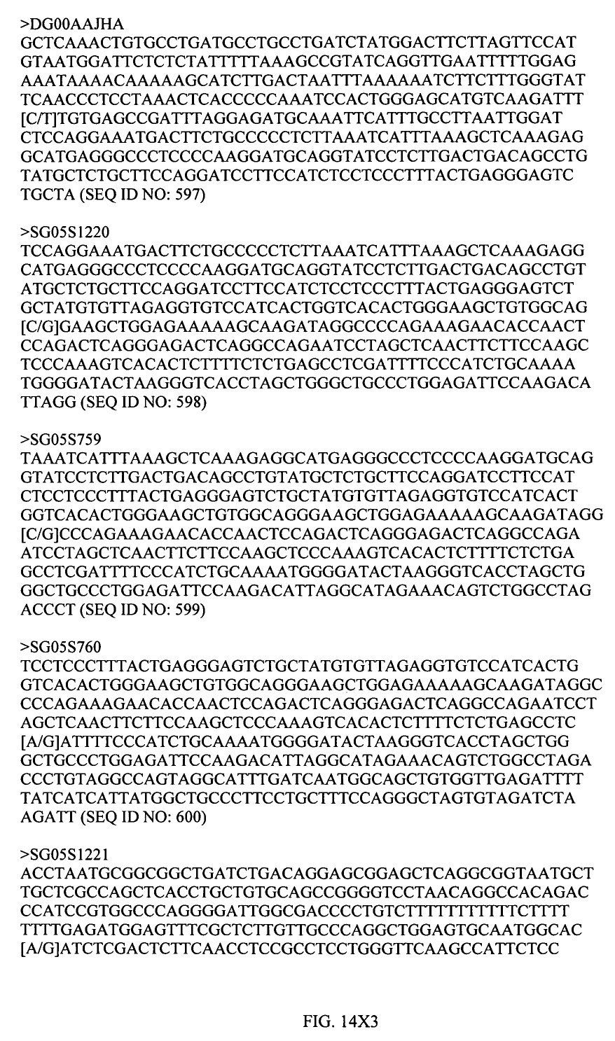

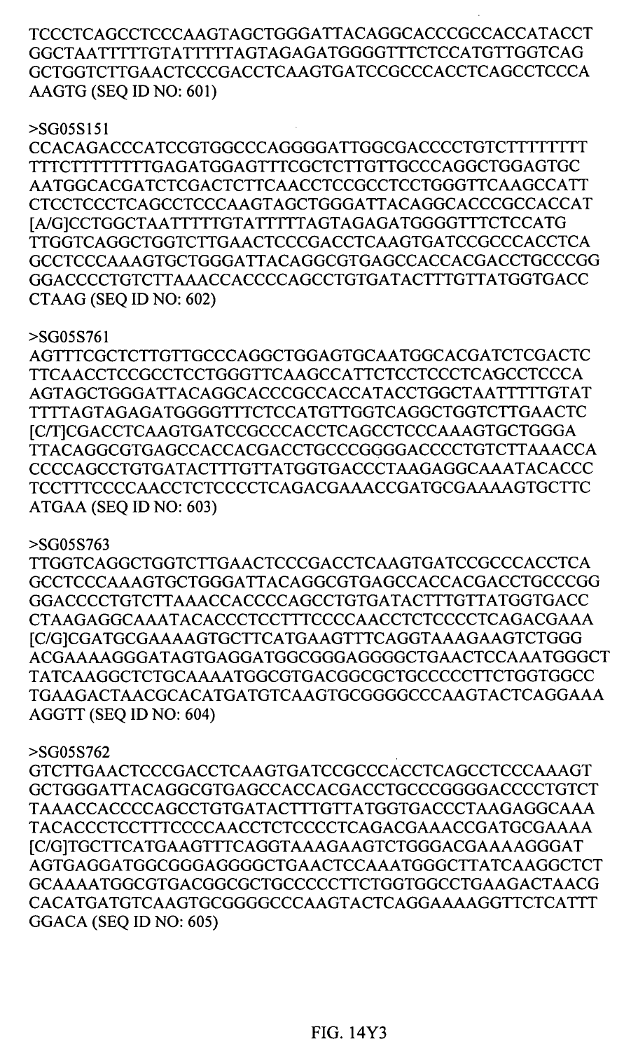

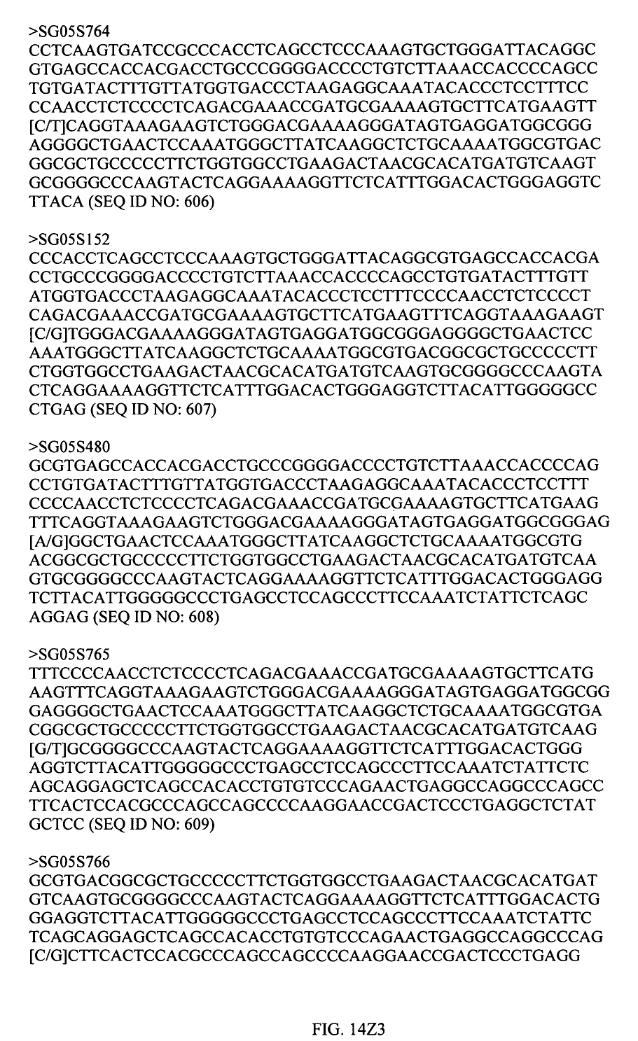

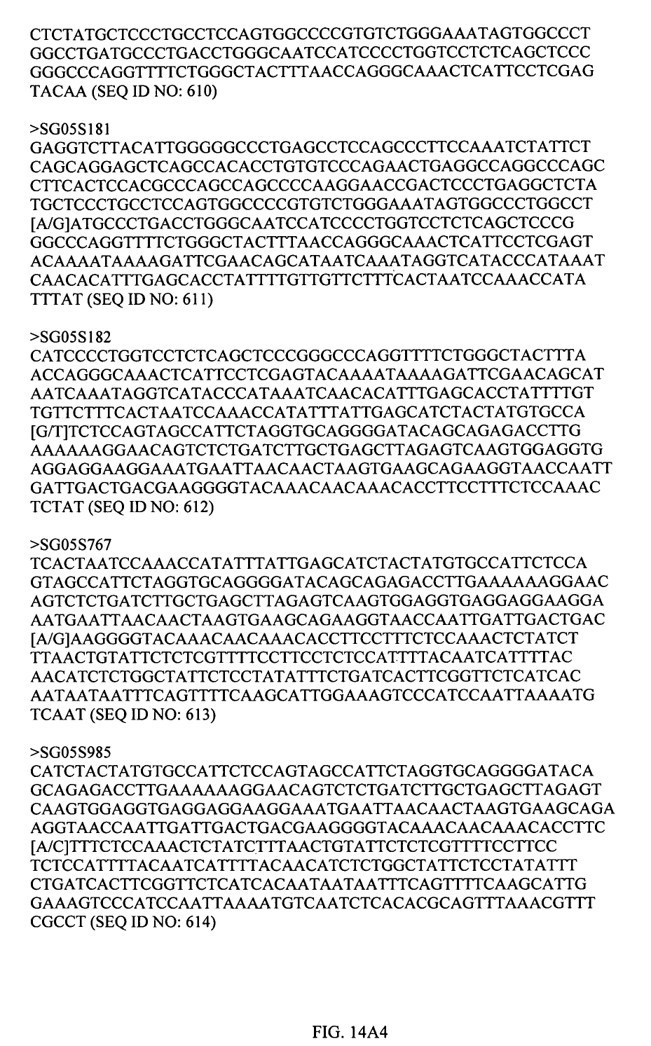

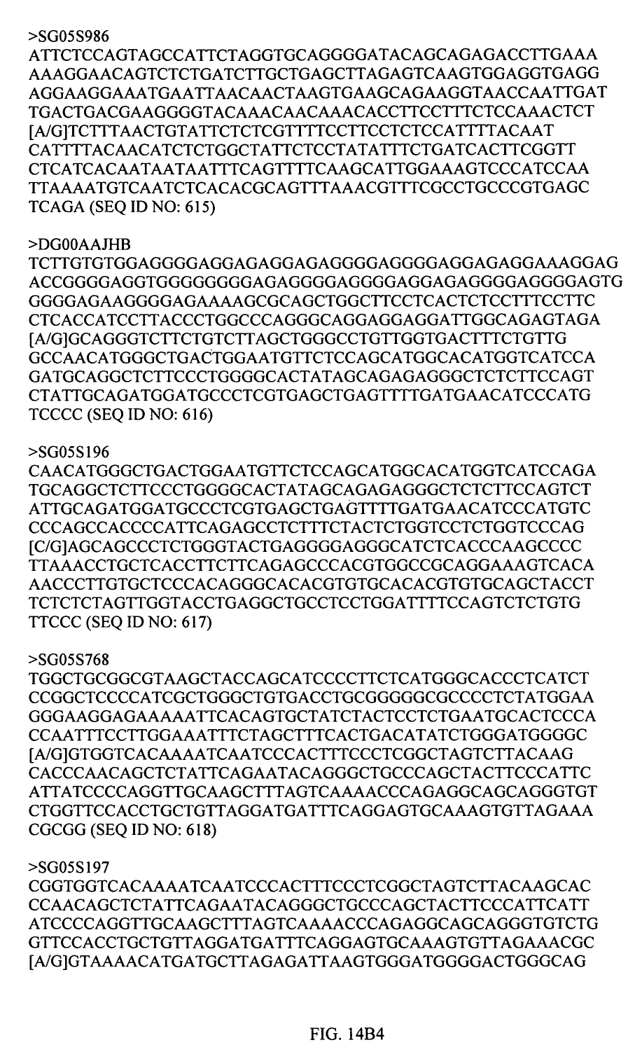

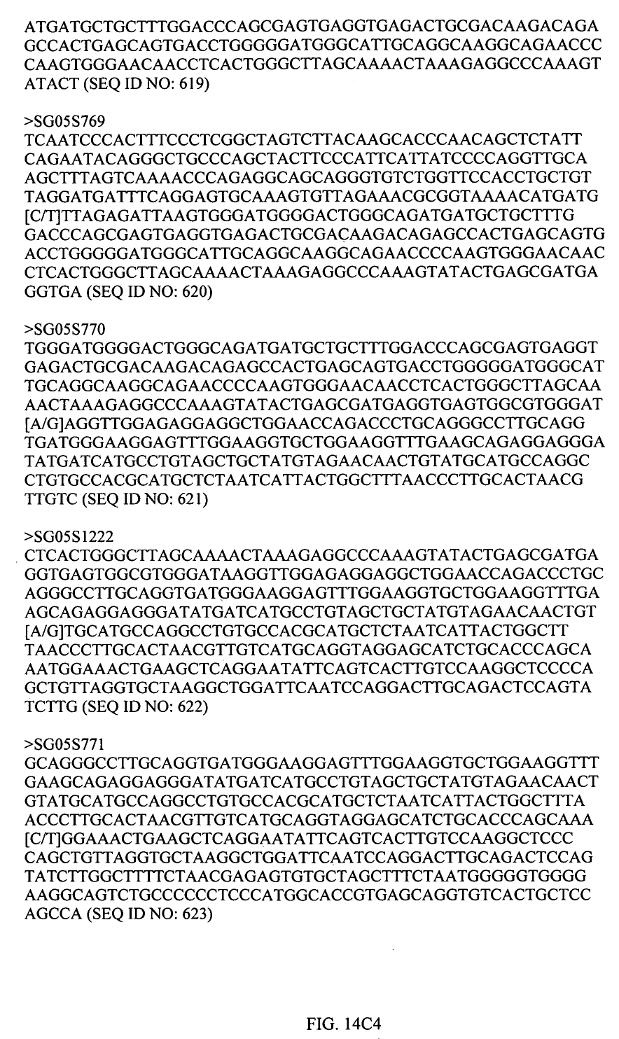

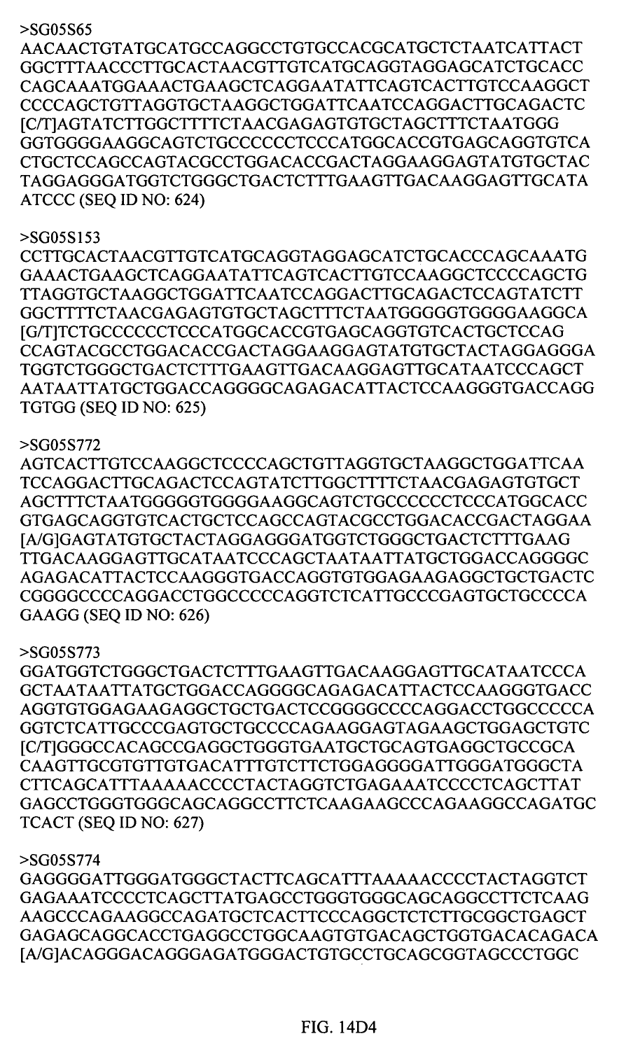

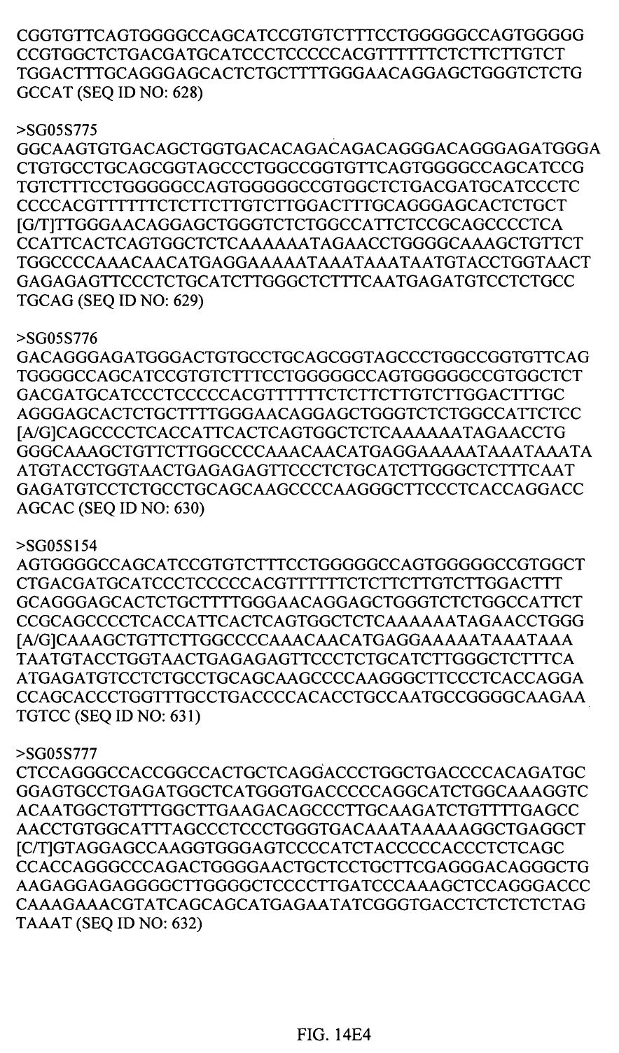

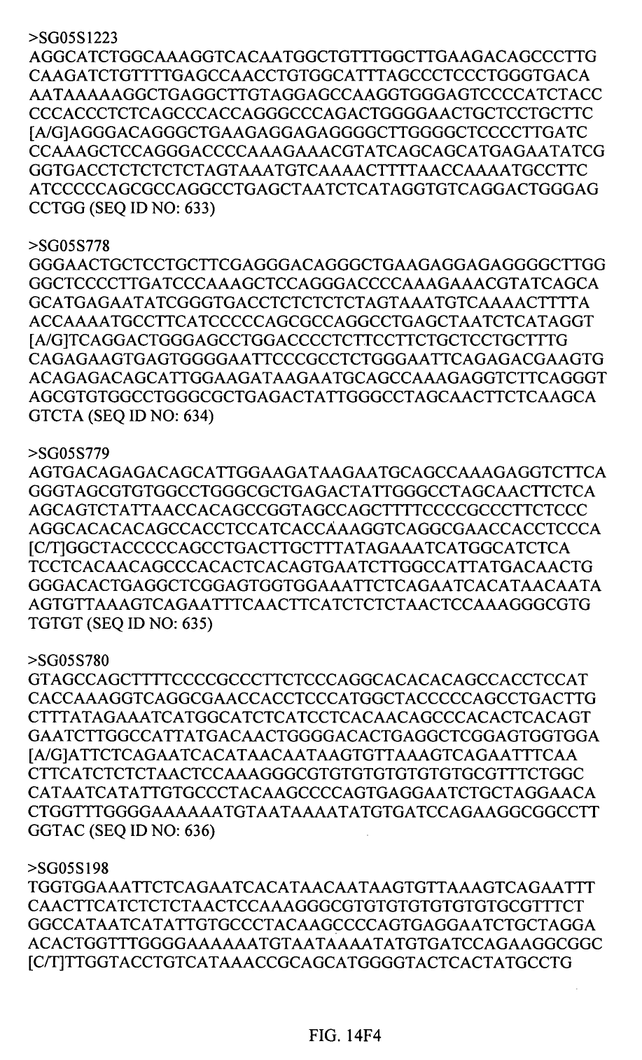

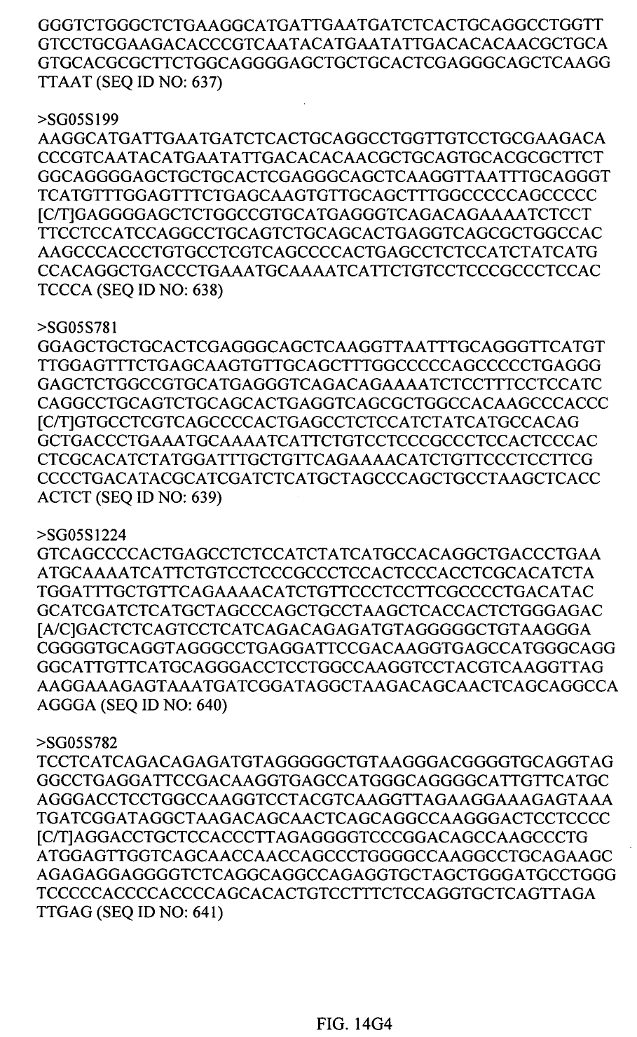

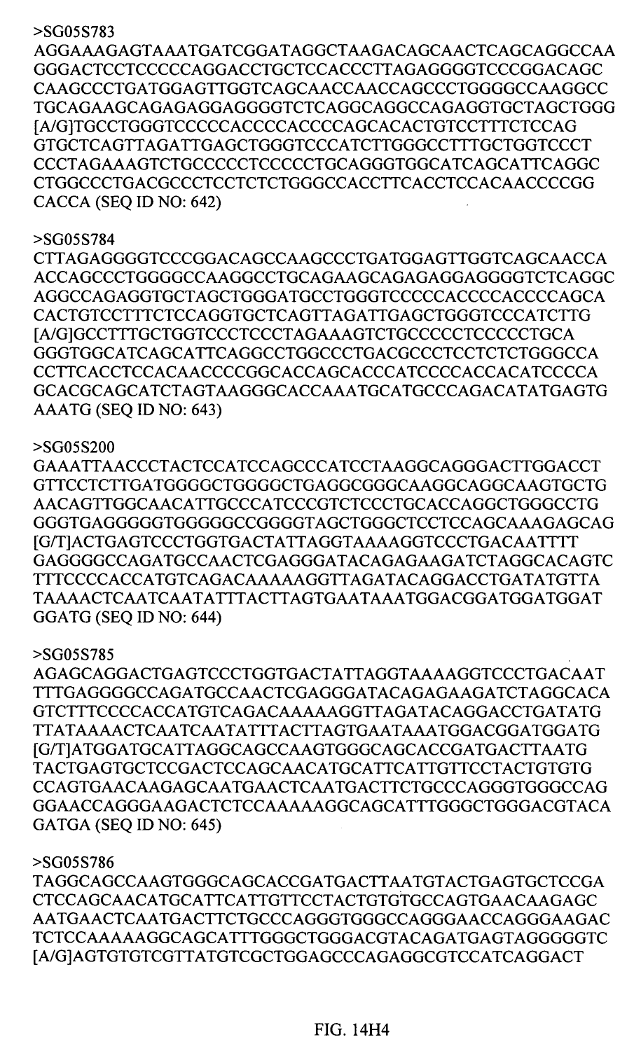

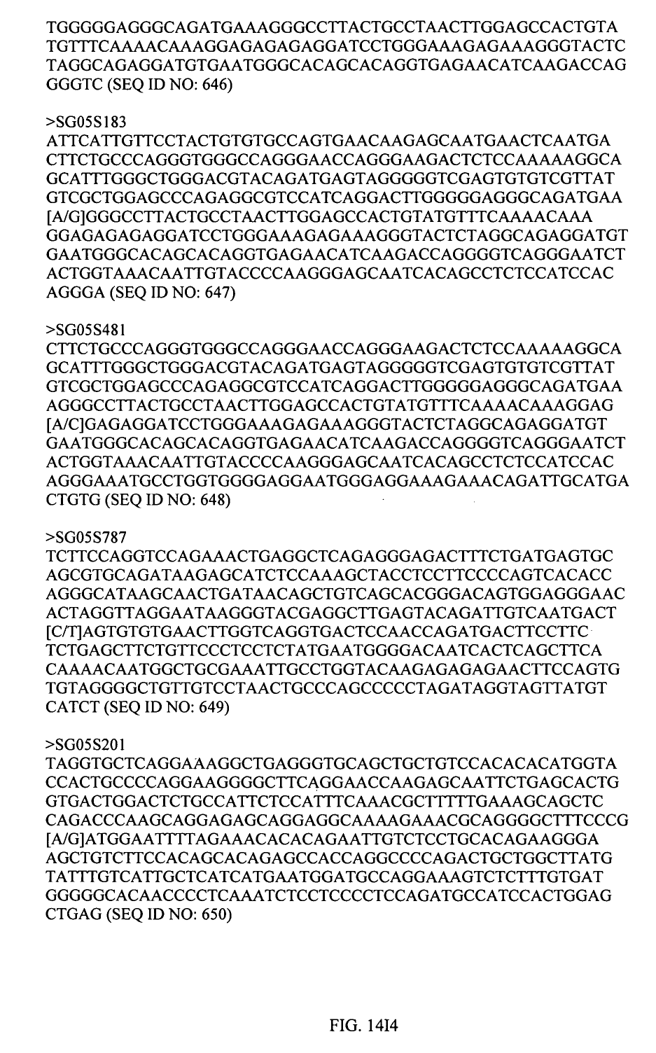

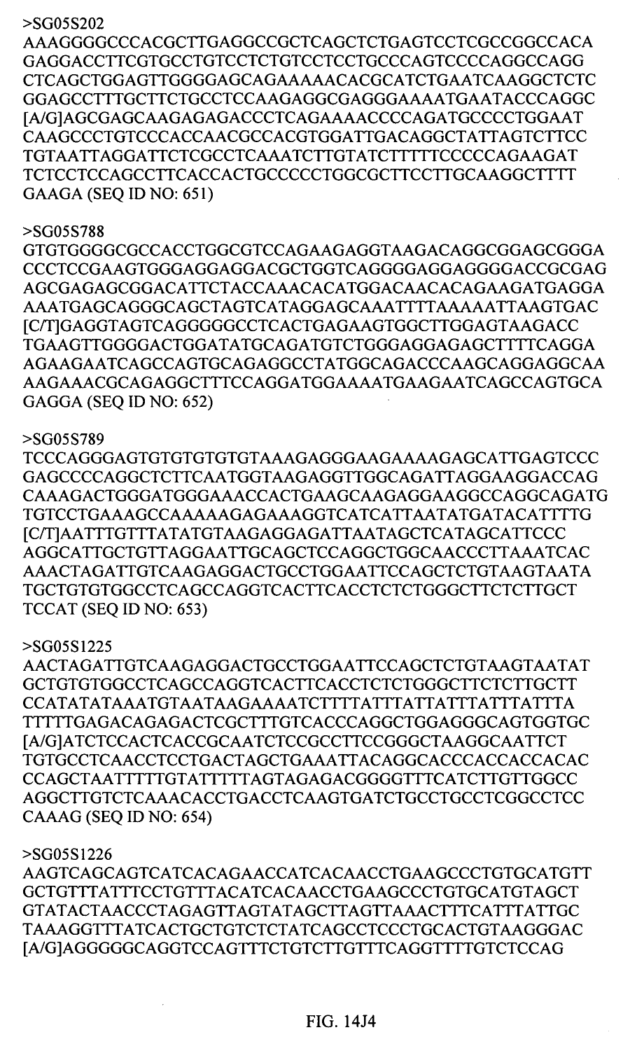

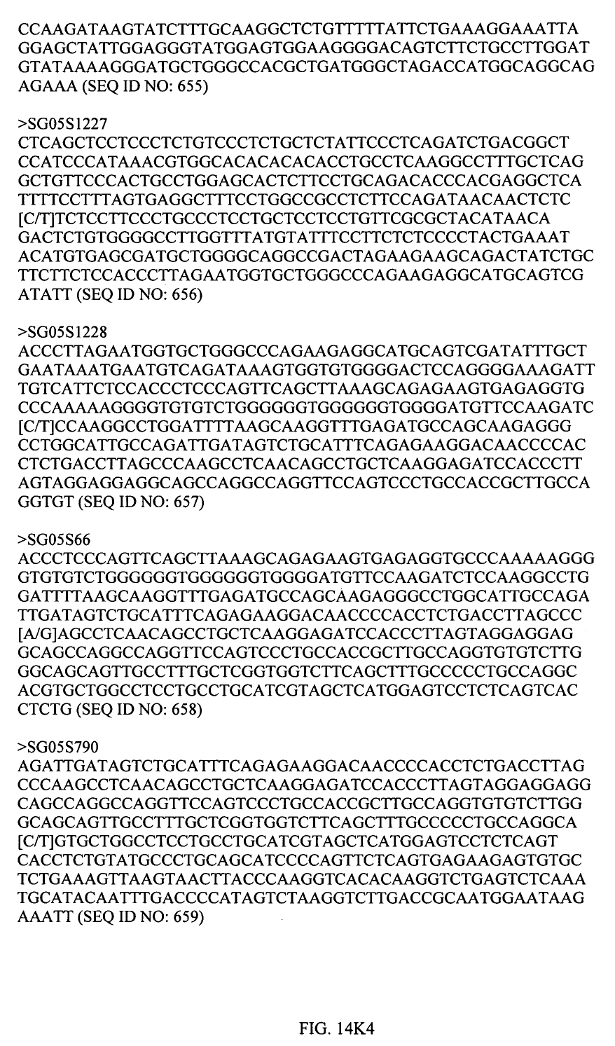

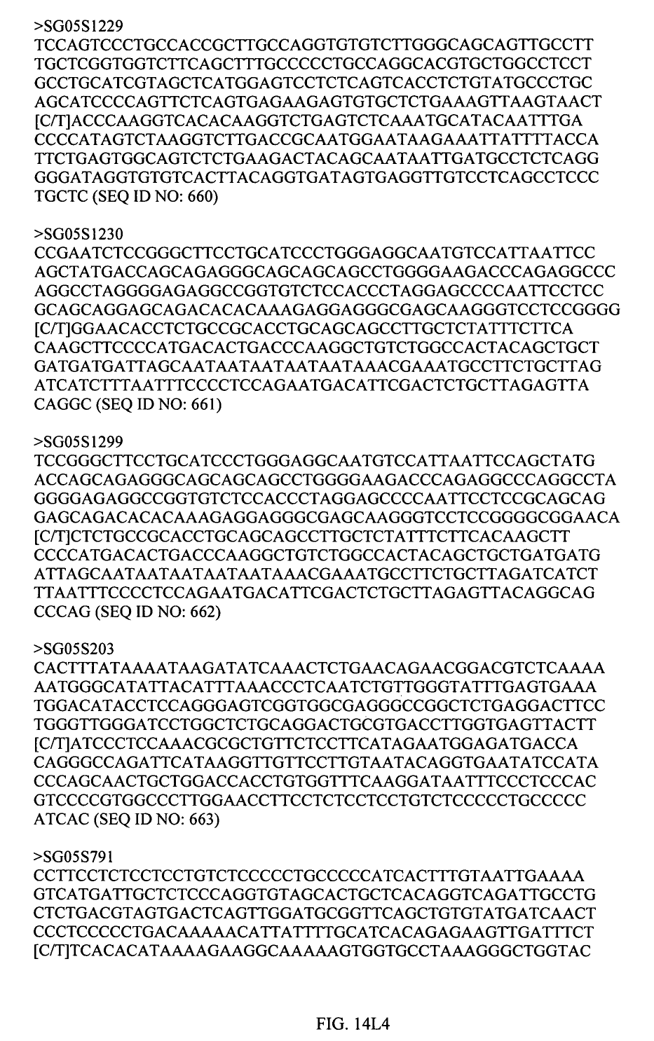

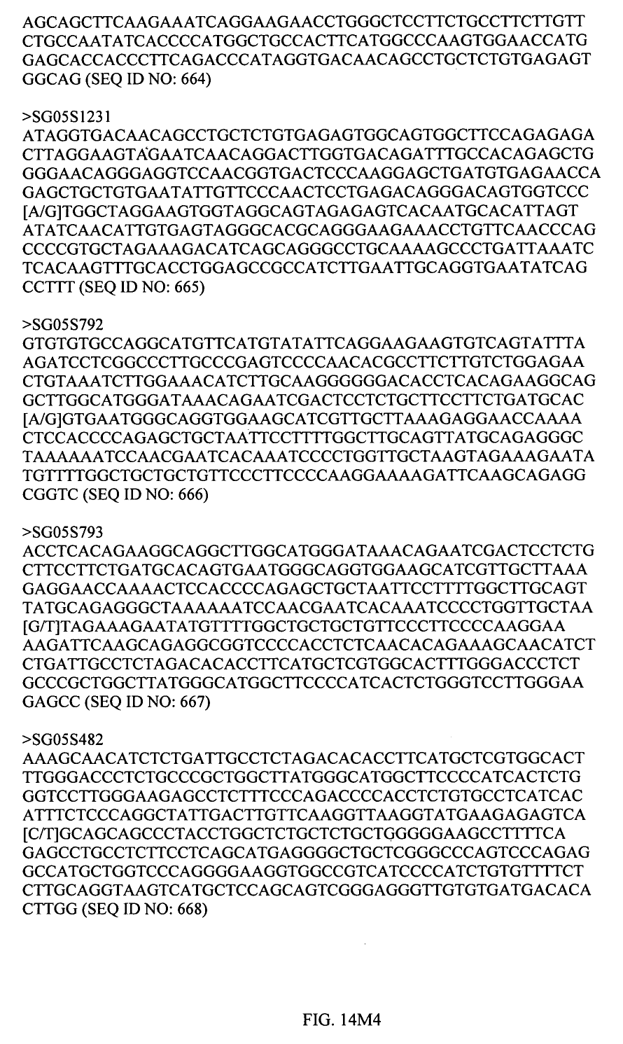

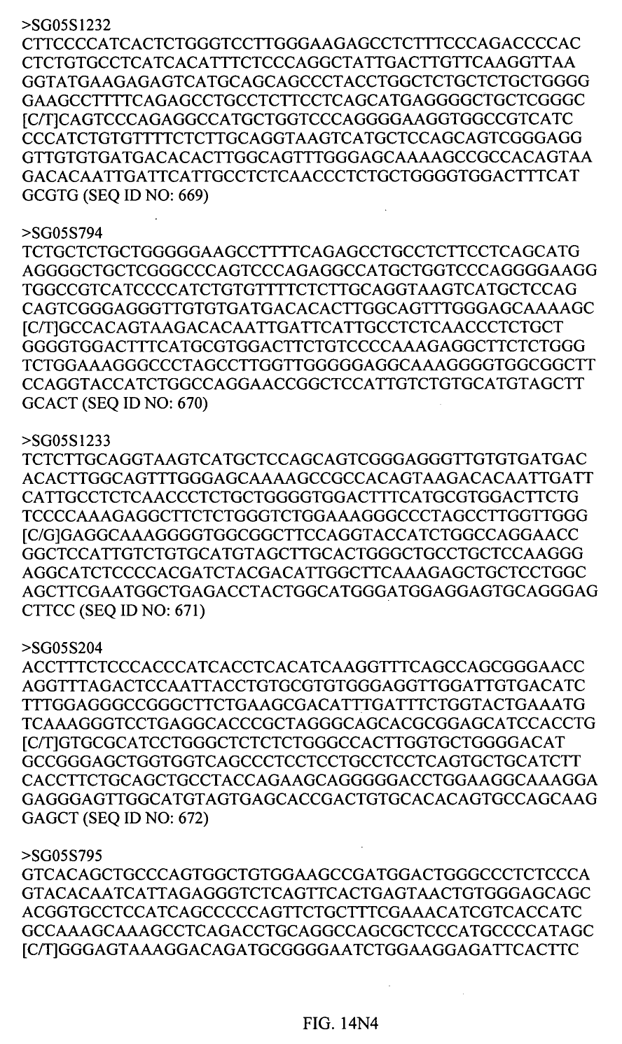

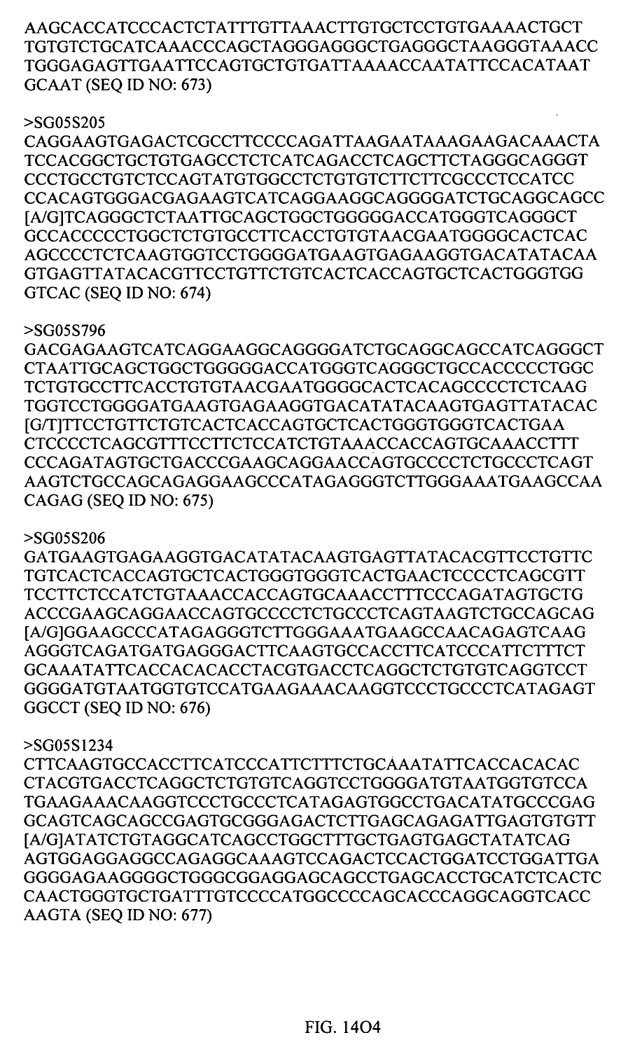

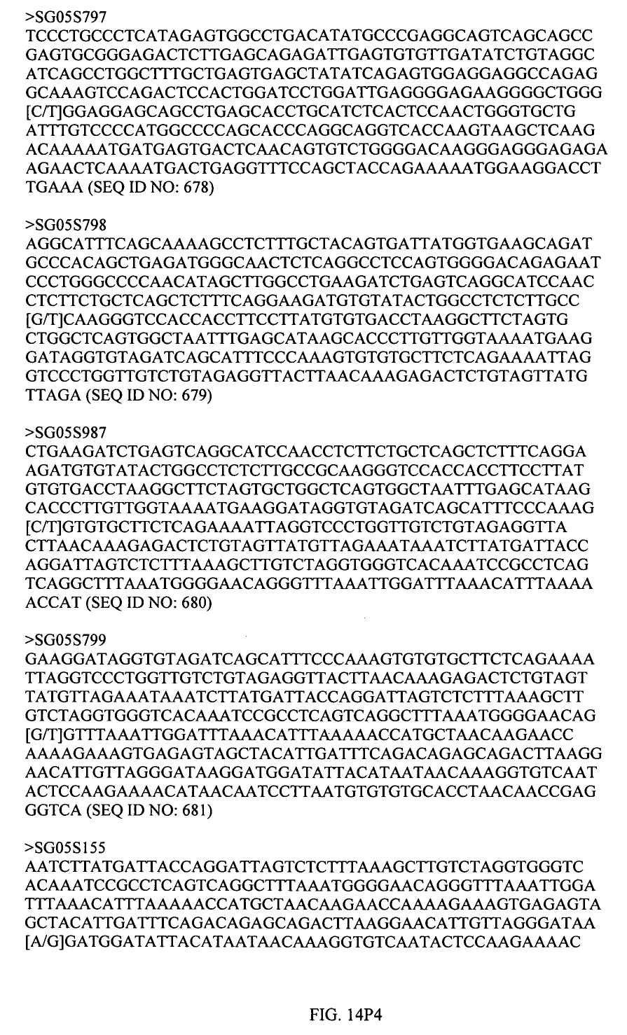

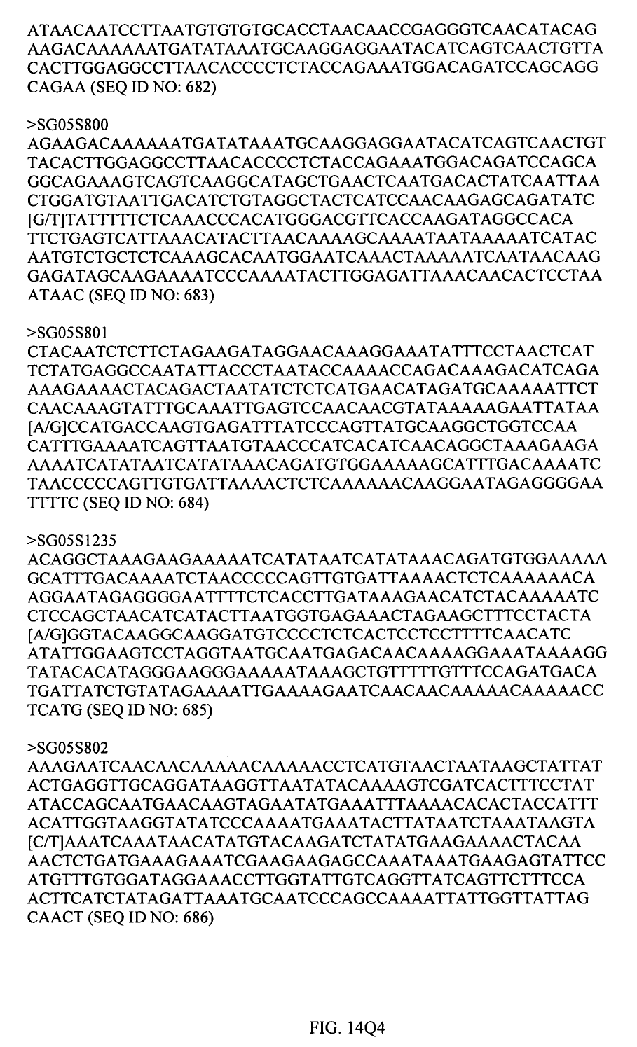

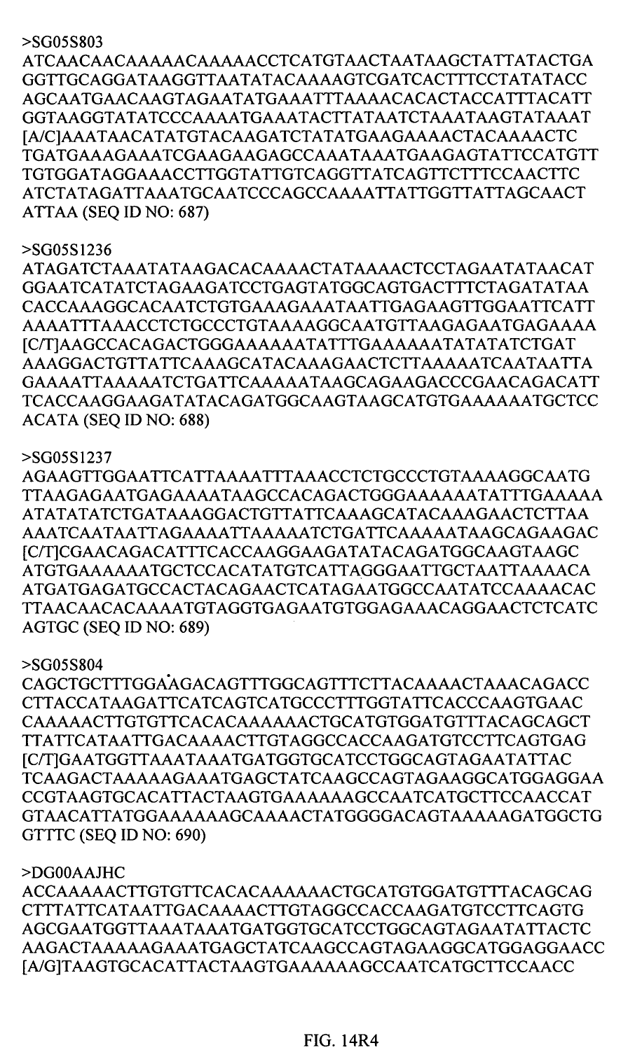

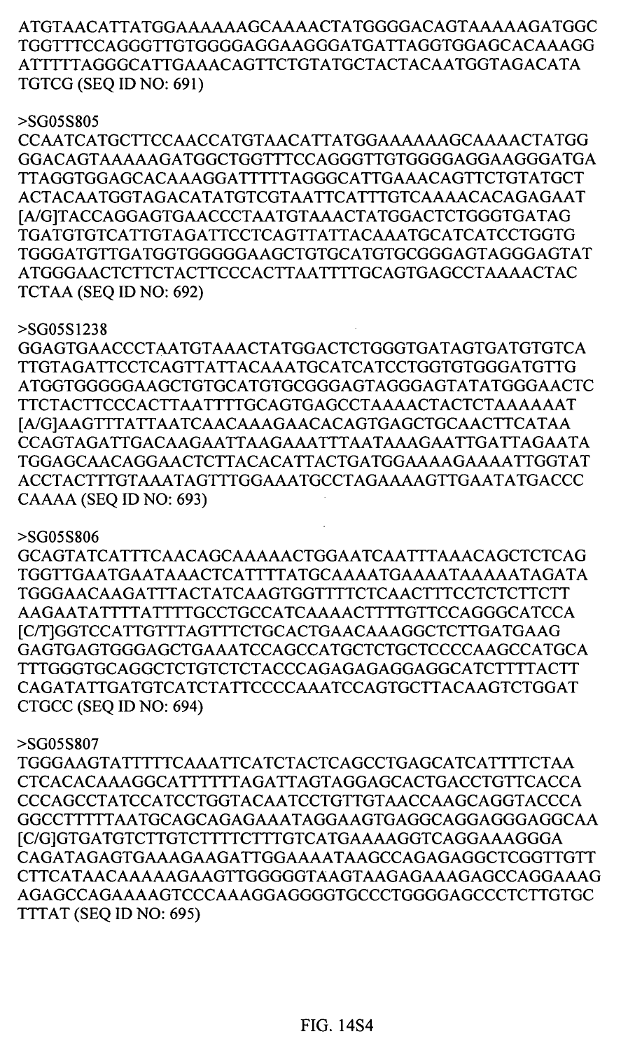

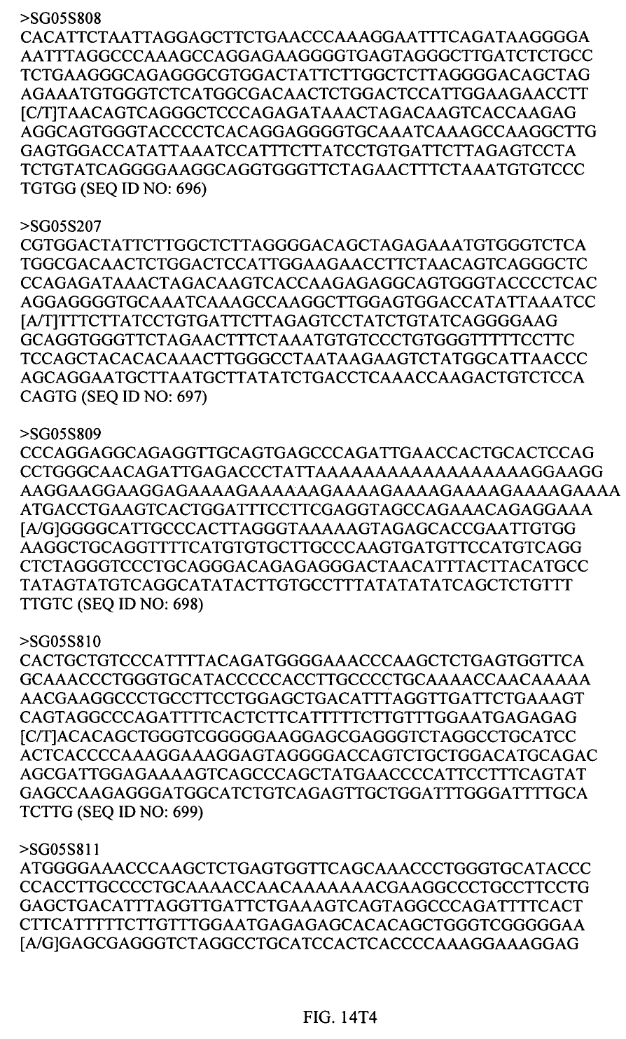

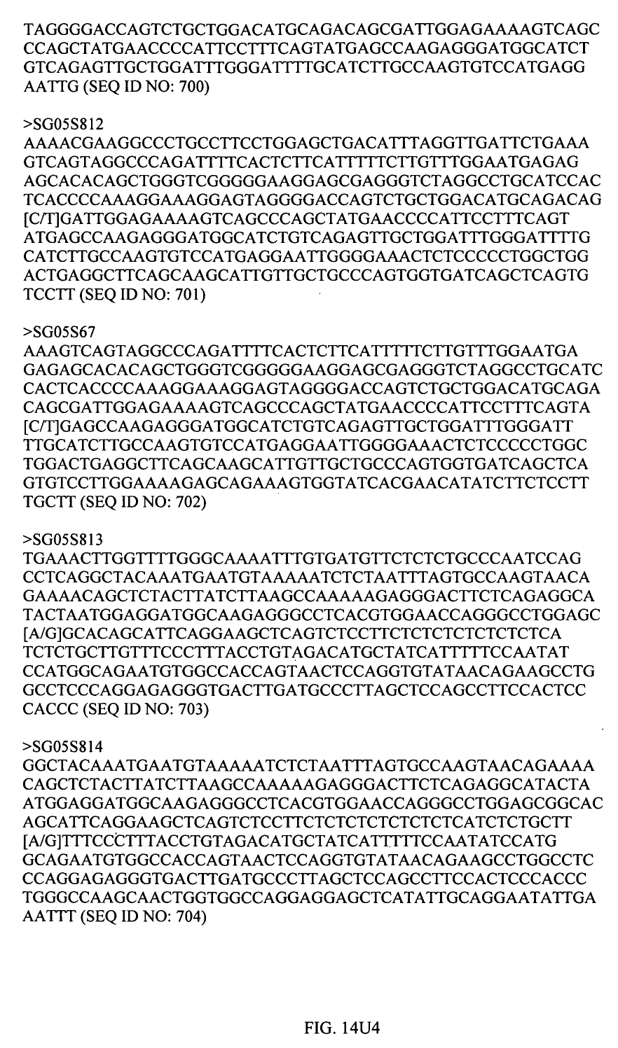

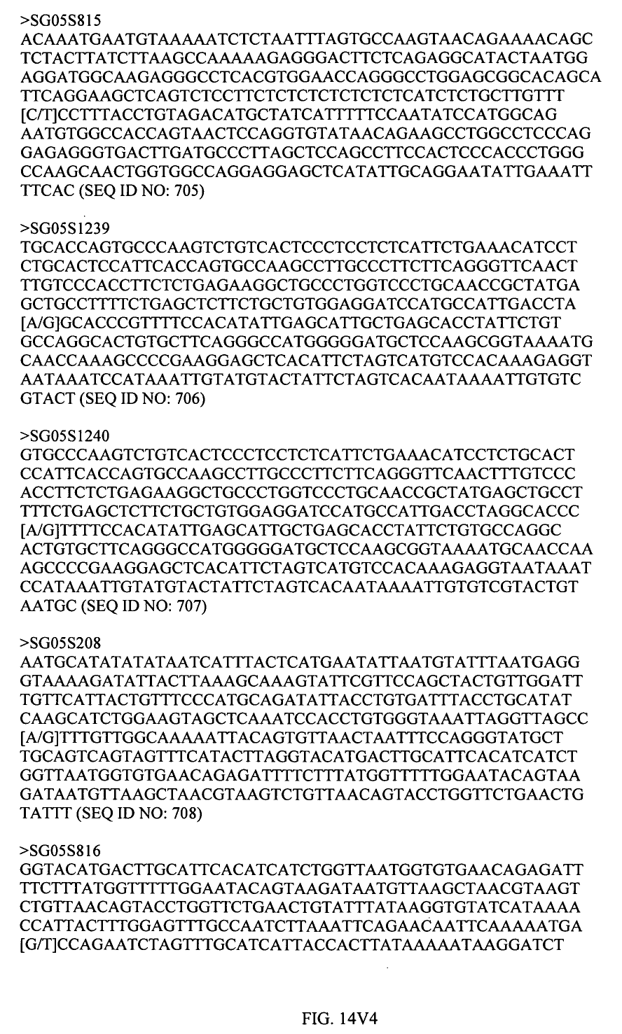

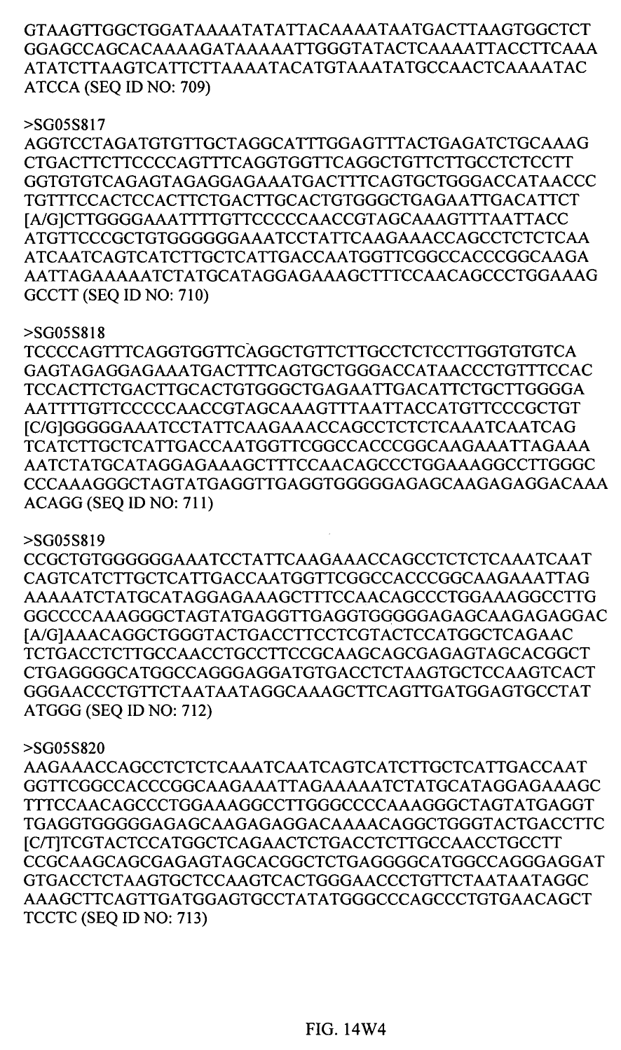

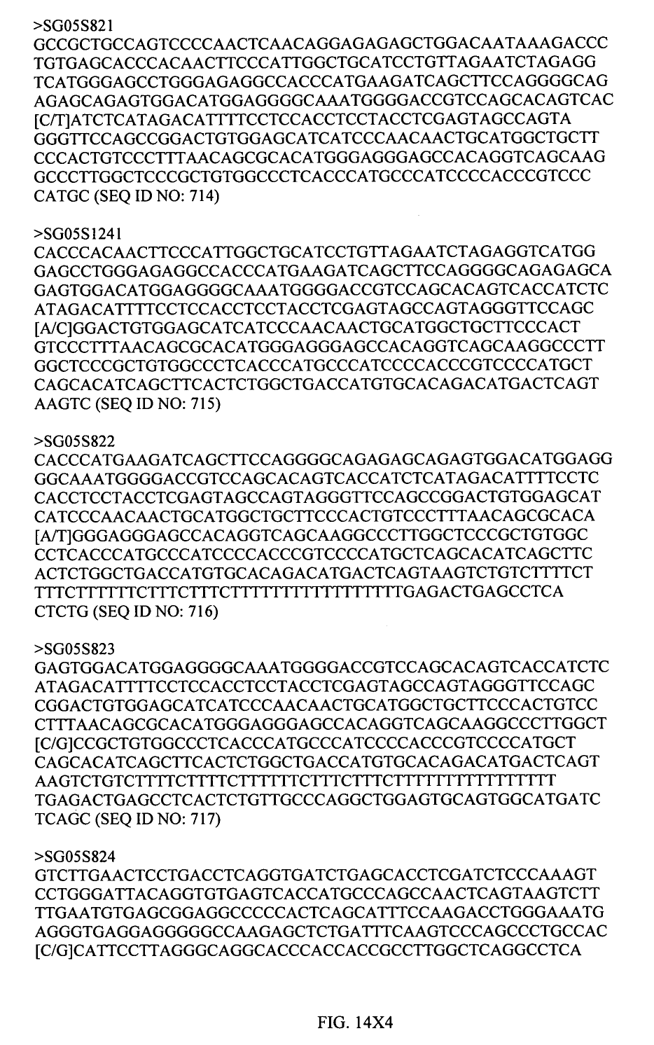

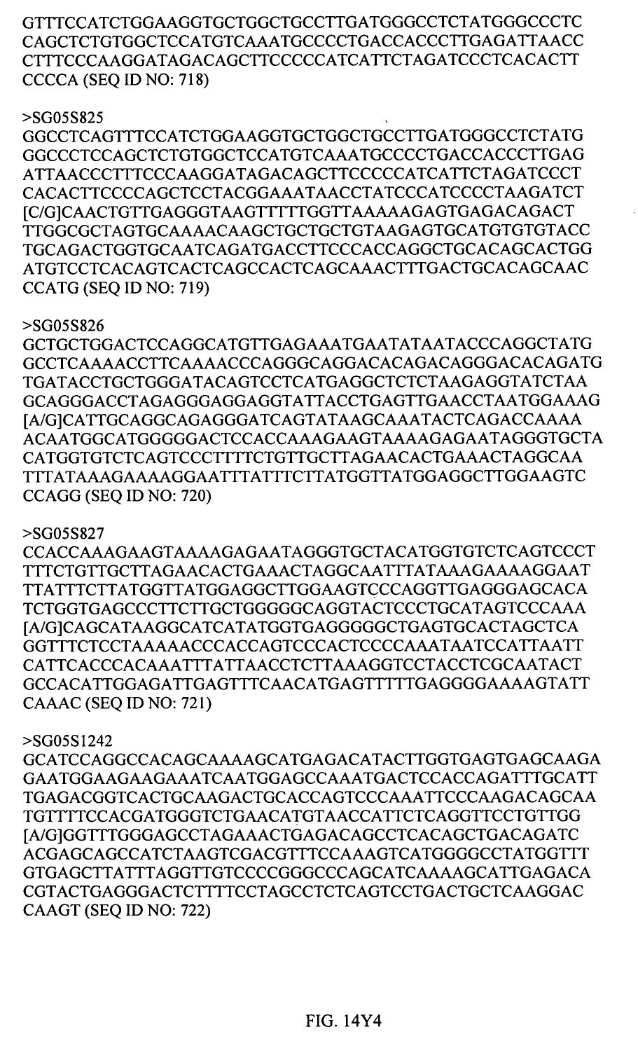

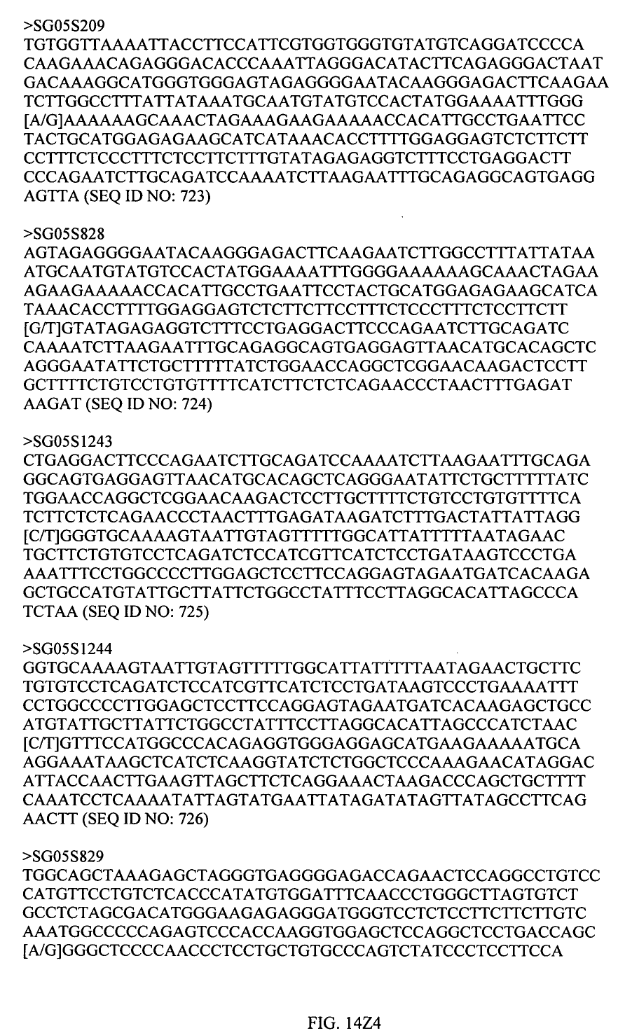

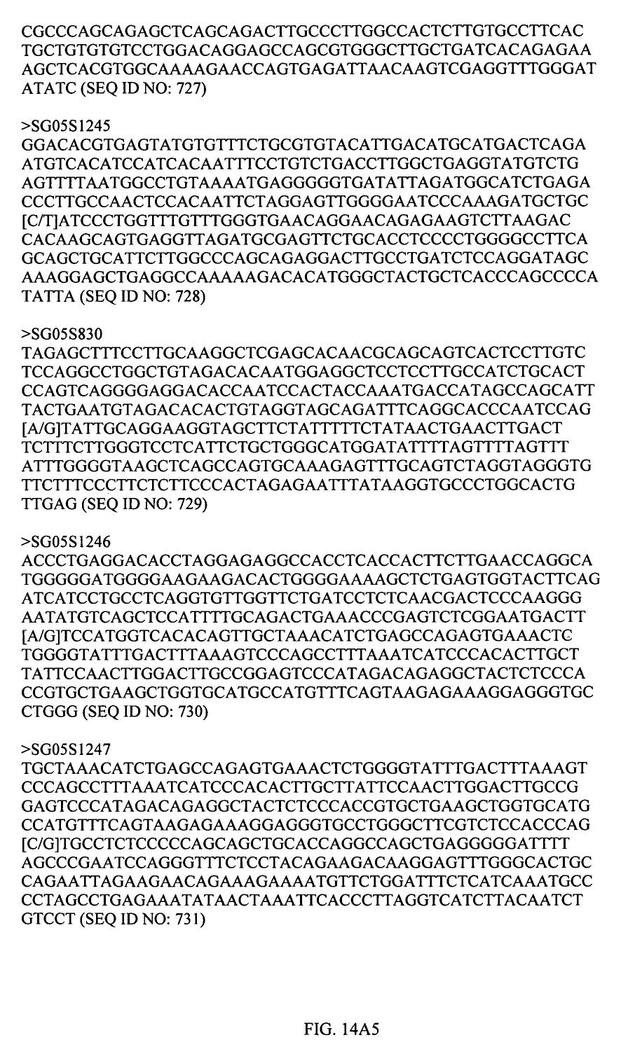

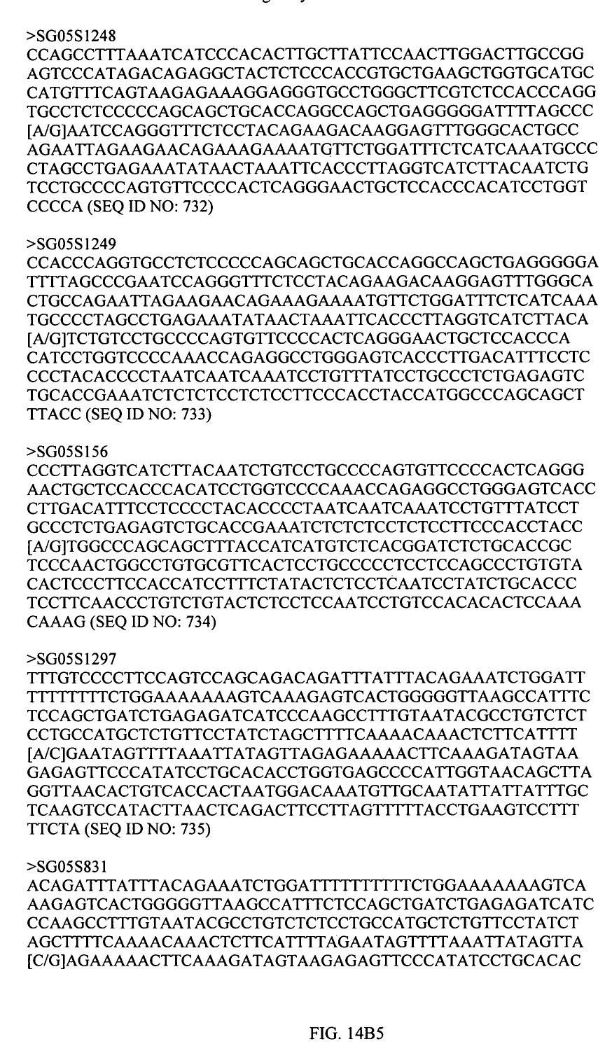

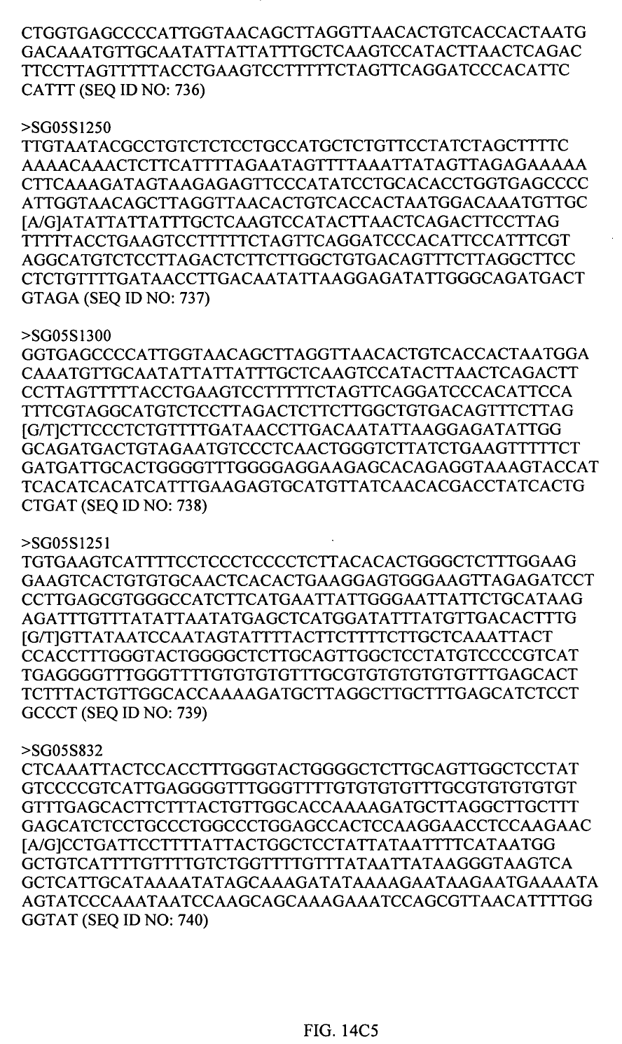

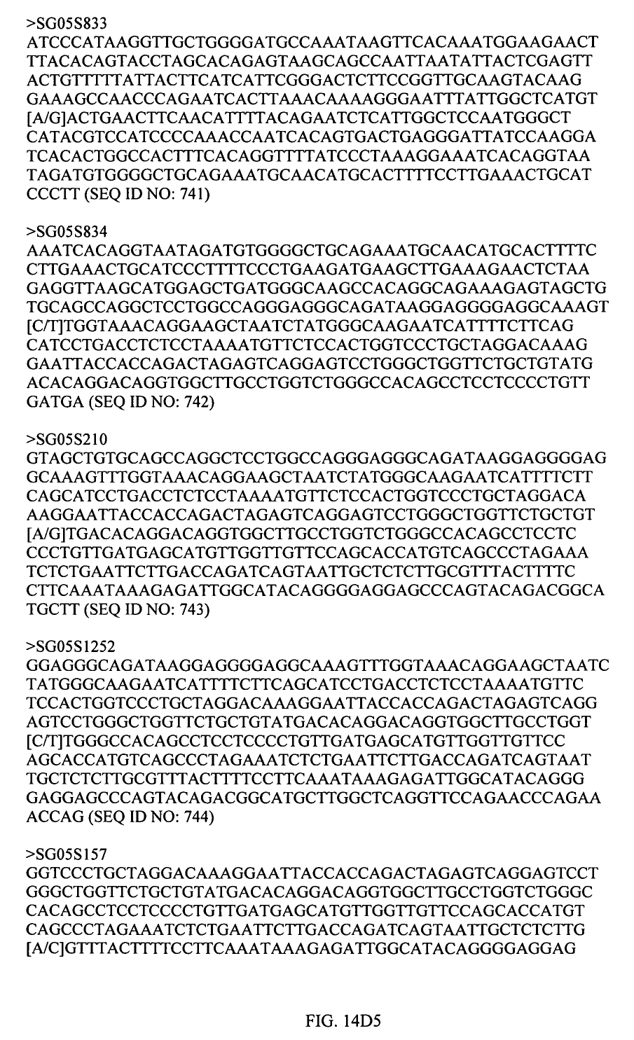

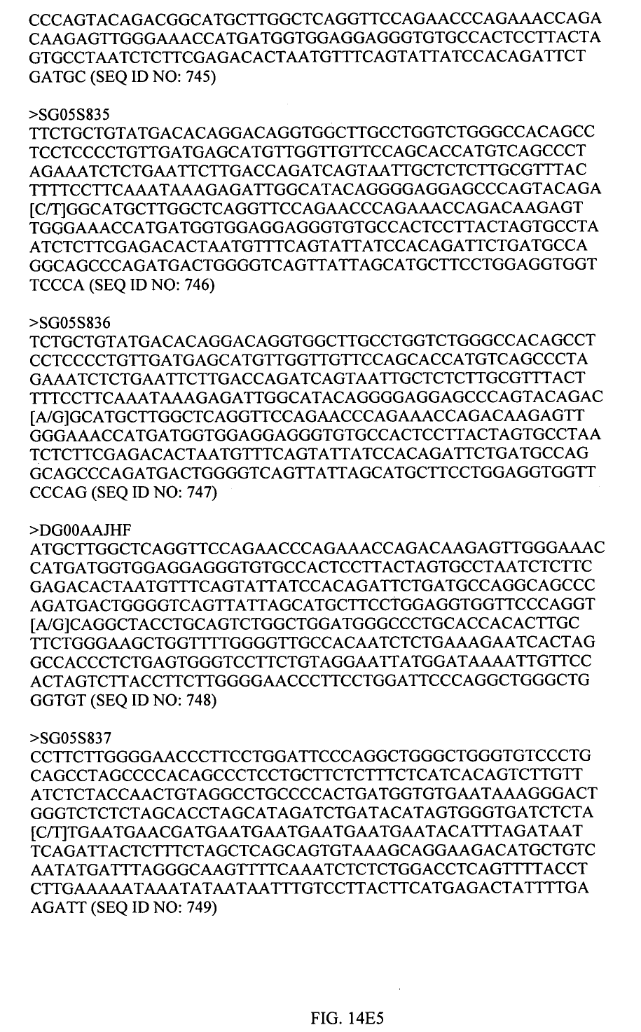

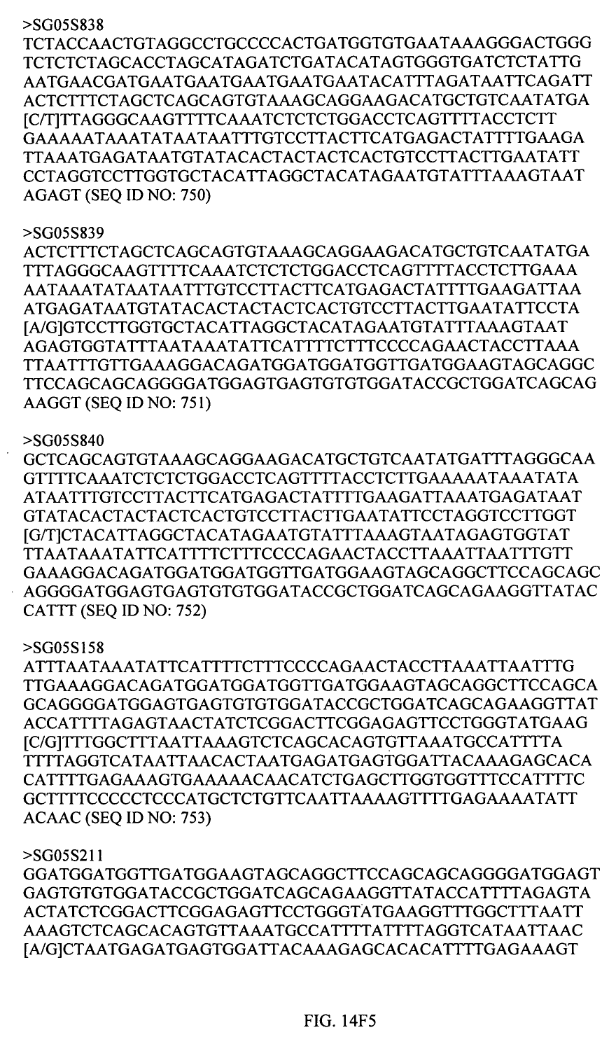

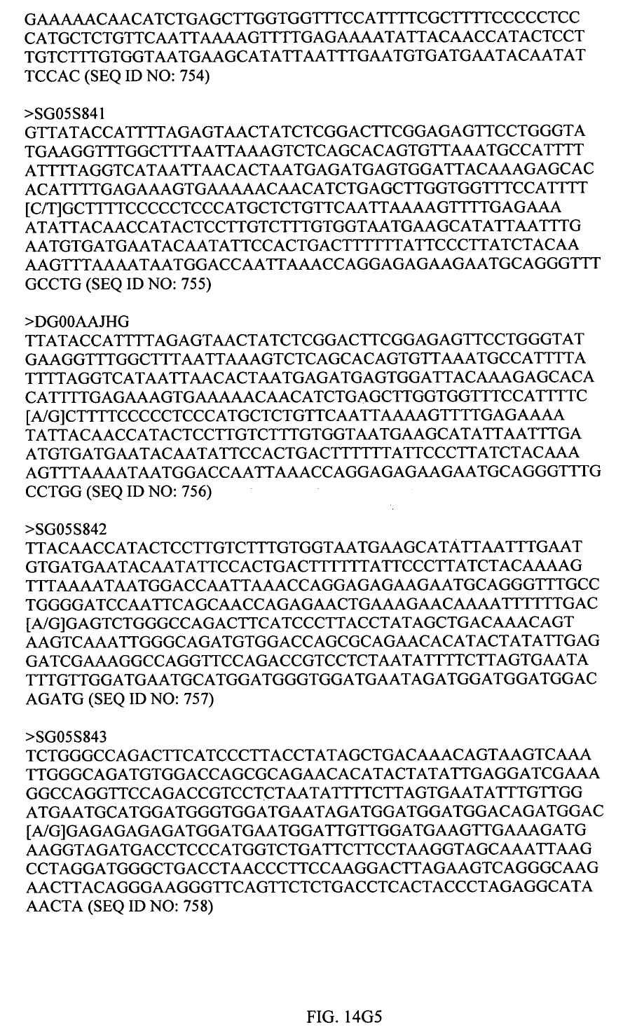

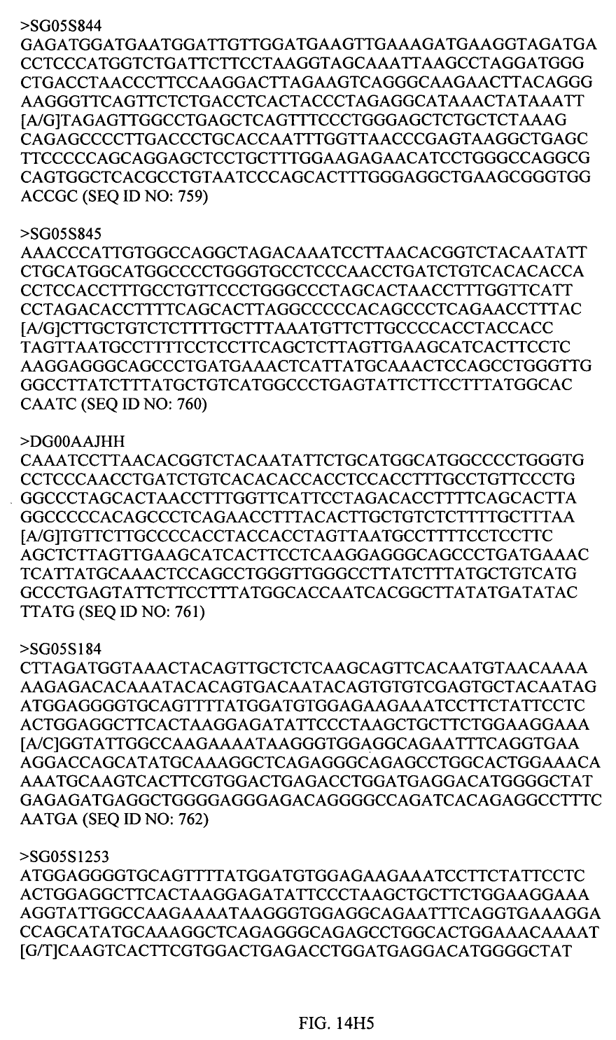

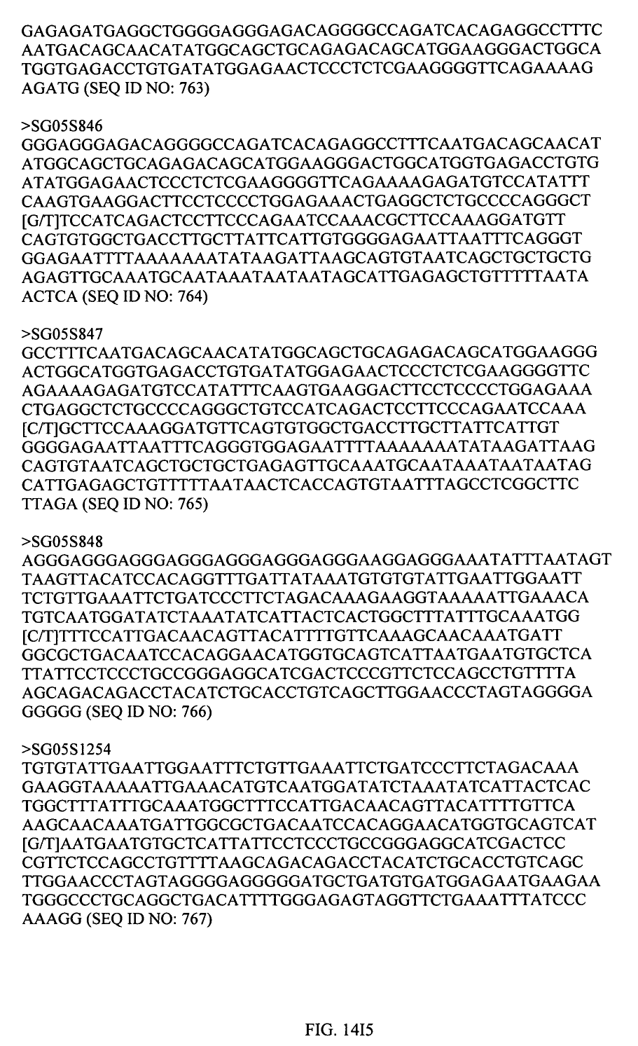

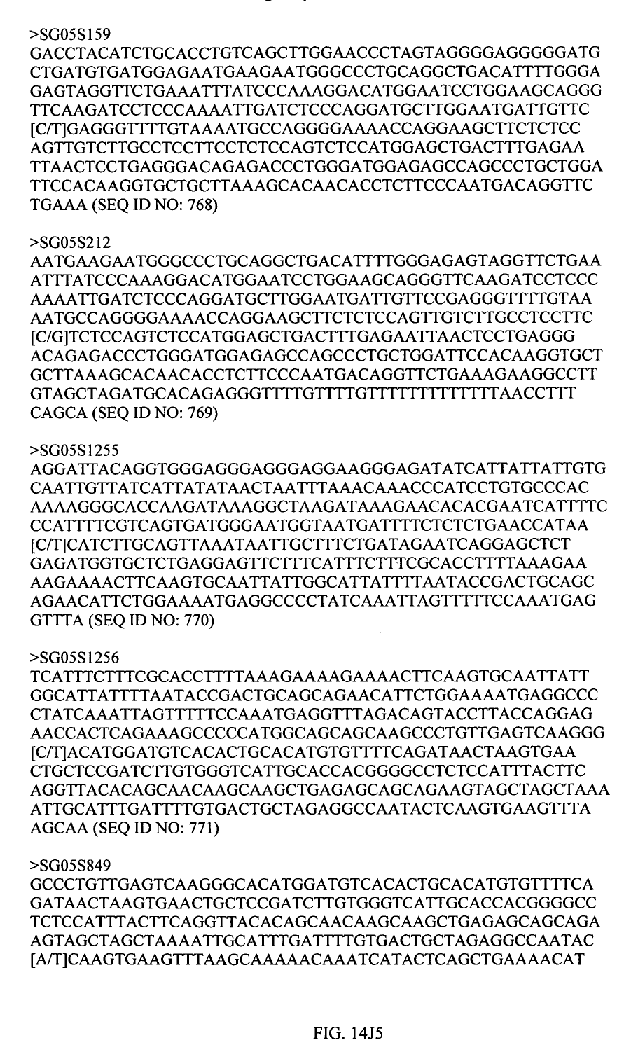

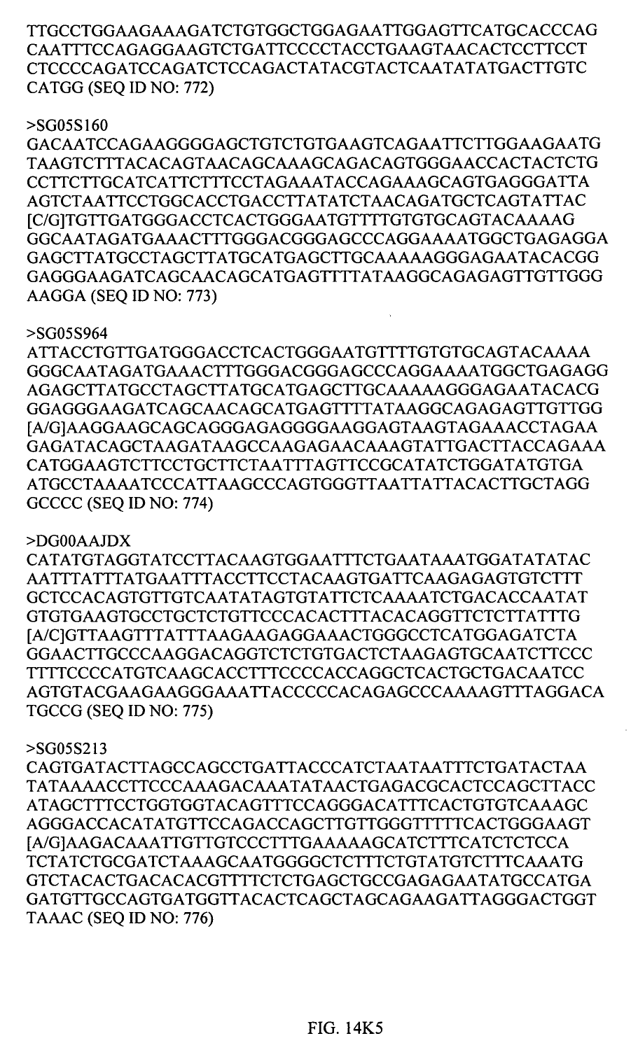

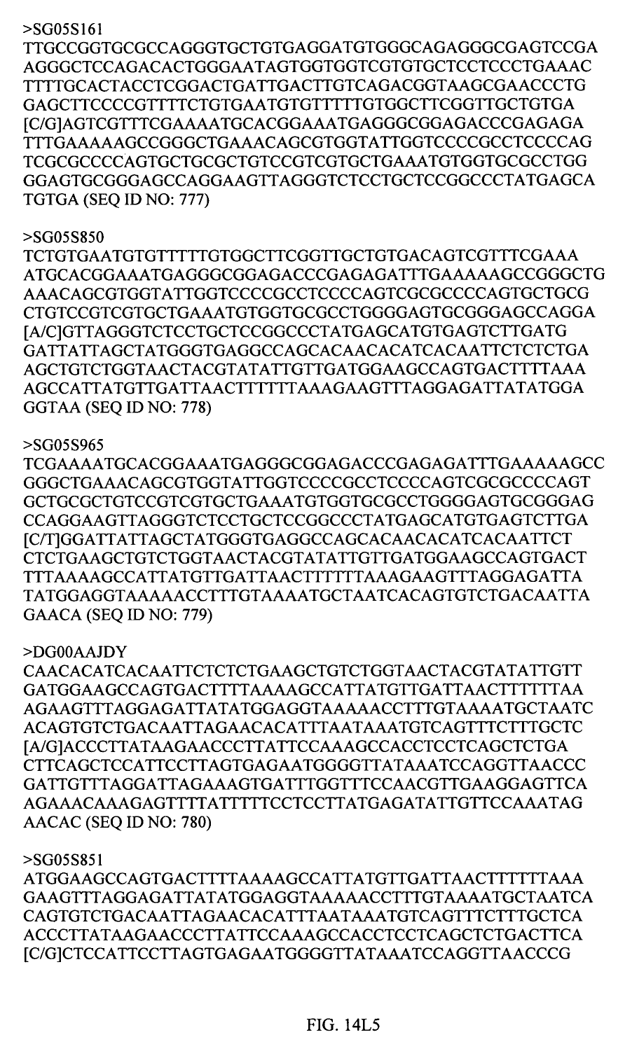

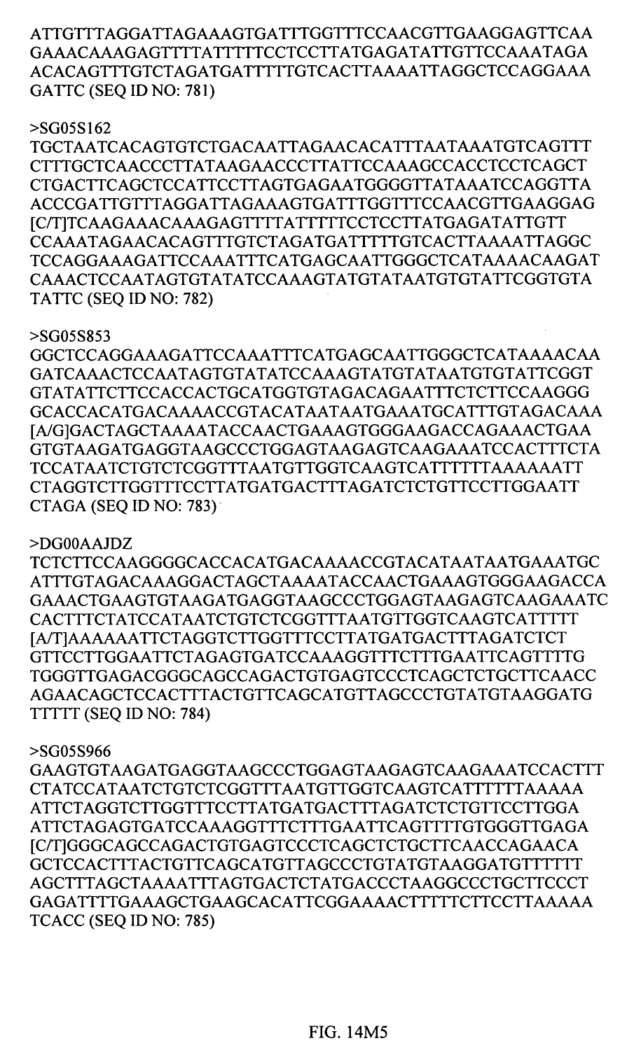

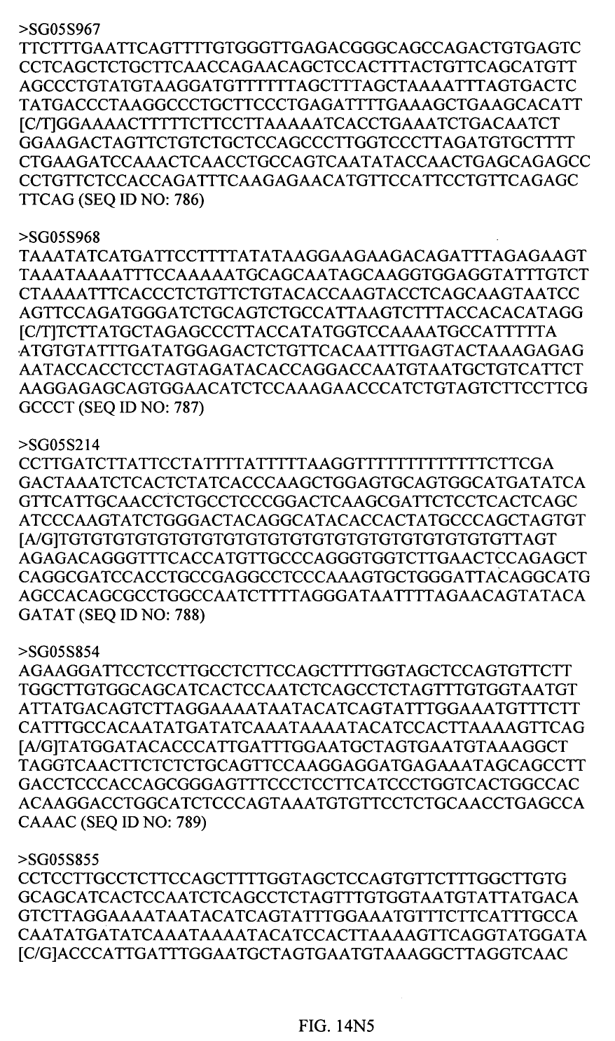

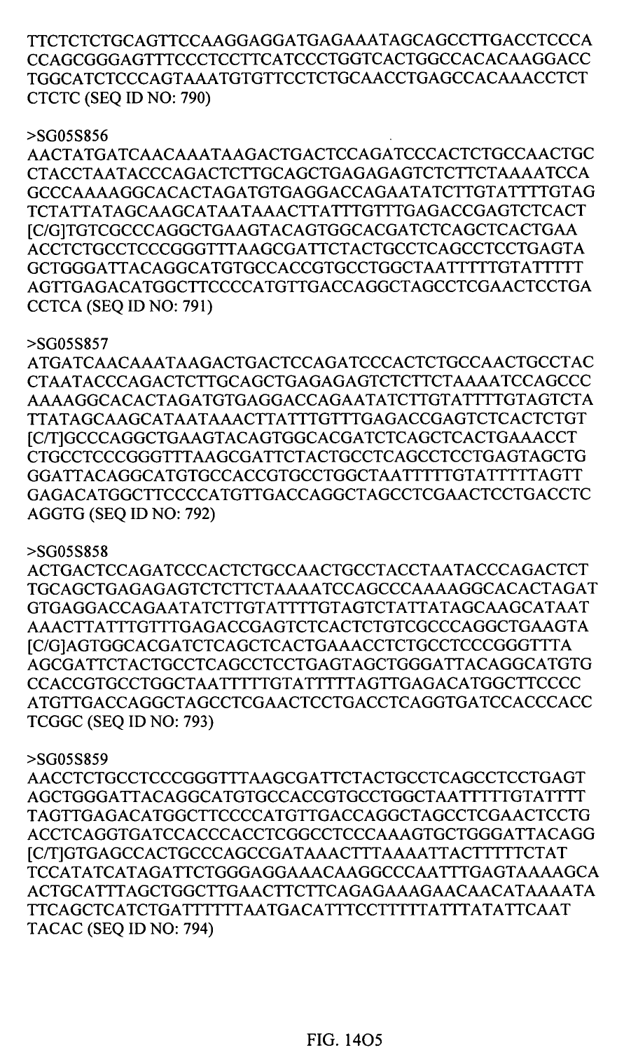

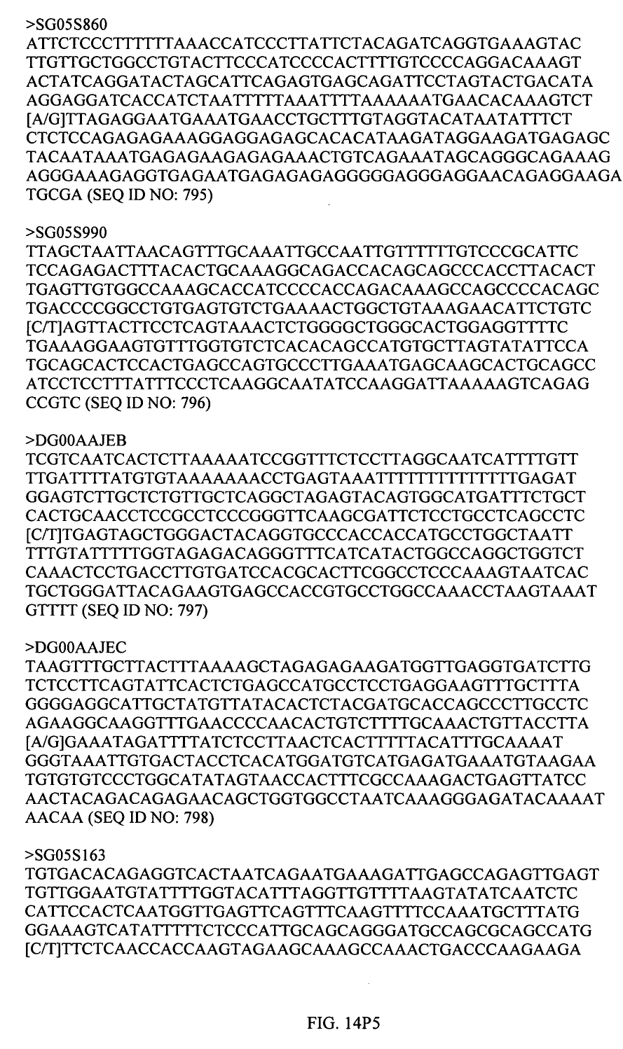

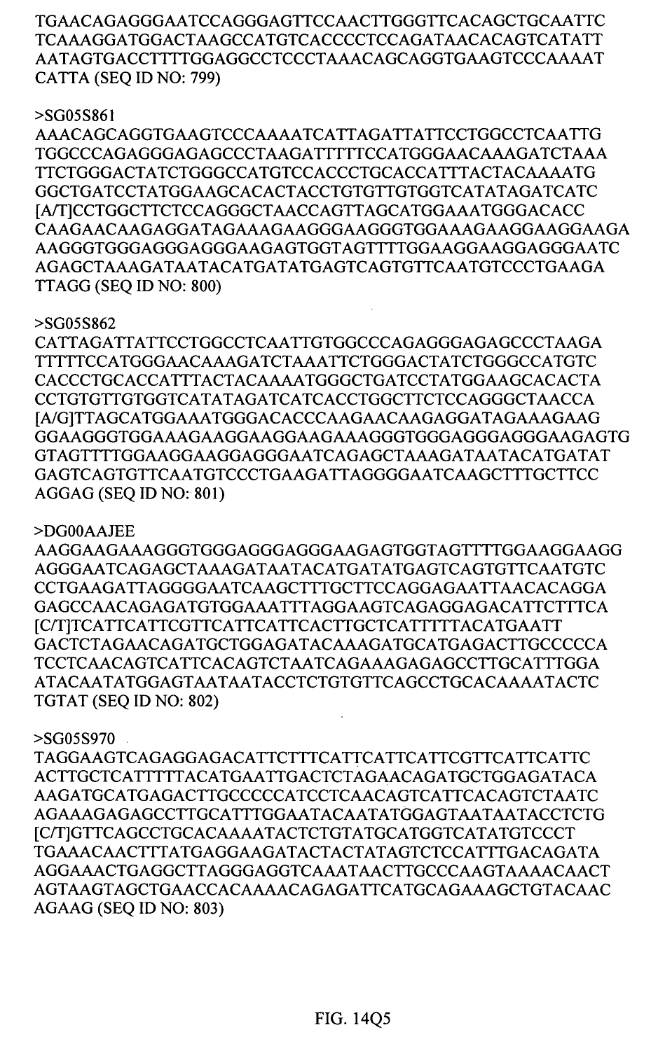

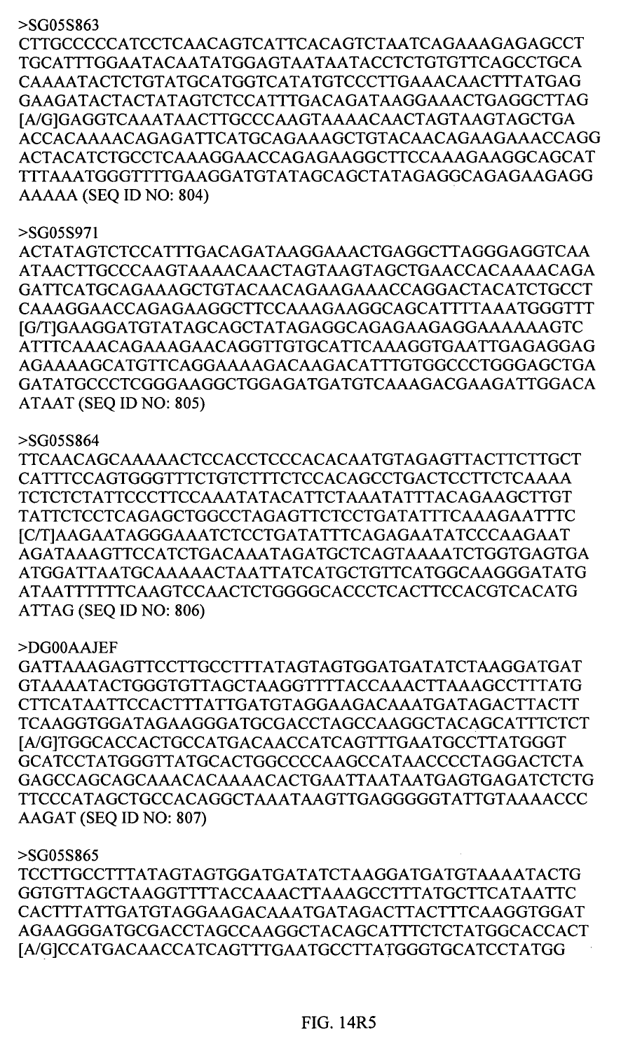

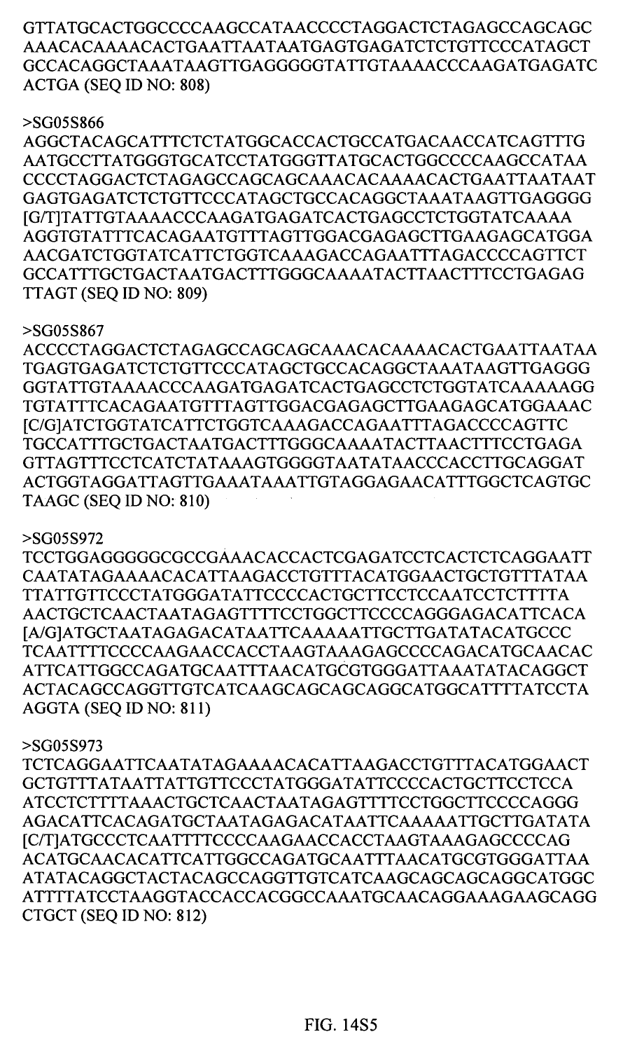

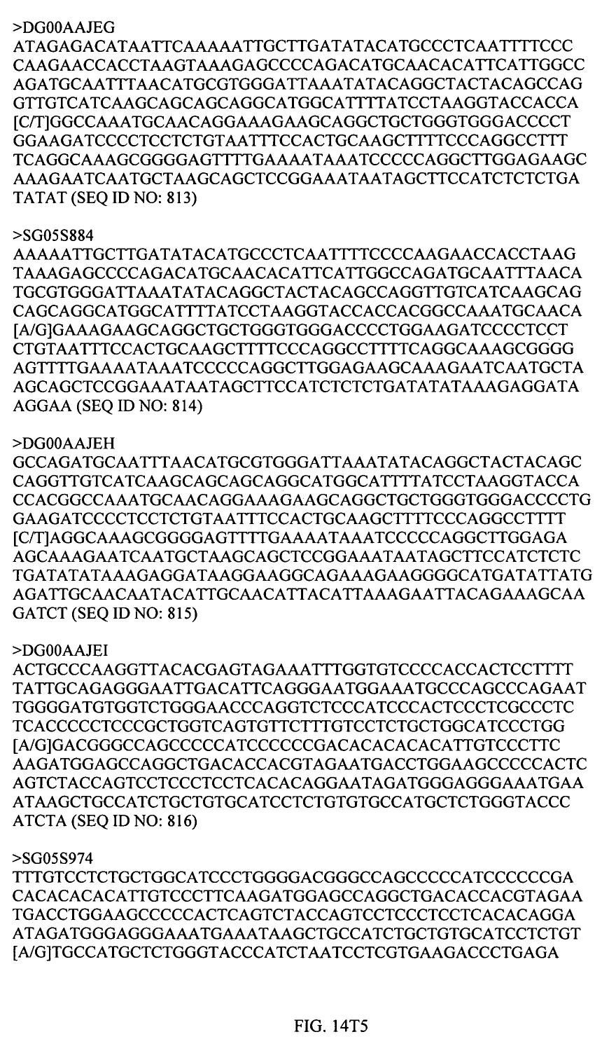

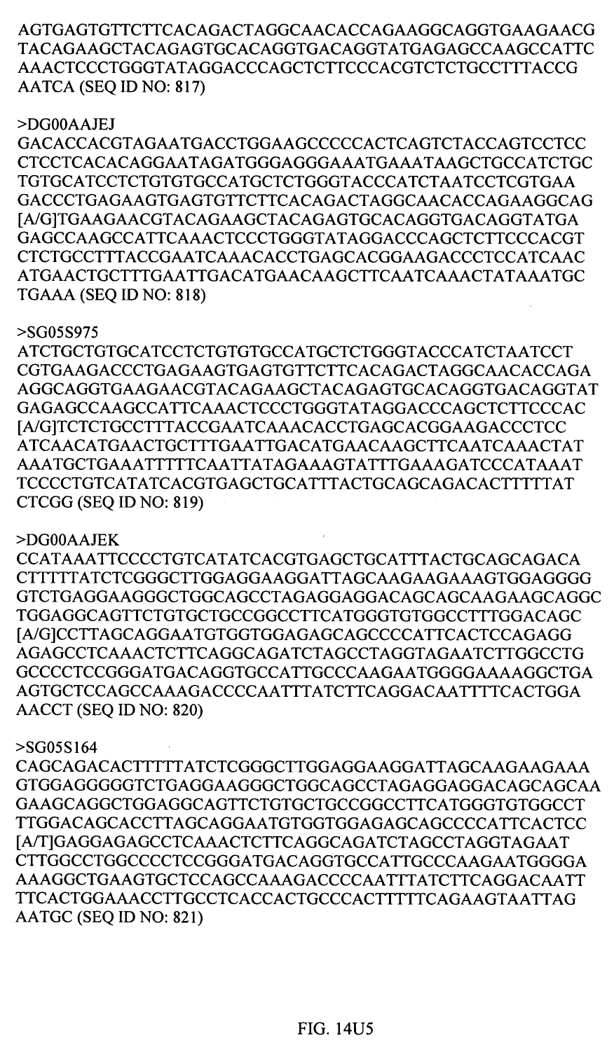

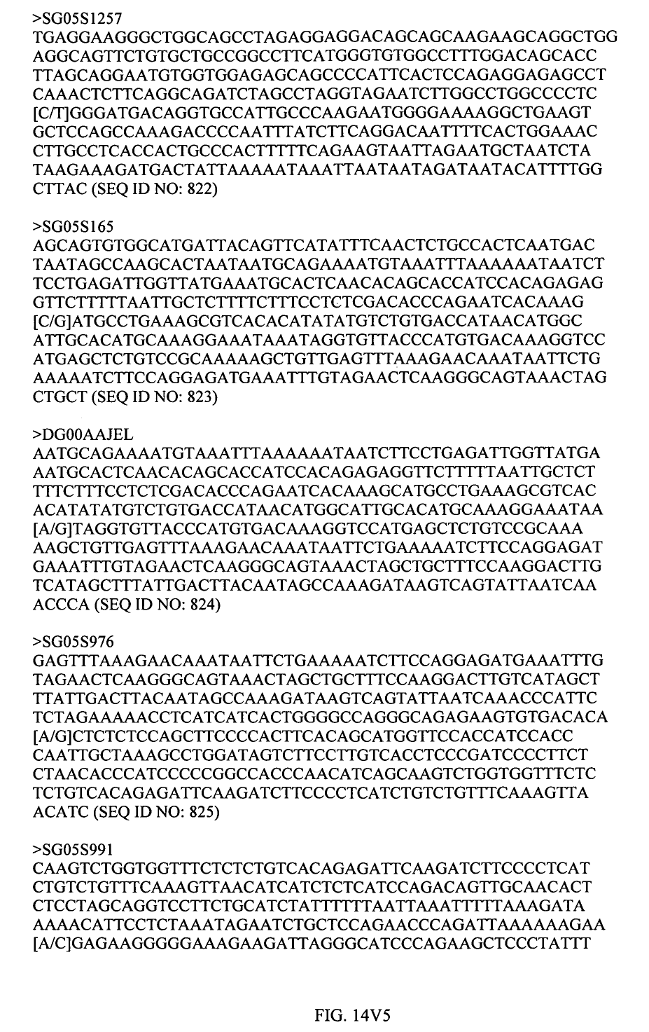

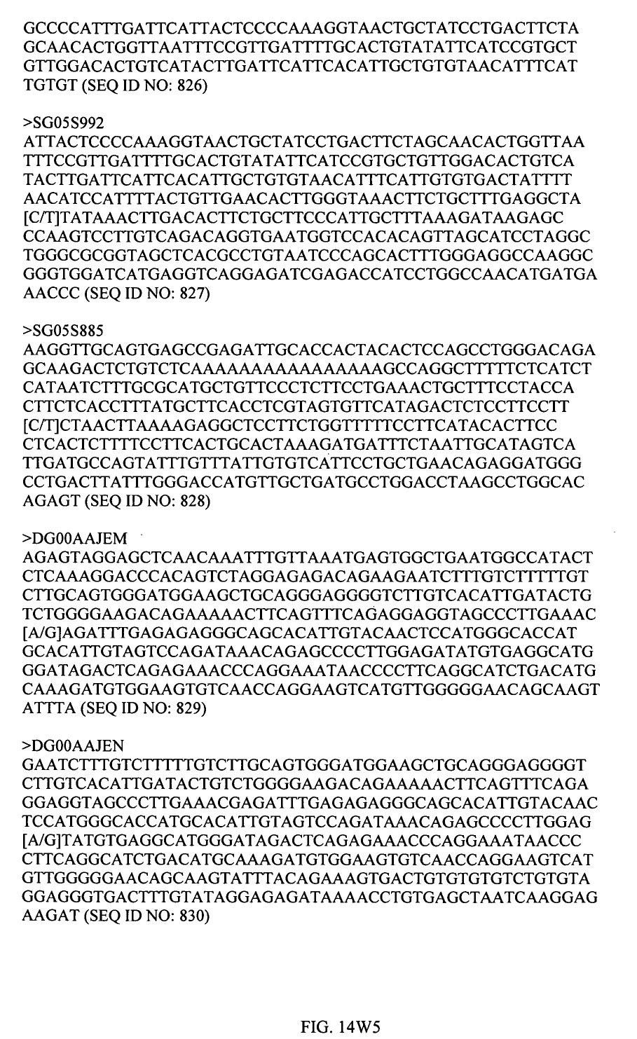

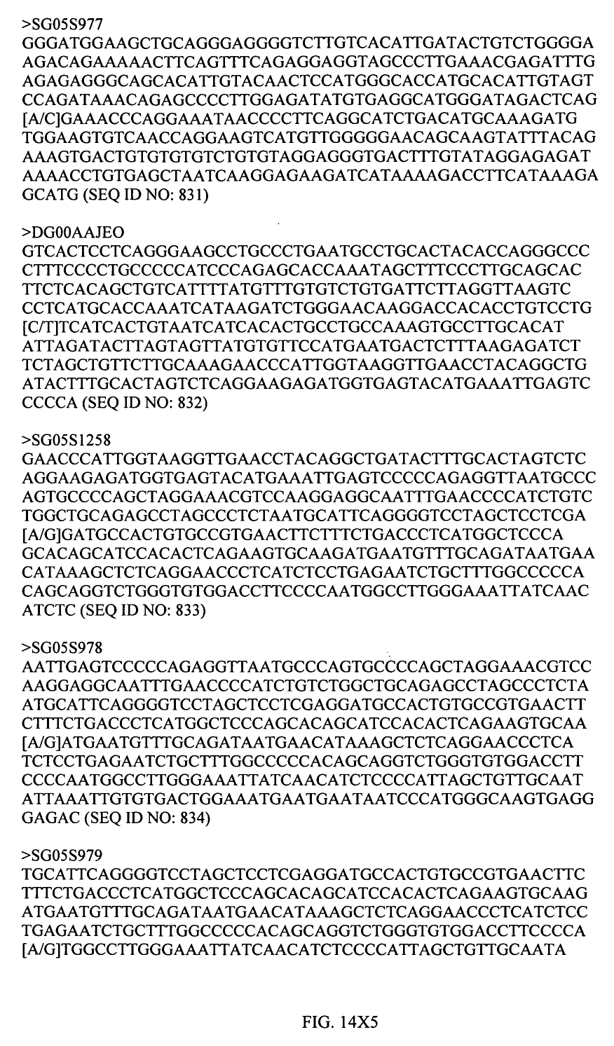

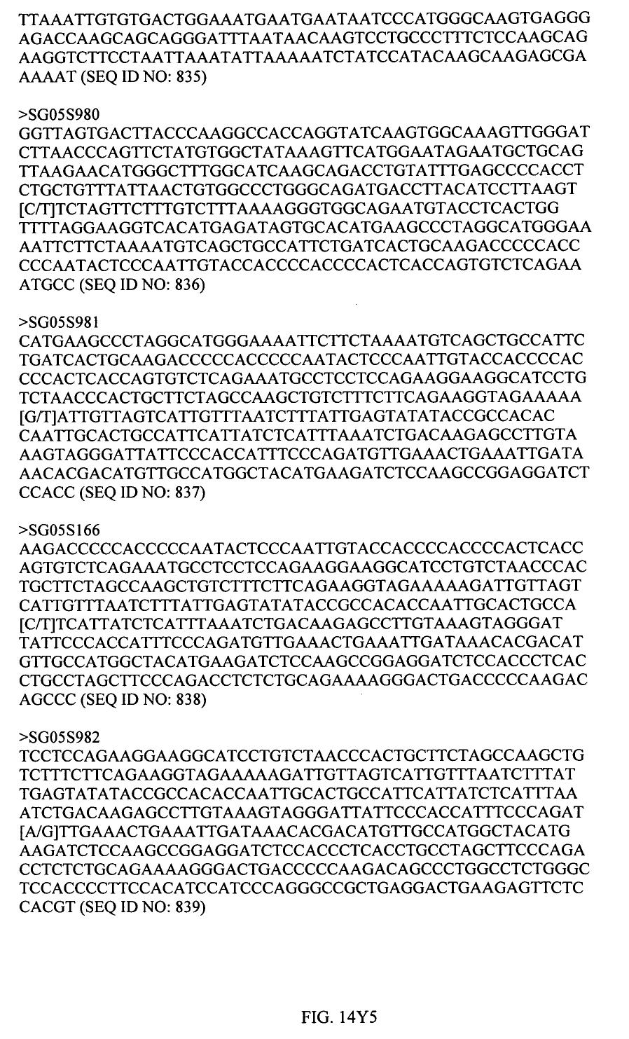

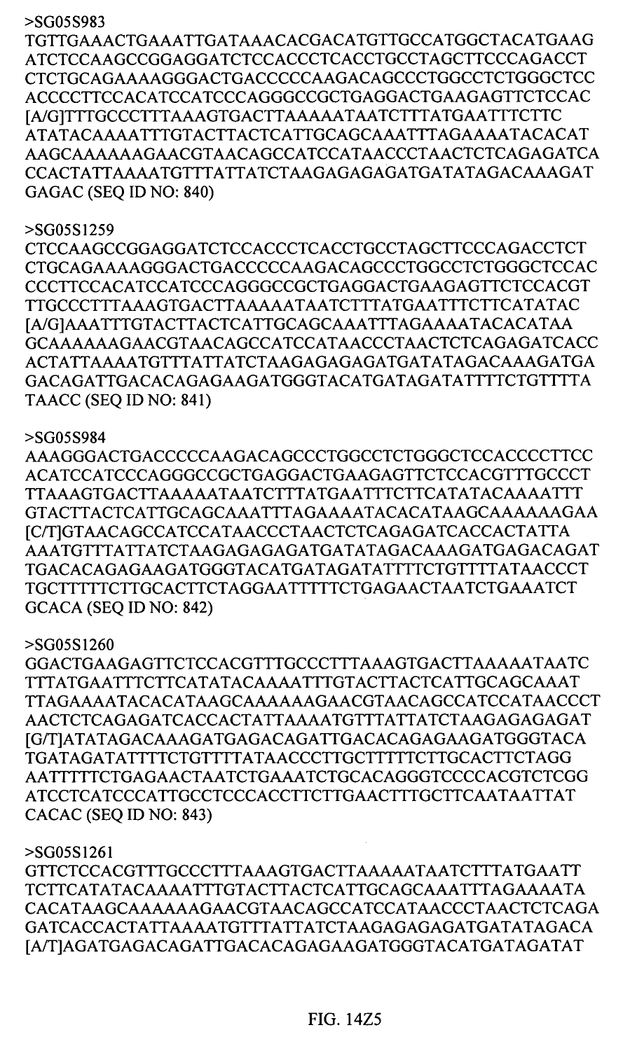

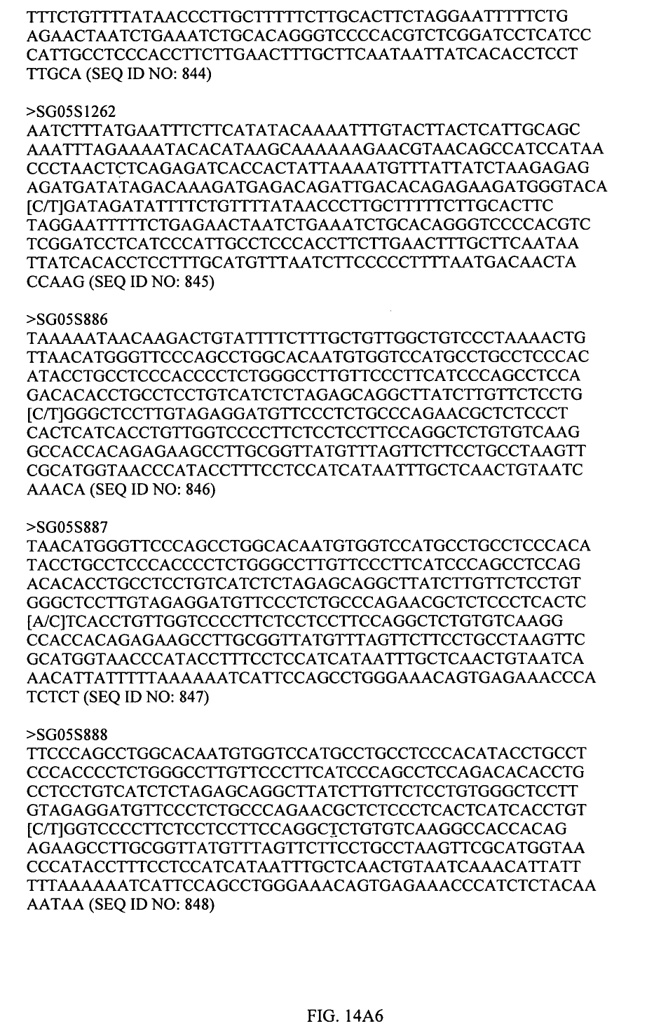

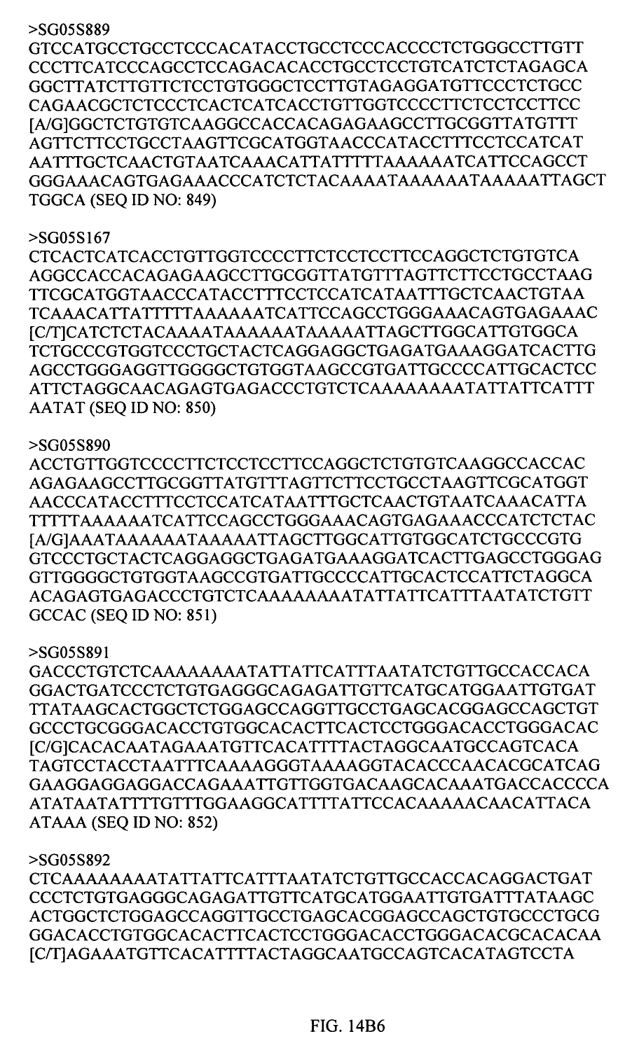

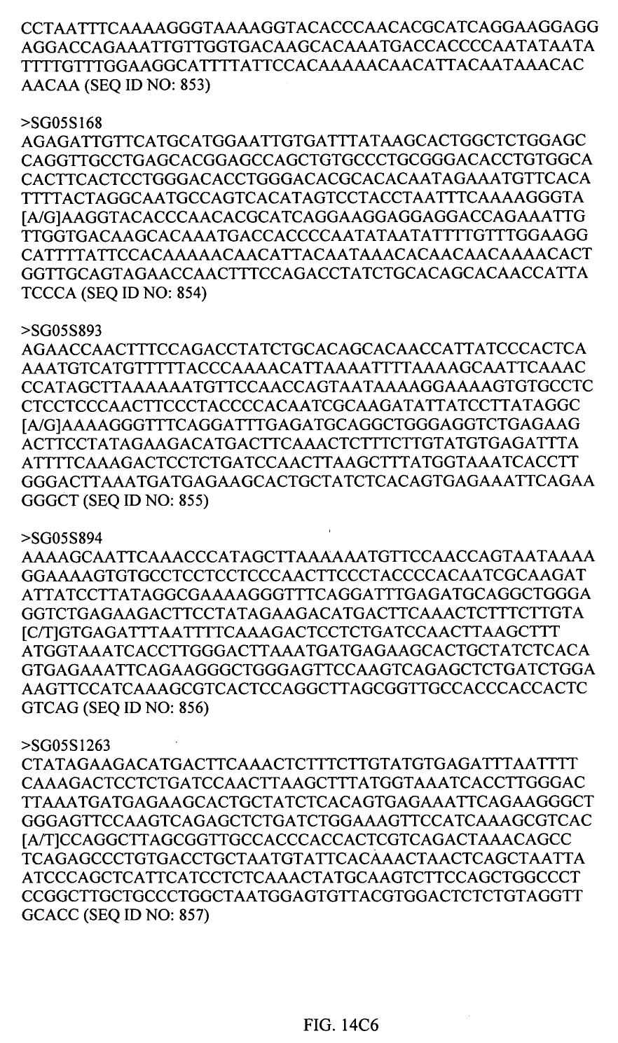

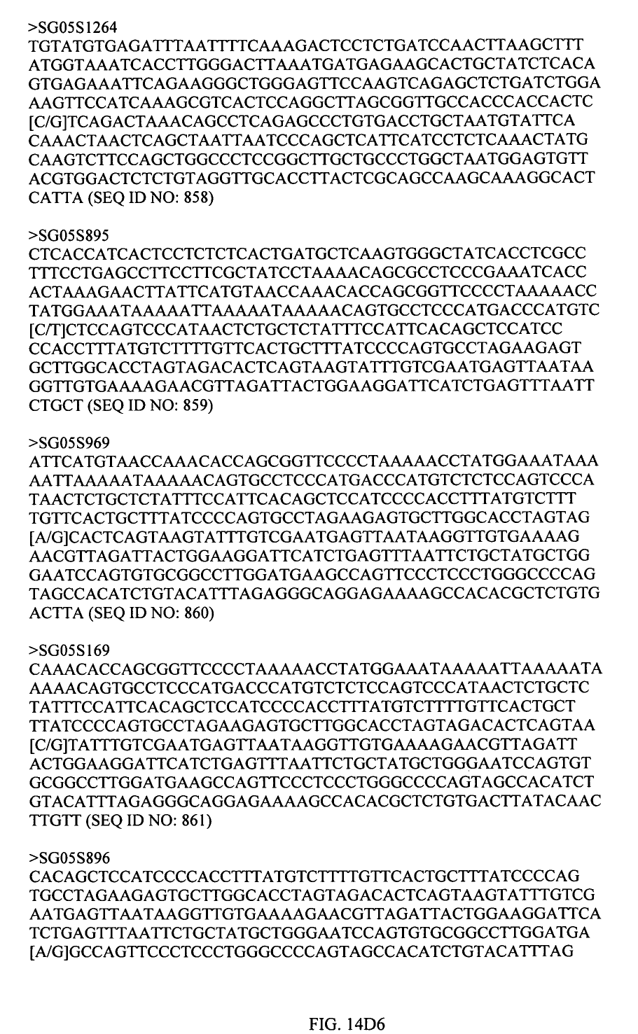

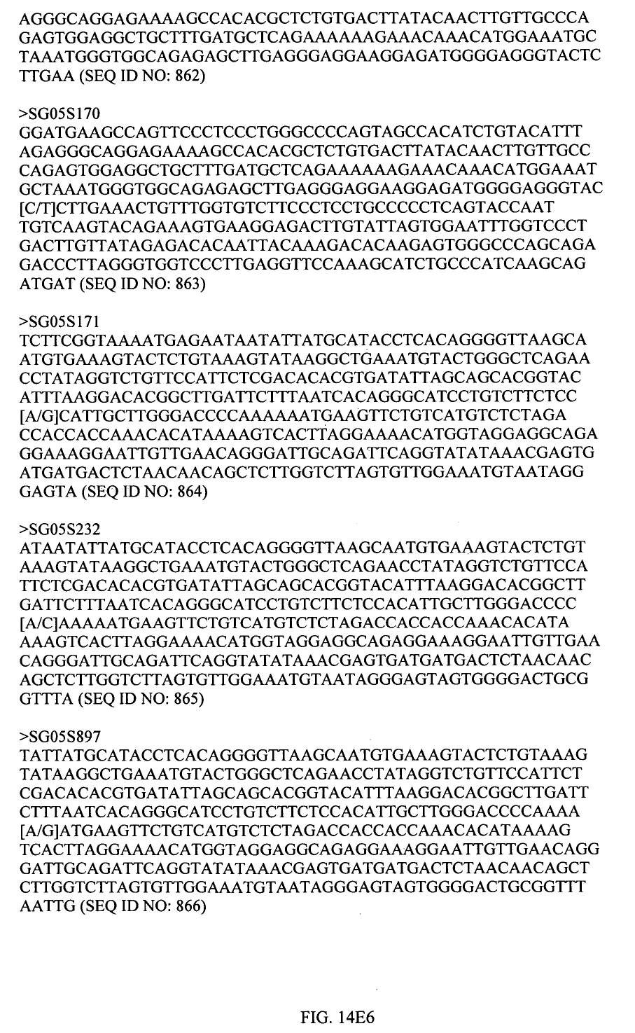

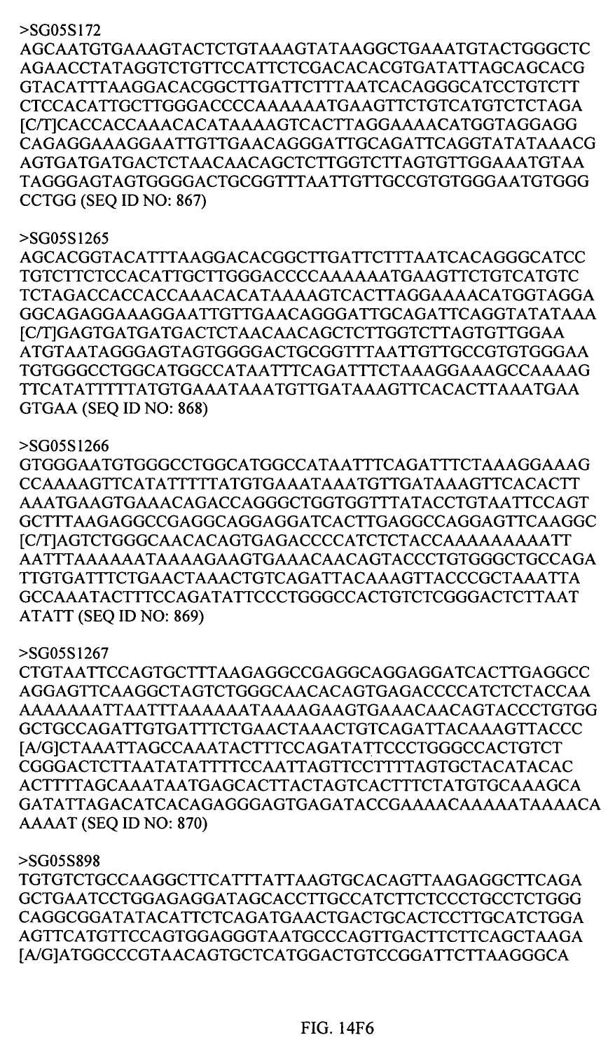

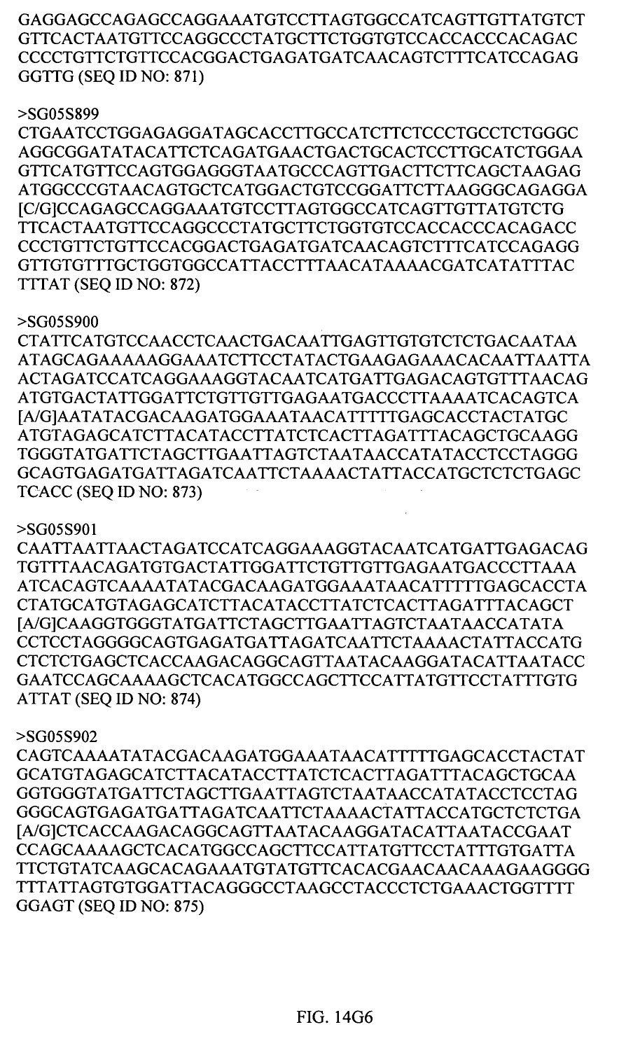

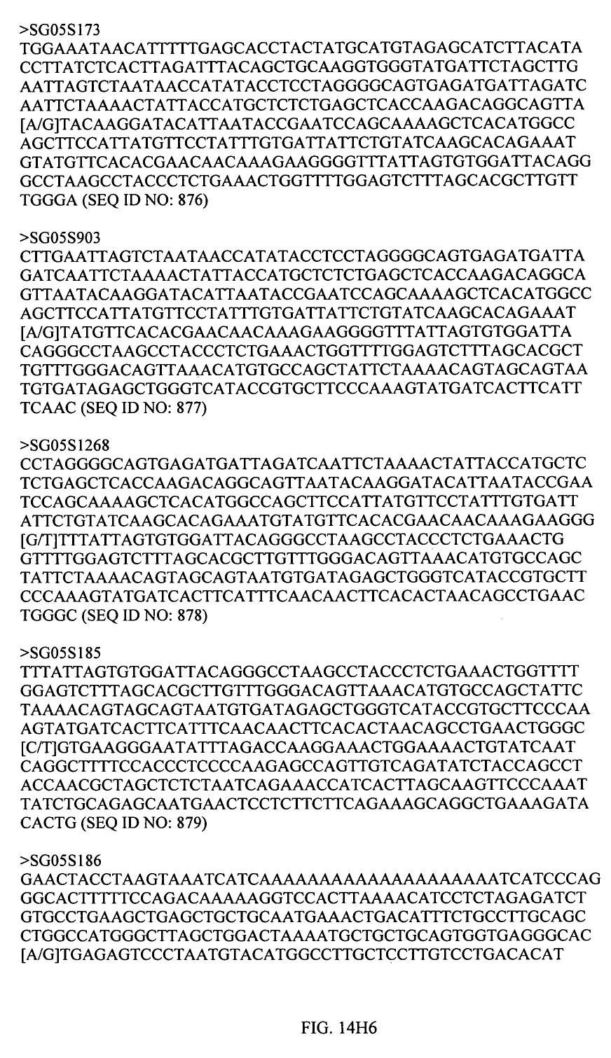

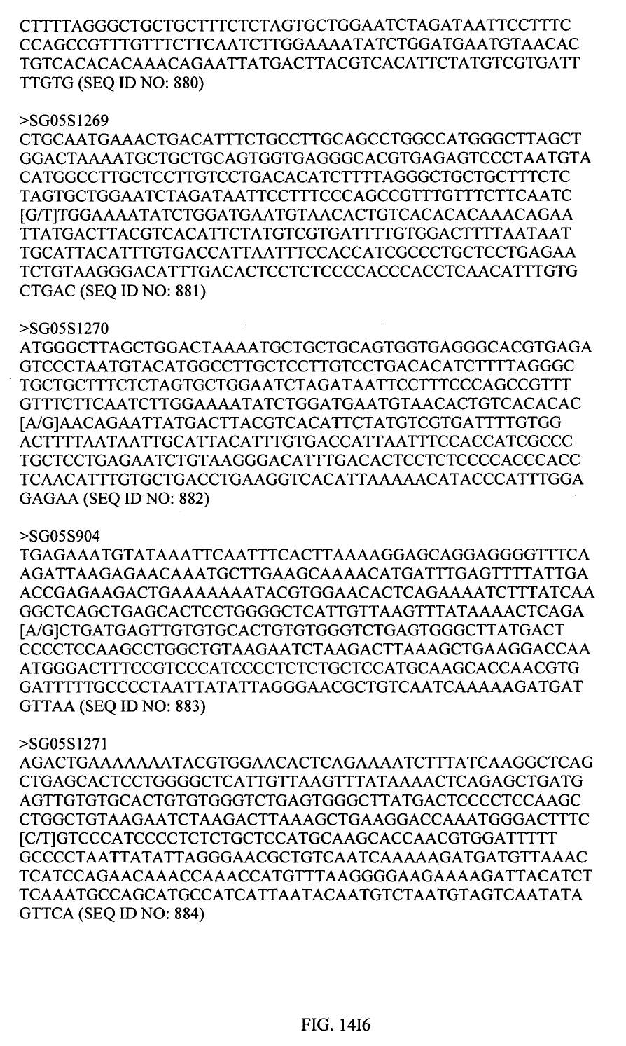

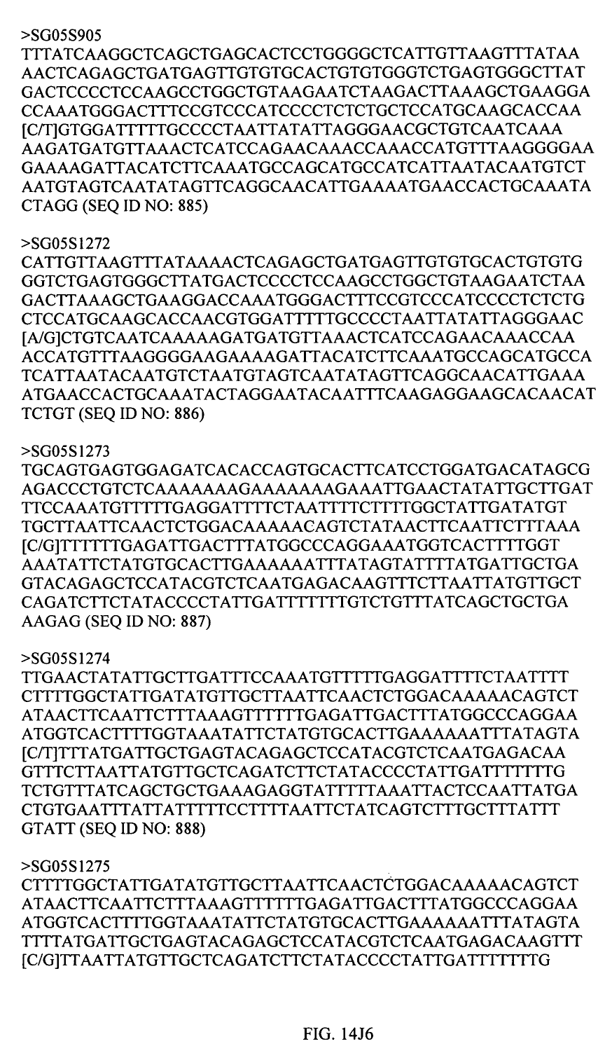

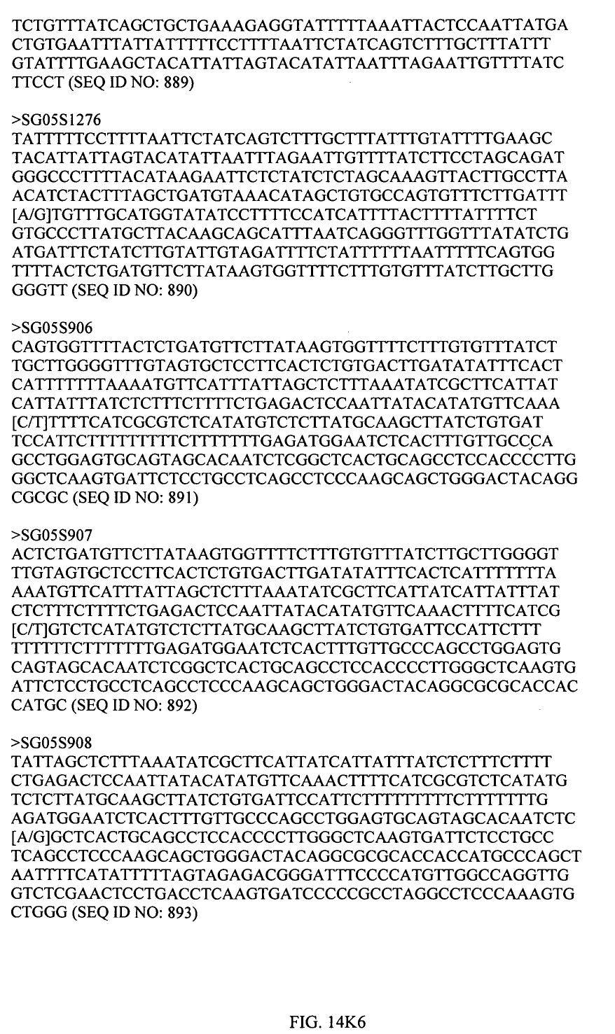

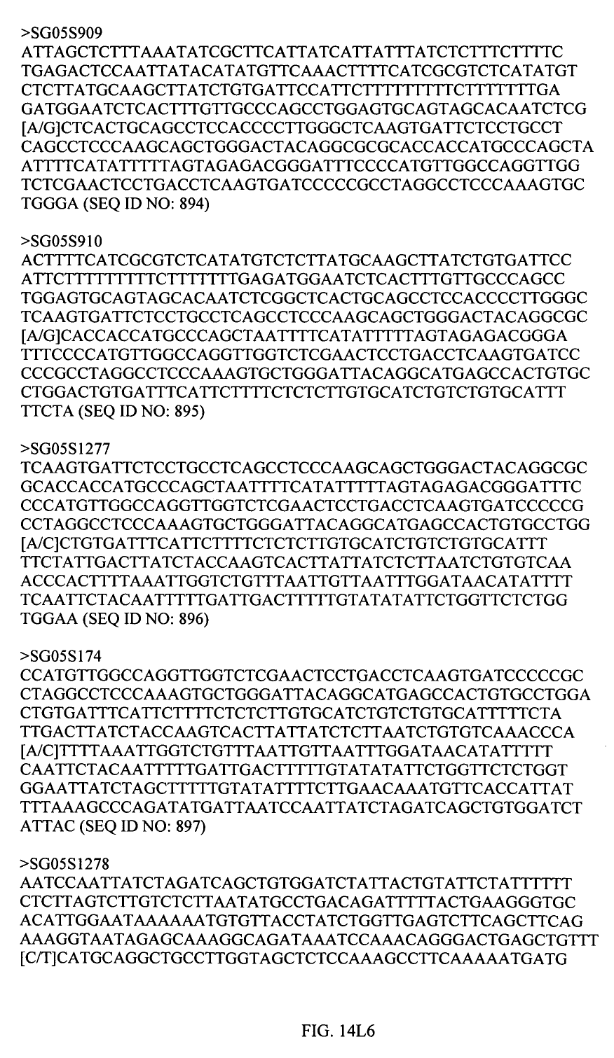

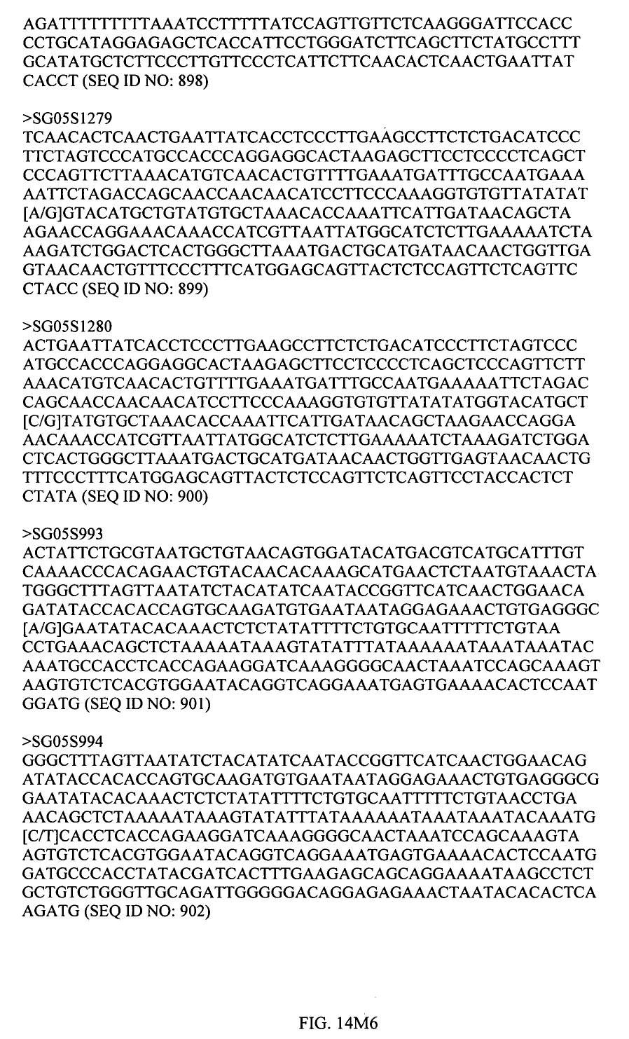

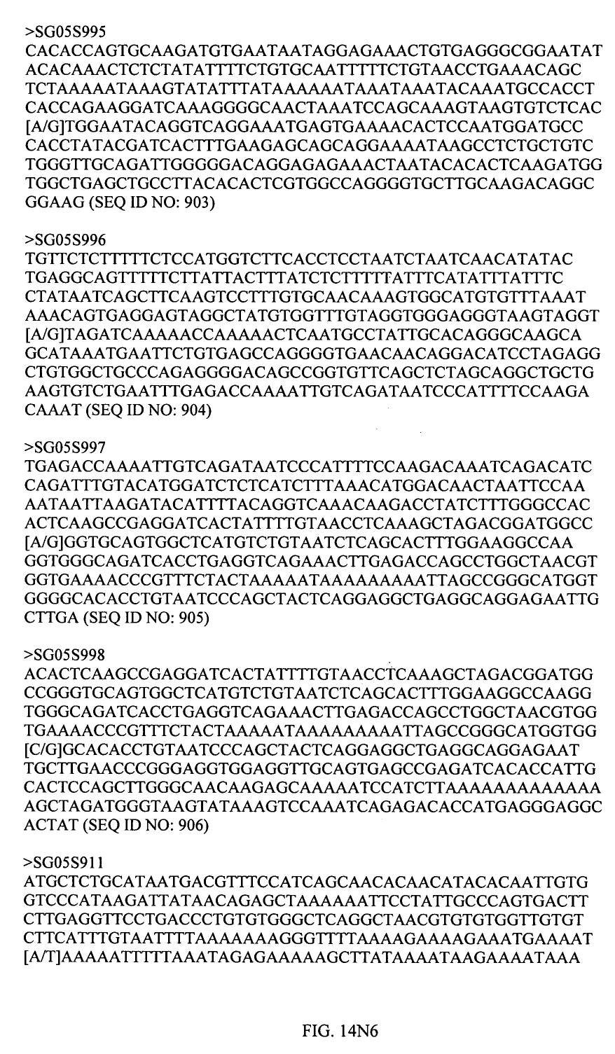

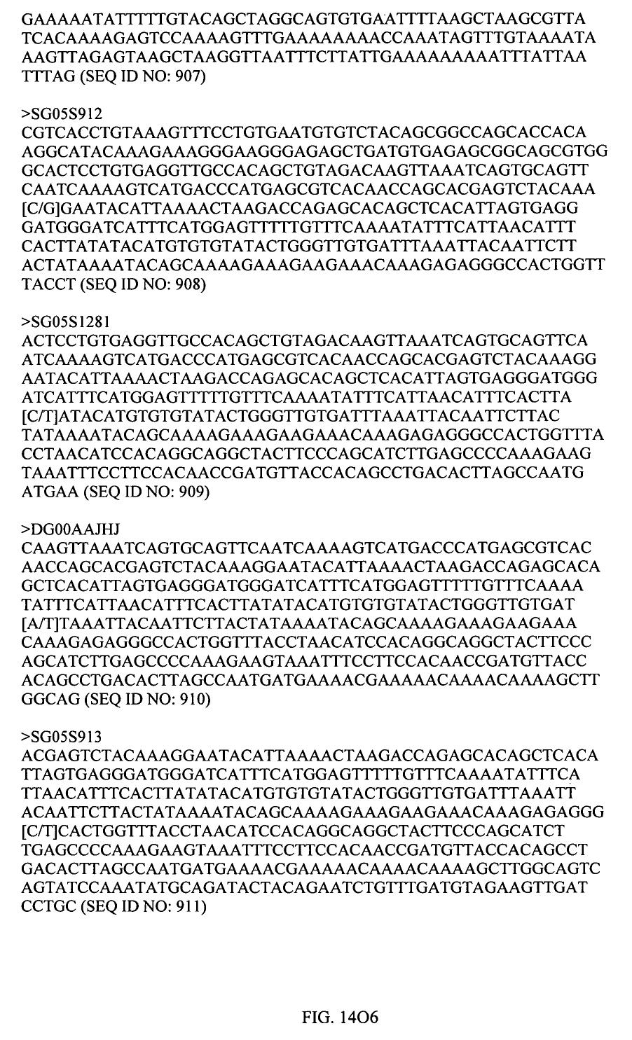

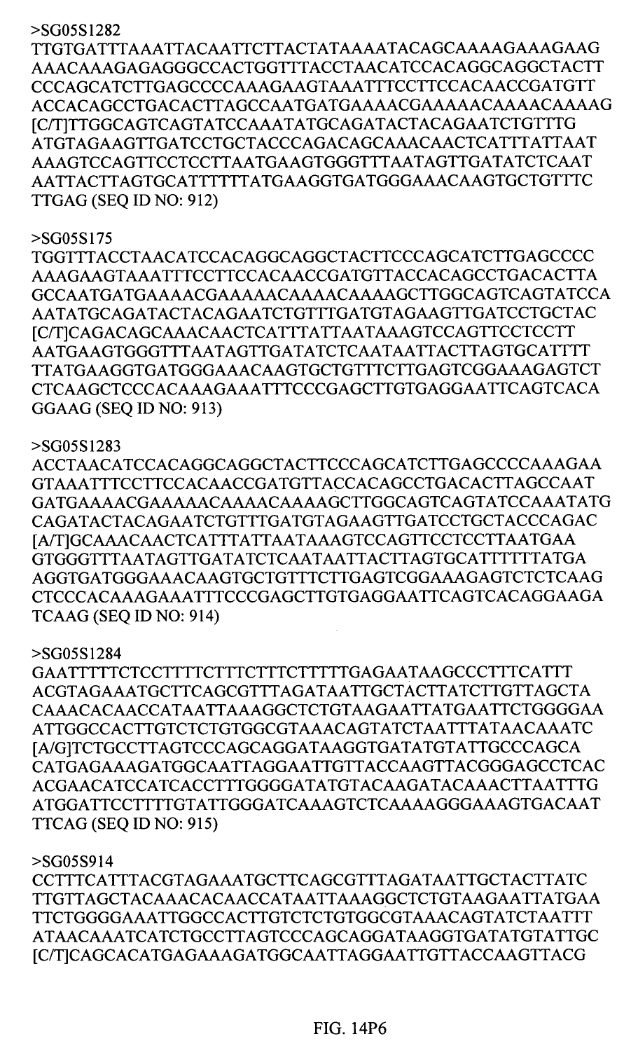

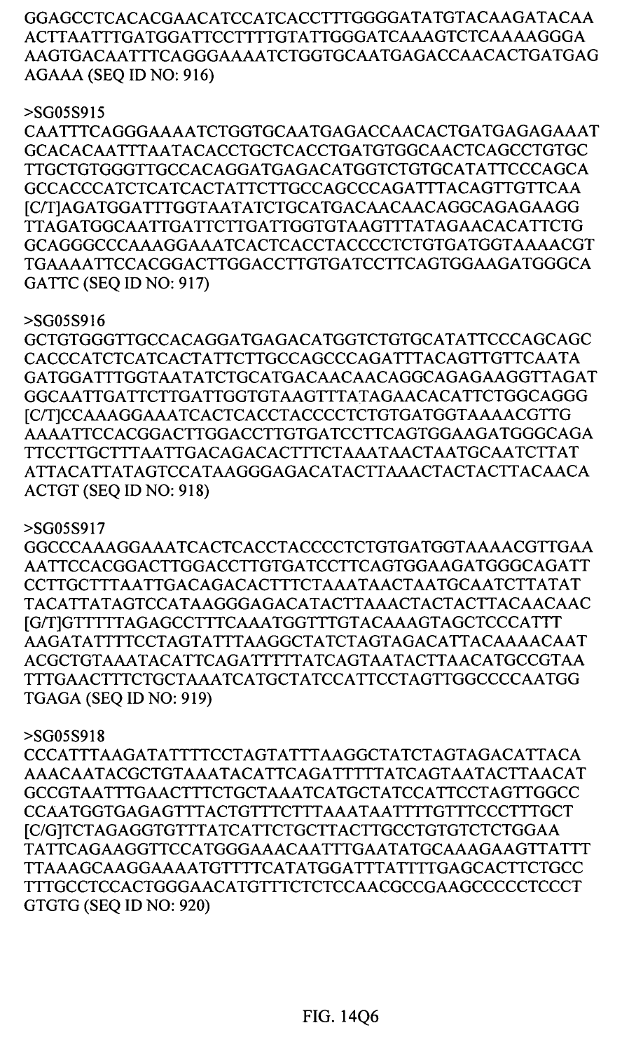

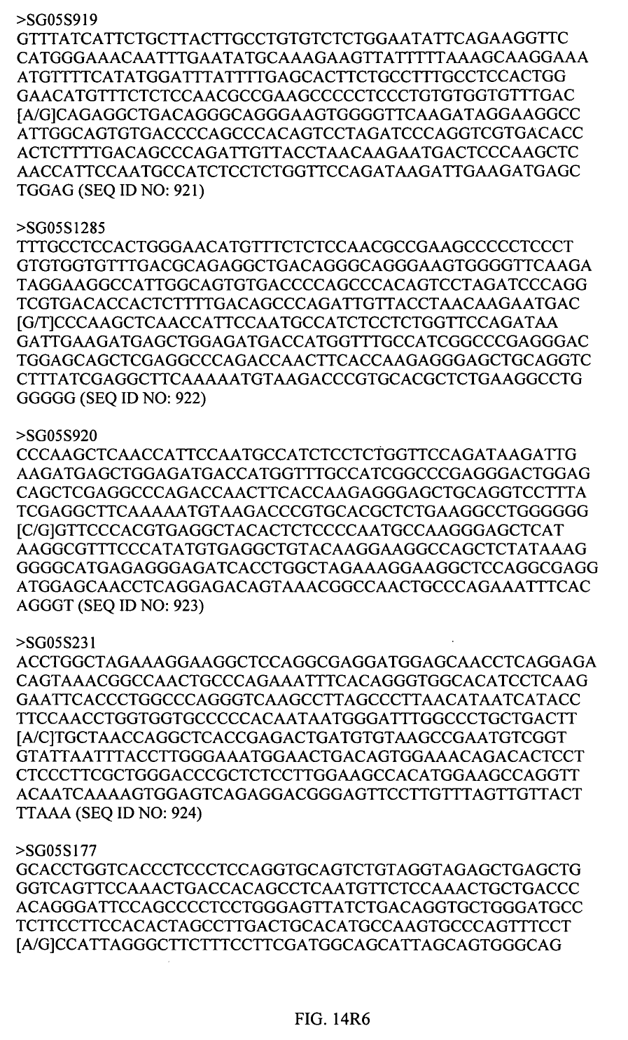

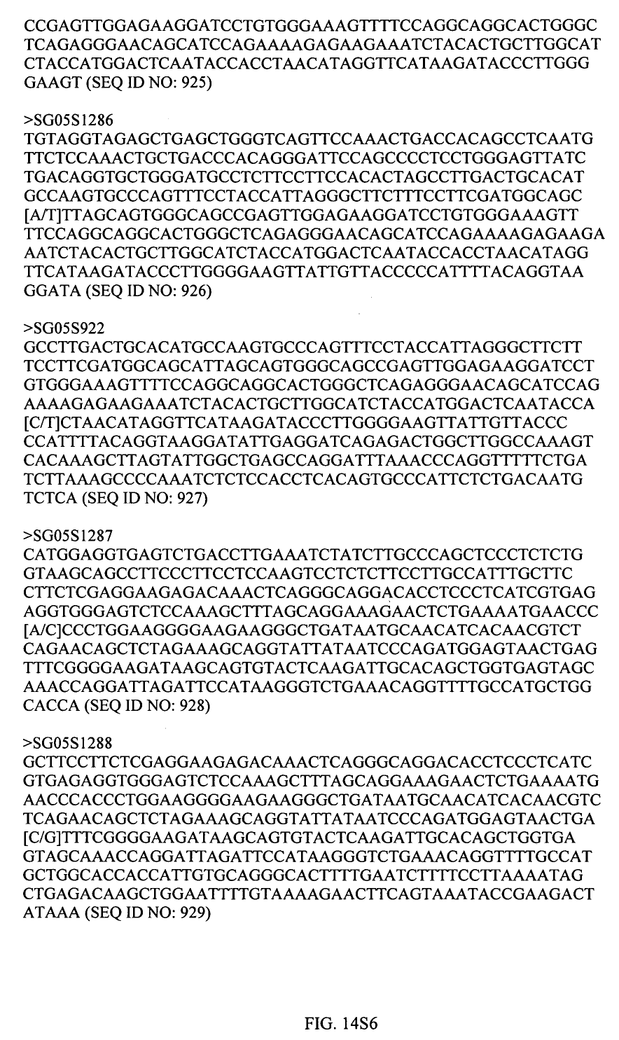

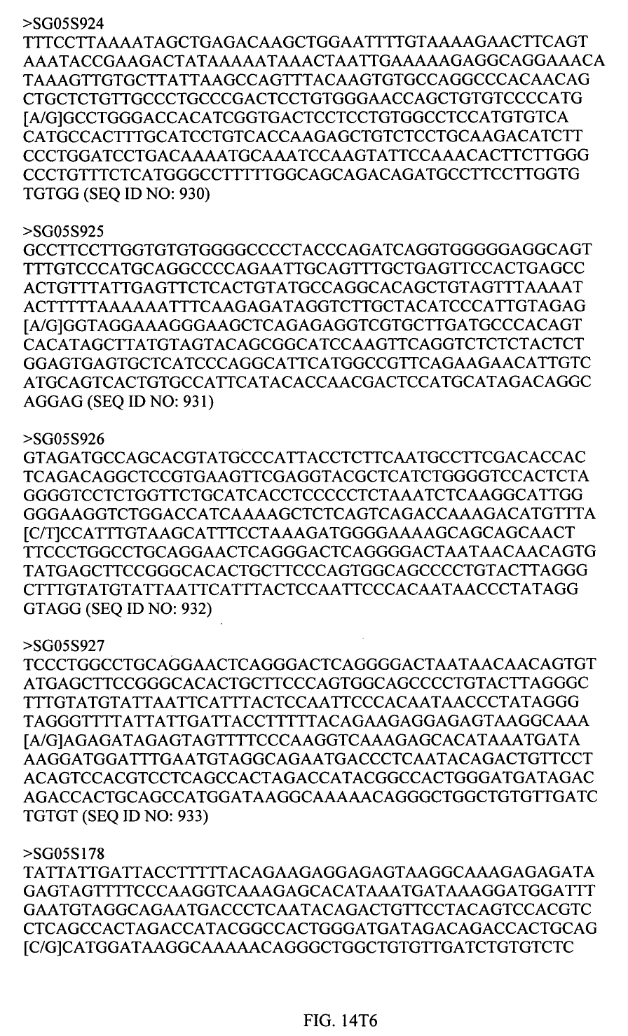

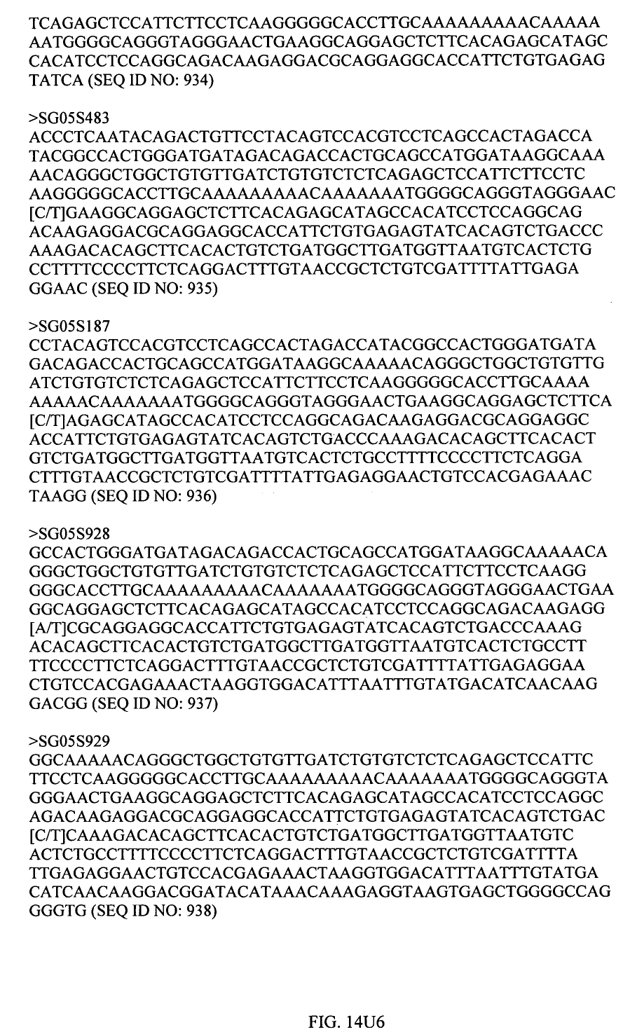

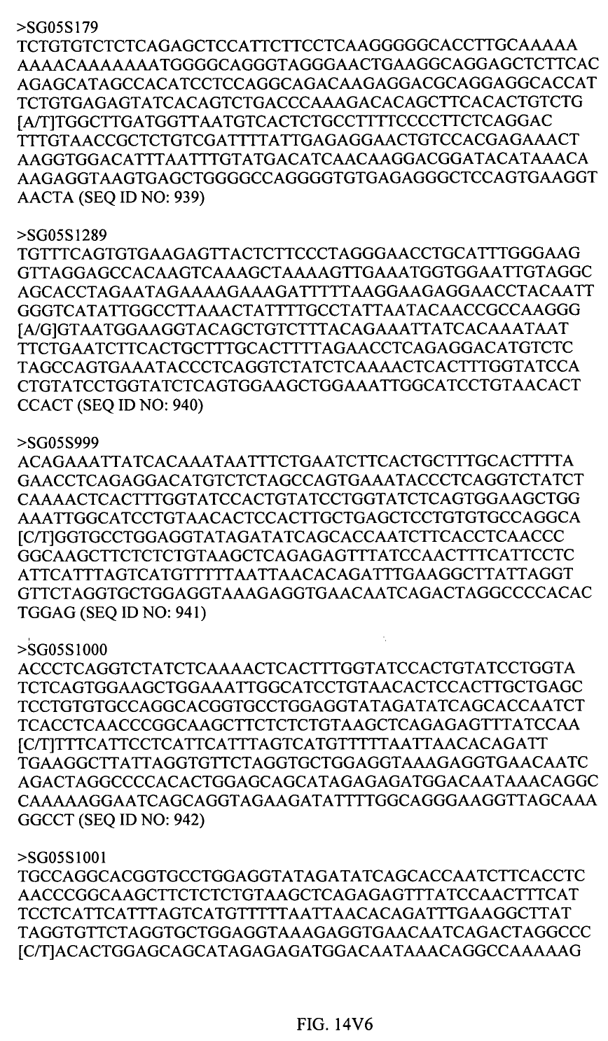

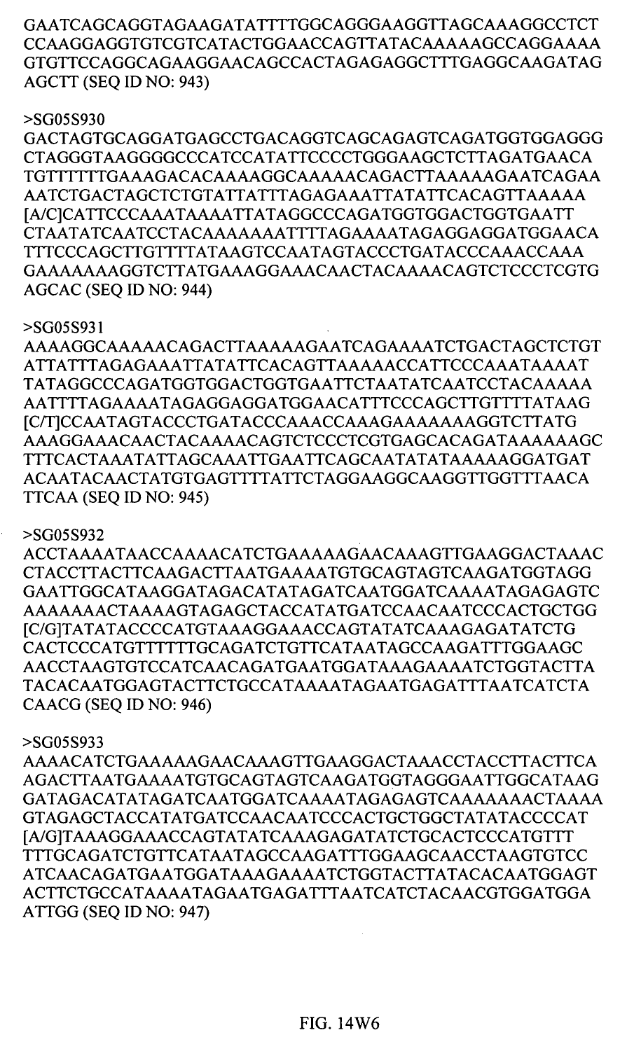

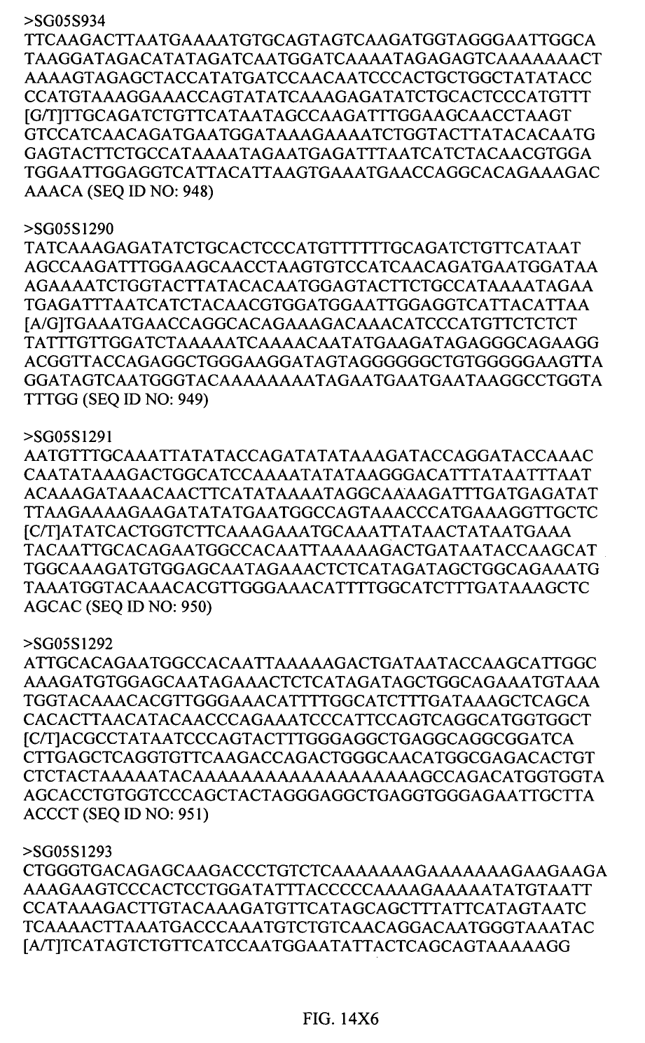

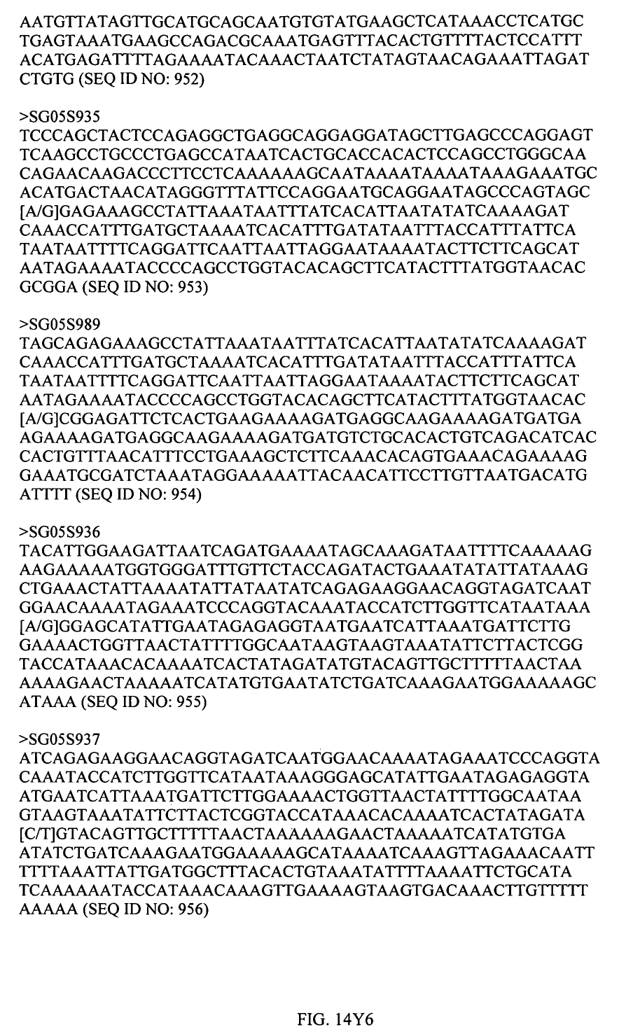

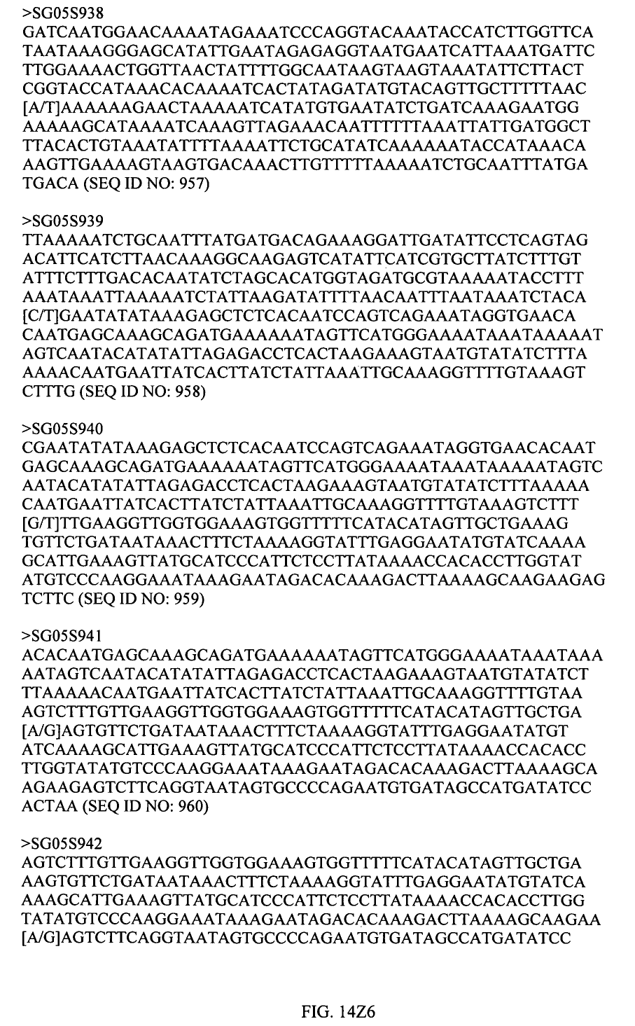

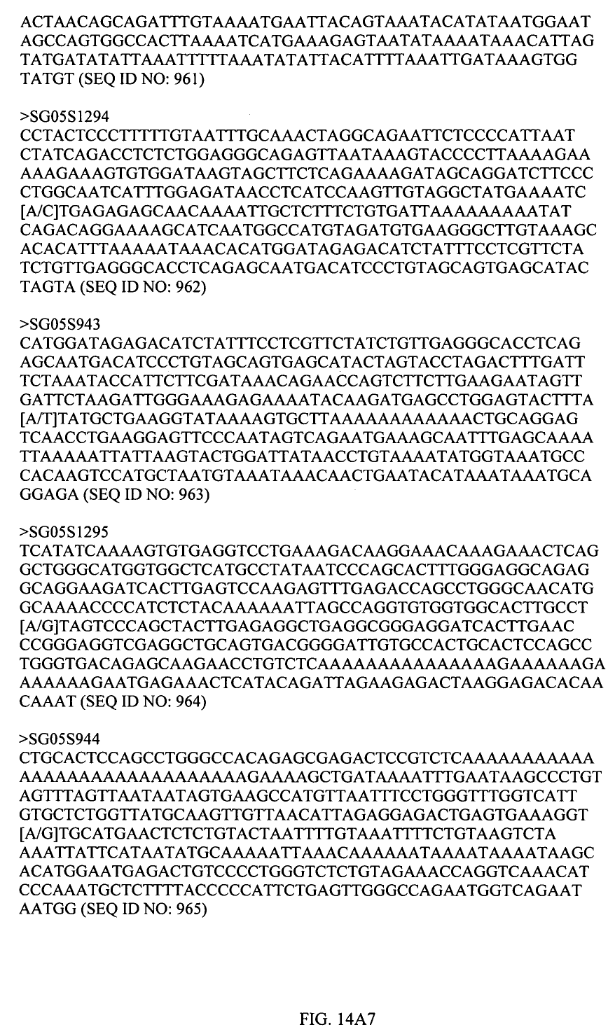

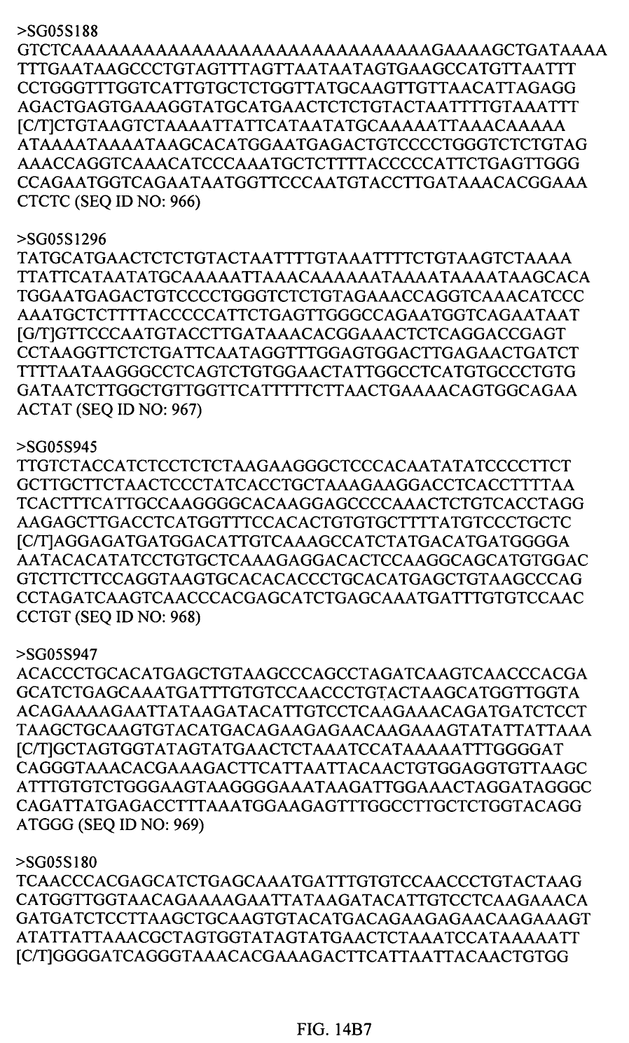

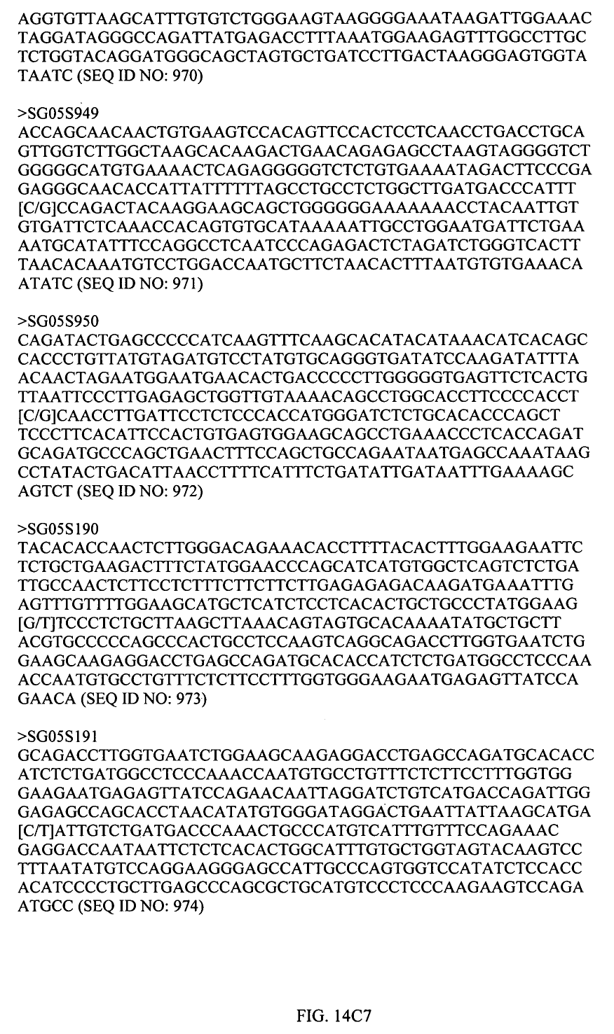

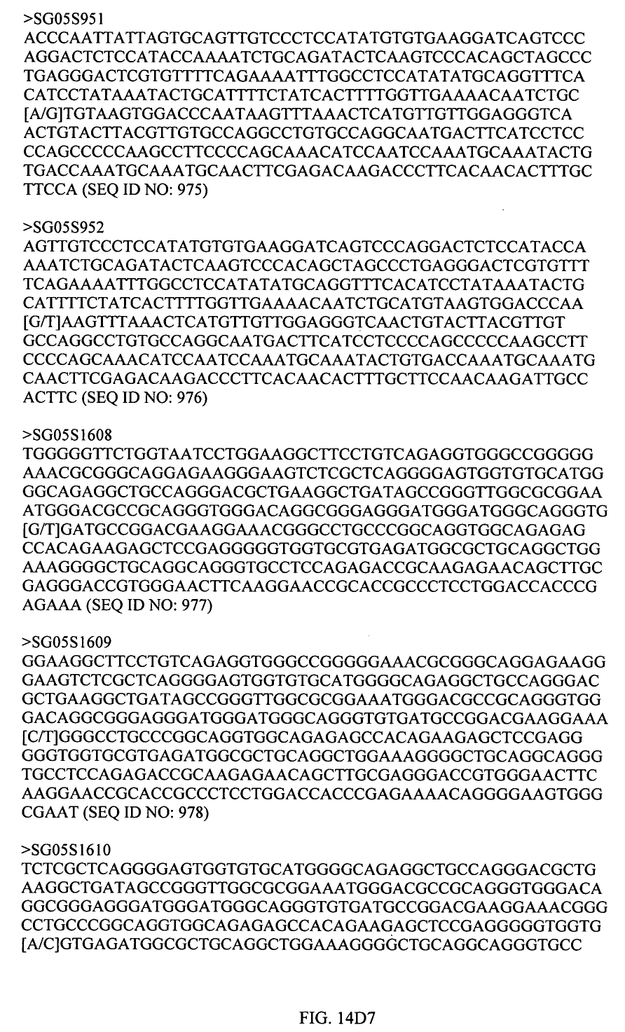

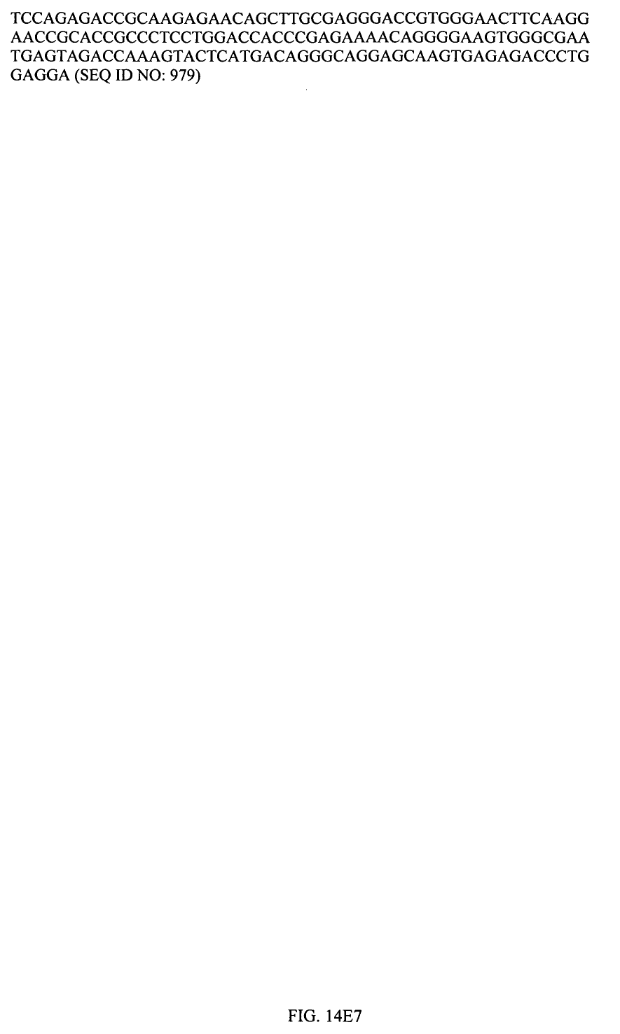

Table 6 is shown in FIGS. 13A-13E4. The table shows the DNA sequence of the microsatellites employed for the CO₅ locus wide association (including Build 33 locations and Y=C or T; S=C or G; R=A or G; W=A or T; M=A or C; K=G or T.)

Table 7 also shown in FIGS. 13A-13X3 shows the DNA sequence of the microsatellites employed for the association studies across KChIP1 (including Build 33 locations).

TABLE 8
The Build 33 location and size of KChIP1 exons.

| EXON | START (NBCI33) | END (B33) | Size (bp) |
|---|---|---|---|
| 1a | 169716298 | 169716511 | 214 |
| UTR 1 | 169848417 | 169848523 | 107 |
| UTR 2 | 169861083 | 169861154 | 72 |
| UTR 3 | 169864589 | 169864679 | 91 |
| UTR 4 | 169867066 | 169867173 | 108 |
| 1b | 169867120 | 169867180 | 61 |
| Ins-r | 170075401 | 170075433 | 33 |
| 2 | 170081305 | 170081429 | 125 |
| 3 | 170082868 | 170082937 | 70 |
| 4 | 170084380 | 170084450 | 71 |
| 5 | 170085260 | 170085367 | 108 |
| 6 | 170095347 | 170095451 | 105 |
| 7 | 170096383 | 170096445 | 63 |
| 8 | 170098306 | 170099177 | 872 |

TABLE 9
The Build 33 location of SNPs found across KChIP1
after the first round of sequencing that was limited
to the exons and flanking sequences.

| START (B33) | MARKER | VARIATION |
|---|---|---|
| 169716197 | KCP_e1a_249924 | C/G |
| 169716300 | KCP_e1a_250027 | C/T |
| 169716322 | KCP_e1a_250049 | A/C |
| 169740666 | KNB_24222 | A/G |
| 169740703 | KNB_24259 | A/G |
| 169741172 | KNB_24728 | G/T |
| 169746339 | KNB_29895 | C/T |
| 169747941 | KNB_31497 | A/G |
| 169751742 | KNB_35298 | A/T |
| 169751814 | KNB_35370 | C/G |
| 169751843 | KNB_35399 | A/G |
| 169848476 | KCP_UTR1_382206 | C/T |
| 169848542 | KCP_UTR1_382272 | A/C |
| 169861338 | KCP_3UTR2_395068 | A/G |
| 169864750 | KCP_3UTR3_398480 | C/T |
| 169864875 | KCP_3UTR3_398605 | C/T |
| 169866182 | KCP_e1b_399912 | G/T |
| 170081292 | KCP_1152 | C/T |
| 170081464 | KCP_1324 | G/C |
| 170081473 | KCP_1333 | A/G |
| 170082789 | KCP_2649 | C/T |
| 170085097 | KCP_4957 | C/T |
| 170085116 | KCP_4976 | C/T |
| 170085151 | KCP_5011 | A/T |
| 170085191 | KCP_5051 | C/T |
| 170085217 | KCP_5077 | A/T |
| 170085342 | KCP_5202 | A/C |
| 170095344 | KCP_15204 | C/T |
| 170095540 | KCP_15400 | C/T |
| 170096292 | KCP_16152 | A/G |
| 170098209 | KCP_18069 | C/T |

Table 10 showing the DNA sequence of the SNPs identified across KChIP1 is also shown in FIGS. 13A-13X3.

TABLE 11
The Build 33 location of SNPs and microsatellites employed
for the first-pass association analysis across KChIP1.

| | | Public | | |
|---|---|---|---|---|
| Start (B33) | Marker | Alias | deCODE alias | Variation |
| 169788696 | DG5S47 | | | |
| 169794522 | DG5S1592 | | | |
| 169843903 | DG5S119 | | | |
| 169869845 | rs933656 | rs933656 | DG00AAFCS | A/G |
| 169869955 | rs2339091 | rs2339091 | DG00AAFCI | G/T |
| 169961410 | DG5S13 | | | |
| 169964087 | rs905808 | rs905808 | SG05S1212 | C/T |
| 170006645 | rs883849 | rs883849 | SG05S206 | A/G |
| 170015858 | DG5S123 | | | |
| 170037283 | rs2135046 | rs2135046 | SG05S159 | C/T |
| 170041996 | DG5S124 | | | |
| 170056955 | rs2339139 | rs2339139 | DG00AAFCR | A/G |
| 170064881 | rs329468 | rs329468 | SG05S896 | A/G |
| 170070041 | rs50057 | rs50057 | SG05S1270 | A/G |
| 170070735 | rs102685 | rs102685 | SG05S905 | C/T |
| 170073252 | rs50364 | rs50364 | DG00AAFCD | A/G |
| 170081292 | KCP_1152 | | SG05S176 | C/T |
| 170081473 | KCP_1333 | | SG05S921 | A/G |
| 170082789 | KCP_2649 | | SG05S923 | C/T |
| 170085116 | KCP_4976 | | SG05S187 | C/T |
| 170085217 | KCP_5077 | | SG05S179 | A/T |
| 170095540 | KCP_15400 | | SG05S946 | C/T |
| 170096292 | KCP_16152 | rs4868018 | SG05S948 | A/G |
| 170098209 | KCP_18069 | rs1363712 | SG05S189 | C/T |
| 170105556 | D5S625 | | | |

TABLE 12
The Build 33 location of SNPs found through sequencing
across KChIP1 (from exon 1b to exon 8).

| | Project | DECODE | SEQ PROJECT | PUBLIC | |
|---|---|---|---|---|---|
| Build 33 Pos | Pos | ALIAS | ALIAS | ALIAS | SNP |
| 169866787 | 9677 | SG05S2107 | KCP_9677 | rs6555900 | C/G |
| 169867465 | 10355 | SG05S229 | KCP_10355 | | A/T |
| 169867556 | 10446 | DG00AAHAR | KCP_10446 | | C/G |
| 169871957 | 14847 | SG05S485 | KCP_14847 | rs4867608 | A/T |
| 169872129 | 15019 | SG05S1298 | KCP_15019 | rs4867973 | A/G |
| 169872417 | 15307 | SG05S437 | KCP_15307 | | A/C |
| 169872421 | 15311 | SG05S438 | KCP_15311 | | A/T |
| 169872435 | 15325 | SG05S439 | KCP_15325 | | C/G |
| 169872949 | 15839 | SG05S440 | KCP_15839 | | A/G |
| 169873539 | 16429 | SG05S486 | KCP_16429 | | C/T |
| 169873680 | 16570 | SG05S487 | KCP_16570 | | A/G |
| 169875123 | 18013 | SG05S488 | KCP_18013 | | A/T |
| 169875568 | 18458 | SG05S1002 | KCP_18458 | rs6555901 | A/G |
| 169876302 | 19192 | SG05S489 | KCP_19192 | | A/G |
| 169878365 | 21255 | SG05S490 | KCP_21255 | | G/T |
| 169878734 | 21624 | SG05S491 | KCP_21624 | rs4867609 | A/G |
| 169879678 | 22568 | SG05S492 | KCP_22568 | | A/C |
| 169879717 | 22607 | SG05S493 | KCP_22607 | | C/T |
| 169881496 | 24386 | SG05S494 | KCP_24386 | | A/G |
| 169882681 | 25571 | SG05S495 | KCP_25571 | | A/C |
| 169883265 | 26155 | SG05S496 | KCP_26155 | rs7443451 | A/G |
| 169883333 | 26223 | SG05S497 | KCP_26223 | | C/G |
| 169883413 | 26303 | SG05S498 | KCP_26303 | | A/G |
| 169883465 | 26355 | SG05S1171 | KCP_26355 | | C/G |
| 169883518 | 26408 | SG05S499 | KCP_26408 | | A/T |
| 169883738 | 26628 | SG05S500 | KCP_26628 | | A/G |
| 169883811 | 26701 | SG05S501 | KCP_26701 | | A/G |
| 169884084 | 26974 | SG05S1172 | KCP_26974 | | C/T |
| 169884145 | 27035 | SG05S502 | KCP_27035 | | G/T |
| 169884439 | 27329 | SG05S503 | KCP_27329 | | C/T |
| 169884682 | 27572 | SG05S504 | KCP_27572 | | A/G |
| 169884707 | 27597 | DG00AAJHT | KCP_27597 | | A/G |
| 169884973 | 27863 | SG05S505 | KCP_27863 | | A/G |
| 169885005 | 27895 | SG05S506 | KCP_27895 | | A/G |
| 169888453 | 31343 | SG05S507 | KCP_31343 | rs4867975 | C/T |
| 169889433 | 32323 | SG05S60 | KCP_32323 | | C/T |
| 169889680 | 32570 | SG05S508 | KCP_32570 | | A/G |
| 169890025 | 32915 | SG05S509 | KCP_32915 | | A/G |
| 169890055 | 32945 | SG05S1173 | KCP_32945 | rs6873409 | A/G |
| 169890089 | 32979 | SG05S1174 | KCP_32979 | rs6873133 | A/C |
| 169890291 | 33181 | SG05S510 | KCP_33181 | rs6873872 | A/G |
| 169892122 | 35012 | SG05S1175 | KCP_35012 | | A/C |
| 169892332 | 35222 | SG05S511 | KCP_35222 | rs7724503 | A/G |
| 169892524 | 35414 | SG05S61 | KCP_35414 | | G/T |
| 169892619 | 35509 | SG05S512 | KCP_35509 | rs6885463 | C/T |
| 169892687 | 35577 | SG05S513 | KCP_35577 | | G/T |
| 169893157 | 36047 | SG05S514 | KCP_36047 | rs6555903 | C/T |
| 169893169 | 36059 | SG05S515 | KCP_36059 | rs6555904 | C/T |
| 169893871 | 36761 | SG05S516 | KCP_36761 | | A/C |
| 169894061 | 36951 | SG05S517 | KCP_36951 | | A/G |
| 169894358 | 37248 | SG05S518 | KCP_37248 | | C/G |
| 169895507 | 38397 | SG05S1176 | KCP_38397 | | C/T |
| 169895699 | 38589 | SG05S953 | KCP_38589 | | A/C |
| 169896322 | 39212 | SG05S519 | KCP_39212 | rs7737732 | G/T |
| 169896357 | 39247 | SG05S520 | KCP_39247 | | A/G |
| 169896369 | 39259 | SG05S521 | KCP_39259 | | A/G |
| 169896451 | 39341 | SG05S1177 | KCP_39341 | | A/G |
| 169896647 | 39537 | SG05S522 | KCP_39537 | | C/T |
| 169896750 | 39640 | SG05S523 | KCP_39640 | | A/T |
| 169896914 | 39804 | SG05S524 | KCP_39804 | | A/G |
| 169897484 | 40374 | SG05S525 | KCP_40374 | | C/T |
| 169897594 | 40484 | SG05S526 | KCP_40484 | | A/G |
| 169897621 | 40511 | SG05S527 | KCP_40511 | | C/T |
| 169897856 | 40746 | SG05S528 | KCP_40746 | | C/T |
| 169898205 | 41095 | SG05S529 | KCP_41095 | | C/T |
| 169898252 | 41142 | SG05S530 | KCP_41142 | | C/T |
| 169898371 | 41261 | SG05S531 | KCP_41261 | | A/G |
| 169899446 | 42336 | SG05S532 | KCP_42336 | | A/G |
| 169899693 | 42583 | SG05S533 | KCP_42583 | | A/G |
| 169900156 | 43046 | SG05S534 | KCP_43046 | | A/G |
| 169900425 | 43315 | SG05S1178 | KCP_43315 | | C/G |
| 169900629 | 43519 | SG05S535 | KCP_43519 | | C/T |
| 169902212 | 45102 | SG05S536 | KCP_45102 | rs2112601 | A/G |
| 169902400 | 45290 | SG05S537 | KCP_45290 | | G/T |
| 169903206 | 46096 | SG05S538 | KCP_46096 | | C/T |
| 169903615 | 46505 | SG05S539 | KCP_46505 | | C/T |
| 169903676 | 46566 | SG05S540 | KCP_46566 | | A/C |
| 169903766 | 46656 | SG05S541 | KCP_46656 | | A/C |
| 169904530 | 47420 | SG05S542 | KCP_47420 | | C/T |
| 169904757 | 47647 | SG05S543 | KCP_47647 | | A/G |
| 169906262 | 49152 | SG05S1179 | KCP_49152 | | A/G |
| 169906576 | 49466 | SG05S544 | KCP_49466 | | A/G |
| 169906846 | 49736 | SG05S545 | KCP_49736 | | A/T |
| 169907866 | 50756 | SG05S1180 | KCP_50756 | | A/G |
| 169908937 | 51827 | SG05S1181 | KCP_51827 | | C/T |
| 169909190 | 52080 | SG05S1182 | KCP_52080 | | C/T |
| 169910099 | 52989 | SG05S546 | KCP_52989 | | A/G |
| 169910133 | 53023 | SG05S547 | KCP_53023 | | C/T |
| 169911784 | 54674 | SG05S548 | KCP_54674 | | A/C |
| 169911823 | 54713 | SG05S549 | KCP_54713 | | A/C |
| 169913086 | 55976 | SG05S1183 | KCP_55976 | | A/G |
| 169913415 | 56305 | SG05S62 | KCP_56305 | | A/G |
| 169913670 | 56560 | SG05S954 | KCP_56560 | | C/T |
| 169913988 | 56878 | SG05S550 | KCP_56878 | | C/G |
| 169914731 | 57621 | SG05S551 | KCP_57621 | | A/G |
| 169914887 | 57777 | SG05S552 | KCP_57777 | | A/G |
| 169915597 | 58487 | SG05S553 | KCP_58487 | | A/G |
| 169917130 | 60020 | SG05S554 | KCP_60020 | | C/T |
| 169917579 | 60469 | SG05S555 | KCP_60469 | | A/G |
| 169917813 | 60703 | SG05S556 | KCP_60703 | | A/G |
| 169919206 | 62096 | SG05S557 | KCP_62096 | | A/G |
| 169919909 | 62799 | SG05S233 | KCP_62799 | | C/T |
| 169921008 | 63898 | SG05S558 | KCP_63898 | | A/G |
| 169921407 | 64297 | SG05S559 | KCP_64297 | | A/G |
| 169921917 | 64807 | SG05S560 | KCP_64807 | | G/T |
| 169922010 | 64900 | SG05S1184 | KCP_64900 | | A/G |
| 169922309 | 65199 | SG05S955 | KCP_65199 | | A/G |
| 169922397 | 65287 | SG05S561 | KCP_65287 | | G/T |
| 169923449 | 66339 | SG05S562 | KCP_66339 | | A/G |
| 169923611 | 66501 | SG05S563 | KCP_66501 | | A/G |
| 169924005 | 66895 | SG05S564 | KCP_66895 | | A/G |
| 169925422 | 68312 | SG05S956 | KCP_68312 | | A/C |
| 169926039 | 68929 | SG05S565 | KCP_68929 | | C/T |
| 169926454 | 69344 | SG05S566 | KCP_69344 | | A/G |
| 169926756 | 69646 | SG05S567 | KCP_69646 | | C/T |
| 169927013 | 69903 | SG05S568 | KCP_69903 | | A/G |
| 169927893 | 70783 | SG05S569 | KCP_70783 | | C/T |
| 169928063 | 70953 | SG05S570 | KCP_70953 | | A/T |
| 169928076 | 70966 | SG05S571 | KCP_70966 | | A/C |
| 169928444 | 71334 | SG05S572 | KCP_71334 | | C/T |
| 169928522 | 71412 | SG05S573 | KCP_71412 | | A/T |
| 169928555 | 71445 | SG05S1185 | KCP_71445 | | C/T |
| 169928665 | 71555 | SG05S1186 | KCP_71555 | | C/T |
| 169928700 | 71590 | SG05S1187 | KCP_71590 | | C/T |
| 169929635 | 72525 | SG05S574 | KCP_72525 | rs4269297 | A/G |
| 169929849 | 72739 | SG05S575 | KCP_72739 | | C/G |
| 169930171 | 73061 | SG05S576 | KCP_73061 | rs4867613 | C/T |
| 169930506 | 73396 | SG05S577 | KCP_73396 | | A/T |
| 169930538 | 73428 | SG05S578 | KCP_73428 | rs4867978 | A/G |
| 169930644 | 73534 | SG05S579 | KCP_73534 | rs4867979 | C/T |
| 169931073 | 73963 | SG05S580 | KCP_73963 | | C/G |
| 169931425 | 74315 | SG05S581 | KCP_74315 | | A/G |
| 169931663 | 74553 | SG05S582 | KCP_74553 | | G/T |
| 169931670 | 74560 | SG05S583 | KCP_74560 | | C/T |
| 169932137 | 75027 | SG05S584 | KCP_75027 | | C/T |
| 169932696 | 75586 | SG05S585 | KCP_75586 | rs7723669 | A/C |
| 169932998 | 75888 | SG05S586 | KCP_75888 | | C/T |
| 169933181 | 76071 | SG05S587 | KCP_76071 | rs386758 | A/G |
| 169933212 | 76102 | SG05S588 | KCP_76102 | rs386759 | C/T |
| 169933256 | 76146 | SG05S589 | KCP_76146 | | A/G |
| 169933389 | 76279 | SG05S1188 | KCP_76279 | rs4368746 | C/T |
| 169933420 | 76310 | SG05S590 | KCP_76310 | | C/T |
| 169933699 | 76589 | SG05S591 | KCP_76589 | | C/T |
| 169933756 | 76646 | SG05S592 | KCP_76646 | | C/T |
| 169934348 | 77238 | SG05S593 | KCP_77238 | | G/T |
| 169934429 | 77319 | SG05S594 | KCP_77319 | | C/G |
| 169934556 | 77446 | SG05S595 | KCP_77446 | | C/T |
| 169934663 | 77553 | SG05S596 | KCP_77553 | | C/T |
| 169934751 | 77641 | SG05S597 | KCP_77641 | rs4242157 | A/G |
| 169934936 | 77826 | SG05S598 | KCP_77826 | | C/G |
| 169934949 | 77839 | SG05S599 | KCP_77839 | rs7735198 | A/G |
| 169935134 | 78024 | SG05S600 | KCP_78024 | rs4867981 | A/G |
| 169935240 | 78130 | SG05S601 | KCP_78130 | rs4867614 | C/T |
| 169935254 | 78144 | SG05S602 | KCP_78144 | | A/C |
| 169935713 | 78603 | SG05S603 | KCP_78603 | | C/T |
| 169935892 | 78782 | SG05S604 | KCP_78782 | | A/G |
| 169935939 | 78829 | SG05S605 | KCP_78829 | | A/G |
| 169935989 | 78879 | SG05S606 | KCP_78879 | | C/T |
| 169936272 | 79162 | SG05S607 | KCP_79162 | | C/T |
| 169936275 | 79165 | SG05S608 | KCP_79165 | | C/T |
| 169936329 | 79219 | SG05S609 | KCP_79219 | | G/T |
| 169936495 | 79385 | SG05S610 | KCP_79385 | rs6876518 | C/T |
| 169936910 | 79800 | SG05S611 | KCP_79800 | | C/G |
| 169937029 | 79919 | SG05S1189 | KCP_79919 | | A/G |
| 169937270 | 80160 | SG05S612 | KCP_80160 | | A/G |
| 169937896 | 80786 | SG05S613 | KCP_80786 | | A/G |
| 169938126 | 81016 | SG05S614 | KCP_81016 | | C/T |
| 169938400 | 81290 | SG05S615 | KCP_81290 | | A/G |
| 169938894 | 81784 | SG05S1190 | KCP_81784 | | A/G |
| 169939578 | 82468 | SG05S957 | KCP_82468 | rs4242158 | A/G |
| 169940311 | 83201 | SG05S616 | KCP_83201 | | C/T |
| 169940995 | 83885 | SG05S617 | KCP_83885 | | A/G |
| 169941106 | 83996 | SG05S618 | KCP_83996 | rs4867615 | A/G |
| 169941897 | 84787 | SG05S1191 | KCP_84787 | | A/T |
| 169942667 | 85557 | SG05S619 | KCP_85557 | | A/G |
| 169942775 | 85665 | SG05S620 | KCP_85665 | rs6892193 | C/T |
| 169942903 | 85793 | SG05S958 | KCP_85793 | rs6892514 | C/T |
| 169943046 | 85936 | SG05S621 | KCP_85936 | | A/G |
| 169943817 | 86707 | SG05S622 | KCP_86707 | | A/T |
| 169944237 | 87127 | SG05S623 | KCP_87127 | rs6881347 | C/G |
| 169945487 | 88377 | SG05S624 | KCP_88377 | | C/T |
| 169945857 | 88747 | SG05S625 | KCP_88747 | | A/T |
| 169945886 | 88776 | SG05S626 | KCP_88776 | | C/T |
| 169945923 | 88813 | SG05S627 | KCP_88813 | | A/G |
| 169946380 | 89270 | SG05S628 | KCP_89270 | | A/G |
| 169946491 | 89381 | SG05S629 | KCP_89381 | rs4867983 | A/G |
| 169947228 | 90118 | SG05S630 | KCP_90118 | | A/G |
| 169947236 | 90126 | SG05S631 | KCP_90126 | | G/T |
| 169947285 | 90175 | SG05S632 | KCP_90175 | | C/T |
| 169947471 | 90361 | SG05S633 | KCP_90361 | | C/G |
| 169947529 | 90419 | SG05S634 | KCP_90419 | | C/T |
| 169947661 | 90551 | SG05S635 | KCP_90551 | | A/G |
| 169947834 | 90724 | SG05S636 | KCP_90724 | | A/G |
| 169948187 | 91077 | SG05S637 | KCP_91077 | rs6874152 | A/G |
| 169948683 | 91573 | SG05S1192 | KCP_91573 | | A/G |
| 169948703 | 91593 | SG05S1193 | KCP_91593 | | G/T |
| 169948722 | 91612 | SG05S1194 | KCP_91612 | | A/G |
| 169948755 | 91645 | SG05S1195 | KCP_91645 | | C/T |
| 169948788 | 91678 | SG05S1196 | KCP_91678 | | A/G |
| 169948798 | 91688 | SG05S1197 | KCP_91688 | | C/T |
| 169948977 | 91867 | SG05S638 | KCP_91867 | | C/T |
| 169949063 | 91953 | SG05S639 | KCP_91953 | | C/T |
| 169949229 | 92119 | SG05S640 | KCP_92119 | | C/T |
| 169949277 | 92167 | SG05S641 | KCP_92167 | | A/T |
| 169949352 | 92242 | SG05S642 | KCP_92242 | | A/G |
| 169949354 | 92244 | SG05S643 | KCP_92244 | rs4867984 | A/G |
| 169949449 | 92339 | SG05S644 | KCP_92339 | | C/T |
| 169950146 | 93036 | SG05S63 | KCP_93036 | | A/G |
| 169950148 | 93038 | SG05S645 | KCP_93038 | | A/G |
| 169950333 | 93223 | SG05S646 | KCP_93223 | rs4867985 | C/T |
| 169950655 | 93545 | SG05S64 | KCP_93545 | | G/T |
| 169950703 | 93593 | SG05S1198 | KCP_93593 | | C/G |
| 169950754 | 93644 | SG05S654 | KCP_93644 | | G/T |
| 169950844 | 93734 | SG05S655 | KCP_93734 | | C/T |
| 169950855 | 93745 | SG05S656 | KCP_93745 | | G/T |
| 169950892 | 93782 | SG05S1199 | KCP_93782 | | C/G |
| 169950990 | 93880 | SG05S657 | KCP_93880 | | C/T |
| 169951245 | 94135 | SG05S1200 | KCP_94135 | | A/C |
| 169951290 | 94180 | SG05S1201 | KCP_94180 | | A/G |
| 169951422 | 94312 | SG05S658 | KCP_94312 | | A/T |
| 169951577 | 94467 | SG05S659 | KCP_94467 | | A/G |
| 169951689 | 94579 | SG05S660 | KCP_94579 | | A/G |
| 169951702 | 94592 | SG05S661 | KCP_94592 | | A/G |
| 169951831 | 94721 | SG05S662 | KCP_94721 | | C/G |
| 169951838 | 94728 | SG05S663 | KCP_94728 | | A/G |
| 169951848 | 94738 | SG05S664 | KCP_94738 | | C/T |
| 169951855 | 94745 | SG05S665 | KCP_94745 | | A/G |
| 169952144 | 95034 | SG05S1202 | KCP_95034 | | A/G |
| 169952209 | 95099 | SG05S666 | KCP_95099 | | A/C |
| 169952705 | 95595 | SG05S667 | KCP_95595 | | A/G |
| 169952838 | 95728 | SG05S670 | KCP_95728 | | A/G |
| 169952962 | 95852 | SG05S671 | KCP_95852 | | A/G |
| 169953175 | 96065 | SG05S672 | KCP_96065 | | C/G |
| 169953185 | 96075 | SG05S673 | KCP_96075 | rs4354060 | A/G |
| 169953207 | 96097 | SG05S674 | KCP_96097 | rs4374772 | C/G |
| 169953297 | 96187 | SG05S675 | KCP_96187 | | A/G |
| 169953327 | 96217 | SG05S676 | KCP_96217 | | A/G |
| 169953334 | 96224 | SG05S677 | KCP_96224 | | A/G |
| 169953426 | 96316 | SG05S678 | KCP_96316 | rs6862741 | A/G |
| 169953728 | 96618 | SG05S1203 | KCP_96618 | | C/G |
| 169953902 | 96792 | SG05S679 | KCP_96792 | rs4867987 | C/T |
| 169954134 | 97024 | SG05S680 | KCP_97024 | rs4867988 | C/T |
| 169954165 | 97055 | SG05S1204 | KCP_97055 | rs4867989 | C/T |
| 169954260 | 97150 | SG05S1205 | KCP_97150 | | A/G |
| 169954800 | 97690 | SG05S681 | KCP_97690 | rs6868698 | A/T |
| 169954954 | 97844 | DG00AAJIA | KCP_97844 | rs2202438 | A/T |
| 169955450 | 98340 | SG05S682 | KCP_98340 | | C/T |
| 169956638 | 99528 | SG05S683 | KCP_99528 | | A/C |
| 169956932 | 99822 | SG05S684 | KCP_99822 | | C/T |
| 169957089 | 99979 | SG05S685 | KCP_99979 | | A/G |
| 169957538 | 100428 | SG05S1206 | KCP_100428 | | G/T |
| 169958211 | 101101 | SG05S1207 | KCP_101101 | rs4495201 | A/G |
| 169958651 | 101541 | SG05S1208 | KCP_101541 | | A/G |
| 169958784 | 101674 | SG05S686 | KCP_101674 | | A/C |
| 169959085 | 101975 | SG05S687 | KCP_101975 | | A/G |
| 169959172 | 102062 | SG05S1209 | KCP_102062 | | A/T |
| 169959537 | 102427 | SG05S688 | KCP_102427 | | A/G |
| 169959561 | 102451 | SG05S1210 | KCP_102451 | | C/T |
| 169959860 | 102750 | SG05S1211 | KCP_102750 | | C/T |
| 169959992 | 102882 | DG00AAJIB | KCP_102882 | | C/T |
| 169961135 | 104025 | SG05S689 | KCP_104025 | rs4867990 | A/G |
| 169961268 | 104158 | SG05S690 | KCP_104158 | | G/T |
| 169961404 | 104294 | SG05S691 | KCP_104294 | rs4867991 | A/G |
| 169961971 | 104861 | SG05S692 | KCP_104861 | | A/G |
| 169962144 | 105034 | SG05S693 | KCP_105034 | | A/G |
| 169962410 | 105300 | SG05S694 | KCP_105300 | rs4242159 | A/T |
| 169962429 | 105319 | SG05S695 | KCP_105319 | rs4428429 | C/G |
| 169962889 | 105779 | SG05S696 | KCP_105779 | | A/G |
| 169962929 | 105819 | SG05S697 | KCP_105819 | | C/T |
| 169963467 | 106357 | SG05S698 | KCP_106357 | rs4867990 | A/G |
| 169963592 | 106482 | SG05S699 | KCP_106482 | | C/T |
| 169963741 | 106631 | SG05S700 | KCP_106631 | | A/G |
| 169963761 | 106651 | SG05S701 | KCP_106651 | | A/G |
| 169963827 | 106717 | SG05S702 | KCP_106717 | | A/T |
| 169964021 | 106911 | SG05S703 | KCP_106911 | rs905807 | C/G |
| 169964087 | 106977 | SG05S1212 | KCP_106977 | rs905808 | C/T |
| 169964112 | 107002 | SG05S1213 | KCP_107002 | rs905809 | C/T |
| 169964368 | 107258 | SG05S988 | KCP_107258 | rs905811 | A/G |
| 169964490 | 107380 | DG00AAJIC | KCP_107380 | | A/G |
| 169964862 | 107752 | SG05S705 | KCP_107752 | rs905812 | A/T |
| 169964998 | 107888 | SG05S706 | KCP_107888 | | A/T |
| 169965204 | 108094 | SG05S707 | KCP_108094 | | C/T |
| 169965210 | 108100 | SG05S708 | KCP_108100 | | C/T |
| 169965293 | 108183 | SG05S709 | KCP_108183 | | C/T |
| 169965384 | 108274 | SG05S710 | KCP_108274 | | C/T |
| 169965778 | 108668 | SG05S1214 | KCP_108668 | | C/T |
| 169965813 | 108703 | SG05S230 | KCP_108703 | | G/T |
| 169965814 | 108704 | SG05S711 | KCP_108704 | | A/G |
| 169965989 | 108879 | SG05S712 | KCP_108879 | | A/T |
| 169966345 | 109235 | SG05S713 | KCP_109235 | | C/G |
| 169966790 | 109680 | SG05S714 | KCP_109680 | | A/C |
| 169966813 | 109703 | SG05S715 | KCP_109703 | rs6877169 | A/G |
| 169966833 | 109723 | SG05S716 | KCP_109723 | | A/G |
| 169966856 | 109746 | SG05S718 | KCP_109746 | rs905813 | A/G |
| 169967196 | 110086 | SG05S719 | KCP_110086 | | C/T |
| 169967509 | 110399 | SG05S720 | KCP_110399 | | C/G |
| 169968134 | 111024 | SG05S721 | KCP_111024 | | A/C |
| 169968258 | 111148 | SG05S722 | KCP_111148 | rs7726675 | C/T |
| 169968588 | 111478 | SG05S723 | KCP_111478 | rs2089191 | C/G |
| 169968602 | 111492 | SG05S724 | KCP_111492 | | A/G |
| 169968614 | 111504 | SG05S725 | KCP_111504 | | C/G |
| 169969010 | 111900 | SG05S726 | KCP_111900 | | A/G |
| 169969185 | 112075 | SG05S727 | KCP_112075 | | A/G |
| 169969769 | 112659 | SG05S728 | KCP_112659 | rs4867994 | C/T |
| 169970341 | 113231 | SG05S729 | KCP_113231 | | A/G |
| 169970367 | 113257 | SG05S730 | KCP_113257 | rs4867616 | A/G |
| 169970440 | 113330 | SG05S733 | KCP_113330 | | A/G |
| 169971048 | 113938 | SG05S734 | KCP_113938 | | A/G |
| 169971464 | 114354 | SG05S736 | KCP_114354 | | A/G |
| 169971531 | 114421 | SG05S1215 | KCP_114421 | | C/T |
| 169971568 | 114458 | SG05S737 | KCP_114458 | rs2879337 | C/T |
| 169971621 | 114511 | SG05S738 | KCP_114511 | | C/T |
| 169972209 | 115099 | SG05S740 | KCP_115099 | rs1553537 | A/G |
| 169972598 | 115488 | SG05S741 | KCP_115488 | rs6870612 | C/G |
| 169973254 | 116144 | SG05S742 | KCP_116144 | rs1013922 | C/T |
| 169973325 | 116215 | SG05S743 | KCP_116215 | | A/G |
| 169973369 | 116259 | SG05S744 | KCP_116259 | | A/G |
| 169973465 | 116355 | SG05S745 | KCP_116355 | rs2089192 | A/G |
| 169974479 | 117369 | SG05S746 | KCP_117369 | rs870109 | A/T |
| 169974926 | 117816 | SG05S747 | KCP_117816 | rs1553538 | C/T |
| 169976065 | 118955 | SG05S1216 | KCP_118955 | | C/T |
| 169977940 | 120830 | SG05S748 | KCP_120830 | rs905819 | C/T |
| 169978197 | 121087 | SG05S749 | KCP_121087 | | C/T |
| 169978247 | 121137 | SG05S192 | KCP_121137 | | A/G |
| 169978339 | 121229 | SG05S193 | KCP_121229 | | C/T |
| 169978427 | 121317 | SG05S1217 | KCP_121317 | | C/T |
| 169980304 | 123194 | SG05S751 | KCP_123194 | | A/G |
| 169980403 | 123293 | SG05S752 | KCP_123293 | | A/G |
| 169980481 | 123371 | SG05S1218 | KCP_123371 | | A/G |
| 169980664 | 123554 | SG05S753 | KCP_123554 | | C/T |
| 169981035 | 123925 | SG05S1219 | KCP_123925 | | A/G |
| 169981067 | 123957 | SG05S754 | KCP_123957 | | A/G |
| 169981628 | 124518 | SG05S755 | KCP_124518 | | C/T |
| 169981632 | 124522 | SG05S756 | KCP_124522 | | G/T |
| 169981987 | 124877 | SG05S194 | KCP_124877 | rs4146511 | C/T |
| 169982473 | 125363 | SG05S757 | KCP_125363 | rs2202436 | A/T |
| 169982868 | 125758 | SG05S758 | KCP_125758 | | C/T |
| 169983196 | 126086 | SG05S195 | KCP_126086 | rs2202437 | A/G |
| 169983318 | 126208 | DG00AAJHA | KCP_126208 | | T/C |
| 169983565 | 126455 | SG05S1220 | KCP_126455 | | C/G |
| 169983591 | 126481 | SG05S759 | KCP_126481 | rs2221441 | C/G |
| 169983692 | 126582 | SG05S760 | KCP_126582 | | A/G |
| 169985824 | 128714 | SG05S1221 | KCP_128714 | | A/G |
| 169985916 | 128806 | SG05S151 | KCP_128806 | | A/G |
| 169985985 | 128875 | SG05S761 | KCP_128875 | | C/T |
| 169986162 | 129052 | SG05S763 | KCP_129052 | rs4867617 | C/G |
| 169986174 | 129064 | SG05S762 | KCP_129064 | | C/G |
| 169986189 | 129079 | SG05S764 | KCP_129079 | rs4867618 | C/T |
| 169986203 | 129093 | SG05S152 | KCP_129093 | rs4867995 | C/G |
| 169986237 | 129127 | SG05S480 | KCP_129127 | rs4867619 | A/G |
| 169986334 | 129224 | SG05S765 | KCP_129224 | rs486762 | G/T |
| 169986478 | 129368 | SG05S766 | KCP_129368 | | C/G |
| 169986579 | 129469 | SG05S181 | KCP_129469 | | A/G |
| 169986800 | 129690 | SG05S182 | KCP_129690 | rs4867996 | G/T |
| 169986957 | 129847 | SG05S767 | KCP_129847 | rs4867997 | A/G |
| 169986984 | 129874 | SG05S985 | KCP_129874 | rs4867998 | A/C |
| 169986999 | 129889 | SG05S986 | KCP_129889 | rs4867999 | A/G |
| 169987419 | 130309 | DG00AAJHB | KCP_130309 | | A/G |
| 169987667 | 130557 | SG05S196 | KCP_130557 | rs905822 | C/G |
| 169988155 | 131045 | SG05S768 | KCP_131045 | | A/G |
| 169988354 | 131244 | SG05S197 | KCP_131244 | rs905824 | A/G |
| 169988368 | 131258 | SG05S769 | KCP_131258 | rs905825 | C/T |
| 169988581 | 131471 | SG05S770 | KCP_131471 | rs905826 | A/G |
| 169988714 | 131604 | SG05S1222 | KCP_131604 | | A/G |
| 169988812 | 131702 | SG05S771 | KCP_131702 | rs905827 | C/T |
| 169988905 | 131795 | SG05S65 | KCP_131795 | rs4868001 | C/T |
| 169988964 | 131854 | SG05S153 | KCP_131854 | rs6861734 | G/T |
| 169989037 | 131927 | SG05S772 | KCP_131927 | rs6865908 | A/G |
| 169989257 | 132147 | SG05S773 | KCP_132147 | | C/T |
| 169989533 | 132423 | SG05S774 | KCP_132423 | | A/G |
| 169989704 | 132594 | SG05S775 | KCP_132594 | rs4868002 | G/T |
| 169989739 | 132629 | SG05S776 | KCP_132629 | | A/G |
| 169989787 | 132677 | SG05S154 | KCP_132677 | | A/G |
| 169990284 | 133174 | SG05S777 | KCP_133174 | | C/T |
| 169990366 | 133256 | SG05S1223 | KCP_133256 | | A/G |
| 169990548 | 133438 | SG05S778 | KCP_133438 | rs4867621 | A/G |
| 169990840 | 133730 | SG05S779 | KCP_133730 | | C/T |
| 169990962 | 133852 | SG05S780 | kcp_133852 | | A/G |
| 169991155 | 134045 | SG05S198 | KCP_134045 | | C/T |
| 169991415 | 134305 | SG05S199 | KCP_134305 | rs7737768 | C/T |
| 169991521 | 134411 | SG05S781 | KCP_134411 | rs6555907 | C/T |
| 169991729 | 134619 | SG05S1224 | KCP_134619 | | A/C |
| 169991939 | 134829 | SG05S782 | KCP_134829 | | C/T |
| 169992076 | 134966 | SG05S783 | KCP_134966 | | A/G |
| 169992155 | 135045 | SG05S784 | KCP_135045 | | A/G |
| 169992628 | 135518 | SG05S200 | KCP_135518 | rs4868003 | G/T |
| 169992821 | 135711 | SG05S785 | KCP_135711 | | G/T |
| 169993032 | 135922 | SG05S786 | KCP_135922 | | A/G |
| 169993096 | 135986 | SG05S183 | KCP_135986 | | A/G |
| 169993146 | 136036 | SG05S481 | KCP_136036 | | A/C |
| 169993585 | 136475 | SG05S787 | KCP_136475 | | C/T |
| 169994082 | 136972 | SG05S201 | KCP_136972 | rs4868004 | A/G |
| 169994770 | 137660 | SG05S202 | KCP_137660 | | A/G |
| 169995924 | 138814 | SG05S788 | KCP_138814 | | C/T |
| 169997343 | 140233 | SG05S789 | KCP_140233 | | C/T |
| 169997640 | 140530 | SG05S1225 | KCP_140530 | | A/G |
| 169998201 | 141091 | SG05S1226 | KCP_141091 | | A/G |
| 170000256 | 143146 | SG05S1227 | KCP_143146 | rs953601 | C/T |
| 170000611 | 143501 | SG05S1228 | KCP_143501 | | C/T |
| 170000722 | 143612 | SG05S66 | KCP_143612 | rs4867622 | A/G |
| 170000869 | 143759 | SG05S790 | KCP_143759 | | C/T |
| 170000983 | 143873 | SG05S1229 | KCP_143873 | | C/T |
| 170001571 | 144461 | SG05S1230 | KCP_144461 | | C/T |
| 170001578 | 144468 | SG05S1299 | KCP_144468 | rs931805 | C/T |
| 170002070 | 144960 | SG05S203 | KCP_144960 | rs2279873 | C/T |
| 170002435 | 145325 | SG05S791 | KCP_145325 | rs6891256 | C/T |
| 170002801 | 145691 | SG05S1231 | KCP_145691 | | A/G |
| 170003438 | 146328 | SG05S792 | KCP_146328 | | A/G |
| 170003572 | 146462 | SG05S793 | KCP_146462 | | G/T |
| 170003856 | 146746 | SG05S482 | KCP_146746 | | C/T |
| 170003940 | 146830 | SG05S1232 | KCP_146830 | | C/T |
| 170004075 | 146965 | SG05S794 | KCP_146965 | | C/T |
| 170004199 | 147089 | SG05S1233 | KCP_147089 | | C/G |
| 170004733 | 147623 | SG05S204 | KCP_147623 | rs2292146 | C/T |
| 170005151 | 148041 | SG05S795 | KCP_148041 | | C/T |
| 170006326 | 149216 | SG05S205 | KCP_149216 | rs6555908 | A/G |
| 170006485 | 149375 | SG05S796 | KCP_149375 | rs883848 | G/T |
| 170006645 | 149535 | SG05S206 | KCP_149535 | rs883849 | A/G |
| 170006910 | 149800 | SG05S1234 | KCP_149800 | | A/G |
| 170007023 | 149913 | SG05S797 | KCP_149913 | rs4867623 | C/T |
| 170007516 | 150406 | SG05S798 | KCP_150406 | rs4868005 | G/T |
| 170007640 | 150530 | SG05S987 | KCP_150530 | | C/T |
| 170007808 | 150698 | SG05S799 | KCP_150698 | | G/T |
| 170007921 | 150811 | SG05S155 | KCP_150811 | | A/G |
| 170008215 | 151105 | SG05S800 | KCP_151105 | | G/T |
| 170008937 | 151827 | SG05S801 | KCP_151827 | rs2339094 | A/G |
| 170009218 | 152108 | SG05S1235 | KCP_152108 | | A/G |
| 170009587 | 152477 | SG05S802 | KCP_152477 | | C/T |
| 170009592 | 152482 | SG05S803 | KCP_152482 | | A/C |
| 170010385 | 153275 | SG05S1236 | KCP_153275 | rs6866371 | C/T |
| 170010518 | 153408 | SG05S1237 | KCP_153408 | | C/T |
| 170010943 | 153833 | SG05S804 | KCP_153833 | | C/T |
| 170011041 | 153931 | DG00AAJHC | KCP_153931 | rs2879338 | A/G |
| 170011269 | 154159 | SG05S805 | KCP_154159 | | A/G |
| 170011475 | 154365 | SG05S1238 | KCP_154365 | | A/G |
| 170011963 | 154853 | SG05S806 | KCP_154853 | | C/T |
| 170012367 | 155257 | SG05S807 | KCP_155257 | | C/G |
| 170013726 | 156616 | SG05S808 | KCP_156616 | | C/T |
| 170013842 | 156732 | SG05S207 | KCP_156732 | rs924876 | A/T |
| 170015154 | 158044 | SG05S809 | KCP_158044 | | A/G |
| 170015582 | 158472 | SG05S810 | KCP_158472 | | C/T |
| 170015603 | 158493 | SG05S811 | KCP_158493 | | A/G |
| 170015680 | 158570 | SG05S812 | KCP_158570 | | C/T |
| 170015727 | 158617 | SG05S67 | KCP_158617 | rs2036559 | C/T |
| 170016200 | 159090 | SG05S813 | KCP_159090 | rs6889236 | A/G |
| 170016255 | 159145 | SG05S814 | KCP_159145 | | A/G |
| 170016259 | 159149 | SG05S815 | KCP_159149 | | C/T |
| 170016791 | 159681 | SG05S1239 | KCP_159681 | | A/G |
| 170016798 | 159688 | SG05S1240 | KCP_159688 | | A/G |
| 170017255 | 160145 | SG05S208 | KCP_160145 | | A/G |
| 170017524 | 160414 | SG05S816 | KCP_160414 | | G/T |
| 170018297 | 161187 | SG05S817 | KCP_161187 | | A/G |
| 170018356 | 161246 | SG05S818 | KCP_161246 | | C/G |
| 170018549 | 161439 | SG05S819 | KCP_161439 | | A/G |
| 170018573 | 161463 | SG05S820 | KCP_161463 | | C/T |
| 170019258 | 162148 | SG05S821 | KCP_162148 | | C/T |
| 170019314 | 162204 | SG05S1241 | KCP_162204 | | A/C |
| 170019379 | 162269 | SG05S822 | KCP_162269 | | A/T |
| 170019414 | 162304 | SG05S823 | KCP_162304 | | C/G |
| 170019958 | 162848 | SG05S824 | KCP_162848 | | C/G |
| 170020197 | 163087 | SG05S825 | KCP_163087 | rs6871693 | C/G |
| 170020606 | 163496 | SG05S826 | KCP_163496 | | A/G |
| 170020870 | 163760 | SG05S827 | KCP_163760 | | A/G |
| 170021444 | 164334 | SG05S1242 | KCP_164334 | | A/G |
| 170022007 | 164897 | SG05S209 | KCP_164897 | | A/G |
| 170022125 | 165015 | SG05S828 | KCP_165015 | | G/T |
| 170022343 | 165233 | SG05S1243 | KCP_165233 | | C/T |
| 170022545 | 165435 | SG05S1244 | KCP_165435 | | C/T |
| 170023275 | 166165 | SG05S829 | KCP_166165 | | A/G |
| 170024034 | 166924 | SG05S1245 | KCP_166924 | rs4867624 | C/T |
| 170024668 | 167558 | SG05S830 | KCP_167558 | | A/G |
| 170025753 | 168643 | SG05S1246 | KCP_168643 | | A/G |
| 170025970 | 168860 | SG05S1247 | KCP_168860 | rs2202439 | C/G |
| 170026021 | 168911 | SG05S1248 | KCP_168911 | | A/G |
| 170026162 | 169052 | SG05S1249 | KCP_169052 | | A/G |
| 170026344 | 169234 | SG05S156 | KCP_169234 | | A/G |
| 170028032 | 170922 | SG05S1297 | KCP_170922 | rs4868008 | A/C |
| 170028055 | 170945 | SG05S831 | KCP_170945 | | C/G |
| 170028163 | 171053 | SG05S1250 | KCP_171053 | rs4868009 | A/G |
| 170028303 | 171193 | SG05S1300 | KCP_171193 | rs4868010 | G/T |
| 170028752 | 171642 | SG05S1251 | KCP_171642 | | G/T |
| 170028987 | 171877 | SG05S832 | KCP_171877 | | A/G |
| 170030482 | 173372 | SG05S833 | KCP_173372 | | A/G |
| 170030815 | 173705 | SG05S834 | KCP_173705 | | C/T |
| 170030958 | 173848 | SG05S210 | KCP_173848 | | A/G |
| 170030986 | 173876 | SG05S1252 | KCP_173876 | | C/T |
| 170031092 | 173982 | SG05S157 | KCP_173982 | rs6875696 | A/C |
| 170031149 | 174039 | SG05S835 | KCP_174039 | | C/T |
| 170031150 | 174040 | SG05S836 | KCP_174040 | | A/G |
| 170031353 | 174243 | DG00AAJHF | KCP_174243 | | A/G |
| 170031709 | 174599 | SG05S837 | KCP_174599 | | C/T |
| 170031812 | 174702 | SG05S838 | KCP_174702 | | C/T |
| 170031962 | 174852 | SG05S839 | KCP_174852 | | A/G |
| 170031972 | 174862 | SG05S840 | KCP_174862 | rs4628005 | G/T |
| 170032216 | 175106 | SG05S158 | KCP_175106 | rs2339095 | C/G |
| 170032280 | 175170 | SG05S211 | KCP_175170 | rs6555910 | A/G |
| 170032361 | 175251 | SG05S841 | KCP_175251 | | C/T |
| 170032362 | 175252 | DG00AAJHG | KCP_175252 | rs7721722 | A/G |
| 170032610 | 175500 | SG05S842 | KCP_175500 | | A/G |
| 170032814 | 175704 | SG05S843 | KCP_175704 | | A/G |
| 170033021 | 175911 | SG05S844 | KCP_175911 | | A/G |
| 170033923 | 176813 | SG05S845 | KCP_176813 | | A/G |
| 170033946 | 176836 | DG00AAJHH | KCP_176836 | | A/G |
| 170034620 | 177510 | SG05S184 | KCP_177510 | rs4868011 | A/C |
| 170034720 | 177610 | SG05S1253 | KCP_177610 | | G/T |
| 170034980 | 177870 | SG05S846 | KCP_177870 | | G/T |
| 170035009 | 177899 | SG05S847 | KCP_177899 | rs4868012 | C/T |
| 170036929 | 179819 | SG05S848 | KCP_179819 | | C/T |
| 170037010 | 179900 | SG05S1254 | KCP_179900 | | G/T |
| 170037283 | 180173 | SG05S159 | KCP_180173 | rs2135046 | C/T |
| 170037347 | 180237 | SG05S212 | KCP_180237 | rs2135047 | C/G |
| 170038967 | 181857 | SG05S1255 | KCP_181857 | | C/T |
| 170039237 | 182127 | SG05S1256 | KCP_182127 | | C/T |
| 170039419 | 182309 | SG05S849 | KCP_182309 | | A/T |
| 170041190 | 184080 | SG05S160 | KCP_184080 | rs2292147 | C/G |
| 170041385 | 184275 | SG05S964 | KCP_184275 | | A/G |
| 170042689 | 185579 | DG00AAJDX | KCP_185579 | | C/A |
| 170043158 | 186048 | SG05S213 | KCP_186048 | | A/G |
| 170043789 | 186679 | SG05S161 | KCP_186679 | | C/G |
| 170043953 | 186843 | SG05S850 | KCP_186843 | | A/C |
| 170043997 | 186887 | SG05S965 | KCP_186887 | | C/T |
| 170044226 | 187116 | DG00AAJDY | KCP_187116 | | A/G |
| 170044277 | 187167 | SG05S851 | KCP_187167 | | C/G |
| 170044368 | 187258 | SG05S162 | KCP_187258 | | G/T |
| 170044661 | 187551 | SG05S853 | KCP_187551 | | A/G |
| 170044798 | 187688 | DG00AAJDZ | KCP_187688 | | T/A |
| 170044904 | 187794 | SG05S966 | KCP_187794 | | C/T |
| 170045075 | 187965 | SG05S967 | KCP_187965 | | C/T |
| 170046043 | 188933 | SG05S968 | KCP_188933 | | C/T |
| 170046441 | 189331 | SG05S214 | KCP_189331 | | A/G |
| 170047120 | 190010 | SG05S854 | KCP_190010 | rs2221442 | A/G |
| 170047129 | 190019 | SG05S855 | KCP_190019 | | C/G |
| 170048070 | 190960 | SG05S856 | KCP_190960 | | C/G |
| 170048074 | 190964 | SG05S857 | KCP_190964 | | C/T |
| 170048090 | 190980 | SG05S858 | KCP_190980 | | C/G |
| 170048315 | 191205 | SG05S859 | KCP_191205 | rs4868015 | C/T |
| 170048733 | 191623 | SG05S860 | KCP_191623 | | A/G |
| 170049238 | 192128 | SG05S990 | KCP_192128 | | C/T |
| 170049852 | 192742 | DG00AAJEB | KCP_192742 | rs1973529 | T/C |
| 170050303 | 193193 | DG00AAJEC | KCP_193193 | | G/A |
| 170051066 | 193956 | SG05S163 | KCP_193956 | rs2202440 | C/T |
| 170051438 | 194328 | SG05S861 | KCP_194328 | | A/T |
| 170051462 | 194352 | SG05S862 | KCP_194352 | | A/G |
| 170051726 | 194616 | DG00AAJEE | KCP_194616 | rs2036560 | T/C |
| 170051899 | 194789 | SG05S970 | KCP_194789 | | C/T |
| 170052012 | 194902 | SG05S863 | KCP_194902 | | A/G |
| 170052171 | 195061 | SG05S971 | KCP_195061 | | G/T |
| 170052988 | 195878 | SG05S864 | KCP_195878 | | C/T |
| 170053658 | 196548 | DG00AAJEF | KCP_196548 | | A/G |
| 170053669 | 196559 | SG05S865 | KCP_196559 | rs7702368 | A/G |
| 170053840 | 196730 | SG05S866 | KCP_196730 | | G/T |
| 170053939 | 196829 | SG05S867 | KCP_196829 | | C/G |
| 170054581 | 197471 | SG05S972 | KCP_197471 | | A/G |
| 170054620 | 197510 | SG05S973 | KCP_197510 | | C/T |
| 170054788 | 197678 | DG00AAJEG | KCP_197678 | rs962804 | T/C |
| 170054803 | 197693 | SG05S884 | KCP_197693 | | A/G |
| 170054885 | 197775 | DG00AAJEH | KCP_197775 | | C/T |
| 170055781 | 198671 | DG00AAJEI | KCP_198671 | | A/G |
| 170055957 | 198847 | SG05S974 | KCP_198847 | | A/G |
| 170056043 | 198933 | DG00AAJEJ | KCP_198933 | | G/A |
| 170056137 | 199027 | SG05S975 | KCP_199027 | | A/G |
| 170056475 | 199365 | DG00AAJEK | KCP_199365 | rs6555911 | A/G |
| 170056516 | 199406 | SG05S164 | KCP_199406 | rs6887777 | A/T |
| 170056578 | 199468 | SG05S1257 | KCP_199468 | | C/T |
| 170057283 | 200173 | SG05S165 | KCP_200173 | | C/G |
| 170057351 | 200241 | DG00AAJEL | KCP_200241 | | A/G |
| 170057605 | 200495 | SG05S976 | KCP_200495 | | A/G |
| 170057933 | 200823 | SG05S991 | KCP_200823 | | A/C |
| 170058193 | 201083 | SG05S992 | KCP_201083 | rs4464713 | C/T |
| 170058699 | 201589 | SG05S885 | KCP_201589 | | C/T |
| 170059095 | 201985 | DG00AAJEM | KCP_201985 | | G/A |
| 170059177 | 202067 | DG00AAJEN | KCP_202067 | rs2221440 | A/G |
| 170059203 | 202093 | SG05S977 | KCP_202093 | | A/C |
| 170059905 | 202795 | DG00AAJEO | KCP_202795 | rs875184 | C/T |
| 170060219 | 203109 | SG05S1258 | KCP_203109 | | A/G |
| 170060292 | 203182 | SG05S978 | KCP_203182 | | A/G |
| 170060393 | 203283 | SG05S979 | KCP_203283 | rs905818 | A/G |
| 170061018 | 203908 | SG05S980 | KCP_203908 | rs905817 | C/T |
| 170061292 | 204182 | SG05S981 | KCP_204182 | rs872435 | G/T |
| 170061352 | 204242 | SG05S166 | KCP_204242 | rs6897344 | C/T |
| 170061419 | 204309 | SG05S982 | KCP_204309 | | A/G |
| 170061618 | 204508 | SG05S983 | KCP_204508 | rs872436 | A/G |
| 170061670 | 204560 | SG05S1259 | KCP_204560 | | A/G |
| 170061727 | 204617 | SG05S984 | KCP_204617 | rs6876574 | C/T |
| 170061799 | 204689 | SG05S1260 | KCP_204689 | rs905816 | G/T |
| 170061809 | 204699 | SG05S1261 | KCP_204699 | | A/T |
| 170061845 | 204735 | SG05S1262 | KCP_204735 | rs905815 | C/T |
| 170062696 | 205586 | SG05S886 | KCP_205586 | rs329466 | C/T |
| 170062747 | 205637 | SG05S887 | KCP_205637 | rs7721804 | A/C |
| 170062756 | 205646 | SG05S888 | KCP_205646 | rs329467 | C/T |
| 170062777 | 205667 | SG05S889 | KCP_205667 | rs7721817 | A/G |
| 170062940 | 205830 | SG05S167 | KCP_205830 | | C/T |
| 170062950 | 205840 | SG05S890 | KCP_205840 | | A/G |
| 170063305 | 206195 | SG05S891 | KCP_206195 | | C/G |
| 170063313 | 206203 | SG05S892 | KCP_206203 | | C/T |
| 170063377 | 206267 | SG05S168 | KCP_206267 | | A/G |
| 170063732 | 206622 | SG05S893 | KCP_206622 | rs7727631 | A/G |
| 170063817 | 206707 | SG05S894 | KCP_206707 | | C/T |
| 170063983 | 206873 | SG05S1263 | KCP_206873 | rs7710016 | A/T |
| 170064013 | 206903 | SG05S1264 | KCP_206903 | | C/G |
| 170064648 | 207538 | SG05S895 | KCP_207538 | | C/T |
| 170064760 | 207650 | SG05S969 | KCP_207650 | | A/G |
| 170064771 | 207661 | SG05S169 | KCP_207661 | | C/G |
| 170064881 | 207771 | SG05S896 | KCP_207771 | rs329468 | A/G |
| 170065075 | 207965 | SG05S170 | KCP_207965 | | C/T |
| 170065694 | 208584 | SG05S171 | KCP_208584 | | A/G |
| 170065711 | 208601 | SG05S232 | KCP_208601 | rs329469 | A/C |
| 170065715 | 208605 | SG05S897 | KCP_208605 | | A/G |
| 170065740 | 208630 | SG05S172 | KCP_208630 | | C/T |
| 170065834 | 208724 | SG05S1265 | KCP_208724 | rs7700434 | C/T |
| 170066123 | 209013 | SG05S1266 | KCP_209013 | rs7734240 | C/T |
| 170066260 | 209150 | SG05S1267 | KCP_209150 | | A/G |
| 170067967 | 210857 | SG05S898 | KCP_210857 | rs2194162 | A/G |
| 170068018 | 210908 | SG05S899 | KCP_210908 | | C/G |
| 170068420 | 211310 | SG05S900 | KCP_211310 | | A/G |
| 170068510 | 211400 | SG05S901 | KCP_211400 | rs410348 | A/G |
| 170068614 | 211504 | SG05S902 | KCP_211504 | | A/G |
| 170068635 | 211525 | SG05S173 | KCP_211525 | | A/G |
| 170068731 | 211621 | SG05S903 | KCP_211621 | | A/G |
| 170068759 | 211649 | SG05S1268 | KCP_211649 | | G/T |
| 170068960 | 211850 | SG05S185 | KCP_211850 | rs329470 | C/T |
| 170069885 | 212775 | SG05S186 | KCP_212775 | rs4349730 | A/G |
| 170070003 | 212893 | SG05S1269 | KCP_212893 | rs6877532 | G/T |
| 170070041 | 212931 | SG05S1270 | KCP_212931 | rs50057 | A/G |
| 170070593 | 213483 | SG05S904 | KCP_213483 | | A/G |
| 170070700 | 213590 | SG05S1271 | KCP_213590 | rs102684 | C/T |
| 170070735 | 213625 | SG05S905 | KCP_213625 | rs102685 | C/T |
| 170070768 | 213658 | SG05S1272 | KCP_213658 | rs102686 | A/G |
| 170071584 | 214474 | SG05S1273 | KCP_214474 | rs329471 | C/G |
| 170071665 | 214555 | SG05S1274 | KCP_214555 | rs329472 | C/T |
| 170071715 | 214605 | SG05S1275 | KCP_214605 | rs329473 | C/G |
| 170072023 | 214913 | SG05S1276 | KCP_214913 | | A/G |
| 170072363 | 215253 | SG05S906 | KCP_215253 | rs4041562 | C/T |
| 170072373 | 215263 | SG05S907 | KCP_215263 | rs172944 | C/T |
| 170072484 | 215374 | SG05S908 | KCP_215374 | | A/G |
| 170072485 | 215375 | SG05S909 | KCP_215375 | | A/G |
| 170072562 | 215452 | SG05S910 | KCP_215452 | rs191297 | A/G |
| 170072712 | 215602 | SG05S1277 | KCP_215602 | rs186646 | A/C |
| 170072813 | 215703 | SG05S174 | KCP_215703 | | A/C |
| 170073179 | 216069 | SG05S1278 | KCP_216069 | | C/T |
| 170073555 | 216445 | SG05S1279 | KCP_216445 | rs1363709 | A/G |
| 170073565 | 216455 | SG05S1280 | KCP_216455 | rs329474 | C/G |
| 170074202 | 217092 | SG05S993 | KCP_217092 | rs984559 | A/G |
| 170074303 | 217193 | SG05S994 | KCP_217193 | | C/T |
| 170074359 | 217249 | SG05S995 | KCP_217249 | rs329475 | A/G |
| 170075932 | 218822 | SG05S996 | KCP_218822 | | A/G |
| 170076291 | 219181 | SG05S997 | KCP_219181 | | A/G |
| 170076439 | 219329 | SG05S998 | KCP_219329 | rs801987 | C/G |
| 170077257 | 220147 | SG05S911 | KCP_220147 | | A/T |
| 170078779 | 221669 | SG05S912 | KCP_221669 | | C/G |
| 170078881 | 221771 | SG05S1281 | KCP_221771 | | C/T |
| 170078909 | 221799 | DG00AAJHJ | KCP_221799 | rs7733559 | A/T |
| 170078966 | 221856 | SG05S913 | KCP_221856 | rs7713498 | C/T |
| 170079102 | 221992 | SG05S1282 | KCP_221992 | | C/T |
| 170079170 | 222060 | SG05S175 | KCP_222060 | | C/T |
| 170079176 | 222066 | SG05S1283 | KCP_222066 | | A/T |
| 170079986 | 222876 | SG05S1284 | KCP_222876 | | A/G |
| 170080026 | 222916 | SG05S914 | KCP_222916 | | C/T |
| 170080378 | 223268 | SG05S915 | KCP_223268 | rs4868017 | C/T |
| 170080480 | 223370 | SG05S916 | KCP_223370 | | C/T |
| 170080678 | 223568 | SG05S917 | KCP_223568 | | G/T |
| 170080917 | 223807 | SG05S918 | KCP_223807 | | C/G |
| 170081127 | 224017 | SG05S919 | KCP_224017 | rs6555913 | A/G |
| 170081263 | 224153 | SG05S1285 | KCP_224153 | | G/T |
| 170081464 | 224354 | SG05S920 | KCP_224354 | | C/G |
| 170081779 | 224669 | SG05S231 | KCP_224669 | | A/C |
| 170082330 | 225220 | SG05S177 | KCP_225220 | | A/G |
| 170082361 | 225251 | SG05S1286 | KCP_225251 | | A/T |
| 170082496 | 225386 | SG05S922 | KCP_225386 | | C/T |
| 170083131 | 226021 | SG05S1287 | KCP_226021 | | A/C |
| 170083226 | 226116 | SG05S1288 | KCP_226116 | | C/G |
| 170083558 | 226448 | SG05S924 | KCP_226448 | | A/G |
| 170083941 | 226831 | SG05S925 | KCP_226831 | | A/G |
| 170084576 | 227466 | SG05S926 | KCP_227466 | | C/T |
| 170084823 | 227713 | SG05S927 | KCP_227713 | | A/G |
| 170084981 | 227871 | SG05S178 | KCP_227871 | | C/G |
| 170085097 | 227987 | SG05S483 | KCP_227987 | rs2277951 | C/T |
| 170085116 | 228006 | SG05S187 | KCP_228006 | rs2277952 | C/T |
| 170085151 | 228041 | SG05S928 | KCP_228041 | | A/T |
| 170085191 | 228081 | SG05S929 | KCP_228081 | | C/T |
| 170085217 | 228107 | SG05S179 | KCP_228107 | | A/T |
| 170085834 | 228724 | SG05S1289 | KCP_228724 | | A/G |
| 170086059 | 228949 | SG05S999 | KCP_228949 | | C/T |
| 170086143 | 229033 | SG05S1000 | KCP_229033 | | C/T |
| 170086250 | 229140 | SG05S1001 | KCP_229140 | | C/T |
| 170086709 | 229599 | SG05S930 | KCP_229599 | | A/C |
| 170086826 | 229716 | SG05S931 | KCP_229716 | | C/T |
| 170087721 | 230611 | SG05S932 | KCP_230611 | rs6894038 | C/G |
| 170087734 | 230624 | SG05S933 | KCP_230624 | rs6894316 | A/G |
| 170087780 | 230670 | SG05S934 | KCP_230670 | rs6875006 | G/T |
| 170087950 | 230840 | SG05S1290 | KCP_230840 | | A/G |
| 170088932 | 231822 | SG05S1291 | KCP_231822 | rs1422978 | C/T |
| 170089182 | 232072 | SG05S1292 | KCP_232072 | rs2194160 | C/T |
| 170089631 | 232521 | SG05S1293 | KCP_232521 | rs1592987 | A/T |
| 170090569 | 233459 | SG05S935 | KCP_233459 | rs6870201 | A/G |
| 170090765 | 233655 | SG05S989 | KCP_233655 | rs2032863 | A/G |
| 170091557 | 234447 | SG05S936 | KCP_234447 | rs6876375 | A/G |
| 170091681 | 234571 | SG05S937 | KCP_234571 | | C/T |
| 170091700 | 234590 | SG05S938 | KCP_234590 | | A/T |
| 170092075 | 234965 | SG05S939 | KCP_234965 | | C/T |
| 170092275 | 235165 | SG05S940 | KCP_235165 | rs1363710 | G/T |
| 170092318 | 235208 | SG05S941 | KCP_235208 | rs1363711 | A/G |
| 170092468 | 235358 | SG05S942 | KCP_235358 | | A/G |
| 170093047 | 235937 | SG05S1294 | KCP_235937 | | A/C |
| 170093362 | 236252 | SG05S943 | KCP_236252 | | A/T |
| 170094119 | 237009 | SG05S1295 | KCP_237009 | | A/G |
| 170094581 | 237471 | SG05S944 | KCP_237471 | rs1422979 | A/G |
| 170094615 | 237505 | SG05S188 | KCP_237505 | rs4867628 | C/T |
| 170094780 | 237670 | SG05S1296 | KCP_237670 | | G/T |
| 170095344 | 238234 | SG05S945 | KCP_238234 | | C/T |
| 170095662 | 238552 | SG05S947 | KCP_238552 | | C/T |
| 170095701 | 238591 | SG05S180 | KCP_238591 | | C/T |
| 170096774 | 239664 | SG05S949 | KCP_239664 | | C/G |
| 170097477 | 240367 | SG05S950 | KCP_240367 | rs6879997 | C/G |
| 170098637 | 241527 | SG05S190 | KCP_241527 | rs1363713 | G/T |
| 170098914 | 241804 | SG05S191 | KCP_241804 | rs1055381 | C/T |
| 170099451 | 242341 | SG05S951 | KCP_242341 | rs1363714 | A/G |
| 170099467 | 242357 | SG05S952 | KCP_242357 | rs6872337 | G/T |
| 170106814 | | SG05S1608 | SG05S1608 | rs1544762 | G/T |
| 170106833 | | SG05S1609 | SG05S1609 | | C/T |
| 170106887 | | SG05S1610 | SG05S1610 | | A/C |

TABLE 13
The Build 33 location of SNPs and microsatellites employed
for the subsequent association analysis across KChIP1.

| | | | | Vari- |
|---|---|---|---|---|
| Start (B33) | Marker | Public alias | deCODE alias | ation |
| 169477886 | rs1895301 | rs1895301 | SG05S2143 | C/T |
| 169500972 | rs1422752 | rs1422752 | SG05S1616 | C/T |
| 169518355 | rs1422754 | rs1422754 | SG05S1617 | A/G |
| 169653708 | DG5S1173 | | | |
| 169661202 | DG5S44 | | | |
| 169673519 | SG05S872 | rs6881730 | SG05S872 | A/G |
| 169678485 | SG05S873 | rs925080 | SG05S873 | A/G |
| 169693772 | DG5S45 | | | |
| 169696877 | KCP_rs315773 | rs315773 | SG05S76 | A/G |
| 169702377 | DG5S46 | | | |
| 169705506 | SG05S876 | rs315757 | SG05S876 | A/G |
| 169709736 | KCP_rs952767 | rs952767 | SG05S79 | G/T |
| 169740666 | KNB_24222 | rs314155 | SG05S1611 | A/G |
| 169740703 | KNB_24259 | | DG00AAIGF | A/G |
| 169741172 | KNB_24728 | rs2656842 | DG00AAIGG | G/T |
| 169745438 | DG5S1178 | | | |
| 169746339 | KNB_29895 | | DG00AAIGH | C/T |
| 169747941 | KNB_31497 | | DG00AAIGI | A/G |
| 169751742 | KNB_35298 | rs2075612 | DG00AAIGZ | A/T |
| 169751814 | KNB_35370 | | DG00AAIHA | C/G |
| 169751843 | KNB_35399 | rs703508 | DG00AAIHB | A/G |
| 169753660 | KCP_rs314129 | rs314129 | SG05S83 | C/T |
| 169782203 | KCP_rs183398 | rs183398 | SG05S87 | C/T |
| 169788696 | DG5S47 | | | |
| 169794522 | DG5S1592 | | | |
| 169815996 | rs1032856 | rs1032856 | SG05S96 | C/G |
| 169833941 | rs2055606 | rs2055606 | SG05S1621 | C/T |
| 169843903 | DG5S119 | | | |
| 169859275 | KCP_rs888934 | rs888934 | SG05S93 | A/G |
| 169867465 | KCP_10355 | | SG05S229 | A/T |
| 169867556 | KCP_10446 | | DG00AAHAR | C/G |
| 169869845 | rs933656 | rs933656 | DG00AAFCS | A/G |
| 169869955 | rs2339091 | rs2339091 | DG00AAFCI | G/T |
| 169890996 | rs1862331 | rs1862331 | DG00AAFCL | C/T |
| 169895699 | KCP_38589 | | SG05S953 | A/C |
| 169922309 | KCP_65199 | | SG05S955 | A/G |
| 169939578 | KCP_82468 | rs4242158 | SG05S957 | A/G |
| 169942903 | KCP_85793 | rs6892514 | SG05S958 | C/T |
| 169950655 | KCP_93545 | | SG05S64 | G/T |
| 169951970 | DG5S955 | | | |
| 169954954 | KCP_97844 | rs2202438 | DG00AAJIA | A/T |
| 169959992 | KCP_102882 | | DG00AAJIB, | C/T |
| | | | SG05S2479 | |
| 169961410 | DG5S13 | | | |
| 169964490 | KCP_107380 | | DG00AAJIC | A/G |
| 169965813 | KCP_108703 | | SG05S230 | G/T |
| 169981987 | KCP_124877 | rs4146511 | SG05S194 | C/T |
| 169983196 | KCP_126086 | rs2202437 | SG05S195 | A/G |
| 169983318 | KCP_126208 | | DG00AAJHA | T/C |
| 169986203 | KCP_129093 | rs4867995 | SG05S152 | C/G |
| 169986237 | KCP_129127 | rs4867619 | SG05S480 | A/G |
| 169986800 | KCP_129690 | rs4867996 | SG05S182 | G/T |
| 169987419 | KCP_130309 | | DG00AAJHB | A/G |
| 169987667 | KCP_130557 | rs905822 | SG05S196 | C/G |
| 169987873 | rs905823 | rs905823 | SG05S1302 | A/C |
| 169988354 | KCP_131244 | rs905824 | SG05S197 | A/G |
| 169988964 | KCP_131854 | rs6861734 | SG05S153 | G/T |
| 169989787 | KCP_132677 | | SG05S154 | A/G |
| 169991155 | KCP_134045 | | SG05S198 | C/T |
| 169992628 | KCP_135518 | rs4868003 | SG05S200 | G/T |
| 169993146 | KCP_136036 | | SG05S481 | A/C |
| 169994770 | KCP_137660 | | SG05S202 | A/G |
| 170000722 | KCP_143612 | rs4867622 | SG05S66 | A/G |
| 170002070 | KCP_144960 | rs2279873 | SG05S203 | C/T |
| 170003856 | KCP_146746 | | SG05S482 | C/T |
| 170006326 | KCP_149216 | rs6555908 | SG05S205 | A/G |
| 170006645 | KCP_149535 | rs883849 | SG05S206 | A/G |
| 170006645 | rs883849 | rs883849 | SG05S206 | A/G |
| 170013842 | KCP_156732 | rs924876 | SG05S207 | A/T |
| 170015727 | KCP_158617 | rs2036559 | SG05S67 | C/T |
| 170015858 | DG5S123 | | | |
| 170017255 | KCP_160145 | | SG05S208 | A/G |
| 170022007 | KCP_164897 | | SG05S209 | A/G |
| 170026344 | KCP_169234 | | SG05S156 | A/G |
| 170030958 | KCP_173848 | | SG05S210 | A/G |
| 170031092 | KCP_173982 | rs6875696 | SG05S157 | A/C |
| 170031353 | KCP_174243 | | DG00AAJHF | A/G |
| 170032216 | KCP_175106 | rs2339095 | SG05S158 | C/G |
| 170032280 | KCP_175170 | rs6555910 | SG05S211 | A/G |
| 170032362 | KCP_175252 | rs7721722 | DG00AAJHG | A/G |
| 170033946 | KCP_176836 | | DG00AAJHH | A/G |
| 170037283 | KCP_180173 | rs2135046 | SG05S159 | C/T |
| 170037283 | rs2135046 | rs2135046 | SG05S159 | C/T |
| 170037347 | KCP_180237 | rs2135047 | SG05S212 | C/G |
| 170041190 | KCP_184080 | rs2292147 | SG05S160 | C/G |
| 170041996 | DG5S124 | | | |
| 170042689 | KCP_185579 | | DG00AAJDX | C/A |
| 170043158 | KCP_186048 | | SG05S213 | A/G |
| 170043789 | KCP_186679 | | SG05S161 | C/G |
| 170044226 | KCP_187116 | | DG00AAJDY | A/G |
| 170044368 | KCP_187258 | | SG05S162 | G/T |
| 170044798 | KCP_187688 | | DG00AAJDZ | T/A |
| 170046441 | KCP_189331 | | SG05S214 | A/G |
| 170049852 | KCP_192742 | rs1973529 | DG00AAJEB | T/C |
| 170050303 | KCP_193193 | | DG00AAJEC | G/A |
| 170051066 | KCP_193956 | rs2202440 | SG05S163 | C/T |
| 170051726 | KCP_194616 | rs2036560 | DG00AAJEE | T/C |
| 170053658 | KCP_196548 | | DG00AAJEF | A/G |
| 170054788 | KCP_197678 | rs962804 | DG00AAJEG | T/C |
| 170054885 | KCP_197775 | | DG00AAJEH | C/T |
| 170056043 | KCP_198933 | | DG00AAJEJ | G/A |
| 170056475 | KCP_199365 | rs6555911 | DG00AAJEK | A/G |
| 170056955 | rs2339139 | rs2339139 | DG00AAFCR | A/G |
| 170057351 | KCP_200241 | | DG00AAJEL | A/G |
| 170059095 | KCP_201985 | | DG00AAJEM | G/A |
| 170059177 | KCP_202067 | rs2221440 | DG00AAJEN | A/G |
| 170059905 | KCP_202795 | rs875184 | DG00AAJEO | C/T |
| 170061292 | rs872435 | rs872435 | SG05S981 | G/T |
| 170061352 | KCP_204242 | rs6897344 | SG05S166 | C/T |
| 170063377. | KCP_206267 | | SG05S168 | A/G |
| 170064771 | KCP_207661 | | SG05S169 | C/G |
| 170064881 | rs329468 | rs329468 | SG05S896 | A/G |
| 170065075 | KCP_207965 | | SG05S170 | C/T |
| 170068635 | KCP_211525 | | SG05S173 | A/G |
| 170068960 | KCP_211850 | rs329470 | SG05S185 | C/T |
| 170069885 | KCP_212775 | rs4349730 | SG05S186 | A/G |
| 170070041 | rs50057 | rs50057 | SG05S1270 | A/G |
| 170073252 | rs50364 | rs50364 | DG00AAFCD | A/G |
| 170078909 | KCP_221799 | rs7733559 | DG00AAJHJ | A/T |
| 170080678 | KCP_223568 | | SG05S917 | G/T |
| 170081292 | KCP_1152 | | SG05S176 | C/T |
| 170081473 | KCP_1333 | | SG05S921 | A/G |
| 170082330 | KCP_225220 | | SG05S177 | A/G |
| 170082789 | KCP_2649 | | SG05S923 | C/T |
| 170084981 | KCP_227871 | | SG05S178 | C/G |
| 170085097 | KCP_227987 | rs2277951 | SG05S483 | C/T |
| 170085115 | KCP_4976 | | SG05S187 | C/T |
| 170085217 | KCP_228107 | | SG05S179 | A/T |
| 170085217 | KCP_5077 | | SG05S179 | A/T |
| 170089631 | KCP_232521 | rs1592987 | SG05S1293 | A/T |
| 170090765 | KCP_233655 | rs2032863 | SG05S989 | A/G |
| 170094615 | KCP_237505 | rs4867628 | SG05S188 | C/T |
| 170095540 | KCP_15400 | | SG05S946 | C/T |
| 170095701 | KCP_238591 | | SG05S180 | C/T |
| 170096292 | KCP_16152 | rs4868018 | SG05S948 | A/G |
| 170098209 | KCP_241099 | rs1363712 | SG05S189 | C/T |
| 170098209 | KCP_18069 | rs1363712 | SG05S189 | C/T |
| 170098637 | KCP_241527 | rs1363713 | SG05S190 | G/T |
| 170098914 | KCP_241804 | rs1055381 | SG05S191 | C/T |
| 170105556 | D5S625 | | | |
| 170167429 | DG5S959 | | | |
| 170361737 | rs1551583 | rs1551583 | SG05S1619 | C/G |
| 170389497 | rs1457692 | rs1457692 | SG05S1618 | A/G |

In order to define SNP-only haplotypes, 66 SNPs (Bold entries in Tables 12 and 13) were further genotyped totalling 948 diabetic patients (538 with BMI<30; 410 with BMI>=30) and 570 controls across 600 kb of KChIP1, of which 58 were concentrated in the 231 kb region encompassing exon 1b, the large intron (where Hap D1 resides) through to exon 8. The most significant 7-SNP haplotype (Hap S7—see Table 14) observed in non-obese Type II diabetes. (p=1.33×10⁻⁶) is significantly correlated with D1 (D′=0.76 between Hap D1 and Hap S7) and captures approximately 75% of the chromosomes that carry Hap D1. The relative risk of this 280 kb haplotype for all diabetes patients is 1.77, with a carrier frequency of 40%.

Hap S7 can be made more specific by adding more SNPs, e.g., by adding DG00AAJEH, the relative risk increases to 2.28 in all diabetic patients vs controls. This variant of Hap S7, denoted Hap S7′, has a carrier frequency of 20.1% in all diabetes patients and population attributable risk (PAR)=12.3%

TABLE 14
Alleles contained within Hap S7 and Hap S7′

| | Length | | | | | | | | |
|---|---|---|---|---|---|---|---|---|---|
| Haplotype | (kb) | | | | | | | | |
| Hap S7 | 280 | G | rs1032856 | G | KCP_rs888934 | T | KCP_93545 | C | KCP_102882 |
| | | G | KCP_169234 | G | KCP_186048 | | | A | KCP_16152 |
| Hap S7′ | 280 | G | rs1032856 | G | KCP_rs888934 | T | KCP_93545 | C | KCP_102882 |
| | | G | KCP_169234 | G | KCP_186048 | C | KCP_197775 | A | KCP_16152 |

TABLE 15
Association analysis of Hap S7 (Hap E) and Hap S7′ (HAP E′) in type 2 diabetes.

| | p-val | r | #aff | aff. freq | aff. freq (carr) | #con | con. freq | con. freq (carr) | info |
|---|---|---|---|---|---|---|---|---|---|
| Hap E | | | | | | | | | |
| T2D BMI < 30 | 1.33E−06 | 1.929 | 525 | 0.2549 | 0.379841195 | 527 | 0.1506 | 0.255828099 | 0.654 |
| T2D BMI > 30 | 0.015813 | 1.451 | 387 | 0.1959 | 0.315039082 | 527 | 0.1437 | 0.24617045 | 0.670 |
| T2D All | 5.04E−06 | 1.769 | 912 | 0.2270 | 0.350961655 | 527 | 0.1424 | 0.244220163 | 0.661 |
| T2D Males | 1.56E−05 | 1.831 | 526 | 0.2337 | 0.358157968 | 527 | 0.1428 | 0.244772025 | 0.650 |
| T2D Females | 0.001484 | 1.623 | 386 | 0.2187 | 0.34178225 | 527 | 0.1471 | 0.250938707 | 0.651 |
| Hap E′ | | | | | | | | | |
| T2D BMI < 30 | 0.000185 | 2.248 | 518 | 0.1229 | 0.215573079 | 453 | 0.0587 | 0.110434655 | 0.571 |
| T2D BMI > 30 | 0.015423 | 1.896 | 379 | 0.0966 | 0.174484759 | 453 | 0.0534 | 0.101016667 | 0.517 |
| T2D All | 0.000105 | 2.279 | 897 | 0.1130 | 0.20051463 | 453 | 0.0530 | 0.100301178 | 0.535 |
| T2D Males | 0.000551 | 2.243 | 517 | 0.1098 | 0.195525378 | 453 | 0.0521 | 0.098826539 | 0.545 |
| T2D Females | 0.004482 | 1.976 | 380 | 0.1101 | 0.195946622 | 453 | 0.0589 | 0.110893515 | 0.564 |

Additional Analysis of Haplotypes in the KChIP1 Region.
Additional SNP Genotyping for KChIP1 Gene

By increasing the haplotype diversity studied within the LD block spanning the KChIP1 gene through typing additional markers, additional haplotypes made up of only SNPs that correlated with D1 or were independent were expected. A 232 kb region was sequenced encompassing the full length KChIP1 gene in 94 Icelandic patients and controls in order to search for additional SNPs for genotyping. None of the SNPs identified caused a non-synonymous change in the coding sequence pointing to the uncommonly conserved nature of the primary structure of KChIP1. Of the 716 SNPs identified in this effort and validated public SNPs either side of the sequenced region, 66 were selected for further genotyping in 802 unrelated Icelandic T2D patients (447 with BMI<30) and 570 population-based controls. The remaining SNPs were excluded from further typing as they either completely correlated with the selected SNPs and therefore were redundant with respect to the information they would provide, or the SNP had a very low minor allele frequency (<5%). The 66 SNPs are listed in Table 25. The LD plot of the 66 SNPs is in FIG. 12 and agrees with the LD structure derived by the HapMap project in a Caucasian population.

The most significant SNP-only haplotype observed, when searching for haplotypes containing up to 3 SNPs, was haplotype S3 (p-value=1.36×10−6; aff allelic freq=59.8%; RR=1.61; PAR=40.1%) (G SG05S96 C DG00AAJIB C DG00AAJHF) that survived our adjustment for multiple testing for all possible three-marker haplotypes (p-value=0.04). With obese Type II diabetes patients, the RR was 1.47 (p-value=2.89×10⁻⁴; aff allelic freq=57.5%; PAR=38.0%) (FIG. 12). D1 is highly correlated to haplotype S3.

This significant result was followed with an expansion of the haplotype up to and including 7 markers. Haplotype S7 was (G SG05S96 G SG05S93 T SG05S64 C DG00AAJIB G SG05S156 G SG05S213 A SG05S948) observed with high correlation to S3 but with higher relative risk (p-value=2.33×10⁻⁷; aff allelic freq=25.6%; RR=2.05; PAR=24.6%). With obese Type II diabetes patients, the RR was 1.52 (p-value=0.009; aff allelic freq=19.6%; PAR=12.9%).

KChIP1 is Expressed in Pancreatic β-Cells

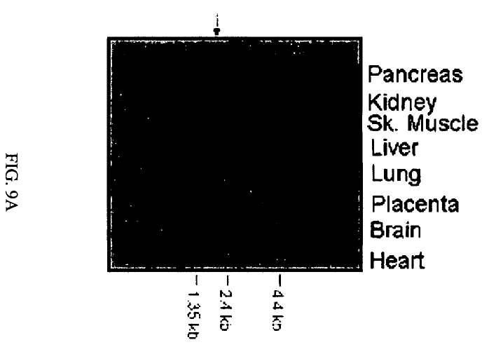

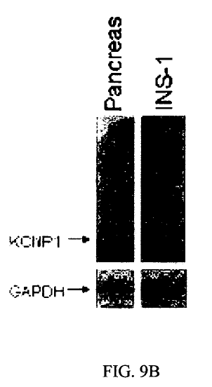

KChIP1 was initially identified in brain through yeast two-hybrid screens using Kv4.3 as a bait. By Northern blot analysis we detected expression in brain and somewhat less expression in pancreas and kidney (FIG. 9A). Western blot analysis detected KChIP in insulin-secreting rat pancreatic β-cell line, INS-1, but not in whole-pancreas homogenates. This is consistent with specific localization to β-cells as KChIP1 was most likely not detected in pancreatic whole lysates since islets constitute only 2-3% of the pancreas (FIG. 9B).

Loss of Function of KChIP1 Increases Insulin Secretion in Pancreatic β-Cells

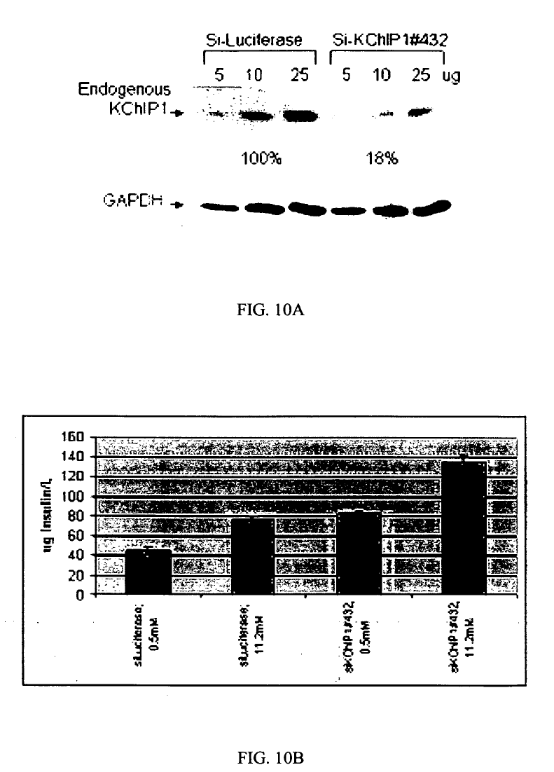

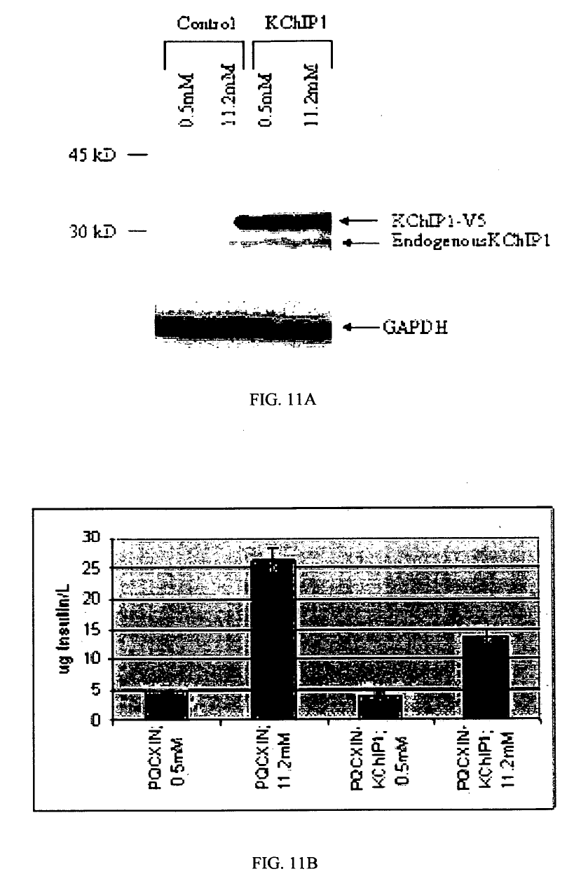

When viewed in the context of previous data indicating its role in regulating the function of Kv4 type voltage-gated potassium channels and the known role of the K_{ATP} channel in propagating insulin secretion, the expression of KChIP1 in pancreatic cells suggested to us that KChIP1 might play a role in control of insulin secretion. To assess the requirement of KChIP1 for insulin secretion KChIP1 was knocked down by RNA interference in rat pancreatic β-cells INS-1 cells (FIG. 9B). We obtained the best knockdown of KChIP1 using a siRNA targeted to the 3′ end of the mRNA encoding the C-terminus of KChIP1 (shKChIP1#sh432). The siRNA was delivered into INS-1 cells using the retroviral vector pSIREN-RetroQ; this vector is designed to stably express a small interfering dsRNA (siRNA) from the human U6 promoter. As a control, we used INS-1 cells expressing an irrelevant luciferase siRNA (shLuc) from a hairpin construct or a retrovirus that expresses the GFP protein. Based on Western blot analysis, the estimated silencing of KChIP1 expression for the INS1-shKChIP1#432 cells was 82% compared to INS1-shLuc cells (100%) (FIG. 10A).

The effect of reduced KChIP1 expression in INS-1 cells on insulin secretion was analyzed by comparing insulin secretion in INS-1 cell lines stably transduced with either pSIREN-RetroQ-shKChIP1 or control vector pSIREN-RetroQ-shLuc. The amount of secreted insulin was determined both at the basal levels and after challenging with 11.2 mM glucose. In INS-1-shKChIP1#432 cells, basal insulin secretion was increased 1.8 fold (two-sided: p-value=1.84×10⁻⁷) and after glucose stimulation was increased 1.8 fold (two-sided: p-value=1.89×10⁻⁶) when compared to INS-1-shLuc cells (FIG. 10B). The unrelated siRNA for luciferase did not affect either basal secretion or glucose induced secretion when compared to INS-1 cells expressing GFP (data not shown).

The putative role of KChIP1 in insulin secretion was also evaluated by overexpressing KChIP1 in INS-1 cells using retroviral mediated gene transfer. INS-1 cells were stably transduced with either the pQCXIN-KChIP1 retrovirus expressing the human KChIP1 cDNA or an empty pQCXIN retrovirus as control. Western blot analysis demonstrated that the production of the KChIP1 protein from the retrovirus was in excess over the endogenous KChIP1 protein (FIG. 11A). The control cells, INS-1-pQCXIN, showed a 5-fold increase in insulin release in response to a glucose challenge. In contrast, glucose stimulated insulin secretion was reduced almost two-fold (50%) (p-value=5.22×10⁻⁵) in KChIP1 overexpressing INS-1 cells when compared to the control (INS-1 pQXCIN) (FIG. 11B). The basal insulin secretion was reduced very slightly in KChIP1 overexpressing cells when compared to the control cells. The expression of KChIP1 was unchanged under the different experimental conditions (basal vs. glucose stimulated) (FIG. 11A).

These results indicate that KChIP1 is a negative regulator of insulin secretion by INS-1 cells, although these experiments do not show whether KChIP1 has a direct or an indirect effect on insulin secretion. The KChIP1 gene has not, to date, been implicated in the pathogenesis of Type II diabetes. Isolation of this Type II diabetes gene was achieved by genome-wide linkage analysis with microsatellite markers and subsequent locus-wide association analysis of the 5q34-q35.2 locus with additional microsatellite markers. By comparing the frequencies of individual alleles and haplotypes between the two cohorts, the location of the putative disease gene was narrowed down to a few hundred kilobases. Subsequently, gene assessment was carried out using SNPs and additional microsatellites typed in all the genes in and around the observed region of association. The gene that showed strongest association with non-obese Type II diabetes was KChIP1, encoding the Kv-channel interacting protein 1 (KChIP1). This demonstrates a strategy of the usefulness of highly informative microsatellite markers to complement SNP markers in association studies that has additionally been used in the isolation of genes for myocardial infarction, ischemic stroke, osteoporosis, and schizophrenia. Each microsatellite exhibits a diversity of alleles with a range of frequencies that facilitate cost-effective studies of haplotype diversity within any given set of LD blocks.

Two independent disease-associated haplotypes within the KChIP1 gene, haplotype S3 and D2 are reported. Haplotype S3 is significant in Iceland even after correction for multiple testing through randomization of the phenotype. D2 was first found to show significant association to Type II diabetes in the Danish cohort even after correction for multiple markers. This haplotype showed a crisp replication in the Icelandic cohort. The two haplotypes appear to be independent and are additive when contributing to Type II diabetes. Over 60% of the Icelandic and Danish Type II diabetes patients carry one or both haplotypes. No underlying mutations within coding exons were found that capture a substantial portion of the risk of the overlying disease haplotype; therefore, these variants are most likely surrogates for underlying functional variants that affect transcription, splicing, message stability, transport or translation efficiency.

Analyzing the expression pattern of KChIP1 in diabetes relevant target tissues revealed that the gene has a limited tissue distribution. In lysates of whole pancreas the protein was not observed because it was most likely below the level of detection. The islets of Langerhans account for only 2-3% of the pancreas; however, KChIP1 was readily detected in pure rat α-cell lysates. Therefore, functional assays were used to test the hypothesis that KChIP1 plays a role in α-cell function suc has insulin secretion. Employing stable cell lines, in which endogenous KChIP1 has been knocked down by approximately 80% using siRNA interference, it was demonstrated that the loss of KChIP1 expression results in an increase in both basal and glucose-stimulated insulin secretion. Conversely, over-expression of KChIP1 reduces insulin secretion stimulated by glucose. This showed that KChIP1 has an inhibitory effect on insulin secretion which has not been reported previously. Thus, KChIP1 either directly or indirectly exerts a negative influence on insulin secretion.

The hypothesis that KChIP1 plays a role in Type II diabetes is supported by showing that haplotype S3 is significantly correlated with serum insulin levels in non-medicated GAD negative Type II diabetes patients. It was important to remove GAD positive patients since they are more likely to have a form of diabetes closer to Type 1 DM with inflammatory β-cell destruction (LADA). The study was applied to a subcohort of Type II diabetes patients who had insulin measurements while not on oral diabetes medications or insulin as they will affect insulin levels. Taken together with the β-cells functional this data suggests that the disease associated haplotypes may directly lead to increased KChIP1 expression in pancreatic β-cells thus causing impaired insulin secretion in response to glucose. The susceptibility gene that we have isolated influences insulin Type II diabetes secretion supporting the hypothesis of a primary defect in β-cells in many non-obese patients.

Methods

Participation

The Data Protection Authority of Iceland and the National Bioethics Committee of Iceland approved the study. All patients who participated in the study gave informed consent. All personal identifiers associated with blood samples, medical information, and genealogy were first encrypted by the Data Protection Authority, using a third-party encryption system (Gulcher and Kristjansson et al. 2000). This study was based on a list of 2400 Type II diabetic patients diagnosed either through a long-term epidemiologic study done at the Icelandic Heart Association over the past 30 years or at one of two major hospitals in Reykjavik over the past 12 years.

Two-thirds of these patients are alive, representing about half of the population of known Type II diabetes patients in Iceland. The majority of the patients have been contacted for this study, and the cooperation rate exceeded 90%. All participants in the Type II diabetes study visited the Icelandic Heart Association where each answered a questionnaire, had blood drawn and a fasting plasma glucose measurements taken. Questions about medication and age at diagnosis were included. Their height (m) and weight (kg) were measured to calculate BMI. The Type II diabetes patients in this study were diagnosed as described in our previously published linkage study (Reynisdottir and Thorleifsson et al. 2003). In brief, the diagnosis of Type II diabetes was confirmed by study physicians through previous medical records, medication history, and/or new laboratory measurements. For previously diagnosed Type II diabetes patients the reporting of oral glucose-lowering agent use confirmed Type II diabetes. Individuals who were currently treated with insulin were classified as having Type II diabetes if they were also using or had previously used oral glucose-lowering agents. In this cohort the majority of patients on medication take oral glucose-lowering agents and only a small portion (9%) require insulin. Although the prevalence of LADA (latent autoimmune diabetes in adults [MIM 222100, 138275]) among diabetic patients in Iceland is not formally known, it is generally considered to be less than 10% in European diabetics (Tuomi, Carlsson et al. 1999). For hitherto undiagnosed individuals, the diagnosis of Type II diabetes and impaired fasting glucose (IFG) was based on the criteria set by the American Diabetes Association (Expert Committee on the Diagnosis and Classification of Diabetes Mellitus 1997). The average age of the Type II diabetes patients in this study was 69.7 years.

Genotyping

New sequence repeats (i.e., dinucleotide, trinucleotide, and tetronucleotide repeats) were identified with the Sputnik program as found on the espresso software website and tested for polymorphicity in 94 controls. The size in basepairs of the lower allele of the CEPH sample 1347-02 (CEPH genomics repository) was subtracted from the size of the microsatellite amplicon and used as a reference.

Single nucleotide polymorphisms (SNPs) in the genes under investigation were detected by PCR sequencing exonic and intronic regions from 94 non-obese Type II diabetes patients and 94 population-based controls. Subsequently, the KChIP1 gene was sequenced in a total of 94 Icelandic patients and controls to identify additional SNPs for genotyping and to characterize the linkage disequilibrium structure of the region. Public polymorphisms were identified using the NCBI SNP database found on the the NCBI world wide website. Subsequent SNP genotyping was carried out using either fluorescent polarization template-directed dye-terminator incorporation (SNP-FP-TDI assay) or TaqMan (Applied Biosystems).

Insulin and GAD Antibody Measurements

Insulin and GAD antibody levels were measured in serum for the majority of the Type II diabetes patients according to the manufacturers' instructions. Insulin levels were measured using the AutoDELFIATM Insulin kit (Perkin Elmer). GAD antibody levels were quantified using the Diaplets Anti-GADPLUS ELISA assay (Roche Molecular Biochemicals). 94 LADA cases (GAD antibody<32 ng/ml) out of the 953 (9.86%) Type II diabetes cases were identified and tested.

Danish Cohort

The Danish study group was selected from the PERF (Prospective Epidemiological Risk Factors) study in Denmark (Bagger and Riis et al. 2001). Two hundred and eighty-two females had been diagnosed previously with Type II diabetes and/or measured >=7 mM glucose, of which, 149 were non-obese (BMI<30). As controls, 346 unaffected females were randomly drawn from the same study.

Statistical Methods for Association and Haplotype Analysis

For single marker association to Type II diabetes the fisher exact test was used to calculate a two-sided p-value for each allele. Allelic frequencies were presented rather than carrier frequencies for microsatellites, SNPs and haplotypes. To minimize any bias due to relatedness of the affected individuals, who were recruited as families for the linkage analysis, first- and second-degree relatives were eliminated from the list of affected individuals. For the haplotype analysis the program NEMO was used as described above. NEMO handles missing genotypes and uncertainty with phase through a likelihood procedure, using the expectation-maximization algorithm as a computational tool to estimate haplotype frequencies. Under the null hypotheses, the affected individuals and controls are assumed to have identical haplotype frequencies. Under the alternative hypotheses, the candidate at-risk haplotype is allowed to have a higher frequency in the affected individuals than in controls, while the ratios of frequencies of all other haplotypes are assumed to be the same in both groups. Likelihoods are maximized separately under both hypotheses, and a corresponding 1-degree-of freedom likelihood ratio statistics is used to evaluate the statistical significance.

To assess the significance of the haplotype association corrected for multiple testing across the candidate genes under investigation, a randomization test using the same genotype data was carried out. The cohorts of affected individuals and controls were randomized and the analysis was repeated. This procedure was repeated up to 1,000 times and the adjusted P value presented is the fraction of replications that produced a P value for a haplotype tested that is lower than or equal to the P value observed using the original patient and control cohorts.

For both single-marker and haplotype analysis, relative risk (RR) and population attributable risk (PAR) was calculated assuming a multiplicative model (Falk and Rubinstein, 1987; Terwilliger and Ott, 1992). LD between pairs of SNPs was calculated using the standard definition of D′ (Lewontin 1964) and R2 (Hill and Robertson 1968). Using NEMO, frequencies of the two marker allele combinations was estimated by maximum likelihood and evaluated deviation from linkage equilibrium by a likelihood ratio test. When plotting all SNP combinations to elucidate the LD structure in a particular region, D′ was plotted in the upper left corner and p-values in the lower right corner. In the LD plot, the markers are plotted equidistantly rather than according to their physical positions.

Western Blotting, Northern Blotting and Immunohistochemistry

For Western blotting, tissues were harvested from C57BL/6J mice and immediately frozen in liquid nitrogen. The frozen pancreas and the rat pancreatic β-cell line INS-1 (F. Hoffmann-La Roche, Basel) were lyzed in RIPA buffer (1% DOC, 1% NP-40, 0.1% SDS, 150 mM NaCl, 2 mM EDTA, 20 mM MOPS, pH 7.2 and protease inhibitor cocktail no. III, Calbiochem) for 30 minutes on ice. INS-1 cells from insulin secretion assays were lysed in Triton-X buffer (1% Triton-X, 150 mM NaCl, 1 mM EDTA and 10 mM Tris-HCl, pH 8.0). All lysates were cleared by centrifugation at 14 000 RPM for 20 minutes at 4° C. and the protein concentrations were determined (BCA, Pierce). A polyclonal antiserum against KChIP1 was produced because the commercial antibody used for the immunohistochemistry did not detect KChIP1 on a Western blot. A codon optimized recombinant KChIP1 protein (aa 34-216), with a C-terminal His tag, was expressed in Top10F′ cells (Invitrogen), purified on Ni-NTA superflow column and injected into rabbits. The antiserum was affinity purified before use. The specificity of the antiserum was determined by performing a competition experiment by probing an identical blot with the antiserum in the presence of an excess of the recombinant protein. As a loading control, the blots were re-probed with anti-GAPDH antibody (Research Diagnostics). The proteins were detected by ECL system (Amersham). A multiple-tissue Northern blot (human #7760-1; BD Bioscience) was hybridized with a human KChIP1 probe, spanning nucleotides 1-200 (exons 1 and 2). The probe was labeled with α-^{P}32-CTP by random primed DNA labeling (Roche) according to the manufacturer's instructions. The membrane was exposed to MS-film (Kodak) for 2 days. The probe detected only a single transcript suggesting that hybridization with the N-terminus of KChIP1 showed no cross-reactivity with other KChIP family mRNAs.

Retroviral Constructs

KChIP1 full-length human KChIP1 was cloned by PCR from Marathon ready cDNA (Clontech) into pENTR/D-TOPO (Invitrogen) and verified by sequencing. Subsequently, it was recombined with pDEST40 (Invitrogen) to create pDEST40-KChIP1, which is C-terminally tagged with V5 and His. KChIP1-V5 was cloned by PCR into the retroviral vector pQCXIN (BD-Bioscience) to create pQCXIN-KChIP1, a vector that expresses human KChIP1 tagged with V5 and His. As controls, we used the empty vector pQCXIN or a vector expressing GFP, pQCXIN-GFP.

For the knockout retrovirus construct, the pSIREN-RetroQ vector (BD Bioscience) was used and the target sequences were embedded in a hairpin oligonucleotide, according to the manufacturer's instructions. To select the target sequence, the siRNA-designer algorithm (BD Bioscience) was used and the selected sequences were examined for their specificity in targeting only KChIP1 (NCBI-Blast). The target sequence for construct #432 was AGAGGAGATGATGGACATT (SEQ. ID NO. 259). The oligonucleotides were annealed and ligated into the RNAi-Ready pSIREN-RetroQ vector according to the manufacturer's instructions (BD Biosciences). As a control, we employed either the luciferase sh-OligoDNA delivered with the vector (BD Bioscience) or pQCXIN-GFP (described above).

Overexpression and Knockout of KChIP1 in INS-1 Cells

Retroviruses were produced using the 293GPG cell line (generously provided by R. C. Mulligan, Harvard Medical School, Boston), which has an inducible expression of the VSV-G surface protein (tetracycline regulatory system) and a stable expression of the Gag and Pol viral packaging elements (Ory, Neugeboren et al. 1996). Stable producer cell lines expressing KChIP1, GFP, shLuciferase, shKChIP1#432 and an empty virus were generated as described by Ory et al., (Ory, Neugeboren et al. 1996). In brief, 293GPG cells were transfected with 20 μg of the different expression vectors and after 24 h the medium was replaced by tetracycline-free medium to induce VSV-G protein expression for packaging the virus. The supernatant was harvested and filtered 48 h later. To produce high titer virus stock used for INS-1 infection, the 293GPG cells were grown until 95% confluent and then tetracycline containing medium was removed. Virus containing supernatant was harvested over the next 24 h, 48 h and 72 h. The viral supernatant was filtered and concentrated by ultracentrifugation. The INS-1 cells were infected twice over a period of 4 days with the viral supernatant. Following the final infection, cells were split into selection medium containing 0.5 μg/ml puromycin or 200 μg/ml G418 for two weeks.

The level of KChIP1 knock-down was determined by Western blotting (described above) of 5, 10 and 25 ug of cell lysates. The Western blot was scanned and KODAK ID software was used to determine the ratio between the bands. To test for overexpression of protein, the cells were either immunofluorescently labeled with V5 mouse monoclonal antibody (data not shown) or by Western blotting (described above).

Insulin Secretion Assay

The INS-1 cells were trypsinized, resuspended in growth medium and washed three times in Krebs Ringer Buffer (KRB: 115 mM NaCl, 4.7 mM KCl, 2.56 mM CaCl₂, 1.2 mM KH2PO4, 1.2 mM MgSO4, 20 mM NaHCO3, 16 mM HEPES) containing 0.1% BSA and 0.5 mM glucose. Subsequently, the cells were resuspended in KRB and 150 000 cells were seeded in a 96 well plate (100 μl/well) that had been coated with Poly-D-Lysine. The cells were allowed to settle for 2 h at 37° C. before replacing the buffer with KRB containing 0.5 mM or 11.2 mM glucose and incubating for additional 2 h. The supernatant was collected, spun down, diluted 1:2 or 1:5 (FIGS. 10B and 11B respectively) and the secreted amount of insulin was quantified with a rat insulin ELISA kit (Mercodia). In order to have sufficient power for the analysis, 12 data points were measured for each condition. A student's t-test was applied assuming unequal variance to calculate the significance of the data (p-value: two sided) using Excel data analysis tools.

Correlation with Serum Insulin Levels

To determine if the S3 haplotype in KChIP1 showed a negative correlation to serum insulin levels as previously predicted (Reynisdottir and Thorleifsson et al. 2003) and in correlation with biological data presented above, a study cohort of 527 unrelated Type II diabetic patients, representing a large subset of those analyzed in the initial association study was used in this analysis. All patients had basal fasting insulin levels measured that were subsequently age and sex adjusted. The patients were GAD negative to exclude patients with LADA (latent autoimmune diabetes in adults, a form of Type 1 diabetes) and not on insulin treatment to exclude any confounding influence on the serum insulin measurements. For this cohort, a significant negative correlation was observed, with a reduction in age and sex-adjusted insulin levels by 0.100 per copy of haplotype S3 (one-sided p-value=0.045). Note that a one-sided p-value for this correlation is reported as postulated, based on previous linkage data and the reported assay work, that the correlation would be negative with respect to insulin levels.

Excluding all medicated Type II diabetes patients from the analysis, a significant negative correlation continued to be observed, with a greater reduction in age and sex-adjusted insulin levels of 0.124 per copy of haplotype S3 (one-sided p-value=0.075). Although the correlation was stronger in this group, the p-value is higher due to the fewer numbers analyzed in this set.

By splitting the two cohorts into upper and lower 50^{th} percentiles based on serum insulin levels, the frequency of the at-risk haplotype was greater in the lowest 50^{th} percentile for both cohorts (Table 19). Table 19 also highlights that the difference between the upper and lower 50^{th} percentiles was more apparent among the non-medicated set; in this case the frequency of haplotype S3 was significantly different between these defined groups (p-value=0.0046). Furthermore, homeostasis model assessment (HOMA), a measure of insulin sensitivity, can be determined by fasting insulin (μU dl−1)×fasting glucose (mg dl−1)/22.5. The lowest 50^{th} percentile of HOMA values can be considered as those non-medicated patients with normal insulin sensitivity as opposed to those in the upper 50^{th} percentile who can be considered as displaying insulin resistance. Haplotype S3 is significantly over-represented in the lower HOMA 50^{th} percentile (p=0.025).

These data are in keeping with the in vitro data and support the notion that KChIP1 may play a significant role in insulin-secretion.

In summary, this additional association study indicates two independent haplotypes, haplotypes S3 and D2, were observed carried by over 60% of non-obese Type II diabetes patients that confer risk to Type II diabetes in Iceland. It is shown that D2 also confers significant risk of Type II diabetes in Denmark. The study of the biology of KChIP1 in β-cells shows that this gene has a role in the regulation of insulin secretion in vitro. This appears to be true in vivo since this study finds that haplotype S3 correlates with lower insulin levels within the Type II diabetes patient cohort.

Discussion

As described herein, the positional cloning of a novel Type II diabetes gene, encoding KChIP1, within the 5q34-q35.2 locus is shown. This gene confers substantial risk of Type II diabetes in non-obese individuals (BMI<30). Biological data that point to a role for KChIP1 in the regulation of insulin secretion is also shown. KChIP1 is expressed in pancreatic β-cells and perturbations of KChIP1 expression in rat pancreatic β-cells affect basal and glucose stimulated insulin release. Serum insulin levels are decreased in patients with an at-risk haplotype, underscoring the role that KChIP1 variants may play in the pathogenesis of Type II diabetes. This is in line with the hypothesis from our previous linkage paper that a non-obese Type II diabetes study group is enriched with patients with primarily beta cell dysfunction as they do not need as severe a level of obesity related insulin resistance to unmask their diabetes. In fact, the data presented show that the KChIP1 gene variants inversely correlate with insulin resistance among Type II diabetes patients.

Two significant haplotypes in the KChIP1 gene that associate with Type II diabetes are described. The gene product is expressed in pancreatic β-cells and when its expression is reduced in an experimental system, more insulin is released. Conversely, overexpression of KChIP1 resulted in reduced insulin secretion. The same gene variants that show association to Type II diabetes also correlate with low insulin levels in Type II diabetes patients suggesting that these variants lead to increased KChIP1 and lower insulin secretion. Thus the genetic and functional data point to a control mechanism of insulin secretion.

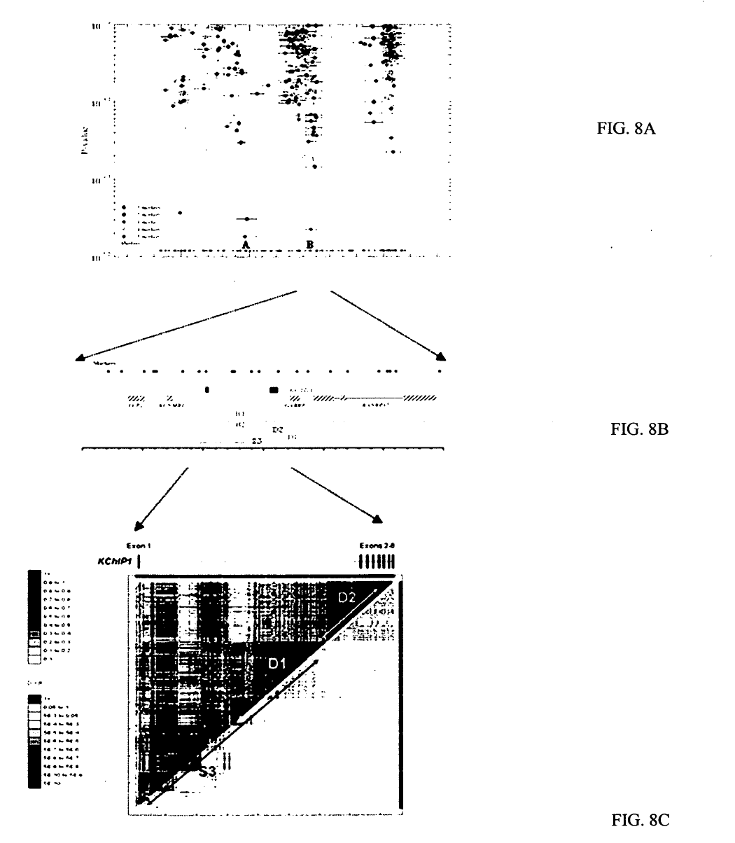

TABLE 16
Criteria for haplotype length different and
unrelated patients - restricted to 300 kb

| | | | Aff | | |
|---|---|---|---|---|---|
| Haplo- | | | allelic | Haplotype span | |
| type | p-value | RR | freq | (B34) | Markers in Haplotype |
| F1 | 3.1E−05 | 5.6 | 3.8 | 1.68.82-1.69.11 | −6 DG5S37 −14 DG5S40 |
| F2 | 1.8E−05 | 5.6 | 3.9 | 1.68.82-1.69.11 | −6 DG5S37 0 DG5S101 |
| | | | | | −14DG5S40 |
| G1 | 1.8E−05 | 9.8 | 5.3 | 169.75-170.00 | 0 DG5S46 6 DG5S47 |
| | | | | | 0 DG5S953 0 DG5S955 |
| G2 | 2.3E−05 | 8.9 | 5.4 | 169.83-170.00 | 6 DG5S47 0 DG5S953 |
| | | | | | 0 DG5S955 |
| Table 16: Haplotypes within Regions A and B of the 1-LOD-drop that show the strongest association to non-obese Type II diabetes. For each haplotype (i) a two-sided p-value for a single test of association to non-obese Type II diabetes patients, (ii) the corresponding relative risk (RR), (iii) the estimated allelic | | | | | |
| # frequency of the haplotype in the patient and the control cohort, (iv) the span of the haplotype (NCBI Build 34) and (v) the alleles (in bold) and markers that define the haplotype are shown. The relative location of haplotypes G1(shown as B1) and G2(shown as B2) are depicted in FIG. 8b. The control allelic frequencies of the A series and the B series haplotypes was 0.7% and 0.6% respectively. | | | | | |

TABLE 17
Table 17: Microsatellite and SNP haplotype association within KChIP1.
Type II Diabetes (T2D) Study

| | | | Aff allelic | |
|---|---|---|---|---|
| | p-value | RR | freq | Markers in haplotype |
| (a) | | | | |
| D1 haplotype | | | | |
| Non-obese | 1.00E−04 | Inf | 3.2% | −4 DG5S13 C SG05S176 0 D5S625 |
| T2D | | | | |
| Obese | 3.00E−03 | Inf | 2.6% | −4 DG5S13 C SG05S176 0 D5S625 |
| T2D | | | | |
| All T2D | 3.00E−03 | Inf | 2.6% | −4 DG5S13 C SG05S176 0 D5S625 |
| Danish | 0.06 | 0.0008 | 0.001% | −4 DG5S13 C SG05S176 0 D5S625 |
| T2D | | | | |
| (b) | | | | |
| D2 haplotype | | | | |
| Danish | 0.0004 | 2.59 | 10.5% | 0 DG5S124 C SG05S176 C SG05923 T |
| T2D | | | | SG05S187 A SG05S948 |
| All T2D | 0.025* | 1.52 | 8.2% | 0 DG5S124 C SG05S176 C SG05923 T |
| | | | | SG05S187 A SG05S948 |
| (a) The D1 haplotype is located in the 3′-end of the gene (170.0-170.15 Mb). Shown are results of a test of association for Type II diabetes patients vs. population controls for D1 in the Icelandic cohort (top) and a non-replication in a cohort of Danish Type | | | | |
| # II diabetes patients (bottom). The control allelic frequencies of D1 were undetectable in the Icelandic Type II diabetes cohort and 1.6% in the Danish Type II diabetes cohort. | | | | |
| (b) The most significant haplotype observed with the markers tested in the Danish Type II diabetes cohort, D2, was 54 kb long and had a relative risk of 2.59 and an aff allelic frequency of 10.5% (p = 0.0004). Interestingly, this haplotype was also observed in | | | | |
| # excess in the Icelandic Type II diabetes patient cohort, albeit with a lower relative risk of 1.52 (aff allelic frequency = 8.2%; one-sided p = 0.025). The control allelic frequencies of D1 in the Danish and Icelandic Type II diabetes cohorts were 4.3% and 5.5% respectively. | | | | |

*Note that we report a one-sided p-value for the test on the Icelandic cohort as that is a replication of association results previously observed in the Danish cohort.

TABLE 18
Table 18: SNP haplotype association across KChIP1. The most significant
SNP only haplotype observed in non-obese Type II diabetes was haplotype
S3 (p-value = 1.36 × 10⁻⁶; aff allelic freq = 59.8%;
RR = 1.61). The control allelic frequency of S3 was 48%

| | | | Aff allelic | |
|---|---|---|---|---|
| S3 | p-value | RR | freq | |
| Non-obese T2D | 1.36E−06 | 1.61 | 59.8% | G SG05S96 |
| | | | | C DG00AAJIB |
| | | | | C DG00AAJHF |
| Obese T2D | 2.99E−04 | 1.47 | 57.5% | G SG05S96 |
| | | | | C DG00AAJIB |
| | | | | C DG00AAJHF |
| All T2D | 4.95E−07 | 1.54 | 58.8% | G SG05S96 |
| | | | | C DG00AAJIB |
| | | | | C DG00AAJHF |

TABLE 19
Table 19: Haplotype S3 frequency in the upper and lower
50^{th} percentiles of sex-adjusted insulin levels among
Type II diabetes patients was identified.

| (a) TYPE II | | | | |
|---|---|---|---|---|
| DIABETES on | | | | |
| insulin | p-val | RR | Aff allelic freq | Con allelic freq |
| Lower 50^{th} | 9.6E−06 | 1.65 | 60% | 48% |
| percentile | | | | |
| Upper 50^{th} | 0.0076 | 1.35 | 56% | 48% |
| percentile | | | | |

| | | | Lower 50^{th} allelic | Upper 50^{th} allelic |
|---|---|---|---|---|
| | p-val | RR | freq | freq |
| Lower vs. Upper | 0.16 | 1.2 | 60% | 56% |
| percentile | | | | |

| (b) TYPE II | | | | |
|---|---|---|---|---|
| DIABETES not | | | | |
| on medication | p-val | RR | Aff allelic freq | Con allelic freq |
| Lower 50^{th} | 2.2E−05 | 1.84 | 63% | 48% |
| percentile | | | | |
| Upper 50^{th} | 0.47 | 1.11 | 52% | 48% |
| percentile | | | | |

| | | | Lower 50^{th} | Upper 50^{th} |
|---|---|---|---|---|
| | | | allelic | allelic |
| | p-val | RR | freq | freq |
| lower vs Upper. | 0.0046 | 1.67 | 63% | 52% |
| percentile | | | | |

(a) In a study cohort of GAD negative Type II diabetes patients not treated with insulin, the frequency of haplotype S3 was significantly higher in both the lowest and highest 50^{th} percentile of insulin; however the difference between the two groups was not significant (p = 0.16)
(b) For the subset of patients who were not on any diabetes medication, the frequency of haplotype S3 is significantly higher in the lowest 50^{th} percentile of insulin compared to the highest (p = 0.0046).

Markers in Candidates (Microsatellites in Italics)

TABLE 20
LCP 2: 5 microsatellites and 11 SNPs

| C05 Build 33 | Marker | deCODE alias |
|---|---|---|
| 169586308 | DG5S115 | |
| 169615490 | DG00AAIHM | LCP_47437 |
| 169615682 | DG00AAIHL | LCP_47245 |
| 169620730 | DG00AAIHK | LCP_42197 |
| 169625147 | DG00AAIHJ | LCP_37780 |
| 169625236 | DG00AAIHI | LCP_37691 |
| 169629401 | DG00AAIHH | LCP_33526 |
| 169633571 | DG00AAIHG | LCP_29356 |
| 169637933 | DG00AAIHF | LCP_24994 |
| 169638191 | DG00AAIHE | LCP_24736 |
| 169653708 | DG5S1173 | |
| 169655755 | DG00AAIHD | LCP_7173 |
| 169660310 | SG05S70 | LCP_2618 |
| 169661202 | DG5S44 | |
| 169693772 | DG5S45 | |
| 169702377 | DG5S46 | |

TABLE 21
KCNMB1: 7 microsatellites and 5 SNPs

| C05 Build 33 | Marker | Inhouse alias |
|---|---|---|
| 169653708 | DG5S1173 | |
| 169661202 | DG5S44 | |
| 169693772 | DG5S45 | |
| 169702377 | DG5S46 | |
| 169740666 | SG05S1611 | KNB_24222 |
| 169741173 | DG00AAIGG | KNB_24728 |
| 169745438 | DG5S1178 | |
| 169746339 | DG00AAIGH | KNB_29895 |
| 169751814 | DG00AAIHA | KNB_35370 |
| 169751843 | DG00AAIHB | KNB_35399 |
| 169788696 | DG5S47 | |
| 169843903 | DG5S119 | |

TABLE 22
KChIP1: 4 microsatellites and 6 SNPs-
positions shown in previous tables

| Marker | Inhouse alias |
|---|---|
| DG5S13 | |
| DG5S123 | |
| DG5S124 | |
| SG05S176 | KCP_1152 |
| SG05S923 | KCP_2649 |
| SG05S187 | KCP_4976 |
| SG05S179 | KCP_5077 |
| SG05S946 | KCP_15400 |
| SG05S948 | KCP_16152 |
| D5S625 | |

TABLE 23
GABRP: 8 microsatellites and 18 SNPs

| C05 Build 33 | Marker | Inhouse alias |
|---|---|---|
| 170015858 | DG5S123 | |
| 170041996 | DG5S124 | |
| 170105556 | D5S625 | |
| 170146165 | SG05S1753 | GAB_4360 |
| 170146304 | SG05S2147 | GAB_4499 |
| 170146363 | SG05S1754 | GAB_4558 |
| 170146503 | SG05S1755 | GAB_4698 |
| 170146540 | SG05S1756 | GAB_4735 |
| 170150984 | SG05S1757 | GAB_9179 |
| 170151033 | SG05S1758 | GAB_9228 |
| 170151181 | SG05S2148 | GAB_9376 |
| 170151190 | SG05S1759 | GAB_9385 |
| 170151213 | SG05S1760 | GAB_9408 |
| 170151321 | SG05S1761 | GAB_9516 |
| 170151417 | SG05S2246 | GAB_9558 |
| 170151703 | SG05S1762 | GAB_9898 |
| 170156610 | SG05S1750 | GAB_14805 |
| 170167429 | DG5S959 | |
| 170174367 | SG05S2144 | GAB_32642 |
| 170175717 | SG05S1751 | GAB_33912 |
| 170175966 | SG05S1752 | GAB_34161 |
| 170176037 | SG05S2146 | GAB_34232 |
| 170203240 | DG5S960 | |
| 170280782 | DG5S16 | |
| 170338421 | DG5S962 | |
| 170442700 | DG5S132 | |

TABLE 24
RANBP17: 11 microsatellites and 9 SNPs

| C05 Build 33 | Marker | Inhouse alias |
|---|---|---|
| 170203240 | DG5S960 | |
| 170220482 | SG05S2114 | RANRS1978461 |
| 170247896 | SG05S2111 | RANRS1423231 |
| 170280782 | DG5S16 | |
| 170315425 | SG05S2116 | RANRS477119 |
| 170338421 | DG5S962 | |
| 170406521 | SG05S2115 | RANRS2441016 |
| 170442700 | DG5S132 | |
| 170469573 | DG5S136 | |
| 170471385 | SG05S2113 | RANRS173465 |
| 170480360 | DG5S133 | |
| 170499980 | DG5S17 | |
| 170519072 | SG05S1613 | RANRS1445796 |
| 170635718 | SG05S1614 | RANRS2964525 |
| 170644993 | DG5S137 | |
| 170654830 | SG05S2112 | RANRS1469438 |
| 170673106 | DG5S53 | |
| 170675807 | DG5S968 | |
| 170681390 | SG05S2117 | RANRS891009 |
| 170735417 | DG5S904 | |

TABLE 25
66 SNPs selected-positions shown in previous table.

| Marker | Inhouse alias |
|---|---|
| SG05S83 | |
| SG05S96 | |
| DG00AAESP | |
| SG05S93 | |
| SG05S2107 | KCP_9677 |
| SG05S229 | KCP_10355 |
| DG00AAFCS | |
| DG00AAFCI | |
| DG00AAFCL | |
| SG05S556 | KCP_60703 |
| SG05S957 | KCP_82468 |
| SG05S64 | KCP_93545 |
| DG00AAJIB, | KCP_102882 |
| SG05S2479 | |
| DG00AAJIC | KCP_107380 |
| SG05S730 | KCP_113257 |
| SG05S745 | KCP_116355 |
| SG05S194 | KCP_124877 |
| SG05S195 | KCP_126086 |
| SG05S152 | KCP_129093 |
| SG05S480 | KCP_129127 |
| DG00AAJHB | KCP_130309 |
| SG05S197 | KCP_131244 |
| SG05S153 | KCP_131854 |
| SG05S154 | KCP_132677 |
| SG05S200 | KCP_135518 |
| SG05S481 | KCP_136036 |
| SG05S202 | KCP_137660 |
| SG05S482 | KCP_146746 |
| SG05S205 | KCP_149216 |
| DG00AAFCK | |
| SG05S208 | KCP_160145 |
| SG05S156 | KCP_169234 |
| SG05S210 | KCP_173848 |
| SG05S157 | KCP_173982 |
| DG00AAJHF | KCP_174243 |
| SG05S158 | KCP_175106 |
| DG00AAFCJ | |
| SG05S212 | KCP_180237 |
| SG05S213 | KCP_186048 |
| SG05S161 | KCP_186679 |
| DG00AAJDY | KCP_187116 |
| SG05S163 | KCP_193956 |
| DG00AAJEE | KCP_194616 |
| DG00AAJEF | KCP_196548 |
| DG00AAJEG | KCP_197678 |
| DG00AAJEH | KCP_197775 |
| DG00AAJEO | KCP_202795 |
| SG05S170 | KCP_207965 |
| SG05S186 | KCP_212775 |
| DG00AAFCF | |
| SG05S1285 | KCP_224153 |
| SG05S176 | KCP_224182 |
| SG05S923 | KCP_225679 |
| SG05S178 | KCP_227871 |
| SG05S483 | KCP_227987 |
| SG05S187 | KCP_228006 |
| SG05S1293 | KCP_232521 |
| SG05S188 | KCP_237505 |
| SG05S945 | KCP_238234 |
| SG05S946 | KCP_238430 |
| SG05S948 | KCP_239182 |
| SG05S189 | KCP_241099 |
| SG05S1608 | |
| SG05S1609 | |
| SG05S1610 | |
| DG00AADMS | |

KChIP1

The invention described herein has linked Type II diabetes to a gene encoding Kv channel-interacting protein 1 (KChIP1; also known as KCNIP1). In the brain and heart, rapidly inactivating (A-type) voltage-gated potassium (Kv) currents operate at subthreshold membrane potentials to control the excitability of neurons and cardiac myocytes. Although pore-forming alpha-subunits of the Kv4, or ShaI-related, channel family form A-Type currents in heterologous cells, these differ significantly from native A-Type currents. To identify proteins that interacted with the Kv4 subunit, An et al., (“Modulation of A-Type potassium channels by a family of calcium sensors” Nature 403:553-6 (2000)) used the yeast two-hybrid system with the intracellular amino terminus of the rat Kv4.3 subunit to screen rat midbrain cDNA libraries. Two Kv channel-interacting proteins were identified and called KChIPs (KChIP-1 and KChIP2). Library screening and database mining identified mouse and human orthologs of these genes. The KChIP1 cDNA encodes a 216-amino acid protein. The KChIPs have 4 EF-hand-like domains and bind calcium ions. Both KChIPs have distinct N termini but share approximately 70% amino acid identity throughout a carboxy-terminal 185-amino acid core domain that contains the 4 EF-hand-like motifs. Although the KChIPs have around 40% amino acid similarity to neuronal calcium sensor-1 and are members of the recovering/NCS subfamily of calcium-binding proteins, other members of this subfamily, such as hippocalcin, did not interact with Kv4 channels in the yeast 2-hybrid assay. An et al., (supra) additionally found that expression of KChIPs and Kv4 together reconstitutes several features of native A-Type currents by modulating the density, inactivation kinetics, and rate of recovery from inactivation of Kv4 channels in heterologous cells. Both KChIPs colocalize and coimmunoprecipitate with brain Kv4 alpha-subunits, and are thus integral components of native Kv4 channel complexes. As the activity and density of neuronal A-Type currents tightly control responses to excitatory synaptic inputs, these KChIPs may regulate A-Type currents, and hence neuronal excitability, in response to changes in intracellular calcium.

The glycosphingolipid sulfatide is present in secretory granules and at the surface of pancreatic β-cells (Buschard K, Fredman P. “Sulphatide as an antigen in diabetes mellitus”. Diabetes Nutr Metab 4:221-228 (1996)), and antisulfatide antibodies (ASA; IgG1) are found in serum from the majority of patients with newly diagnosed Type I diabetes. Buschard et al., (“Sulfatide controls insulin secretion by modulation of ATP-sensitive K(+)-channel activity and Ca(2+)-dependent exocytosis in rat pancreatic beta-cells” Diabetes 51:2514-21 (2002)) demonstrated that sulfatide produced a glucose- and concentration-dependent inhibition of insulin release from isolated rat pancreatic islets. This inhibition of insulin secretion was due to activation of ATP-sensitive K⁺-(K_{ATP}) channels in single rat β-cells. No effect of sulfatide was observed on whole-cell Ca²⁺-channel activity or glucose-induced elevation of cytoplasmic Ca²⁺ concentration. A key observation was that sulfatide stimulated Ca²⁺-dependent exocytosis determined by capacitance measurements and depolarized-induced insulin secretion from islets exposed to diazoxide and high external KCl. The monoclonal sulfatide antibody Sulph I as well as ASA-positive serum reduced glucose-induced insulin secretion by inhibition of Ca²⁺-dependent exocytosis. This suggests that sulfatide is important for the control of glucose-induced insulin secretion and that both an increase and a decrease in the sulfatide content have an impact on the secretory capacity of the individual β-cells.

In pancreatic β-cells, glucose triggers the closing of ATP dependent potassium channels resulting in depolarization of the membrane. This subsequently opens voltage-dependent Ca²⁺ channels, allowing an influx of Ca²⁺ leading to Ca²⁺ stimulated vesicle fusion and secretion of insulin. Membrane repolarization is mediated by the activation of voltage dependent K⁺ (Kv) channels in β-cells (Philipson, Rosenberg et al., 1994; MacDonald, Ha et al., 2001; MacDonald and Wheeler 2003). It has been suggested that in CNS neurons, KChIP1 plays a role in Kv channel trafficking and release from the ER (O'Callaghan, Hasdemir et al., 2003; Shibata, Misonou et al., 2003). KChIP1 may have a chaperone function that increases either surface expression or specific activity of Kv channels in pancreatic β-cells and thus, leads to a faster repolarization of the membrane and ultimately in decreased insulin secretion (Philipson, Rosenberg et al., 1994; MacDonald, Ha et al., 2001; MacDonald and Wheeler 2003). Thus, knock-down of KChIP1 may alter β-cell membrane repolarization kinetics, perhaps extending the period of membrane depolarization and increasing insulin secretion. Alternatively, KChIP1 may directly regulate trafficking of insulin vesicles exiting the post-ER, perhaps acting as a negative regulator of trafficking of insulin vesicles or release of insulin.

Type II diabetes is characterized by hyperglycemia, which occurs when insulin secretion from the pancreatic β-cells can no longer compensate for the insulin resistance of the target tissues (Dornhorst and Merrin 1994). However, it has been debated whether insulin resistance or impaired β-cell function is the primary defect resulting in Type II diabetes (Gerich, Diabetes 51 Suppl 1: S117-21 (2002).

Identification of At-Risk Variants Through Resequencing

The most specific haplotype observed, i.e. S7, was elected to carry out a resequencing exercise to identify more specific at-risk variants enriched on the haplotype background such that they are closer to the causative variant in this complex region of LD and, therefore, more likely to replicate in additional T2D cohorts.

The same primer pairs employed for SNP discovery across the gene were used in this approach. In parallel to the knowledge, gained from the previous gene sequencing effort, an ‘equivalence’ class of 9 SNPs was observed that was enriched among T2D patients carrying the S7 haplotype compared to the control carriers, and near absent in the non-carriers. These SNPs —SG05S808, SG05S792, SG05S1234, SG05S1237, SG05S814, SG05S1239, SG05S820, SG05S824 and SG05S1242. were perfect surrogates to each other and were all located in intron 1 of the KChIP1 gene.

One of these SNPs, SG05S808, was selected for further genotyping in the 802 T2D patients and 570 population based controls. The results show that there was excess of the minor T allele for this SNP both in non-obese (RR=1.44; p=0.026) and obese (RR=1.36; p=0.087, albeit of borderline significance) T2D patients. We sought to analyze if the association of this variant to T2D could be replicated in additional T2D cohorts.

Replication of the SG05S808 association to T2D

To verify the association of the minor T allele of the SG05S808 SNP to T2D, the SNP was genotpyed in a second Icelandic T2D cohort and in a T2D cohort from the US (Pennsylvania). As outlined in the table below, the association of the minor T allele of SG05S808 to obese T2D replicated in 2 additional cohorts i.e. in the second cohort of Icelandic T2D (RR=1.48; p=0.035), and in the US Caucasian obese T2D (RR=2.1; p=0.009). The original observation in non-obese T2D patients was not replicated in any of the additional cohorts.

| | two-sided | | | Aff | | Ctrl |
|---|---|---|---|---|---|---|
| ORIGINAL STUDY GROUP | p-value | RR | #aff | freq | #ctrl | freq |
| FIRST ICELANDIC: | 0.026 | 1.443 | 428 | 0.100 | 529 | 0.072 |
| Non-obese T2D | | | | | | |
| FIRST ICELANDIC: | 0.087 | 1.361 | 341 | 0.095 | 529 | 0.072 |
| Obese T2D | | | | | | |
| REPLICATION | one-sided | | | Aff | | Ctrl |
| STUDY GROUP | p-value | RR | #aff | freq | #ctrl | freq |
| SECOND ICELANDIC: | NS | 1.048 | 240 | 0.079 | 508 | 0.076 |
| Non-obese T2D | | | | | | |
| SECOND ICELANDIC: | 0.035 | 1.479 | 171 | 0.108 | 508 | 0.076 |
| Obese T2D | | | | | | |
| USA Caucasian: | NS | 0.976 | 136 | 0.070 | 168 | 0.071 |
| Non-obese T2D | | | | | | |
| USA Caucasian: | 0.009 | 2.097 | 90 | 0.139 | 168 | 0.071 |
| Obese T2D | | | | | | |

SG05S808 belongs to class of SNPs that are perfect surrogates to each other.

| SNP | B34 location |
|---|---|
| SG05S792 | 170048790 |
| SG05S1234 | 170052262 |
| SG05S1237 | 170055870 |
| SG05S808 | 170059078 |
| SG05S814 | 170061607 |
| SG05S1239 | 170062143 |
| SG05S820 | 170063925 |
| SG05S824 | 170065310 |
| SG05S1242 | 170066796 |

Correlation with Serum Insulin Levels

The demonstration of a role for KChIP1 in insulin secretion led us to determine if the T-allele of the SNP SG05S808, shown above to be associated with T2D in Iceland and the US, showed a negative correlation to serum insulin levels in T2D cases. A study cohort of 747 T2D patients, representing a large subset of those analyzed in the initial association study was used in this analysis. All had basal fasting insulin levels measured that were subsequently log-transformed. The patients were GAD negative to exclude patients with LADA (latent autoimmune diabetes in adults, a form of Type I diabetes) and not on any insulin treatment to exclude any confounding influence on the serum insulin measurements. For the obese T2D cohort (n=326), a significant negative correlation was observed, with a reduction in log-transformed serum insulin levels with increasing copy number of the SG05S808 at-risk T allele (one-sided p-value=0.02). Note that we report a one-sided p-value for this correlation as we postulated that the correlation would be negative with respect to insulin levels.

Excluding medicated T2D patients from the analysis, a border-line significant negative correlation continued to be observed in 153 obese T2D cases, with increasing copy number of the SG05S808 at-risk T allele (one-sided p-value=0.055)—the p-value is higher due to the fewer numbers analyzed in this set.

TABLE 26
SURROGATE SNPs IDENTIFIED BY ADDITIONAL RESEQUENCING

| | | SEQ | | |
|---|---|---|---|---|
| Build 33 | DECODE | PROJECT | | |
| Pos | ALIAS | ALIAS | AMPLIMER SEQUENCE LISTING | SNP |
| 1700034438 | SG05S792 | KCP_146328 | GTGTGTGCCAGGCATGTTCATGTATA | A/G |
| | | | TTCAGGAAGAAGTGTCAGTATTTAAG | |
| | | | ATCCTCGGCCCTTGCCCGAGTCCCCA | |
| | | | ACACGCCTTCTTGTCTGGAGAACTGT | |
| | | | AAATCTTGGAAACATCTTGCAAGGG | |
| | | | GGGACACCTCACAGAAGGCAGGCTT | |
| | | | GGCATGGGATAAACAGAATCGACTC | |
| | | | CTCTGCTTCCTTCTGATGCAC[A/G]GT | |
| | | | GAATGGGCAGGTGGAAGCATCGTTG | |
| | | | CTTAAAGAGGAACCAAAACTCCACC | |
| | | | CCAGAGCTGCTAATTCCTTTTGGCTT | |
| | | | GCAGTTATGCAGAGGGCTAAAAAAT | |
| | | | CCAACGAATCACAAATCCCCTGGTTG | |
| | | | CTAAGTAGAAAGAATATGTTTTGGCT | |
| | | | GCTGCTGTTCCCTTCCCCAAGGAAAA | |
| | | | GATTCAAGCAGAGGCGGTC | |
| | | | (SEQ ID NO: 666) | |
| 170006910 | SG05S1234 | KCP_149800 | CTTCAAGTGCCACCTTCATCCCATTC | A/G |
| | | | TTTCTGCAAATATTCACCACACACCT | |
| | | | ACGTGACCTCAGGCTCTGTGTCAGGT | |
| | | | CCTGGGGATGTAATGGTGTCCATGAA | |
| | | | GAAACAAGGTCCCTGCCCTCATAGA | |
| | | | GTGGCCTGACATATGCCCGAG | |
| | | | GCAGTCAGCAGCCGAGTGCGGGAGA | |
| | | | CTCTTGAGCAGAGATTGAGTGTGTT[A/ | |
| | | | G]ATATCTGTAGGCATCAGCCTGGC | |
| | | | TTTGCTGAGTGAGCTATATCAGAGTG | |
| | | | GAGGAGGCCAGAGGCAAAGTCCAGA | |
| | | | CTCCACTGGATCCTGGATTGAGGGGA | |
| | | | GAAGGGGCTGGGCGGAGGAGCAGCC | |
| | | | TGAGCACCTGCATCTCACTCCAACTG | |
| | | | GGTGCTGATTTGTCCCCATGGCCCCA | |
| | | | GCACCCAGGCAGGTCACCAAGTA | |
| | | | (SEQ ID NO: 677) | |
| 170010518 | SG05S1237 | KCP_153408 | AGAAGTTGGAATTCATTAAAATTTAA | C/T |
| | | | ACCTCTGCCCTGTAAAAGGCAATGTT | |
| | | | AAGAGAATGAGAAAATAAGCCACAG | |
| | | | ACTGGGAAAAAATATTTGAAAAAAT | |
| | | | ATATATCTGATAAAGGACTGTTATTC | |
| | | | AAAGCATACAAAGAACTCTTAAAAA | |
| | | | TCAATAATTAGAAAATTAAAAATCTG | |
| | | | ATTCAAAAATAAGCAGAAGAC[C/T]C | |
| | | | GAACAGACATTTCACCAAGGAAGAT | |
| | | | ATACAGATGGCAAGTAAGCATGTGA | |
| | | | AAAAATGCTCCACATATGTCATTAGG | |
| | | | GAATTGCTAATTAAAACAATGATGA | |
| | | | GATGCCACTACAGAACTCATAGAAT | |
| | | | GGCCAATATCCAAAACACTTAACAA | |
| | | | CACAAAATGTAGGTGAGAATGTGGA | |
| | | | GAAACAGGAACTCTCATCAGTGC | |
| | | | (SEQ ID NO: 689) | |
| 170013726 | SG05S808 | KCP_156616 | CACATTCTAATTAGGAGCTTCTGAAC | C/T |
| | | | CCAAAGGAATTTCAGATAAGGGGAA | |
| | | | ATTTAGGCCCAAAGCCAGGAGAAGG | |
| | | | GGTGAGTAGGGCTTGATCTCTGCCTC | |
| | | | TGAAGGGCAGAGGGCGTGGACTATT | |
| | | | CTTGGCTCTTAGGGGACAGCTAGAG | |
| | | | AAATGTGGGTCTCATGGCGACAACTC | |
| | | | TGGACTCCATTGGAAGAACCTT[C/T]T | |
| | | | AACAGTCAGGGCTCCCAGAGATAAC | |
| | | | TAGACAAGTCACCAAGAGAGGCAGT | |
| | | | GGGTACCCCTCACAGGAGGGGTGCA | |
| | | | AATCAAAGCCAAGGCTTGGAGTGGA | |
| | | | CCATATTAAATCCATTTCTTATCCTGT | |
| | | | GATTCTTAGAGTCCTATCTGTATCAG | |
| | | | GGGAAGGCAGGTGGGTTCTAGAACT | |
| | | | TTCTAAATGTGTCCCTGTGG | |
| | | | (SEQ ID NO: 696) | |
| 170016255 | SG05S814 | KCP_159145 | GGCTACAAATGAATGTAAAAATCTCT | A/G |
| | | | AATTTAGTGCCAAGTAACAGAAAAC | |
| | | | AGCTCTACTTATCTTAAGCCAAAAAG | |
| | | | AGGGACTTCTCAGAGGCATACTAAT | |
| | | | GGAGGATGGCAAGAGGGCCTCACGT | |
| | | | GGAACCAGGGCCTGGAGCGGCACAG | |
| | | | CATTCAGGAAGCTCAGTCTCCTTCTC | |
| | | | TCTCTCTCTCTCATCTCTGCTT[A/G]TT | |
| | | | TCCCTTTACCTGTAGACATGCTATCA | |
| | | | TTTTTCCAATATCCATGGCAGAATGT | |
| | | | GGCCACCAGTAACTCCAGGTGTATA | |
| | | | ACAGAAGCCTGGCCTC | |
| | | | CCAGGAGAGGGTGACTTGATGCCCTT | |
| | | | AGCTCCAGCCTTCCACTCCCACCCTG | |
| | | | GGCCAAGCAACTGGTGGCCAGGAGG | |
| | | | AGCTCATATTGCAGGAATATTGAAAT | |
| | | | TT | |
| | | | (SEQ ID NO: 704) | |
| 170016791 | SG05S1239 | KCP_159681 | TGCACCAGTGCCCAAGTCTGTCACTC | A/G |
| | | | CCTCCTCTCATTCTGAAACATCCTCT | |
| | | | GCACTCCATTCACCAGTGCCAAGCCT | |
| | | | TGCCCTTCTTCAGGGTTCAACTTTGT | |
| | | | CCCACCTTCTCTGAGAAGGCTGCCCT | |
| | | | GGTCCCTGCAACCGCTATGA | |
| | | | GCTGCCTTTTCTGAGCTCTTCTGCTGT | |
| | | | GGAGGATCCATGCCATTGACCTA[A/G] | |
| | | | GCACCCGTTTTCCACATATTGAGCAT | |
| | | | TGCTGAGCACCTATTCTGTGCCAGGC | |
| | | | ACTGTGCTTCAGGGCCATGGGGGAT | |
| | | | GCTCCAAGCGGTAAAATG | |
| | | | CAACCAAAGCCCCGAAGGAGCTCAC | |
| | | | ATTCTAGTCATGTCCACAAAGAGGTA | |
| | | | ATAAATCCATAAATTGTATGTACTAT | |
| | | | TCTAGTCACAATAAAATTGTGTCGTA | |
| | | | CT | |
| | | | (SEQ ID NO: 706) | |
| 170018573 | SG05S820 | KCP_161463 | AAGAAACCAGCCTCTCTCAAATCAAT | C/T |
| | | | CAGTCATCTTGCTCATTGACCAATGG | |
| | | | TTCGGCCACCCGGCAAGAAATTAGA | |
| | | | AAAATCTATGCATAGGAGAAAGCTT | |
| | | | TCCAACAGCCCTGGAAAGGCCTTGG | |
| | | | GCCCCAAAGGGCTAGTATGAGGTTG | |
| | | | AGGTGGGGGAGAGCAAGAGAGGAC | |
| | | | AAAACAGGCTGGGTACTGACCTTC[C/ | |
| | | | T]TCGTACTCCATGGCTCAGAACTCTG | |
| | | | ACCTCTTGCCAACCTGCCTTCCGCAA | |
| | | | GCAGCGAGAGTAGCACGGCTCTGAG | |
| | | | GGGCATGGCCAGGGAGGATGTGACC | |
| | | | TCTAAGTGCTCCAAGTCACTGGGAAC | |
| | | | CCTGTTCTAATAATAGGCAAAGCTTC | |
| | | | AGTTGATGGAGTGCCTATATGGGCCC | |
| | | | AGCCCTGTGAACAGCTTCCTC | |
| | | | (SEQ ID NO: 713) | |
| 170019958 | SG05S824 | KCP_162848 | GTCTTGAACTCCTGACCTCAGGTGAT | C/G |
| | | | CTGAGCACCTCGATCTCCCAAAGTCC | |
| | | | TGGGATTACAGGTGTGAGTCACCATG | |
| | | | CCCAGCCAACTCAGTAAGTCTTTTGA | |
| | | | ATGTGAGCGGAGGCCCCCAGTCAGC | |
| | | | ATTTCCAAGACCTGGGAAATG | |
| | | | AGGGTGAGGAGGGGGCCAAGAGCTC | |
| | | | TGATTTCAAGTCCCAGCCCTGCCAC | |
| | | | [C/G]CATTCCTTAGGGCAGGCACCCAC | |
| | | | CACCGCCTTGGCTCAGGCCTCAGTTT | |
| | | | CCATCTGGAAGGTGCTGGCTGCCTTG | |
| | | | ATGGGCCTCTATGGGCCCTC | |
| | | | CAGCTCTGTGGCTCCATGTCAAATGC | |
| | | | CCCTGACCACCCTTGAGATTAACCCT | |
| | | | TTCCCAAGGATAGACAGCTTCCCCCA | |
| | | | TCATTCTAGATCCCTCACACTTCCCC | |
| | | | A | |
| | | | (SEQ ID NO: 267) | |
| 170021444 | SG05S1242 | KCP_164334 | GCATCCAGGCCACAGCAAAAGCATG | A/G |
| | | | AGACATACTTGGTGAGTGAGCAAGA | |
| | | | GAATGGAAGAAGAAATCAATGGAGC | |
| | | | CAAATGACTCCACCAGATTTGCATTT | |
| | | | GAGACGGTCACTGCAAGACTGCACC | |
| | | | AGTCCCAAATTCCCAAGACAGCAAT | |
| | | | GTTTTCCACGATGGGTCTGAACATGT | |
| | | | AACCATTCTCAGGTTCCTGTTGG[A/G] | |
| | | | GGTTTGGGAGCCTAGAAACTGAGAC | |
| | | | AGCCTCACAGCTGACAGATCACGAG | |
| | | | CAGCCATCTAAGTCGACGTTTCCAAA | |
| | | | GTCATGGGGCCTATGGTTTGTGAGCT | |
| | | | TATTTAGGTTGTCCCCGGGCCCAGCA | |
| | | | TCAAAAGCATTGAGACACGTACTGA | |
| | | | GGGACTCTTTTCCTAGCCTCTCAGTC | |
| | | | CTGACTGCTCAAGGACCAAGT | |
| | | | (SEQ ID NO: 718) | |

### REFERENCES

Altshuler, D., J. N. Hirschhorn, et al. (2000). “The common PPARgamma Pro12Ala polymorphism is associated with decreased risk of type 2 diabetes.” Nat. Genet., 26(1): 76-80.
Amos, A. F., D. J. McCarty, et al. (1997). “The rising global burden of diabetes and its complications: estimates and projections to the year 2010.” Diabet. Med., 14 Suppl 5: S1-85.
An, W. F., M. R. Bowlby, et al. (2000). “Modulation of A-type potassium channels by a family of calcium sensors.” Nature, 403(6769): 553-6.
Bagger, Y. Z., B. J. Riis, et al. (2001). “Risk factors for development of osteoporosis and cardiovascular disease in postmenopausal Danish women: The PERF Study.” J Bone Miner. Res. Suppl. 1: 1-396.
Barnett, A. H., C. Eff, et al., (1981). “Diabetes in identical twins. A study of 200 pairs.” Diabetologia 20(2): 87-93.
Beck, E. J., M. Bowlby, et al., (2002). “Remodelling inactivation gating of Kv4 channels by KChIP1, a small-molecular-weight calcium-binding protein.” J Physiol 538(Pt 3): 691-706.
Chang, L. S., C. Y. Chen, et al., (2003). “Functional implication with the metal-binding properties of KChIP1.” Biochem Biophys Res Commun 311(2): 258-63.
Dornhorst, A. and P. K. Merrin (1994). “Primary, secondary and tertiary prevention of non-insulin-dependent diabetes.” Postgrad. Med. J., 70(826): 529-35.
Falk, C. T. and P. Rubinstein (1987). “Haplotype relative risks: an easy reliable way to construct a proper control sample for risk calculations.” Ann. Hum. Genet., 51 (Pt 3): 227-33.
Gerich, J. E. (2002). “Is reduced first-phase insulin release the earliest detectable abnormality in individuals destined to develop type 2 diabetes?” Diabetes, 51 Suppl 1: S117-21.
Gloyn, A. L. (2003). “The search for type 2 diabetes genes.” Ageing Res Rev 2(2): 111-27.
Gloyn, A. L., M. N. Weedon, et al. (2003). “Large-scale association studies of variants in genes encoding the pancreatic beta-cell KATP channel subunits Kir6.2 (KCNJ11) and SUR1 (ABCC8) confirm that the KCNJ11 E23K variant is associated with type 2 diabetes.” Diabetes, 52(2): 568-72.
Gretarsdottir, S., G. Thorleifsson, et al. (2003). “The gene encoding phosphodiesterase 4D confers risk of ischemic stroke.” Nat. Genet. 35(2): 131-8.
Gulcher, J. R., K. Kristjansson, et al. (2000). “Protection of privacy by third-party encryption in genetic research in Iceland.” Eur. J. Hum. Genet. 8(10): 739-42.
Hatano, N., S. Ohya, et al. (2002). “Functional interaction between KChIP1 and GFP-fused Kv4.3L co-expressed in HEK293 cells.” Pflugers Arch 444(1-2): 80-8.
Hill, W. G. and A. Robertson (1968). “The effects of inbreeding at loci with heterozygote advantage.” Genetics, 60(3): 615-28.
Horikawa, Y., N. Oda, et al. (2000). “Genetic variation in the gene encoding calpain-10 is associated with type 2 diabetes mellitus.” Nat. Genet. 26(2): 163-75.
Knowler, W. C., D. J. Pettitt, et al. (1990). “Diabetes mellitus in the Pima Indians: incidence, risk factors and pathogenesis.” Diabetes Metab. Rev. 6(1): 1-27.
Lewontin, R. C. (1964). “The Interaction of Selection and Linkage. Ii. Optimum Models.” Genetics 50: 757-82.
MacDonald, P. E., X. F. Ha, et al. (2001). “Members of the Kv1 and Kv2 voltage-dependent K(+) channel families regulate insulin secretion.” Mol. Endocrinol. 15(8): 1423-35.
MacDonald, P. E. and M. B. Wheeler (2003). “Voltage-dependent K(+) channels in pancreatic beta cells: role, regulation and potential as therapeutic targets.” Diabetologia 46(8): 1046-62.
Newman, B., J. V. Selby, et al. (1987). “Concordance for type 2 (non-insulin-dependent) diabetes mellitus in male twins.” Diabetologia 30(10): 763-8.
O'Callaghan, D. W., B. Hasdemir, et al. (2003). “Residues within the myristoylation motif determine intracellular targeting of the neuronal Ca2+ sensor protein KChIP1 to post-ER transport vesicles and traffic of Kv4 K⁺ channels.” J. Cell. Sci. 116(Pt 23): 4833-45.
Ory, D. S., B. A. Neugeboren, et al. (1996). “A stable human-derived packaging cell line for production of high titer retrovirus/vesicular stomatitis virus G pseudotypes.” Proc. Natl. Acad. Sci. USA 93(21): 11400-6.
Philipson, L. H., M. P. Rosenberg, et al. (1994). “Delayed rectifier K+ channel overexpression in transgenic islets and beta-cells associated with impaired glucose responsiveness.” J. Biol. Chem., 269(45): 27787-90.
Reynisdottir, I., G. Thorleifsson, et al. (2003). “Localization of a susceptibility gene for type 2 diabetes to chromosome 5q34-q35.2.” Am. J. Hum. Genet. 73(2): 323-35.
Reynisdottir, I., G. Thorleifsson, et al. (2003). “Localization of a susceptibility gene for type 2 diabetes to chromosome 5q34-q35.2.” Am. J. Hum. Genet. 73(2): 323-35.
Shibata, R., H. Misonou, et al. (2003). “A fundamental role for KChIPs in determining the molecular properties and trafficking of Kv4.2 potassium channels.” J. Biol. Chem., 278(38): 36445-54.
Terwilliger, J. D. and J. Ott (1992). “A haplotype-based ‘haplotype relative risk’ approach to detecting allelic associations.” 42(6): 337-46.
Tuomi, T., A. Carlsson, et al. (1999). “Clinical and genetic characteristics of type 2 diabetes with and without GAD antibodies.” Diabetes, 48(1): 150-7.
Zimmet, P., R. Taylor, et al. (1983). “Prevalence of diabetes and impaired glucose tolerance in the biracial (Melanesian and Indian) population of Fiji: a rural-urban comparison.” Am. J. Epidemiol., 118(5): 673-88.

The teachings of all publications cited herein are incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

1. A method of diagnosing a susceptibility to Type II diabetes in an individual, comprising detecting a polymorphism in a KChIP1 nucleic acid, wherein the presence of the polymorphism in the nucleic acid is indicative of a susceptibility to Type II diabetes.

2. A method of diagnosing a susceptibility to Type II diabetes comprising detecting an alteration in the expression or composition of a polypeptide encoded by KChIP1 nucleic acid in a test sample, in comparison with the expression or composition of a polypeptide encoded by a KChIP1 nucleic acid in a control sample, wherein the presence of an alteration in expression or composition of the polypeptide in the test sample is indicative of a susceptibility to Type II diabetes.

3. The method of claim 1, wherein the polymorphism in the KChIP1 nucleic acid is indicated by detecting the presence of a least one of the polymorphisms indicated in Table 13.

4. The method of claim 1, wherein the polymorphism in the KChIP1 nucleic acid is indicated by detecting the presence of a least one of the polymorphisms indicated in Table 26.

5. An isolated nucleic acid molecule comprising a KChIP1 nucleic acid, wherein the KChIP1 nucleic acid has a nucleotide sequence selected from the group of nucleic acid sequences as shown in Table 10, or the complements of the group of nucleic acid sequences as shown in Table 10, wherein the nucleotide sequence contains a polymorphism.

6. An isolated nucleic acid molecule which hybridizes under high stringency conditions to a nucleotide sequence selected from the group of nucleic acid sequences as shown in Table 10, or the complements of the group of nucleic acid sequences as shown in Table 10, wherein the nucleotide sequence contains a polymorphism.

7. A method for assaying for the presence of a first nucleic acid molecule in a sample, comprising contacting said sample with a second nucleic acid molecule, where the second nucleic acid molecule comprises a nucleotide sequence selected from the group consisting of: nucleic acid sequences as shown in Table 10 and the complement of the nucleic acid sequences as shown in Table 10, wherein the nucleotide sequence contains a polymorphism and hybridizes to the first nucleic acid under high stringency conditions.

8. A vector comprising an isolated nucleic acid molecule selected from the group consisting of:
a) nucleic acid sequences as shown in Table 10; and
b) complement of one of the nucleic acid sequences are shown in Table 10; and
wherein the nucleic acid molecule contains a polymorphism and is operably linked to a regulatory sequence.

9. A recombinant host cell comprising the vector of claim 7.

10. A method for producing a polypeptide encoded by an isolated nucleic acid molecule having a polymorphism, comprising culturing the recombinant host cell of claim 8 under conditions suitable for expression of the nucleic acid molecule.

11. A method of assaying for the presence of a polypeptide encoded by an isolated nucleic acid molecule according to claim 5 in a sample, the method comprising contacting the sample with an antibody which specifically binds to the encoded polypeptide.

12. A method of identifying an agent that alters expression of a KChIP1 nucleic acid, comprising:
a) contacting a solution containing a nucleic acid comprising the promoter region of the KChIP1 nucleic acid operably linked to a reporter gene with an agent to be tested;
b) assessing the level of expression of the reporter gene; and
c) comparing the level of expression with a level of expression of the reporter gene in the absence of the agent; wherein if the level of expression of the reporter gene in the presence of the agent differs, by an amount that is statistically significant, from the level of expression in the absence of the agent, then the agent is an agent that alters expression of the KChIP1 nucleic acid.

13. An agent that alters expression of the KChIP1 nucleic acid, identifiable according to the method of claim 12.

14. A method of identifying an agent that alters expression of a KChIP1 nucleic acid, comprising:
a) contacting a solution containing a nucleic acid of claim 1 or a derivative or fragment thereof with an agent to be tested;
b) comparing expression with expression of the nucleic acid, derivative or fragment in the absence of the agent;
wherein if expression of the nucleotide, derivative or fragment in the presence of the agent differs, by an amount that is statistically significant, from the expression in the absence of the agent, then the agent is an agent that alters expression of the KChIP1 nucleic acid.

15. The method of claim 14, wherein the expression of the nucleotide, derivative or fragment in the presence of the agent comprises expression of one or more splicing variant(s) that differ in kind or in quantity from the expression of one or more splicing variant(s) the absence of the agent.

16. An agent that alters expression of a KChIP1 nucleic acid, identifiable according to the method of claim 15.

17. An agent that alters expression of a KChIP1 nucleic acid, selected from the group consisting of: antisense nucleic acid to a KChIP1 nucleic acid; a KChIP1 polypeptide; a KChIP1 nucleic acid receptor; a KChIP1 binding agent; a peptidomimetic; a fusion protein; a prodrug thereof; an antibody; and a ribozyme.

18. A method of altering expression of a KChIP1 nucleic acid, comprising contacting a cell containing a KChIP1 nucleic acid with an agent of claim 17.

19. A method of identifying a polypeptide which interacts with a KChIP1 polypeptide comprising a polymorphism indicated in Table 13 or Table 26, comprising employing a yeast two-hybrid system using a first vector which comprises a nucleic acid encoding a DNA binding domain and a KChIP1 polypeptide, splicing variant, or a fragment or derivative thereof, and a second vector which comprises a nucleic acid encoding a transcription activation domain and a nucleic acid encoding a test polypeptide, wherein if transcriptional activation occurs in the yeast two-hybrid system, the test polypeptide is a polypeptide which interacts with a KChIP1 polypeptide.

20. A Type II diabetes therapeutic agent selected from the group consisting of: a KChIP1 nucleic acid or fragment or derivative thereof; a polypeptide encoded by a KChIP1 nucleic acid; a KChIP1 receptor; a KChIP1 nucleic acid binding agent; a peptidomimetic; a fusion protein; a prodrug; an antibody; an agent that alters KChIP1 nucleic acid expression; an agent that alters activity of a polypeptide encoded by a KChIP1 nucleic acid; an agent that alters posttranscriptional processing of a polypeptide encoded by a KChIP1 nucleic acid; an agent that alters interaction of a KChIP1 nucleic acid with a KChIP1 binding agent; an agent that alters transcription of splicing variants encoded by a KChIP1 nucleic acid; and a ribozyme.

21. A pharmaceutical composition comprising a Type II diabetes therapeutic agent of claim 20.

22. The pharmaceutical composition of claim 21, wherein the Type II diabetes therapeutic agent is an isolated nucleic acid molecule comprising a KChIP1 nucleic acid or fragment or derivative thereof.

23. The pharmaceutical composition of claim 21, wherein the Type II diabetes therapeutic agent is a polypeptide encoded by the KChIP1 nucleic acid.

24. A method of treating a disease or condition associated with KChIP1 in an individual, comprising administering a Type II diabetes therapeutic agent to the individual, in a therapeutically effective amount.

25. The method of claim 24, wherein the Type II diabetes therapeutic agent is a KChIP1 nucleic acid agonist.

26. The method of claim 24 wherein the Type II diabetes therapeutic agent is a KChIP1 nucleic acid antagonist.

27. A transgenic animal comprising a nucleic acid selected from the group consisting of: an exogenous KChIP1 nucleic acid and a nucleic acid encoding a KChIP1 polypeptide.

28. A method for assaying a sample for the presence of a KChIP1 nucleic acid, comprising:
a) contacting said sample with a nucleic acid comprising a contiguous nucleotide sequence which is at least partially complementary to a part of the sequence of said KChIP1 gene under conditions appropriate for hybridization, and
b) assessing whether hybridization has occurred between a KChIP1 gene nucleic acid and said nucleic acid comprising a contiguous nucleotide sequence which is at least partially complementary to a part of the sequence of said KChIP1 nucleic acid;
wherein if hybridization has occurred, a KChIP1 nucleic acid is present in the nucleic acid.

29. The method of claim 28, wherein said nucleic acid comprising a contiguous nucleotide sequence is completely complementary to a part of the sequence of said KChIP1 nucleic acid.

30. The method of claim 28, further comprising amplification of at least part of said KChIP1 nucleic acid.

31. The method of claim 28, wherein said contiguous nucleotide sequence is 100 or fewer nucleotides in length and is either: a) at least 80% identical to a contiguous sequence of nucleotides in one of the nucleic acid sequences as shown in Table 10; b) at least 80% identical to the complement of a contiguous sequence of nucleotides in one of the nucleic acid sequences as shown in Table 10; or c) capable of selectively hybridizing to said KChIP1 nucleic acid.

32. A reagent for assaying a sample for the presence of a KChIP1 nucleic acid, said reagent comprising a nucleic acid comprising a contiguous nucleotide sequence which is at least partially complementary to a part of the nucleotide sequence of said KChIP1 nucleic acid.

33. The reagent of claim 32, wherein the nucleic acid comprises a contiguous nucleotide sequence, which is completely complementary to a part of the nucleotide sequence of said KChIP1 nucleic acid.

34. A reagent kit for assaying a sample for the presence of a KChIP1 nucleic acid, comprising in separate containers:
a) one or more labeled nucleic acids comprising a contiguous nucleotide sequence which is at least partially complementary to a part of the nucleotide sequence of said KChIP1 nucleic acid, and
b) reagents for detection of said label.

35. The reagent kit of claim 34, wherein the labeled nucleic acid comprises a contiguous nucleotide sequences which is completely complementary to a part of the nucleotide sequence of said KChIP1 nucleic acid.

36. A reagent kit for assaying a sample for the presence of a KChIP1 nucleic acid, comprising one or more nucleic acids comprising a contiguous nucleic acid sequence which is at least partially complementary to a part of the nucleic acid sequence of said KChIP1 nucleic acid, and which is capable of acting as a primer for said KChIP1 nucleic acid when maintained under conditions for primer extension.

37. The use of a nucleic acid which is 100 or fewer nucleotides in length and which is either: a) at least 80% identical to a contiguous sequence of nucleotides in one of the nucleic acid sequences as shown in Table 10; b) at least 80% identical to the complement of a contiguous sequence of nucleotides in one of the nucleic acid sequences as shown in Table 10; or c) capable of selectively hybridizing to said KChIP1 nucleic acid, for assaying a sample for the presence of a KChIP1 nucleic acid.

38. The use of a first nucleic acid which is 100 or fewer nucleotides in length and which is either:
a) at least 80% identical to a contiguous sequence of nucleotides in one of the nucleic acid sequences as shown in Table 6;
b) at least 80% identical to the complement of a contiguous sequence of nucleotides in one of the nucleic acid sequences as shown in Table 10; or
c) capable of selectively hybridizing to said KChIP1 nucleic acid; for assaying a sample for the presence of a KChIP1 nucleic acid that has at least one nucleotide difference from the first nucleic acid.

39. The use of a nucleic acid which is 100 or fewer nucleotides in length and which is either:
a) at least 80% identical to a contiguous sequence of nucleotides in one of the nucleic acid sequences as shown in Table 10;
b) at least 80% identical to the complement of a contiguous sequence of nucleotides in one of the nucleic acid sequences as shown in Table 10; or
c) capable of selectively hybridizing to said KChIP1 nucleic acid; for diagnosing a susceptibility to a disease or condition associated with a KChIP1.

40. A method of diagnosing a susceptibility to Type II diabetes in an individual, comprising determining the presence or absence in the individual of a haplotype comprising a halotype shown in Table 2, Table 4, Table 5, Table 14, Table 16, Table 17 and Table 18 at the 5q35 loci, wherein the presence of the haplotype is diagnostic of susceptibility to Type II diabetes.

41. The method of claim 40, wherein the haplotype comprises a least one of the polymorphisms indicated in Table 26.

42. The method of claim 40, wherein determining the presence or absence of the haplotype comprises enzymatic amplification of nucleic acid from the individual.

43. The method of claim 40, wherein determining the presence or absence of the haplotype further comprises electrophoretic analysis.

44. The method of claim 40, wherein determining the presence or absence of the haplotype further comprises restriction fragment length polymorphism analysis.

45. The method of claim 40, wherein determining the presence or absence of the haplotype further comprises sequence analysis.

46. A method of diagnosing a susceptibility to Type II diabetes in an individual, comprising:
a) obtaining a nucleic acid sample from said individual; and
b) analyzing the nucleic acid sample for the presence or absence of a haplotype, comprising a haplotype shown in Table 2, Table 4, Table 5, Table 14, Table 16, Table 17 or Table 18 at the 5q35 loci comprising a KChIP1 gene, wherein the presence of the haplotype is diagnostic for a susceptibility to Type II diabetes.

47. The method of claim 46, wherein the haplotype comprises the presence of a least one of the polymorphisms indicated in Table 26.

48. A method of diagnosing a susceptibility to Type II diabetes in an individual, comprising determining the presence or absence in the individual of a haplotype comprising one or more markers and/or single nucleotide polymorphisms as shown in Table 13 in the locus on chromosome 5q35, wherein the presence of the haplotype is diagnostic of a susceptibility to Type II diabetes.

49. The method of claim 48, wherein haplotype comprises the presence of a least one of the polymorphisms indicated in Table 26.

50. A method for the diagnosis and identification of a susceptibility to Type II diabetes in an individual, comprising: screening for an at-risk haplotype in the KChIP1 nucleic acid that is more frequently present in an individual susceptible to Type II diabetes compared to an individual who is not susceptible to Type II diabetes wherein the at-risk haplotype increases the risk significantly.

51. The method of claim 50, wherein the KChIP1 nucleic acid comprises at least one of the polymorphisms indicated in Table 26.

52. The method of claim 50 wherein the significant increase is at least about 20%.

53. The method of claim 50 wherein the significant increase is identified as an odds ratio of at least about 1.2.

54. Use of a Type II diabetes therapeutic agent for the manufacture of a medicament for the treatment of a disease or condition associated with KChIP1 in an individual.

55. The use of claim 54, wherein the Type II diabetes therapeutic agent is a KChIP1 nucleic acid agonist.

56. The use of claim 55, wherein the Tpe II diabetes therapeutic agent is a KChIP1 antagonist.

57. A method of diagnosing a predisposition or susceptibility to Type II diabetes in a subject, comprising detecting the presence or absence of a genetic marker associated with the KChIP1 gene, the marker having a p-value of 1×10⁻⁵ or less, wherein the presence of the marker associated with the KChIP1 gene is indicative of a predisposition or susceptibility to Type II diabetes.
